(19) United States
(12) Patent Application Publication
(10) Pub. No.: US 2023/0183239 A1
(43) Pub. Date: Jun. 15, 2023

(54) ECTONUCLEOTIDE PYROPHOSPHATASE-PHOSPHODIESTERASE 1 (ENPP-1) INHIBITORS AND USES THEREOF

(71) Applicant: AbbVie Inc., North Chicago, IL (US)

(72) Inventors: William Michael GALLATIN, Kirkland, WA (US); Joshua ODINGO, Kirkland, WA (US); Gregory N. DIETSCH, Kirkland, WA (US); Vincent FLORIO, Kirkland, WA (US); Chandregowda VENKATESHAPPA, Karnataka (IN); Athisayamani Jeyaraj DURAISWAMY, Karnataka (IN)

(21) Appl. No.: 17/702,597

(22) Filed: Mar. 23, 2022

Related U.S. Application Data
(63) Continuation of application No. 16/639,944, filed on Feb. 18, 2020.
(60) Provisional application No. 62/688,662, filed on Jun. 22, 2018.
(60) Provisional application No. 62/553,043, filed on Aug. 31, 2017.

(51) Int. Cl. C07D 471/10; C07D 211/28; C07D 215/233; C07D 215/44; C07D 239/88; C07D 239/94; C07D 241/42; C07D 307/81; C07D 311/22; C07D 401/04; C07D 401/14; C07D 405/14; C07D 409/12; C07D 413/14; C07D 471/04; C07D 487/04; C07D 519/00; C07F 5/02

(52) U.S. Cl. CPC C07D 471/10; C07D 211/28; C07D 215/44; C07D 215/233; C07D 239/88; C07D 239/94; C07D 241/42; C07D 307/81; C07D 311/22; C07D 401/04; C07D 401/14; C07D 405/14; C07D 409/12; C07D 413/14; C07D 471/04; C07D 487/04; C07D 519/00; C07F 5/025

(57) ABSTRACT

Disclosed herein are methods and compounds of augmenting and enhancing the production of type I IFNs in vivo. In some embodiments, the compounds disclosed herein are ENPP-1 inhibitors, pharmaceutical compositions, and methods for the treatment of cancer or a viral infection.

### CROSS REFERENCE

This application claims the benefit of U.S. Application Serial No. 62/553,043, filed Aug. 31, 2017 and U.S. Application Serial No. 62/688,662, filed Jun. 22, 2018, each of which are hereby incorporated by reference in their entirety.

### BACKGROUND OF THE DISCLOSURE

Cancer immunotherapy comprises the use of the patient’s immune system to combat tumor cells. In some instances, cancer immunotherapy utilizes the presence of tumor antigens (e.g., tumor-specific antigens) to facilitate the recognition of the tumor cells by the immune system. In other instances, cancer immunotherapy utilizes immune system components such as lymphocytes and cytokines to coordinate a general immune response.

Microorganisms that are capable of causing disease are termed pathogens. Pathogenic microorganisms include bacteria, virus, fungi, protozoa, and helminths. In some instances, antimicrobials such as broad spectrum fluoroquinolones and oxazolidinones fight infection by inhibiting microbial reproduction within a host. In other instances, antimicrobials enhance or strengthen a host’s immune response to the pathogenic infection.

### SUMMARY OF THE DISCLOSURE

Disclosed herein is a compound of Formula (X), or a pharmaceutically acceptable salt, solvate, or stereoisomer thereof:

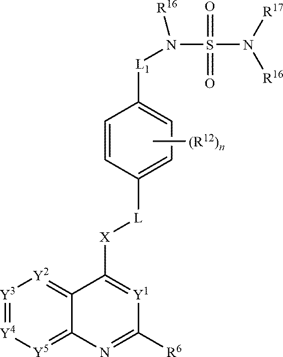

wherein
X is -NR⁷-, -O-, -S-, —S(═O)—, —S(═O)₂—, or -CR⁸R⁹-;
L is a bond or -CR¹⁰R¹¹-;
L₁ is a bond or -CR¹³R¹⁴-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, or optionally substituted heterocycloalkyl;
R¹⁰ and R¹¹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹² is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R⁷ and one R¹² are taken together to form an optionally substituted heterocycloalkyl or an optionally substituted heteroaryl; and the remaining R¹² are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl
R¹³ and R¹⁴ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹⁵ is hydrogen, deuterium, C₁-C₆ alkyl, C₁-C₆ haloalkyl, optionally substituted C₂-C₆ alkenyl, optionally substituted C₂-C₆ alkynyl, optionally substituted cycloalkyl, or optionally substituted heterocycloalkyl;
R¹⁶ and R¹⁷ are independently hydrogen, —C(═O)R^{a}, —C(═O)OR^{b}, —C(═O)NR^{c}R^{d}, C₁-C₆ alkyl, C₁-C₆ haloalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

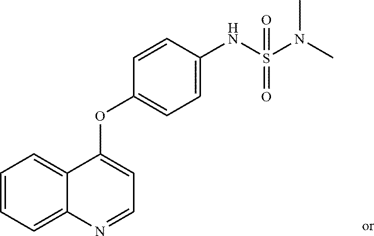

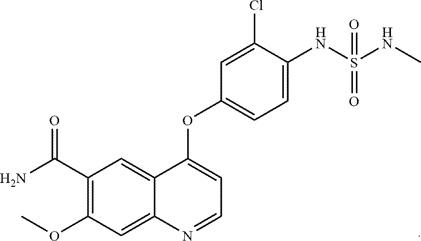

Also disclosed herein is a compound of Formula (VI), or a pharmaceutically acceptable salt, solvate, or stereoisomer thereof:

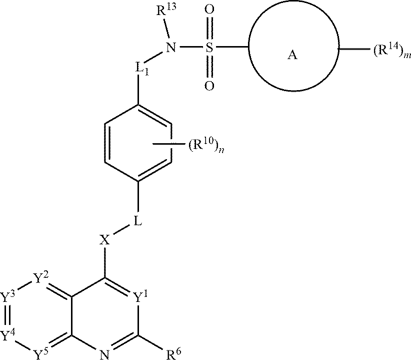

wherein
Ring A is cycloalkyl;
X is -NR⁷-, -O-, -S-, —S(═O)—, or —S(═O)₂—;
L is a bond or -CR⁸R⁹-;
L₁ is a bond or -CR¹¹R¹²-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁰ is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R⁷ and one R¹⁰ are taken together to form an optionally substituted heterocycloalkyl or an optionally substituted heteroaryl; and the remaining R¹⁰ are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹¹ and R¹² are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹³ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁴ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
m is 0-4;
each R^{a} is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

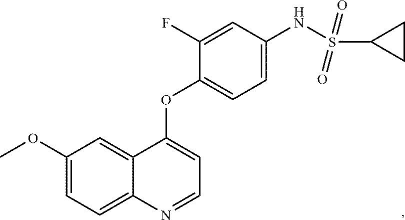

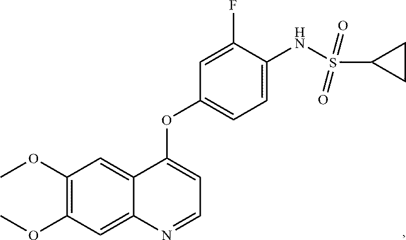

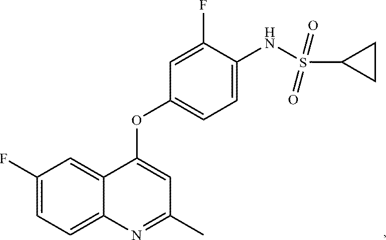

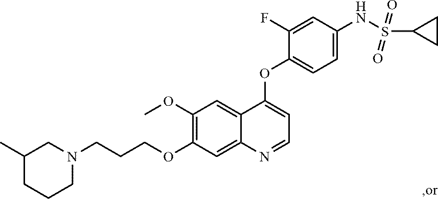

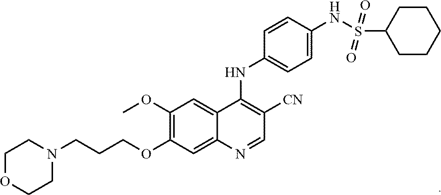

Also disclosed herein is a compound of Formula (VII), or a pharmaceutically acceptable salt, solvate, or stereoisomer thereof:

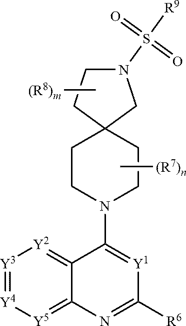

wherein
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R⁷ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-6;
each R⁸ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
m is 0-4;
R⁹ is OR¹⁰, NR¹¹R¹², optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹⁰ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹¹ and R¹² are independently hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

Also disclosed herein is a compound of Formula (VIII), or a pharmaceutically acceptable salt, solvate, or stereoisomer thereof:

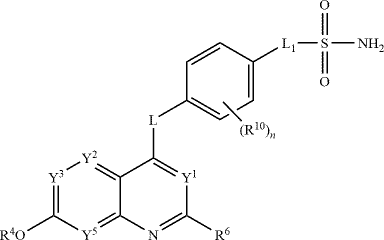

wherein
L is a bond, -O-, -S-, —S(═O)—, —S(═O)₂—, -O(CR⁸R⁹)-, -S(CR⁸R⁹)-, or -NR⁷(CR⁸R⁹)-;
L₁ is a bond, -O-, or -CR¹¹R¹²-;
Y¹ is -N- or -CR¹-;
Y² is -N- or -CR²-;
Y³ is -N- or -CR³-;
Y⁵ is -N- or -CR⁵-;
R¹, R², R³, and R⁵ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁴ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁶ is hydrogen, deuterium, halogen, -CN, -OR^{b}, -NO₂, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁰ is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹¹ and R¹² are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

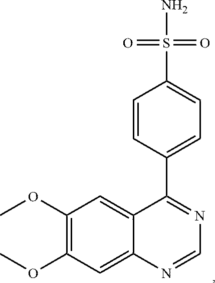

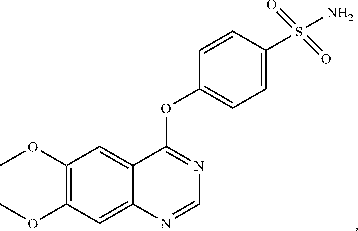

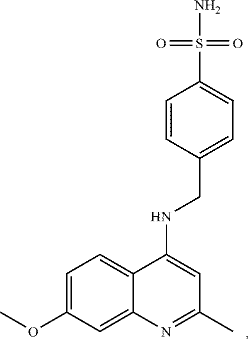

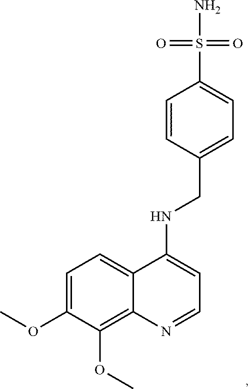

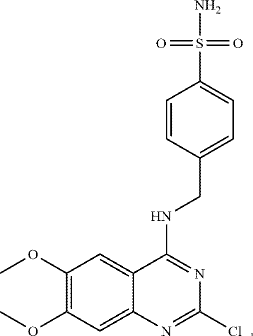

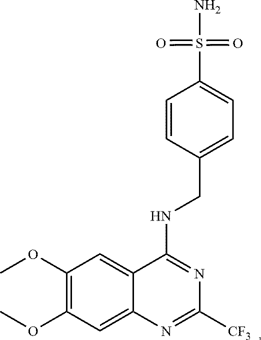

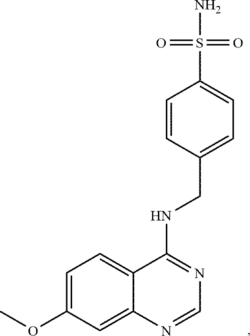

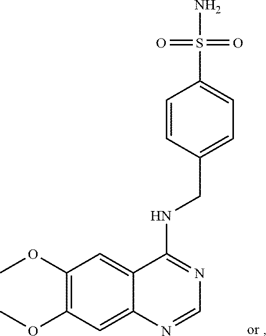

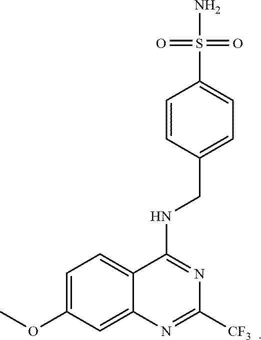

Also disclosed herein is a compound of Formula (IX), or a pharmaceutically acceptable salt, solvate, or stereoisomer thereof:

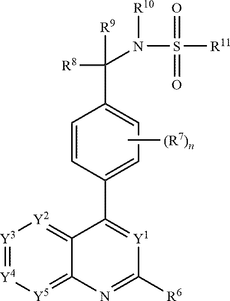

wherein
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹⁰ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl
or R¹⁰ and one R⁷ are taken together to form an optionally substituted heterocycloalkyl; and the remaining R¹⁰ are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹¹ is -OR¹², NR¹³R¹⁴, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹² is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹³ and R¹⁴ are independently hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹³ and R¹⁴ are taken together with the nitrogen atom to which they are attached to form an optionally substituted heterocycloalkyl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

Also disclosed herein is a compound of Formula (XI), or a pharmaceutically acceptable salt, solvate, or stereoisomer thereof:

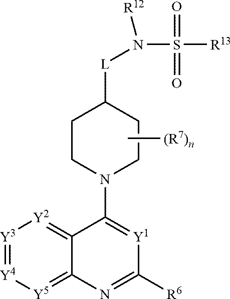

wherein
L is -(CR⁸R⁹)(CR¹⁰R¹¹)-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R⁷ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-6;
R⁸, R⁹, R¹⁰, and R¹¹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹² is hydrogen, optionally substituted C₁-C₆ alkyl, optionally substituted C₂-C₆ alkenyl, optionally substituted C₂-C₆ alkynyl, optionally substituted cycloalkyl, or optionally substituted heterocycloalkyl;
R¹³ is -OR¹⁴, NR¹⁵R¹⁶, or optionally substituted cycloalkyl;
R¹⁴ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl,- optionally substituted heterocycloalkyl, optionally substituted aryl, or optionally substituted heteroaryl;
R¹⁵ is optionally substituted cycloalkyl;
R¹⁶ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

Also disclosed herein is a pharmaceutical composition comprising a compound disclosed herein and a pharmaceutically acceptable excipient.

Also disclosed herein is a method of treating cancer in a subject in need thereof comprising administering a compound disclosed herein or a pharmaceutical composition comprising a compound disclosed herein.

Also disclosed herein is a method of treating an infection in a subject in need thereof comprising administering a compound disclosed herein or a pharmaceutical composition comprising a compound disclosed herein.

### BRIEF DESCRIPTION OF THE DRAWINGS

Included in the drawings are the following figures.

FIG. 1 depicts that the ENPP-1 blockade by Example 54 enhances transcription of IFNβ at 3, 6, and 19 hrs exposure in VACV-70 stimulated PBMCs.

FIG. 2 depicts that the ENPP-1 blockade by Example 58 enhances transcription of IFNβ at 3, 6, and 19 hrs exposure in VACV-70-stimulated PBMCs.

FIG. 3 depicts that the ENPP1 blockade by Example 57 enhances .transcription of IFNβ in cGAMP-stimulated human foreskin fibroblast cells (HFF-1)

FIG. 4 depicts that the ENPP1 blockade by Example 55 enhances transcription of IFNβ in cGAMP-stimulated human foreskin fibroblast cells (HFF-1).

FIG. 5 depicts that the ENPP 1 blockade by Example 32 enhances transcription of IFNβ in cGAMP-stimulated human foreskin fibroblast cells (HFF-1).

### DETAILED DESCRIPTION OF THE DISCLOSURE

In some embodiments, the immunophenotype of a tumor microenvironment modulates the responsiveness of the tumor to a cancer therapy. In some instances, tumor-infiltrating lymphocytes are correlated with favorable prognosis in different types of tumors and are correlated with positive clinical outcome in response to several lines of immunotherapy.

In some cases, innate immune sensing in the tumor microenvironment promotes T-cell priming and subsequent infiltration of tumor-infiltrating lymphocytes. For example, transcriptional profiling analyses of melanoma patients have shown that tumors containing infiltrating activated T cells are characterize by a type I IFN transcriptional signature. Furthermore, mice lacking the IFN-α/β receptor in dendritic cells are unable to reject immunogenic tumors and the CD8α+ dendritic cells from these mice are defective in antigen cross-presentation to CD8+ T cells.

In some embodiments, systemic delivery of type I IFNs has shown efficacy in cancer settings. Indeed, systemic injection of IFN-β in a mouse xenograft model of human colorectal cancer liver metastases has shown tumor regression and improved survival.

In some instances, systemic delivery of type I IFNs requires high doses to achieve therapeutic benefit. In such cases, desensitization of the immune system and issues with tolerability have also been observed.

The innate immune system is the first line of defense to a microbial infection. The host innate immunity is activated through recognition of conserved microbial signatures termed pathogen-associated molecular patterns (PAMPs) and host damage-associated molecular patterns (DAMPs). Upon sensing of microbial PAMPs and DAMPs, signal cascades are activated to produce type I interferons and/or multiple cytokines and chemokines, culminating in the synthesis of antiviral proteins. The presence of antiviral proteins and cytokines (e.g., interferons or chemokines) subsequently promote apoptosis, inhibits cellular protein translation, and recruit immune cells to the site of infection to further initiate adaptive immune response.

Pattern recognition receptors (PRRs) are germ-line encoded receptors that recognize PAMPs and DAMPs and facilitate the rapid and efficient innate immune response. Cytosolic DNA sensor is a type of PRRs that detects the intracellular presence of pathogenic DNA. DNA-dependent activator of IFN-regulatory factors (DAI), a cytosolic DNA sensor, utilizes the cGAS-STING pathway for production of type I interferons.

In some embodiments, disclosed herein are methods of enhancing and/or augmenting the production of type I IFNs in vivo, without the need of systemic delivery of type I IFNs. In such instances, the IFN production is localized in the tumor microenvironment. In some cases, the methods comprise activating and enhancing the cGAS-STING response. In some cases, the methods comprise priming a cancer with an immunogenic cell death inducer prior to stimulating the cGAS-STING pathway. In other cases, the methods comprise blocking the degradation of a STING activating substrate prior to priming a cancer with an immunogenic cell death inducer. In additional cases, the methods comprise use of an inhibitor of a 2′3′-cGAMP degradation polypeptide (e.g., an inhibitor of a phosphodiesterase) with an immunogenic cell death inducer for the treatment of a cancer.

In additional embodiments, disclosed herein include methods of designing inhibitors of 2′3′-cGAMP degradation polypeptides and assays for evaluating the enzyme activity of the GMP degradation polypeptides.

### cGAS-STING Pathway, Immunogenic Cell Death, and the Production of Type I IFNs

Cytosolic DNA can signal the presence of cellular damage and/or the presence of cancerous cells and/or an infection within a cell or at a nearby cell. These cytosolic DNAs (e.g., double stranded DNAs) are surveyed by DNA sensors such as RNA pol III, DAI, IFI16, DDX41, LSm14A, cyclic-GMP-AMP synthase, LRRFIP1, Sox2, DHX9/36, Ku70 and AIM2. Cyclic-GMP-AMP synthase (cGAS or cGAMP synthase) is a 522 amino acid protein that belongs to the nucleotidyltransferase family of cytosolic DNA sensors. Upon cytosolic DNA stimulation, cGAS synthesizes cGAMP, which comprises a first bond between the 2′-OH of GMP and the 5′-phosphate of AMP and a second bond between the 3′-OH of AMP and the 5′-phosphate of GMP. cGAMP (also known as cyclic GMP-AMP, 2′3′-cGAMP, cGAMP (2′-5′) or cyclic Gp(2′-5′)Ap(3′-5′)) serves as a ligand to STING, thereby activating the STING-mediated IFN (e.g., IFNβ) production.

STING (also known as stimulator of interferon genes, TMEM173, MITA, ERIS, or MPYS) is a 378 amino acid protein that comprises a N-terminal region containing four trans-membrane domains and a C-terminal domain that comprises a dimerization domain. Upon binding to 2′3′-cGAMP, STING undergoes a conformational rearrangement enclosing the 2′3′-cGAMP molecule.

Binding of 2′3′-cGAMP activates a cascade of events whereby STING recruits and activates IκB kinase (IKK) and TANK-binding kinase (TBK1), which following their phosphorylation, respectively activate nuclear transcription factor κB (NF-κB) and interferon regulatory factor 3 (IRF3). In some instances, the activated proteins translocate to the nucleus to induce transcription of the genes encoding type I IFN and cytokines for promoting intercellular host immune defense. In some cases, the production of type I IFNs further drives the development of cytolytic T cell response and enhances expression of MHC, thereby increasing antigen processing and presentation within a tumor microenvironment. In such cases, enhanced type I IFN production further renders the tumor cells to be more vulnerable by enhancing their recognition by the immune system.

In some instances, STING is capable of directly sensing bacterial cyclic dinucleotides (CDNs) such as c[di-GMP]. In some cases, 2′3′-cGAMP acts as a second messenger binding to STING in response to cells exposed to cytosolic DNA.

In some embodiments, cytosolic DNA is generated through “self-DNA” or endogenous DNA from the host through the DNA structure-specific endonuclease methyl methane-sulphonate (MMS) and ultraviolet-sensitive 81 (MUS81). The DNA structure-specific endonuclease MUS81 is a member of the XPF family of endonucleases that forms a heterodimeric complex with essential meiotic endonuclease 1 (EME1). In some instances, the MUS81-EME1 complex cleaves DNA structures at stalled replication forks. In some cases, MUS81 cleavage of self-DNA leads to accumulation of cytosolic DNA and activation of the STING pathway.

In other instances, cytosolic DNA is generated through immunogenic cell death (ICD)-mediated events, activation of the STING-pathway, production of type I INFs, and further priming of the tumor cell microenvironment.

### Immunogenic Cell Death

In some embodiments, immunogenic cell death (ICD), or immunogenic cancer cell death, is a cell death modality which further stimulates an immune response against tumor expressed antigens. In some cases, tumor expressed antigens are tumor neoantigens or antigens that are formed by mutated proteins and unique to the tumor. In other cases, tumor expressed antigens comprise overexpressed proteins such as MUC1, CA-125, MART-1 or carcinoembyonic antigen (CEA). In some instances, ICD is characterized by a series of biochemical events that comprises: 1) the cell surface translocation of calreticulin (CALR or CRT), an endoplasmic reticulum (ER) resident chaperone protein and a potent DC “eat me” signal; 2) the extracellular release of high mobility group box 1 (HMGB1), a DNA binding protein and toll-like receptor 4 (TLR-4) mediated DC activator; and 3) the liberation of adenosine-5 ′-triphosphate (ATP), a cell-cell signaling factor in the extracellular matrix (ECM) that serves to activate P2X7 purinergic receptors on DCs, triggering DC inflammasome activation, secretion of IL-Iβ, and subsequent priming of interferon-γ (IFNy) producing CD8⁺ T cells. In some embodiments, the cumulative effects of the 3 arms of ICD and in particular CRT exposure (or the surface translocation of CRT) act to promote DC phagocytosis of tumor cells, thereby facilitating DC processing of tumor-expressed antigens and subsequent DC-associated cross-priming of CD8⁺ cytotoxic T lymphocytes.

Calreticulin, also known as calregulin, CRP55, CaBP3, calsequestrin-like protein, and endoplasmic reticulum resident protein 60 (ERp60), is a protein that in humans is encoded by the CALR gene. Calreticulin is a multifunctional protein that binds Ca²⁺ ions (a second messenger in signal transduction), rendering it inactive. In some instances, calreticulin is located in the lumen of the endoplasmic reticulum, where it interacts with misfolded proteins, inhibits their export from the endoplasmic reticulum into the Golgi apparatus and subsequently tags these misfolded proteins for degradation. In some cases, calreticulin further serves as a signaling ligand to recruit DCs to initiate phagocytosis.

In some embodiments, ICD is further sub-categorized into different types of ICD based on the ICD inducer. In some instances, an ICD inducer initiates the process of immunogenic cell death. In some cases, an ICD inducer comprises radiation. Exemplary types of radiation include UV radiation and γ radiation. In some cases, an ICD inducer comprises UV radiation. In some cases, an ICD inducer comprises γ radiation.

In other cases, an ICD inducer comprises a small molecule. In some cases, the small molecule comprises a chemotherapeutic agent. Exemplary chemotherapeutic agents include, but are not limited to, an anthracycline such as doxorubicin or mitoxantrone; a cyclophosphamide such as mafosfamide; bortezomib, daunorubicin, docetaxel, oxaliplatin or paclitaxel. In some instances, an ICD inducer comprises doxorubicin, mitoxantrone, mafosfamide, bortezomib, daunorubicin, docetaxel, oxaliplatin, paclitaxel, or any combinations thereof. In some instances, an ICD inducer comprises digitoxin or digoxin. In some instances, an ICD inducer comprises digitoxin. In some instances, an ICD inducer comprises digoxin. In some instances, an ICD inducer comprises septacidin. In some cases, an ICD inducer comprises a combination of cisplatin and thapsigargin. In some cases, an ICD inducer comprises a combination of cisplatin and tunicamycin.

In additional cases, an ICD inducer comprises a biologic. In such cases, a biologic comprises a protein or functional fragments thereof, a polypeptide, an oligosaccharide, a lipid, a nucleic acid (e.g., DNA or RNA) or a protein-payload conjugate. In some cases, a protein or functional fragments thereof comprises an enzyme, a glycoprotein, or a protein capable of inducing ICD. In some cases, a protein or functional fragments thereof comprises a humanized antibody or binding fragment thereof, a chimeric antibody or binding fragment thereof, a veneer antibody or binding fragment thereof, a monoclonal antibody or binding fragment thereof, a bispecific antibody or binding fragment thereof, an Fab, an Fab′, an F(ab′)₂, an F(ab′)₃, an scFv, an sc(Fv)₂, a dsFv, a diabody, a minibody, or a nanobody or binding fragments thereof. In some cases, a protein-payload conjugate comprises a protein or functional fragments thereof conjugated to a payload (e.g., a small molecule payload). In some cases, an exemplary protein-payload conjugate is trastuzumab emtansine.

In some embodiments, CRT exposure leads to phagocytosis by dendritic cells, leading to generating a population of cytosolic DNA. In some cases, cytosolic DNA sensor such as cyclic GMP-AMP synthase detects the presence of the cytosolic DNA and subsequently triggers inflammatory responses (e.g., generation of type I IFNs) via the STING-mediated pathway.

### Pathogens

As described above, the presence of intracellular nucleic acid from a pathogen activates cGAS, leading to production of 2′3′-cGAMP, and subsequent activation of the STING pathway. In some instances, the pathogen is a virus, e.g., a DNA virus or an RNA virus. In some cases, the pathogen is a retrovirus. Exemplary viruses capable of subsequent activation of STING include, but are not limited to, herpes simplex virus 1 (HSV-1), murine gamma-herpesvirus 68 (MHV68), Kaposi’s sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In other instances, the pathogen is a bacterium. Exemplary bacteria include, but are not limited to, Listeria monocytogenes, Mycobacterium tuberculosis, Francisella novicida, Legionella pneumophila, Chlamydia trachomatis, Streptococcus pneumoniae, or Neisseria gonorrhoeae.

In some embodiments, the pathogen is a DNA virus. In some instances, the DNA virus is a single-stranded DNA virus. In other instances, the DNA virus is a double-stranded DNA virus. In some cases, the virus utilizes a DNA-dependent DNA polymerase for replication.

In some embodiments, the pathogen is an RNA virus. In some instances, the RNA virus is a single-stranded RNA virus (e.g., single-stranded-positive sense or single-stranded-negative sense). In other instances, the RNA virus is a double-stranded RNA virus. Exemplary RNA viruses include vesicular stomatitis virus (VSV), sendai virus, hepatitis C virus, dengue fever virus, yellow fever virus, ebola virus, Marburg virus, venezuelan encephalitis virus, or zika virus. In some embodiments, the RNA virus is dengue fever virus, yellow fever virus, ebola virus, Marburg virus, Venezuelan encephalitis virus, or zika virus.

In some embodiments, the pathogen is a retrovirus. Retroviruses are single-stranded RNA viruses with a DNA intermediate. In most viruses, DNA is transcribed into RNA, and then RNA is translated into protein. However, retroviruses function differently, as their RNA is reverse-transcribed into DNA. Upon infection of a cell by a retrovirus, the retroviral RNA genome is transcribed into its corresponding double-stranded DNA by a reverse transcriptase enzyme which is coded for by the viral genome, which is the reverse of the usual pattern, thus retro (backwards). This DNA then enters the nucleus and integrates into the host DNA using an integrase enzyme which is also coded for by the viral genome. The integrated viral DNA (“proviral” DNA) becomes a component of the host genome, replicating with it and producing the proteins required in assembling new copies of the virus. It is difficult to detect the virus until it has infected the host. The information contained in a retroviral gene is thus used to generate the corresponding protein via the sequence: RNA → DNA → RNA → polypeptide.

The genome of a retrovirus (in either the RNA or DNA form) is divided conceptually into two parts. The first, or “trans-acting,” category consists of the regions coding for viral proteins. These include the group specific antigen (“gag”) gene for synthesis of the core coat proteins, the “pol” gene for the synthesis of various enzymes (such as reverse transcriptase), and the envelope (“env”) gene for the synthesis of envelope glycoproteins. The full-length RNA transcript is packaged by the viral proteins into a viral particle which then buds off in a piece of cell membrane, in which are embedded env-derived peptides. This membrane-coated viral particle is a fully competent viral particle and goes on to infect other cells.

In general, the second part of the retroviral genome is referred to as the “cis-acting” portion and consists of the regions which must be on the genome to allow its packaging and replication. This includes the packaging signal on an RNA molecule, such as the viral RNA, which identifies that RNA molecule to viral proteins as one to be encapsidated, Long Terminal Repeats (“LTRs”) with promoters and polyadenylation sites, and two start sites for DNA replication. The promoters, enhancers, and other regions of the LTRs are also capable of conferring tissue specificity, such that the virus will only be “expressed” (i.e., transcribed and translated) in specific cell types even though it infects others.

### Exemplary Viruses

### Herpes Simplex Virus 1 (HSV-1)

HSV-1 is a highly contagious infection, which is common and endemic throughout the world. Most HSV-1 infections are acquired during childhood. The vast majority of HSV-1 infections are oral herpes (infections in or around the mouth, sometimes called orolabial, oral-labial or oral-facial herpes), but a proportion of HSV-1 infections are genital herpes (infections in the genital or anal area). HSV-1 is mainly transmitted by oral-to-oral contact to cause oral herpes infection, via contact with the HSV-1 virus in sores, saliva, and surfaces in or around the mouth. However, HSV-1 is also transmitted to the genital area through oral-genital contact to cause genital herpes.

### Murine Gamma-Herpesvirus 68 (MHV68)

MHV-68 is a rodent pathogen and a member of the gammaherpesvirus subfamily. MHV-68 has the ability to establish latent infections within lymphocytes and make close associations with cell tumors. MHV-68 establishes latency unless the host immune system is compromised, and this latency is regulated by multiple cellular controls, such as virus-specific open reading frames that result in gene products promoting the maintenance of latency or activation of lytic cycles. One of the major consequences of MHV-68 in mice is infectious mononucleosis. MHV-68 infection sites consist of primarily lung epithelial cells, adrenal glands, and heart tissue, with latent infection in B lymphocytes.

### Kaposi’s Sarcoma-Associated Herpesvirus (KSHV)

KSHV or human herpesvirus 8 (HHV8) is a human rhadinovirus (gamma-2 herpesvirus) belonging to the family of herpesviruses. KSHV is a large double-stranded DNA virus with a protein covering that packages its nucleic acids, called the capsid, which is then surrounded by an amorphous protein layer called the tegument, and finally enclosed in a lipid envelope derived in part from the cell membrane. This virus is transmitted both sexually and through body fluids, (for example, saliva and blood). KSHV causes a blood vessel cancer called Kaposi’s sarcoma (KS), a lymphoma (a cancer of the lymphocyte) called body cavity-based lymphoma, and some forms of severe lymph node enlargement, called Castleman’s disease.

### Vaccinia Virus (VACV)

Vaccinia virus (VACV or VV) is a large, complex, enveloped virus belonging to the poxvirus family. The poxviruses are the largest known DNA viruses and are distinguished from other viruses by their ability to replicate entirely in the cytoplasm of infected cells. Poxviruses do not require nuclear factors for replication and, thus, replicate with little hindrance in enucleated cells. VACV has a linear, double-stranded DNA genome of approximately 190 kb in length, which encodes for around 250 genes. The genome is surrounded by a lipoprotein core membrane. Vaccinia virus is well-known for its role as a vaccine that eradicated the smallpox disease. The natural host of Vaccinia virus is unknown, but the virus replicates in cows and humans. During its replication cycle, Vaccinia virus produces four infectious forms which differ in their outer membranes: intracellular mature virion (IMV), the intracellular enveloped virion (IEV), the cell-associated enveloped virion (CEV) and the extracellular enveloped virion (EEV).

### Adenovirus

Adenoviruses are double-stranded DNA viruses and are now known to be a common cause of asymptomatic respiratory tract infection. An extremely hardy virus, adenovirus is ubiquitous in human and animal populations, survives long periods outside a host, and is endemic throughout the year. Possessing 52 serotypes, adenovirus is recognized as the etiologic agent of various diverse syndromes. It is transmitted via direct inoculation to the conjunctiva, a fecal-oral route, aerosolized droplets, or exposure to infected tissue or blood. The virus is capable of infecting multiple organ systems; however, most infections are asymptomatic.

### Human Papillomaviruses (HPV)

Human papillomaviruses (HPV), DNA viruses from the papillomavirus family, are common viruses that cause warts. There are more than 100 types of HPV. Most are harmless, but about 30 types put one at risk for cancer. These types affect the genitals and one gets them through sexual contact with an infected partner. They are either low-risk or high-risk. Low-risk HPV causes genital warts. High-risk HPV leads to cancers of the cervix, vulva, vagina, and anus in women and cancers of the anus and penis in men.

### Hepatitis B Virus (HBV)

HBV, a member of the Hepadnaviridae family, is a small DNA virus with unusual features similar to retroviruses. HBV replicates through an RNA intermediate and integrates into the host genome. Hepatitis B is one of a few known non-retroviral viruses which use reverse transcription as a part of its replication process. The features of the HBV replication cycle confer a distinct ability of the virus to persist in infected cells. HBV infection leads to a wide spectrum of liver diseases ranging from acute (including fulminant hepatic failure) to chronic hepatitis, cirrhosis, and hepatocellular carcinoma. Acute HBV infection is either asymptomatic or presents with symptomatic acute hepatitis. About 5%-10% of population infected is unable to clear the virus and becomes chronically infected. Many chronically infected persons have mild liver disease. Other individuals with chronic HBV infection develop active disease, which progresses to cirrhosis and liver cancer.

### Hepatitis D Virus (HDV)

Hepatitis D virus (HDV) is a small spherical enveloped viroid. HDV is considered to be a subviral satellite because it can propagate only in the presence of the hepatitis B virus (HBV). Transmission of HDV can occur either via simultaneous infection with HBV (coinfection) or superimposed on chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B virus, hepatitis D has the highest fatality rate of all the hepatitis infections, at 20%.

### Human Immunodeficiency Virus (HIV)

The human immunodeficiency virus (HIV) is a lentivirus (a subgroup of retroviruses) that causes HIV infection and over time acquired immunodeficiency syndrome (AIDS). AIDS is a condition in humans in which progressive failure of the immune system allows life-threatening opportunistic infections and cancers to thrive. Infection with HIV occurs by the transfer of blood, semen, vaginal fluid, pre-ejaculate, or breast milk. Within these bodily fluids, HIV is present as both free virus particles and virus within infected immune cells. HIV infects vital cells in the human immune system such as helper T cells (specifically CD4⁺ T cells), macrophages, and dendritic cells. HIV infection leads to low levels of CD4⁺ T cells through a number of mechanisms, including but not limited to, pyroptosis of abortively infected T cells, apoptosis of uninfected bystander cells, direct viral killing of infected cells, and killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections.

### Human Cytomegalovirus (HCMV)

Human cytomegalovirus (HCMV) is a beta-herpesvirus that causes lifelong infection in humans. HCMV has a prevalence of 55-100% within the human population. Primary HCMV infection is generally asymptomatic in healthy hosts but causes severe and sometimes fatal disease in immunocompromised individuals, organ transplant recipients, and neonates. HCMV is the leading infectious cause of congenital abnormalities in the Western world, affecting 1-2.5% of all live births. After infection, HCMV remains latent within the body throughout life and is reactivated at any time. Eventually, it causes mucoepidermoid carcinoma and other malignancies such as prostate cancer. Although they are found throughout the body, HCMV infections are frequently associated with the salivary glands. The mode of HCMV transmission from person to person is entirely unknown but is presumed to occur through bodily fluids. Infection requires close, intimate contact with a person secreting the virus in their saliva, urine, or other bodily fluids. HCMV is transmitted sexually and via breast milk, and also occurs through receiving transplanted organs or blood transfusions.

### Dengue Fever Virus

Dengue fever virus (DENV) is an RNA virus of the family Flaviviridae; genus Flavivirus. It is transmitted by arthropods (mosquitoes or ticks), and is therefore also referred to as a arbovirus (arthropod-borne virus). Dengue virus is primarily transmitted by Aedes mosquitoes, particularly A. aegypti. Other Aedes species that transmit the disease include A. albopictus, A. polynesiensis, and A. scutellaris. Humans are the primary host of the virus but it also circulates in nonhuman primates. A female mosquito that takes a blood meal from a person infected with dengue fever, during the initial 2- to 10-day febrile period, becomes itself infected with the virus in the cells lining its gut. About 8-10 days later, the virus spreads to other tissues including the mosquito’s salivary glands and is subsequently released into its saliva. The virus seems to have no detrimental effect on the mosquito, which remains infected for life.

### Ebola Virus

Ebola virus (EBOV) is one of five known viruses within the genus Ebolavirus. Four of the five known ebolaviruses, including EBOV, cause a severe and often fatal hemorrhagic fever in humans and other mammals, known as Ebola virus disease (EVD). The EBOV genome is a single-stranded RNA approximately 19,000 nucleotides long. It encodes seven structural proteins: nucleoprotein (NP), polymerase cofactor (VP35), (VP40), GP, transcription activator (VP30), VP24, and RNA-dependent RNA polymerase (L).

### Marburg Virus

Marburg virus is a hemorrhagic fever virus of the Filoviridae family of viruses and a member of the species Marburg marburgvirus, genus Marburgvirus. Marburg virus (MARV) causes Marburg virus disease in humans and nonhuman primates, a form of viral hemorrhagic fever. The virus is considered to be extremely dangerous.

### Zika Virus

Zika virus (ZIKV) is a member of the virus family Flaviviridae. It is spread by daytime-active Aedes mosquitoes, such as A. aegypti and A. albopictus. Zika virus is related to the dengue, yellow fever, Japanese encephalitis, and West Nile viruses. Since the 1950s, it has been known to occur within a narrow equatorial belt from Africa to Asia. The infection, known as Zika fever or Zika virus disease, often causes no or only mild symptoms, similar to a very mild form of dengue fever. While there is no specific treatment, paracetamol (acetaminophen) and rest may help with the symptoms. Zika can spread from a pregnant woman to her baby. This can result in microcephaly, severe brain malformations, and other birth defects. Zika infections in adults may result rarely in Guillain-Barre syndrome.

### Bacteria

In some embodiments, a pathogen described herein is a bacterium. Bacteria are microscopic single-celled microorganisms that exist either as independent (free-living) organisms or as parasites (dependent on another organism for life) and thrive in diverse environments. As prokaryotes, the organism consists of a single cell with a simple internal structure. Bacterial DNA floats free, in a twisted thread-like mass called the nucleoid. Bacterial cells also contain separate, circular pieces of DNA called plasmids. Bacteria lack membrane-bound organelles, specialized cellular structures that are designed to execute a range of cellular functions from energy production to the transport of proteins. However, both bacterial cells contain ribosomes. A few different criteria are used to classify bacteria. They are distinguished by the nature of their cell walls, by their shape, or by differences in their genetic makeup.

### Exemplary Bacteria

### Listeria Monocytogenes

Listeria monocytogenes is the species of pathogenic bacteria that causes the infection listeriosis. L monocytogenes is a motile, non-spore-forming, gram-positive bacillus that has aerobic and facultative anaerobic characteristics making it capable of surviving in the presence or absence of oxygen. It grows best at neutral to slightly alkaline pH and is capable of growth at a wide range of temperatures, from 1-45° C. It is beta-hemolytic and has a blue-green sheen on blood-free agar. It exhibits characteristic tumbling motility when viewed with light microscopy. It grows and reproduces inside the host’s cells and is one of the most virulent foodborne pathogens, with 20 to 30% of food borne listeriosis infections in high-risk individuals are fatal. Most infections occur after oral ingestion, with access to the systemic circulation after intestinal penetration. CNS infection manifests as meningitis, meningoencephalitis, or abscess. Endocarditis is another possible presentation. Localized infection manifests as septic arthritis, osteomyelitis, and, rarely, pneumonia.

### Mycobacterium Tuberculosis

Mycobacterium tuberculosis is an obligate pathogenic bacterial species in the family Mycobacteriaceae and the causative agent of tuberculosis. M. tuberculosis has an unusual, waxy coating on its cell surface (primarily due to the presence of mycolic acid), which makes the cells impervious to Gram staining. The physiology of M. tuberculosis is highly aerobic and requires high levels of oxygen. Primarily a pathogen of the mammalian respiratory system, it infects the lungs.

### Francisella Novicida

Francisella novicida is a bacterium of the Francisellaceae family, which consist of Gram-negative pathogenic bacteria. These bacteria vary from small cocci to rod-shaped, and are most known for their intracellular parasitic capabilities. Some of the main symptoms associated with this infection include pneumonia, muscle pain, and fever.

### Legionella Pneumophila

Legionella pneumophila is a thin, aerobic, pleomorphic, flagellated, nonspore-forming, Gram-negative bacterium of the genus Legionella. L. pneumophila infection causes Legionnaires’ disease, a severe form of pneumonia. The symptoms of Legionnaire’s disease include confusion, headache, diarrhea, abdominal pain, fever, chills, and myalgia as well as a non-productive cough. Pontiac fever is a non-pneumonic form of L. pneumophila infection. Symptoms are flu-like, including fever, tiredness, myalgia, headache, sore throat, nausea, and sometimes cough. L. pneumophila is transmitted by aerosols and aspiration of contaminated water.

### Chlamydia Trachomatis

Chlamydia trachomatis is a gram-negative bacterium that infects the columnar epithelium of the cervix, urethra, and rectum, as well as nongenital sites such as the lungs and eyes. The bacterium is the cause of the most frequently reported sexually transmitted disease in the United States. Most persons with this infection are asymptomatic. Untreated infection results in serious complications such as pelvic inflammatory disease, infertility, and ectopic pregnancy in women, and epididymitis and orchitis in men. Men and women experience chlamydia-induced reactive arthritis. In neonates and infants, the bacterium causes conjunctivitis and pneumonia. Adults also experience conjunctivitis caused by chlamydia. Trachoma is a recurrent ocular infection caused by chlamydia.

### Streptococcus Pneumoniae

Streptococcus pneumoniae, or pneumococcus is a Gram-positive, alpha-hemolytic (under aerobic conditions) or beta-hemolytic (under anaerobic conditions), facultative anaerobic member of the genus Streptococcus, that is responsible for the majority of community-acquired pneumonia. It is a commensal organism in the human respiratory tract, meaning that it benefits from the human body, without harming it. However, infection by pneumococcus is dangerous, causing not only pneumonia, but also bronchitis, otitis media, septicemia, and meningitis. Pneumococcal pneumonia causes fever and chills, coughs, difficulty breathing, and chest pain. If the infection spreads to the brain and spinal cord, it causes pneumococcal meningitis, characterized by a stiff neck, fever, confusion, and headaches. S. pneumoniae primarily spreads through the air in the form of aerosol droplets from coughing and sneezing.

### Neisseria Gonorrhoeae

Neisseria gonorrhoeae, also known as gonococci (plural), or gonococcus (singular), is a species of Gram-negative, fastidious, coffee bean-shaped diplococci bacteria responsible for the sexually transmitted infection gonorrhea. Neisseria gonorrhoeae grow and rapidly multiply in the mucous membranes, especially the mouth, throat, and anus of males and females, and the cervix, fallopian tubes, and uterus of the female reproductive tract. N. gonorrhoeae is transmitted from person to person via oral, vaginal, and anal sexual contact. During childbirth, infants contract the infection in the birth canal resulting in bilateral conjunctivitis.

### Phosphodiesterases

In some embodiments, tumor cells circumvent the STING-mediated type I IFN production through overexpression of a phosphodiesterase. In some instances, phosphodiesterase has been linked with viral infection and its inhibition has been correlated with a reduction in viral replication. Phosphodiesterases comprise a class of enzymes that catalyze the hydrolysis of a phosphodiester bond. In some instances, this class comprises cyclic nucleotide phosphodiesterases, phospholipases C and D, autotaxin, sphingomyelin phosphodiesterase, DNases, RNases, restriction endonucleases, and small-molecule phosphodiesterases.

Cyclic nucleotide phosphodiesterases (PDEs) regulate the cyclic nucleotides cAMP and cGMP. In some instances, cAMP and cGMP function as intracellular second messengers to transduce a variety of extracellular signals including hormones, light, and neurotransmitters. In some cases, PDEs degrade cyclic nucleotides to their corresponding monophosphates, thereby regulating the intracellular concentrations of cyclic nucleotides and their effects on signal transduction.

In some embodiments, PDEs are classified into classes I, II and III. In some cases, mammalian PDEs, which belong to Class I PDEs, are further divided into 12 families (PDE1-PDE12) based on their substrate specificity and affinity, sensitivity to cofactors, and sensitivity to inhibitory agents. In some cases, the different families of mammalian PDEs further contain splice variants that can be unique in tissue-expression patterns, gene regulation, enzymatic regulation by phosphorylation and regulatory proteins, subcellular localization, and interaction with association proteins.

The PDE1 family comprises Ca²⁺/calmodulin-dependent PDEs. In some cases, PDE1 is encoded by at least three different genes, each having at least two different splice variants, PDE1A and PDE1B. In some cases, PDE1 isozymes are regulated in vitro by phosphorylation/dephosphorylation. For example, phosphorylation decreases the affinity of PDE for calmodulin, decreases the activity of PDE1, and increases steady state levels of cAMP. In some cases, PDE1 is observed in the lung, heart, and brain.

PDE2s are cGMP-stimulated PDEs that have been observed in the cerebellum, neocortex, heart, kidney, lung, pulmonary artery, and skeletal muscle. In some cases, PDE2 mediates the effects of cAMP on catecholamine secretion, participate in the regulation of aldosterone, and play a role in olfactory signal transduction.

The family of PDE3s has a high affinity for both cGMP and cAMP. PDE3 plays a role in stimulating myocardial contractility, inhibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. In some instances, isozymes of PDE3 are regulated by cAMP-dependent protein kinase, or by insulin-dependent kinases.

In some embodiments, PDE4s are specific for cAMP and are activated by cAMP-dependent phosphorylation. In some cases, PDE4s are localized to airway smooth muscle, the vascular endothelium, and all inflammatory cells.

PDE5s exert selective recognition for cGMP as a substrate and comprise two allosteric cGMP-specific binding sites. In some cases, binding of cGMP to these allosteric binding sites modulate phosphorylation of PDE5 by cGMP-dependent protein kinase. In some cases, elevated levels of PDE5 are found in vascular smooth muscle, platelets, lung, and kidney.

PDE6s, the photoreceptor cyclic nucleotide phosphodiesterases, are involved in the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which may further play a regulatory role in PDE6 function.

The PDE7 family of PDEs is cAMP specific and comprises one known member having multiple splice variants. Although mRNAs encoding PDE7s are found in skeletal muscle, heart, brain, lung, kidney, and pancreas, expression of PDE7 proteins is restricted to specific tissue types. Further, PDE7s shares a high degree of homology to the PDE4 family.

PDE8s are cAMP specific, and similar to PDE7, are closely related to the PDE4 family. In some cases, PDE8s are expressed in thyroid gland, testis, eye, liver, skeletal muscle, heart, kidney, ovary, and brain.

PDE9s are cGMP specific and closely resemble the PDE8 family of PDEs. In some cases, PDE9s are expressed in kidney, liver, lung, brain, spleen, and small intestine.

PDE10s are dual-substrate PDEs, hydrolyzing both cAMP and cGMP. In some instances, PDE10s are expressed in brain, thyroid, and testis.

PDE11s, similar to PDE10s, are dual-substrate PDEs that hydrolyze both cAMP and cGMP. In some instances, PDE11s are expressed in the skeletal muscle, brain, lung, spleen, prostate gland, and testis.

PDE12s hydrolyze cAMP and oligoadenylates (e.g., 2′,5′-oligoadenylate). In some cases, although PDE12 hydrolyzes the 2′5′ linkage, PDE12 does not exhibit activity toward 2′3′-cGAMP.

### Ecto-Nucleotide Pyrophosphatase/Phosphodiesterase

In some embodiments, the class of phosphodiesterases also comprises an ecto-nucleotide pyrophosphatase/phosphodiesterase. Ecto-nucleotide pyrophosphatase/phosphodiesterases (ENPP) or nucleotide pyrophosphatase/phosphodiesterases (NPP) are a subfamily of ectonucleotidases which hydrolyze the pyrophosphate and phosphodiester bonds of their substrates to nucleoside 5′-monophosphates. In some embodiments, ENPP (or NPP) comprises seven members, ENPP-1, ENPP-2, ENPP-3, ENPP-4, ENPP-5, ENPP-6 and ENPP-7.

The ecto-nucleotide pyrophosphatase/phosphodiesterase 1 (ENPP-1) protein (also known as PC-1) is a type II transmembrane glycoprotein comprising two identical disulfide-bonded subunits. In some instances, ENPP-1 is expressed in precursor cells and promotes osteoblast differentiation and regulates bone mineralization. In some instances, ENPP-1 negatively regulates bone mineralization by hydrolyzing extracellular nucleotide triphosphates (NTPs) to produce inorganic pyrophosphate (PPi). In some cases, expression of ENPP-1 has been observed in pancreas, kidney, bladder, and the liver. In some cases, ENPP-1 has been observed to be overexpressed in cancer cells, e.g., in breast cancer cells and glioblastoma cells.

In some embodiments, ENPP-1 has a broad specificity and cleaves a variety of substrates, including phosphodiester bonds of nucleotides and nucleotide sugars and pyrophosphate bonds of nucleotides and nucleotide sugars. In some instances, ENPP-1 functions to hydrolyze nucleoside 5′ triphosphates to their corresponding monophosphates and also hydrolyze diadenosine polyphosphates. In some cases, ENPP-1 hydrolyzes the 2′5′ linkage of cyclic nucleotides. In some cases, ENPP-1 degrades 2′3′-cGAMP, a substrate of STING.

In some embodiments, ENPP-1 comprises two N-terminal somatomedin B (SMB)-like domains (SMB1 and SMB2), a catalytic domain, and a C-terminal nuclease-like domain. In some cases, the two SMB domains are connected to the catalytic domain by a first flexible linker, while the catalytic domain is further connected to the nuclease-like domain by a second flexible linker. In some instances, the SMB domains facilitate ENPP-1 dimerization. In some cases, the catalytic domain comprises the NTP binding site. In some cases, the nuclease-like domain comprises an EF-hand motif, which binds Ca⁺² ion.

In some cases, ENPP-2 and ENPP-3 are type II transmembrane glycoproteins that share a similar architecture with ENPP-1, for example, comprising the two N-terminal SMB-like domains, a catalytic domain, and a nuclease-like domain. In some instances, ENPP-2 hydrolyzes lysophospholipids to produce lysophosphatidic acid (LPA) or sphingosylphosphorylcholine (SPC) to produce sphingosine-1 phosphate (S1P). In some cases, ENPP-3 is identified to regulate N-acetylglucosaminyltransferasc GnT-IX (GnT-Vb).

In some embodiments, ENPP-4-ENPP-7 are shorter proteins compared to ENPP-1-ENPP-3 and comprise a catalytic domain and lack the SMB-like and nuclease-like domains. ENPP-6 is a choline-specific glycerophosphodiesterase, with lysophospholipase C activity towards lysophosphatidylcholine (LPC). ENPP-7 is an alkaline sphingomyelinase (alk-SMase) with no detectable nucleotidase activity. Inhibitor of 2′3′-cGAMP Degradation Polypeptide

In some embodiments, disclosed herein are inhibitors of a 2′3′-cGAMP degradation polypeptide. In some instances, a 2′3′-cGAMP degradation polypeptide comprises a PDE protein. In some cases, a 2′3′-cGAMP degradation polypeptide comprises the ENPP-1 protein. In some cases, an inhibitor of a 2′3′-cGAMP degradation polypeptide is a small molecule inhibitor.

In some embodiments, an inhibitor of a 2′3′-cGAMP degradation polypeptide (e.g., a ENPP-1 inhibitor) described herein is a reversible inhibitor. Reversible inhibitor interacts with an enzyme with non-covalent interactions, e.g., hydrogen bonds, hydrophobic interactions, and/or ionic bonds. In some instances, a reversible inhibitor is further classified as a competitive inhibitor or an allosteric inhibitor. In competitive inhibition, both the inhibitor and the substrate compete for the same active site. In allosteric inhibition, the inhibitor binds to the enzyme at a non-active site which modulates the enzyme’s activity but does not affect binding of the substrate. In some cases, an inhibitor of a 2‘3‘-cGAMP degradation polypeptide (e.g., a ENPP-1 inhibitor) described herein is a competitive inhibitor. In other cases, an inhibitor of a 2‘3‘-cGAMP degradation polypeptide (e.g., a ENPP-1 inhibitor) described herein is an allosteric inhibitor. In some instances, the ENPP-1 inhibitor described herein is a competitive inhibitor. In other instances, the ENPP-1 inhibitor described herein is an allosteric inhibitor.

In some embodiments, an inhibitor of a 2′3′-cGAMP degradation polypeptide (e.g., a ENPP-1 inhibitor) described herein is an irreversible inhibitor. Irreversible inhibitor interacts with an enzyme with covalent interaction. In some cases, the ENPP-1 inhibitor is an irreversible inhibitor.

In some embodiments, an inhibitor of a 2‘3‘-cGAMP degradation polypeptide (e.g., a ENPP-1 inhibitor) binds to one or more domains of a PDE described herein. In some cases, a PDE inhibitor binds to one or more domains of ENPP-1. As described above, ENPP-1 comprises a catalytic domain and a nuclease-like domain. In some instances, an inhibitor of a 2′3′-cGAMP degradation polypeptide (e.g., a ENPP-1 inhibitor) binds to the catalytic domain of ENPP-1. In some cases, an inhibitor of a 2′3′-cGAMP degradation polypeptide (e.g., a ENPP-1 inhibitor) binds to the nuclease-like domain of ENPP-1.

In some cases, an inhibitor of a 2′3′-cGAMP degradation polypeptide (e.g., a ENPP-1 inhibitor) selectively binds to a region on PDE (e.g., ENPP-1) also recognized by GMP. In some cases, an inhibitor of a 2′3′-cGAMP degradation polypeptide (e.g., a ENPP-1 inhibitor) selectively binds to a region on PDE (e.g., ENPP-1) also recognized by GMP but interacts weakly with the region that is bound by AMP. In some instances, an inhibitor of a 2′3′-cGAMP degradation polypeptide (e.g., a ENPP-1 inhibitor) does not inhibit the ATP hydrolysis function of PDE. In some instances, an inhibitor of a 2′3′-cGAMP degradation polypeptide (e.g., a ENPP-1 inhibitor) weakly inhibits the ATP hydrolysis function of PDE.

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Alkyl” refers to an optionally substituted straight-chain, or optionally substituted branched-chain saturated hydrocarbon monoradical having from one to about ten carbon atoms, or from one to six carbon atoms, wherein a sp3-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, -CN, -CF₃, -OH, -OMe, -NH₂, or -NO₂. In some embodiments, the alkyl is optionally substituted with oxo, halogen, -CN, -CF₃, -OH, or -OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp2-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, -CN, -CF₃, -OH, -OMe, -NH₂, or -NO₂. In some embodiments, an alkenyl is optionally substituted with oxo, halogen, -CN, -CF₃, -OH, or -OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, -CN, -CF₃, -OH, -OMe, -NH₂, or -NO₂. In some embodiments, an alkynyl is optionally substituted with oxo, halogen, -CN, -CF₃, -OH, or -OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, -CN, -CF₃, -OH, -OMe, -NH₂, or -NO₂. In some embodiments, an alkylene is optionally substituted with oxo, halogen, -CN, -CF₃, -OH, or -OMe. In some embodiments, the alkylene is optionally substituted with halogen.

“Alkoxy” refers to a radical of the formula -ORₐ where Rₐ is an alkyl radical as defined. Unless stated otherwise specifically in the specification, an alkoxy group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, -CN, -CF₃, -OH, -OMe, -NH₂, or -NO₂. In some embodiments, an alkoxy is optionally substituted with oxo, halogen, -CN, -CF₃, -OH, or -OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, -CN, -CF₃, -OH, -OMe, -NH₂, or -NO₂. In some embodiments, an aryl is optionally substituted with halogen, methyl, ethyl, -CN, -CF₃, -OH, or -OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, -CN, -CF₃, -OH, -OMe, -NH₂, or -NO₂. In some embodiments, a cycloalkyl is optionally substituted with oxo, halogen, methyl, ethyl, -CN, -CF₃, -OH, or -OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, -CN, -CF₃, -OH, -OMe, -NH₂, or -NO₂. In some embodiments, a heterocycloalkyl is optionally substituted with oxo, halogen, methyl, ethyl, -CN, -CF₃, -OH, or -OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. -NH-, -N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl. Unless stated otherwise specifically in the specification, a Heteroalkyl is optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, -CN, -CF₃, -OH, -OMe, -NH₂, or -NO₂. In some embodiments, a heteroalkyl is optionally substituted with oxo, halogen, methyl, ethyl, -CN, -CF₃, -OH, or -OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5-to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, -CN, -CF₃, -OH, -OMe, -NH₂, or -NO₂. In some embodiments, a heteroaryl is optionally substituted with halogen, methyl, ethyl, -CN, -CF₃, -OH, or -OMe. In some embodiments, the heteroaryl is optionally substituted with halogen.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician’s assistant, an orderly or a hospice worker).

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests. For example, the term “treat” or “treating” with respect to tumor cells refers to stopping the progression of said cells, slowing down growth, inducing regression, or amelioration of symptoms associated with the presence of said cells.

The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

### Compounds

Described herein are compounds of Formula (I′), (I), (II), (III), (IV), (V), (VI), (VII), (VIII), (IX), (X), or (XI) that are ENPP-1 inhibitors. These compounds, and compositions comprising these compounds, are useful for the treatment of cancer.

Disclosed herein is a compound of Formula (I′), or a pharmaceutically acceptable salt, solvate, or stereoisomer thereof:

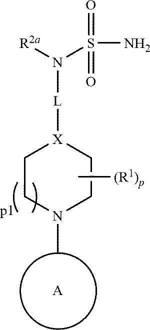

wherein
L is -(CR³R⁴)ₙ-;
X is -N- or -CH-;
Ring A is
   (a) an optionally substituted aryl or cycloalkyl;
   (b) an optionally substituted heteroaryl that is not quinazolinyl or pyrimidyl; or
   (c) an optionally substituted heterocycloalkyl; or
   (d) a ring selected from 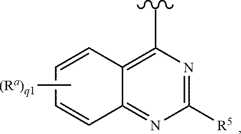 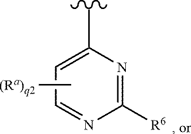 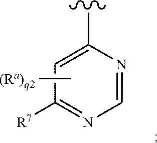
each R¹ is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

Disclosed herein is a compound of Formula (I), or a pharmaceutically acceptable salt, solvate, or stereoisomer thereof:

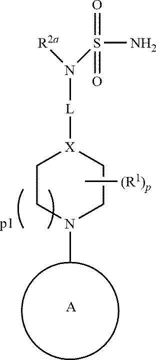

wherein
L is -(CR³R⁴)ₙ-;
X is -N- or -CH-;
Ring A is
   (a) an optionally substituted heteroaryl that is not quinazolinyl or pyrimidyl; or
   (b) an optionally substituted heterocycloalkyl; or
   (c) a ring selected from 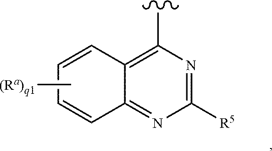 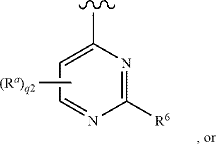 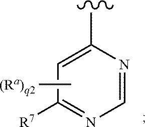
each R¹ is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R¹ on the same carbon are taken together to form an oxo;
R^{2a} is hydrogen, -SR¹¹, —S(═O)R¹⁰, —S(═O)₂R¹⁰, —S(═O)₂NR¹¹R¹², —C(═O)R¹⁰, —C(═O)NRⁿR¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R³ and R⁴ are independently hydrogen, halogen, -CN, -OR¹¹, -SR¹¹, —S(═O)R¹⁰, -NO₂, -NR¹¹R¹², —S(═O)₂R^{10,} —NR¹¹S(═O)₂R^{10,} —S(═O)₂NR¹¹R¹², —C(═O)R¹⁰, —OC(═O)R^{10,} —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³ and R⁴ on the same carbon are taken together to form an oxo;
R⁵ is halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹²,—OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ heteroalkyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁶ is hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹²,—OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl; provided that R⁶ is not substituted imidazolyl;
R⁷ is hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹²,—OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted (C₁-C₆ alkyl)aryl, optionally substituted heteroaryl, or optionally substituted (C₁-C₆ alkyl)heteroaryl; provided that R⁷ is not substituted imidazolyl;
each R^{a} is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n is 1-4;
p is 1-4;
p1 is 0 or 1;
q1 is 1-4; and
q2 is 1-2.

In some embodiments of a compound of Formula (I′) or (I), R^{2a} is hydrogen or optionally substituted C₁-C₆ alkyl. In some embodiments of a compound of Formula (I′) or (I), R^{2a} is hydrogen, C₁-C₆ alkyl, or C₁-C₆ haloalkyl. In some embodiments of a compound of Formula (I′) or (I), R^{2a} is hydrogen.

In some embodiments of a compound of Formula (I′) or (I), n is 1 or 2. In some embodiments of a compound of Formula (I′) or (I), n is 1. In some embodiments of a compound of Formula (I′) or (I), n is 2. In some embodiments of a compound of Formula (I′) or (I), n is 3. In some embodiments of a compound of Formula (I′) or (I), n is 4.

In some embodiments of a compound of Formula (I′) or (I), each R³ and R⁴ are independently hydrogen, halogen, -CN, -OR¹¹, -NR¹¹R¹², ^{_}C(═O)OR¹¹, —C(═O)NR¹¹R¹², optionally substituted C₁-C₆ alkyl, or optionally substituted C₁-C₆ heteroalkyl. In some embodiments of a compound of Formula (I′) or (I), each R³ and R⁴ are independently hydrogen, halogen, -CN, -OH, or optionally substituted C₁-C₆ alkyl. In some embodiments of a compound of Formula (I′) or (I), each R³ and R⁴ are independently hydrogen, halogen, C₁-C₆ alkyl, or C₁-C₆ haloalkyl. In some embodiments of a compound of Formula (I′) or (I), each R³ and R⁴ are independently hydrogen or halogen. In some embodiments of a compound of Formula (I′) or (I), each R³ and R⁴ are hydrogen. In some embodiments of a compound of Formula (I′) or (I), R³ and R⁴ on the same carbon are taken together to form an oxo.

In some embodiments of a compound of Formula (I′) or (I), L is -(CR³R⁴)ₙ-; n is 2; and each R³ and R⁴ are independently hydrogen or halogen.

In some embodiments of a compound of Formula (I′) or (I), X is -CH-. In some embodiments of a compound of Formula (I′) or (I), X is -N-.

In some embodiments of a compound of Formula (I′) or (I), p1 is 1. In some embodiments of a compound of Formula (I′) or (I), p1 is 0.

In some embodiments of a compound of Formula (I′) or (I), p is 1 or 2. In some embodiments of a compound of Formula (I′) or (I), p is 1. In some embodiments of a compound of Formula (I′) or (I), p is 2. In some embodiments of a compound of Formula (I′) or (I), p is 3. In some embodiments of a compound of Formula (I′) or (I), p is 4.

In some embodiments of a compound of Formula (I′) or (I), each R¹ is independently hydrogen, halogen, -CN, -OR¹¹, -NR¹¹R¹², ^{_}C(═O)OR¹¹, —C(═O)NR¹¹R¹², optionally substituted C₁-C₆ alkyl, or optionally substituted C₁-C₆ heteroalkyl. In some embodiments of a compound of Formula (I′) or (I), each R¹ is independently hydrogen, halogen, -CN, -OH, or optionally substituted C₁-C₆ alkyl. In some embodiments of a compound of Formula (I′) or (I), each R¹ is independently hydrogen, halogen, or optionally substituted C₁-C₆ alkyl. In some embodiments of a compound of Formula (I′) or (I), each R¹ is independently hydrogen, halogen, C₁-C₆ alkyl, or C₁-C₆ haloalkyl. In some embodiments of a compound of Formula (I′) or (I), each R¹ is hydrogen.

In some embodiments of a compound of Formula (I′), Ring A is aryl. In some embodiments of a compound of Formula (I′), Ring A is cycloalkyl.

In some embodiments of a compound of Formula (I′) or (I), Ring A is selected from:

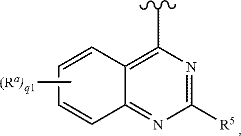

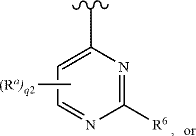

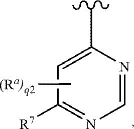

optionally substituted pyridinyl, optionally substituted pyrazinyl, optionally substituted pyridazinyl, optionally substituted pyrrolyl, optionally substituted pyrazolyl, optionally substituted imidazolyl, optionally substituted triazolyl, optionally substituted tetrazolyl, optionally substituted isoxazolyl, optionally substituted oxazolyl, optionally substituted isothiazolyl, optionally substituted thiazolyl, optionally substituted quinolinyl, optionally substituted isoquinolinyl, optionally substituted naphthyridinyl, optionally substituted cinnolinyl, optionally substituted pyridopyridazinyl, optionally substituted phthalazinyl, optionally substituted indolyl, optionally substituted pyrrolopyridinyl, optionally substituted indazolyl, optionally substituted pyrazolopyridine, optionally substituted benzotriazolyl, optionally substituted benzimidazolyl, optionally substituted pyrrolopyrimidinyl, optionally substituted pyrazolopyrimidinyl, optionally substituted triazolopyrimidinyl, optionally substituted purinyl, optionally substituted pyrrolopyridinyl, optionally substituted pyrazolopyridinyl, optionally substituted triazolopyridinyl, optionally substituted imidazopyridinyl, optionally substituted pyrrolo[2,1-f][1,2,4]triazinyl, optionally substituted pyrazolo[5,1-f][1,2,4]triazinyl, optionally substituted imidazo[5,1-f][1,2,4]triazinyl, optionally substituted imidazo[2,1-f][12,4]triazinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-b]pyridazinyl, optionally substituted pyrazolo[1,5-b]pyridazinyl, optionally substituted imidazo[1,5-b]pyridazinyl, optionally substituted imidazo[1,2-b]pyridazinyl, optionally substituted indolizinyl, optionally substituted pyrazolo[1,5-a]pyridinyl, optionally substituted imidazo[1,5-a]pyridinyl, optionally substituted imidazo[1,5-a]pyridinyl, optionally substituted imidazo[1,2-a]pyridinyl, optionally substituted pyrrolo[1,2-a][1,3,5]triaziyl, optionally substituted pyrazolo[1,5-a][1,3,5]triazinyl, optionally substituted imidazo[1,5-a][1,3,5]triazinyl, optionally substituted imidazo[1,2-a][1,3,5]triazinyl, optionally substituted pyrrolo[1,2-c]pyrimidinyl, optionally substituted pyrazolo[1,5-c]pyrimidinyl, optionally substituted imidazo[1,5-c]pyrimidinyl, optionally substituted imidazo[1,2-c]pyrimidinyl, optionally substituted pyrrolo[1,2-a]pyrazinyl, optionally substituted pyrazolo[1,5-a]pyrazinyl, optionally substituted imidazo[1,5-a]pyrazinyl, optionally substituted imidazo[1,2-a]pyrazinyl, optionally substituted pyrrolo[1,2-a]pyrimidinyl, optionally substituted pyrazolo[1,5-a]pyrimidinyl, optionally substituted imidazo[1,5-a]pyrimidinyl, optionally substituted imidazo[1,2-a]pyrimidinyl, optionally substituted tetrahydroquinazolinyl, optionally substituted dihydropyranopyrimidinyl, optionally substituted tetrahydropyridopyrimidinyl, optionally substituted tetrahydroquinolinyl, optionally substituted dihydropyranopyridinyl, optionally substituted tetrahydronaphthyridinyl, optionally substituted tetrahydroisoquinolinyl, optionally substituted dihydropyranopyridinyl, optionally substituted tetrahydronaphthyridinyl, optionally substituted dihydropurinone, optionally substituted dihydroimidazopyridinone, optionally substituted dihydrobenzoimidazolone, optionally substituted dihydropyrrolopyrimidinone, optionally substituted dihydropyrrolopyridinone, and optionally substituted indolinone.

In some embodiments of a compound of Formula (I′) or (I), Ring A is selected from:

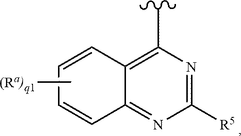

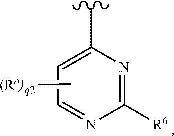

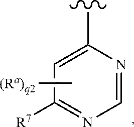

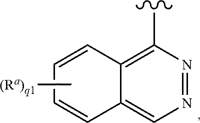

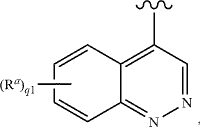

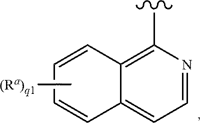

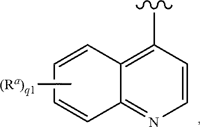

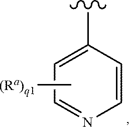

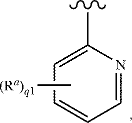

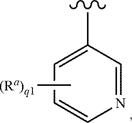

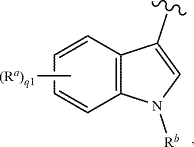

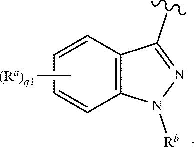

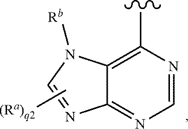

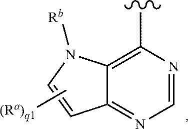

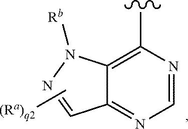

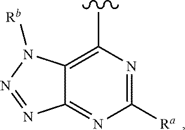

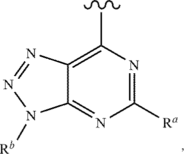

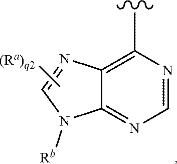

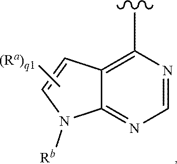

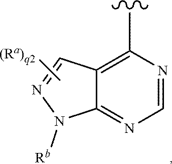

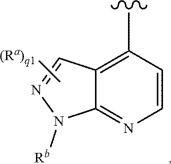

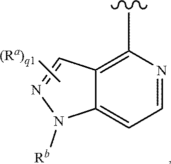

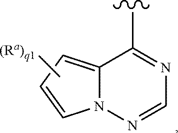

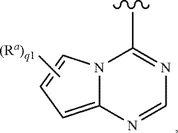

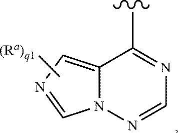

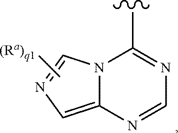

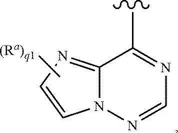

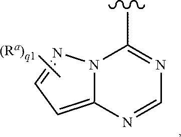

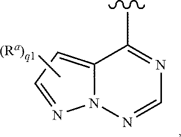

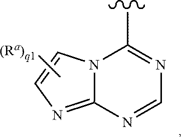

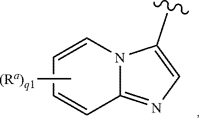

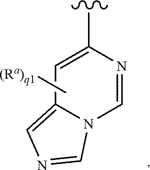

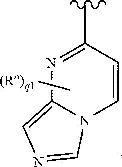

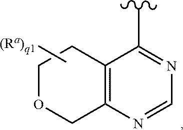

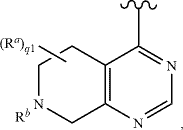

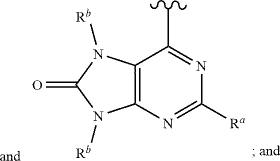

each R^{b} is independently hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl.

In some embodiments of a compound of Formula (I′) or (I), Ring A is selected from:

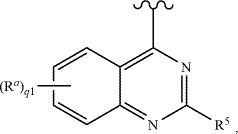

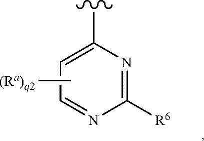

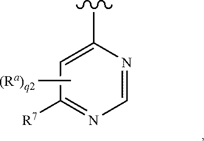

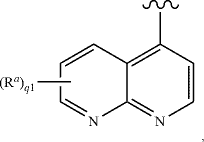

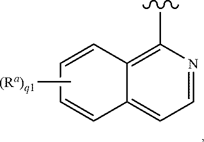

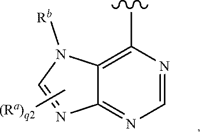

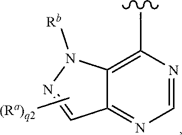

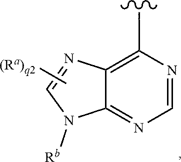

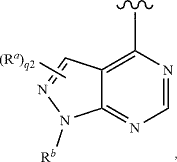

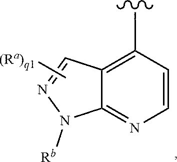

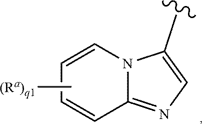

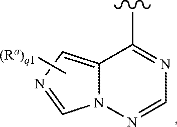

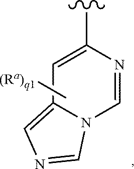

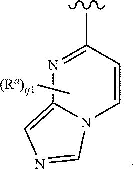

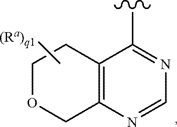

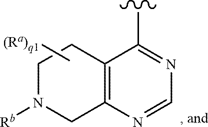

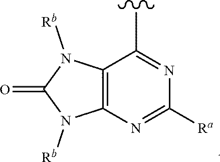

In some embodiments of a compound of Formula (I′) or (I), Ring A is

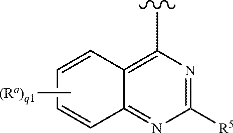

; each R^{a} is independently hydrogen, halogen, -CN, -OR¹¹, optionally substituted C₁-C₆ alkyl, or optionally substituted C₁-C₆ heteroalkyl; and q1 is 2 or 3. In some embodiments of a compound of Formula (I′) or (I), Ring A is

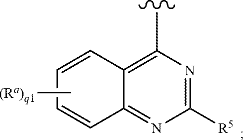

each R^{a} is -OR¹¹; and q1 is 2.

In some embodiments of a compound of Formula (I′) or (I), Ring A is

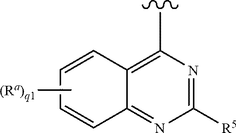

and R⁵ is halogen, -CN, -OR¹¹, -NR¹¹R¹², —C(═O)OR¹¹, —NR¹¹C(═O)R¹⁰, optionally substituted C₁-C₆ heteroalkyl, optionally substituted cycloalkyl, optionally substituted heterocycloalkyl, optionally substituted aryl, or optionally substituted heteroaryl. In some embodiments of a compound of Formula (I′) or (I), Ring A is

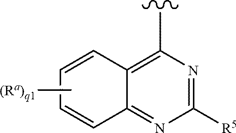

and R⁵ is -NR¹¹R¹², —NR¹¹C(═O)R¹⁰, optionally substituted aryl, or optionally substituted heteroaryl.

In some embodiments of a compound of Formula (I′) or (I), Ring A is

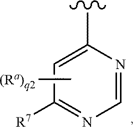

each R^{a} is independently hydrogen, halogen, -CN, -OR¹¹, optionally substituted C₁-C₆ alkyl, or optionally substituted C₁-C₆ heteroalkyl; and q2 is 1. In some embodiments of a compound of Formula (I′) or (I), Ring A is

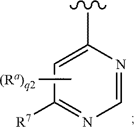

R^{a} is hydrogen or C₁-C₆ alkyl; and q2 is 1.

In some embodiments of a compound of Formula (I′) or (I), Ring A is

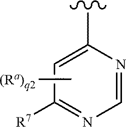

and R⁷ is hydrogen, halogen, -CN, -OR¹¹, -NR¹¹R¹², —NR¹¹C(═O)R¹⁰,—C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁷ is not substituted imidazolyl. In some embodiments of a compound of Formula (I′) or (I), Ring A is

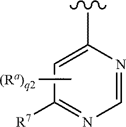

and R⁷ is optionally substituted C₁-C₆ alkyl, optionally substituted aryl, or optionally substituted heteroaryl; provided that R⁷ is not substituted imidazolyl. In some embodiments of a compound of Formula (I′) or (I), Ring A is

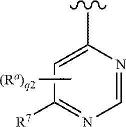

and R⁷ is optionally substituted C₁-C₆ alkyl or optionally substituted aryl.

In some embodiments of a compound of Formula (I′) or (I), Ring A is

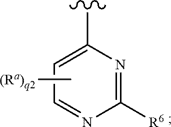

each R^{a} is independently hydrogen, halogen, -CN, -OR¹¹, optionally substituted C₁-C₆ alkyl, or optionally substituted C₁-C₆ heteroalkyl; and q2 is 1. In some embodiments of a compound of Formula (I′) or (I), Ring A is

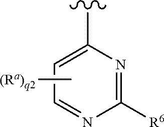

and each R^{a} is hydrogen.

In some embodiments of a compound of Formula (I′) or (I), Ring A is

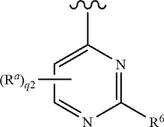

and R⁶ is hydrogen, halogen, -CN, -OR¹¹, -NR¹¹R¹², -NR¹¹C(-O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl; provided that R⁶ is not substituted imidazolyl. In some embodiments of a compound of Formula (I′) or (I), Ring A is

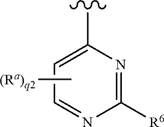

and R⁶ is hydrogen, -NR¹¹R¹², —NR¹¹C(═O)R¹⁰, or optionally substituted heteroaryl; provided that R⁶ is not substituted imidazolyl. In some embodiments of a compound of Formula (I′) or (I), Ring A is

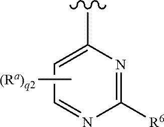

and R⁶ is hydrogen, -NR¹¹R¹², or —NR¹¹C(═O)R¹⁰.

In some embodiments of a compound of Formula (I′) or (I), Ring A is selected from:

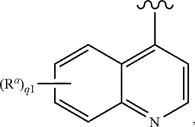

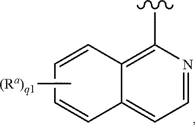

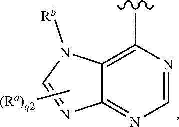

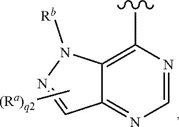

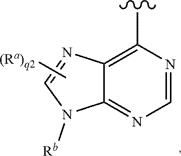

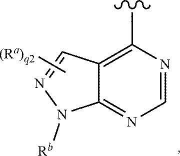

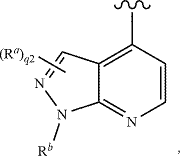

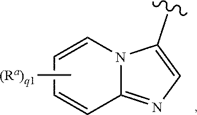

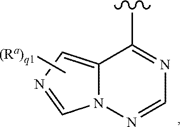

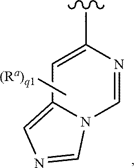

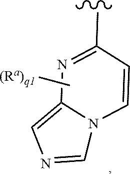

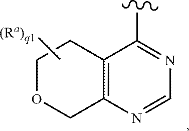

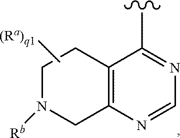

and

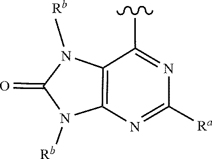

In some embodiments of a compound of Formula (I′) or (I), q1 is 1 or 2. In some embodiments of a compound of Formula (I′) or (I), q1 is 1-3. In some embodiments of a compound of Formula (I′) or (I), q1 is 1. In some embodiments of a compound of Formula (I′) or (I), q1 is 2. In some embodiments of a compound of Formula (I′) or (I), q1 is 3. In some embodiments of a compound of Formula (I′) or (I), q1 is 4. In some embodiments of a compound of Formula (I′) or (I), q2 is 1 or 2. In some embodiments of a compound of Formula (I′) or (I), q2 is 1. In some embodiments of a compound of Formula (I′) or (I), q2 is 2.

In some embodiments of a compound of Formula (I′) or (I), each R^{a} is independently hydrogen, halogen, -CN, -OR¹¹, -NR¹¹R¹², ^{_}C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl.

In some embodiments of a compound of Formula (I′) or (I), each R^{b} is independently hydrogen, optionally substituted C₁-C₆ alkyl, or optionally substituted aryl.

Also disclosed herein is a compound of Formula (II), or a pharmaceutically acceptable salt or solvate thereof:

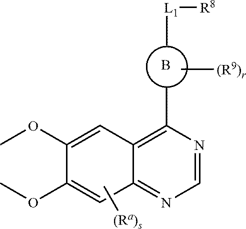

wherein
L₁ is a bond or -(CR¹³R¹⁴)ₙ₁-;
R⁸ is —S(═O)₂NH₂ or -NR^{2b}S(=O)₂NH₂;
Ring B is a bicyclic ring or a 5-membered heteroaryl ring; provided that Ring B is not triazolyl when R8 is —NHS(═O)₂NH₂;
R^{2b} is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R⁹ is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
or two R⁹ on the same carbon are taken together to form an oxo;
each R¹³ and R¹⁴ are independently hydrogen, halogen, -CN, -OR¹¹, -SR¹¹, —S(═O)R¹⁰, -NO₂, -NR¹¹R¹², —S(═O)₂R^{10,} —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R¹³ and R¹⁴ on the same carbon are taken together to form an oxo;
each R^{a} is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R^{10,} —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n1 is 1 or 2;
r is 1-4; and
s is 1-3.

In some embodiments of a compound of Formula (II), s is 1 or 2. In some embodiments of a compound of Formula (II), s is 1. In some embodiments of a compound of Formula (II), s is 2. In some embodiments of a compound of Formula (II), s is 3.

In some embodiments of a compound of Formula (II), each R^{a} is independently hydrogen, halogen, -CN, -OR¹¹, -NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl. In some embodiments of a compound of Formula (II), each R^{a} is independently hydrogen, halogen, -CN, -OH, optionally substituted C₁-C₆ alkyl, or optionally substituted C₁-C₆ heteroalkyl. In some embodiments of a compound of Formula (II), each R^{a} is independently hydrogen, halogen, or optionally substituted C₁-C₆ alkyl. In some embodiments of a compound of Formula (II), each R^{a} is independently hydrogen, halogen, C₁-C₆ alkyl, or C₁-C₆ haloalkyl.

In some embodiments of a compound of Formula (II), each R^{a} is independently hydrogen, halogen, -CN, -OR¹¹, -NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and s is 1 or 2.

In some embodiments of a compound of Formula (II), each R^{a} is hydrogen.

In some embodiments of a compound of Formula (II), n1 is 1. In some embodiments of a compound of Formula (II), n1 is 2.

In some embodiments of a compound of Formula (II), each R¹³ and R¹⁴ are independently hydrogen, halogen, -CN, -OR¹¹, -NR¹¹R¹², ^{_}C(═O)OR¹¹, —C(═O)NR¹¹R¹², optionally substituted C₁-C₆ alkyl, or optionally substituted C₁-C₆ heteroalkyl. In some embodiments of a compound of Formula (II), each R¹³ and R¹⁴ are independently hydrogen, halogen, -CN, -OH, or optionally substituted C₁-C₆ alkyl. In some embodiments of a compound of Formula (II), each R¹³ and R¹⁴ are independently hydrogen, halogen, C₁-C₆ alkyl, or C₁-C₆ haloalkyl. In some embodiments of a compound of Formula (II), each R¹³ and R¹⁴ are independently hydrogen or halogen. In some embodiments of a compound of Formula (II), each R¹³ and R¹⁴ are hydrogen. In some embodiments of a compound of Formula (II), R¹³ and R¹⁴ on the same carbon are taken together to form an oxo.

In some embodiments of a compound of Formula (II), L₁ is -(CR¹³R¹⁴)ₙ₁-; n1 is 1; and each R¹³ and R¹⁴ are independently hydrogen or halogen.

In some embodiments of a compound of Formula (II), L₁ is a bond.

In some embodiments of a compound of Formula (II), Ring B is a fused bicyclic ring. In some embodiments of a compound of Formula (II), Ring B is a spiro bicyclic ring. In some embodiments of a compound of Formula (II), Ring B is selected from

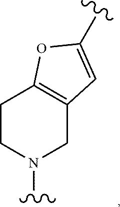

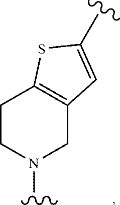

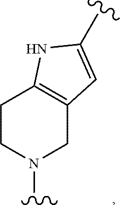

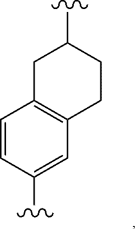

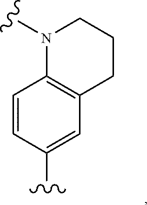

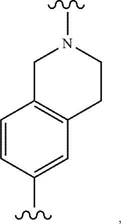

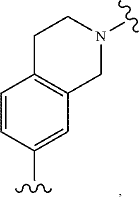

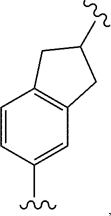

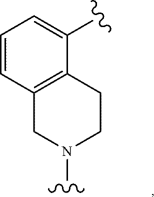

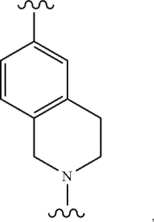

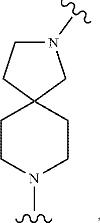

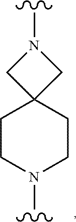

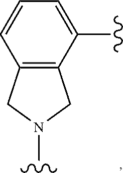

and

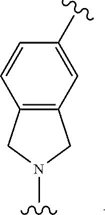

In some embodiments of a compound of Formula (II), Ring B is a 5-membered heteroaryl selected from thiophenyl, furanyl, pyrrolyl, thiazolyl, oxazolyl, imidazolyl, pyrazolyl, isoxazolyl, and isothiazolyl.

In some embodiments of a compound of Formula (II), r is 1 or 2. In some embodiments of a compound of Formula (II), r is 1. In some embodiments of a compound of Formula (II), r is 2. In some embodiments of a compound of Formula (II), r is 3. In some embodiments of a compound of Formula (II), r is 4.

In some embodiments of a compound of Formula (II), each R⁹ is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (II), each R⁹ is independently hydrogen, halogen, -CN, -OH, or optionally substituted C₁-C₆ alkyl. In some embodiments of a compound of Formula (II), each R⁹ is independently hydrogen, halogen, or optionally substituted C₁-C₆ alkyl. In some embodiments of a compound of Formula (II), each R⁹ is independently hydrogen, halogen, C₁-C₆ alkyl, or C₁-C₆ haloalkyl. In some embodiments of a compound of Formula (II), each R⁹ is hydrogen.

In some embodiments of a compound of Formula (II), R⁸ is —S(═O)₂NH₂.

In some embodiments of a compound of Formula (II), R^{2b} is hydrogen or optionally substituted C₁-C₆ alkyl. In some embodiments of a compound of Formula (II), R^{2b} is hydrogen, C₁-C₆ alkyl, or C₁-C₆ haloalkyl. In some embodiments of a compound of Formula (II), R^{2b} is hydrogen. In some embodiments of a compound of Formula (II), R⁸ is -NR^{2b}S(=O)₂NH₂; and R^{2b} is hydrogen.

Also disclosed herein is a compound of Formula (III), or a pharmaceutically acceptable salt or solvate thereof:

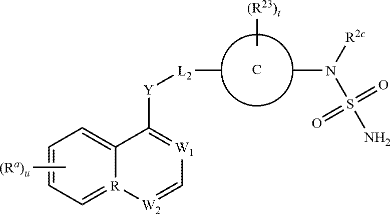

wherein
Y is —O- or—NR²⁰-;
L₂ is a bond or -(CR²¹R²²)ₙ₂-;
W₁ and W₂ are independently N or CR^{a}; provided that at least one of W₁ or W₂ is N;
Ring C is aryl, heteroaryl, cycloalkyl, or heterocycloalkyl;
each R²³ is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R^{2C} is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R²⁰ is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R²¹ and R²² are independently hydrogen, halogen, -CN, -OR¹¹, -SR¹¹, —S(═O)R¹⁰, -NO₂, -NR¹¹R¹², —S(═O)₂R^{10,} —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R²¹ and R²² on the same carbon are taken together to form an oxo;
each R^{a} is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
t is 1-4;
n2 is 1 or 2; and
u is 1-4.

In some embodiments of a compound of Formula (III), W₁ and W₂ are N.

In some embodiments of a compound of Formula (III), W₁ is N; and W₂ is CR^{a}.

In some embodiments of a compound of Formula (III), W₁ is CR^{a}; and W₂ is N.

In some embodiments of a compound of Formula (III), u is 1-3. In some embodiments of a compound of Formula (III), u is 1 or 2. In some embodiments of a compound of Formula (III), u is 1. In some embodiments of a compound of Formula (III), u is 2. In some embodiments of a compound of Formula (III), u is 3. In some embodiments of a compound of Formula (III), u is 4.

In some embodiments of a compound of Formula (III), each R^{a} is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R^{a} is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R^{a} is independently hydrogen, halogen, -OR¹¹, or optionally substituted C₁-C₆ alkyl. In some embodiments of a compound of Formula (III), each R^{a} is independently hydrogen, halogen, -OR¹¹, C₁-C₆ alkyl, or C₁-C₆ haloalkyl.

In some embodiments of a compound of Formula (III), each R^{a} is independently hydrogen, halogen, -CN, -OR¹¹, -NR¹¹R¹², —C(═O)OR¹¹, —OC(═O)OR¹¹, —C(═O)NR¹¹R¹², optionally substituted C₁-C₆ alkyl, optionally substituted C₁-C₆ heteroalkyl, optionally substituted cycloalkyl, or optionally substituted heterocycloalkyl; and u is 1-3. In some embodiments of a compound of Formula (III), each R^{a} is -OR¹¹; and u is 1 or 2.

In some embodiments of a compound of Formula (III), t is 1 or 2. In some embodiments of a compound of Formula (III), t is 1. In some embodiments of a compound of Formula (III), t is 2. In some embodiments of a compound of Formula (III), t is 3. In some embodiments of a compound of Formula (III), t is 4.

In some embodiments of a compound of Formula (III), each R²³ is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R²³ is independently hydrogen, halogen, -CN, -OH, or optionally substituted C₁-C₆ alkyl. In some embodiments of a compound of Formula (III), each R²³ is independently hydrogen, halogen, or optionally substituted C₁-C₆ alkyl. In some embodiments of a compound of Formula (III), each R²³ is independently hydrogen, halogen, C₁-C₆ alkyl, or C₁-C₆ haloalkyl. In some embodiments of a compound of Formula (III), each R²³ is hydrogen.

In some embodiments of a compound of Formula (III), Y is -NR²⁰-.

In some embodiments of a compound of Formula (III), R²⁰ is hydrogen or optionally substituted C₁-C₆ alkyl. In some embodiments of a compound of Formula (III), R²⁰ is hydrogen, C₁-C₆ alkyl, or C₁-C₆ haloalkyl. In some embodiments of a compound of Formula (III), R²⁰ is hydrogen or C₁-C₆ alkyl.

In some embodiments of a compound of Formula (III), Y is -O-.

In some embodiments of a compound of Formula (III), L₂ is a bond.

In some embodiments of a compound of Formula (III), n2 is 1. In some embodiments of a compound of Formula (III), n2 is 2.

In some embodiments of a compound of Formula (III), each R²¹ and R²² are independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (III), each R²¹ and R²² are independently hydrogen, halogen, -CN, -OH, or optionally substituted C₁-C₆ alkyl. In some embodiments of a compound of Formula (III), each R²¹ and R²² are independently hydrogen, halogen, C₁-C₆ alkyl, or C₁-C₆ haloalkyl. In some embodiments of a compound of Formula (III), each R²¹ and R²² are independently hydrogen or halogen. In some embodiments of a compound of Formula (III), each R²¹ and R²² are hydrogen. In some embodiments of a compound of Formula (III), R²¹ and R²² on the same carbon are taken together to form an oxo.

In some embodiments of a compound of Formula (III), L₂ is -(CR²¹R²²)ₙ₂-; n2 is 1 or 2; and each R²¹ and R²² are independently hydrogen or halogen.

In some embodiments of a compound of Formula (III), R^{2c} is hydrogen or optionally substituted C₁-C₆ alkyl. In some embodiments of a compound of Formula (III), R^{2c} is hydrogen, C₁-C₆ alkyl, or C₁-C₆ haloalkyl. In some embodiments of a compound of Formula (III), R^{2c} is hydrogen or C₁-C₆ alkyl. In some embodiments of a compound of Formula (III), R^{2c} is hydrogen.

In some embodiments of a compound of Formula (III), Ring C is an aryl. In some embodiments of a compound of Formula (III), Ring C is a 6-membered aryl. In some embodiments of a compound of Formula (III), Ring C is phenyl.

In some embodiments of a compound of Formula (III), Ring C is a heteroaryl. In some embodiments of a compound of Formula (III), Ring C is a 5-membered heteroaryl. In some embodiments of a compound of Formula (III), Ring C is a 5-membered heteroaryl selected from thiophenyl, furanyl, pyrrolyl, thiazolyl, oxazolyl, imidazolyl, pyrazolyl, isoxazolyl, and isothiazolyl. In some embodiments of a compound of Formula (III), Ring C is a 5-membered heteroaryl selected from thiophenyl, furanyl, thiazolyl, and oxazolyl. In some embodiments of a compound of Formula (III), Ring C is a 6-membered heteroaryl. In some embodiments of a compound of Formula (III), Ring C is pyridinyl or pyrimidyl.

In some embodiments of a compound of Formula (III), Ring C is a cycloalkyl. In some embodiments of a compound of Formula (III), Ring C is a cycloalkyl selected from cyclopropyl, cyclobuty, cyclopentyl, and cyclohexyl.

In some embodiments of a compound of Formula (III), Ring C is a heterocycloalkyl. In some embodiments of a compound of Formula (III), Ring C is a heterocycloalkyl selected from pyrrolidinyl, piperidinyl, piperazinyl, or morpholinyl.

Disclosed herein is a compound of Formula (IV), or a pharmaceutically acceptable salt, solvate, or stereoisomer thereof:

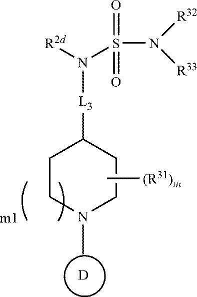

wherein
L₃ is -(CR³⁴R³⁵)ₙ₃-;
Ring D is optionally substituted heteroaryl or optionally substituted heterocycloalkyl;
each R³¹ is independently hydrogen, halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R³¹ on the same carbon are taken together to form an oxo;
R^{2d} is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R³² and R³³ are independently optionally substituted C₁-C₆ alkyl;
or R³² and R³³ taken together form an optionally substituted heterocycloalkyl;
each R³⁴ and R³⁵ are independently hydrogen, halogen, -CN, -OR¹¹, -SR¹¹, —S(═O)R¹⁰, -NO₂, -NR¹¹R¹², —S(═O)₂R^{10,} —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R³⁴ and R³⁵ on the same carbon are taken together to form an oxo;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n3 is 1-4;
m is 1-4; and
ml is 0 or 1.

In some embodiments of a compound of Formula (IV), Ring D is optionally substituted heteroaryl. In some embodiments of a compound of Formula (IV), Ring D is optionally substituted heteroaryl selected from quinolinyl, isoquinolinyl, quinazolinyl, naphthyridinyl, cinnolinyl, pyridopyridazinyl, phthalazinyl, indolyl, pyrrolopyridinyl, indazolyl, pyrazolopyridine, benzotriazolyl, benzimidazolyl, pyrrolopyrimidinyl, pyrazolopyrimidinyl, triazolopyrimidinyl, purinyl, pyrrolopyridinyl, pyrazolopyridinyl, triazolopyridinyl, and imidazopyridinyl. In some embodiments of a compound of Formula (IV), Ring D is optionally substituted heteroaryl selected from 2-pyridinyl, 3-pyridinyl, 4-pyridimidyl, 5-pyridimidyl, and 2-pyrazinyl. In some embodiments of a compound of Formula (IV), Ring D is heteroaryl optionally substituted with one, two, or three halogen, -CN, -OR¹¹, -NR¹¹R¹², —C(═O)OR¹¹, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl.

In some embodiments of a compound of Formula (IV), Ring D is optionally substituted heterocycloalkyl. In some embodiments of a compound of Formula (IV), Ring D is optionally substituted heterocycloalkyl selected from pyrrolidinyl, piperidinyl, piperazinyl, and morpholinyl. In some embodiments of a compound of Formula (IV), Ring D is optionally substituted heterocycloalkyl selected from pyrrolidinyl, piperazinyl, and morpholinyl. In some embodiments of a compound of Formula (IV), R³² and R³³ are independently optionally substituted C₁-C₆ alkyl.

In some embodiments of a compound of Formula (IV), R³² and R³³ taken together form an optionally substituted heterocycloalkyl. In some embodiments of a compound of Formula (IV), R³² and R³³ taken together form an optionally substituted heterocycloalkyl selected from pyrrolidinyl, piperidinyl, piperazinyl, and morpholinyl.

In some embodiments of a compound of Formula (IV), each R³⁴ and R³⁵ are independently hydrogen, halogen, -CN, -OR¹¹, -NR¹¹R¹², —C(═O)OR¹¹, —C(═O)NR¹¹R¹²_{,} optionally substituted C₁-C₆ alkyl, or optionally substituted C₁-C₆ heteroalkyl. In some embodiments of a compound of Formula (IV), each R³⁴ and R³⁵ are independently hydrogen, halogen, -CN, -OH, or optionally substituted C₁-C₆ alkyl. In some embodiments of a compound of Formula (IV), each R³⁴ and R³⁵ are independently hydrogen, halogen, C₁-C₆ alkyl, or C₁-C₆ haloalkyl. In some embodiments of a compound of Formula (IV), each R³⁴ and R³⁵ are independently hydrogen or halogen. In some embodiments of a compound of Formula (IV), each R³⁴ and R³⁵ are hydrogen. In some embodiments of a compound of Formula (IV), R³⁴ and R³⁵ on the same carbon are taken together to form an oxo.

In some embodiments of a compound of Formula (IV), L₃ is -(CR³⁴R³⁵)ₙ₃-; n3 is 1 or 2; and each R³⁴ and R³⁵ are independently hydrogen or halogen.

In some embodiments of a compound of Formula (IV), ml is 0. In some embodiments of a compound of Formula (IV), ml is 1.

In some embodiments of a compound of Formula (IV), R^{2d} is hydrogen or optionally substituted C₁-C₆ alkyl. In some embodiments of a compound of Formula (IV), R^{2d} is hydrogen, C₁-C₆ alkyl, or C₁-C₆ haloalkyl. In some embodiments of a compound of Formula (IV), R^{2d} is hydrogen.

In some embodiments of a compound of Formula (IV), m is 1 or 2. In some embodiments of a compound of Formula (IV), m is 1. In some embodiments of a compound of Formula (IV), m is 2. In some embodiments of a compound of Formula (IV), m is 3. In some embodiments of a compound of Formula (IV), m is 4.

In some embodiments of a compound of Formula (IV), each R³¹ is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (IV), each R³¹ is independently hydrogen, halogen, -CN, -OH, or optionally substituted C₁-C₆ alkyl. In some embodiments of a compound of Formula (IV), each R³¹ is independently hydrogen, halogen, or optionally substituted C₁-C₆ alkyl. In some embodiments of a compound of Formula (IV), each R³¹ is independently hydrogen, halogen, C₁-C₆ alkyl, or C₁-C₆ haloalkyl. In some embodiments of a compound of Formula (IV), each R³¹ is hydrogen.

In some embodiments of a compound of Formula (IV), n3 is 2-4. In some embodiments of a compound of Formula (IV), n3 is 2. In some embodiments of a compound of Formula (IV), n3 is 3. In some embodiments of a compound of Formula (IV), n3 is 4.

Disclosed herein is a compound of Formula (V), or a pharmaceutically acceptable salt, solvate, or stereoisomer thereof:

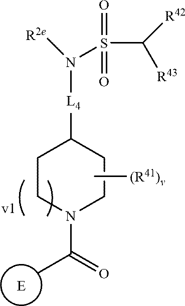

wherein
L₄ is -(CR⁴⁴R⁴⁵)ₙ₄-;
Ring E is optionally substituted cycloalkyl, optionally substituted heterocycloalkyl, optionally substituted aryl, or optionally substituted heteroaryl;
each R⁴¹ is independently hydrogen, halogen, -CN, -OR¹¹, -SR¹¹, —S(═O)R¹⁰, —NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, and optionally substituted (C₁-C₆ alkyl)heteroaryl;
or two R⁴¹ on the same carbon are taken together to form an oxo;
R^{2e} is hydrogen, -SR¹¹, —S(═O)R¹⁰, —S(═O)₂R¹⁰, —S(═O)₂NR¹¹R¹², —C(═O)R¹⁰, —C(═O)NR¹¹R¹², optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl;
R⁴² and R⁴³ are independently hydrogen, optionally substituted C₁-C₆ alkyl, optionally substituted cycloalkyl, optionally substituted heterocycloalkyl, optionally substituted aryl, or optionally substituted heteroaryl;
or R⁴² and R⁴³ taken together form an optionally substituted heterocycloalkyl;
each R⁴⁴ and R⁴⁵ are independently hydrogen, halogen, -CN, -OR¹¹, -SR¹¹, —S(═O)R¹⁰, -NO₂, -NR¹¹R¹², —S(═O)₂R^{10,} —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, or optionally substituted C₂-C₆ alkynyl;
or R⁴⁴ and R⁴⁵ on the same carbon are taken together to form an oxo;
each R¹⁰ is optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹¹ and R¹² are each independently hydrogen, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
n4 is 1-4;
v is 1-4; and
v1 is 0 or 1.

In some embodiments of a compound of Formula (V), Ring E is optionally substituted cycloalkyl. In some embodiments of a compound of Formula (V), Ring E is optionally substituted cycloalkyl selected from cyclopropyl, cyclobutyl, cyclopentyl, and cyclohexyl.

In some embodiments of a compound of Formula (V), Ring E is optionally substituted aryl. In some embodiments of a compound of Formula (V), Ring E is optionally substituted phenyl.

In some embodiments of a compound of Formula (V), Ring E is optionally substituted heteroaryl. In some embodiments of a compound of Formula (V), Ring E is optionally substituted heteroaryl selected from quinolinyl, isoquinolinyl, quinazolinyl, naphthyridinyl, cinnolinyl, pyridopyridazinyl, phthalazinyl, indolyl, pyrrolopyridinyl, indazolyl, pyrazolopyridine, benzotriazolyl, benzimidazolyl, pyrrolopyrimidinyl, pyrazolopyrimidinyl, triazolopyrimidinyl, purinyl, pyrrolopyridinyl, pyrazolopyridinyl, triazolopyridinyl, and imidazopyridinyl. In some embodiments of a compound of Formula (V), Ring E is optionally substituted heteroaryl selected from 2-pyridinyl, 3-pyridinyl, 4-pyridimidyl, 5-pyridimidyl, and 2-pyrazinyl. In some embodiments of a compound of Formula (V), Ring E is heteroaryl optionally substituted with one, two, or three halogen, -CN, -OR¹¹, -NR¹¹R¹², —C(═O)OR¹¹, —C(═O)NR¹¹R¹², optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl.

In some embodiments of a compound of Formula (V), Ring E is optionally substituted heterocycloalkyl. In some embodiments of a compound of Formula (V), Ring E is optionally substituted heterocycloalkyl selected from pyrrolidinyl, piperidinyl, piperazinyl, and morpholinyl. In some embodiments of a compound of Formula (V), Ring E is optionally substituted heterocycloalkyl selected from pyrrolidinyl, piperazinyl, and morpholinyl.

In some embodiments of a compound of Formula (V), Ring E is optionally substituted with one, two, or three halogen, -CN, -OR¹¹, -SR¹¹, —S(═O)R¹⁰, -NO₂, -NR¹¹R¹², —S(═O)₂R¹⁰, —NR¹¹S(═O)₂R¹⁰, —S(═O)₂NR¹¹R¹², —C(═O)R¹⁰, —OC(═O)R¹⁰, —C(═O)OR¹¹, —OC(═O)OR¹¹, —C(═O)NR¹¹R¹², —OC(═O)NR¹¹R¹², —NR¹¹C(═O)NR¹¹R¹², —NR¹¹C(═O)R¹⁰, —NR¹¹C(═O)OR¹¹, optionally substituted C₁-C₆ alkyl, optionally substituted C₁-C₆ heteroalkyl, optionally substituted C₂-C₆ alkenyl, optionally substituted C₂-C₆ alkynyl, optionally substituted cycloalkyl, optionally substituted (C₁-C₆ alkyl)cycloalkyl, optionally substituted heterocycloalkyl, optionally substituted (C₁-C₆ alkyl)heterocycloalkyl, optionally substituted aryl, optionally substituted (C₁-C₆ alkyl)aryl, optionally substituted heteroaryl, or optionally substituted (C₁-C₆ alkyl)heteroaryl. In some embodiments of a compound of Formula (V), Ring E is optionally substituted with one, two, or three halogen, -CN, -OR¹¹, -NR¹¹R¹², —C(═O)OR¹¹, —C(═O)NR¹¹R¹², —NR¹¹C(═O)R¹⁰, optionally substituted C₁-C₆ alkyl, optionally substituted C₁-C₆ heteroalkyl, optionally substituted cycloalkyl, optionally substituted heterocycloalkyl, optionally substituted aryl, or optionally substituted heteroaryl. In some embodiments of a compound of Formula (V), Ring E is optionally substituted with one, two, or three halogen, -OR¹¹, -NR¹¹R¹², —NR¹¹C(═O)R¹⁰, or optionally substituted C₁-C₆ alkyl. In some embodiments of a compound of Formula (V), Ring E is optionally substituted with one, two, or three halogen, -OR¹¹, -NR¹¹R¹², —NR¹¹C(═O)R¹⁰, C₁-C₆ alkyl, or C₁-C₆ haloalkyl.

In some embodiments of a compound of Formula (V), R⁴² and R⁴³ are independently hydrogen or optionally substituted C₁-C₆ alkyl.

In some embodiments of a compound of Formula (V), R⁴² and R⁴³ taken together form an optionally substituted heterocycloalkyl. In some embodiments of a compound of Formula (V), R⁴² and R⁴³ taken together form an optionally substituted heterocycloalkyl selected from pyrrolidinyl, piperidinyl, piperazinyl, and morpholinyl.

In some embodiments of a compound of Formula (V), each R⁴⁴ and R⁴⁵ are independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (V), each R⁴⁴ and R⁴⁵ are independently hydrogen, halogen, -CN, -OH, or optionally substituted C₁-C₆ alkyl. In some embodiments of a compound of Formula (V), each R⁴⁴ and R⁴⁵ are independently hydrogen, halogen, C₁-C₆ alkyl, or C₁-C₆ haloalkyl. In some embodiments of a compound of Formula (V), each R⁴⁴ and R⁴⁵ are independently hydrogen or halogen. In some embodiments of a compound of Formula (V), each R⁴⁴ and R⁴⁵ are hydrogen. In some embodiments of a compound of Formula (V), R⁴⁴ and R⁴⁵ on the same carbon are taken together to form an oxo.

In some embodiments of a compound of Formula (V), L₄ is -(CR⁴⁴R⁴⁵)ₙ₄-; n4 is 2 or 3; and each R⁴⁴ and R⁴⁵ are independently hydrogen or halogen.

In some embodiments of a compound of Formula (V), v1 is 0. In some embodiments of a compound of Formula (V), v1 is 1.

In some embodiments of a compound of Formula (V), R^{2e} is hydrogen or optionally substituted C₁-C₆ alkyl. In some embodiments of a compound of Formula (V), R^{2e} is hydrogen, C₁-C₆ alkyl, or C₁-C₆ haloalkyl. In some embodiments of a compound of Formula (V), R^{2e} is hydrogen.

In some embodiments of a compound of Formula (V), v is 1 or 2. In some embodiments of a compound of Formula (V), v is 1. In some embodiments of a compound of Formula (V), v is 2. In some embodiments of a compound of Formula (V), v is 3. In some embodiments of a compound of Formula (V), v is 4.

In some embodiments of a compound of Formula (V), each R⁴¹ is independently hydrogen, halogen, -CN, -OR¹¹, -NR¹¹R¹², —C(═O)OR¹¹, —C(═O)NR¹¹R¹², optionally substituted C₁-C₆ alkyl, or optionally substituted C₁-C₆ heteroalkyl. In some embodiments of a compound of Formula (V), each R⁴¹ is independently hydrogen, halogen, -CN, -OH, or optionally substituted C₁-C₆ alkyl. In some embodiments of a compound of Formula (V), each R⁴¹ is independently hydrogen, halogen, or optionally substituted C₁-C₆ alkyl. In some embodiments of a compound of Formula (V), each R⁴¹ is independently hydrogen, halogen, C₁-C₆ alkyl, or C₁-C₆ haloalkyl. In some embodiments of a compound of Formula (V), each R⁴¹ is hydrogen.

In some embodiments of a compound of Formula (V), n4 is 2-4. In some embodiments of a compound of Formula (V), n4 is 2. In some embodiments of a compound of Formula (V), n4 is 3. In some embodiments of a compound of Formula (V), n4 is 4.

In some embodiments of a compound of Formula (I′), (I), (II), (III), (IV), or (V), R¹⁰ is optionally substituted C₁-C₆ alkyl, optionally substituted aryl, or optionally substituted heteroaryl. In some embodiments of a compound of Formula (I′), (I), (II), (III), (IV), or (V), R¹⁰ is C₁-C₆ alkyl, C₁-C₆ haloalkyl, aryl, or heteroaryl.

In some embodiments of a compound of Formula (I′), (I), (II), (III), (IV), or (V), each R¹¹ and R¹² are each independently hydrogen, optionally substituted C₁-C₆ alkyl, optionally substituted aryl, or optionally substituted heteroaryl. In some embodiments of a compound of Formula (I′), (I), (II), (III), (IV), or (V), each R¹¹ and R¹² are each independently hydrogen, C₁-C₆ alkyl, C₁-C₆ haloalkyl, aryl, or heteroaryl.

In some embodiments of a compound of Formula (I′), (I), (II), (III), (IV), or (V), each R¹¹ is C₁-C₆ alkyl.

Also disclosed herein is a compound of Formula (VI), or a pharmaceutically acceptable salt, solvate, or stereoisomer thereof:

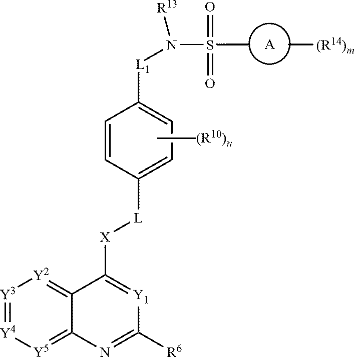

wherein
Ring A is cycloalkyl;
X is -NR⁷-, -O-, -S-, —S(═O)—, or —S(═O)₂—;
L is a bond or -CR⁸R⁹-;
L₁ is a bond or -CR¹¹R¹²-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁰ is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R⁷ and one R¹⁰ are taken together to form an optionally substituted heterocycloalkyl or an optionally substituted heteroaryl; and the remaining R¹⁰ are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹¹ and R¹² are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹³ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁴ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
m is 0-4;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

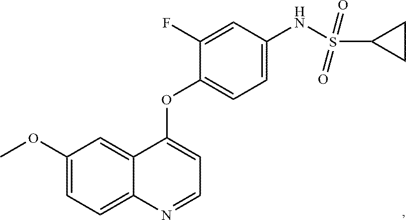

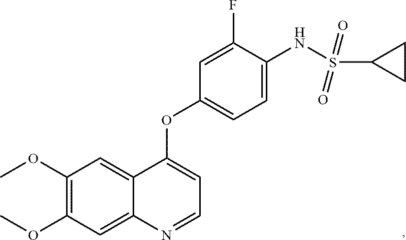

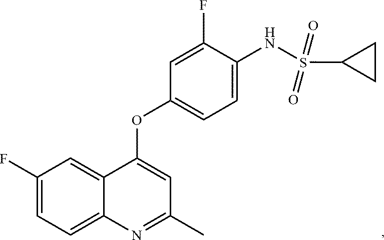

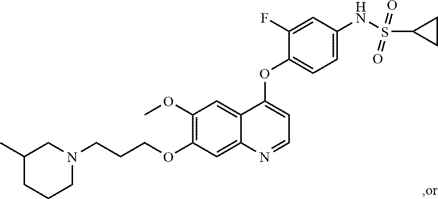

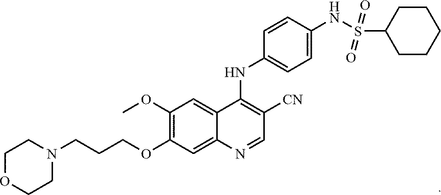

In some embodiments of a compound of Formula (VI), X is -NR⁷-.

In some embodiments of a compound of Formula (VI), R⁷ is hydrogen, C₁-C₆ alkyl, or cycloalkyl. In some embodiments of a compound of Formula (VI), R⁷ is hydrogen.

In some embodiments of a compound of Formula (VI), X is -O-. In some embodiments of a compound of Formula (VI), X is -S-.

In some embodiments of a compound of Formula (VI), L is a bond. In some embodiments of a compound of Formula (VI), L is -CR⁸R⁹-.

In some embodiments of a compound of Formula (VI), R⁸ and R⁹ are independently hydrogen, deuterium, halogen, or optionally substituted C₁-C₆ alkyl. In some embodiments of a compound of Formula (VI), R⁸ and R⁹ are independently hydrogen or C₁-C₆ alkyl.

In some embodiments of a compound of Formula (VI), each R¹⁰ is independently deuterium, halogen, -CN, -OR^{b}, -NR^{c}R^{d}, or C₁-C₆ alkyl. In some embodiments of a compound of Formula (VI), each R¹⁰ is independently halogen.

In some embodiments of a compound of Formula (VI), n is 0-2. In some embodiments of a compound of Formula (VI), n is 1. In some embodiments of a compound of Formula (VI), n is 2. In some embodiments of a compound of Formula (VI), n is 0.

In some embodiments of a compound of Formula (VI), R⁷ and one R¹⁰ are taken together to form an optionally substituted heterocycloalkyl. In some embodiments of a compound of Formula (VI), R⁷ and one R¹⁰ are taken together to form a heterocycloalkyl.

In some embodiments of a compound of Formula (VI), L₁ is a bond. In some embodiments of a compound of Formula (VI), L₁ is -CR¹¹R¹²-.

In some embodiments of a compound of Formula (VI), R¹¹ and R¹² are independently hydrogen, deuterium, halogen, or optionally substituted C₁-C₆ alkyl. In some embodiments of a compound of Formula (VI), R¹¹ and R¹² are hydrogen.

In some embodiments of a compound of Formula (VI), R¹³ is hydrogen, C₁-C₆ alkyl, cycloalkyl, or benzyl. In some embodiments of a compound of Formula (VI), R¹³ is hydrogen, C₁-C₆ alkyl, or cycloalkyl. In some embodiments of a compound of Formula (VI), R¹³ is hydrogen or C₁-C₆ alkyl. In some embodiments of a compound of Formula (VI), R¹³ is hydrogen.

In some embodiments of a compound of Formula (VI), ring A is cyclopropyl, cyclobutyl, cyclopentyl, or cyclobutyl. In some embodiments of a compound of Formula (VI), ring A is cyclopropyl.

In some embodiments of a compound of Formula (VI), each R¹⁴ is independently oxo, deuterium, halogen, -CN, -OR^{b}, -NR^{c}R^{d}, or C₁-C₆ alkyl. In some embodiments of a compound of Formula (VI), each R¹⁴ is independently oxo, deuterium, halogen, or C₁-C₆ alkyl. In some embodiments of a compound of Formula (VI), each R¹⁴ is independently deuterium, halogen, or C₁-C₆ alkyl.

In some embodiments of a compound of Formula (VI), m is 0-2. In some embodiments of a compound of Formula (VI), m is 0 or 1. In some embodiments of a compound of Formula (VI), m is 0. In some embodiments of a compound of Formula (VI), m is 1. In some embodiments of a compound of Formula (VI), m is 2.

In some embodiments of a compound of Formula (VI),

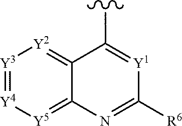

is

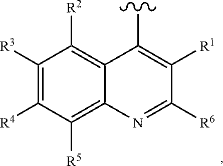

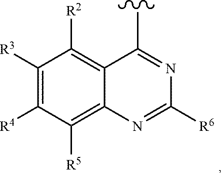

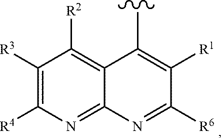

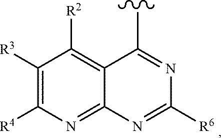

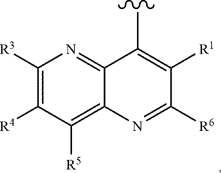

or

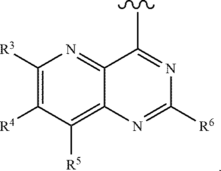

In some embodiments of a compound of Formula (VI),

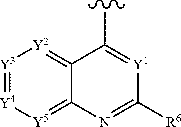

is

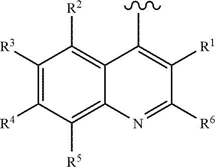

or

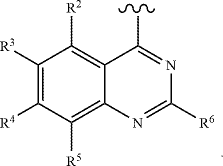

In some embodiments of a compound of Formula (VI),

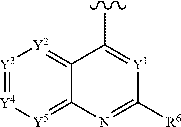

is

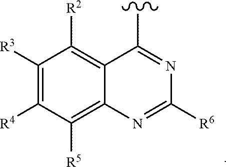

In some embodiments of a compound of Formula (VI),

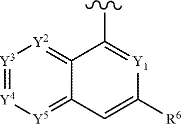

is

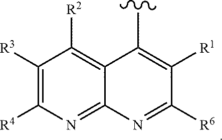

In some embodiments of a compound of Formula (VI), R¹ is hydrogen, deuterium, halogen, -CN, -OR^{b}, -NR^{c}R^{d}, or C₁-C₆ alkyl. In some embodiments of a compound of Formula (VI), R¹ is hydrogen, halogen, or -CN. In some embodiments of a compound of Formula (VI), R¹ is -CN. In some embodiments of a compound of Formula (VI), R¹ is halogen -CN. In some embodiments of a compound of Formula (VI), R¹ is hydrogen.

In some embodiments of a compound of Formula (VI), R² is hydrogen, deuterium, halogen, -CN, -OR^{b}, -NR^{c}R^{d}, or C₁-C₆ alkyl. In some embodiments of a compound of Formula (VI), R² is hydrogen, deuterium, halogen, or C₁-C₆ alkyl. In some embodiments of a compound of Formula (VI), R² is hydrogen or halogen. In some embodiments of a compound of Formula (VI), R² is hydrogen.

In some embodiments of a compound of Formula (VI), R³ is hydrogen, deuterium, halogen, -CN, -OR^{b}, -NR^{c}R^{d}, or C₁-C₆ alkyl. In some embodiments of a compound of Formula (VI), R³ is hydrogen, deuterium, halogen, -OR^{b}, or C₁-C₆ alkyl. In some embodiments of a compound of Formula (VI), R³ is hydrogen, -OR^{b}, or halogen. In some embodiments of a compound of Formula (VI), R³ is hydrogen or -OR^{b}. In some embodiments of a compound of Formula (VI), R³ is hydrogen. In some embodiments of a compound of Formula (VI), R³ is -OR^{b}.

In some embodiments of a compound of Formula (VI), R⁴ is hydrogen, deuterium, halogen, -CN, -OR^{b}, -NR^{c}R^{d}, or C₁-C₆ alkyl. In some embodiments of a compound of Formula (VI), R⁴ is hydrogen, deuterium, halogen, -OR^{b}, or C₁-C₆ alkyl. In some embodiments of a compound of Formula (VI), R⁴ is hydrogen or -OR^{b}. In some embodiments of a compound of Formula (VI), R⁴ is -OR^{b}. In some embodiments of a compound of Formula (VI), R⁴ is hydrogen.

In some embodiments of a compound of Formula (VI), R³ is OMe and R⁴ is OMe. In some embodiments of a compound of Formula (VI), R³ is OMe and R⁴ is hydrogen. In some embodiments of a compound of Formula (VI), R³ is hydrogen and R⁴ is OMe. In some embodiments of a compound of Formula (VI), R³ is hydrogen and R⁴ is OCD₃.

In some embodiments of a compound of Formula (VI), R⁵ is hydrogen, deuterium, halogen, -CN, -OR^{b}, -NR^{c}R^{d}, or C₁-C₆ alkyl. In some embodiments of a compound of Formula (VI), R⁵ is hydrogen, deuterium, halogen, or C₁-C₆ alkyl. In some embodiments of a compound of Formula (VI), R⁵ is hydrogen or halogen. In some embodiments of a compound of Formula (VI), R⁵ is hydrogen.

In some embodiments of a compound of Formula (VI), R⁶ is hydrogen, deuterium, halogen, -CN, -OR^{b}, -NR^{c}R^{d}, or C₁-C₆ alkyl. In some embodiments of a compound of Formula (VI), R⁶ is hydrogen, deuterium, halogen, or C₁-C₆ alkyl. In some embodiments of a compound of Formula (VI), R⁶ is hydrogen or halogen. In some embodiments of a compound of Formula (VI), R⁶ is hydrogen.

Also disclosed herein is a compound of Formula (VII), or a pharmaceutically acceptable salt, solvate, or stereoisomer thereof:

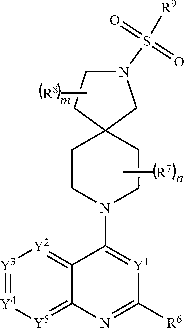

wherein
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R⁷ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-6;
each R⁸ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
m is 0-4;
R⁹ is OR¹⁰, NR¹¹R¹², optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹⁰ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹¹ and R¹² are independently hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹¹ and R¹² are taken together with the nitrogen atom to which they are attached to form an optionally substituted heterocycloalkyl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

In some embodiments of a compound of Formula (VII), each R⁷ is independently oxo, deuterium, halogen, -CN, -OR^{b}, -NR^{c}R^{d}, or C₁-C₆ alkyl. In some embodiments of a compound of Formula (VII), each R⁷ is independently oxo, deuterium, halogen, or C₁-C₆ alkyl. In some embodiments of a compound of Formula (VII), each R⁷ is independently halogen or C₁-C₆ alkyl.

In some embodiments of a compound of Formula (VII), n is 0-2. In some embodiments of a compound of Formula (VII), n is 0 or 1. In some embodiments of a compound of Formula (VII), n is 0. In some embodiments of a compound of Formula (VII), n is 1. In some embodiments of a compound of Formula (VII), n is 2.

In some embodiments of a compound of Formula (VII), each R⁸ is independently oxo, deuterium, halogen, -CN, -OR^{b}, -NR^{c}R^{d}, or C₁-C₆ alkyl. In some embodiments of a compound of Formula (VII), each R⁸ is independently oxo, deuterium, halogen, or C₁-C₆ alkyl. In some embodiments of a compound of Formula (VII), each R⁸ is independently halogen or C₁-C₆ alkyl.

In some embodiments of a compound of Formula (VII), m is 0-2. In some embodiments of a compound of Formula (VII), m is 0 or 1. In some embodiments of a compound of Formula (VII), m is 0. In some embodiments of a compound of Formula (VII), n is 1. In some embodiments of a compound of Formula (VII), m is 2.

In some embodiments of a compound of Formula (VII), R⁹ is NR¹¹R¹² or optionally substituted cycloalkyl.

In some embodiments of a compound of Formula (VII), R¹¹ and R¹² are independently hydrogen, C₁-C₆ alkyl, or cycloalkyl. In some embodiments of a compound of Formula (VII), R¹¹ and R¹² are independently hydrogen or C₁-C₆ alkyl. In some embodiments of a compound of Formula (VII), R¹¹ and R¹² are hydrogen.

In some embodiments of a compound of Formula (VII), R⁹ is cycloalkyl. In some embodiments of a compound of Formula (VII), R⁹ is cyclopropyl, cyclobutyl, cyclopentyl, or cyclobutyl. In some embodiments of a compound of Formula (VII), R⁹ is cyclopropyl.

In some embodiments of a compound of Formula (VII),

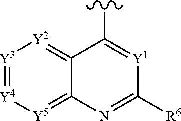

is

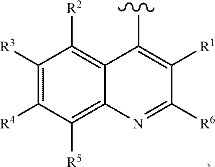

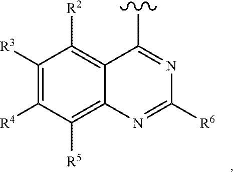

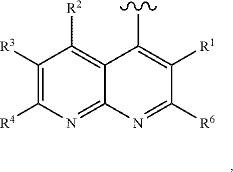

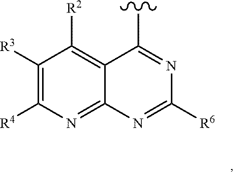

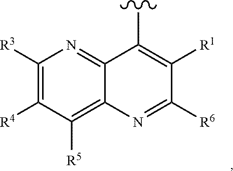

or

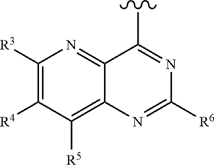

In some embodiments of a compound of Formula (VII),

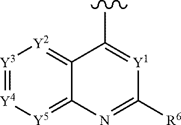

is

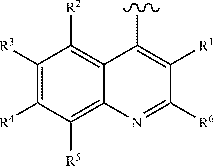

or

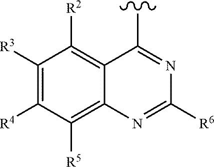

In some embodiments of a compound of Formula (VII),

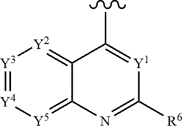

is

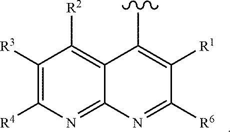

In some embodiments of a compound of Formula (VII), R¹ is hydrogen, deuterium, halogen, -CN, -OR^{b}, -NR^{c}R^{d}, or C₁-C₆ alkyl. In some embodiments of a compound of Formula (VII), R¹ is hydrogen, halogen, or -CN. In some embodiments of a compound of Formula (VII), R¹ is -CN. In some embodiments of a compound of Formula (VII), R¹ is halogen -CN. In some embodiments of a compound of Formula (VII), R¹ is hydrogen.

In some embodiments of a compound of Formula (VII), R² is hydrogen, deuterium, halogen, -CN, -OR^{b}, -NR^{c}R^{d}, or C₁-C₆ alkyl. In some embodiments of a compound of Formula (VII), R² is hydrogen, deuterium, halogen, or C₁-C₆ alkyl. In some embodiments of a compound of Formula (VII), R² is hydrogen or halogen. In some embodiments of a compound of Formula (VII), R² is hydrogen.

In some embodiments of a compound of Formula (VII), R³ is hydrogen, deuterium, halogen, -CN, -OR^{b}, -NR^{c}R^{d}, or C₁-C₆ alkyl. In some embodiments of a compound of Formula (VII), R³ is hydrogen, deuterium, halogen, -OR^{b}, or C₁-C₆ alkyl. In some embodiments of a compound of Formula (VII), R³ is hydrogen, -OR^{b}, or halogen. In some embodiments of a compound of Formula (VII), R³ is hydrogen or -OR^{b}. In some embodiments of a compound of Formula (VII), R³ is hydrogen. In some embodiments of a compound of Formula (VII), R³ is -OR^{b}.

In some embodiments of a compound of Formula (VII), R⁴ is hydrogen, deuterium, halogen, -CN, -OR^{b}, -NR^{c}R^{d}, or C₁-C₆ alkyl. In some embodiments of a compound of Formula (VII), R⁴ is hydrogen, deuterium, halogen, -OR^{b}, or C₁-C₆ alkyl. In some embodiments of a compound of Formula (VII), R⁴ is hydrogen or -OR^{b}. In some embodiments of a compound of Formula (VII), R⁴ is -OR^{b}. In some embodiments of a compound of Formula (VII), R⁴ is hydrogen.

In some embodiments of a compound of Formula (VII), R³ is OMe and R⁴ is OMe. In some embodiments of a compound of Formula (VII), R³ is OMe and R⁴ is hydrogen. In some embodiments of a compound of Formula (VII), R³ is hydrogen and R⁴ is OMe. In some embodiments of a compound of Formula (VII), R³ is hydrogen and R⁴ is OCD₃.

In some embodiments of a compound of Formula (VII), R⁵ is hydrogen, deuterium, halogen, -CN, -OR^{b}, -NR^{c}R^{d}, or C₁-C₆ alkyl. In some embodiments of a compound of Formula (VII), R⁵ is hydrogen, deuterium, halogen, or C₁-C₆ alkyl. In some embodiments of a compound of Formula (VII), R⁵ is hydrogen or halogen. In some embodiments of a compound of Formula (VII), R⁵ is hydrogen.

In some embodiments of a compound of Formula (VII), R⁶ is hydrogen, deuterium, halogen, -CN, -OR^{b}, -NR^{c}R^{d}, or C₁-C₆ alkyl. In some embodiments of a compound of Formula (VII), R⁶ is hydrogen, deuterium, halogen, or C₁-C₆ alkyl. In some embodiments of a compound of Formula (VII), R⁶ is hydrogen or halogen. In some embodiments of a compound of Formula (VII), R⁶ is hydrogen.

Also disclosed herein is a compound of Formula (VIII), or a pharmaceutically acceptable salt, solvate, or stereoisomer thereof:

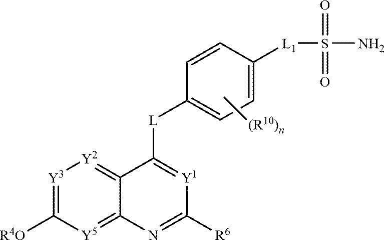

wherein
L is a bond, -O-, -S-, —S(═O)—, —S(═O)₂—, -O(CR⁸R⁹)-, -S(CR⁸R⁹)-, or -NR⁷(CR⁸R⁹)-;
L₁ is a bond, -O-, or -CR¹¹R¹²-;
Y¹ is -N- or -CR¹-;
Y² is -N- or -CR²-;
Y³ is -N- or -CR³-;
Y⁵ is -N- or -CR⁵-;
R¹, R², R³, and R⁵ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁴ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁶ is hydrogen, deuterium, halogen, -CN, -OR^{b}, -NO₂, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹⁰ is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹¹ and R¹² are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

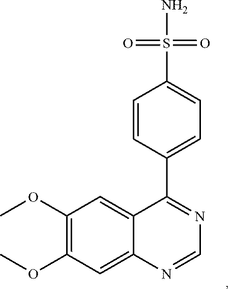

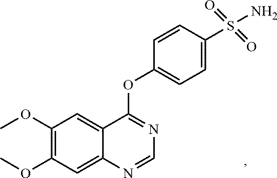

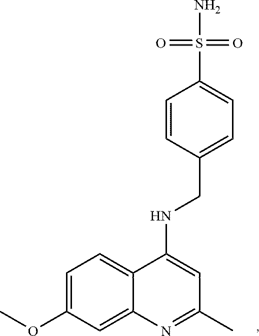

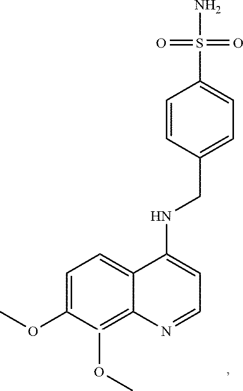

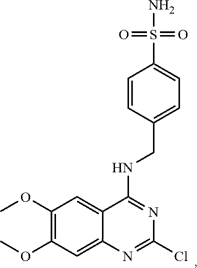

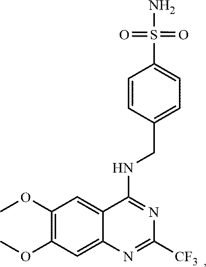

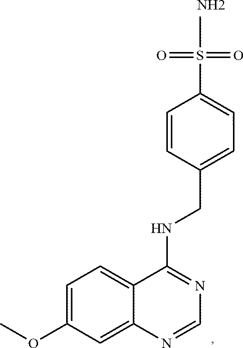

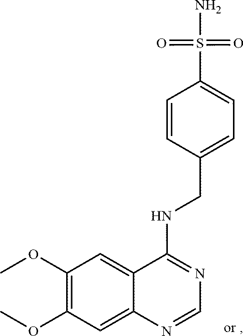

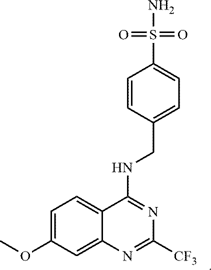

In some embodiments of a compound of Formula (VIII), L is a bond, -O-, -S-, —S(═O)—, —S(═O)₂—, -O(CR⁸R⁹)-, or -S(CR⁸R⁹)-. In some embodiments of a compound of Formula (VIII), L is -O-, -S-, —S(═O)—, —S(═O)₂—, -O(CR⁸R⁹)-, or -S(CR⁸R⁹)-. In some embodiments of a compound of Formula (VIII), L is a bond, -O-, or -O(CR⁸R⁹)-. In some embodiments of a compound of Formula (VIII), L is -O-

In some embodiments of a compound of Formula (VIII), each R¹⁰ is independently deuterium, halogen, -CN, -OR^{b}, -NR^{c}R^{d}, or C₁-C₆ alkyl. In some embodiments of a compound of Formula (VIII), each R¹⁰ is independently deuterium, halogen, or C₁-C₆ alkyl. In some embodiments of a compound of Formula (VIII), each R¹⁰ is independently halogen or C₁-C₆ alkyl.

In some embodiments of a compound of Formula (VIII), n is 0-2. In some embodiments of a compound of Formula (VIII), n is 0 or 1. In some embodiments of a compound of Formula (VIII), n is 0. In some embodiments of a compound of Formula (VIII), n is 1. In some embodiments of a compound of Formula (VIII), n is 2.

In some embodiments of a compound of Formula (VIII), L₁ is a bond. In some embodiments of a compound of Formula (VIII), L₁ is -CR¹¹R¹²-.

In some embodiments of a compound of Formula (VIII), R¹¹ and R¹² are independently hydrogen, deuterium, halogen, or C₁-C₆ alkyl. In some embodiments of a compound of Formula (VIII), R¹¹ and R¹² are independently hydrogen, halogen, or C₁-C₆ alkyl. In some embodiments of a compound of Formula (VIII), R¹¹ and R¹² are hydrogen.

In some embodiments of a compound of Formula (VIII),

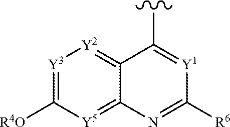

is

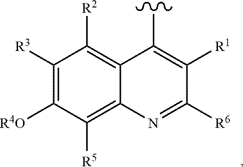

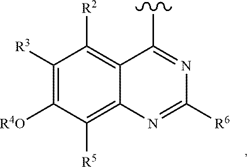

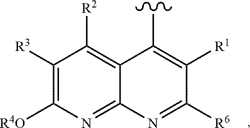

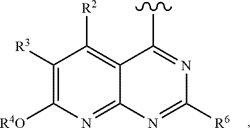

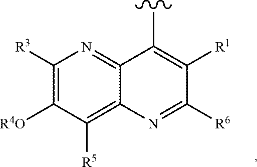

or

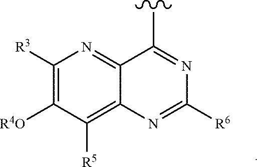

In some embodiments of a compound of Formula (VIII),

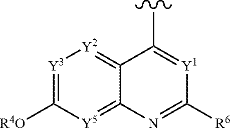

is

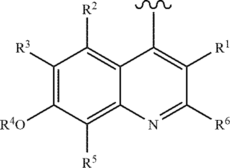

or

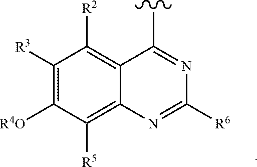

In some embodiments of a compound of Formula (VIII),

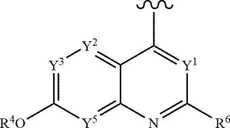

is

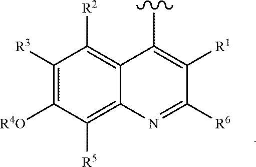

In some embodiments of a compound of Formula (VIII),

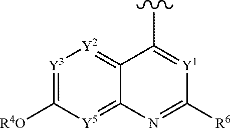

is

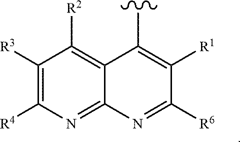

In some embodiments of a compound of Formula (VIII), R¹ is hydrogen, deuterium, halogen, -CN, -OR^{b}, -NR^{c}R^{d}, or C₁-C₆ alkyl. In some embodiments of a compound of Formula (VIII), R¹ is hydrogen, halogen, or -CN. In some embodiments of a compound of Formula (VIII), R¹ is -CN. In some embodiments of a compound of Formula (VIII), R¹ is halogen -CN. In some embodiments of a compound of Formula (VIII), R¹ is hydrogen.

In some embodiments of a compound of Formula (VIII), R² is hydrogen, deuterium, halogen, -CN, -OR^{b}, -NR^{c}R^{d}, or C₁-C₆ alkyl. In some embodiments of a compound of Formula (VIII), R² is hydrogen, deuterium, halogen, or C₁-C₆ alkyl. In some embodiments of a compound of Formula (VIII), R² is hydrogen or halogen. In some embodiments of a compound of Formula (VIII), R² is hydrogen.

In some embodiments of a compound of Formula (VIII), R³ is hydrogen, deuterium, halogen, -CN, -OR^{b}, -NR^{c}R^{d}, or C₁-C₆ alkyl. In some embodiments of a compound of Formula (VIII), R³ is hydrogen, deuterium, halogen, -OR^{b}, or C₁-C₆ alkyl. In some embodiments of a compound of Formula (VIII), R³ is hydrogen, -OR^{b}, or halogen. In some embodiments of a compound of Formula (VIII), R³ is hydrogen or -OR^{b}. In some embodiments of a compound of Formula (VIII), R³ is hydrogen. In some embodiments of a compound of Formula (VIII), R³ is -OR^{b}.

In some embodiments of a compound of Formula (VIII), R⁴ is hydrogen, C₁-C₆ alkyl, C₁-C₆ haloalkyl, or cycloalkyl. In some embodiments of a compound of Formula (VIII), R⁴ is hydrogen, C₁-C₆ alkyl, or C₁-C₆ haloalkyl. In some embodiments of a compound of Formula (VIII), R⁴ is C₁-C₆ alkyl or C₁-C₆ haloalkyl. In some embodiments of a compound of Formula (VIII), R⁴ is C₁-C₆ alkyl.

In some embodiments of a compound of Formula (VIII), R⁵ is hydrogen, deuterium, halogen, -CN, -OR^{b}, -NR^{c}R^{d}, or C₁-C₆ alkyl. In some embodiments of a compound of Formula (VIII), R⁵ is hydrogen, deuterium, halogen, or C₁-C₆ alkyl. In some embodiments of a compound of Formula (VIII), R⁵ is hydrogen or halogen. In some embodiments of a compound of Formula (VIII), R⁵ is hydrogen.

In some embodiments of a compound of Formula (VIII), R⁶ is hydrogen, deuterium, halogen, -CN, -OR^{b}, or C₁-C₆ alkyl. In some embodiments of a compound of Formula (VIII), R⁶ is hydrogen, deuterium, halogen, or C₁-C₆ alkyl. In some embodiments of a compound of Formula (VIII), R⁶ is hydrogen or halogen. In some embodiments of a compound of Formula (VIII), R⁶ is hydrogen.

Also disclosed herein is a compound of Formula (IX), or a pharmaceutically acceptable salt, solvate, or stereoisomer thereof:

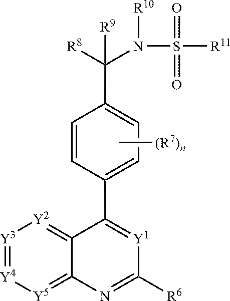

wherein
Y¹ is -N- or -CR¹-;
Y² is -N- or -CR²-;
Y³ is -N- or -CR³-;
Y⁴ is -N- or -CR⁴-.,
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹⁰ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl
or R¹⁰ and one R⁷ are taken together to form an optionally substituted heterocycloalkyl; and the remaining R¹⁰ are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹¹ is -OR¹², NR¹³R¹⁴, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹² is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹³ and R¹⁴ are independently hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R¹³ and R¹⁴ are taken together with the nitrogen atom to which they are attached to form an optionally substituted heterocycloalkyl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

In some embodiments of a compound of Formula (IX), each R⁷ is independently deuterium, halogen, -CN, -OR^{b}, -NR^{c}R^{d}, or C₁-C₆ alkyl. In some embodiments of a compound of Formula (IX), each R⁷ is independently deuterium, halogen, or C₁-C₆ alkyl. In some embodiments of a compound of Formula (IX), each R⁷ is independently halogen or C₁-C₆ alkyl.

In some embodiments of a compound of Formula (IX), n is 0-2. In some embodiments of a compound of Formula (IX), n is 0 or 1. In some embodiments of a compound of Formula (IX), n is 0. In some embodiments of a compound of Formula (IX), n is 1. In some embodiments of a compound of Formula (IX), n is 2.

In some embodiments of a compound of Formula (IX), R⁸ and R⁹ are independently hydrogen, deuterium, halogen, or optionally substituted C₁-C₆ alkyl. In some embodiments of a compound of Formula (IX), R⁸ and R⁹ are independently hydrogen, halogen, or C₁-C₆ alkyl. In some embodiments of a compound of Formula (IX), R⁸ and R⁹ are hydrogen.

In some embodiments of a compound of Formula (IX), R¹⁰ is hydrogen, C₁-C₆ alkyl, cycloalkyl, or benzyl. In some embodiments of a compound of Formula (IX), R¹⁰ is hydrogen, C₁-C₆ alkyl, or cycloalkyl. In some embodiments of a compound of Formula (IX), R¹⁰ is hydrogen or C₁-C₆ alkyl. In some embodiments of a compound of Formula (IX), R¹⁰ is hydrogen.

In some embodiments of a compound of Formula (IX), R¹⁰ and one R⁷ are taken together to form an optionally substituted heterocycloalkyl. In some embodiments of a compound of Formula (IX), R¹⁰ and one R⁷ are taken together to form a heterocycloalkyl.

In some embodiments of a compound of Formula (IX), R¹¹ is NR¹³R¹⁴ or optionally substituted cycloalkyl. In some embodiments of a compound of Formula (IX), R¹¹ is NR¹³R¹⁴ or cycloalkyl.

In some embodiments of a compound of Formula (IX), R¹³ and R¹⁴ are independently hydrogen, C₁-C₆ alkyl, or cycloalkyl. In some embodiments of a compound of Formula (IX), R¹³ and R¹⁴ are independently hydrogen or C₁-C₆ alkyl. In some embodiments of a compound of Formula (IX), R¹³ and R¹⁴ are hydrogen.

In some embodiments of a compound of Formula (IX), R¹¹ is cycloalkyl. In some embodiments of a compound of Formula (IX), R¹¹ is cyclopropyl, cyclobutyl, cyclopentyl, or cyclohexyl. In some embodiments of a compound of Formula (IX), R¹¹ is cyclopropyl.

In some embodiments of a compound of Formula (IX),

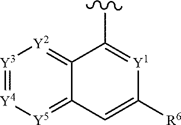

is

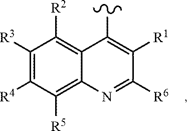

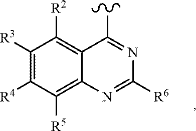

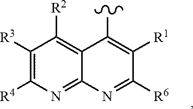

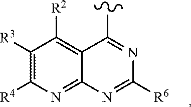

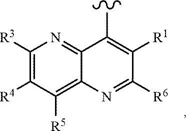

or

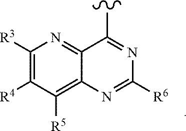

In some embodiments of a compound of Formula (IX),

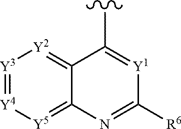

is

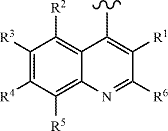

or

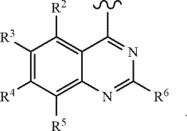

In some embodiments of a compound of Formula (IX),

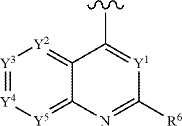

is

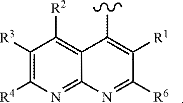

In some embodiments of a compound of Formula (IX), R¹ is hydrogen, deuterium, halogen, -CN, -OR^{b}, -NR^{c}R^{d}, or C₁-C₆ alkyl. In some embodiments of a compound of Formula (IX), R¹ is hydrogen, halogen, or -CN. In some embodiments of a compound of Formula (IX), R¹ is -CN. In some embodiments of a compound of Formula (IX), R¹ is halogen -CN. In some embodiments of a compound of Formula (IX), R¹ is hydrogen.

In some embodiments of a compound of Formula (IX), R² is hydrogen, deuterium, halogen, -CN, -OR^{b}, -NR^{c}R^{d}, or C₁-C₆ alkyl. In some embodiments of a compound of Formula (IX), R² is hydrogen, deuterium, halogen, or C₁-C₆ alkyl. In some embodiments of a compound of Formula (IX), R² is hydrogen or halogen. In some embodiments of a compound of Formula (IX), R² is hydrogen.

In some embodiments of a compound of Formula (IX), R³ is hydrogen, deuterium, halogen, -CN, -OR^{b}, -NR^{c}R^{d}, or C₁-C₆ alkyl. In some embodiments of a compound of Formula (IX), R³ is hydrogen, deuterium, halogen, -OR^{b}, or C₁-C₆ alkyl. In some embodiments of a compound of Formula (IX), R³ is hydrogen, -OR^{b}, or halogen. In some embodiments of a compound of Formula (IX), R³ is hydrogen or -OR^{b}. In some embodiments of a compound of Formula (IX), R³ is hydrogen. In some embodiments of a compound of Formula (IX), R³ is -OR^{b}.

In some embodiments of a compound of Formula (IX), R⁴ is hydrogen, deuterium, halogen, -CN, -OR^{b}, -NR^{c}R^{d}, or C₁-C₆ alkyl. In some embodiments of a compound of Formula (IX), R⁴ is hydrogen, deuterium, halogen, -OR^{b}, or C₁-C₆ alkyl. In some embodiments of a compound of Formula (IX), R⁴ is hydrogen or -OR^{b}. In some embodiments of a compound of Formula (IX), R⁴ is -OR^{b}. In some embodiments of a compound of Formula (IX), R⁴ is hydrogen.

In some embodiments of a compound of Formula (IX), R³ is OMe and R⁴ is OMe. In some embodiments of a compound of Formula (IX), R³ is OMe and R⁴ is hydrogen. In some embodiments of a compound of Formula (IX), R³ is hydrogen and R⁴ is OMe. In some embodiments of a compound of Formula (IX), R³ is hydrogen and R⁴ is OCD₃.

In some embodiments of a compound of Formula (IX), R⁵ is hydrogen, deuterium, halogen, -CN, -OR^{b}, -NR^{c}R^{d}, or C₁-C₆ alkyl. In some embodiments of a compound of Formula (IX), R⁵ is hydrogen, deuterium, halogen, or C₁-C₆ alkyl. In some embodiments of a compound of Formula (IX), R⁵ is hydrogen or halogen. In some embodiments of a compound of Formula (IX), R⁵ is hydrogen.

In some embodiments of a compound of Formula (IX), R⁶ is hydrogen, deuterium, halogen, -CN, -OR^{b}, -NR^{c}R^{d}, or C₁-C₆ alkyl. In some embodiments of a compound of Formula (IX), R⁶ is hydrogen, deuterium, halogen, or C₁-C₆ alkyl. In some embodiments of a compound of Formula (IX), R⁶ is hydrogen or halogen. In some embodiments of a compound of Formula (IX), R⁶ is hydrogen.

Also disclosed herein is a compound of Formula (X), or a pharmaceutically acceptable salt, solvate, or stereoisomer thereof:

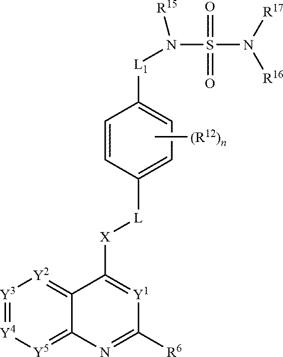

wherein
X is -NR7-, -O-, -S-, —S(═O)—, —S(═O)₂—, or -CR⁸R⁹-;
L is a bond or -CR¹⁰R¹¹-;
L₁ is a bond or -CR¹³R¹⁴-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d,} optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, or optionally substituted heterocycloalkyl;
R¹⁰ and R¹¹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹² is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R⁷ and one R¹² are taken together to form an optionally substituted heterocycloalkyl or an optionally substituted heteroaryl; and the remaining R¹² are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl
R¹³ and R¹⁴ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹⁵ is hydrogen, deuterium, C₁-C₆ alkyl, C₁-C₆ haloalkyl, optionally substituted C₂-C₆ alkenyl, optionally substituted C₂-C₆ alkynyl, optionally substituted cycloalkyl, or optionally substituted heterocycloalkyl;
R¹⁶ and R¹⁷ are independently hydrogen, —C(═O)R^{a}, —C(═O)OR^{b}, —C(═O)NR^{c}R^{d}, C₁C₆ alkyl, C₁-C₆ haloalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not:

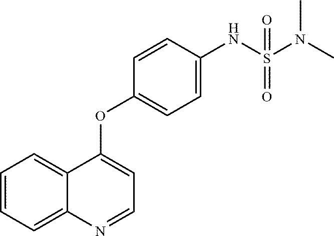 [SMILES: CN(C)S(=O)(=O)Nc1ccc(Oc2ccnc3ccccc23)cc1]

or

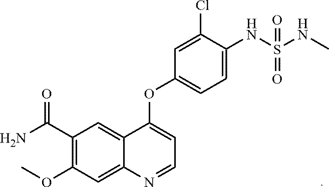

In some embodiments of a compound of Formula (X), X is -NR⁷-.

In some embodiments of a compound of Formula (X), R⁷ is hydrogen or C₁-C₆ alkyl. In some embodiments of a compound of Formula (X), R⁷ is hydrogen.

In some embodiments of a compound of Formula (X), X is -O-.

In some embodiments of a compound of Formula (X), L is a bond. In some embodiments of a compound of Formula (X), L is -CR⁸R⁹-.

In some embodiments of a compound of Formula (X), R⁸ and R⁹ are independently hydrogen, deuterium, halogen, or optionally substituted C₁-C₆ alkyl. In some embodiments of a compound of Formula (X), R⁸ and R⁹ are independently hydrogen, deuterium, halogen, or C₁-C₆ alkyl. In some embodiments of a compound of Formula (X), R⁸ and R⁹ are independently hydrogen, halogen, or C₁-C₆ alkyl. In some embodiments of a compound of Formula (X), R⁸ and R⁹ are independently hydrogen or C₁-C₆ alkyl. In some embodiments of a compound of Formula (X), R⁸ and R⁹ are hydrogen.

In some embodiments of a compound of Formula (X), each R¹² is independently deuterium, halogen, -CN, -OR^{b}, -NR^{c}R^{d}, or C₁-C₆ alkyl. In some embodiments of a compound of Formula (X), each R¹² is independently deuterium, halogen, or C₁-C₆ alkyl. In some embodiments of a compound of Formula (X), each R¹² is independently halogen or C₁-C₆ alkyl. In some embodiments of a compound of Formula (X), each R¹² is independently halogen.

In some embodiments of a compound of Formula (X), n is 0-2. In some embodiments of a compound of Formula (X), n is 0 or 1. In some embodiments of a compound of Formula (X), n is 0. In some embodiments of a compound of Formula (X), n is 1. In some embodiments of a compound of Formula (X), n is 2.

In some embodiments of a compound of Formula (X), R⁷ and one R¹² are taken together to form an optionally substituted heterocycloalkyl. In some embodiments of a compound of Formula (X), R⁷ and one R¹² are taken together to form a heterocycloalkyl.

In some embodiments of a compound of Formula (X), L₁ is a bond. In some embodiments of a compound of Formula (X), L₁ is -CR¹³R¹⁴-.

In some embodiments of a compound of Formula (X), R¹³ and R¹⁴ are independently hydrogen, deuterium, halogen, or optionally substituted C₁-C₆ alkyl. In some embodiments of a compound of Formula (X), R¹³ and R¹⁴ are independently hydrogen, deuterium, halogen, or C₁-C₆ alkyl. In some embodiments of a compound of Formula (X), R¹³ and R¹⁴ are independently hydrogen, halogen, or C₁-C₆ alkyl. In some embodiments of a compound of Formula (X), R¹³ and R¹⁴ are hydrogen.

In some embodiments of a compound of Formula (X), R¹⁵ is hydrogen, C₁-C₆ alkyl, cycloalkyl, or benzyl. In some embodiments of a compound of Formula (X), R¹⁵ is hydrogen, C₁-C₆ alkyl, or cycloalkyl. In some embodiments of a compound of Formula (X), R¹⁵ is hydrogen or C₁-C₆ alkyl. In some embodiments of a compound of Formula (X), R¹⁵ is hydrogen.

In some embodiments of a compound of Formula (X), R¹⁶ and R¹⁷ are independently hydrogen, C₁-C₆ alkyl, or cycloalkyl. In some embodiments of a compound of Formula (X), R¹⁶ and R¹⁷ are independently hydrogen or cycloalkyl. In some embodiments of a compound of Formula (X), R¹⁶ and R¹⁷ are independently hydrogen or C₁-C₆ alkyl. In some embodiments of a compound of Formula (X), R¹⁶ and R¹⁷ are hydrogen.

In some embodiments of a compound of Formula (X),

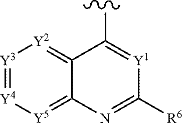

is

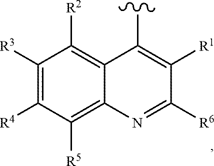

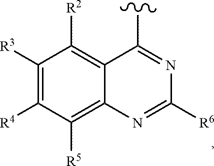

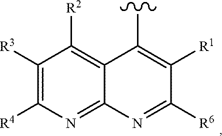

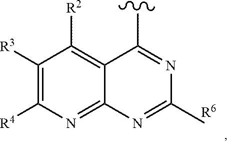

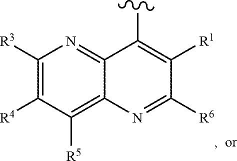

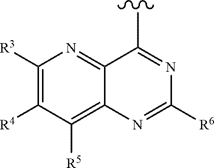

In some embodiments of a compound of Formula (X),

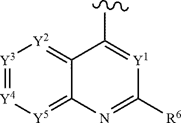

is

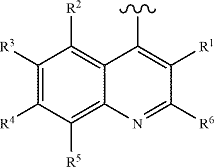

or

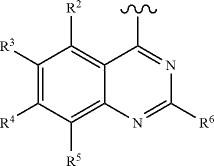

In some embodiments of a compound of Formula (X),

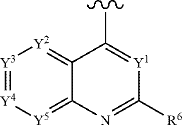

is

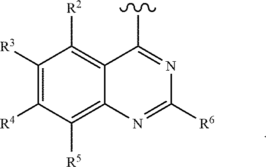

In some embodiments of a compound of Formula (X),

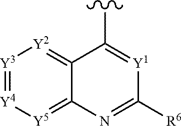

is

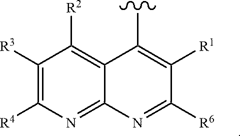

In some embodiments of a compound of Formula (X), R¹ is hydrogen, deuterium, halogen, -CN, -OR^{b}, -NR^{c}R^{d}, or C₁-C₆ alkyl. In some embodiments of a compound of Formula (X), R¹ is hydrogen, halogen, or -CN. In some embodiments of a compound of Formula (X), R¹ is -CN. In some embodiments of a compound of Formula (X), R¹ is halogen -CN. In some embodiments of a compound of Formula (X), R¹ is hydrogen.

In some embodiments of a compound of Formula (X), R² is hydrogen, deuterium, halogen, -CN, -OR^{b}, -NR^{c}R^{d}, or C₁-C₆ alkyl. In some embodiments of a compound of Formula (X), R² is hydrogen, deuterium, halogen, or C₁-C₆ alkyl. In some embodiments of a compound of Formula (X), R² is hydrogen or halogen. In some embodiments of a compound of Formula (X), R² is hydrogen.

In some embodiments of a compound of Formula (X), R³ is hydrogen, deuterium, halogen, -CN, -OR^{b}, -NR^{c}R^{d}, or C₁-C₆ alkyl. In some embodiments of a compound of Formula (X), R³ is hydrogen, deuterium, halogen, -OR^{b}, or C₁-C₆ alkyl. In some embodiments of a compound of Formula (X), R³ is hydrogen, -OR^{b}, or halogen. In some embodiments of a compound of Formula (X), R³ is hydrogen or -OR^{b}. In some embodiments of a compound of Formula (X), R³ is hydrogen. In some embodiments of a compound of Formula (X), R³ is -OR^{b}.

In some embodiments of a compound of Formula (X), R⁴ is hydrogen, deuterium, halogen, -CN, -OR^{b}, -NR^{c}R^{d}, or C₁-C₆ alkyl. In some embodiments of a compound of Formula (X), R⁴ is hydrogen, deuterium, halogen, -OR^{b}, or C₁-C₆ alkyl. In some embodiments of a compound of Formula (X), R⁴ is hydrogen or -OR^{b}. In some embodiments of a compound of Formula (X), R⁴ is -OR^{b}. In some embodiments of a compound of Formula (X), R⁴ is hydrogen.

In some embodiments of a compound of Formula (X), R³ is OMe and R⁴ is OMe. In some embodiments of a compound of Formula (X), R³ is OMe and R⁴ is hydrogen. In some embodiments of a compound of Formula (X), R³ is hydrogen and R⁴ is OMe. In some embodiments of a compound of Formula (X), R³ is hydrogen and R⁴ is OCD₃.

In some embodiments of a compound of Formula (X), R⁵ is hydrogen, deuterium, halogen, -CN, -OR^{b}, -NR^{c}R^{d}, or C₁-C₆ alkyl. In some embodiments of a compound of Formula (X), R⁵ is hydrogen, deuterium, halogen, or C₁-C₆ alkyl. In some embodiments of a compound of Formula (X), R⁵ is hydrogen or halogen. In some embodiments of a compound of Formula (X), R⁵ is hydrogen.

In some embodiments of a compound of Formula (X), R⁶ is hydrogen, deuterium, halogen, -CN, -OR^{b}, X, or C₁-C₆ alkyl. In some embodiments of a compound of Formula (X), R⁶ is hydrogen, deuterium, halogen, or C₁-C₆ alkyl. In some embodiments of a compound of Formula (X), R⁶ is hydrogen or halogen. In some embodiments of a compound of Formula (X), R⁶ is hydrogen.

Also disclosed herein is a compound of Formula (XI), or a pharmaceutically acceptable salt, solvate, or stereoisomer thereof:

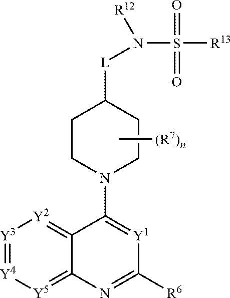

wherein
L is -(CR⁸R⁹)(CR¹⁰R¹¹)-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R1, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R⁷ is independently oxo, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-6;
R⁸, R⁹, R¹⁰, and R¹¹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹² is hydrogen, optionally substituted C₁-C₆ alkyl, optionally substituted C₂-C₆ alkenyl, optionally substituted C₂-C₆ alkynyl, optionally substituted cycloalkyl, or optionally substituted heterocycloalkyl;
R¹³ is -OR¹⁴, NR¹⁵R¹⁶, or optionally substituted cycloalkyl;
R¹⁴ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R¹⁵ is optionally substituted cycloalkyl;
R¹⁶ is hydrogen, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl.

In some embodiments of a compound of Formula (XI), each R⁷ is independently oxo, deuterium, halogen, -CN, -OR^{b}, -NR^{c}R^{d}, or C₁-C₆ alkyl. In some embodiments of a compound of Formula (XI), each R⁷ is independently oxo, deuterium, halogen, or C₁-C₆ alkyl. In some embodiments of a compound of Formula (XI), each R⁷ is independently halogen or C₁-C₆ alkyl. In some embodiments of a compound of Formula (XI), each R⁷ is independently halogen.

In some embodiments of a compound of Formula (XI), n is 0-2. In some embodiments of a compound of Formula (XI), n is 0 or 1. In some embodiments of a compound of Formula (XI), n is 0. In some embodiments of a compound of Formula (XI), n is 1. In some embodiments of a compound of Formula (XI), n is 2.

In some embodiments of a compound of Formula (XI), R⁸, R⁹, R¹⁰, and R¹¹ are independently hydrogen, deuterium, halogen, -CN, -OR^{b}, -NR^{c}R^{d}, or optionally substituted C₁-C₆ alkyl. In some embodiments of a compound of Formula (XI), R⁸, R⁹, R¹⁰, and R¹¹ are independently hydrogen, deuterium, halogen, -CN, -OR^{b}, -NR^{c}R^{d}, or C₁-C₆ alkyl. In some embodiments of a compound of Formula (XI), R⁸, R⁹, R¹⁰, and R¹¹ are independently hydrogen, deuterium, halogen, or C₁-C₆ alkyl.. In some embodiments of a compound of Formula (XI), R⁸, R⁹, R¹⁰, and R¹¹ are independently hydrogen, halogen, or C₁-C₆ alkyl. In some embodiments of a compound of Formula (XI), R⁸, R⁹, R¹⁰, and R¹¹ are hydrogen.

In some embodiments of a compound of Formula (XI), R¹² is hydrogen, C₁-C₆ alkyl, cycloalkyl, or benzyl.

In some embodiments of a compound of Formula (XI), R¹² is hydrogen, C₁-C₆ alkyl, or cycloalkyl. In some embodiments of a compound of Formula (XI), R¹² is hydrogen or C₁-C₆ alkyl. In some embodiments of a compound of Formula (XI), R¹² is hydrogen.

In some embodiments of a compound of Formula (XI), R¹³ is NR¹⁵R¹⁶ or optionally substituted cycloalkyl. In some embodiments of a compound of Formula (XI), R¹³ is NR¹⁵R¹⁶ or cycloalkyl.

In some embodiments of a compound of Formula (XI), R¹⁵ is cycloalkyl. In some embodiments of a compound of Formula (XI), R¹⁵ is cyclopropyl, cyclobutyl, cyclopentyl, or cyclohexyl. In some embodiments of a compound of Formula (XI), R¹⁵ is cyclopropyl.

In some embodiments of a compound of Formula (XI), R¹⁶ is hydrogen or C₁-C₆ alkyl. In some embodiments of a compound of Formula (XI), R¹⁶ is hydrogen.

In some embodiments of a compound of Formula (XI), R¹³ is cycloalkyl. In some embodiments of a compound of Formula (XI), R¹³ is cyclopropyl, cyclobutyl, cyclopentyl, or cyclohexyl. In some embodiments of a compound of Formula (XI), R¹³ is cyclopropyl.

In some embodiments of a compound of Formula (XI),

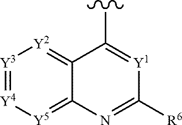

is

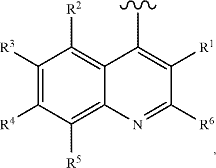

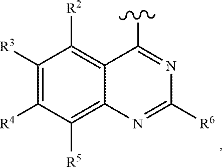

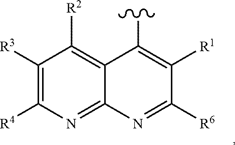

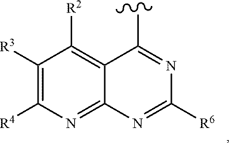

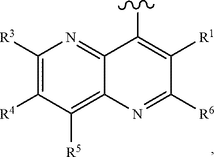

or

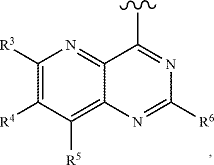

In some embodiments of a compound of Formula (XI),

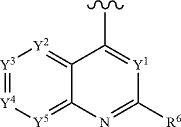

is

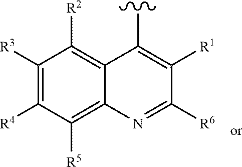

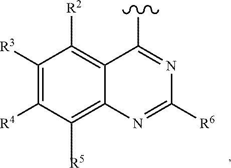

In some embodiments of a compound of Formula (XI),

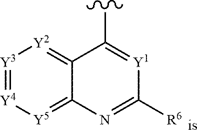

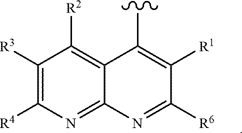

In some embodiments of a compound of Formula (XI), R¹ is hydrogen, deuterium, halogen, -CN, -OR^{b}, -NR^{c}R^{d}, or C₁-C₆ alkyl. In some embodiments of a compound of Formula (XI), R¹ is hydrogen, halogen, or X. In some embodiments of a compound of Formula (XI), R¹ is -CN. In some embodiments of a compound of Formula (XI), R¹ is halogen -CN. In some embodiments of a compound of Formula (XI), R¹ is hydrogen.

In some embodiments of a compound of Formula (XI), R² is hydrogen, deuterium, halogen, -CN, -OR^{b}, -NR^{c}R^{d}, or C₁-C₆ alkyl. In some embodiments of a compound of Formula (XI), R² is hydrogen, deuterium, halogen, or C₁-C₆ alkyl. In some embodiments of a compound of Formula (XI), R² is hydrogen or halogen. In some embodiments of a compound of Formula (XI), R² is hydrogen.

In some embodiments of a compound of Formula (XI), R³ is hydrogen, deuterium, halogen, -CN, -OR^{b}, -NR^{c}R^{d}, or C₁-C₆ alkyl. In some embodiments of a compound of Formula (XI), R³ is hydrogen, deuterium, halogen, -OR^{b}, or C₁-C₆ alkyl. In some embodiments of a compound of Formula (XI), R³ is hydrogen, -OR^{b}, or halogen. In some embodiments of a compound of Formula (XI), R³ is hydrogen or -OR^{b}. In some embodiments of a compound of Formula (XI), R³ is hydrogen. In some embodiments of a compound of Formula (XI), R³ is -OR^{b}.

In some embodiments of a compound of Formula (XI), R⁴ is hydrogen, deuterium, halogen, -CN, -OR^{b}, -NR^{c}R^{d}, or C₁-C₆ alkyl. In some embodiments of a compound of Formula (XI), R⁴ is hydrogen, deuterium, halogen, -OR^{b}, or C₁-C₆ alkyl. In some embodiments of a compound of Formula (XI), R⁴ is hydrogen or -OR^{b}. In some embodiments of a compound of Formula (XI), R⁴ is -OR^{b}. In some embodiments of a compound of Formula (XI), R⁴ is hydrogen.

In some embodiments of a compound of Formula (XI), R³ is OMe and R⁴ is OMe. In some embodiments of a compound of Formula (XI), R³ is OMe and R⁴ is hydrogen. In some embodiments of a compound of Formula (XI), R³ is hydrogen and R⁴ is OMe. In some embodiments of a compound of Formula (XI), R³ is hydrogen and R⁴ is OCD₃.

In some embodiments of a compound of Formula (XI), R⁵ is hydrogen, deuterium, halogen, -CN, -OR^{b}, -NR^{c}R^{d}, or C₁-C₆ alkyl. In some embodiments of a compound of Formula (XI), R⁵ is hydrogen, deuterium, halogen, or C₁-C₆ alkyl. In some embodiments of a compound of Formula (XI), R⁵ is hydrogen or halogen. In some embodiments of a compound of Formula (XI), R⁵ is hydrogen.

In some embodiments of a compound of Formula (XI), R⁶ is hydrogen, deuterium, halogen, -CN, -OR^{b}, -NR^{c}R^{d}, or C₁-C₆ alkyl. In some embodiments of a compound of Formula (XI), R⁶ is hydrogen, deuterium, halogen, or C₁-C₆ alkyl. In some embodiments of a compound of Formula (XI), R⁶ is hydrogen or halogen. In some embodiments of a compound of Formula (XI), R⁶ is hydrogen.

In some embodiments of a compound of Formula (VI)-(XI), each R^{a} is C₁-C₆ alkyl, cycloalkyl, heterocycloalkyl, aryl, or heteroaryl; each optionally substituted with deuterium, halogen, -OH, -OMe, or -NH₂. In some embodiments of a compound of Formula (VI)-(XI), each R^{a} is C₁-C₆ alkyl, cycloalkyl, or heterocycloalkyl; each optionally substituted with deuterium, halogen, -OH, -OMe, or -NH₂. In some embodiments of a compound of Formula (VI)-(XI), each R^{a} is C₁-C₆ alkyl or cycloalkyl; each optionally substituted with deuterium, halogen, -OH, -OMe, or -NH₂. In some embodiments of a compound of Formula (VI)-(XI), each R^{a} is C₁-C₆ alkyl optionally substituted with deuterium, halogen, -OH, -OMe, or -NH₂. In some embodiments of a compound of Formula (VI)-(XI), each R^{a} is C₁-C₆ alkyl or haloalkyl.

In some embodiments of a compound of Formula (VI)-(XI), each R^{b} is hydrogen, C₁-C₆ alkyl, cycloalkyl, heterocycloalkyl, aryl, or heteroaryl; each optionally substituted with deuterium, halogen, -OH, -OMe, or -NH₂. In some embodiments of a compound of Formula (VI)-(XI), each R^{b} is hydrogen, C₁-C₆ alkyl, cycloalkyl, or heterocycloalkyl; each optionally substituted with deuterium, halogen, -OH, -OMe, or -NH₂. In some embodiments of a compound of Formula (VI)-(XI), each R^{b} is hydrogen, C₁-C₆ alkyl or cycloalkyl; each optionally substituted with deuterium, halogen, -OH, -OMe, or -NH₂. In some embodiments of a compound of Formula (VI)-(XI), each R^{b} is hydrogen or C₁-C₆ alkyl optionally substituted with deuterium, halogen, -OH, -OMe, or -NH₂. In some embodiments of a compound of Formula (VI)-(XI), each R^{b} is hydrogen, C₁-C₆ alkyl, or haloalkyl.

In some embodiments of a compound of Formula (VI)-(XI), each R^{c} and R^{d} are each independently hydrogen, C₁-C₆ alkyl, cycloalkyl, heterocycloalkyl, aryl, or heteroaryl; each optionally substituted with deuterium, halogen, -OH, -OMe, or -NH₂. In some embodiments of a compound of Formula (VI)-(XI), each R^{c} and R^{d} are each independently hydrogen, C₁-C₆ alkyl, cycloalkyl, or heterocycloalkyl; each optionally substituted with deuterium, halogen, -OH, -OMe, or -NH₂. In some embodiments of a compound of Formula (VI)-(XI), each R^{c} and R^{d} are each independently hydrogen, C₁-C₆ alkyl or cycloalkyl; each optionally substituted with deuterium, halogen, -OH, -OMe, or -NH₂. In some embodiments of a compound of Formula (VI)-(XI), each R^{c} and R^{d} are each independently hydrogen or C₁-C₆ alkyl optionally substituted with deuterium, halogen, -OH, -OMe, or -NH₂. In some embodiments of a compound of Formula (VI)-(XI), each R^{c} and R^{d} are each independently hydrogen, C₁-C₆ alkyl, or haloalkyl.

In some embodiments of a compound of Formula (VI)-(XI), R^{c} and R^{d} are taken together with the nitrogen atom to which they are attached to form an optionally substituted heterocycloalkyl. In some embodiments of a compound of Formula (VI)-(XI), R^{c} and R^{d} are taken together with the nitrogen atom to which they are attached to form a heterocycloalkyl.

In some embodiments, the compound disclosed herein is selected from Table 1:

TABLE 1

| Ex | Name | Structure |
|---|---|---|
| 1 | N-(2-( 1-(2-amino-6,7-dimethoxyquinazolin-4-yl)piperidin-4-yl)ethyl)sulfamide | 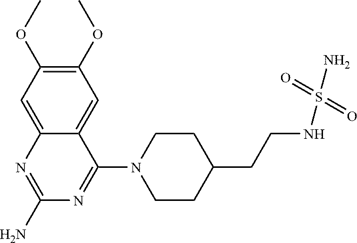 [SMILES: COc1cc2nc(N)nc(N3CCC(CCNS(N)(=O)=O)CC3)c2cc1OC] |
| 2 | N-(2-(1-(6,7-dimethoxy-2-(methylamino)quinazolin-4-yl)piperidin-4-yl)ethyl)sulfamide | 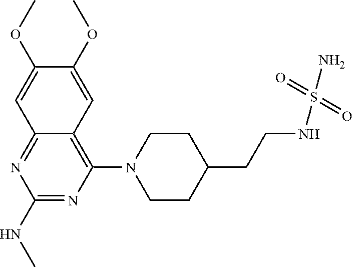 [SMILES: CNc1nc(N2CCC(CCNS(N)(=O)=O)CC2)c2cc(OC)c(OC)cc2n1] |
| 3 | N-(2-(1-(2-(dimethylamino)-6,7-dimethoxyquinazolin-4-yl)piperidin-4-yl)ethyl)sulfamide | 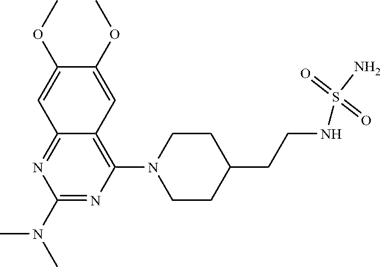 [SMILES: COc1cc2nc(N(C)C)nc(N3CCC(CCNS(N)(=O)=O)CC3)c2cc1OC] |
| 4 | N-(2-(1-(6,7-dimethoxy-2-(pyridin-2-yl)quinazolin-4-yl)piperidin-4-yl)ethyl)sulfamide | 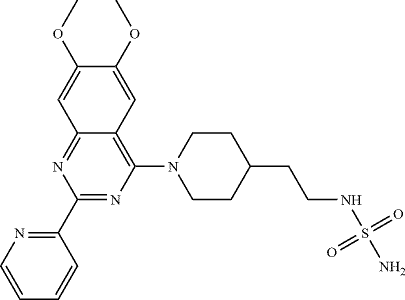 [SMILES: COc1cc2nc(-c3ccccn3)nc(N3CCC(CCNS(N)(=O)=O)CC3)c2cc1OC] |
| 5 | N-(2-(1-(6,7-dimethoxy-2-(pyridin-3 -yl)quinazolin-4-yl)piperidin-4-yl)ethyl)sulfamide | 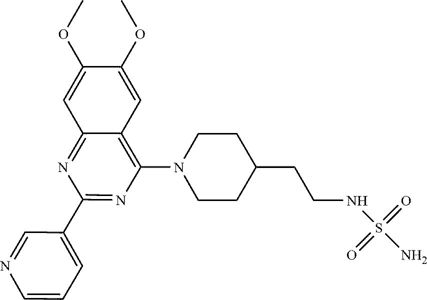 [SMILES: COc1cc2nc(-c3cccnc3)nc(N3CCC(CCNS(N)(=O)=O)CC3)c2cc1OC] |
| 6 | N-(2-(1-(6,7-dimethoxy-2-(pyridin-4-yl)quinazolin-4-yl)piperidin-4-yl)ethyl)sulfamide | 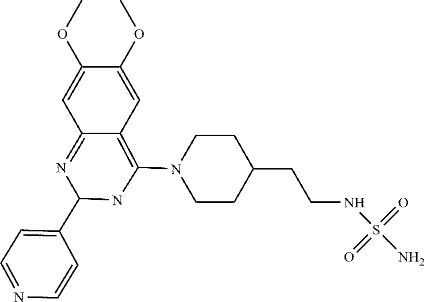 [SMILES: COc1cc2c(cc1OC)=C(N1CCC(CCNS(N)(=O)=O)CC1)NC(c1ccncc1)N=2] |
| 7 | N-(4-(4-(2-(sulfamoylamino)ethyl)piperidin-1-yl)pyrimidin-2-yl)nicotinamide | 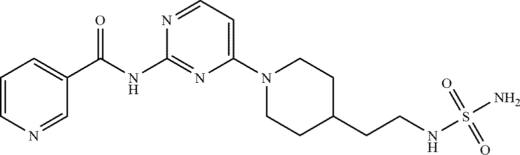 [SMILES: NS(=O)(=O)NCCC1CCN(c2ccnc(NC(=O)c3cccnc3)n2)CC1] |
| 8 | N-(2-(1-(6,7-dimethoxy-2-phenylquinazolin-4-yl)piperidin-4-yl)ethyl)sulfamide | 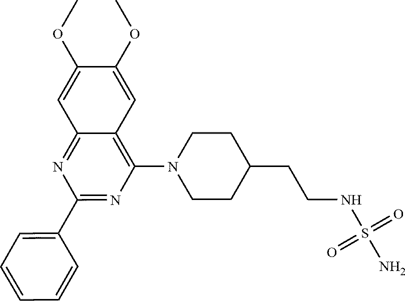 [SMILES: COc1cc2nc(-c3ccccc3)nc(N3CCC(CCNS(N)(=O)=O)CC3)c2cc1OC] |
| 9 | N-(2-(1-(6-(benzo[d]oxazol-6-yl)pyrimidin-4-yl)piperidin-4-yl)ethyl)sulfamide | 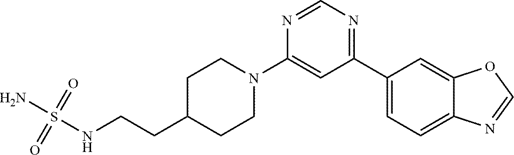 [SMILES: NS(=O)(=O)NCCC1CCN(c2cc(-c3ccc4ncoc4c3)ncn2)CC1] |
| 10 | N-(2-(1-(6-(benzo[d][1,3]dioxol-5-yl)pyrimidin-4-yl)piperidin-4-yl)ethyl)sulfamide | 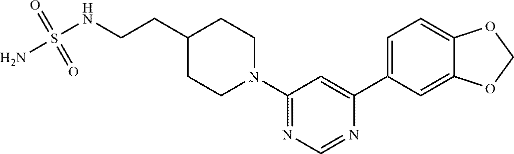 [SMILES: NS(=O)(=O)NCCC1CCN(c2cc(-c3ccc4c(c3)OCO4)ncn2)CC1] |
| 11 | N-(2-(1-(2-(pyridin-2-ylamino)pyrimidin-4-yl)piperidin-4-yl)ethyl)sulfamide | 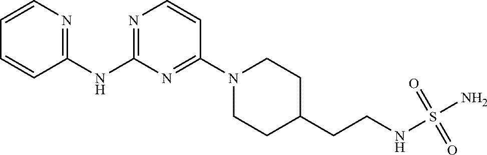 [SMILES: NS(=O)(=O)NCCC1CCN(c2ccnc(Nc3ccccn3)n2)CC1] |
| 12 | N-(2-(1-(2-(pyridin-3-ylamino)pyrimidin-4-yl)piperidin-4-yl)ethyl)sulfamide | 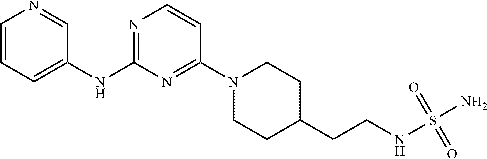 [SMILES: NS(=O)(=O)NCCC1CCN(c2ccnc(Nc3cccnc3)n2)CC1] |
| 13 | N-(2-(1-(2-(pyridin-4-ylamino)pyrimidin-4-yl)piperidin-4-yl)ethyl)sulfamide | 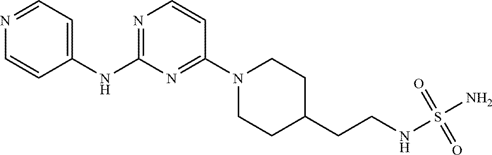 [SMILES: NS(=O)(=O)NCCC1CCN(c2ccnc(Nc3ccncc3)n2)CC1] |
| 14 | N-(2-(1-(2-(pyridin-3-yl)pyrimidin-4-yl)piperidin-4-yl)ethyl)sulfamide | 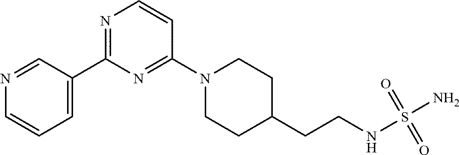 [SMILES: NS(=O)(=O)NCCC1CCN(c2ccnc(-c3cccnc3)n2)CC1] |
| 15 | N-(2-(1-(5,6-dimethylpyrimidin-4-yl)piperidin-4-yl)ethyl)sulfamide | 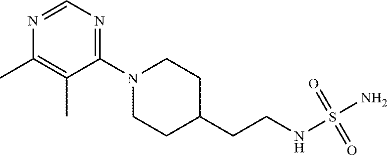 [SMILES: Cc1ncnc(N2CCC(CCNS(N)(=O)=O)CC2)c1C] |
| 16 | N-(2-( 1 -(6-methylpyrimidin-4-yl)piperidin-4-yl)ethyl)sulfamide | 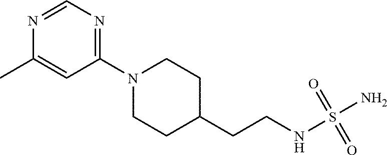 [SMILES: Cc1cc(N2CCC(CCNS(N)(=O)=O)CC2)ncn1] |
| 17 | N-(2-(1-(5-methylpyrimidin-4-yl)piperidin-4-yl)ethyl)sulfamide | 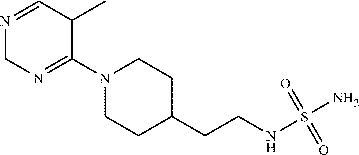 [SMILES: CC1C=NCN=C1N1CCC(CCNS(N)(=O)=O)CC1] |
| 18 | N-(2-( 1 -(pyrimidin-4-yl)piperidin-4-yl)ethyl)sulfamide | 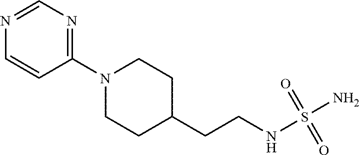 [SMILES: NS(=O)(=O)NCCC1CCN(c2ccncn2)CC1] |
| 19 | 6,7-dimethoxy-4-(4-(2-(sulfamoylamino)ethyl)piperidin-1 -yl)quinoline-3 -carboxamide | 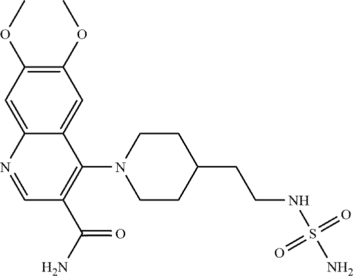 [SMILES: COc1cc2ncc(C(N)=O)c(N3CCC(CCNS(N)(=O)=O)CC3)c2cc1OC] |
| 20 | N-(2-(1-(3-cyano-6,7-dimethoxyquinolin-4-yl)piperidin-4-yl)ethyl)sulfamide | 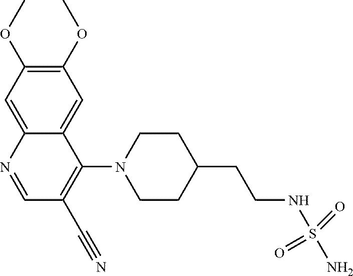 [SMILES: COc1cc2ncc(C#N)c(N3CCC(CCNS(N)(=O)=O)CC3)c2cc1OC] |
| 21 | N-(2-(1-(6,7-dimethoxyisoquinolin-1-yl)piperidin-4-yl)ethyl)sulfamide | 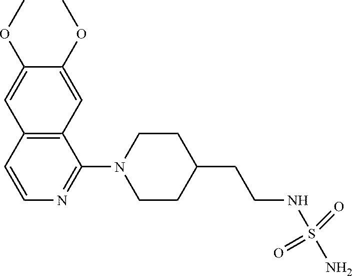 [SMILES: COc1cc2ccnc(N3CCC(CCNS(N)(=O)=O)CC3)c2cc1OC] |
| 22 | N-(2-( 1-(7-methyl-7H-purin-6-yl)piperidin-4-yl)ethyl)sulfamide | 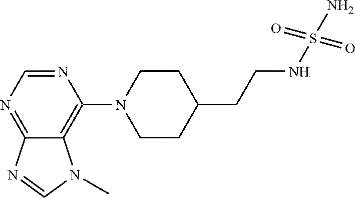 [SMILES: Cn1cnc2ncnc(N3CCC(CCNS(N)(=O)=O)CC3)c21] |
| 23 | N-(2-(1-(7H-purin-6-yl)piperidin-4-yl)ethyl)sulfamide | 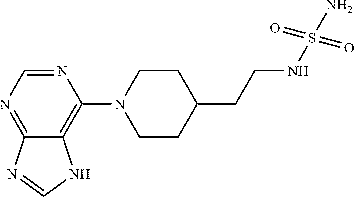 [SMILES: NS(=O)(=O)NCCC1CCN(c2ncnc3nc[nH]c23)CC1] |
| 24 | N-(2-( 1-(9-methyl-9H-purin-6-yl)piperidin-4-yl)ethyl)sulfamide | 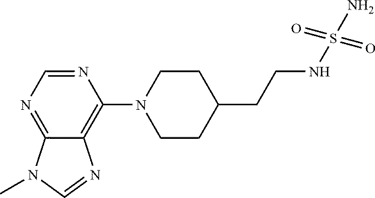 [SMILES: Cn1cnc2c(N3CCC(CCNS(N)(=O)=O)CC3)ncnc21] |
| 25 | N -(2-( 1-( 1-methyl-1H-pyrazolo [3,4-d]pyrimidin-4-yl)piperidin-4-yl)ethyl)sulfamide | 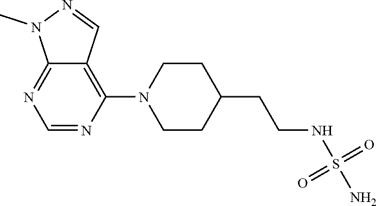 [SMILES: Cn1ncc2c(N3CCC(CCNS(N)(=O)=O)CC3)ncnc21] |
| 26 | N-(2-(1-( 1-phenyl-1H-pyrazolo [3,4-d]pyrimidin-4-yl)piperidin-4-yl)ethyl)sulfamide | 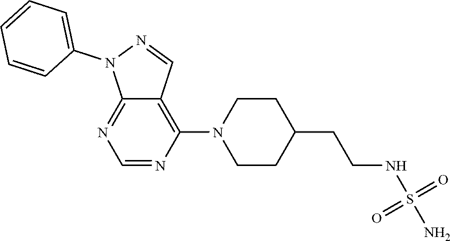 [SMILES: NS(=O)(=O)NCCC1CCN(c2ncnc3c2cnn3-c2ccccc2)CC1] |
| 27 | N-(2-(1-(2H-pyrazolo[4,3-d]pyrimidin-7 -yl)piperidin-4-yl)ethyl)sulfamide | 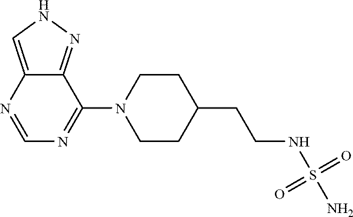 [SMILES: NS(=O)(=O)NCCC1CCN(c2ncnc3c[nH]nc23)CC1] |
| 28 | N-(2-(6,7-dimethoxyquinazolin-4-yl)-1,2,3,4-tetrahydroisoquinolin-5-yl)sulfamide | 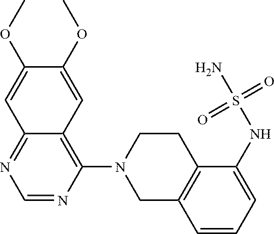 [SMILES: COc1cc2ncnc(N3CCc4c(cccc4NS(N)(=O)=O)C3)c2cc1OC] |
| 29 | 8-(6,7-dimethoxyquinazolin-4-yl)-2,8-diazaspiro[4.5]decane-2-sulfonamide | 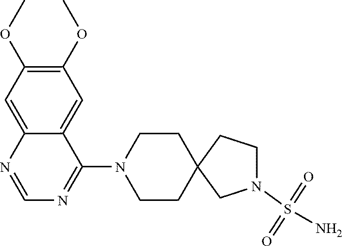 [SMILES: COc1cc2ncnc(N3CCC4(CC3)CCN(S(N)(=O)=O)C4)c2cc1OC] |
| 30 | 7-(6,7-dimethoxyquinazolin-4-yl)-2,7-diazaspiro[3.5]nonane-2-sulfonamide | 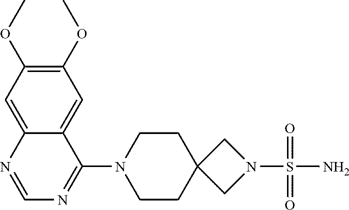 [SMILES: COc1cc2ncnc(N3CCC4(CC3)CN(S(N)(=O)=O)C4)c2cc1OC] |
| 31 | 2-(6,7-dimethoxyquinazolin-4-yl)-1,2,3,4-tetrahydroisoquinoline-5-sulfonamide | 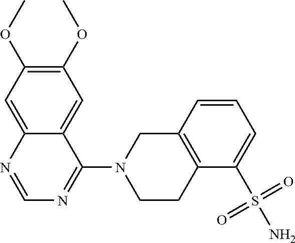 [SMILES: COc1cc2ncnc(N3CCc4c(cccc4S(N)(=O)=O)C3)c2cc1OC] |
| 32 | 7-(6,7-dimethoxyquinazolin-4-yl)-3,4-dihydroisoquinoline-2(1H)-sulfonamide | 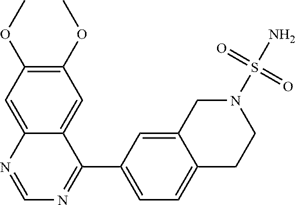 [SMILES: COc1cc2ncnc(-c3ccc4c(c3)CN(S(N)(=O)=O)CC4)c2cc1OC] |
| 33 | N-(3-(2-((6,7-dimethoxyquinazolin-4-yl)amino)ethyl)phenyl)sulfamide | 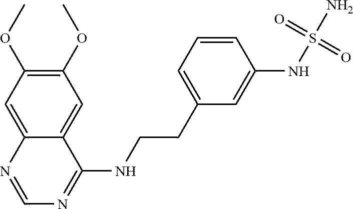 [SMILES: COc1cc2ncnc(NCCc3cccc(NS(N)(=O)=O)c3)c2cc1OC] |
| 34 | N-(4-(((6,7-dimethoxyquinazolin-4-yl)amino)methyl)phenyl)sulfamide | 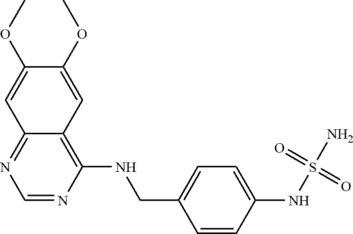 [SMILES: COc1cc2ncnc(NCc3ccc(NS(N)(=O)=O)cc3)c2cc1OC] |
| 35 | N-(2-( 1-(quinazolin-4-yl)piperidin-4-yl)ethyl)sulfamide | 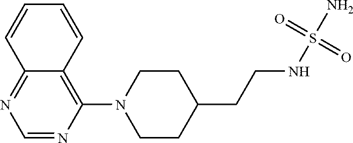 [SMILES: NS(=O)(=O)NCCC1CCN(c2ncnc3ccccc23)CC1] |
| 36 | N-(1-(6-(3,4-dimethoxyphenyl)pyrimidin-4-yl)piperidin-4-yl)sulfamide | 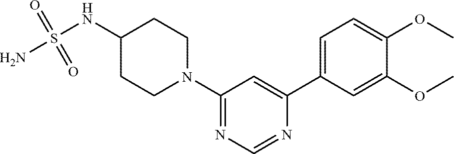 [SMILES: COc1ccc(-c2cc(N3CCC(NS(N)(=O)=O)CC3)ncn2)cc1OC] |
| 37 | N-((1-(6-(3,4-dimethoxyphenyl)pyrimidin-4-yl)piperidin-4-yl)methyl)sulfamide | 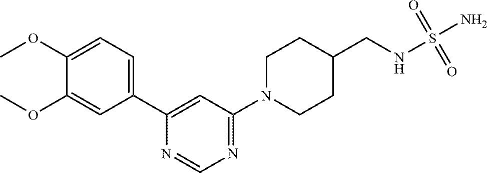 [SMILES: COc1ccc(-c2cc(N3CCC(CNS(N)(=O)=O)CC3)ncn2)cc1OC] |
| 38 | N-(2-(1-(6-(3,4-dimethoxyphenyl)pyrimidin-4-yl)piperidin-4-yl)ethyl)sulfamide | 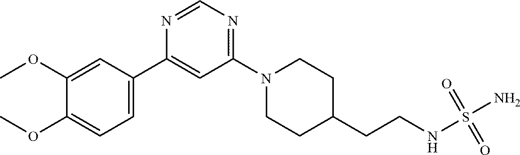 [SMILES: COc1ccc(-c2cc(N3CCC(CCNS(N)(=O)=O)CC3)ncn2)cc1OC] |
| 39 | N-(1 -(5,6-dimethylpyrimidin-4-yl)piperidin-4 -yl) sulfamide | 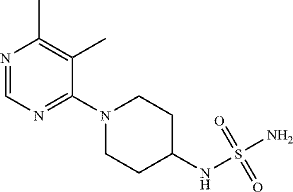 [SMILES: Cc1ncnc(N2CCC(NS(N)(=O)=O)CC2)c1C] |
| 40 | N-(2-(4-(6,7-dimethoxyquinazolin-4-yl)piperazin-1-yl)ethyl)sulfamide | 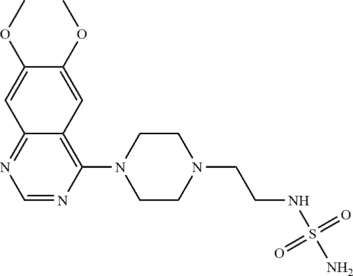 [SMILES: COc1cc2ncnc(N3CCN(CCNS(N)(=O)=O)CC3)c2cc1OC] |
| 41 | 6,7-dimethoxy-4-(5 -((4-methylpiperazin-1-yl)sulfonyl)-3,4-dihydroisoquinolin-2( 1H)-yl)quinazoline | 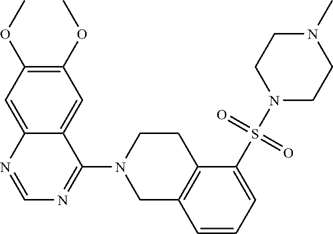 [SMILES: COc1cc2ncnc(N3CCc4c(cccc4S(=O)(=O)N4CCN(C)CC4)C3)c2cc1OC] |
| 42 | 2-(1-(6,7-dimethoxyquinazolin-4-yl)piperidin-4-yl)ethan-1-amino(N-ethyl-N-methyl)sulfonamide | 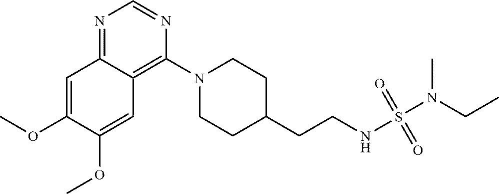 [SMILES: CCN(C)S(=O)(=O)NCCC1CCN(c2ncnc3cc(OC)c(OC)cc23)CC1] |
| 43 | N-(2-(1-(6,7-dimethoxyquinolin-4-yl)piperidin-4-yl)ethyl)sulfamide | 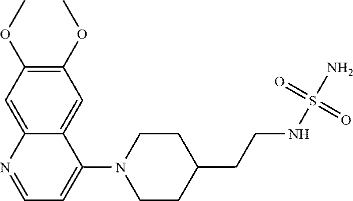 [SMILES: COc1cc2nccc(N3CCC(CCNS(N)(=O)=O)CC3)c2cc1OC] |
| 44 | N-(4,5-dimethoxy-2-(4-(2-(sulfamoylamino)ethyl)piperidine-1 -carbonyl)phenyl)acetamide | 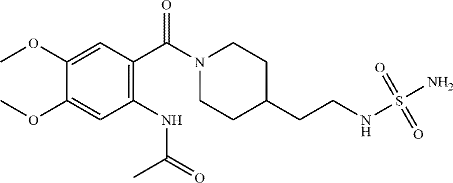 [SMILES: COc1cc(NC(C)=O)c(C(=O)N2CCC(CCNS(N)(=O)=O)CC2)cc1OC] |
| 45 | N-(2-(1-(2-(dimethylamino)-4,5-dimethoxybenzoyl)piperidin-4-yl)ethyl)sulfamide | 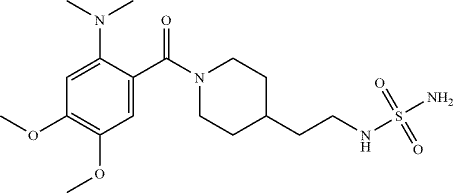 [SMILES: COc1cc(C(=O)N2CCC(CCNS(N)(=O)=O)CC2)c(N(C)C)cc1OC] |
| 46 | N-(2-(1-(2-amino-4,5-dimethoxybenzoyl)piperidin-4-yl)ethyl)sulfamide | 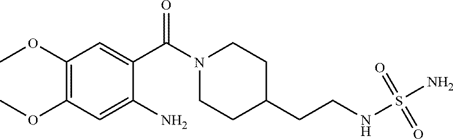 [SMILES: COc1cc(N)c(C(=O)N2CCC(CCNS(N)(=O)=O)CC2)cc1OC] |
| 47 | N-(2-(1-(3,4-dimethoxybenzoyl)piperidin-4-yl)ethyl)sulfamide | 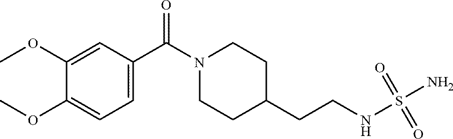 [SMILES: COc1ccc(C(=O)N2CCC(CCNS(N)(=O)=O)CC2)cc1OC] |
| 48 | N-(2-(1-(3-methoxybenzoyl)piperidin-4-yl)ethyl)sulfamide | 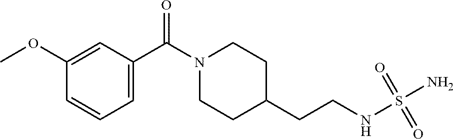 [SMILES: COc1cccc(C(=O)N2CCC(CCNS(N)(=O)=O)CC2)c1] |
| 49 | N-(2-(1-(4-methoxybenzoyl)piperidin-4-yl)ethyl)sulfamide | 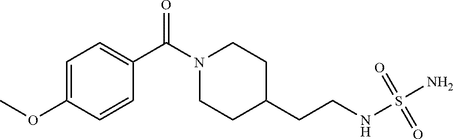 [SMILES: COc1ccc(C(=O)N2CCC(CCNS(N)(=O)=O)CC2)cc1] |
| 50 | N-(2-( 1-benzoylpiperidin-4-yl)ethyl)sulfamide | 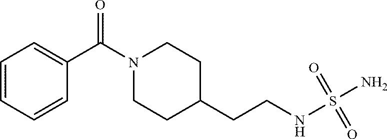 |
| 51 | N-(1-(6,7-dimethoxyquinazolin-4-yl)piperidin-4 -yl) sulfamide | 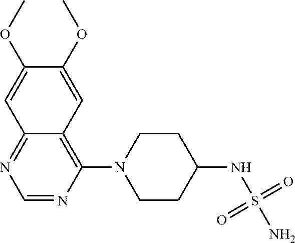 [SMILES: COc1cc2ncnc(N3CCC(NS(N)(=O)=O)CC3)c2cc1OC] |
| 52 | N-(2-(4-(6,7-dimethoxyquinazolin-4-yl)piperazin-1-yl)-2-oxoethyl)sulfamide | 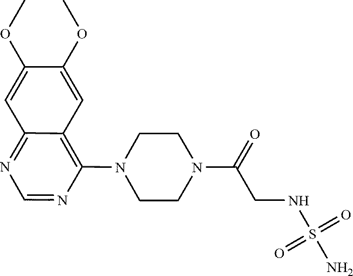 [SMILES: COc1cc2ncnc(N3CCN(C(=O)CNS(N)(=O)=O)CC3)c2cc1OC] |
| 53 | N-(4-((6,7-dimethoxyquinolin-4-yl)oxy)phenyl)cyclopropanesulfon amide | 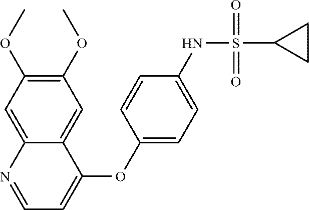 [SMILES: COc1cc2nccc(Oc3ccc(NS(=O)(=O)C4CC4)cc3)c2cc1OC] |
| 54 | N-(4-((7-methoxyquinolin-4-yl)oxy)phenyl)sulfamide | 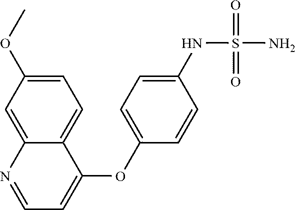 [SMILES: COc1ccc2c(Oc3ccc(NS(N)(=O)=O)cc3)ccnc2c1] |
| 55 | N-(4-(7-methoxyquinolin-4-yl)benzyl)sulfamide | 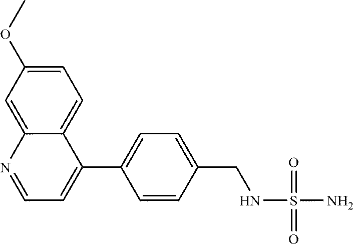 [SMILES: COc1ccc2c(-c3ccc(CNS(N)(=O)=O)cc3)ccnc2c1] |
| 56 | 4-(((6-fluoro-7-methoxyquinolin-4-yl)amino)methyl)benzene sulfonam ide | 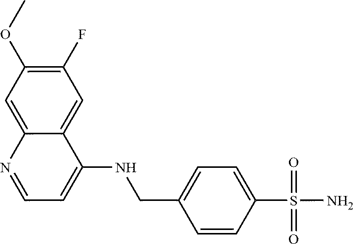 [SMILES: COc1cc2nccc(NCc3ccc(S(N)(=O)=O)cc3)c2cc1F] |
| 57 | 8-(3-cyano-6-fluoro-7-methoxyquinolin-4-yl)-2, 8-diazaspiro [4.5] decane -2 -sulfonamide | 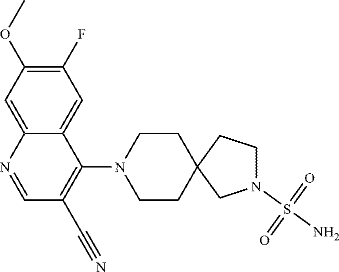 [SMILES: COc1cc2ncc(C#N)c(N3CCC4(CC3)CCN(S(N)(=O)=O)C4)c2cc1F] |
| 58 | 4-((7-methoxyquinolin-4-yl)oxy)benzenesulfonamide | 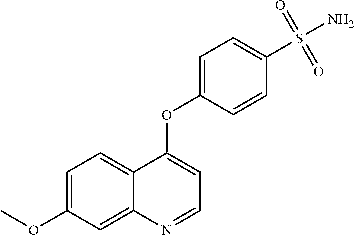 [SMILES: COc1ccc2c(Oc3ccc(S(N)(=O)=O)cc3)ccnc2c1] |
| 59 | N-(1-(7-methoxyquinolin-4-yl)indolin-5 -yl)sulfamide | 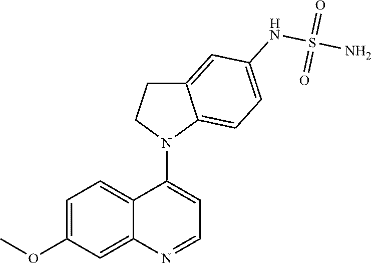 [SMILES: COc1ccc2c(N3CCc4cc(NS(N)(=O)=O)ccc43)ccnc2c1] |
| 60 | 7-(6,7-dimethoxy-3-methylquinolin-4-yl)-3,4-dihydroisoquinoline-2(1H)-sulfonamide | 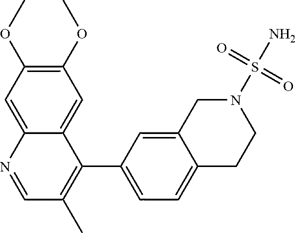 [SMILES: COc1cc2ncc(C)c(-c3ccc4c(c3)CN(S(N)(=O)=O)CC4)c2cc1OC] |
| 61 | N-(4-(6,7-dimethoxyquinolin-4-yl)benzyl)-N-methylsulfamide | 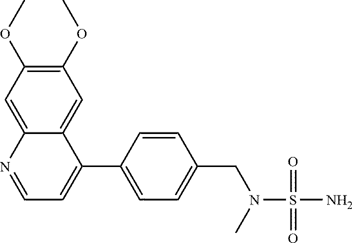 [SMILES: COc1cc2nccc(-c3ccc(CN(C)S(N)(=O)=O)cc3)c2cc1OC] |
| 62 | N-(2-fluoro-4-(7-methoxyquinolin-4-yl)benzyl)sulfamide | 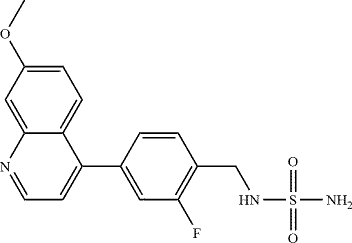 [SMILES: COc1ccc2c(-c3ccc(CNS(N)(=O)=O)c(F)c3)ccnc2c1] |
| 63 | N-(4-(7-methoxy-1,8-naphthyridin-4-yl)benzyl)sulfamide | 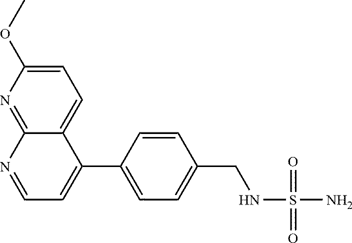 [SMILES: COc1ccc2c(-c3ccc(CNS(N)(=O)=O)cc3)ccnc2n1] |
| 64 | 8-(3-cyano-6,7-dimethoxyquinolin-4-yl)-2, 8-diazaspiro [4.5] decane -2 -sulfonamide | 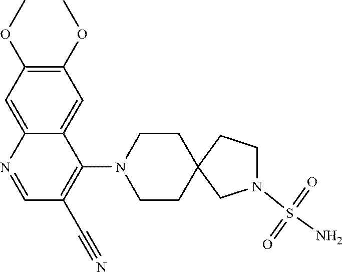 [SMILES: COc1cc2ncc(C#N)c(N3CCC4(CC3)CCN(S(N)(=O)=O)C4)c2cc1OC] |
| 65 | 8-(7-methoxyquinazolin-4-yl)-2,8-diazaspiro [4.5] decane -2 -sulfonamide | 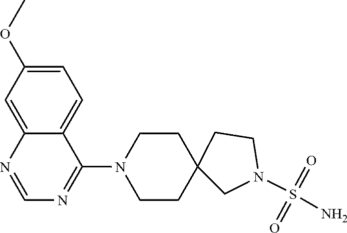 [SMILES: COc1ccc2c(N3CCC4(CC3)CCN(S(N)(=O)=O)C4)ncnc2c1] |
| 66 | N-(2-(1-(3-fluoro-6,7-dimethoxyquinolin-4-yl)piperidin-4-yl)ethyl)sulfamide | 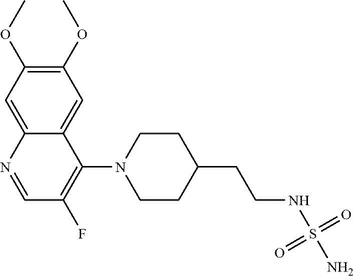 [SMILES: COc1cc2ncc(F)c(N3CCC(CCNS(N)(=O)=O)CC3)c2cc1OC] |
| 67 | N-(4-(3-fluoro-6,7-dimethoxyquinolin-4-yl)benzyl)sulfamide | 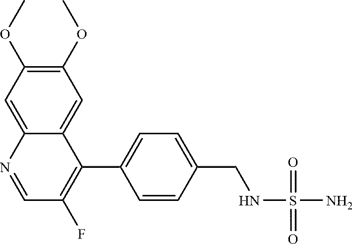 [SMILES: COc1cc2ncc(F)c(-c3ccc(CNS(N)(=O)=O)cc3)c2cc1OC] |
| 68 | N-cyclopropyl-N-(4-(6,7-dimethoxyquinolin-4-yl)benzyl)sulfamide | 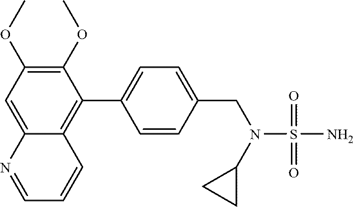 [SMILES: COc1cc2ncccc2c(-c2ccc(CN(C3CC3)S(N)(=O)=O)cc2)c1OC] |
| 69 | N-(4-(6,7-dimethoxyquinolin-4-yl) -2 -fluorobenzyl)sulfamide | 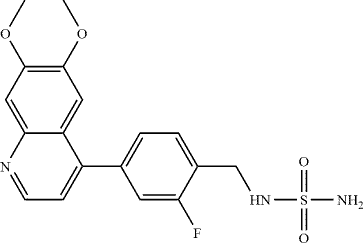 [SMILES: COc1cc2nccc(-c3ccc(CNS(N)(=O)=O)c(F)c3)c2cc1OC] |
| 70 | 8-(6-methoxyquinazolin-4-yl)-2,8-diazaspiro [4.5] decane -2 -sulfonamide | 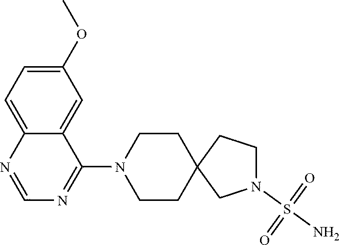 [SMILES: COc1ccc2ncnc(N3CCC4(CC3)CCN(S(N)(=O)=O)C4)c2c1] |
| 71 | N-(3-fluoro-4-((7-methoxyquinolin-4-yl)oxy)phenyl)sulfamide | 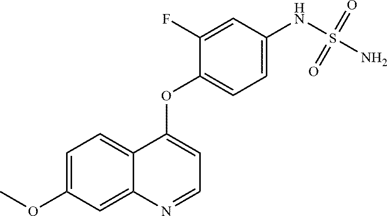 [SMILES: COc1ccc2c(Oc3ccc(NS(N)(=O)=O)cc3F)ccnc2c1] |
| 72 | N-(1 -(6-fluoro-7-methoxyquinolin-4-yl)indolin-5 -yl)sulfamide | 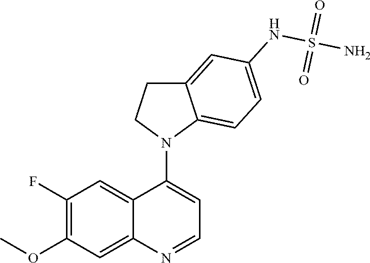 [SMILES: COc1cc2nccc(N3CCc4cc(NS(N)(=O)=O)ccc43)c2cc1F] |
| 73 | N-(4-((7-methoxy-1,8-naphthyridin-4-yl)oxy)phenyl)sulfamide | 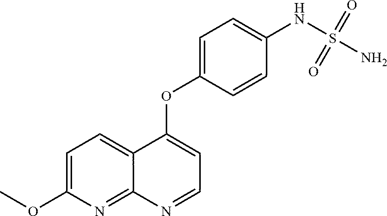 [SMILES: COc1ccc2c(Oc3ccc(NS(N)(=O)=O)cc3)ccnc2n1] |
| 74 | 4-((6-fluoro-7-methoxyquinolin-4-yl)oxy)benzenesulfonamide | 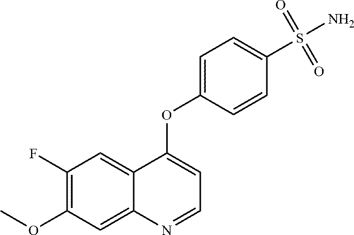 [SMILES: COc1cc2nccc(Oc3ccc(S(N)(=O)=O)cc3)c2cc1F] |
| 75 | N-(2-chloro-4-((7-methoxyquinolin-4-yl)oxy)phenyl)sulfamide | 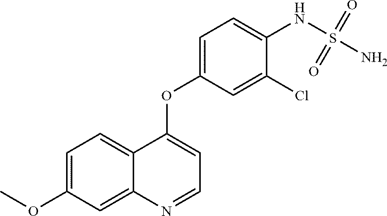 [SMILES: COc1ccc2c(Oc3ccc(NS(N)(=O)=O)c(Cl)c3)ccnc2c1] |
| 76 | 4-((3-cyano-6-fluoro-7-methoxyquinolin-4-yl)oxy)benzenesulfonamide | 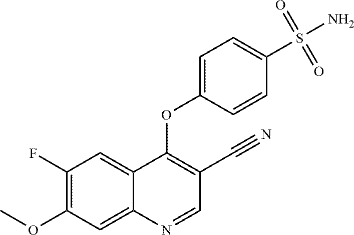 [SMILES: COc1cc2ncc(C#N)c(Oc3ccc(S(N)(=O)=O)cc3)c2cc1F] |
| 77 | N-(4-((7-methoxyquinolin-4-yl)oxy)phenyl)-N-methylsulfamide | 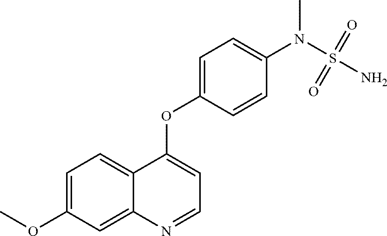 [SMILES: COc1ccc2c(Oc3ccc(N(C)S(N)(=O)=O)cc3)ccnc2c1] |
| 78 | N-(2-methoxy-4-((7-methoxyquinolin-4-yl)oxy)phenyl)sulfamide | 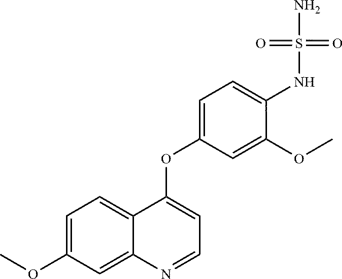 [SMILES: COc1ccc2c(Oc3ccc(NS(N)(=O)=O)c(OC)c3)ccnc2c1] |
| 79 | N-(2,5-difluoro-4-((7-methoxyquinolin-4-yl)oxy)phenyl)sulfamide | 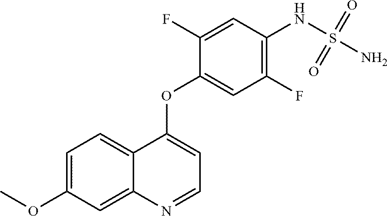 [SMILES: COc1ccc2c(Oc3cc(F)c(NS(N)(=O)=O)cc3F)ccnc2c1] |
| 80 | 4-((3-cyano-7-methoxyquinolin-4-yl)oxy)benzene sulfonamide | 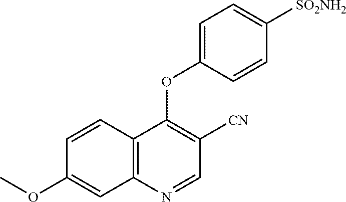 [SMILES: COc1ccc2c(Oc3ccc(S(N)(=O)=O)cc3)c(C#N)cnc2c1] |
| 81 | N-(4-((6,7-dimethoxyquinazolin-4-yl)oxy)benzyl)cyclopropanesulfon amide | 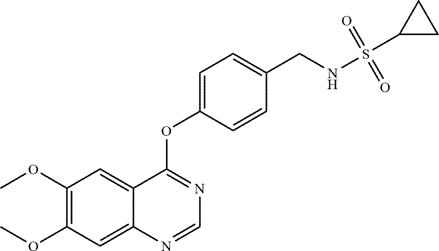 [SMILES: COc1cc2ncnc(Oc3ccc(CNS(=O)(=O)C4CC4)cc3)c2cc1OC] |
| 82 | N-(4-(1-((6,7-dimethoxyquinazolin-4-yl)amino)ethyl)phenyl)cyclopropa nesulfonamide | 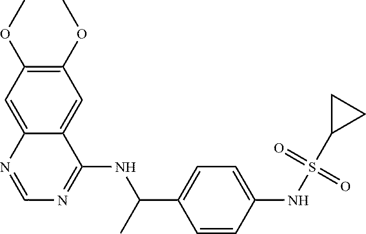 [SMILES: COc1cc2ncnc(NC(C)c3ccc(NS(=O)(=O)C4CC4)cc3)c2cc1OC] |
| 83 | N-(4-(3 -cyano-6-methoxy-1,7-naphthyridin-4-yl)benzyl)cyclopropanesulfonamid e | 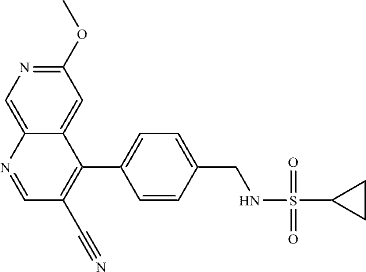 [SMILES: COc1cc2c(-c3ccc(CNS(=O)(=O)C4CC4)cc3)c(C#N)cnc2cn1] |
| 84 | N-(4-((6-fluoro-7-methoxyquinolin-4-yl)amino)phenyl)cyclopropane sulf onamide | 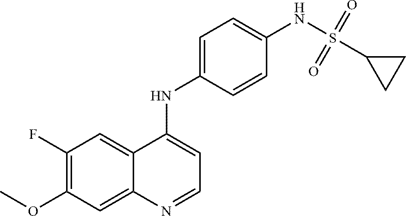 [SMILES: COc1cc2nccc(Nc3ccc(NS(=O)(=O)C4CC4)cc3)c2cc1F] |
| 85 | N-(4-(6,7-dimethoxyquinoxalin-2-yl)phenyl)cyclopropanesulfonamid e | 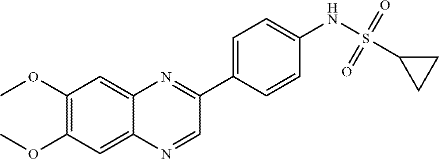 [SMILES: COc1cc2ncc(-c3ccc(NS(=O)(=O)C4CC4)cc3)nc2cc1OC] |
| 86 | N-(4-((6,7-dimethoxyquinolin-4-yl)oxy)phenyl)benzenesulfonamid e | 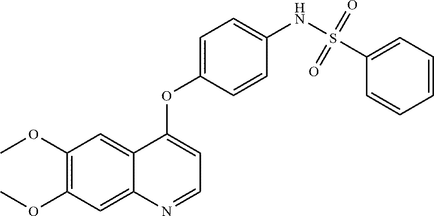 [SMILES: COc1cc2nccc(Oc3ccc(NS(=O)(=O)c4ccccc4)cc3)c2cc1OC] |
| 87 | N-(4-((6,7-dimethoxyquinolin-4-yl)amino)phenyl)cyclopropane sulf onamide | 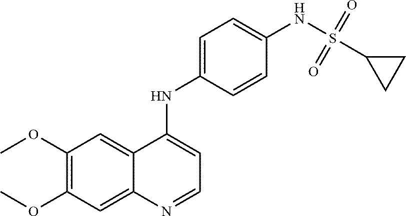 [SMILES: COc1cc2nccc(Nc3ccc(NS(=O)(=O)C4CC4)cc3)c2cc1OC] |
| 88 | N-(4-((3-cyano-6,7-dimethoxyquinolin-4-yl)amino)phenyl)cyclopropane sulf onamide | 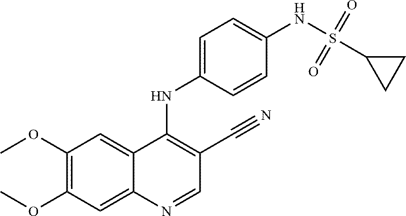 [SMILES: COc1cc2ncc(C#N)c(Nc3ccc(NS(=O)(=O)C4CC4)cc3)c2cc1OC] |
| 89 | N-(4-((6,7-dimethoxyquinolin-4-yl)oxy)-2,5-difluorophenyl)cyclopropanesulfon amide | 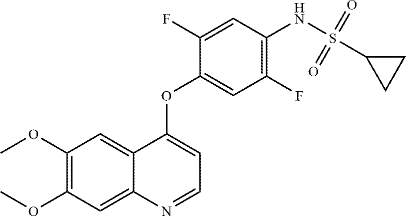 [SMILES: COc1cc2nccc(Oc3cc(F)c(NS(=O)(=O)C4CC4)cc3F)c2cc1OC] |
| 90 | N-(4-((7-(difluoromethoxy)quinolin-4-yl)oxy)phenyl)cyclopropanesulfon amide | 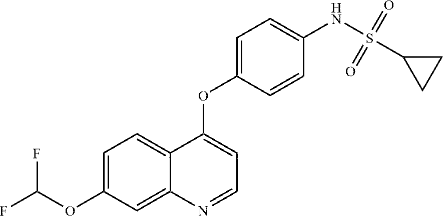 [SMILES: O=S(=O)(Nc1ccc(Oc2ccnc3cc(OC(F)F)ccc23)cc1)C1CC1] |
| 91 | N-(4-((6,7-dimethoxyquinolin-4-yl)amino)-2-fluorophenyl)cyclopropanesulfona mide | 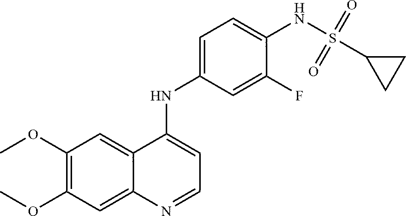 [SMILES: COc1cc2nccc(Nc3ccc(NS(=O)(=O)C4CC4)c(F)c3)c2cc1OC] |
| 92 | N-(4-(((6,7-dimethoxyquinolin-4-yl)oxy)methyl)phenyl)cyclopropan esulfonamide | 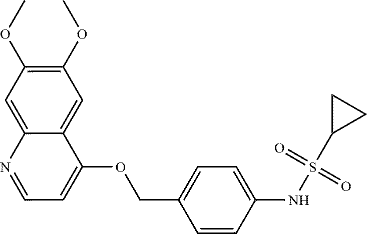 [SMILES: COc1cc2nccc(OCc3ccc(NS(=O)(=O)C4CC4)cc3)c2cc1OC] |
| 93 | N-(4-((6,7-dimethoxyquinazolin-4-yl)oxy)phenyl)cyclopropanesulfon amide | 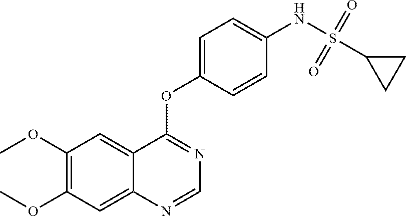 [SMILES: COc1cc2ncnc(Oc3ccc(NS(=O)(=O)C4CC4)cc3)c2cc1OC] |
| 94 | N-(4-((3-cyano-6,7-dimethoxyquinolin-4-yl)oxy)phenyl)cyclopropanesulfon amide | 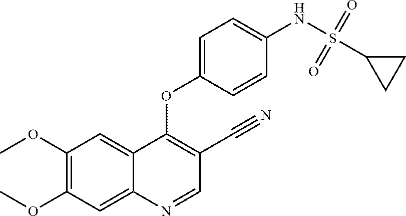 [SMILES: COc1cc2ncc(C#N)c(Oc3ccc(NS(=O)(=O)C4CC4)cc3)c2cc1OC] |
| 95 | N-(4-((3-fluoro-6,7-dimethoxyquinolin-4-yl)amino)phenyl)cyclopropane sulf onamide | 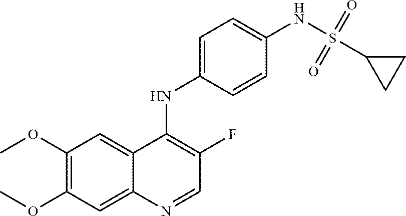 [SMILES: COc1cc2ncc(F)c(Nc3ccc(NS(=O)(=O)C4CC4)cc3)c2cc1OC] |
| 96 | N-(1-(6,7-dimethoxyquinolin-4-yl)indolin-5-yl)cyclopropanesulfonamide | 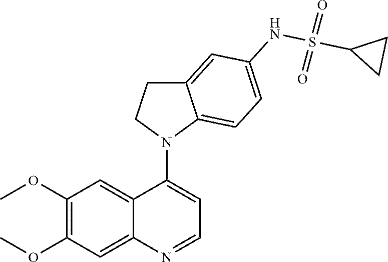 [SMILES: COc1cc2nccc(N3CCc4cc(NS(=O)(=O)C5CC5)ccc43)c2cc1OC] |
| 97 | 4-(2-(cyclopropylsulfonyl)- 1,2,3,4-tetrahydroisoquinolin-7-yl)-6,7-dimethoxyquinoline -3 -carbonitrile | 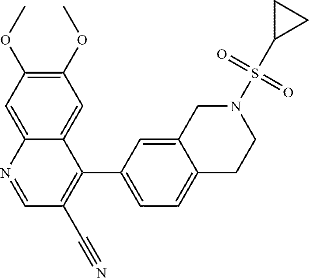 [SMILES: COc1cc2ncc(C#N)c(-c3ccc4c(c3)CN(S(=O)(=O)C3CC3)CC4)c2cc1OC] |
| 98 | 4-(2-(cyclopropylsulfonyl)-2,8-diazaspiro[4.5]decan-8-yl)-6,7-dimethoxyquinoline -3 -carbonitrile | 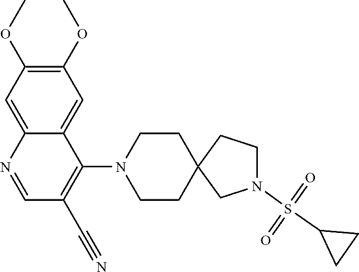 [SMILES: COc1cc2ncc(C#N)c(N3CCC4(CC3)CCN(S(=O)(=O)C3CC3)C4)c2cc1OC] |
| 99 | N-(4-((7-methoxyquinolin-4-yl)amino)phenyl)cyclopropane sulf onamide | 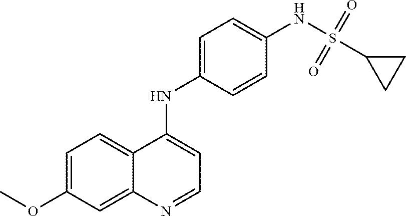 [SMILES: COc1ccc2c(Nc3ccc(NS(=O)(=O)C4CC4)cc3)ccnc2c1] |
| 100 | N-(4-((6,7-dimethoxyquinolin-4-yl)oxy)-3-fluorophenyl)cyclopropanesulfona mide | 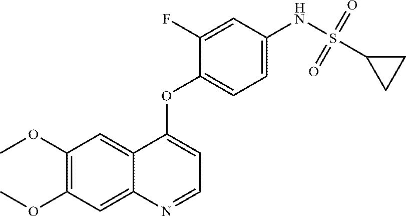 [SMILES: COc1cc2nccc(Oc3ccc(NS(=O)(=O)C4CC4)cc3F)c2cc1OC] |
| 101 | N-(4-((6,7-dimethoxyquinolin-4-yl)oxy)-2-fluorophenyl)cyclopropanesulfona mide | 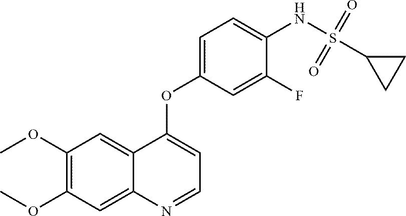 [SMILES: COc1cc2nccc(Oc3ccc(NS(=O)(=O)C4CC4)c(F)c3)c2cc1OC] |
| 102 | N-(4-((6-methoxy-1,5-naphthyridin-4-yl)oxy)phenyl)cyclopropanesulfon amide | 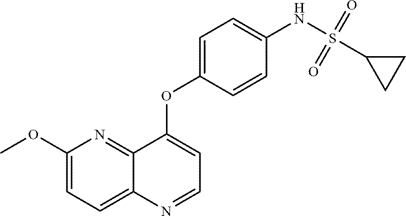 [SMILES: COc1ccc2nccc(Oc3ccc(NS(=O)(=O)C4CC4)cc3)c2n1] |
| 103 | N-(4-((6-methoxy-1,5-naphthyridin-4-yl)amino)phenyl)cyclopropane sulf onamide | 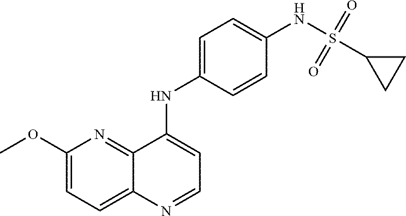 [SMILES: COc1ccc2nccc(Nc3ccc(NS(=O)(=O)C4CC4)cc3)c2n1] |
| 104 | N-(4-((7-methoxyquinolin-4-yl)oxy)phenyl)cyclopropanesulfon amide | 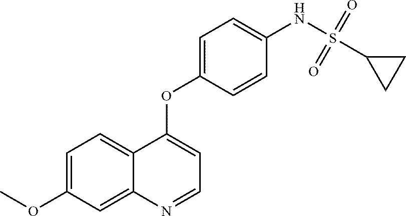 [SMILES: COc1ccc2c(Oc3ccc(NS(=O)(=O)C4CC4)cc3)ccnc2c1] |
| 105 | N-(4-(pyrido[2,3-d]pyrimidin-4-yloxy)phenyl)cyclopropanesulfona mide | 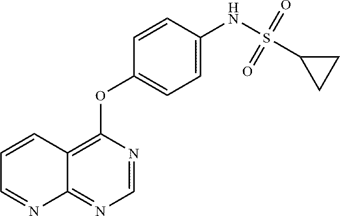 [SMILES: O=S(=O)(Nc1ccc(Oc2ncnc3ncccc23)cc1)C1CC1] |
| 106 | N-(4-(((6,7-dimethoxyquinolin-4-yl)amino)methyl)phenyl)cycloprop anesulfonamide | 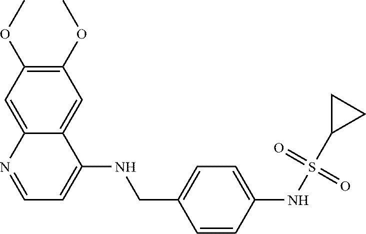 [SMILES: COc1cc2nccc(NCc3ccc(NS(=O)(=O)C4CC4)cc3)c2cc1OC] |
| 107 | N-(4-((6,7-dimethoxyquinolin-4-yl)oxy)phenyl)-1-methylcyclopropane -1 -sulfonamide | 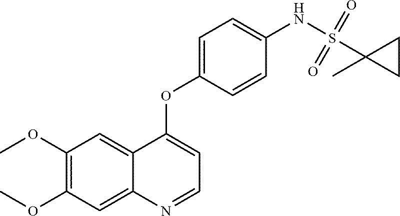 [SMILES: COc1cc2nccc(Oc3ccc(NS(=O)(=O)C4(C)CC4)cc3)c2cc1OC] |
| 108 | N-(2-(1-(6,7-dimethoxyquinazolin-4-yl)piperidin-4-yl)ethyl)cyclopropylsulfamide | 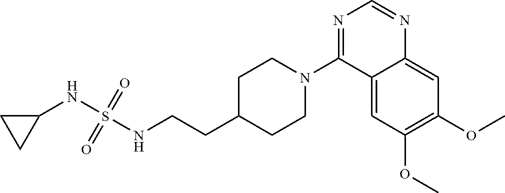 [SMILES: COc1cc2ncnc(N3CCC(CCNS(=O)(=O)NC4CC4)CC3)c2cc1OC] |
| 109 | 2-(cyclopropylsulfonyl)-8-(6,7-dimethoxyquinazolin-4-yl)-2,8-diazaspiro [4.5] decane | 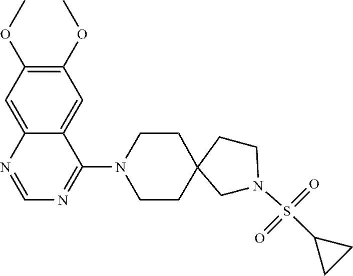 [SMILES: COc1cc2ncnc(N3CCC4(CC3)CCN(S(=O)(=O)C3CC3)C4)c2cc1OC] |
| 110 | N-(4-((6,7-dimethoxyquinolin-4-yl)oxy)-2,6-difluorophenyl)cyclopropanesulfon amide | 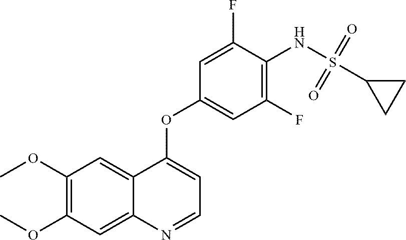 [SMILES: COc1cc2nccc(Oc3cc(F)c(NS(=O)(=O)C4CC4)c(F)c3)c2cc1OC] |
| 111 | N-(6′,7′-dimethoxy-3,4-dihydro-2H-[ 1,4′-biquinolin] -6-yl)cyclopropanesulfonamide | 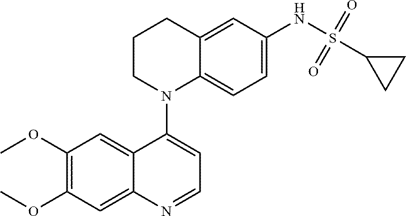 [SMILES: COc1cc2nccc(N3CCCc4cc(NS(=O)(=O)C5CC5)ccc43)c2cc1OC] |
| 112 | N-(4-((6-fluoro-7-methoxyquinolin-4-yl)oxy)phenyl)cyclopropanesulfon amide | 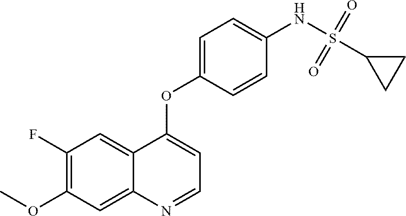 [SMILES: COc1cc2nccc(Oc3ccc(NS(=O)(=O)C4CC4)cc3)c2cc1F] |
| 113 | N-(4-(6,7-dimethoxyquinazolin-4-yl)phenyl)sulfamide | 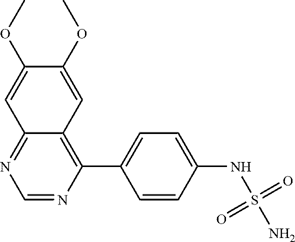 [SMILES: COc1cc2ncnc(-c3ccc(NS(N)(=O)=O)cc3)c2cc1OC] |
| 114 | N-(4-((6,7-dimethoxyquinazolin-4-yl)amino)phenyl)sulfamide | 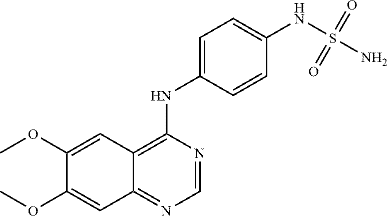 [SMILES: COc1cc2ncnc(Nc3ccc(NS(N)(=O)=O)cc3)c2cc1OC] |
| 115 | 2-(1-(6,7-dimethoxyquinazolin-4-yl)piperidin-4-yl)-N,N-dimethylethan-1-amine | 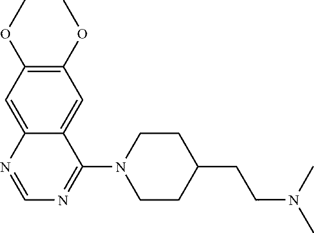 [SMILES: COc1cc2ncnc(N3CCC(CCN(C)C)CC3)c2cc1OC] |
| 116 | N-(2-(1-(6,7-dimethoxyquinazolin-4-yl)piperidin-4-yl)ethyl)benzenesulfonamide | 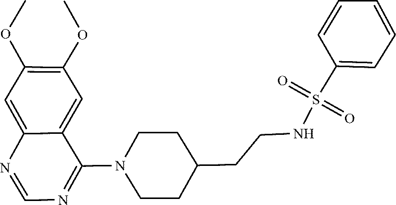 [SMILES: COc1cc2ncnc(N3CCC(CCNS(=O)(=O)c4ccccc4)CC3)c2cc1OC] |
| 117 | 1-(2-(1-(6,7-dimethoxyquinazolin-4-yl)piperidin-4-yl)ethyl)urea | 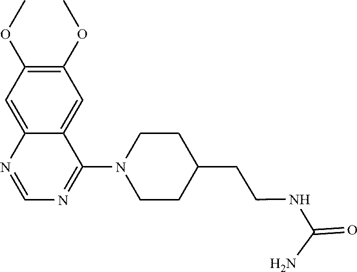 [SMILES: COc1cc2ncnc(N3CCC(CCNC(N)=O)CC3)c2cc1OC] |
| 118 | N-(2-(1-(2-(dimethylamino)-6,7-dimethoxyquinazolin-4-yl)piperidin-4-yl)ethyl)formamide | 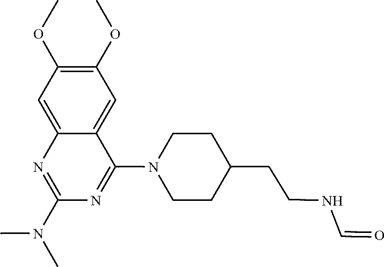 [SMILES: COc1cc2nc(N(C)C)nc(N3CCC(CCNC=O)CC3)c2cc1OC] |
| 119 | 2-amino-1-(4-(6,7-dimethoxyquinazolin-4-yl)piperazin-1 -yl)ethan-1 -one | 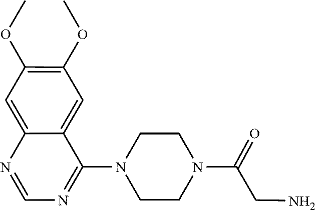 [SMILES: COc1cc2ncnc(N3CCN(C(=O)CN)CC3)c2cc1OC] |
| 120 | N-(4-(5,6-dimethoxy-1H-benzo[d]imidazol-1-yl)benzyl)sulfamide | 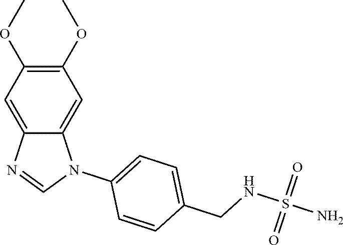 [SMILES: COc1cc2ncn(-c3ccc(CNS(N)(=O)=O)cc3)c2cc1OC] |
| 121 | 6-(6,7-dimethoxyquinazolin-4-yl)-3,4-dihydroisoquinoline-2(1H)-sulfonamide | 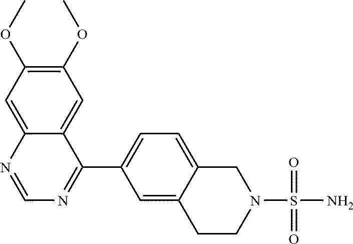 [SMILES: COc1cc2ncnc(-c3ccc4c(c3)CCN(S(N)(=O)=O)C4)c2cc1OC] |
| 122 | N-(5-(6,7-dimethoxyquinazolin-4-yl)-2,3-dihydro-1H-inden-2-yl)sulfamide | 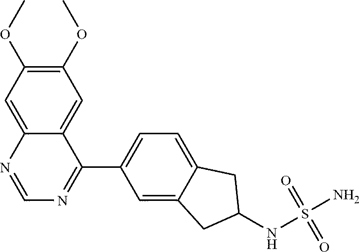 [SMILES: COc1cc2ncnc(-c3ccc4c(c3)CC(NS(N)(=O)=O)C4)c2cc1OC] |
| 123 | N-(2-(1-(4,5-dimethoxy-2-(methylamino)benzoyl)piperidin-4-yl)ethyl)sulfamide | 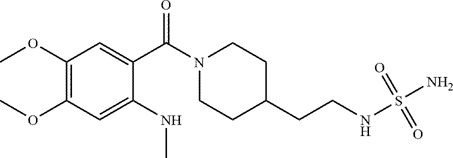 [SMILES: CNc1cc(OC)c(OC)cc1C(=O)N1CCC(CCNS(N)(=O)=O)CC1] |
| 124 | N-(2-(1-(4,5-dimethoxypicolinoyl)piperidin-4-yl)ethyl)sulfamide | 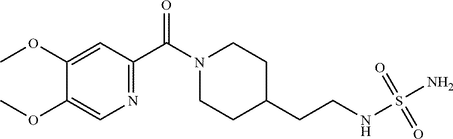 [SMILES: COc1cnc(C(=O)N2CCC(CCNS(N)(=O)=O)CC2)cc1OC] |
| 125 | N-(4-((6,7-dimethoxyquinolin-4-yl)oxy)phenyl)sulfamide | 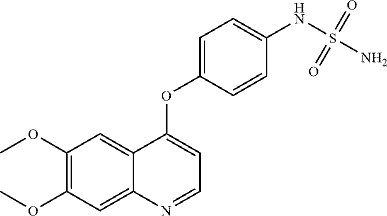 [SMILES: COc1cc2nccc(Oc3ccc(NS(N)(=O)=O)cc3)c2cc1OC] |
| 126 | N-(4-((6,7-dimethoxy-2-oxoquinolin-1(2H)-yl)methyl)phenyl) sulfamide | 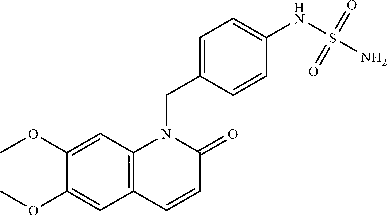 [SMILES: COc1cc2ccc(=O)n(Cc3ccc(NS(N)(=O)=O)cc3)c2cc1OC] |
| 127 | N-methyl-4-(4-(sulfamoylamino)phenoxy)picolina mide | 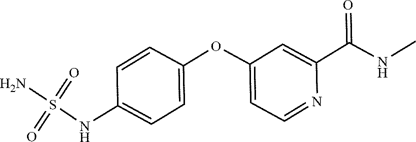 [SMILES: CNC(=O)c1cc(Oc2ccc(NS(N)(=O)=O)cc2)ccn1] |
| 128 | N-(4-(7-methyl-7H-purin-6-yl)benzyl)sulfamide | 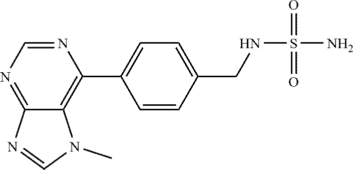 [SMILES: Cn1cnc2ncnc(-c3ccc(CNS(N)(=O)=O)cc3)c21] |
| 129 | N-(2-(1-(2-cyano-4,5-dimethoxybenzoyl)piperidin-4-yl)ethyl)sulfamide | 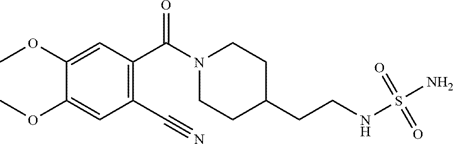 [SMILES: COc1cc(C#N)c(C(=O)N2CCC(CCNS(N)(=O)=O)CC2)cc1OC] |
| 130 | N-methyl-6-(4-(2-(sulfamoylamino)ethyl)piperidin-1-yl)pyrimidine-4-carboxamide | 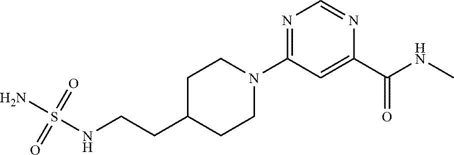 [SMILES: CNC(=O)c1cc(N2CCC(CCNS(N)(=O)=O)CC2)ncn1] |
| 131 | N-(6-(6,7-dimethoxyquinazolin-4-yl)-1,2,3,4-tetrahydronaphthalen-2-yl)sulfamide | 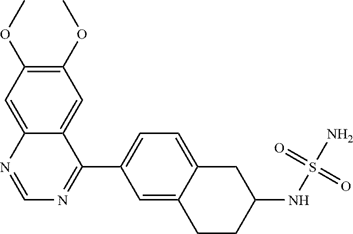 [SMILES: COc1cc2ncnc(-c3ccc4c(c3)CCC(NS(N)(=O)=O)C4)c2cc1OC] |
| 132 | N-(2-(1-(6,7-dimethoxyquinazolin-4-yl)piperidin-4-yl)ethyl)morpholine-4-sulfonamide | 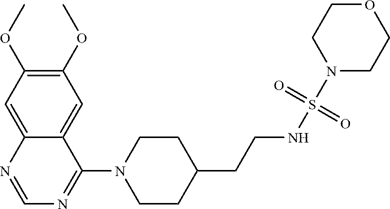 [SMILES: COc1cc2ncnc(N3CCC(CCNS(=O)(=O)N4CCOCC4)CC3)c2cc1OC] |
| 133 | N-(3,4-dimethoxyphenyl)-4-((sulfamoylamino)methyl)cyclohe xane-1-carboxamide | 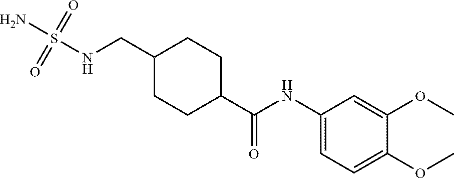 [SMILES: COc1ccc(NC(=O)C2CCC(CNS(N)(=O)=O)CC2)cc1OC] |
| 134 | N-(4-((6,7-dimethoxyquinolin-4-yl)amino)phenyl)sulfamide | 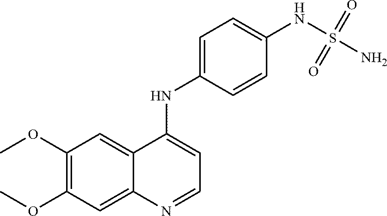 [SMILES: COc1cc2nccc(Nc3ccc(NS(N)(=O)=O)cc3)c2cc1OC] |
| 135 | N-(2-(1-(1-methyl-1H-pyrazolo[3,4-b]pyridin-4-yl)piperidin-4-yl)ethyl)sulfamide | 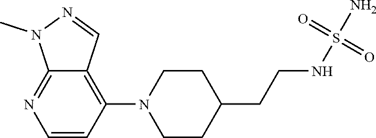 [SMILES: Cn1ncc2c(N3CCC(CCNS(N)(=O)=O)CC3)ccnc21] |
| 136 | 4-(3-cyano-6,7-dimethoxyquinolin-4-yl)benzenesulfonamide | 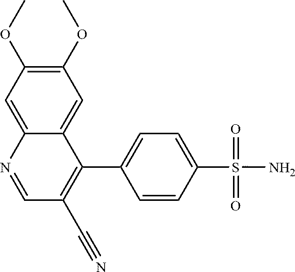 [SMILES: COc1cc2ncc(C#N)c(-c3ccc(S(N)(=O)=O)cc3)c2cc1OC] |
| 137 | N-(2-(6,7-dimethoxyquinazolin-4-yl)-1,2,3,4-tetrahydroisoquinolin-6-yl)sulfamide | 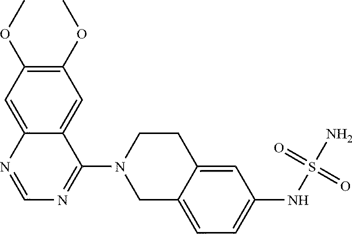 [SMILES: COc1cc2ncnc(N3CCc4cc(NS(N)(=O)=O)ccc4C3)c2cc1OC] |
| 138 | N-(2-(1-(7-bromo-5-methyl-SH-pyrrolo[3,2-d]pyrimidin-4-yl)piperidin-4-yl)ethyl)sulfamide | 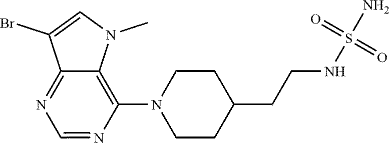 [SMILES: Cn1cc(Br)c2ncnc(N3CCC(CCNS(N)(=O)=O)CC3)c21] |
| 139 | 2-(1-(6,7-dimethoxyquinazolin-4-yl)piperidin-4-yl)ethan-1-amino(N-ethyl)sulfonamide | 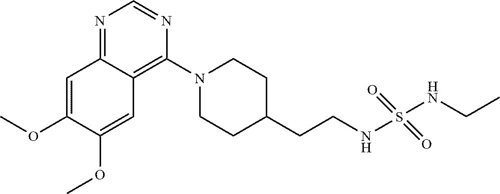 [SMILES: CCNS(=O)(=O)NCCC1CCN(c2ncnc3cc(OC)c(OC)cc23)CC1] |
| 140 | N-(4-(1-((6,7-dimethoxyquinazolin-4-yl)amino)ethyl)phenyl) sulfamide | 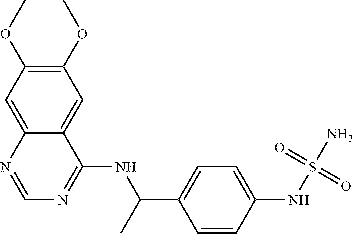 [SMILES: COc1cc2ncnc(NC(C)c3ccc(NS(N)(=O)=O)cc3)c2cc1OC] |
| 141 | N-(2-(1-(7-methyl-8-oxo-8,9-dihydro-7H-purin-6-yl)piperidin-4-yl)ethyl)sulfamide | 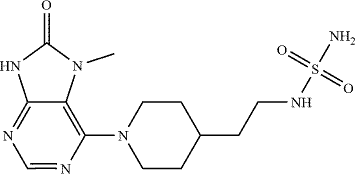 [SMILES: Cn1c(=O)[nH]c2ncnc(N3CCC(CCNS(N)(=O)=O)CC3)c21] |
| 142 | 2-(1-(6,7-dimethoxyquinazolin-4-yl)piperidin-4-yl)ethan-1-amino(N-prop-2-yl)sulfonamide | 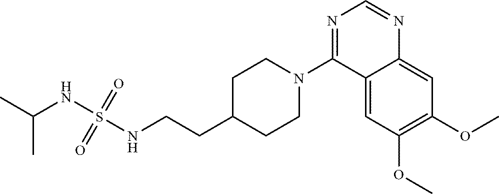 [SMILES: COc1cc2ncnc(N3CCC(CCNS(=O)(=O)NC(C)C)CC3)c2cc1OC] |
| 143 | 2-(1-(6,7-dimethoxyquinazolin-4-yl)piperidin-4-yl)ethan-1 -amino(N,N-dimethyl)sulfonamide | 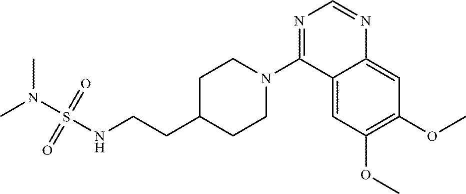 |
| 144 | N-(4-(6,7-dimethoxyquinazolin-4-yl)benzyl)sulfamide | 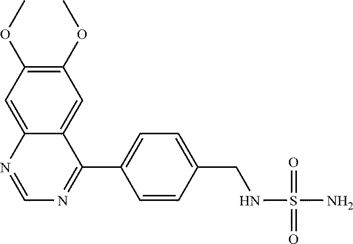 [SMILES: COc1cc2ncnc(-c3ccc(CNS(N)(=O)=O)cc3)c2cc1OC] |
| 145 | N-(4-(6,7-dimethoxyquinazolin-4-yl)phenethyl)sulfamide | 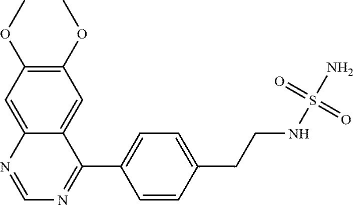 [SMILES: COc1cc2ncnc(-c3ccc(CCNS(N)(=O)=O)cc3)c2cc1OC] |
| 146 | N-(2-(1-(6,7-dimethoxyquinazolin-4-yl)piperidin-4-yl)ethyl)-1,1,1-trifluoromethanesulfonamide | 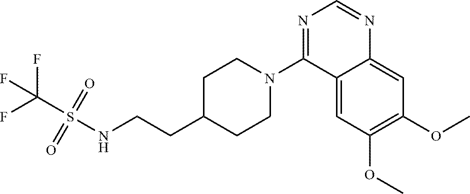 [SMILES: COc1cc2ncnc(N3CCC(CCNS(=O)(=O)C(F)(F)F)CC3)c2cc1OC] |
| 147 | N-(2-(1-(6,7-dimethoxyquinolin-4-yl)piperidin-4-yl)ethyl)morpholine-4-sulfonamide | 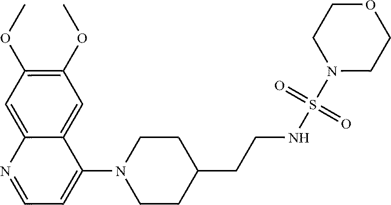 [SMILES: COc1cc2nccc(N3CCC(CCNS(=O)(=O)N4CCOCC4)CC3)c2cc1OC] |
| 148 | 7-(6,7-dimethoxyquinolin-4-yl)-3,4-dihydroisoquinoline-2(1H)-sulfonamide | 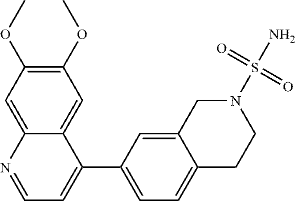 [SMILES: COc1cc2nccc(-c3ccc4c(c3)CN(S(N)(=O)=O)CC4)c2cc1OC] |
| 149 | 7-(3-cyano-6,7-dimethoxyquinolin-4-yl)-3,4-dihydroisoquinoline-2(1H)-sulfonamide | 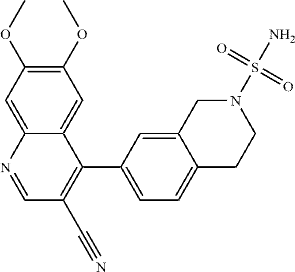 [SMILES: COc1cc2ncc(C#N)c(-c3ccc4c(c3)CN(S(N)(=O)=O)CC4)c2cc1OC] |
| 150 | 7-(quinazolin-4-yl)-3,4-dihydroisoquinoline-2(1H)-sulfonamide | 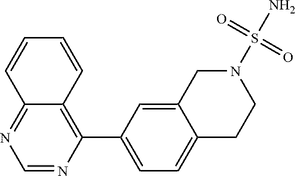 [SMILES: NS(=O)(=O)N1CCc2ccc(-c3ncnc4ccccc34)cc2C1] |
| 151 | N-(4-((6,7-dimethoxy-4-oxoquinolin-1 (4H)-yl)methyl)phenyl) sulfamide | 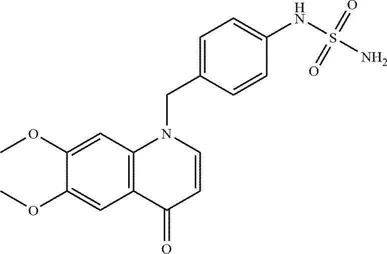 [SMILES: COc1cc2c(=O)ccn(Cc3ccc(NS(N)(=O)=O)cc3)c2cc1OC] |
| 152 | N-(2-(4-((3,4-dimethoxyphenyl)sulfonyl)piperazi n-1-yl)ethyl)sulfamide | 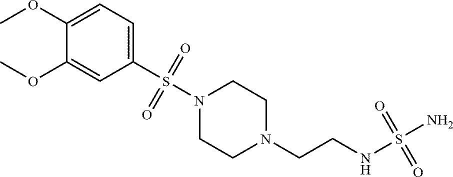 [SMILES: COc1ccc(S(=O)(=O)N2CCN(CCNS(N)(=O)=O)CC2)cc1OC] |
| 153 | N-(4-((6,7-dimethoxyquinolin-4-yl)amino)phenyl)benzenesulfonam ide | 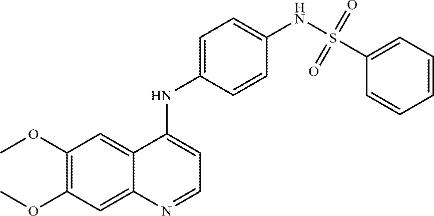 [SMILES: COc1cc2nccc(Nc3ccc(NS(=O)(=O)c4ccccc4)cc3)c2cc1OC] |
| 154 | N-(2-(1-(6,7-dimethoxyquinoline-4-carbonyl)piperidin-4-yl)ethyl)sulfamide | 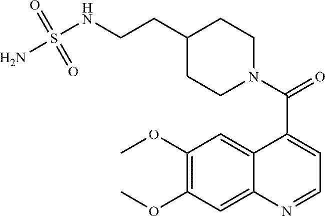 [SMILES: COc1cc2nccc(C(=O)N3CCC(CCNS(N)(=O)=O)CC3)c2cc1OC] |
| 155 | N-(2-(1-(6,7-dimethoxyquinazolin-4-yl)piperidin-4-yl)ethyl)pyridine-2-sulfonamide | 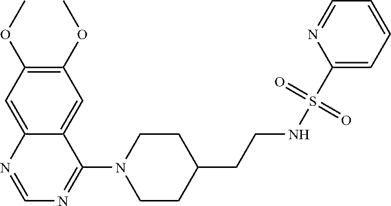 [SMILES: COc1cc2ncnc(N3CCC(CCNS(=O)(=O)c4ccccn4)CC3)c2cc1OC] |
| 156 | N-(4-(2-((6,7-dimethoxyquinazolin-4-yl)amino)ethyl)phenyl) sulfamide | 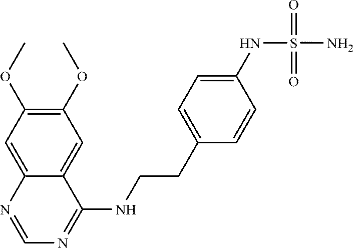 [SMILES: COc1cc2ncnc(NCCc3ccc(NS(N)(=O)=O)cc3)c2cc1OC] |
| 157 | 5-(1-(6,7-dimethoxyquinazolin-4-yl)-1,2,3,6-tetrahydropyridin-4-yl)benzo[c][1,2] oxaborol-1(3H)-ol | 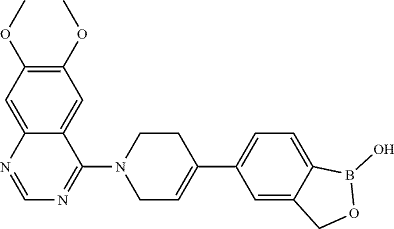 [SMILES: COc1cc2ncnc(N3CC=C(c4ccc5c(c4)COB5O)CC3)c2cc1OC] |
| 158 | 8-(quinazolin-4-yl)-2,8-diazaspiro [4.5] decane -2 -sulfonamide | 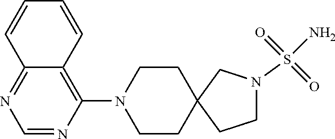 [SMILES: NS(=O)(=O)N1CCC2(CCN(c3ncnc4ccccc34)CC2)C1] |
| 159 | 3-(1-(6,7-dimethoxyquinazolin-4-yl)piperidin-4-yl)-N-hydroxypropanamide | 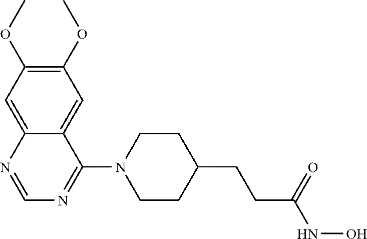 [SMILES: COc1cc2ncnc(N3CCC(CCC(=O)NO)CC3)c2cc1OC] |
| 160 | 8-(6,7-dimethoxyquinolin-4-yl)-2,8-diazaspiro[4.5]decane-2-sulfonamide | 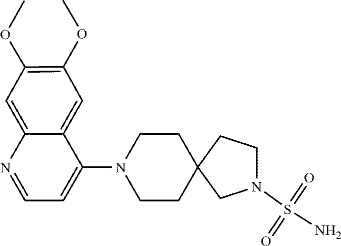 [SMILES: COc1cc2nccc(N3CCC4(CC3)CCN(S(N)(=O)=O)C4)c2cc1OC] |
| 161 | 4-(((6,7-dimethoxyquinazolin-4-yl)amino)methyl)benzene sulfonic acid | 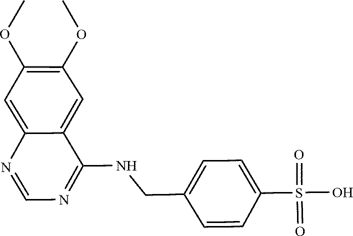 [SMILES: COc1cc2ncnc(NCc3ccc(S(=O)(=O)O)cc3)c2cc1OC] |
| 162 | N-(4-((6,7-dimethoxyquinolin-4-yl)oxy)phenyl)-1,1,1-trifluoromethanesulfonamide | 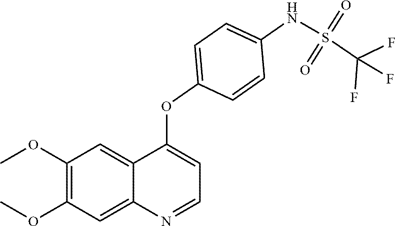 [SMILES: COc1cc2nccc(Oc3ccc(NS(=O)(=O)C(F)(F)F)cc3)c2cc1OC] |
| 163 | N-methyl-4-(4-(2-(sulfamoylamino)ethyl)piperidin-1 -yl)quinazoline-7-carboxamide | 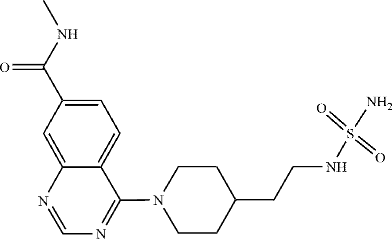 [SMILES: CNC(=O)c1ccc2c(N3CCC(CCNS(N)(=O)=O)CC3)ncnc2c1] |
| 164 | 2-(1-(6,7-dimethoxyquinazolin-4-yl)piperidin-4-yl)ethane-1-sulfonamide | 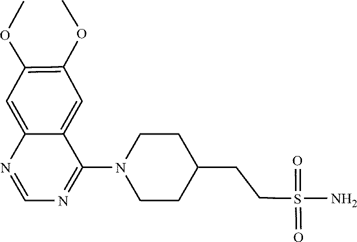 [SMILES: COc1cc2ncnc(N3CCC(CCS(N)(=O)=O)CC3)c2cc1OC] |
| 165 | N-(4-((6,7-dimethoxy-4-oxoquinazolin-3 (4H)-yl)methyl)phenyl) sulfamide | 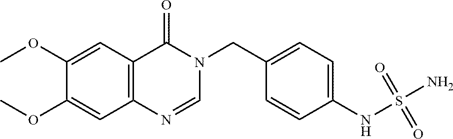 [SMILES: COc1cc2ncn(Cc3ccc(NS(N)(=O)=O)cc3)c(=O)c2cc1OC] |
| 166 | N-(4-(5,6-dimethoxy-1H-indazol-1-yl)benzyl)sulfamide | 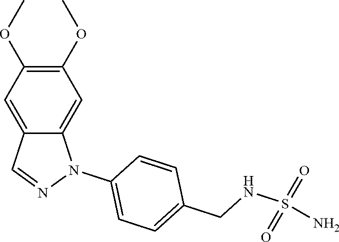 [SMILES: COc1cc2cnn(-c3ccc(CNS(N)(=O)=O)cc3)c2cc1OC] |
| 167 | 8-(3 -cyanoquinolin-4 -yl) -2, 8-diazaspiro [4.5] decane -2 -sulfonamide | 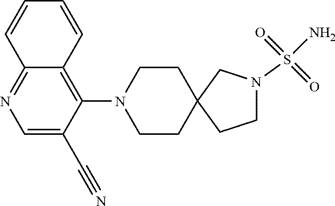 [SMILES: N#Cc1cnc2ccccc2c1N1CCC2(CC1)CCN(S(N)(=O)=O)C2] |
| 168 | 2-(1-(6,7-dimethoxyquinazolin-4-yl)piperidin-4-yl)ethan-1-amino(N-methyl)sulfonamide | 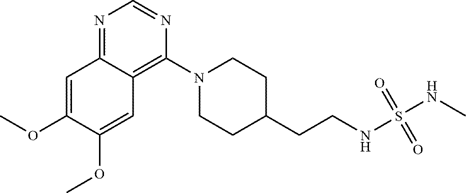 |
| 169 | N-(2-(1-(6-chloro-7-methoxyquinazolin-4-yl)piperidin-4-yl)ethyl)sulfamide | 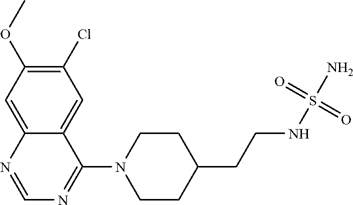 [SMILES: COc1cc2ncnc(N3CCC(CCNS(N)(=O)=O)CC3)c2cc1Cl] |
| 170 | N-(2-(1-([1,3]dioxolo[4,5-g] quinazolin-8 -yl)piperidin-4-yl)ethyl)sulfamide | 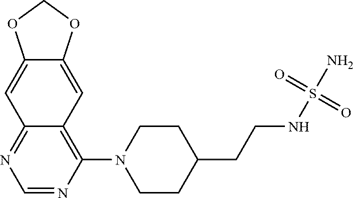 [SMILES: NS(=O)(=O)NCCC1CCN(c2ncnc3cc4c(cc23)OCO4)CC1] |
| 171 | 3-(1-(6,7-dimethoxyquinazolin-4-yl)piperidin-4-yl)propanoic acid | 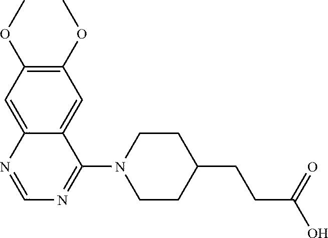 [SMILES: COc1cc2ncnc(N3CCC(CCC(=O)O)CC3)c2cc1OC] |
| 172 | N-(1-(6,7-dimethoxyquinolin-4-yl)piperidin-4-yl)(N-ethylamino)sulfonamide | 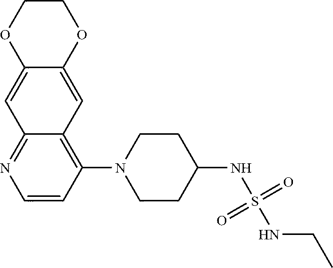 [SMILES: CCNS(=O)(=O)NC1CCN(c2ccnc3cc4c(cc23)OCCO4)CC1] |
| 173 | N-(4-((6,7-dimethoxy-2-oxo-3,4-dihydroquinolin-1 (2H)-yl)methyl)phenyl) sulfamide | 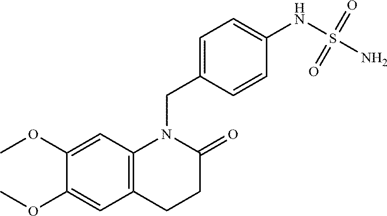 [SMILES: COc1cc2c(cc1OC)N(Cc1ccc(NS(N)(=O)=O)cc1)C(=O)CC2] |
| 174 | N-(4-((6,7-dimethoxyquinolin-4-yl)amino)phenyl)-1,1,1-trifluoromethanesulfonamide | 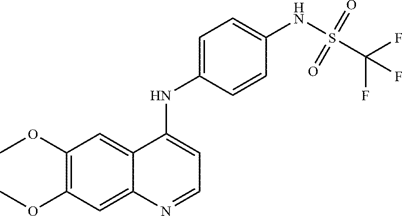 [SMILES: COc1cc2nccc(Nc3ccc(NS(=O)(=O)C(F)(F)F)cc3)c2cc1OC] |
| 175 | N-(4-((3-cyano-6,7-dimethoxyquinolin-4-yl)amino)phenyl)sulfamide | 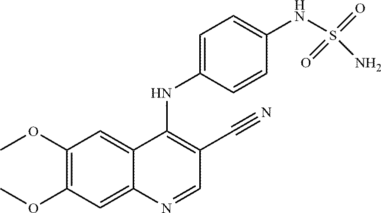 [SMILES: COc1cc2ncc(C#N)c(Nc3ccc(NS(N)(=O)=O)cc3)c2cc1OC] |
| 176 | N-(5 -((6,7-dimethoxyquinolin-4-yl)oxy)pyridin-2-yl)sulfamide | 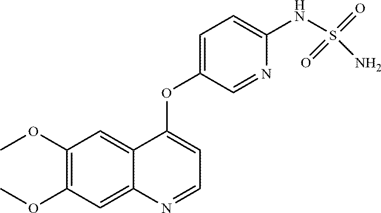 [SMILES: COc1cc2nccc(Oc3ccc(NS(N)(=O)=O)nc3)c2cc1OC] |
| 177 | 2-(1-(6,7-dimethoxyquinolin-4-yl)piperidin-4-yl)ethan-1-amino(N-trifluoroethyl) sulfonamide | 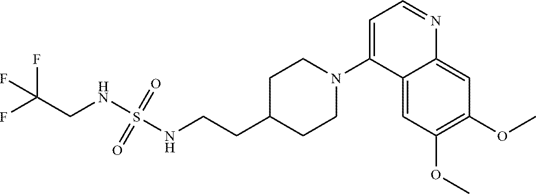 [SMILES: COc1cc2nccc(N3CCC(CCNS(=O)(=O)NCC(F)(F)F)CC3)c2cc1OC] |
| 178 | N-((1-(6,7-dimethoxyquinoline-4-carbonyl)piperidin-4-yl)methyl)sulfamide | 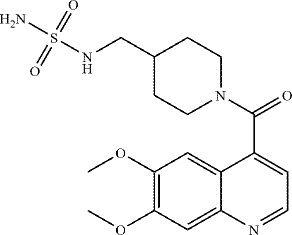 [SMILES: COc1cc2nccc(C(=O)N3CCC(CNS(N)(=O)=O)CC3)c2cc1OC] |
| 179 | N-(4-(((6,7-dimethoxyquinazolin-4-yl)oxy)methyl)phenyl)sulfamide | 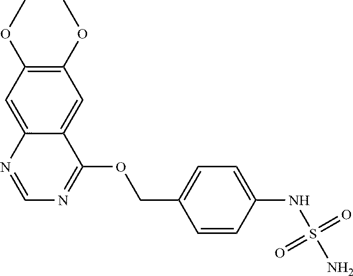 [SMILES: COc1cc2ncnc(OCc3ccc(NS(N)(=O)=O)cc3)c2cc1OC] |
| 180 | N-(4-(((6,7-dimethoxyquinolin-4-yl)amino)methyl)phenyl)sulfamide | 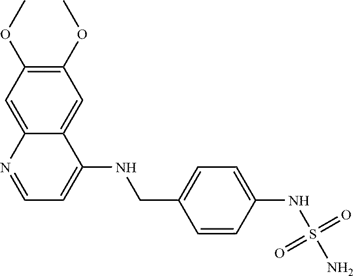 [SMILES: COc1cc2nccc(NCc3ccc(NS(N)(=O)=O)cc3)c2cc1OC] |
| 181 | N-(4-(1-((6,7-dimethoxyquinolin-4-yl)amino)ethyl)phenyl)sulfamide | 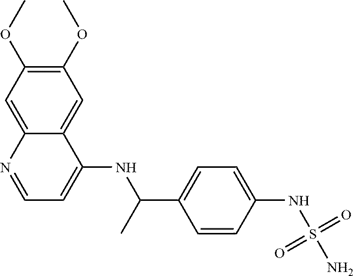 [SMILES: COc1cc2nccc(NC(C)c3ccc(NS(N)(=O)=O)cc3)c2cc1OC] |
| 182 | 4-(((6,7-dimethoxyquinazolin-4-yl)amino)methyl)benzene sulfonam ide | 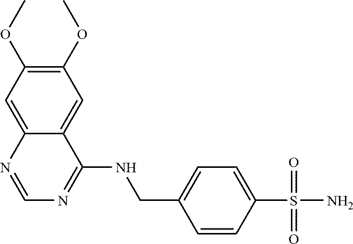 [SMILES: COc1cc2ncnc(NCc3ccc(S(N)(=O)=O)cc3)c2cc1OC] |
| 183 | 2-(1-(6,7-dimethoxyquinolin-4-yl)piperidin-4-yl)ethan-1-amino(N-phenyl)sulfonamide | 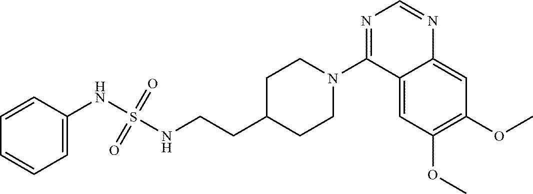 [SMILES: COc1cc2ncnc(N3CCC(CCNS(=O)(=O)Nc4ccccc4)CC3)c2cc1OC] |
| 184 | N-(2-( 1 -(7-fluoro-6-methoxyquinazolin-4-yl)piperidin-4-yl)ethyl)sulfamide | 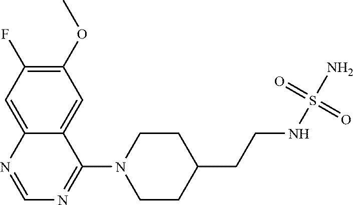 [SMILES: COc1cc2c(N3CCC(CCNS(N)(=O)=O)CC3)ncnc2cc1F] |
| 185 | N-(2-(1-(7-chloro-6-methoxyquinazolin-4-yl)piperidin-4-yl)ethyl)sulfamide | 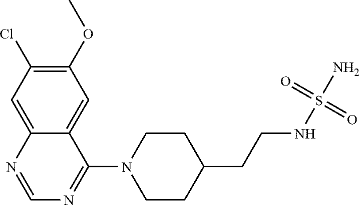 [SMILES: COc1cc2c(N3CCC(CCNS(N)(=O)=O)CC3)ncnc2cc1Cl] |
| 186 | N-(4-(5,6-dimethoxy-1H-indol-1-yl)benzyl)sulfamide | 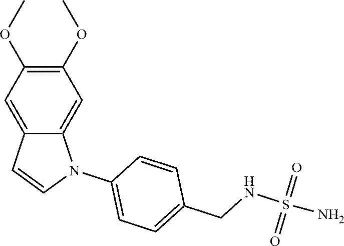 [SMILES: COc1cc2ccn(-c3ccc(CNS(N)(=O)=O)cc3)c2cc1OC] |
| 187 | 8-(6,7-dimethoxyquinolin-4-yl)-2,8-diazaspiro[4.5]decan-1-one | 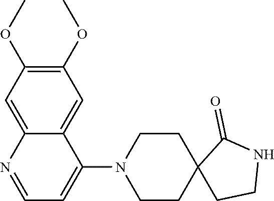 [SMILES: COc1cc2nccc(N3CCC4(CCNC4=O)CC3)c2cc1OC] |
| 188 | N-(6,7-dimethoxyquinazolin-4-yl)-4-(sulfamoylamino)benzamide | 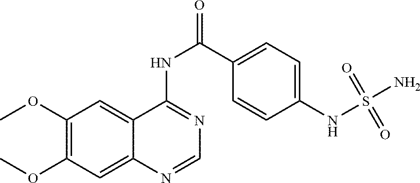 [SMILES: COc1cc2ncnc(NC(=O)c3ccc(NS(N)(=O)=O)cc3)c2cc1OC] |
| 189 | N-(2-(1-(6-methoxypyrido[3,4-d]pyrimidin-4-yl)piperidin-4-yl)ethyl)sulfamide | 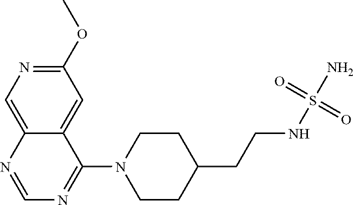 [SMILES: COc1cc2c(N3CCC(CCNS(N)(=O)=O)CC3)ncnc2cn1] |
| 190 | N-(4-((6,7-dimethoxyquinazolin-4-yl)(methyl)amino)phenyl)sulfamid e | 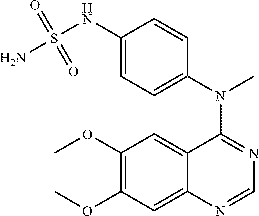 [SMILES: COc1cc2ncnc(N(C)c3ccc(NS(N)(=O)=O)cc3)c2cc1OC] |
| 191 | N-(4-(((6,7-dimethoxyquinolin-4-yl)oxy)methyl)phenyl) sulfamide | 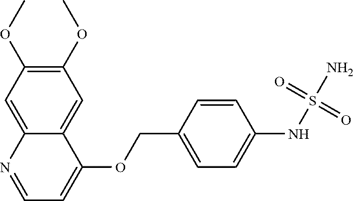 [SMILES: COc1cc2nccc(OCc3ccc(NS(N)(=O)=O)cc3)c2cc1OC] |
| 192 | 4-(4-(2-(sulfamoylamino)ethyl)piperidin-1-yl)quinoline-6-carboxamide | 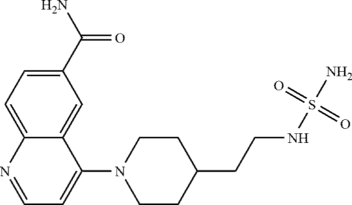 [SMILES: NC(=O)c1ccc2nccc(N3CCC(CCNS(N)(=O)=O)CC3)c2c1] |
| 193 | 7-(1-methyl-1H-imidazo[4,5-b]pyridin-7-yl)-3,4-dihydroisoquinoline-2(1H)-sulfonamide | 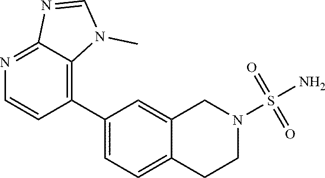 [SMILES: Cn1cnc2nccc(-c3ccc4c(c3)CN(S(N)(=O)=O)CC4)c21] |
| 194 | N-(2-(1-(6-(trifluoromethoxy)quinazolin-4-yl)piperidin-4-yl)ethyl)sulfamide | 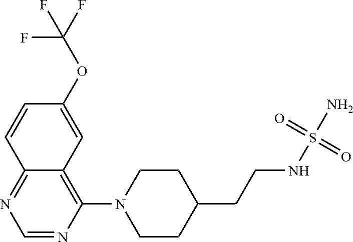 [SMILES: NS(=O)(=O)NCCC1CCN(c2ncnc3ccc(OC(F)(F)F)cc23)CC1] |
| 195 | 2-(2-(1-(6,7-dimethoxyquinazolin-4-yl)piperidin-4-yl)ethyl)-2H-tetrazol-5 -amine | 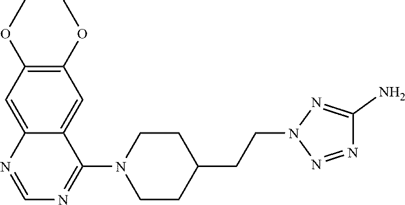 [SMILES: COc1cc2ncnc(N3CCC(CCn4nnc(N)n4)CC3)c2cc1OC] |
| 196 | 4-(((6,7-dimethoxyquinazolin-4-yl)amino)methyl)-N-hydroxybenzamide | 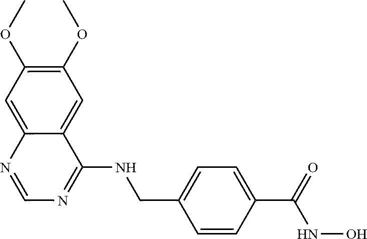 [SMILES: COc1cc2ncnc(NCc3ccc(C(=O)NO)cc3)c2cc1OC] |
| 197 | N-(4-((6,7-dimethoxyquinolin-4-yl)oxy)-3,5-difluorophenyl)sulfamide | 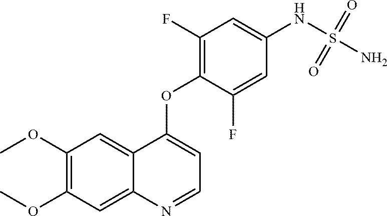 [SMILES: COc1cc2nccc(Oc3c(F)cc(NS(N)(=O)=O)cc3F)c2cc1OC] |
| 198 | N-(2-( 1 -(7-methoxyquinazolin-4-yl)piperidin-4-yl)ethyl)sulfamide | 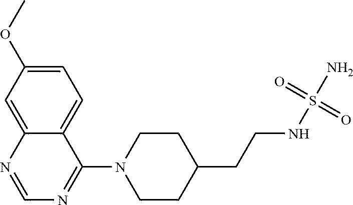 [SMILES: COc1ccc2c(N3CCC(CCNS(N)(=O)=O)CC3)ncnc2c1] |
| 199 | N-(2-(1-(6-methoxyquinazolin-4-yl)piperidin-4-yl)ethyl)sulfamide | 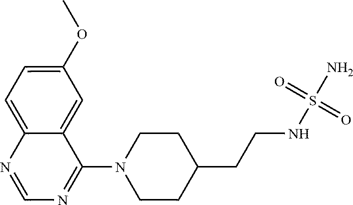 [SMILES: COc1ccc2ncnc(N3CCC(CCNS(N)(=O)=O)CC3)c2c1] |
| 200 | N-(N-(2-(1-(6,7-dimethoxyquinazolin-4-yl)piperidin-4-yl)ethyl)sulfamoyl)acetamide | 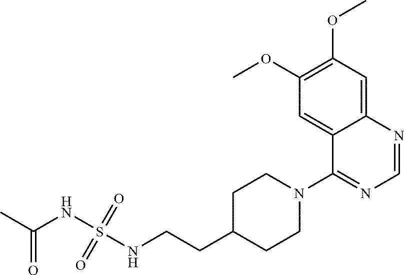 [SMILES: COc1cc2ncnc(N3CCC(CCNS(=O)(=O)NC(C)=O)CC3)c2cc1OC] |
| 201 | N-(4-((6,7-dimethoxyquinolin-4-yl)oxy)-3 -fluorophenyl)sulfamide | 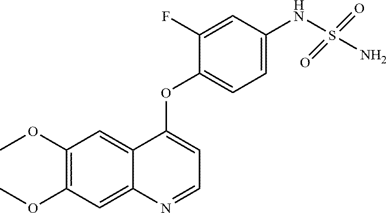 [SMILES: COc1cc2nccc(Oc3ccc(NS(N)(=O)=O)cc3F)c2cc1OC] |
| 202 | 5-(6,7-dimethoxyquinolin-4-yl)hexahydropyrrolo[3,4-c]pyrrole-2(1H)-sulfonamide | 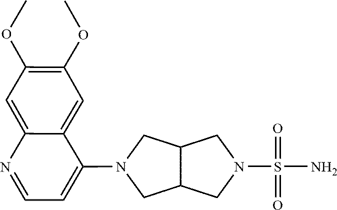 [SMILES: COc1cc2nccc(N3CC4CN(S(N)(=O)=O)CC4C3)c2cc1OC] |
| 203 | N-(N-(2-(1-(6,7-dimethoxyquinolin-4-yl)piperidin-4-yl)ethyl)sulfamoyl)acetamide | 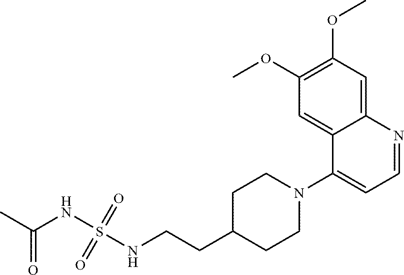 [SMILES: COc1cc2nccc(N3CCC(CCNS(=O)(=O)NC(C)=O)CC3)c2cc1OC] |
| 204 | N-(2-(1-(7-methoxy-1,6-naphthyridin-4-yl)piperidin-4-yl)ethyl)sulfamide | 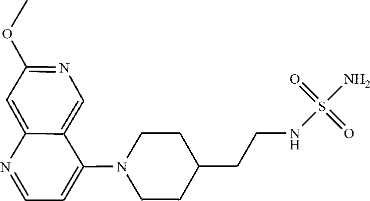 [SMILES: COc1cc2nccc(N3CCC(CCNS(N)(=O)=O)CC3)c2cn1] |
| 205 | N-(2-(1-(6-isopropoxy-7-methoxyquinazolin-4-yl)piperidin-4-yl)ethyl)sulfamide | 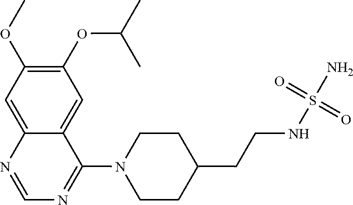 [SMILES: COc1cc2ncnc(N3CCC(CCNS(N)(=O)=O)CC3)c2cc1OC(C)C] |
| 206 | N-(2-(1-(6-fluoro-7-methoxyquinazolin-4-yl)piperidin-4-yl)ethyl)sulfamide | 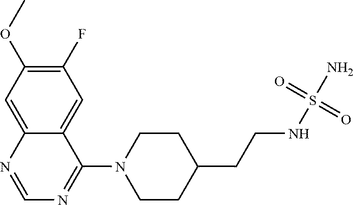 [SMILES: COc1cc2ncnc(N3CCC(CCNS(N)(=O)=O)CC3)c2cc1F] |
| 207 | 2-(4-((6,7-dimethoxyquinolin-4-yl)amino)phenyl)-N-hydroxyacetamide | 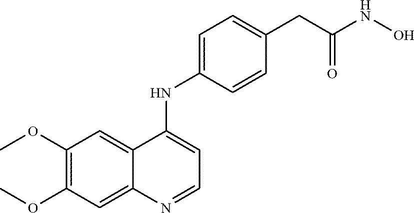 [SMILES: COc1cc2nccc(Nc3ccc(CC(=O)NO)cc3)c2cc1OC] |
| 208 | N-(2-(1-(6,7-dimethoxyquinazolin-4-yl)piperidin-4-yl)ethyl)-1,1-difluoromethanesulfonamide | 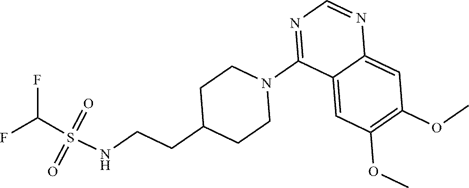 [SMILES: COc1cc2ncnc(N3CCC(CCNS(=O)(=O)C(F)F)CC3)c2cc1OC] |
| 209 | N-(2-(1-(7-(trifluoromethoxy)quinazolin-4-yl)piperidin-4-yl)ethyl)sulfamide | 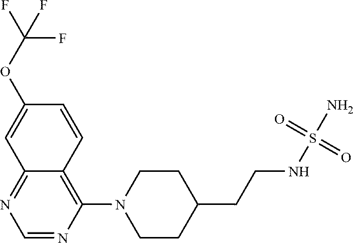 [SMILES: NS(=O)(=O)NCCC1CCN(c2ncnc3cc(OC(F)(F)F)ccc23)CC1] |
| 210 | N-(4-(5,6-dimethoxy-1H-indazol-1-yl)phenethyl)sulfamide | 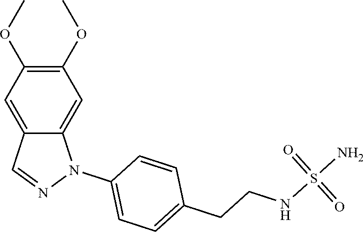 [SMILES: COc1cc2cnn(-c3ccc(CCNS(N)(=O)=O)cc3)c2cc1OC] |
| 211 | N-(2-(1-( 1-phenyl-1H-imidazo[4,5-b]pyridin-7-yl)piperidin-4-yl)ethyl)sulfamide | 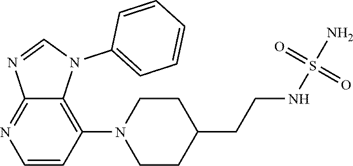 [SMILES: NS(=O)(=O)NCCC1CCN(c2ccnc3ncn(-c4ccccc4)c23)CC1] |
| 212 | N-cyclopropyl-N-((1-(6,7-dimethoxyquinolin-4-yl)piperidin-4-yl)methyl)sulfamide | 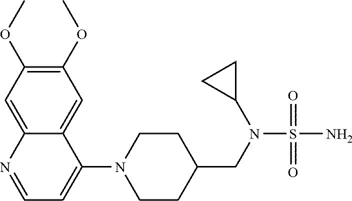 [SMILES: COc1cc2nccc(N3CCC(CN(C4CC4)S(N)(=O)=O)CC3)c2cc1OC] |
| 213 | N-(3-((6,7-dimethoxyquinolin-4-yl)oxy)cyclopentyl)sulfamide | 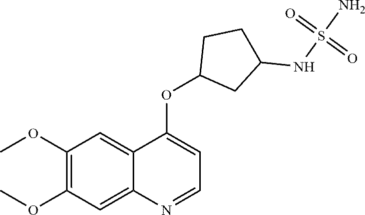 [SMILES: COc1cc2nccc(OC3CCC(NS(N)(=O)=O)C3)c2cc1OC] |
| 214 | 6-(6,7-dimethoxyquinolin-4-yl)-2,6-diazaspiro[3.3]heptane-2-sulfonamide | 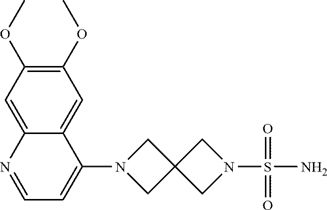 [SMILES: COc1cc2nccc(N3CC4(C3)CN(S(N)(=O)=O)C4)c2cc1OC] |
| 215 | N-(2-(1-([1,3]dioxolo[4,5-g]quinolin-8-yl)piperidin-4-yl)ethyl)sulfamide | 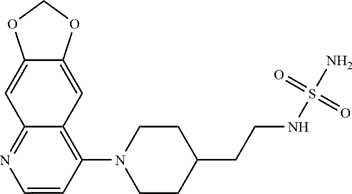 [SMILES: NS(=O)(=O)NCCC1CCN(c2ccnc3cc4c(cc23)OCO4)CC1] |
| 216 | N-(2-(1-(3-cyano-6-methoxy-1,7-naphthyridin-4-yl)piperidin-4-yl)ethyl)sulfamide | 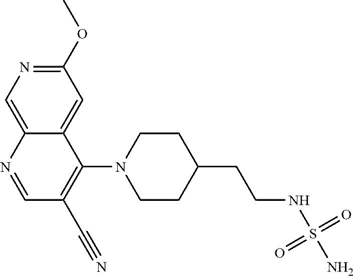 [SMILES: COc1cc2c(N3CCC(CCNS(N)(=O)=O)CC3)c(C#N)cnc2cn1] |
| 217 | N-(4-(5,6-dimethoxy-1H-benzo[d]imidazol-1-yl)phenethyl)sulfamide | 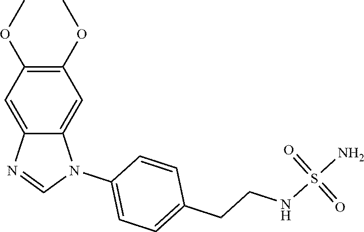 [SMILES: COc1cc2ncn(-c3ccc(CCNS(N)(=O)=O)cc3)c2cc1OC] |
| 218 | N-(2-(1-(8-methoxy-7-methyl-7H-purin-6-yl)piperidin-4-yl)ethyl)sulfamide | 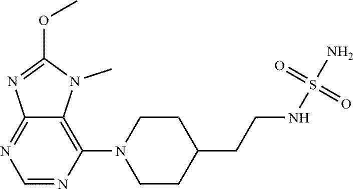 [SMILES: COc1nc2ncnc(N3CCC(CCNS(N)(=O)=O)CC3)c2n1C] |
| 219 | N-(2-(1-(6,7-dimethoxyquinazolin-4-yl)piperidin-4-yl)ethyl)-N-hydroxyformamide | 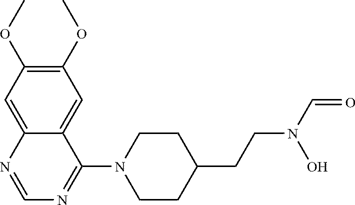 [SMILES: COc1cc2ncnc(N3CCC(CCN(O)C=O)CC3)c2cc1OC] |
| 220 | N-(2-(1-(6-fluoro-7-methoxyquinolin-4-yl)piperidin-4-yl)ethyl)sulfamide | 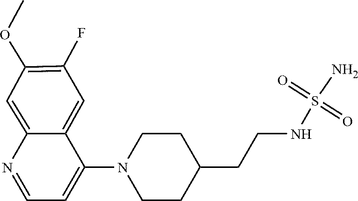 [SMILES: COc1cc2nccc(N3CCC(CCNS(N)(=O)=O)CC3)c2cc1F] |
| 221 | N-(2-(1-(7-cyano-[1,3]dioxolo[4,5-g]quinolin-8-yl)piperidin-4-yl)ethyl)sulfamide | 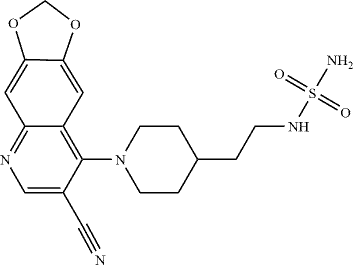 [SMILES: N#Cc1cnc2cc3c(cc2c1N1CCC(CCNS(N)(=O)=O)CC1)OCO3] |
| 222 | 3-(1-(6,7-dimethoxyquinazolin-4-yl)piperidin-4-yl)propanamide | 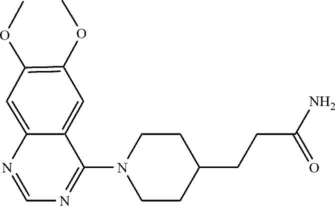 [SMILES: COc1cc2ncnc(N3CCC(CCC(N)=O)CC3)c2cc1OC] |
| 223 | 2-((1-(6,7-dimethoxyquinolin-4-yl)piperidin-4-yl)methyl)-1,2,6-thiadiazinane 1,1-dioxide | 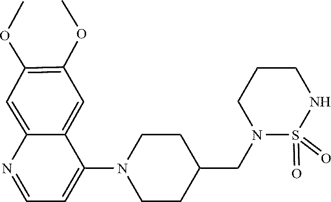 [SMILES: COc1cc2nccc(N3CCC(CN4CCCNS4(=O)=O)CC3)c2cc1OC] |
| 224 | N-(5-((7-methoxyquinolin-4-yl)oxy)pyrimidin-2-yl)sulfamide | 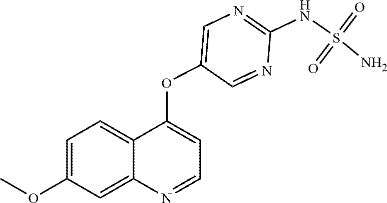 [SMILES: COc1ccc2c(Oc3cnc(NS(N)(=O)=O)nc3)ccnc2c1] |
| 225 | N-(4-(6,7-dimethoxyquinolin-4-yl)benzyl)sulfamide | 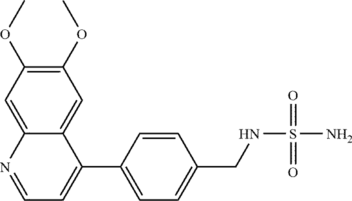 [SMILES: COc1cc2nccc(-c3ccc(CNS(N)(=O)=O)cc3)c2cc1OC] |
| 226 | N-(4-(7-methoxyquinazolin-4-yl)benzyl)sulfamide | 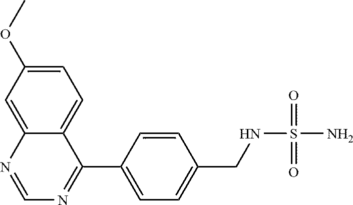 [SMILES: COc1ccc2c(-c3ccc(CNS(N)(=O)=O)cc3)ncnc2c1] |
| 227 | N-(4-((7-methoxypyrido[2,3-d]pyrimidin-4-yl)oxy)phenyl)sulfamide | 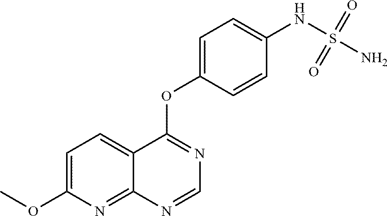 [SMILES: COc1ccc2c(Oc3ccc(NS(N)(=O)=O)cc3)ncnc2n1] |
| 228 | 2-(4-(((6,7-dimethoxyquinazolin-4-yl)amino)methyl)phenyl)-1,2,5-thiadiazolidine 1,1-dioxide | 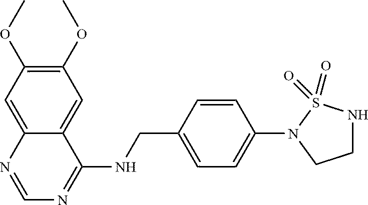 [SMILES: COc1cc2ncnc(NCc3ccc(N4CCNS4(=O)=O)cc3)c2cc1OC] |
| 229 | 7-(3-fluoro-6,7-dimethoxyquinolin-4-yl)-3,4-dihydroisoquinoline-2(1H)-sulfonamide | 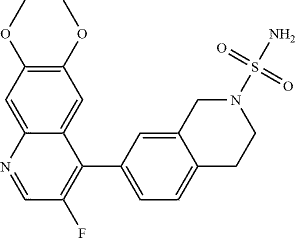 [SMILES: COc1cc2ncc(F)c(-c3ccc4c(c3)CN(S(N)(=O)=O)CC4)c2cc1OC] |
| 230 | 4-(((7-methoxyquinazolin-4-yl)amino)methyl)benzene sulfonam ide | 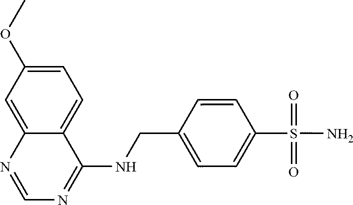 [SMILES: COc1ccc2c(NCc3ccc(S(N)(=O)=O)cc3)ncnc2c1] |
| 231 | N-(4-((7-methoxy-1,8-naphthyridin-4-yl)amino)phenyl)sulfamide | 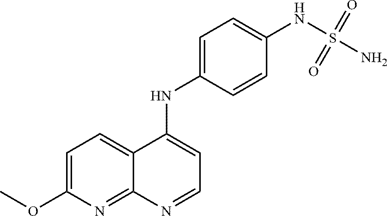 [SMILES: COc1ccc2c(Nc3ccc(NS(N)(=O)=O)cc3)ccnc2n1] |
| 232 | N-(2-(1-(5,6,7,8-tetrahydropyrido[3,4-d]pyrimidin-4-yl)piperidin-4-yl)ethyl)sulfamide | 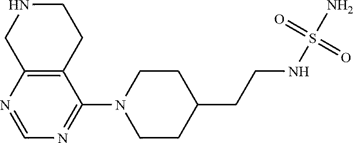 [SMILES: NS(=O)(=O)NCCC1CCN(c2ncnc3c2CCNC3)CC1] |
| 233 | N-(2-(1-(7-benzyl-5,6,7,8-tetrahydropyrido[3,4-d]pyrimidin-4-yl)piperidin-4-yl)ethyl)sulfamide | 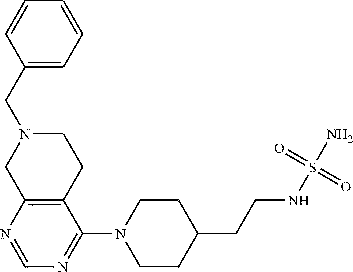 [SMILES: NS(=O)(=O)NCCC1CCN(c2ncnc3c2CCN(Cc2ccccc2)C3)CC1] |
| 234 | N-(4-((6,7-dimethoxyquinazolin-4-yl)amino)benzyl)sulfamide | 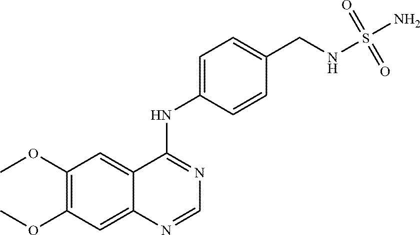 [SMILES: COc1cc2ncnc(Nc3ccc(CNS(N)(=O)=O)cc3)c2cc1OC] |
| 235 | N-(4-(3 -cyano-6-methoxy-1,7-naphthyridin-4-yl)benzyl)sulfamide | 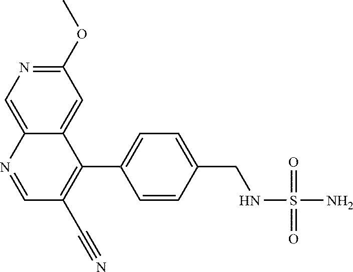 [SMILES: COc1cc2c(-c3ccc(CNS(N)(=O)=O)cc3)c(C#N)cnc2cn1] |
| 236 | N-(4-(1-((6-fluoro-7-methoxyquinazolin-4-yl)amino)ethyl)phenyl)sulfamide | 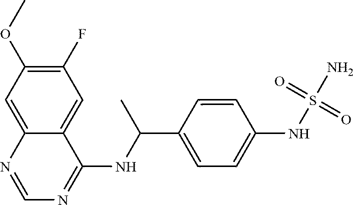 [SMILES: COc1cc2ncnc(NC(C)c3ccc(NS(N)(=O)=O)cc3)c2cc1F] |
| 237 | 7-(3-cyano-6-methoxyquinolin-4-yl)-3,4-dihydroisoquinoline-2(1H)-sulfonamide | 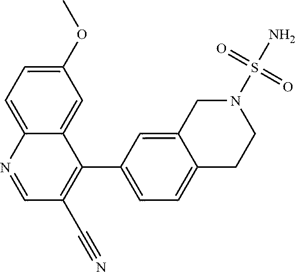 [SMILES: COc1ccc2ncc(C#N)c(-c3ccc4c(c3)CN(S(N)(=O)=O)CC4)c2c1] |
| 238 | 8-(3-cyano-6-methoxyquinolin-4-yl)-2,8-diazaspiro[4.5]decane-2-sulfonamide | 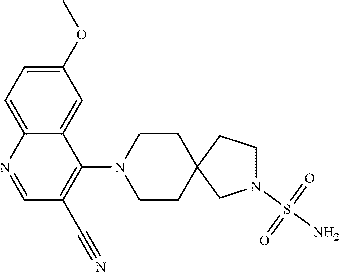 [SMILES: COc1ccc2ncc(C#N)c(N3CCC4(CC3)CCN(S(N)(=O)=O)C4)c2c1] |
| 239 | N-(4-(6-fluoro-7-methoxyquinolin-4-yl)benzyl)sulfamide | 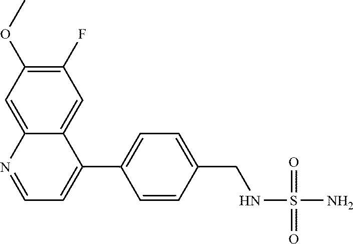 [SMILES: COc1cc2nccc(-c3ccc(CNS(N)(=O)=O)cc3)c2cc1F] |
| 240 | N-(4-(3-cyano-6-methoxyquinolin-4-yl)benzyl)sulfamide | 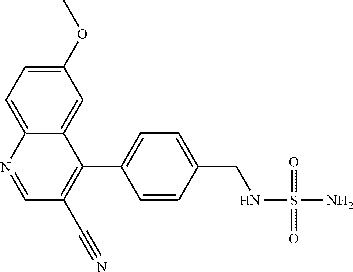 [SMILES: COc1ccc2ncc(C#N)c(-c3ccc(CNS(N)(=O)=O)cc3)c2c1] |
| 241 | N-(4-(3-cyano-6,7-dimethoxyquinolin-4-yl)benzyl)sulfamide | 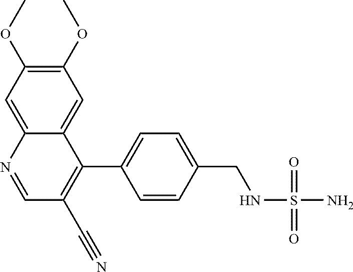 [SMILES: COc1cc2ncc(C#N)c(-c3ccc(CNS(N)(=O)=O)cc3)c2cc1OC] |
| 242 | N-(4-(pyrido[2,3-d]pyrimidin-4-ylamino)phenyl)sulfamide | 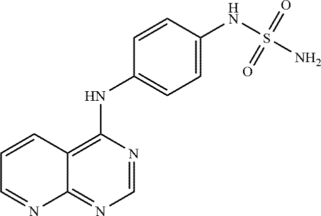 [SMILES: NS(=O)(=O)Nc1ccc(Nc2ncnc3ncccc23)cc1] |
| 243 | 2-(4-((6,7-dimethoxyquinazolin-4-yl)amino)phenyl)-1,2,5-thiadiazolidine 1,1-dioxide | 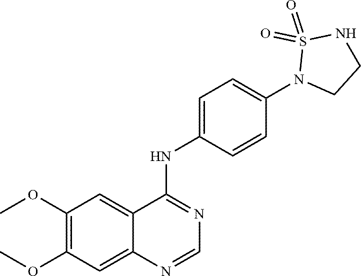 [SMILES: COc1cc2ncnc(Nc3ccc(N4CCNS4(=O)=O)cc3)c2cc1OC] |
| 244 | N-(4-(3-cyano-7-methoxyquinolin-4-yl)benzyl)sulfamide | 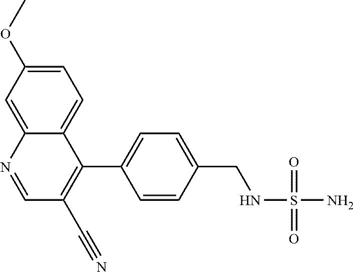 [SMILES: COc1ccc2c(-c3ccc(CNS(N)(=O)=O)cc3)c(C#N)cnc2c1] |
| 245 | N-(2-(1-(3-cyano-7-methoxy-1,6-naphthyridin-4-yl)piperidin-4-yl)ethyl)sulfamide | 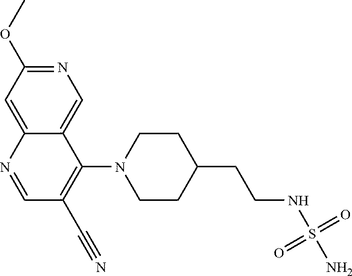 [SMILES: COc1cc2ncc(C#N)c(N3CCC(CCNS(N)(=O)=O)CC3)c2cn1] |
| 246 | N-(4-(cyclopropyl((6,7-dimethoxyquinazolin-4-yl)amino)methyl)phenyl)sulfamide | 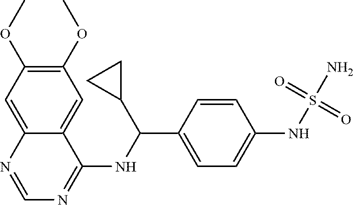 [SMILES: COc1cc2ncnc(NC(c3ccc(NS(N)(=O)=O)cc3)C3CC3)c2cc1OC] |
| 247 | 4-(((6-methoxypyrido[3,4-d]pyrimidin-4-yl)amino)methyl)benzenesulfonam ide | 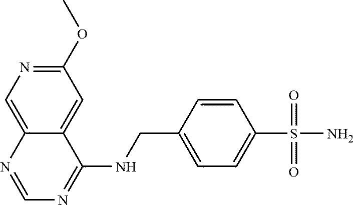 [SMILES: COc1cc2c(NCc3ccc(S(N)(=O)=O)cc3)ncnc2cn1] |
| 248 | 8-(3-cyano-7-methoxyquinolin-4-yl)-2,8-diazaspiro[4.5]decane-2-sulfonamide | 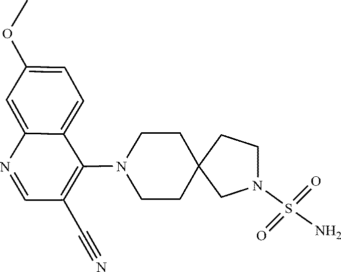 [SMILES: COc1ccc2c(N3CCC4(CC3)CCN(S(N)(=O)=O)C4)c(C#N)cnc2c1] |
| 249 | 7-(3-cyano-7-methoxyquinolin-4-yl)-3,4-dihydroisoquinoline-2(1H)-sulfonamide | 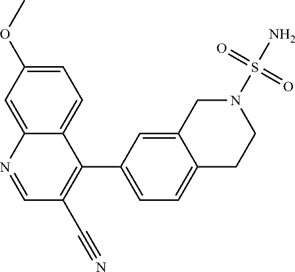 [SMILES: COc1ccc2c(-c3ccc4c(c3)CN(S(N)(=O)=O)CC4)c(C#N)cnc2c1] |
| 250 | 8-(7-fluoroquinazolin-4-yl)-2,8-diazaspiro [4.5] decane -2-sulfonamide | 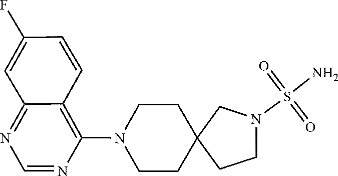 [SMILES: NS(=O)(=O)N1CCC2(CCN(c3ncnc4cc(F)ccc34)CC2)C1] |
| 251 | N-((1-(6,7-dimethoxyquinazolin-4-yl)piperidin-4-yl)methyl)-1H-benzo[d]imidazol-2-amine | 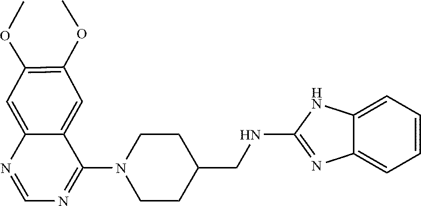 [SMILES: COc1cc2ncnc(N3CCC(CNc4nc5ccccc5[nH]4)CC3)c2cc1OC] |
| 252 | 1-((1-(6,7-dimethoxyquinazolin-4-yl)piperidin-4-yl)methyl)-1H-benzo[d]imidazol-2-amine | 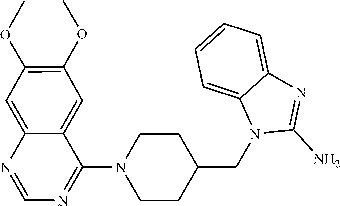 [SMILES: COc1cc2ncnc(N3CCC(Cn4c(N)nc5ccccc54)CC3)c2cc1OC] |
| 253 | (R)-N-(4-(1-((6,7-dimethoxyquinazolin-4-yl)amino)ethyl)phenyl)sulfamide | 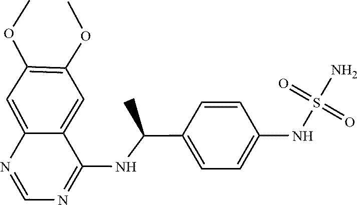 [SMILES: COc1cc2ncnc(N[C@@H](C)c3ccc(NS(N)(=O)=O)cc3)c2cc1OC] |
| 254 | (S)-N-(4-(1-((6,7-dimethoxyquinazolin-4-yl)amino)ethyl)phenyl)sulfamide | 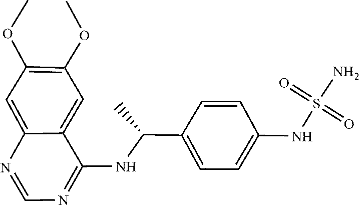 [SMILES: COc1cc2ncnc(N[C@H](C)c3ccc(NS(N)(=O)=O)cc3)c2cc1OC] |
| 255 | N-(4-(((6,7-dimethoxyquinolin-4-yl)(methyl)amino)methyl)phenyl)s ulfamide | 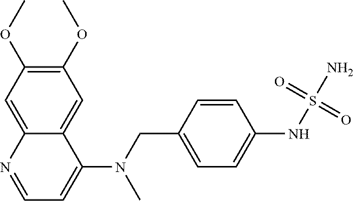 [SMILES: COc1cc2nccc(N(C)Cc3ccc(NS(N)(=O)=O)cc3)c2cc1OC] |
| 256 | 8-(3-cyano-6-methoxy-1,7-naphthyridin-4-yl)-2,8-diazaspiro [4.5] decane -2-sulfonamide | 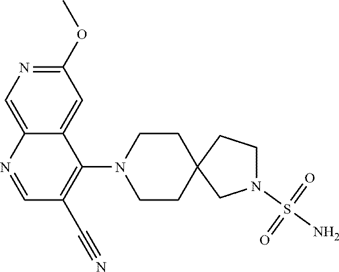 [SMILES: COc1cc2c(N3CCC4(CC3)CCN(S(N)(=O)=O)C4)c(C#N)cnc2cn1] |
| 257 | 7-(3-cyano-6-methoxy-1,7-naphthyridin-4-yl)-3,4-dihydroisoquinoline-2(1H)-sulfonamide | 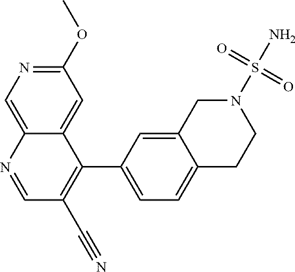 [SMILES: COc1cc2c(-c3ccc4c(c3)CN(S(N)(=O)=O)CC4)c(C#N)cnc2cn1] |
| 258 | N-(4-((6,7-dimethoxyquinolin-4-yl)oxy)-2,5-difluorophenyl)sulfamide | 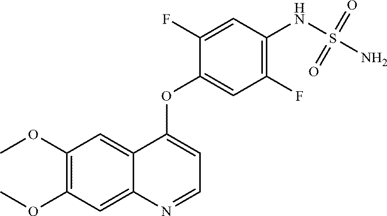 [SMILES: COc1cc2nccc(Oc3cc(F)c(NS(N)(=O)=O)cc3F)c2cc1OC] |
| 259 | 4-(((6-fluoro-7-methoxyquinazolin-4-yl)amino)methyl)benzenesulfonam ide | 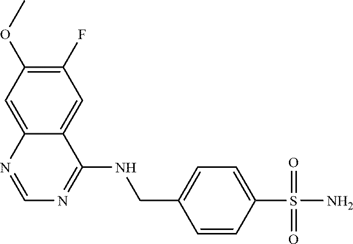 [SMILES: COc1cc2ncnc(NCc3ccc(S(N)(=O)=O)cc3)c2cc1F] |
| 260 | 7-(4-chloro-6,7-dimethoxyquinolin-3-yl)-3,4-dihydroisoquinoline-2(1H)-sulfonamide | 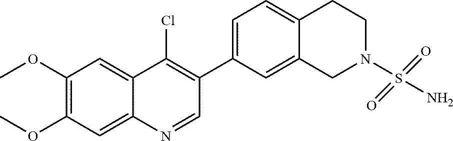 [SMILES: COc1cc2ncc(-c3ccc4c(c3)CN(S(N)(=O)=O)CC4)c(Cl)c2cc1OC] |
| 261 | N-(4-((6,7-dimethoxyquinolin-4-yl)oxy)phenyl)cyclobutanesulfona mide | 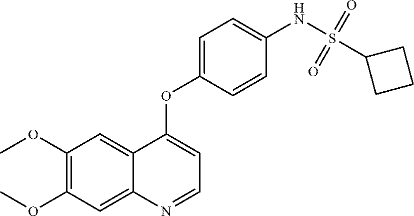 [SMILES: COc1cc2nccc(Oc3ccc(NS(=O)(=O)C4CCC4)cc3)c2cc1OC] |
| 262 | 7-(3-cyano-7-(difluoromethoxy)quinolin-4-yl)-3,4-dihydroisoquinoline-2(1H)-sulfonamide | 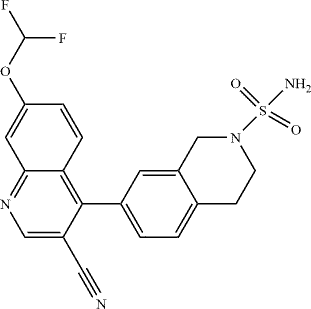 [SMILES: N#Cc1cnc2cc(OC(F)F)ccc2c1-c1ccc2c(c1)CN(S(N)(=O)=O)CC2] |
| 263 | 6-((6,7-dimethoxyquinolin-4-yl)oxy)-3,4-dihydro-1H-benzo[c][1,2,6]thiadiazine 2,2-dioxide | 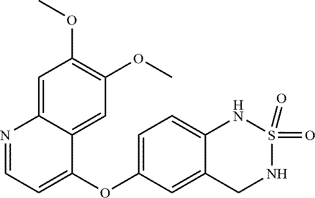 [SMILES: COc1cc2nccc(Oc3ccc4c(c3)CNS(=O)(=O)N4)c2cc1OC] |
| 264 | N-(4-(6-methoxyquinazolin-4-yl)benzyl)sulfamide | 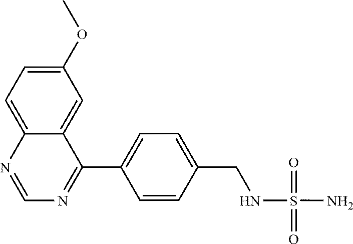 [SMILES: COc1ccc2ncnc(-c3ccc(CNS(N)(=O)=O)cc3)c2c1] |
| 265 | 8-(7-(difluoromethoxy)quinolin-4-yl)-2,8-diazaspiro[4.5]decane-2-sulfonamide | 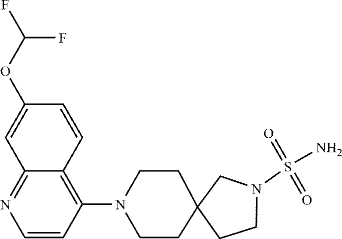 [SMILES: NS(=O)(=O)N1CCC2(CCN(c3ccnc4cc(OC(F)F)ccc34)CC2)C1] |
| 266 | N-(4-(6,7-dimethoxyimidazo[1,2-a]pyridin-3-yl)benzyl)sulfamide | 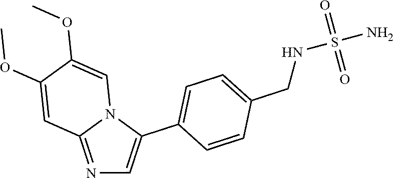 [SMILES: COc1cc2ncc(-c3ccc(CNS(N)(=O)=O)cc3)n2cc1OC] |
| 267 | N-(4-(((7-methoxyquinolin-4-yl)amino)methyl)phenyl)sulfamide | 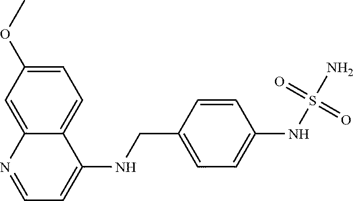 [SMILES: COc1ccc2c(NCc3ccc(NS(N)(=O)=O)cc3)ccnc2c1] |
| 268 | N-(4-((8-fluoro-7-methoxyquinolin-4-yl)oxy)phenyl)sulfamide | 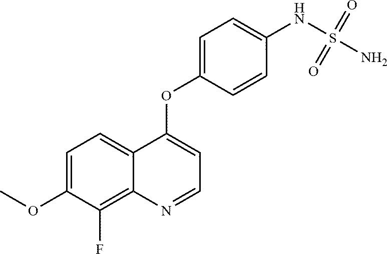 [SMILES: COc1ccc2c(Oc3ccc(NS(N)(=O)=O)cc3)ccnc2c1F] |
| 269 | N-(4-((8-fluoro-7-methoxyquinolin-4-yl)amino)phenyl)sulfamide | 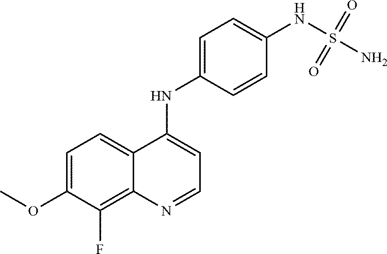 [SMILES: COc1ccc2c(Nc3ccc(NS(N)(=O)=O)cc3)ccnc2c1F] |
| 270 | N-(4-(6,7-dimethoxyquinoxalin-2-yl)phenyl)sulfamide | 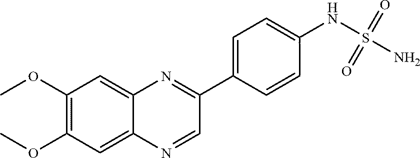 [SMILES: COc1cc2ncc(-c3ccc(NS(N)(=O)=O)cc3)nc2cc1OC] |
| 271 | N-(4-((6,7-dimethoxyquinolin-4-yl)oxy)-2,6-difluorophenyl)sulfamide | 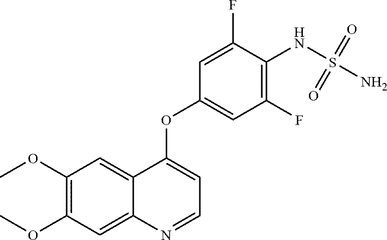 [SMILES: COc1cc2nccc(Oc3cc(F)c(NS(N)(=O)=O)c(F)c3)c2cc1OC] |
| 272 | N-(4-((6,7-dimethoxyquinazolin-4-yl)amino)cyclohexyl)sulfamide | 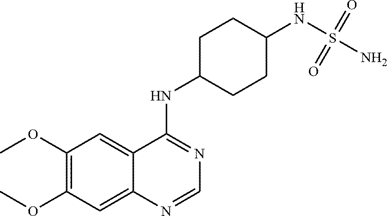 [SMILES: COc1cc2ncnc(NC3CCC(NS(N)(=O)=O)CC3)c2cc1OC] |
| 273 | N-(4-((6,7-dimethoxyquinazolin-4-yl)oxy)phenyl)sulfamide | 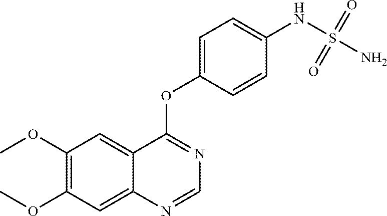 [SMILES: COc1cc2ncnc(Oc3ccc(NS(N)(=O)=O)cc3)c2cc1OC] |
| 274 | 8-(6,7-dimethoxyquinazolin-4-yl)-2,8-diazaspiro[4.5]decane | 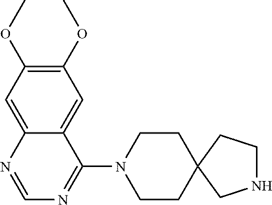 [SMILES: COc1cc2ncnc(N3CCC4(CCNC4)CC3)c2cc1OC] |
| 275 | 7-methoxy-4-(2,8-diazaspiro[4.5]decan-8-yl)quinoline-3-carbonitrile | 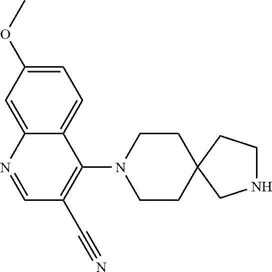 [SMILES: COc1ccc2c(N3CCC4(CCNC4)CC3)c(C#N)cnc2c1] |
| 276 | 7-methoxy-4-(1,2,3,4-tetrahydroisoquinolin-7-yl)quinoline-3-carbonitrile | 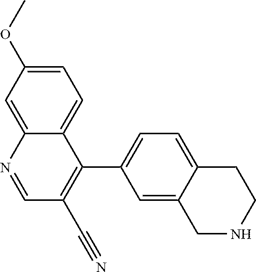 [SMILES: COc1ccc2c(-c3ccc4c(c3)CNCC4)c(C#N)cnc2c1] |
| 277 | 4-(4-(aminomethyl)phenyl)-7-methoxyquinoline-3-carbonitrile | 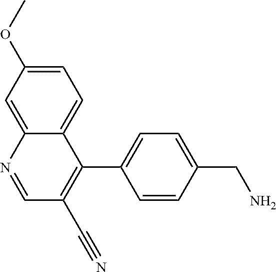 [SMILES: COc1ccc2c(-c3ccc(CN)cc3)c(C#N)cnc2c1] |
| 278 | N-(4-(6,7-dimethoxyquinoxalin-2-yl)benzyl)sulfamide | 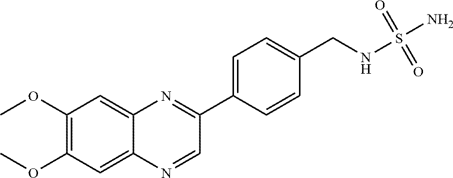 [SMILES: COc1cc2ncc(-c3ccc(CNS(N)(=O)=O)cc3)nc2cc1OC] |
| 279 | N-(4-(1-((7-methoxyquinazolin-4-yl)amino)ethyl)phenyl)sulfamide | 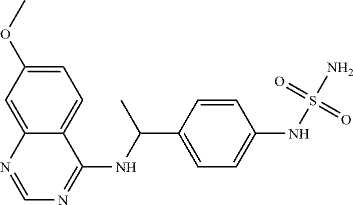 [SMILES: COc1ccc2c(NC(C)c3ccc(NS(N)(=O)=O)cc3)ncnc2c1] |
| 280 | N-(4-((6,7-dimethoxyquinoxalin-2-yl)oxy)phenyl)sulfamide | 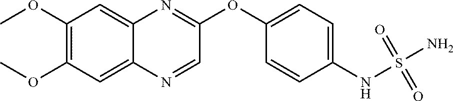 [SMILES: COc1cc2ncc(Oc3ccc(NS(N)(=O)=O)cc3)nc2cc1OC] |
| 281 | N-(1-(6,7-dimethoxyquinolin-4-yl)indolin-5-yl)sulfamide | 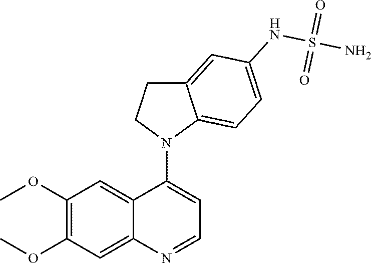 [SMILES: COc1cc2nccc(N3CCc4cc(NS(N)(=O)=O)ccc43)c2cc1OC] |
| 282 | N-(4-((6-fluoro-7-methoxyquinolin-4-yl)oxy)phenyl)sulfamide | 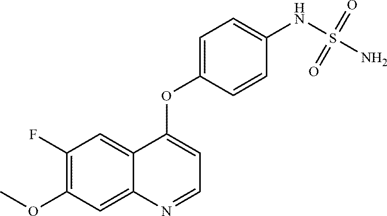 [SMILES: COc1cc2nccc(Oc3ccc(NS(N)(=O)=O)cc3)c2cc1F] |
| 283 | 7-(3-cyclopropyl-6,7-dimethoxyquinolin-4-yl)-3,4-dihydroisoquinoline-2(1H)-sulfonamide | 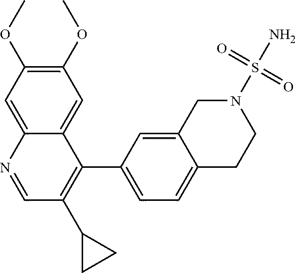 [SMILES: COc1cc2ncc(C3CC3)c(-c3ccc4c(c3)CN(S(N)(=O)=O)CC4)c2cc1OC] |
| 284 | N-(4-(6,7-dimethoxyquinolin-4-yl)benzyl)-N-hydroxyformamide | 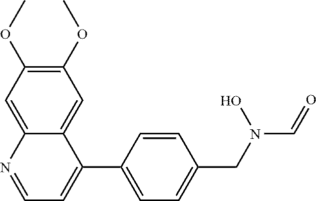 [SMILES: COc1cc2nccc(-c3ccc(CN(O)C=O)cc3)c2cc1OC] |
| 285 | N-cyclopropyl-N-(4-(6,7-dimethoxyquinazolin-4-yl)benzyl)sulfamide | 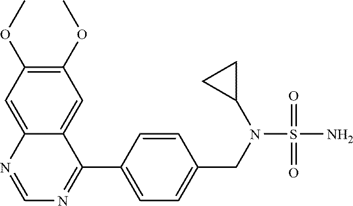 [SMILES: COc1cc2ncnc(-c3ccc(CN(C4CC4)S(N)(=O)=O)cc3)c2cc1OC] |
| 286 | N-(4-((6,7-dimethoxyquinoxalin-2-yl)amino)phenyl)sulfamide | 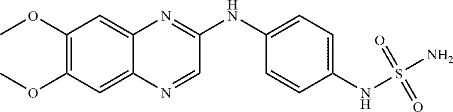 [SMILES: COc1cc2ncc(Nc3ccc(NS(N)(=O)=O)cc3)nc2cc1OC] |
| 287 | N-(4-(2-((6,7-dimethoxyquinazolin-4-yl)amino)propan-2-yl)phenyl)sulfamide | 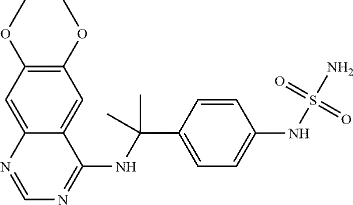 [SMILES: COc1cc2ncnc(NC(C)(C)c3ccc(NS(N)(=O)=O)cc3)c2cc1OC] |
| 288 | 8-(6-fluoroquinazolin-4-yl)-2,8-diazaspiro[4.5]decane-2-sulfonamide | 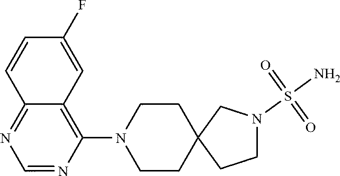 [SMILES: NS(=O)(=O)N1CCC2(CCN(c3ncnc4ccc(F)cc34)CC2)C1] |
| 289 | N-cyclopropyl-N-(4-(7-methoxyquinolin-4-yl)benzyl)sulfamide | 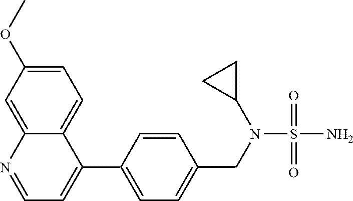 [SMILES: COc1ccc2c(-c3ccc(CN(C4CC4)S(N)(=O)=O)cc3)ccnc2c1] |
| 290 | N-(4-(3-cyano-6,7-dimethoxyquinolin-4-yl)benzyl)-N-cyclopropylsulfamide | 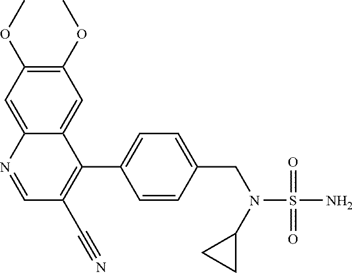 [SMILES: COc1cc2ncc(C#N)c(-c3ccc(CN(C4CC4)S(N)(=O)=O)cc3)c2cc1OC] |
| 291 | N-(6′,7′-dimethoxy-3,4-dihydro-2H-[1,4′-biquinolin]-6-yl)sulfamide | 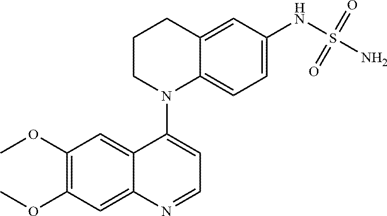 [SMILES: COc1cc2nccc(N3CCCc4cc(NS(N)(=O)=O)ccc43)c2cc1OC] |
| 292 | N-(4-((6,7-dimethoxyquinolin-4-yl)oxy)phenyl)-N-hydroxyformamide | 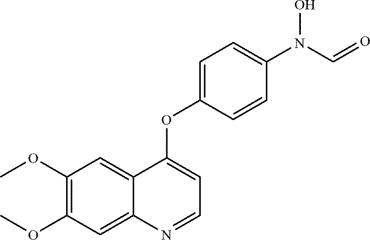 [SMILES: COc1cc2nccc(Oc3ccc(N(O)C=O)cc3)c2cc1OC] |
| 293 | 4-(((7-methoxyquinolin-4-yl)amino)methyl)benzenesulfonam ide | 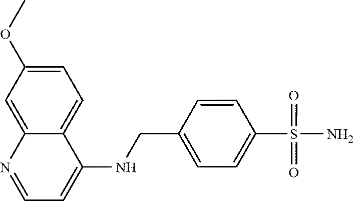 [SMILES: COc1ccc2c(NCc3ccc(S(N)(=O)=O)cc3)ccnc2c1] |
| 294 | N-(4-(3-cyano-6-methoxyquinolin-4-yl)benzyl)-N-cyclopropylsulfamide | 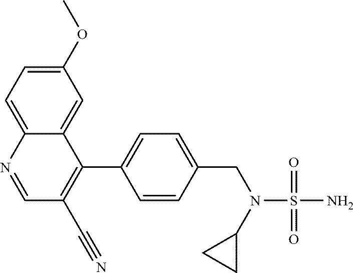 [SMILES: COc1ccc2ncc(C#N)c(-c3ccc(CN(C4CC4)S(N)(=O)=O)cc3)c2c1] |
| 295 | 7-(6,7-dimethoxyquinoxalin-2-yl)-3,4-dihydroisoquinoline-2(1H)-sulfonamide | 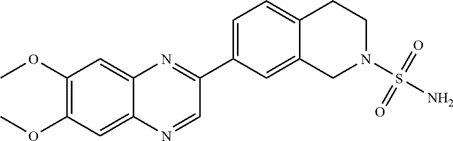 [SMILES: COc1cc2ncc(-c3ccc4c(c3)CN(S(N)(=O)=O)CC4)nc2cc1OC] |
| 296 | N-(4-(7-fluoroquinolin-4-yl)benzyl)sulfamide | 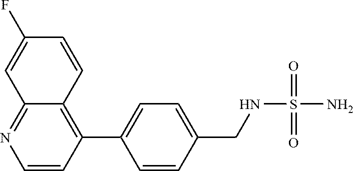 [SMILES: NS(=O)(=O)NCc1ccc(-c2ccnc3cc(F)ccc23)cc1] |
| 297 | N-(2-fluoro-4-((7-methoxyquinolin-4-yl)oxy)phenyl)sulfamide | 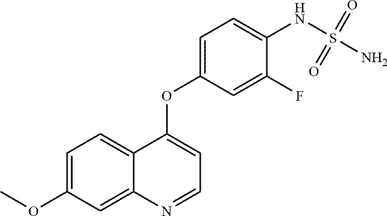 [SMILES: COc1ccc2c(Oc3ccc(NS(N)(=O)=O)c(F)c3)ccnc2c1] |
| 298 | N-(4-((7-fluoroquinolin-4-yl)oxy)phenyl)sulfamide | 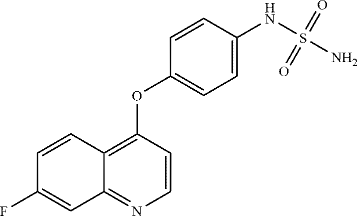 [SMILES: NS(=O)(=O)Nc1ccc(Oc2ccnc3cc(F)ccc23)cc1] |
| 299 | N-(4-(1-((6-methoxyquinazolin-4-yl)amino)ethyl)phenyl)sulfamide | 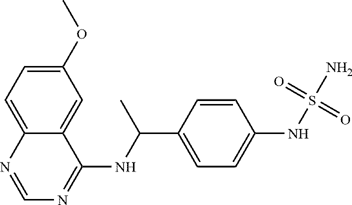 [SMILES: COc1ccc2ncnc(NC(C)c3ccc(NS(N)(=O)=O)cc3)c2c1] |
| 300 | N-(4-((7-methoxy-3-methylquinolin-4-yl)oxy)phenyl)sulfamide | 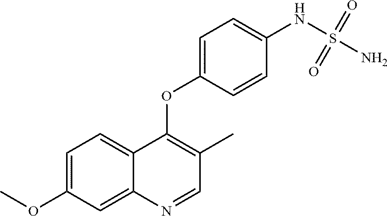 [SMILES: COc1ccc2c(Oc3ccc(NS(N)(=O)=O)cc3)c(C)cnc2c1] |
| 301 | N-(4-(6,7-dimethoxyquinazolin-4-yl)-2-fluorobenzyl)sulfamide | 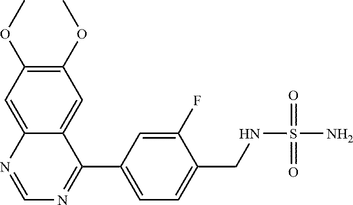 [SMILES: COc1cc2ncnc(-c3ccc(CNS(N)(=O)=O)c(F)c3)c2cc1OC] |
| 302 | N-(4-((7-methoxyquinolin-4-yl)thio)phenyl)sulfamide | 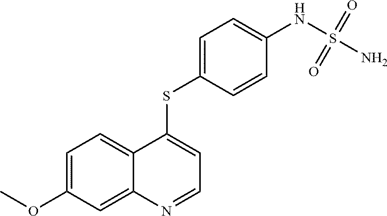 [SMILES: COc1ccc2c(Sc3ccc(NS(N)(=O)=O)cc3)ccnc2c1] |
| 303 | 4-((6,7-dimethoxyquinolin-4-yl)oxy)benzenesulfonamide | 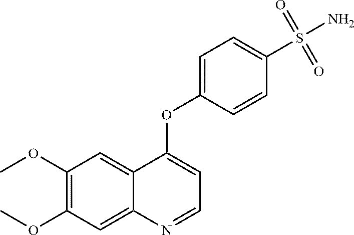 [SMILES: COc1cc2nccc(Oc3ccc(S(N)(=O)=O)cc3)c2cc1OC] |
| 304 | 4-(((7-fluoroquinazolin-4-yl)amino)methyl)benzenesulfonam ide | 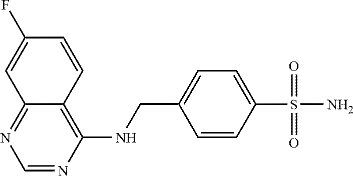 [SMILES: NS(=O)(=O)c1ccc(CNc2ncnc3cc(F)ccc23)cc1] |
| 305 | 7-(7-methoxyquinolin-4-yl)-3,4-dihydroisoquinoline-2(1H)-sulfonamide | 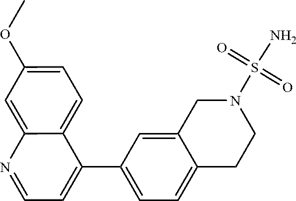 [SMILES: COc1ccc2c(-c3ccc4c(c3)CN(S(N)(=O)=O)CC4)ccnc2c1] |
| 306 | 4-(((6-methoxyquinazolin-4-yl)amino)methyl)benzenesulfonam ide | 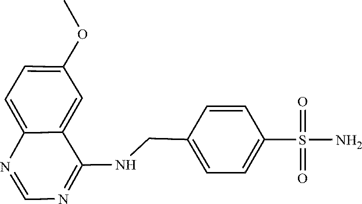 [SMILES: COc1ccc2ncnc(NCc3ccc(S(N)(=O)=O)cc3)c2c1] |
| 307 | N-(4-((6,7-dimethoxyquinolin-4-yl)amino)cyclohexyl)sulfamide | 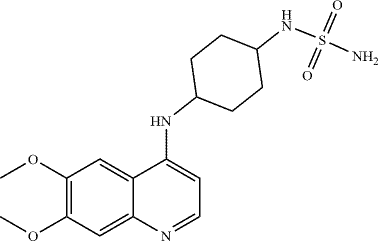 [SMILES: COc1cc2nccc(NC3CCC(NS(N)(=O)=O)CC3)c2cc1OC] |
| 308 | N-(4-((7-methoxyquinolin-4-yl)oxy)-2-methylphenyl)sulfamide | 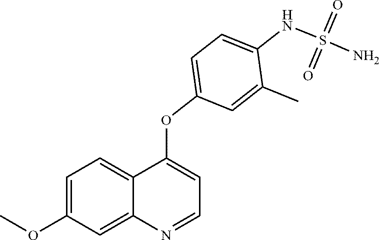 [SMILES: COc1ccc2c(Oc3ccc(NS(N)(=O)=O)c(C)c3)ccnc2c1] |
| 309 | N-benzyl-N-(4-((7-methoxyquinolin-4-yl)oxy)phenyl)sulfamide | 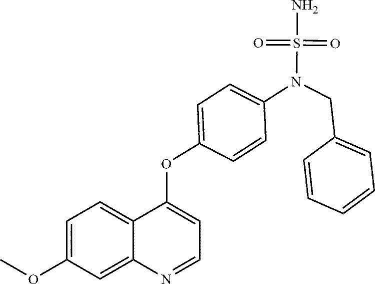 [SMILES: COc1ccc2c(Oc3ccc(N(Cc4ccccc4)S(N)(=O)=O)cc3)ccnc2c1] |
| 310 | N-cyclopropyl-N-(4-((7-methoxyquinolin-4-yl)oxy)phenyl)sulfamide | 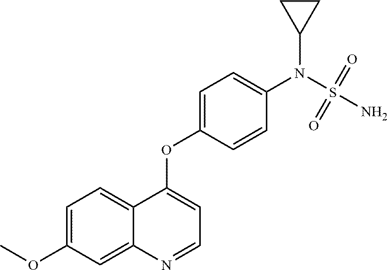 [SMILES: COc1ccc2c(Oc3ccc(N(C4CC4)S(N)(=O)=O)cc3)ccnc2c1] |
| 311 | 7-(7-fluoroquinolin-4-yl)-3,4-dihydroisoquinoline-2(1H)-sulfonamide | 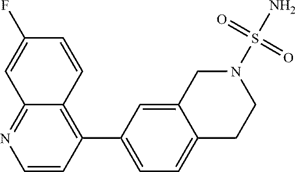 [SMILES: NS(=O)(=O)N1CCc2ccc(-c3ccnc4cc(F)ccc34)cc2C1] |
| 312 | (4-((7-methoxyquinolin-4-yl)oxy)phenyl)methanesulfonamid e | 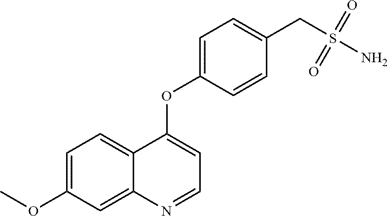 [SMILES: COc1ccc2c(Oc3ccc(CS(N)(=O)=O)cc3)ccnc2c1] |
| 313 | N-(1-(6,7-dimethoxyquinolin-4-yl)-1H-indol-5-yl)sulfamide | 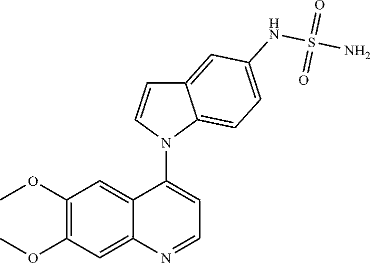 [SMILES: COc1cc2nccc(-n3ccc4cc(NS(N)(=O)=O)ccc43)c2cc1OC] |
| 314 | N-((5-(6,7-dimethoxyquinolin-4-yl)pyridin-2-yl)methyl)sulfamide | 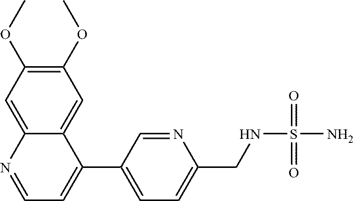 [SMILES: COc1cc2nccc(-c3ccc(CNS(N)(=O)=O)nc3)c2cc1OC] |
| 315 | N-(2-cyano-4-((7-methoxyquinolin-4-yl)oxy)phenyl)sulfamide | 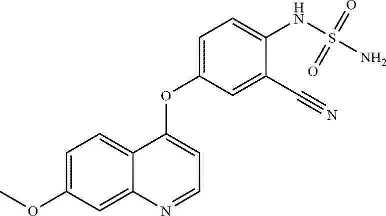 [SMILES: COc1ccc2c(Oc3ccc(NS(N)(=O)=O)c(C#N)c3)ccnc2c1] |
| 316 | 7-(3-cyano-6-fluoro-7-methoxyquinolin-4-yl)-3,4-dihydroisoquinoline-2(1H)-sulfonamide | 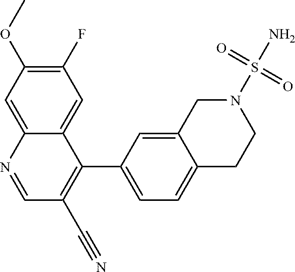 [SMILES: COc1cc2ncc(C#N)c(-c3ccc4c(c3)CN(S(N)(=O)=O)CC4)c2cc1F] |
| 317 | N-(4-((7-methoxyquinolin-4-yl)oxy)-3-(trifluoromethyl)phenyl)sulfamide | 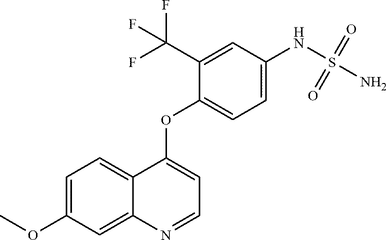 [SMILES: COc1ccc2c(Oc3ccc(NS(N)(=O)=O)cc3C(F)(F)F)ccnc2c1] |
| 318 | 6-((7-methoxyquinolin-4-yl)oxy)-3,4-dihydroquinolin-2(1H)-one | 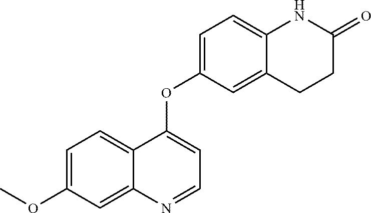 [SMILES: COc1ccc2c(Oc3ccc4c(c3)CCC(=O)N4)ccnc2c1] |
| 319 | 6-((7-methoxyquinolin-4-yl)oxy)-3,4-dihydroquinoline-1(2H)-sulfonamide | 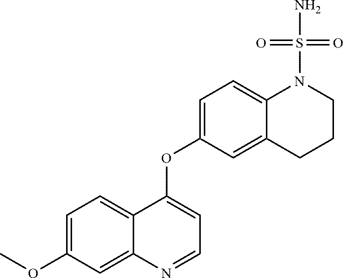 [SMILES: COc1ccc2c(Oc3ccc4c(c3)CCCN4S(N)(=O)=O)ccnc2c1] |
| 320 | N-(6-((7-methoxyquinolin-4-yl)oxy)pyridin-3-yl)sulfamide | 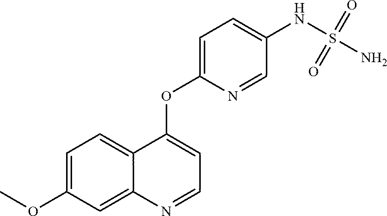 [SMILES: COc1ccc2c(Oc3ccc(NS(N)(=O)=O)cn3)ccnc2c1] |
| 321 | N-(4-((1H-pyrrolo[2,3-b]pyridin-4-yl)oxy)phenyl)sulfamide | 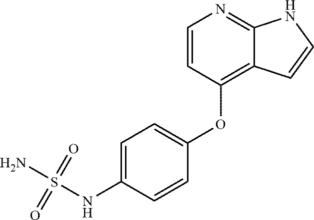 [SMILES: NS(=O)(=O)Nc1ccc(Oc2ccnc3[nH]ccc23)cc1] |
| 322 | N-(4-((7-methoxyquinazolin-4-yl)oxy)phenyl)sulfamide | 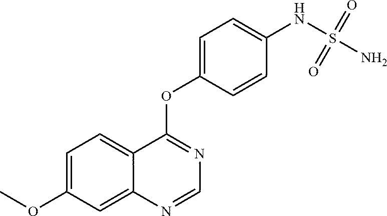 [SMILES: COc1ccc2c(Oc3ccc(NS(N)(=O)=O)cc3)ncnc2c1] |
| 323 | N-(3-methoxy-4-((7-methoxyquinolin-4-yl)oxy)phenyl)sulfamide | 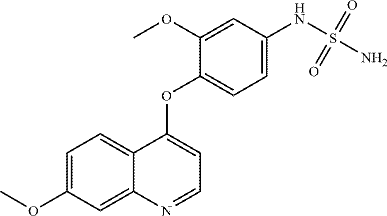 [SMILES: COc1ccc2c(Oc3ccc(NS(N)(=O)=O)cc3OC)ccnc2c1] |
| 324 | N-cyclopropyl-N-(4-(7-methoxyquinazolin-4-yl)benzyl)sulfamide | 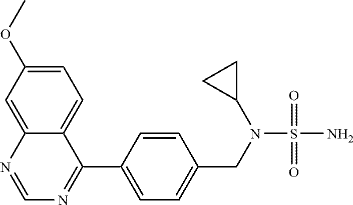 [SMILES: COc1ccc2c(-c3ccc(CN(C4CC4)S(N)(=O)=O)cc3)ncnc2c1] |
| 325 | 5-(((6,7-dimethoxyquinazolin-4-yl)amino)methyl)benzo[c][1,2]oxa borol-1(3H)-ol | 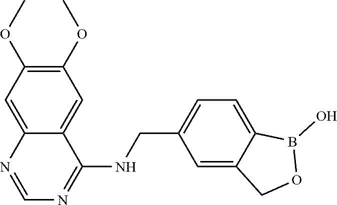 [SMILES: COc1cc2ncnc(NCc3ccc4c(c3)COB4O)c2cc1OC] |
| 326 | N-(4-((7-methoxyquinolin-4-yl)oxy)-3-methylphenyl)sulfamide | 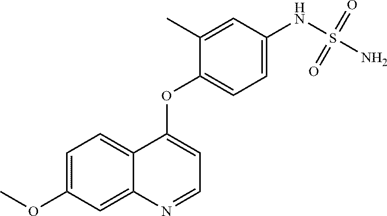 [SMILES: COc1ccc2c(Oc3ccc(NS(N)(=O)=O)cc3C)ccnc2c1] |
| 327 | 4-((3-cyano-7-methoxyquinolin-4-yl)oxy)benzenesulfonamide | 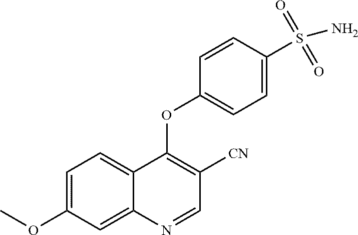 [SMILES: COc1ccc2c(Oc3ccc(S(N)(=O)=O)cc3)c(C#N)cnc2c1] |
| 328 | N-(4-(7-methoxyquinolin-4-yl)-3-methylbenzyl)sulfamide | 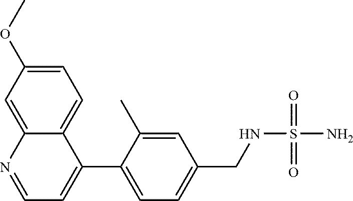 [SMILES: COc1ccc2c(-c3ccc(CNS(N)(=O)=O)cc3C)ccnc2c1] |
| 329 | N-(3-chloro-4-((7-methoxyquinolin-4-yl)oxy)phenyl)sulfamide | 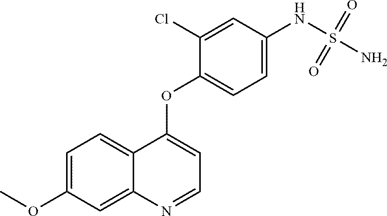 [SMILES: COc1ccc2c(Oc3ccc(NS(N)(=O)=O)cc3Cl)ccnc2c1] |
| 330 | N-(4-((7-methoxy-1,6-naphthyridin-4-yl)oxy)phenyl)sulfamide | 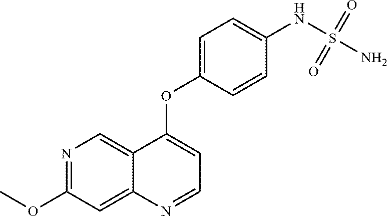 [SMILES: COc1cc2nccc(Oc3ccc(NS(N)(=O)=O)cc3)c2cn1] |
| 331 | N-(2-(1-(6,7-dimethoxyquinazolin-4-yl)piperidin-4-yl)ethyl)sulfamide | 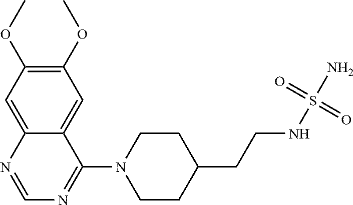 [SMILES: COc1cc2ncnc(N3CCC(CCNS(N)(=O)=O)CC3)c2cc1OC] |
| 332 | 2-(4-((6,7-dimethoxyquinazolin-4-yl)amino)phenyl)acetic acid | 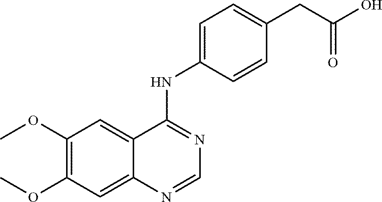 [SMILES: COc1cc2ncnc(Nc3ccc(CC(=O)O)cc3)c2cc1OC] |
| 333 | 4-(6,7-dimethoxyquinazolin-4-yl)benzenesulfonamide | 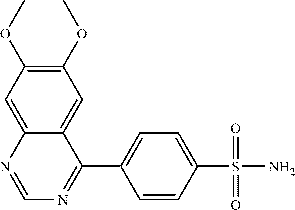 [SMILES: COc1cc2ncnc(-c3ccc(S(N)(=O)=O)cc3)c2cc1OC] |
| 334 | 2-(1-(6,7-dimethoxyquinazolin-4-yl)piperidin-4-yl)ethan-1-amine | 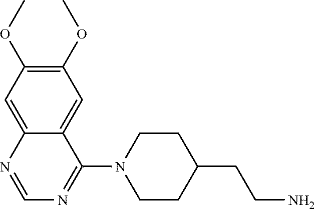 [SMILES: COc1cc2ncnc(N3CCC(CCN)CC3)c2cc1OC] |
| 335 | N-(2-(1-(6,7-dimethoxyquinazolin-4-yl)piperidin-4-yl)ethyl)acetamide | 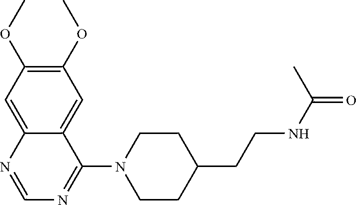 [SMILES: COc1cc2ncnc(N3CCC(CCNC(C)=O)CC3)c2cc1OC] |
| 336 | N-(benzo[d][1,3]dioxol-5-ylmethyl)-6,7-dimethoxyquinazolin-4-amine | 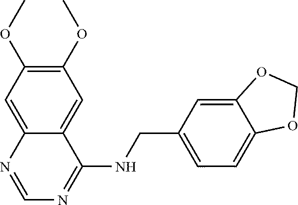 [SMILES: COc1cc2ncnc(NCc3ccc4c(c3)OCO4)c2cc1OC] |
| 337 | 4-(2-ethoxy-5-((4-methylpiperazin-1-yl)sulfonyl)phenyl)-6,7-dimethoxyquinazoline | 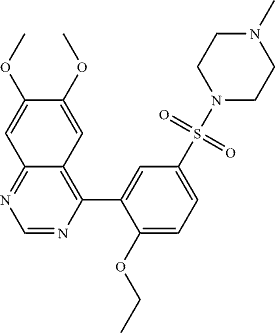 [SMILES: CCOc1ccc(S(=O)(=O)N2CCN(C)CC2)cc1-c1ncnc2cc(OC)c(OC)cc12] |
| 338 | N-(2-(1-(6,7-dimethoxyquinazolin-4-yl)piperidin-4-yl)ethyl)methanesulfonamide | 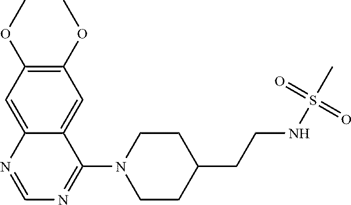 [SMILES: COc1cc2ncnc(N3CCC(CCNS(C)(=O)=O)CC3)c2cc1OC] |
| 339 | N-((1-(6,7-dimethoxyquinazolin-4-yl)piperidin-4-yl)methyl)sulfamide | 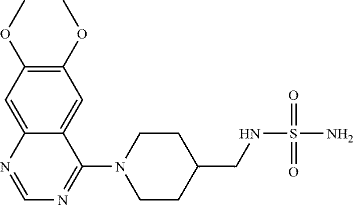 [SMILES: COc1cc2ncnc(N3CCC(CNS(N)(=O)=O)CC3)c2cc1OC] |
| 340 | 2-(4-((3,4-dimethoxyphenyl)sulfonyl)piperazi n-1-yl)ethan-1-ol | 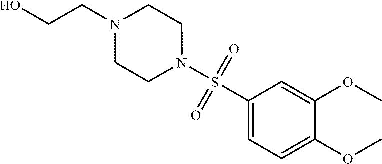 [SMILES: COc1ccc(S(=O)(=O)N2CCN(CCO)CC2)cc1OC] |
| 341 | 4-((6,7-dimethoxyquinolin-4-yl)oxy)aniline | 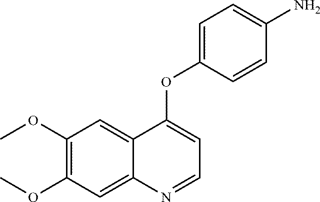 [SMILES: COc1cc2nccc(Oc3ccc(N)cc3)c2cc1OC] |
| 342 | 4-((7-methoxyquinolin-4-yl)oxy)aniline | 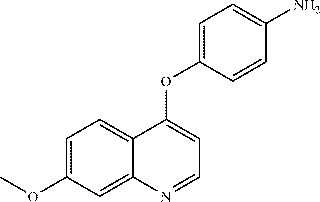 [SMILES: COc1ccc2c(Oc3ccc(N)cc3)ccnc2c1] |
| 343 | N1-(6,7-dimethoxyquinazolin-4-yl)benzene-1,4-diamine | 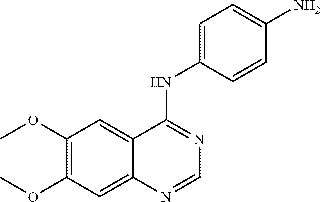 [SMILES: COc1cc2ncnc(Nc3ccc(N)cc3)c2cc1OC] |
| 344 | 4-((6,7-dimethoxyquinolin-4-yl)oxy)-2-fluoroaniline | 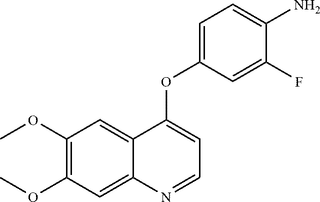 [SMILES: COc1cc2nccc(Oc3ccc(N)c(F)c3)c2cc1OC] |
| 345 | N-(4-(7-methoxy quinolin-4-yl)-3-methylbenzyl)sulfamide | 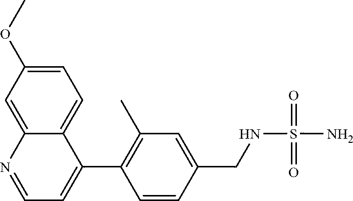 [SMILES: COc1ccc2c(-c3ccc(CNS(N)(=O)=O)cc3C)ccnc2c1] |
| 346 | N-(4-((6-methoxy-1,5-naphthyridin-4-yl)oxy)phenyl)sulfamide | 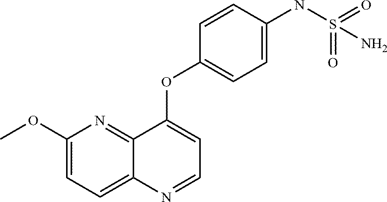 [SMILES: COc1ccc2nccc(Oc3ccc(NS(N)(=O)=O)cc3)c2n1] |
| 347 | 2-methoxy-4-((7-methoxyquinolin-4-yl)oxy)benzoic acid | 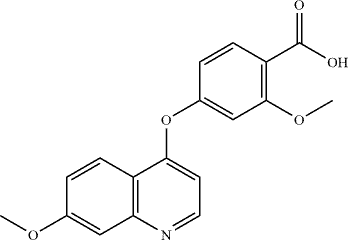 [SMILES: COc1ccc2c(Oc3ccc(C(=O)O)c(OC)c3)ccnc2c1] |
| 348 | N-(4-(6-methoxy benzofuran-3-yl)benzyl) sulfamide | 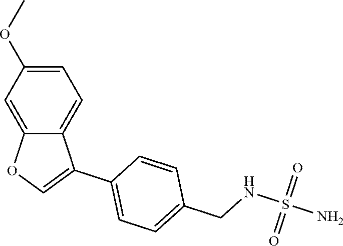 [SMILES: COc1ccc2c(-c3ccc(CNS(N)(=O)=O)cc3)coc2c1] |
| 349 | N-(4-((7-methoxy-1,5-naphthyridin-4-yl)oxy)phenyl) sulfamide | 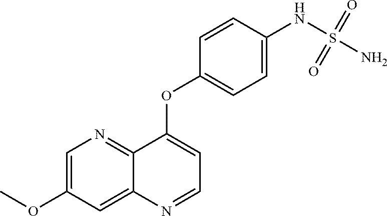 [SMILES: COc1cnc2c(Oc3ccc(NS(N)(=O)=O)cc3)ccnc2c1] |
| 350 | 4-((7-(methoxy-d3)quinolin-4-yl)oxy)benzamide | 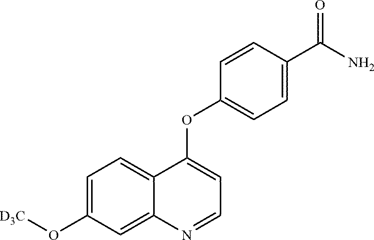 [SMILES: [2H]C([2H])([2H])Oc1ccc2c(Oc3ccc(C(N)=O)cc3)ccnc2c1] |
| 351 | N-(6-((7-methoxy quinolin-4-yl)oxy)-5-methylpyridin-3-yl)sulfamide | 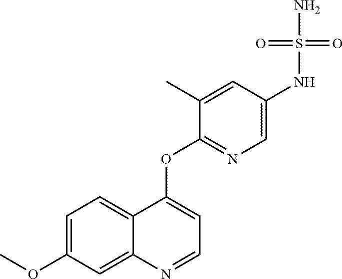 [SMILES: COc1ccc2c(Oc3ncc(NS(N)(=O)=O)cc3C)ccnc2c1] |
| 352 | 4-((7-oxo-7,8-dihydro-1,8-naphthyridin-4-yl)oxy)benzenesulfonamide | 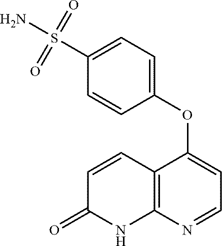 [SMILES: NS(=O)(=O)c1ccc(Oc2ccnc3[nH]c(=O)ccc23)cc1] |
| 353 | 4-((6-methoxy quinolin-4-yl)oxy) benzenesulfamide | 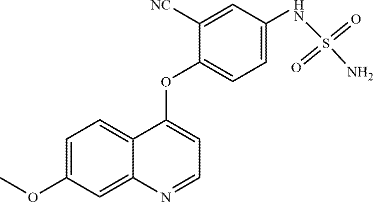 [SMILES: COc1ccc2c(Oc3ccc(NS(N)(=O)=O)cc3C#N)ccnc2c1] |
| 354 | N-((5-(7-methoxyquinolin-4-yl)pyridin-2-yl)methyl)sulfamide | 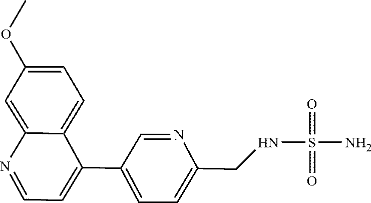 [SMILES: COc1ccc2c(-c3ccc(CNS(N)(=O)=O)nc3)ccnc2c1] |
| 355 | 4-((6-methoxyquinolin-4-yl)oxy)benzenesulfonamide | 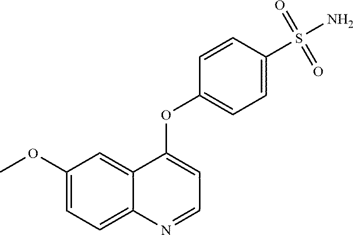 |
| 356 | 4-((7-methoxy quinazolin-4-yl)oxy)benzene sulfonamide | 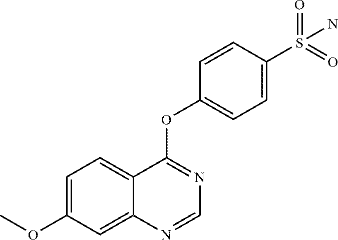 [SMILES: COc1ccc2c(Oc3ccc(S(N)(=O)=O)cc3)ncnc2c1] |
| 357 | N-(2-(4-(7-methoxyquinolin-4-yl)phenyl)propan-2-yl)sulfamide | 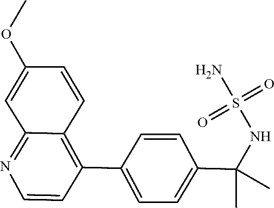 [SMILES: COc1ccc2c(-c3ccc(C(C)(C)NS(N)(=O)=O)cc3)ccnc2c1] |
| 358 | 7-(6-methoxy quinolin-4-yl)-3,4-dihydroisoquinoline-2(1H)-sulfonamide | 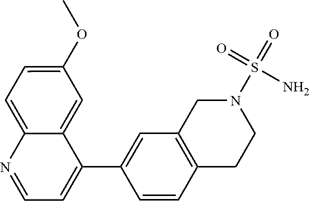 [SMILES: COc1ccc2nccc(-c3ccc4c(c3)CN(S(N)(=O)=O)CC4)c2c1] |
| 359 | 4-((7-methoxy-3-methylquinolin-4-yl)oxy)benzene sulfonamide | 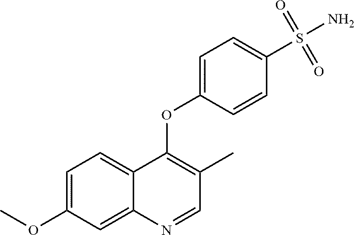 [SMILES: COc1ccc2c(Oc3ccc(S(N)(=O)=O)cc3)c(C)cnc2c1] |
| 360 | N-(3-methoxy-4-((7-(methoxy-d3)quinolin-4-yl)oxy)phenyl)sulfamide | 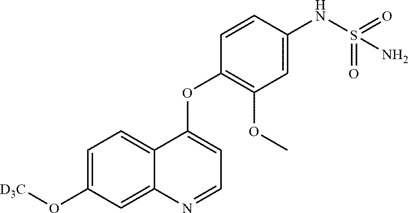 [SMILES: [2H]C([2H])([2H])Oc1ccc2c(Oc3ccc(NS(N)(=O)=O)cc3OC)ccnc2c1] |
| 361 | 4-(7-Methoxy-[1,8]naphthyridin-4-yl-oxy)-benzene sulfonamide | 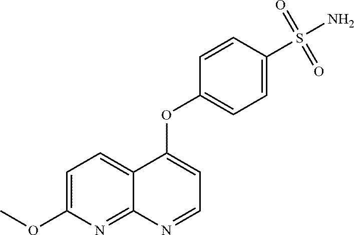 [SMILES: COc1ccc2c(Oc3ccc(S(N)(=O)=O)cc3)ccnc2n1] |
| 362 | N-(4-((7-methoxyquinolin-4-yl)oxy)phenyl)hydrazinesulfonami de | 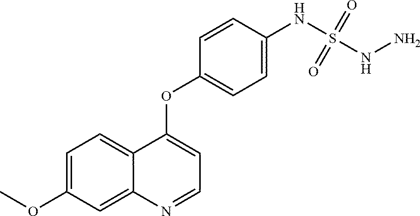 [SMILES: COc1ccc2c(Oc3ccc(NS(=O)(=O)NN)cc3)ccnc2c1] |
| 363 | 8-(7-methoxyquinolin-4-yl)-2,8-diazaspiro[4.5]decane-2-sulfonamide | 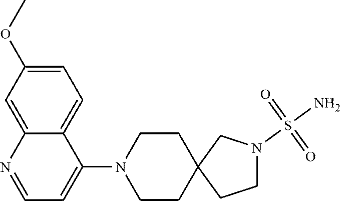 [SMILES: COc1ccc2c(N3CCC4(CC3)CCN(S(N)(=O)=O)C4)ccnc2c1] |
| 364 | 8-(6-methoxyquinolin-4-yl)-2,8-diazaspiro[4.5]decane-2-sulfonamide | 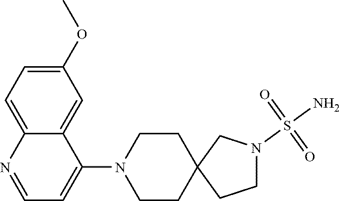 [SMILES: COc1ccc2nccc(N3CCC4(CC3)CCN(S(N)(=O)=O)C4)c2c1] |
| 365 | 7-(7-methoxy-1,8-naphthyridin-4-yl)-3,4-dihydroisoquinoline-2(1H)-sulfonamide | 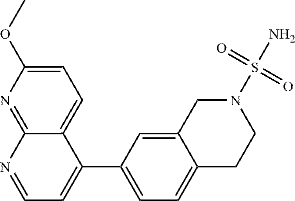 [SMILES: COc1ccc2c(-c3ccc4c(c3)CN(S(N)(=O)=O)CC4)ccnc2n1] |
| 366 | 6-(7-methoxy quinolin-4-yl)-3,4-dihydroisoquinoline-2(1H)-sulfonamide | 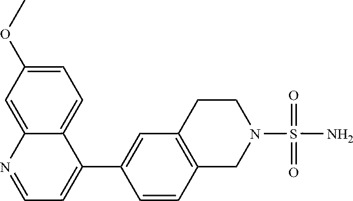 [SMILES: COc1ccc2c(-c3ccc4c(c3)CCN(S(N)(=O)=O)C4)ccnc2c1] |
| 367 | 6-(6,7-dimethoxyquinolin-4-yl)-3,4-dihydroisoquinoline-2(1H)-sulfonamide | 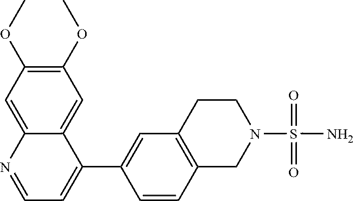 [SMILES: COc1cc2nccc(-c3ccc4c(c3)CCN(S(N)(=O)=O)C4)c2cc1OC] |
| 368 | N-(4-(7-methoxyquinolin-4-yl)benzyl)-N-methyl sulfamide | 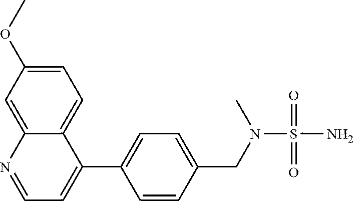 [SMILES: COc1ccc2c(-c3ccc(CN(C)S(N)(=O)=O)cc3)ccnc2c1] |
| 369 | N-(4-(7-methoxy-1,5-naphthyridin-4-yl)benzyl)sulfamide | 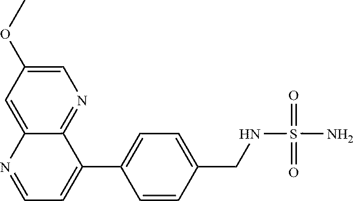 [SMILES: COc1cnc2c(-c3ccc(CNS(N)(=O)=O)cc3)ccnc2c1] |
| 370 | N-(4-(7-(methoxy-d3)quinolin-4-yl)phenyl)sulfamide | 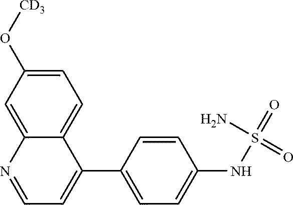 [SMILES: [2H]C([2H])([2H])Oc1ccc2c(-c3ccc(NS(N)(=O)=O)cc3)ccnc2c1] |
| 371 | 7-(7-methoxy-3-methylquinolin-4-yl)-3,4-dihydroisoquinoline-2(1H)-sulfonamide | 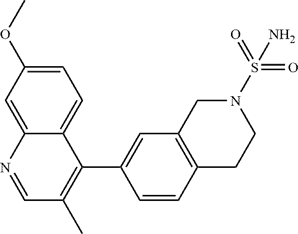 [SMILES: COc1ccc2c(-c3ccc4c(c3)CN(S(N)(=O)=O)CC4)c(C)cnc2c1] |
| 372 | N-(3-(6-methoxy-1H-indol-3-yl)benzyl)sulfamide | 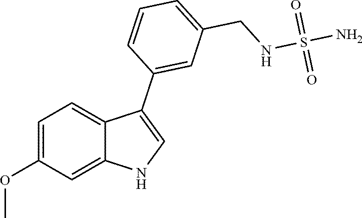 [SMILES: COc1ccc2c(-c3cccc(CNS(N)(=O)=O)c3)c[nH]c2c1] |
| 373 | N-(2-methoxy-4-(7-methoxyquinolin-4-yl)benzyl)sulfamide | 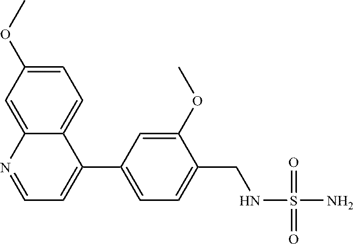 [SMILES: COc1ccc2c(-c3ccc(CNS(N)(=O)=O)c(OC)c3)ccnc2c1] |
| 374 | N-(4-(7-methoxy quinolin-4-yl)-2-methylbenzyl) sulfamide | 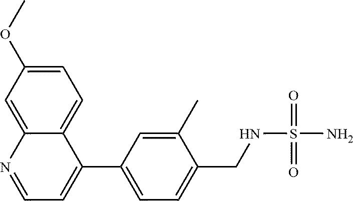 [SMILES: COc1ccc2c(-c3ccc(CNS(N)(=O)=O)c(C)c3)ccnc2c1] |
| 375 | N-(7-fluoro-1-(7-methoxyquinolin-4-yl)indolin-5-yl)sulfamide | 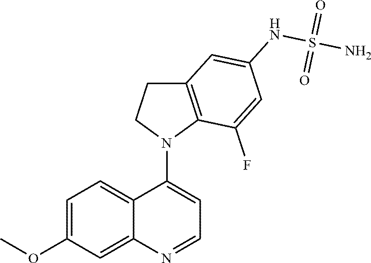 [SMILES: COc1ccc2c(N3CCc4cc(NS(N)(=O)=O)cc(F)c43)ccnc2c1] |
| 376 | N-(1-(8-fluoro-7-methoxyquinolin-4-yl)indolin-5-yl)sulfamide | 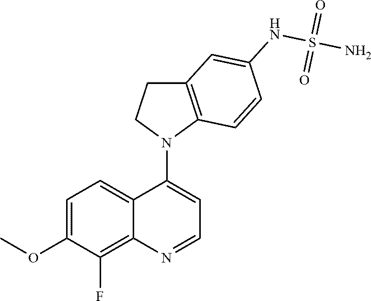 [SMILES: COc1ccc2c(N3CCc4cc(NS(N)(=O)=O)ccc43)ccnc2c1F] |
| 377 | 4-((7-methoxy-3-(trifluoromethyl) quinolin-4-yl)oxy) benzenesulfonamide | 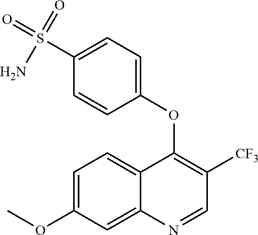 [SMILES: COc1ccc2c(Oc3ccc(S(N)(=O)=O)cc3)c(C(F)(F)F)cnc2c1] |
| 378 | N-(1-(7-methoxy-3-methylquinolin-4-yl)indolin-5-yl)sulfamide | 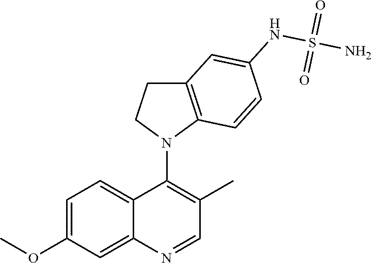 [SMILES: COc1ccc2c(N3CCc4cc(NS(N)(=O)=O)ccc43)c(C)cnc2c1] |
| 379 | N-(4-fluoro-1-(7-methoxyquinolin-4-yl)indolin-5-yl)sulfamide | 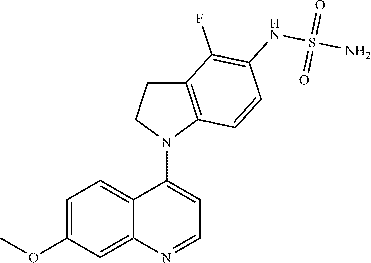 [SMILES: COc1ccc2c(N3CCc4c3ccc(NS(N)(=O)=O)c4F)ccnc2c1] |
| 380 | N-(3-chloro-4-((7-(methoxy-d3)quinolin-4-yl)oxy)phenyl)sulfamide | 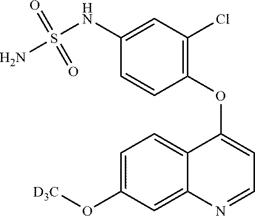 [SMILES: [2H]C([2H])([2H])Oc1ccc2c(Oc3ccc(NS(N)(=O)=O)cc3Cl)ccnc2c1] |
| 381 | N-(4-(7-methoxy-3-Fluoro-quinolin-4-yl)benzyl) Sulfamide | 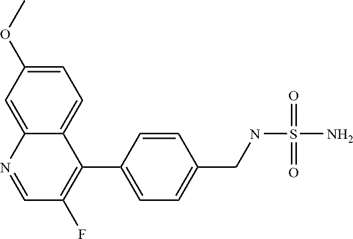 [SMILES: COc1ccc2c(-c3ccc(CNS(N)(=O)=O)cc3)c(F)cnc2c1] |
| 382 | 3-fluoro-4-((7-methoxyquinolin-4-yl)oxy)benzene sulfonamide | 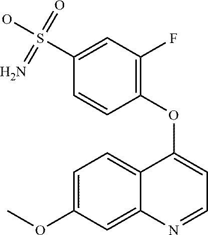 [SMILES: COc1ccc2c(Oc3ccc(S(=N)(=O)O)cc3F)ccnc2c1] |
| 383 | N-(3-methoxy-4-(7-methoxyquinolin-4-yl)benzyl)sulfamide | 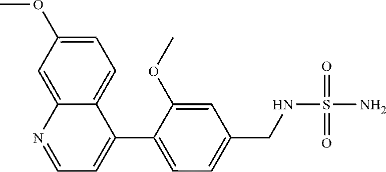 [SMILES: COc1ccc2c(-c3ccc(CNS(N)(=O)=O)cc3OC)ccnc2c1] |
| 384 | N-(4-(7-(methoxy-d3)quinolin-4-yl)benzyl)sulfamide | 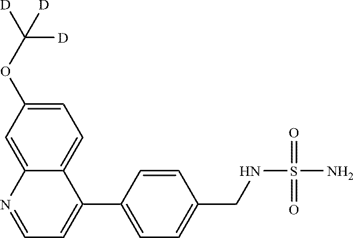 [SMILES: [2H]C([2H])([2H])Oc1ccc2c(-c3ccc(CNS(N)(=O)=O)cc3)ccnc2c1] |
| 385 | N-((1-(7-methoxyquinolin-4-yl)-1H-imidazol-4-yl)methyl)sulfamide | 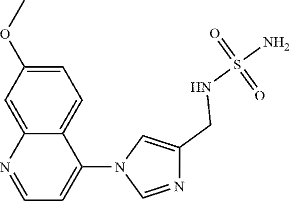 [SMILES: COc1ccc2c(-n3cnc(CNS(N)(=O)=O)c3)ccnc2c1] |
| 386 | N-(4-(7-methoxy-3-(trifluoromethyl) quinolin-4-yl)benzyl) sulfamide | 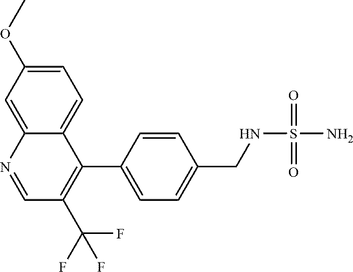 [SMILES: COc1ccc2c(-c3ccc(CNS(N)(=O)=O)cc3)c(C(F)(F)F)cnc2c1] |
| 387 | ((6-(7-methoxy quinolin-4-yl)pyridin-3-yl)methyl)sulfamide | 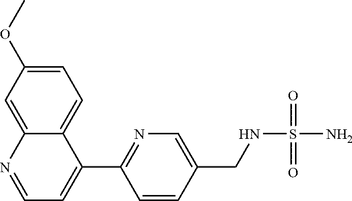 [SMILES: COc1ccc2c(-c3ccc(CNS(N)(=O)=O)cn3)ccnc2c1] |
| 388 | 4-((7-(methoxy-d3)quinolin-4-yl)oxy)benzene sulfonamide | 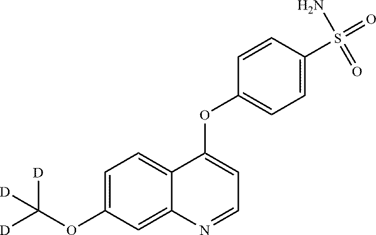 [SMILES: [2H]C([2H])([2H])Oc1ccc2c(Oc3ccc(S(N)(=O)=O)cc3)ccnc2c1] |
| 389 | N-((5-(7-methoxy quinolin-4-yl)thiophen-2-yl)methyl)sulfamide | 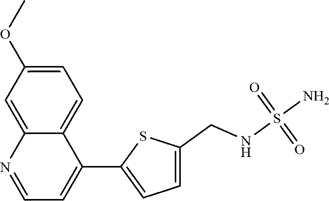 [SMILES: COc1ccc2c(-c3ccc(CNS(N)(=O)=O)s3)ccnc2c1] |
| 390 | N-(2-methoxy-4-(7-(methoxy-d3)quinolin-4-yl)benzyl)sulfamide | 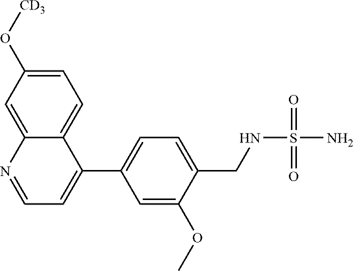 [SMILES: [2H]C([2H])([2H])Oc1ccc2c(-c3ccc(CNS(N)(=O)=O)c(OC)c3)ccnc2c1] |
| 391 | N-(1-(7-methoxyquinolin-4-yl)indolin-6-yl)sulfamide | 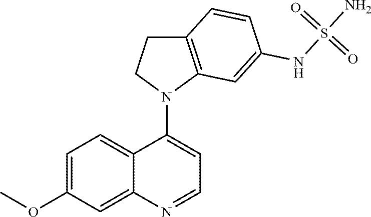 [SMILES: COc1ccc2c(N3CCc4ccc(NS(N)(=O)=O)cc43)ccnc2c1] |
| 392 | N-(1-(7-methoxyquinazolin-4-yl)indolin-5-yl)sulfamide | 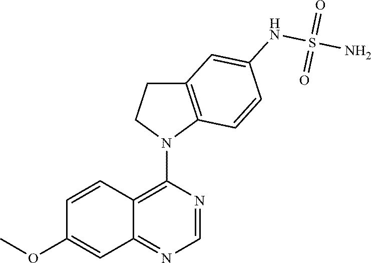 [SMILES: COc1ccc2c(N3CCc4cc(NS(N)(=O)=O)ccc43)ncnc2c1] |
| 393 | 4-((7-methoxy-1,6-naphthyridin-4-yl)oxy)benzenesulfonamide | 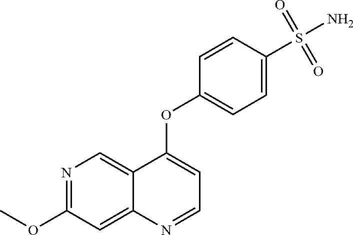 [SMILES: COc1cc2nccc(Oc3ccc(S(N)(=O)=O)cc3)c2cn1] |
| 394 | N-(4-(5-fluoro-7-methoxyquinolin-4-yl)benzyl)sulfamide | 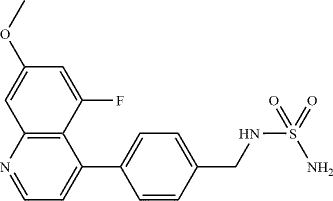 [SMILES: COc1cc(F)c2c(-c3ccc(CNS(N)(=O)=O)cc3)ccnc2c1] |
| 395 | N-(4-(3-cyano-6-fluoro-7-methoxyquinolin-4-yl)benzyl)sulfamide | 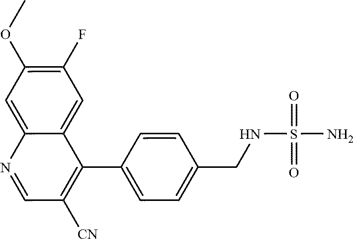 [SMILES: COc1cc2ncc(C#N)c(-c3ccc(CNS(N)(=O)=O)cc3)c2cc1F] |
| 396 | N-(4-(7-methoxyquinolin-4-yl)phenyl)sulfamide | 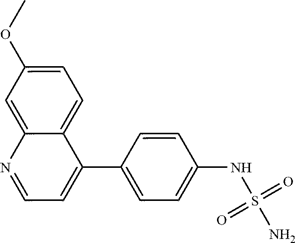 [SMILES: COc1ccc2c(-c3ccc(NS(N)(=O)=O)cc3)ccnc2c1] |
| 397 | N-(4-((7-methoxy-2-methylquinolin-4-yl)oxy)phenyl)sulfamide | 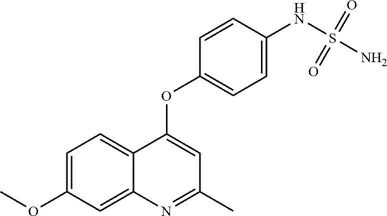 [SMILES: COc1ccc2c(Oc3ccc(NS(N)(=O)=O)cc3)cc(C)nc2c1] |
| 398 | N-((4-(7-methoxyquinolin-4-yl)phenyl)methyl-d₂)sulfamide | 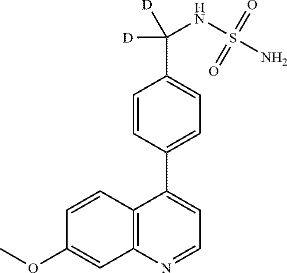 [SMILES: [2H]C([2H])(NS(N)(=O)=O)c1ccc(-c2ccnc3cc(OC)ccc23)cc1] |
| 399 | (4-((7-methoxychroman-4-yl)oxy)phenyl)sulfamide | 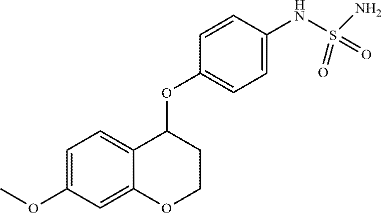 [SMILES: COc1ccc2c(c1)OCCC2Oc1ccc(NS(N)(=O)=O)cc1] |
| 400 | 4-((7-methoxy-4-oxoquinazolin-3(4H)-yl)methyl)benzenesulfonamide | 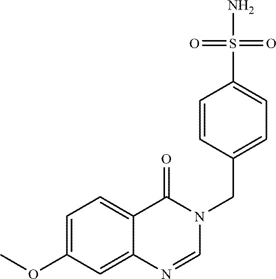 [SMILES: COc1ccc2c(=O)n(Cc3ccc(S(N)(=O)=O)cc3)cnc2c1] |
| 401 | N-(4-((7-methoxy-3-oxo-2,3-dihydro-4H-benzo[b][1,4]oxazin-4-yl)methyl)phenyl)sulfamide | 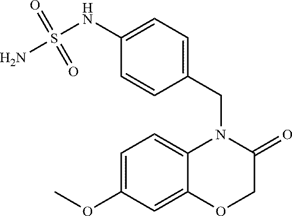 [SMILES: COc1ccc2c(c1)OCC(=O)N2Cc1ccc(NS(N)(=O)=O)cc1] |
| 402 | 5-(7-methoxyquinolin-4-yl)isoindoline-2-sulfonamide | 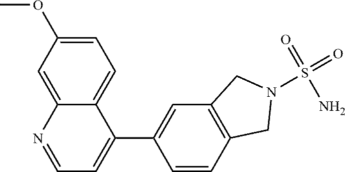 [SMILES: COc1ccc2c(-c3ccc4c(c3)CN(S(N)(=O)=O)C4)ccnc2c1] |
| 403 | N-(4-(7-methoxyquinolin-4-yl)phenethyl)sulfamide | 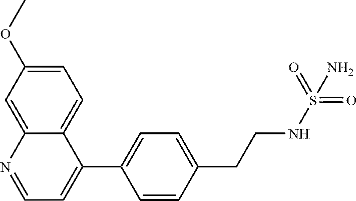 [SMILES: COc1ccc2c(-c3ccc(CCNS(N)(=O)=O)cc3)ccnc2c1] |
| 404 | 5-((7-methoxy quinolin-4-yl)oxy)indoline-1-sulfonamide | 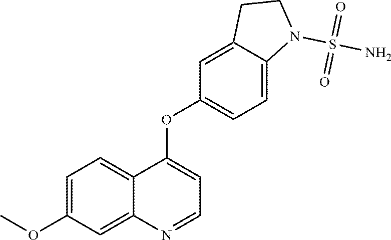 [SMILES: COc1ccc2c(Oc3ccc4c(c3)CCN4S(N)(=O)=O)ccnc2c1] |
| 405 | N-(4-(7-methoxy-2,3-dihydro-4H-benzo[b][1,4]oxazin-4-yl)benzyl)sulfamide | 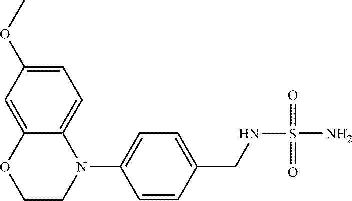 [SMILES: COc1ccc2c(c1)OCCN2c1ccc(CNS(N)(=O)=O)cc1] |
| 406 | (4-(7-methoxy quinolin-4-yl) phenyl)methane sulfonamide | 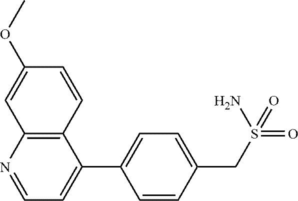 [SMILES: COc1ccc2c(-c3ccc(CS(N)(=O)=O)cc3)ccnc2c1] |
| 407 | N-(4-((7-methoxy-3-(trifluoromethyl)quinolin-4-yl)oxy)phenyl)sulfamide | 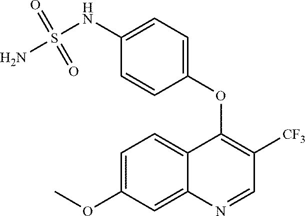 [SMILES: COc1ccc2c(Oc3ccc(NS(N)(=O)=O)cc3)c(C(F)(F)F)cnc2c1] |
| 408 | 7-(7-methoxy-3-(trifluoromethyl)quinolin-4-yl)-3,4-dihydroisoquinoline-2(1H)-sulfonamide | 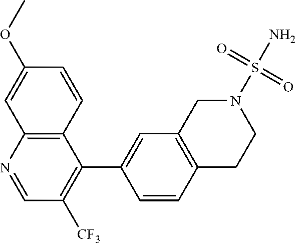 [SMILES: COc1ccc2c(-c3ccc4c(c3)CN(S(N)(=O)=O)CC4)c(C(F)(F)F)cnc2c1] |
| 409 | 6-fluoro-7-(7-methoxyquinolin-4-yl)-3,4-dihydroisoquinoline-2(1H)-sulfamide | 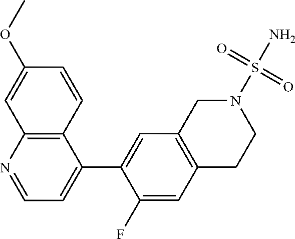 [SMILES: COc1ccc2c(-c3cc4c(cc3F)CCN(S(N)(=O)=O)C4)ccnc2c1] |
| 410 | 8-fluoro-7-(7-methoxyquinolin-4-yl)-3,4-dihydroisoquinoline-2(1H)-sulfamide | 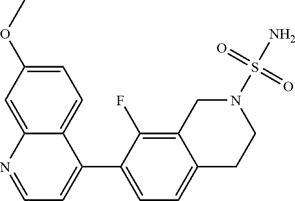 [SMILES: COc1ccc2c(-c3ccc4c(c3F)CN(S(N)(=O)=O)CC4)ccnc2c1] |
| 411 | N-(4-((6-methoxy-1,7-naphthyridin-4-yl)oxy)phenyl)sulfamide | 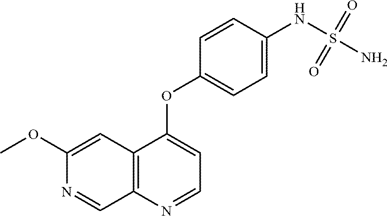 [SMILES: COc1cc2c(Oc3ccc(NS(N)(=O)=O)cc3)ccnc2cn1] |
| 412 | N-(1-(5-fluoro-7-methoxyquinolin-4-yl)indolin-5 -yl)sulfamide | 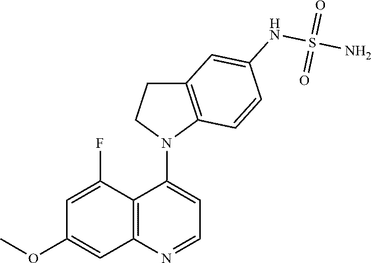 [SMILES: COc1cc(F)c2c(N3CCc4cc(NS(N)(=O)=O)ccc43)ccnc2c1] |
| 413 | N-(3-chloro-1-(7-methoxyquinolin-4-yl)-1H-indol-5-yl)sulfamide | 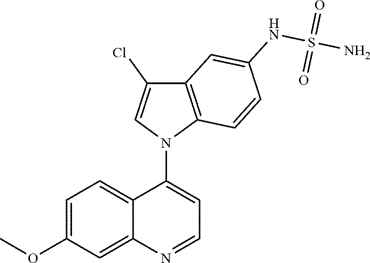 [SMILES: COc1ccc2c(-n3cc(Cl)c4cc(NS(N)(=O)=O)ccc43)ccnc2c1] |
| 414 | N-((1-(7-methoxyquinolin-4-yl)-1H-pyrazol-4-yl)methyl)sulfamide | 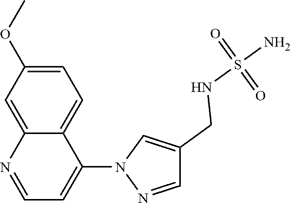 [SMILES: COc1ccc2c(-n3cc(CNS(N)(=O)=O)cn3)ccnc2c1] |
| 415 | N-(4-(7-methoxy-1,6-naphthyridin-4-yl)benzyl)sulfamide | 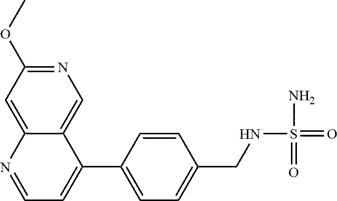 [SMILES: COc1cc2nccc(-c3ccc(CNS(N)(=O)=O)cc3)c2cn1] |
| 416 | N-(1-(4-(7-methoxyquinolin-4-yl)phenyl)ethyl)sulfamide | 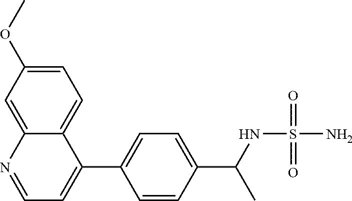 [SMILES: COc1ccc2c(-c3ccc(C(C)NS(N)(=O)=O)cc3)ccnc2c1] |
| 417 | N-(4-(((7-methoxy-1,8-naphthyridin-4-yl)amino)methyl)phenyl)sulfamide | 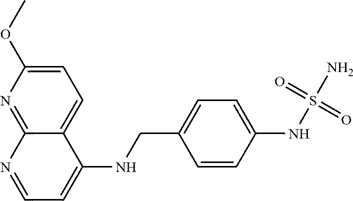 [SMILES: COc1ccc2c(NCc3ccc(NS(N)(=O)=O)cc3)ccnc2n1] |
| 418 | N-(1-(7-methoxyquinolin-4-yl)indolin-5-yl)-N-methylsulfamide | 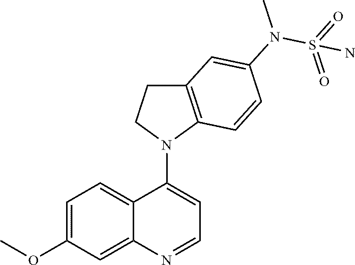 [SMILES: COc1ccc2c(N3CCc4cc(N(C)S(N)(=O)=O)ccc43)ccnc2c1] |
| 419 | N-(3-chloro-4-(7-methoxyquinolin-4-yl)benzyl)sulfamide | 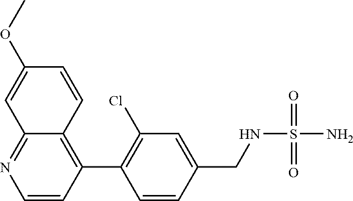 [SMILES: COc1ccc2c(-c3ccc(CNS(N)(=O)=O)cc3Cl)ccnc2c1] |
| 420 | 8-(7-(methoxy-d3)quinolin-4-yl)-2,8-diazaspiro[4.5]decane-2-sulfonamide | 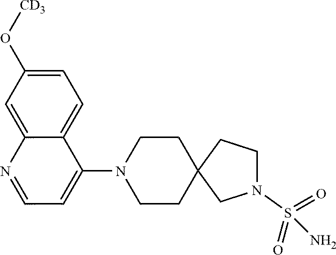 [SMILES: [2H]C([2H])([2H])Oc1ccc2c(N3CCC4(CC3)CCN(S(N)(=O)=O)C4)ccnc2c1] |
| 421 | N-(2-methoxy-4-((7-methoxy-1,8-naphthyridin-4-yl)oxy)phenyl)sulfamide | 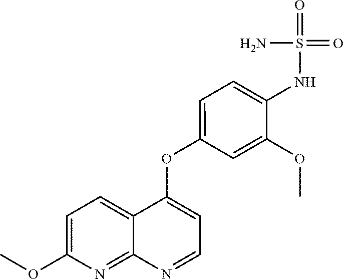 [SMILES: COc1ccc2c(Oc3ccc(NS(N)(=O)=O)c(OC)c3)ccnc2n1] |

In some embodiments, the compound disclosed herein is selected from:

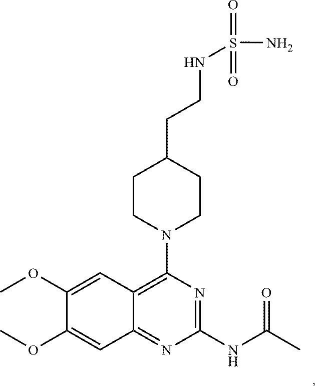

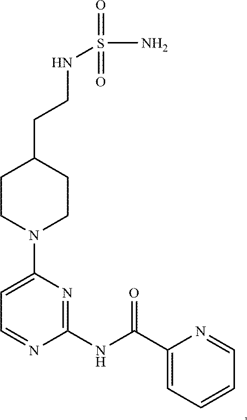

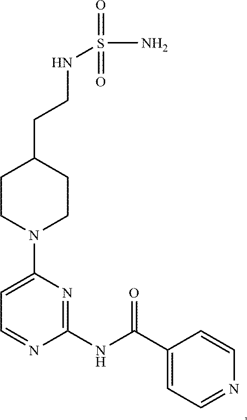

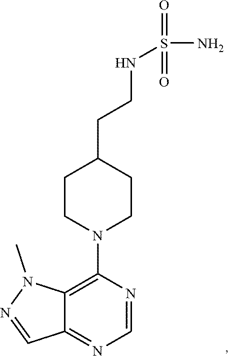

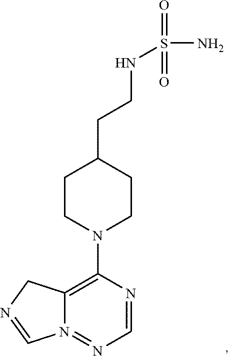

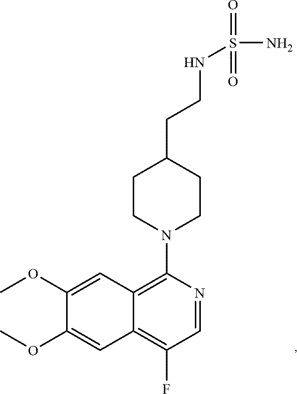

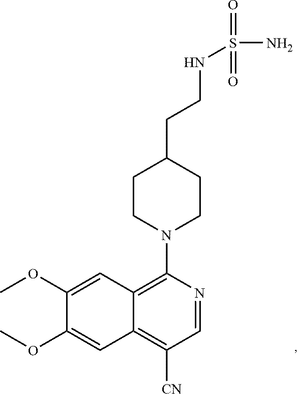

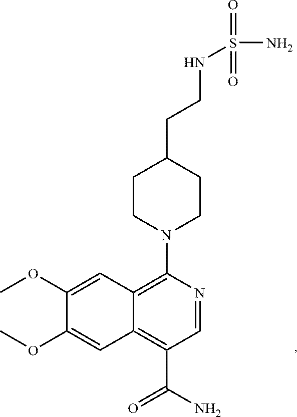

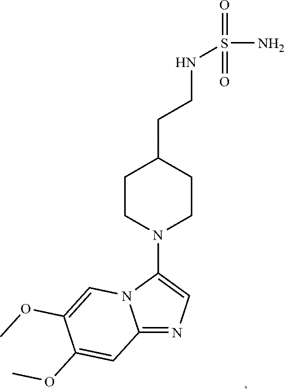

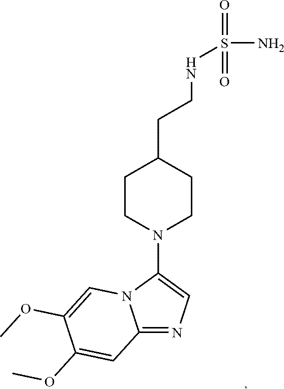

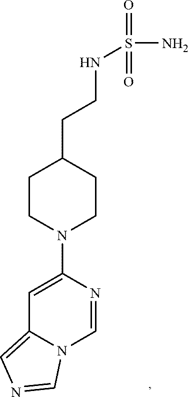

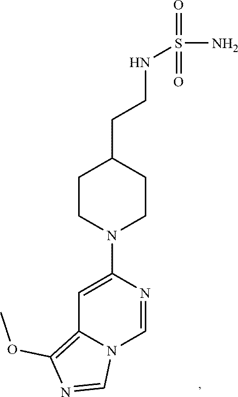

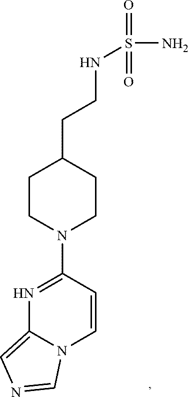

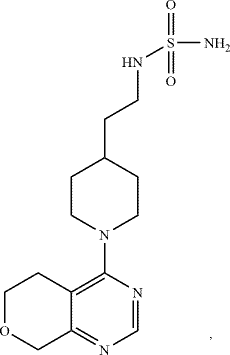

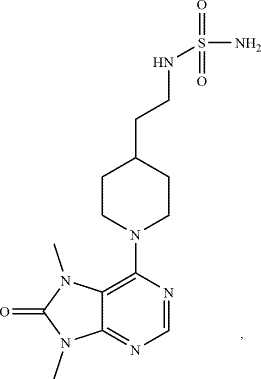

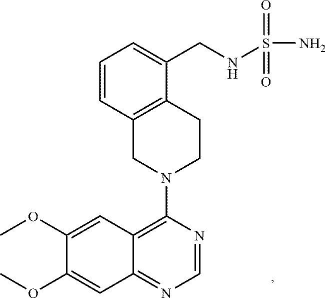

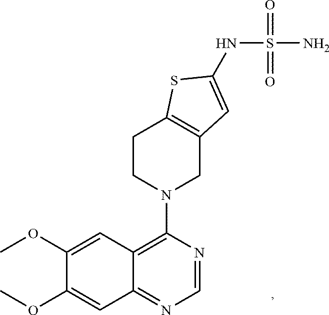

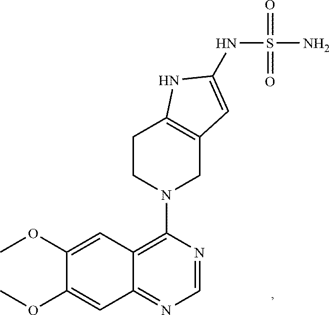

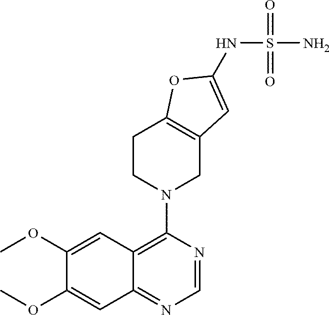 [SMILES: COc1cc2ncnc(N3CCc4oc(NS(N)(=O)=O)cc4C3)c2cc1OC]

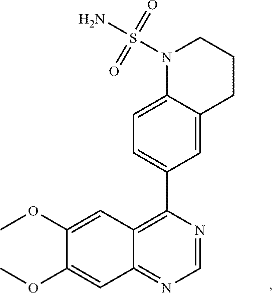

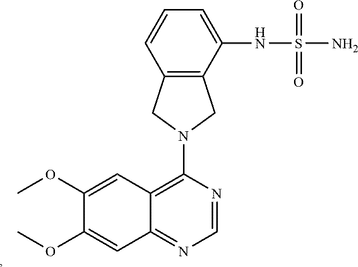

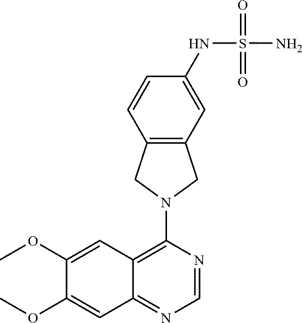 [SMILES: COc1cc2ncnc(N3Cc4ccc(NS(N)(=O)=O)cc4C3)c2cc1OC]

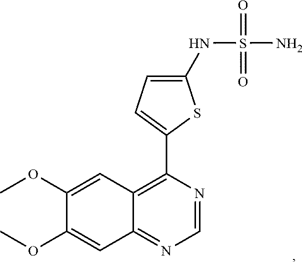

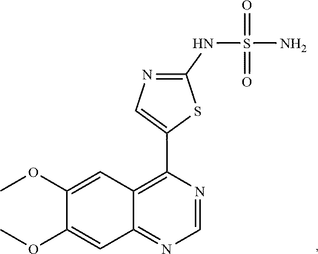

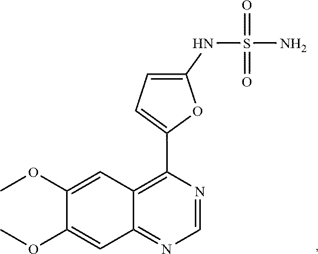

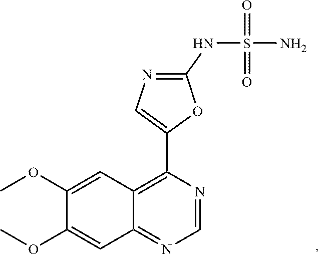

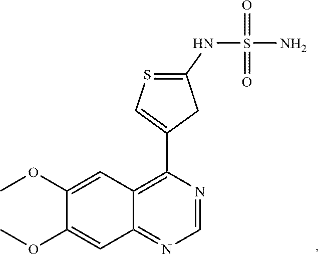

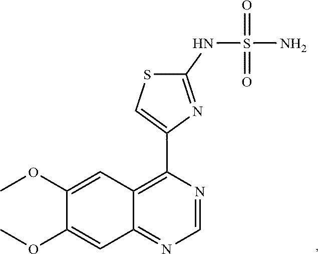

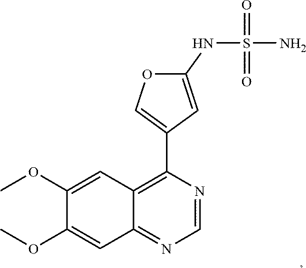

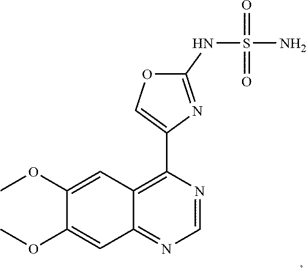

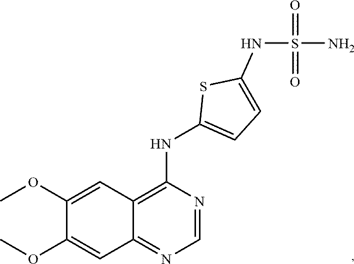

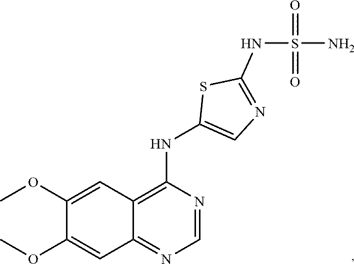

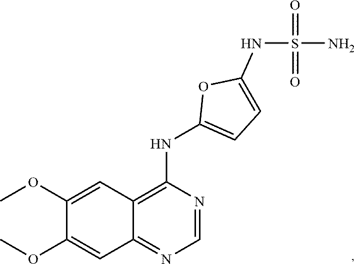

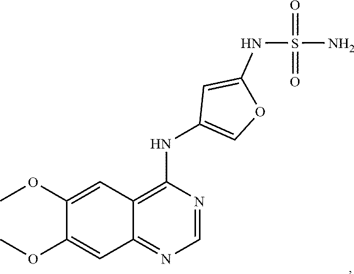

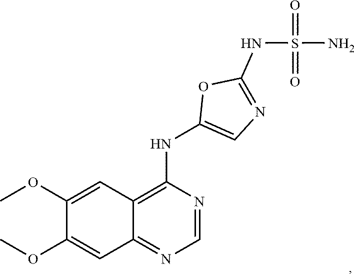

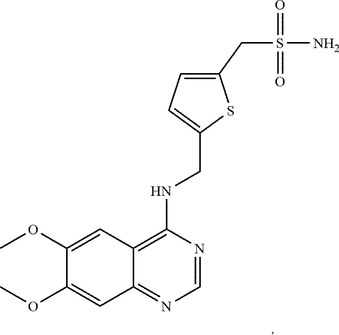

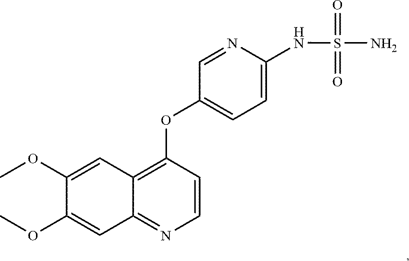

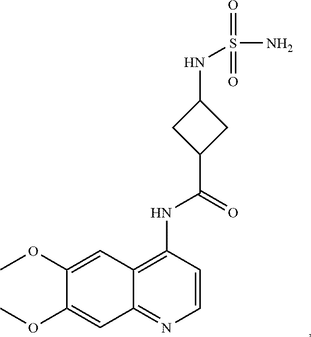

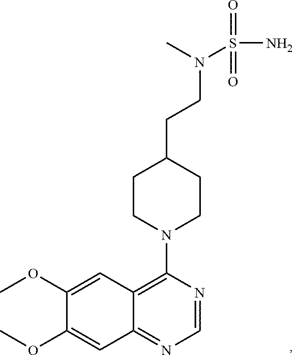

and

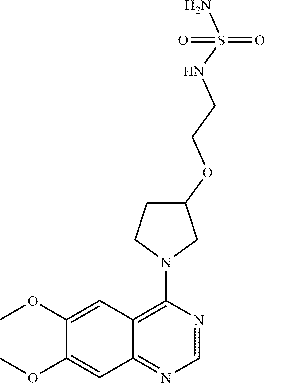

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸0, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method. In some embodiments, one or more hydrogen atoms are replaced by deuterium in any of the formula described herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai’s 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann’s Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, or steroisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient’s disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intra-arterial, intraperitoneal, or subcutaneous administration.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 µg to about 50 mg of at least one compound of the invention per kg body weight of the subject. For a 70 kg patient, dosages of from about 10 µg to about 200 mg of the compound disclosed herein would be more commonly used, depending on a subject’s physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

The compounds disclosed herein, or a pharmaceutically acceptable salt, solvate, or stereoisomer thereof, are useful as inhibitors of ENPP-1, and thereof useful in the treatment of diseases or disorders in which ENPP-1 activity plays a role. In some embodiments, disclosed herein are methods of treating a subject having cancer. In some instances, the cancer is primed with an immunogenic cell death (ICD) inducer. In other instances, the cancer is treated with an ENPP-1 inhibitor prior to administering an ICD inducer or is treated simultaneously with the ENPP-1 inhibitor and an ICD inducer.

In some embodiments, disclosed herein are methods of treating a subject having a pathogenic infection. In some instances, the method comprises administering to the subject an inhibitor of a 2’3’-cGAMP degradation polypeptide, wherein the inhibitor prevents hydrolysis of 2’3’-cGAMP and wherein the subject has an infection.

In some instances, the ENPP-1 inhibitor described herein is a competitive inhibitor. In other instances, the ENPP-1 inhibitor described herein is an allosteric inhibitor. In some cases, the ENPP-1 described herein is an irreversible inhibitor.

In some cases, the ENPP-1 inhibitor binds to one or more domains of ENPP-1. As described above, ENPP-1 comprises a catalytic domain and a nuclease-like domain. In some instances, the ENPP-1 inhibitor binds to the catalytic domain of ENPP-1. In some cases, the ENPP-1 inhibitor binds to the nuclease-like domain of ENPP-1.

In some cases, the ENPP-1 inhibitor selectively binds to a region on PDE (e.g., ENPP-1) also recognized by GMP. In some cases, a PDE inhibitor selectively binds to a region on PDE (e.g., ENPP-1) also recognized by GMP but interacts weakly with the region that is bound by AMP.

In some embodiments, a cancer described herein is a solid tumor. A solid tumor comprises neoplasms and lesions derived from cells other than blood, bone marrow, or lymphatic cells. In some cases, exemplary solid tumors include breast cancer and lung cancer.

In some embodiments, a cancer described herein is a hematologic malignancy. A hematologic malignancy comprises an abnormal cell growth of blood, bone marrow, and/or lymphatic cells. For instances, an exemplary hematologic malignancy comprises multiple myeloma. In some instances, a hematologic malignancy is a leukemia, a lymphoma or a myeloma. In some cases, a hematologic malignancy is a B-cell malignancy.

In some embodiments, a cancer described herein is a relapsed or refractory cancer. In some embodiments, a cancer described herein is a metastatic cancer.

In some embodiments, an ICD inducer comprises radiation. In some cases, the radiation comprises UV radiation. In other cases, the radiation comprises γ radiation.

In some embodiments, an ICD inducer comprises a small molecule compound or a biologic. As described above, an ICD small molecule inducer optionally comprises a chemotherapeutic agent. In some cases, the chemotherapeutic agent comprises an anthracycline. In some cases, the anthracycline is doxorubicin or mitoxantrone. In some instances, the chemotherapeutic agent comprises a cyclophosphamide. In some instances, the cyclophosphamide is mafosfamide. In some embodiments, the chemotherapeutic agent is selected from bortezomib, daunorubicin, docetaxel, oxaliplatin, paclitaxel, or a combination thereof. In some cases, the ICD inducer comprises digitoxin or digoxin. In some cases, the ICD inducer comprises septacidin. In some cases, the ICD inducer comprises a combination of cisplatin and thapsigargin. In some cases, the ICD inducer comprises a combination of cisplatin and tunicamycin.

In some embodiments, an ICD inducer comprises a biologic (e.g., a protein-payload conjugate such as trastuzumab emtansine). In some cases, the ICD inducer comprises an activator of calreticulin (CRT) exposure.

### Method of Enhancing And/or Augmenting Type IIFN Production

Also described herein are method of enhancing and/or augmenting type I interferon (IFN) production. In some instances, the method comprises an in vivo method. In some cases, the method differs from a systemic method because the production of IFNs is localized in the tumor microenvironment. In some cases, the method of enhancing type I interferon (IFN) production in a subject in need thereof, comprises administering to the subject a pharmaceutical composition comprising (i) an inhibitor of a 2’3’-cGAMP degradation polypeptide to block the hydrolysis of 2’3’-cGAMP; and (ii) a pharmaceutically acceptable excipient; wherein the presence of 2’3’-cGAMP activates the STING pathway, thereby enhancing the production of type I interferons.

In some cases, the 2’3’-cGAMP degradation polypeptide is a phosphodiesterase (PDE). In some cases, the 2’3’-cGAMP degradation polypeptide is an ectonucleotide pyrophosphatase/phosphodiesterase (ENPP) protein. In some cases, the 2’3’-cGAMP degradation polypeptide is ectonucleotide pyrophosphatase/phosphodiesterase family member 1 (ENPP-1).

In some instances, the cell has an elevated expression of PDE.

In some instances, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some embodiments, the inhibitor of a 2’3’-cGAMP degradation polypeptide is a PDE inhibitor. In some instances, the PDE inhibitor is a small molecule. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some embodiments, the PDE inhibitor binds to the catalytic domain of ENPP-1. In other embodiments, the PDE inhibitor binds to the nuclease-like domain of ENPP-1.

In some embodiments, the subject has been administered an immunogenic cell death (ICD) inducer prior to administering the inhibitor of a 2’3’-cGAMP degradation polypeptide. In other instances, the subject is administered an immunogenic cell death (ICD) inducer after administering the inhibitor of a 2’3’-cGAMP degradation polypeptide or simultaneously with the inhibitor of a 2’3’-cGAMP degradation polypeptide. In some embodiments, an ICD inducer comprises radiation. In some cases, the radiation comprises UV radiation. In other cases, the radiation comprises γ radiation.

In some embodiments, an ICD inducer comprises a small molecule compound or a biologic. As described above, an ICD small molecule inducer optionally comprises a chemotherapeutic agent. In some cases, the chemotherapeutic agent comprises an anthracycline. In some cases, the anthracycline is doxorubicin or mitoxantrone. In some instances, the chemotherapeutic agent comprises a cyclophosphamide. In some instances, the cyclophosphamide is mafosfamide. In some embodiments, the chemotherapeutic agent is selected from bortezomib, daunorubicin, docetaxel, oxaliplatin, paclitaxel, or a combination thereof. In some cases, the ICD inducer comprises digitoxin or digoxin. In some cases, the ICD inducer comprises septacidin. In some cases, the ICD inducer comprises a combination of cisplatin and thapsigargin. In some cases, the ICD inducer comprises a combination of cisplatin and tunicamycin.

In some embodiments, an ICD inducer comprises a biologic (e.g., a protein-payload conjugate such as trastuzumab emtansine). In some cases, the ICD inducer comprises an activator of calreticulin (CRT) exposure.

In some cases, the therapeutically effective amount of the inhibitor of a 2’3’-cGAMP degradation polypeptide (e.g., a ENPP-1 inhibitor) selectively inhibits hydrolysis of 2’3’-cGAMP.

In some embodiments, the therapeutically effective amount of the inhibitor of a 2’3’-cGAMP degradation polypeptide (e.g., a ENPP-1 inhibitor) further reduces ATP hydrolysis in the 2’3’-cGAMP degradation polypeptide by less than 50%, less than 40%, less than 30%, less than 20%, less than 10%, less than 5%, or by less than 1% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 50% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 40% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 30% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 20% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 10% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 5% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 4% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 3% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 2% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) reduces ATP hydrolysis in 2’3’-cGAMP degradation polypeptide by less than 1% relative to the ATP hydrolysis of a 2’3’-cGAMP degradation polypeptide in the absence of the 2’3’-cGAMP degradation polypeptide inhibitor. In some cases, the therapeutically effective amount of the inhibitor of a 2’3’-cGAMP degradation polypeptide (e.g., a ENPP-1 inhibitor) does not induce ATP hydrolysis in 2’3’-cGAMP degradation polypeptide.

In some embodiments, a cancer described herein is a solid tumor. In some cases, exemplary solid tumors include breast cancer, lung cancer and glioblastoma (e.g., glioblastoma multiforme).

In some embodiments, a cancer described herein is a hematologic malignancy. In some instances, a hematologic malignancy is a leukemia, a lymphoma or a myeloma. In some cases, a hematologic malignancy is a B-cell malignancy.

In some embodiments, a cancer described herein is a relapsed or refractory cancer.

In some embodiments, a cancer described herein is a metastatic cancer.

### Method of Inhibiting 2’3’-cGAMP Depletion

In some embodiments, further disclosed herein include methods of inhibiting depletion of 2’3’-cGAMP in a cell and selective inhibition of a 2’3’-cGAMP degradation polypeptide (e.g., a ENPP-1). In some instances, a method of inhibiting depletion of 2’3’-cGAMP in a cell comprises contacting a cell comprising a 2’3’-cGAMP degradation polypeptide with an inhibitor to generate a 2’3’-cGAMP degradation polypeptide-inhibitor adduct, thereby inhibiting the 2’3’-cGAMP degradation polypeptide from degrading 2’3’-cGAMP to prevent the depletion of 2’3’-cGAMP in the cell.

In some cases, the 2’3’-cGAMP degradation polypeptide is a phosphodiesterase (PDE). In some cases, the 2’3’-cGAMP degradation polypeptide is an ectonucleotide pyrophosphatase/phosphodiesterase (ENPP) protein. In some cases, the 2’3’-cGAMP degradation polypeptide is ectonucleotide pyrophosphatase/phosphodiesterase family member 1 (ENPP-1).

In other instances, a method of selectively inhibiting a phosphodiesterase (PDE) comprises contacting a cell characterized with an elevated population of cytosolic DNA with a catalytic domain-specific PDE inhibitor to inhibit hydrolysis of 2’3-cGAMP, wherein the PDE inhibitor has a reduced inhibition function of ATP hydrolysis of the PDE.

In additional instances, a method of selectively inhibiting a phosphodiesterase (PDE) comprises contacting a cell characterized with an elevated population of cytosolic DNA with a nuclease-like domain-specific PDE inhibitor to inhibit hydrolysis of 2’3-cGAMP, wherein the PDE inhibitor has a reduced inhibition function of ATP hydrolysis of the PDE.

In some cases, the reduced inhibition function of ATP hydrolysis is relative to the ATP hydrolysis of a PDE in the absence of the PDE inhibitor. In some cases, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some instances, the PDE inhibitor reduces ATP hydrolysis in the PDE by less than 50% relative to the ATP hydrolysis of a PDE in the absence of the PDE inhibitor. In some instances, the PDE inhibitor reduces ATP hydrolysis in the PDE by less than 40% relative to the ATP hydrolysis of a PDE in the absence of the PDE inhibitor. In some instances, the PDE inhibitor reduces ATP hydrolysis in the PDE by less than 30% relative to the ATP hydrolysis of a PDE in the absence of the PDE inhibitor. In some instances, the PDE inhibitor reduces ATP hydrolysis in the PDE by less than 20% relative to the ATP hydrolysis of a PDE in the absence of the PDE inhibitor. In some instances, the PDE inhibitor reduces ATP hydrolysis in the PDE by less than 10% relative to the ATP hydrolysis of a PDE in the absence of the PDE inhibitor. In some instances, the PDE inhibitor reduces ATP hydrolysis in the PDE by less than 5% relative to the ATP hydrolysis of a PDE in the absence of the PDE inhibitor. In some instances, the PDE inhibitor reduces ATP hydrolysis in the PDE by less than 4% relative to the ATP hydrolysis of a PDE in the absence of the PDE inhibitor. In some instances, the PDE inhibitor reduces ATP hydrolysis in the PDE by less than 3% relative to the ATP hydrolysis of a PDE in the absence of the PDE inhibitor. In some instances, the PDE inhibitor reduces ATP hydrolysis in the PDE by less than 2% relative to the ATP hydrolysis of a PDE in the absence of the PDE inhibitor. In some instances, the PDE inhibitor reduces ATP hydrolysis in the PDE by less than 1% relative to the ATP hydrolysis of a PDE in the absence of the PDE inhibitor. In some cases, the PDE inhibitor does not inhibit ATP hydrolysis of the PDE.

In some embodiments, the cell has an elevated expression of PDE.

In some embodiments, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some instances, the cell comprises a cancer cell. In some instances, the cancer cell is a solid tumor cell (e.g., a breast cancer cell, a lung cancer cell, or a cancer cell from glioblastoma). In other instances, the cancer cell is a cell from a hematologic malignancy (e.g., from a lymphoma, a leukemia, a myeloma or a B-cell malignancy).

In some embodiments, the cell comprises an effector cell. In some instances, the effector cell comprises a dendritic cell or a macrophage.

In some embodiments, the cell comprises a non-cancerous cell residing within a tumor microenvironment in which the cell comprises an elevated population of cytosolic DNA. In some cases, the cell comprises a non-cancerous cell residing within a tumor microenvironment in which the cGAS/STING pathway is activated.

In some embodiments, a subject is administered a recombinant vaccine comprising a vector that encodes a tumor antigen. In some instances, the subject is administered a recombinant vaccine prior to administering the inhibitor of a 2’3’-cGAMP degradation polypeptide. In other instances, the subject is administered a recombinant vaccine after administering the inhibitor of a 2’3’-cGAMP degradation polypeptide or simultaneously with the inhibitor of a 2’3’-cGAMP degradation polypeptide.

In some embodiments, a nucleic acid vector described herein comprise a circular plasmid or a linear nucleic acid. In some cases, the circular plasmid or linear nucleic acid is capable of directing expression of a particular nucleotide sequence in an appropriate subject cell. In some cases, the vector has a promoter operably linked to the tumor antigen-encoding nucleotide sequence, which is operably linked to termination signals. In some instances, the vector also contains sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter, which can initiate transcription only when the host cell is exposed to some particular external stimulus.

In some instances, the vector is a plasmid. In some cases, the plasmid is useful for transfecting cells with nucleic acid encoding the tumor antigen, after which the transformed host cells can be cultured and maintained under conditions wherein production of the tumor antigen takes place.

In some instances, the plasmid comprises a mammalian origin of replication in order to maintain the plasmid extrachromosomally and produce multiple copies of the plasmid in a cell. The plasmid can be pVAXI, pCEP4, or pREP4 from Invitrogen (San Diego, CA).

In some instances, the plasmid further comprises a regulatory sequence, which enables gene expression in a cell into which the plasmid is administered. In some cases, the coding sequence further comprises a codon that allows for more efficient transcription of the coding sequence in the host cell.

In some instances, the vector is a circular plasmid, which transforms a target cell by integration into the cellular genome or exist extrachromosomally (e.g., autonomous replicating plasmid with an origin of replication). Exemplary vectors include pVAX, pcDNA3.0, or provax, or any other expression vector capable of expressing DNA encoding the antigen and enabling a cell to translate the sequence to an antigen that is recognized by the immune system.

In some instances, the recombinant nucleic acid vaccine comprises a viral vector. Exemplary viral based vectors include adenoviral based, lentivirus based, adeno-associated (AAV) based, retroviral based, or poxvirus based vectors.

In some instances, the recombinant nucleic acid vaccine is a linear DNA vaccine, or linear expression cassette (“LEC”), that is capable of being efficiently delivered to a subject via electroporation and expressing one or more polypeptides disclosed herein. The LEC can be any linear DNA devoid of any phosphate backbone. The DNA can encode one or more microbial antigens. The LEC can contain a promoter, an intron, a stop codon, and/or a polyadenylation signal. In some cases, the LEC does not contain any antibiotic resistance genes and/or a phosphate backbone. In some cases, the LEC does not contain other nucleic acid sequences unrelated to the tumor antigen.

### Methods of Inhibiting 2’3’-cGAMP Depletion

In some embodiments, further disclosed herein include methods of inhibiting depletion of 2’3’-cGAMP in a cell and selective inhibition of a 2’3’-cGAMP degradation polypeptide (e.g., ENPP-1). In some embodiments, disclosed herein includes a method of inhibiting depletion of 2’3’-cGAMP in a cell infected by a pathogen, which comprises contacting the cell infected by a pathogen and expressing a 2’3’-cGAMP degradation polypeptide with an inhibitor to generate a 2’3’-cGAMP degradation polypeptide-inhibitor adduct, thereby inhibiting the 2’3’-cGAMP degradation polypeptide from degrading 2’3’-cGAMP to prevent the depletion of 2’3’-cGAMP in the cell.

In some instances, disclosed herein includes a method of selectively inhibiting a phosphodiesterase (PDE), which comprises contacting a cell characterized with an elevated population of cytosolic DNA with a PDE inhibitor to inhibit hydrolysis of 2’3-cGAMP, wherein the PDE inhibitor has a reduced function of ATP hydrolysis of the PDE, and wherein the elevated population of cytosolic DNA is generated by a virus.

In some instances, disclosed herein includes a method of selectively inhibiting a phosphodiesterase (PDE), which comprises contacting a cell characterized with an elevated population of cytosolic DNA with a catalytic domain-specific PDE inhibitor to inhibit hydrolysis of 2’3-cGAMP, wherein the PDE inhibitor has a reduced inhibition function of ATP hydrolysis of the PDE, and wherein the elevated population of cytosolic DNA is generated by a virus.

In some instances, disclosed herein includes a method of selectively inhibiting a phosphodiesterase (PDE), which comprises contacting a cell characterized with an elevated population of cytosolic DNA with a nuclease-like domain-specific PDE inhibitor to inhibit hydrolysis of 2’3-cGAMP, wherein the PDE inhibitor has a reduced inhibition function of ATP hydrolysis of the PDE, and wherein the elevated population of cytosolic DNA is generated by a virus.

In some embodiments, disclosed herein includes a method of selectively inhibiting a phosphodiesterase (PDE), which comprises contacting a cell characterized with an elevated population of cytosolic DNA with a PDE inhibitor to inhibit hydrolysis of 2’3-cGAMP, wherein the PDE inhibitor has a reduced inhibition function ATP hydrolysis of the PDE, and wherein the elevated population of cytosolic DNA is generated by a recombinant DNA vaccine.

In some embodiments, disclosed herein includes a method of selectively inhibiting a phosphodiesterase (PDE), which comprises contacting a cell characterized with an elevated population of cytosolic DNA with a catalytic domain-specific PDE inhibitor to inhibit hydrolysis of 2’3-cGAMP, wherein the PDE inhibitor has a reduced inhibition function of ATP hydrolysis of the PDE, and wherein the elevated population of cytosolic DNA is generated by a recombinant DNA vaccine.

In some embodiments, disclosed herein includes a method of selectively inhibiting a phosphodiesterase (PDE), which comprises contacting a cell characterized with an elevated population of cytosolic DNA with a nuclease-like domain-specific PDE inhibitor to inhibit hydrolysis of 2’3-cGAMP, wherein the PDE inhibitor has a reduced inhibition function of ATP hydrolysis of the PDE, and wherein the elevated population of cytosolic DNA is generated by a recombinant DNA vaccine.

In some cases, the 2’3’-cGAMP degradation polypeptide is a phosphodiesterase (PDE). In some cases, the 2’3’-cGAMP degradation polypeptide is an ectonucleotide pyrophosphatase/phosphodiesterase (ENPP) protein. In some cases, the 2’3’-cGAMP degradation polypeptide is ectonucleotide pyrophosphatase/phosphodiesterase family member 1 (ENPP-1).

In some instances, a method of selectively inhibiting a phosphodiesterase (PDE) comprises contacting a cell characterized with an elevated population of cytosolic DNA with a PDE inhibitor to inhibit hydrolysis of 2’3-cGAMP, wherein the PDE inhibitor has a reduced inhibition function of ATP hydrolysis of the PDE. In some cases, the PDE inhibitor binds to the catalytic domain of ENPP-1. In some cases, the PDE inhibitor binds to the nuclease-like domain of ENPP-1.

In some embodiments, the infection is a viral infection, e.g., an infection from a DNA virus or a retrovirus. In some cases, the viral infection is due to herpes simplex virus 1 (HSV-1), murine gamma-herpesvirus 68 (MHV68), Kaposi’s sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some instances, the infection is a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae.

In some instances, the cytosolic DNA comprises viral DNA. In some cases, the elevated population of cytosolic DNA is due to a viral infection to the host cell. In other cases, the elevated population of the cytosolic DNA is due to delivery of viral DNA through a virus-like particle (VLP).

In some instances, the elevated population of cytosolic DNA is due to a recombinant DNA vaccine, which comprises a DNA vector encoding a viral antigen. In some cases, the viral antigen is derived from a DNA virus. In other cases, the viral antigen is derived from a retrovirus. In some cases, the viral antigen is derived from herpes simplex virus 1 (HSV-1), murine gamma-herpesvirus 68 (MHV68), Kaposi’s sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some cases, the recombinant DNA vaccine comprise a DNA vector that encodes a bacterial antigen, e.g., derived from a Gram-negative bacterium or a Gram-positive bacterium. In some cases, the bacterial antigen is derived from Listeria monocytogenes, Mycobacterium tuberculosis, Francisella novicida, Legionella pneumophila, Chlamydia trachomatis, Streptococcus pneumoniae, or Neisseria gonorrhoeae.

In some embodiments, a DNA vector described herein comprise a circular plasmid or a linear nucleic acid. In some cases, the circular plasmid or linear nucleic acid is capable of directing expression of a particular nucleotide sequence in an appropriate subject cell. In some cases, the vector has a promoter operably linked to the microbial antigen-encoding nucleotide sequence, which is operably linked to termination signals. In some instances, the vector also contains sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter, which can initiate transcription only when the host cell is exposed to some particular external stimulus.

In some instances, the vector is a plasmid. In some cases, the plasmid is useful for transfecting cells with nucleic acid encoding the microbial antigen, which the transformed host cells can be cultured and maintained under conditions wherein production of the microbial antigen takes place.

In some instances, the plasmid comprises a mammalian origin of replication in order to maintain the plasmid extrachromosomally and produce multiple copies of the plasmid in a cell. The plasmid can be pVAXI, pCEP4, or pREP4 from Invitrogen (San Diego, CA).

In some instances, the plasmid further comprises a regulatory sequence, which enables gene expression in a cell into which the plasmid is administered. In some cases, the coding sequence further comprises a codon that allows for more efficient transcription of the coding sequence in the host cell.

In some instances, the vector is a circular plasmid, which transforms a target cell by integration into the cellular genome or exist extrachromosomally (e.g., autonomous replicating plasmid with an origin of replication). Exemplary vectors include pVAX, pcDNA3.0, or provax, or any other expression vector capable of expressing DNA encoding the antigen and enabling a cell to translate the sequence to an antigen that is recognized by the immune system.

In some instances, the recombinant nucleic acid vaccine comprises a viral vector. Exemplary viral based vectors include adenoviral based, lentivirus based, adeno-associated (AAV) based, retroviral based, or poxvirus based vectors.

In some instances, the recombinant DNA vaccine is a linear DNA vaccine, or linear expression cassette (“LEC”), that is capable of being efficiently delivered to a subject via electroporation and expressing one or more polypeptides disclosed herein. The LEC can be any linear DNA devoid of any phosphate backbone. The DNA can encode one or more microbial antigens. The LEC can contain a promoter, an intron, a stop codon, and/or a polyadenylation signal. In some cases, the LEC does not contain any antibiotic resistance genes and/or a phosphate backbone. In some cases, the LEC does not contain other nucleic acid sequences unrelated to the microbial antigen.

### Method of Activating a STING Protein Dimer

In some embodiments, a method of stabilizing a stimulator of interferon genes (STING) protein dimer in a cell comprises (a) contacting a cell characterized with an elevated expression of a phosphodiesterase (PDE) or an elevated population of cytosolic DNA with a PDE inhibitor to inhibit hydrolysis of 2’3’-cGAMP; and (b) interacting 2’3’-cGAMP to a STING protein dimer to generate a 2’3’-cGAMP-STING complex, thereby stabilizing the STING protein dimer. In some instances, interacting of 2’3’-cGAMP to a STING protein dimer to generate a 2’3’-cGAMP-STING complex further activates the STING protein dimer. In some cases, activation of the STING protein dimer further leads to upregulating the production of type I interferon (IFN). In some cases, the production of IFNs is localized in a tumor microenvironment.

In some instances, the cell has an elevated population of cytosolic DNA. In some cases, the elevated population of cytosolic DNA is generated by an ICD-mediated event. In other cases, the elevated population of cytosolic DNA is generated by DNA structure-specific endonuclease MUS81.

In some cases, the 2’3’-cGAMP degradation polypeptide is a phosphodiesterase (PDE). In some cases, the 2’3’-cGAMP degradation polypeptide is an ectonucleotide pyrophosphatase/phosphodiesterase (ENPP) protein. In some cases, the 2’3’-cGAMP degradation polypeptide is ectonucleotide pyrophosphatase/phosphodiesterase family member 1 (ENPP-1).

In some instances, the cell comprises a cancer cell. In some instances, the cancer cell is a solid tumor cell (e.g., a breast cancer cell, a lung cancer cell, or a cancer cell from glioblastoma). In other instances, the cancer cell is a cell from a hematologic malignancy (e.g., from a lymphoma, a leukemia, a myeloma or a B-cell malignancy).

In some embodiments, the cell comprises an effector cell. In some instances, the effector cell comprises a dendritic cell or a macrophage.

In some embodiments, the cell comprises a non-cancerous cell residing within a tumor microenvironment in which the cell comprises an elevated population of cytosolic DNA. In some cases, the cell comprises a non-cancerous cell residing within a tumor microenvironment in which the cGAS/STING pathway is activated.

### Dosing Regimen

In some embodiments, the ENPP-1 inhibitor described herein is administered for therapeutic applications. In some embodiments, the ENPP-1 inhibitor is administered once per day, twice per day, three times per day or more. The ENPP-1 inhibitor is administered daily, every day, every alternate day, five days a week, once a week, every other week, two weeks per month, three weeks per month, once a month, twice a month, three times per month, or more. The ENPP-1 inhibitor is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient’s status does improve, upon the doctor’s discretion the administration of the ENPP-1 inhibitor is given continuously; alternatively, the dose of the ENPP-1 inhibitor being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient’s conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of the ENPP-1 inhibitor varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some embodiments, the ENPP-1 inhibitor is administered to a subject at least 0.5, 1, 1.5, 2, 3, 4, 5, 6, 7, 8, 9, 10, 11, 12, 18, 24, 36, or 48 hours after administration of the ICD inducer. In some cases, the ENPP-1 inhibitor is administered to the subject at least 0.5 hour after administration of the ICD inducer. In some cases, the ENPP-1 inhibitor is administered to the subject at least 1 hour after administration of the ICD inducer. In some cases, the ENPP-1 inhibitor is administered to the subject at least 1.5 hours after administration of the ICD inducer. In some cases, the ENPP-1 inhibitor is administered to the subject at least 2 hours after administration of the ICD inducer. In some cases, the ENPP-1 inhibitor is administered to the subject at least 3 hours after administration of the ICD inducer. In some cases, the ENPP-1 inhibitor is administered to the subject at least 4 hours after administration of the ICD inducer. In some cases, the ENPP-1 inhibitor is administered to the subject at least 5 hours after administration of the ICD inducer. In some cases, the ENPP-1 inhibitor is administered to the subject at least 6 hours after administration of the ICD inducer. In some cases, the ENPP-1 inhibitor is administered to the subject at least 7 hours after administration of the ICD inducer. In some cases, the ENPP-1 inhibitor is administered to the subject at least 8 hours after administration of the ICD inducer. In some cases, the ENPP-1 inhibitor is administered to the subject at least 9 hours after administration of the ICD inducer. In some cases, the ENPP-1 inhibitor is administered to the subject at least 10 hours after administration of the ICD inducer. In some cases, the ENPP-1 inhibitor is administered to the subject at least 11 hours after administration of the ICD inducer. In some cases, the ENPP-1 inhibitor is administered to the subject at least 12 hours after administration of the ICD inducer. In some cases, the ENPP-1 inhibitor is administered to the subject at least 18 hours after administration of the ICD inducer. In some cases, the ENPP-1 inhibitor is administered to the subject at least 24 hours after administration of the ICD inducer. In some cases, the ENPP-1 inhibitor is administered to the subject at least 36 hours after administration of the ICD inducer. In some cases, the ENPP-1 inhibitor is administered to the subject at least 48 hours after administration of the ICD inducer.

In some embodiments, the ENPP-1 inhibitor is administered to a subject at least 1, 2, 3, 4, 5, 6, 7, 8, 9, 10, 11, 12, 13, 14, 28, 30, or 40 days after administration of the ICD inducer. In some cases, the ENPP-1 inhibitor is administered to the subject at least 1 day after administration of the ICD inducer. In some cases, the ENPP-1 inhibitor is administered to the subject at least 2 days after administration of the ICD inducer. In some cases, the ENPP-1 inhibitor is administered to the subject at least 3 days after administration of the ICD inducer. In some cases, the ENPP-1 inhibitor is administered to the subject at least 4 days after administration of the ICD inducer. In some cases, the ENPP-1 inhibitor is administered to the subject at least 5 days after administration of the ICD inducer. In some cases, the ENPP-1 inhibitor is administered to the subject at least 6 days after administration of the ICD inducer. In some cases, the ENPP-1 inhibitor is administered to the subject at least 7 days after administration of the ICD inducer. In some cases, the ENPP-1 inhibitor is administered to the subject at least 8 days after administration of the ICD inducer. In some cases, the ENPP-1 inhibitor is administered to the subject at least 9 days after administration of the ICD inducer. In some cases, the ENPP-1 inhibitor is administered to the subject at least 10 days after administration of the ICD inducer. In some cases, the ENPP-1 inhibitor is administered to the subject at least 11 days after administration of the ICD inducer. In some cases, the ENPP-1 inhibitor is administered to the subject at least 12 days after administration of the ICD inducer. In some cases, the ENPP-1 inhibitor is administered to the subject at least 13 days after administration of the ICD inducer. In some cases, the ENPP-1 inhibitor is administered to the subject at least 14 days after administration of the ICD inducer. In some cases, the ENPP-1 inhibitor is administered to the subject at least 28 days after administration of the ICD inducer. In some cases, the ENPP-1 inhibitor is administered to the subject at least 30 days after administration of the ICD inducer.

In some embodiments, the ENPP-1 inhibitor is administered to a subject at least 0.5, 1, 1.5, 2, 3, 4, 5, 6, 7, 8, 9, 10, 11, 12, 18, 24, 36, or 48 hours prior to administration of the ICD inducer. In some cases, the ENPP-1 inhibitor is administered to the subject at least 0.5 hour prior to administration of the ICD inducer. In some cases, the ENPP-1 inhibitor is administered to the subject at least 1 hour prior to administration of the ICD inducer. In some cases, the ENPP-1 inhibitor is administered to the subject at least 1.5 hours prior to administration of the ICD inducer. In some cases, the ENPP-1 inhibitor is administered to the subject at least 2 hours prior to administration of the ICD inducer. In some cases, the ENPP-1 inhibitor is administered to the subject at least 3 hours prior to administration of the ICD inducer. In some cases, the ENPP-1 inhibitor is administered to the subject at least 4 hours prior to administration of the ICD inducer. In some cases, the ENPP-1 inhibitor is administered to the subject at least 5 hours prior to administration of the ICD inducer. In some cases, the ENPP-1 inhibitor is administered to the subject at least 6 hours prior to administration of the ICD inducer. In some cases, the ENPP-1 inhibitor is administered to the subject at least 7 hours prior to administration of the ICD inducer. In some cases, the ENPP-1 inhibitor is administered to the subject at least 8 hours prior to administration of the ICD inducer. In some cases, the ENPP-1 inhibitor is administered to the subject at least 9 hours prior to administration of the ICD inducer. In some cases, the ENPP-1 inhibitor is administered to the subject at least 10 hours prior to administration of the ICD inducer. In some cases, the ENPP-1 inhibitor is administered to the subject at least 11 hours prior to administration of the ICD inducer. In some cases, the ENPP-1 inhibitor is administered to the subject at least 12 hours prior to administration of the ICD inducer. In some cases, the ENPP-1 inhibitor is administered to the subject at least 18 hours prior to administration of the ICD inducer. In some cases, the ENPP-1 inhibitor is administered to the subject at least 24 hours prior to administration of the ICD inducer. In some cases, the ENPP-1 inhibitor is administered to the subject at least 36 hours prior to administration of the ICD inducer. In some cases, the ENPP-1 inhibitor is administered to the subject at least 48 hours prior to administration of the ICD inducer.

In some embodiments, the ENPP-1 inhibitor is administered to a subject at least 1, 2, 3, 4, 5, 6, 7, 8, 9, 10, 11, 12, 13, 14, 28, 30, or 40 days prior to administration of the ICD inducer. In some cases, the ENPP-1 inhibitor is administered to the subject at least 1 day prior to administration of the ICD inducer. In some cases, the ENPP-1 inhibitor is administered to the subject at least 2 days prior to administration of the ICD inducer. In some cases, the ENPP-1 inhibitor is administered to the subject at least 3 days prior to administration of the ICD inducer. In some cases, the ENPP-1 inhibitor is administered to the subject at least 4 days prior to administration of the ICD inducer. In some cases, the ENPP-1 inhibitor is administered to the subject at least 5 days prior to administration of the ICD inducer. In some cases, the ENPP-1 inhibitor is administered to the subject at least 6 days prior to administration of the ICD inducer. In some cases, the ENPP-1 inhibitor is administered to the subject at least 7 days prior to administration of the ICD inducer. In some cases, the ENPP-1 inhibitor is administered to the subject at least 8 days prior to administration of the ICD inducer. In some cases, the ENPP-1 inhibitor is administered to the subject at least 9 days prior to administration of the ICD inducer. In some cases, the ENPP-1 inhibitor is administered to the subject at least 10 days prior to administration of the ICD inducer. In some cases, the ENPP-1 inhibitor is administered to the subject at least 11 days prior to administration of the ICD inducer. In some cases, the ENPP-1 inhibitor is administered to the subject at least 12 days prior to administration of the ICD inducer. In some cases, the ENPP-1 inhibitor is administered to the subject at least 13 days prior to administration of the ICD inducer. In some cases, the ENPP-1 inhibitor is administered to the subject at least 14 days prior to administration of the ICD inducer. In some cases, the ENPP-1 inhibitor is administered to the subject at least 28 days prior to administration of the ICD inducer. In some cases, the ENPP-1 inhibitor is administered to the subject at least 30 days prior to administration of the ICD inducer.

In some cases, the ENPP-1 inhibitor is administered simultaneously with an ICD inducer.

In some cases, the ENPP-1 inhibitor is administered continuously for 1, 2, 3, 4, 5, 6, 7, 8, 9, 10, 14, 15, 28, 30, or more days. In some instances, the ENPP-1 inhibitor is administered continuously for 1 or more days. In some instances, the ENPP-1 inhibitor is administered continuously for 2 or more days. In some instances, the ENPP-1 inhibitor is administered continuously for 3 or more days. In some instances, the ENPP-1 inhibitor is administered continuously for 4 or more days. In some instances, the ENPP-1 inhibitor is administered continuously for 5 or more days. In some instances, the ENPP-1 inhibitor is administered continuously for 6 or more days. In some instances, the ENPP-1 inhibitor is administered continuously for 7 or more days. In some instances, the ENPP-1 inhibitor is administered continuously for 8 or more days. In some instances, the ENPP-1 inhibitor is administered continuously for 9 or more days. In some instances, the ENPP-1 inhibitor is administered continuously for 10 or more days. In some instances, the ENPP-1 inhibitor is administered continuously for 14 or more days. In some instances, the ENPP-1 inhibitor is administered continuously for 15 or more days. In some instances, the ENPP-1 inhibitor is administered continuously for 28 or more days. In some instances, the ENPP-1 inhibitor is administered continuously for 30 or more days.

In some cases, the ENPP-1 inhibitor is administered at predetermined time intervals for 1, 2, 3, 4, 5, 6, 7, 8, 9, 10, 14, 15, 28, 30, or more days. In some instances, the ENPP-1 inhibitor is administered at predetermined time intervals for 1 or more days. In some instances, the ENPP-1 inhibitor is administered at predetermined time intervals for 2 or more days. In some instances, the ENPP-1 inhibitor is administered at predetermined time intervals for 3 or more days. In some instances, the ENPP-1 inhibitor is administered at predetermined time intervals for 4 or more days. In some instances, the ENPP-1 inhibitor is administered at predetermined time intervals for 5 or more days. In some instances, the ENPP-1 inhibitor is administered at predetermined time intervals for 6 or more days. In some instances, the ENPP-1 inhibitor is administered at predetermined time intervals for 7 or more days. In some instances, the ENPP-1 inhibitor is administered at predetermined time intervals for 8 or more days. In some instances, the ENPP-1 inhibitor is administered at predetermined time intervals for 9 or more days. In some instances, the ENPP-1 inhibitor is administered at predetermined time intervals for 10 or more days. In some instances, the ENPP-1 inhibitor is administered at predetermined time intervals for 14 or more days. In some instances, the ENPP-1 inhibitor is administered at predetermined time intervals for 15 or more days. In some instances, the ENPP-1 inhibitor is administered at predetermined time intervals for 28 or more days. In some instances, the ENPP-1 inhibitor is administered at predetermined time intervals for 30 or more days.

In some embodiments, the ENPP-1 inhibitor is administered at predetermined time intervals for 1, 2, 3, 4, 5, 6, 7, 8, 9, 10, 11, 12, 24, 36, or more months. In some instances, the ENPP-1 inhibitor is administered at predetermined time intervals for 1 or more month. In some instances, the ENPP-1 inhibitor is administered at predetermined time intervals for 2 or more months. In some instances, the ENPP-1 inhibitor is administered at predetermined time intervals for 3 or more months. In some instances, the ENPP-1 inhibitor is administered at predetermined time intervals for 4 or more months. In some instances, the ENPP-1 inhibitor is administered at predetermined time intervals for 5 or more months. In some instances, the ENPP-1 inhibitor is administered at predetermined time intervals for 6 or more months. In some instances, the ENPP-1 inhibitor is administered at predetermined time intervals for 7 or more months. In some instances, the ENPP-1 inhibitor is administered at predetermined time intervals for 8 or more months. In some instances, the ENPP-1 inhibitor is administered at predetermined time intervals for 9 or more months. In some instances, the ENPP-1 inhibitor is administered at predetermined time intervals for 10 or more months. In some instances, the ENPP-1 inhibitor is administered at predetermined time intervals for 11 or more months. In some instances, the ENPP-1 inhibitor is administered at predetermined time intervals for 12 or more months. In some instances, the ENPP-1 inhibitor is administered at predetermined time intervals for 24 or more months. In some instances, the ENPP-1 inhibitor is administered at predetermined time intervals for 36 or more months.

In some cases, the ENPP-1 inhibitor is administered intermittently for 1, 2, 3, 4, 5, 6, 7, 8, 9, 10, 14, 15, 28, 30, or more days. In some instances, the ENPP-1 inhibitor is administered intermittently for 1 or more days. In some instances, the ENPP-1 inhibitor is administered intermittently for 2 or more days. In some instances, the ENPP-1 inhibitor is administered intermittently for 3 or more days. In some instances, the ENPP-1 inhibitor is administered intermittently for 4 or more days. In some instances, the ENPP-1 inhibitor is administered intermittently for 5 or more days. In some instances, the ENPP-1 inhibitor is administered intermittently for 6 or more days. In some instances, the ENPP-1 inhibitor is administered intermittently for 7 or more days. In some instances, the ENPP-1 inhibitor is administered intermittently for 8 or more days. In some instances, the ENPP-1 inhibitor is administered intermittently for 9 or more days. In some instances, the ENPP-1 inhibitor is administered intermittently for 10 or more days. In some instances, the ENPP-1 inhibitor is administered intermittently for 14 or more days. In some instances, the ENPP-1 inhibitor is administered intermittently for 15 or more days. In some instances, the ENPP-1 inhibitor is administered intermittently for 28 or more days. In some instances, the ENPP-1 inhibitor is administered intermittently for 30 or more days.

In some embodiments, the ENPP-1 inhibitor is administered for at least 1 cycle, 2 cycles, 3 cycles, 4 cycles, 5 cycles, 6 cycles, 7 cycles, 8 cycles, or more. In some embodiments, the ENPP-1 inhibitor is administered for at least 1 cycle, 2 cycles, 3 cycles, 4 cycles, 5 cycles, or more. In some cases, the ENPP-1 inhibitor is administered for at least 1 cycle. In some cases, the ENPP-1 inhibitor is administered for at least 2 cycles. In some cases, the ENPP-1 inhibitor is administered for at least 3 cycles. In some cases, the ENPP-1 inhibitor is administered for at least 4 cycles. In some cases, the ENPP-1 inhibitor is administered for at least 5 cycles. In some cases, the ENPP-1 inhibitor is administered for at least 6 cycles. In some cases, the ENPP-1 inhibitor is administered for at least 7 cycles. In some cases, the ENPP-1 inhibitor is administered for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, the ENPP-1 inhibitor is administered simultaneously or sequentially with an ICD inducer for at least 1 cycle, 2 cycles, 3 cycles, 4 cycles, 5 cycles, 6 cycles, 7 cycles, 8 cycles, or more. In some embodiments, the ENPP-1 inhibitor is administered simultaneously or sequentially with an ICD inducer for at least 1 cycle, 2 cycles, 3 cycles, 4 cycles, 5 cycles, or more. In some cases, the ENPP-1 inhibitor is administered simultaneously or sequentially with an ICD inducer for at least 1 cycle. In some cases, the ENPP-1 inhibitor is administered simultaneously or sequentially with an ICD inducer for at least 2 cycles. In some cases, the ENPP-1 inhibitor is administered simultaneously or sequentially with an ICD inducer for at least 3 cycles. In some cases, the ENPP-1 inhibitor is administered simultaneously or sequentially with an ICD inducer for at least 4 cycles. In some cases, the ENPP-1 inhibitor is administered simultaneously or sequentially with an ICD inducer for at least 5 cycles. In some cases, the ENPP-1 inhibitor is administered simultaneously or sequentially with an ICD inducer for at least 6 cycles. In some cases, the ENPP-1 inhibitor is administered simultaneously or sequentially with an ICD inducer for at least 7 cycles. In some cases, the ENPP-1 inhibitor is administered simultaneously or sequentially with an ICD inducer for at least 8 cycles. In some instances, a cycle comprises 14 to 28 days. In some cases, a cycle comprises 14 days. In some cases, a cycle comprises 21 days. In some cases, a cycle comprises 28 days.

In some embodiments, the ENPP-1 inhibitor is administered simultaneously or sequentially with an ICD inducer for at least 1, 5, 10, 14, 15, 20, 21, 28, 30, 60, or 90 days. In some cases, the ENPP-1 inhibitor is administered simultaneously or sequentially with an ICD inducer for at least 1 day. In some cases, the ENPP-1 inhibitor is administered simultaneously or sequentially with an ICD inducer for at least 5 days. In some cases, the ENPP-1 inhibitor is administered simultaneously or sequentially with an ICD inducer for at least 10 days. In some cases, the ENPP-1 inhibitor is administered simultaneously or sequentially with an ICD inducer for at least 14 days. In some cases, the ENPP-1 inhibitor is administered simultaneously or sequentially with an ICD inducer for at least 15 days. In some cases, the ENPP-1 inhibitor is administered simultaneously or sequentially with an ICD inducer for at least 20 days. In some cases, the ENPP-1 inhibitor is administered simultaneously or sequentially with an ICD inducer for at least 21 days. In some cases, the ENPP-1 inhibitor is administered simultaneously or sequentially with an ICD inducer for at least 28 days. In some cases, the ENPP-1 inhibitor is administered simultaneously or sequentially with an ICD inducer for at least 30 days. In some cases, the ENPP-1 inhibitor is administered simultaneously or sequentially with an ICD inducer for at least 60 days. In some cases, the ENPP-1 inhibitor is administered simultaneously or sequentially with an ICD inducer for at least 90 days.

In some instances, the ENPP-1 inhibitor is administered to a subject at a therapeutically effective amount. For example, the therapeutically effective amount is optionally administered in 1 dose, 2 doses, 3 doses, 4 doses, 5 doses, 6 doses or more. In some instances, the therapeutically effective amount of the ENPP-1 inhibitor is administered to a subject in 1 dose. In some instances, the therapeutically effective amount of the ENPP-1 inhibitor is administered to a subject in 2 or more doses. In some instances, the therapeutically effective amount of the ENPP-1 inhibitor is administered to a subject in 3 or more doses. In some instances, the therapeutically effective amount of the ENPP-1 inhibitor is administered to a subject in 4 or more doses. In some instances, the therapeutically effective amount of the ENPP-1 inhibitor is administered to a subject in 5 or more doses. In some instances, the therapeutically effective amount of the ENPP-1 inhibitor is administered to a subject in 6 or more doses.

In some cases, the therapeutically effective amount of the ENPP-1 inhibitor selectively inhibits hydrolysis of 2’3’-cGAMP.

In some embodiments, the therapeutically effective amount of the ENPP-1 inhibitor further reduces ATP hydrolysis in ENPP-1 by less than 50%, less than 40%, less than 30%, less than 20%, less than 10%, less than 5%, or by less than 1% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 50% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 40% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 30% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 20% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 10% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 5% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 4% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 3% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 2% relative to the ATP hydrolysis of the ENPP-1 in the absence of the ENPP-1 inhibitor. In some cases, the therapeutically effective amount of the ENPP-1 inhibitor reduces ATP hydrolysis in ENPP-1 by less than 1% relative to the ATP hydrolysis of the ENPP-1 in the absence of the ENPP-1 inhibitor. In some cases, the therapeutically effective amount of the ENPP-1 inhibitor does not induce ATP hydrolysis in ENPP-1.

### Additional Therapeutic Agents

In some embodiments, one or more methods described herein further comprising administering an additional therapeutic agent. In some instances, the additional therapeutic agent is a chemotherapeutic agent. In some instances, the additional therapeutic agent is an immune checkpoint inhibitor. An exemplary immune checkpoint inhibitor comprises an inhibitor of PD1, an inhibitor of PD-L1, an inhibitor of TIM or an inhibitor of TIGIT. In some cases, the subject has a resistance to an immune checkpoint inhibitor prior to the administration of the inhibitor of PDE. In some cases, the ENPP-1 inhibitor and the additional therapeutic agent is administered simultaneously. In other cases, the ENPP-1 inhibitor and the additional therapeutic agent is administered sequentially. In some instances, the ENPP-1 inhibitor is administered before administering the additional therapeutic agent. In other instances, the ENPP-1 inhibitor is administered after administering the additional therapeutic agent.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include the ENPP-1 inhibitor, optionally with one or more additional therapeutic agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

Example 1: Synthesis of N-(2-(1-(2-amino-6,7-dimethoxyquinazolin-4-yl)piperidin-4-yl)ethyl)sulfamide

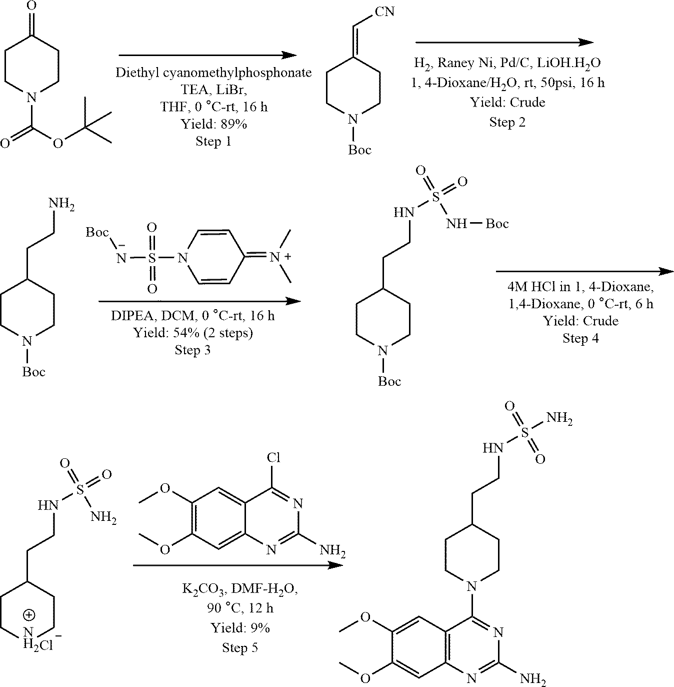

### Step 1: Synthesis of Tert-Butyl 4-(Cyanomethylene)Piperidine-1-Carboxylate

Procedure: To a stirred solution of LiBr (2.6 g, 30.15 mmol) in THF (100 mL) were added Diethyl cyanomethyl-phosphonate (4.89 mL, 30.15 mmol), triethylamine (6.76 mL, 50.25 mmol) at 0° C. Resulting mixture was stirred for 10 mins at same temperature and tert-butyl 4-oxopiperidine-1-carboxylate (5 g, 25.15 mmol) was added. Reaction mixture was stirred for 16 h at rt. The progress of the reaction was monitored by TLC. Reaction mixture was diluted with ethyl acetate (50 mL) washed with saturated sodium bicarbonate (30 mL) followed by brine (30 mL), dried over anhydrous sodium sulphate, filtered and evaporated under reduced pressure. The crude residue was purified by combiflash using 10% ethyl acetate in hexane to afford tert-butyl 4-(cyanomethylene)piperidine-1-carboxylate as white solid (5.0 g, 89%). ¹H NMR (400 MHz CDCl₃): δ 5.19 (s, 1 H), 3.53 - 3.48 (m, 4 H), 2.56 (t, J= 6.0 Hz, 2 H), 2.32 (t, J = 5.6 Hz, 2H), 1.47 (s, 9 H).

### Step 2: Synthesis of Tert-Butyl 4-(2-Aminoethyl)Piperidine-1-Carboxylate

Procedure: To a stirred solution of tert-butyl 4-(cyanomethylene) piperidine-1-carboxylate (5.0 g, 22.52 mmol) in 1,4-dioxane (100 mL)/H₂O (30 mL), were added lithium hydroxide monohydrate (2.08 g, 49.54 mmol), Raney Ni (5 g), 10%Pd/C (1.5 g). Reaction mixture was stirred at room temperature for 16 h at 50 psi under H₂ atmosphere. The progress of the reaction was monitored by TLC. The reaction mixture was filtered through a celite bed and the filtrate obtained was evaporated under reduced pressure to afford tert-butyl 4-(2-aminoethyl)piperidine-1-carboxylate as a pale brown liquid (5.0 g, crude). LC-MS (ES) m/z = 228.9 [M+H]⁺

### Step 3: Synthesis of Tert-Butyl 4-(2-((N-(Tert-Butoxycarbonyl)Sulfamoyl)Amino)Ethyl) Piperidine-1-Carboxylate

Procedure: To a stirred solution of tert-butyl 4-(2-aminoethyl)piperidine-1-carboxylate (3.5 g, 4.38 mmol) in dichloromethane (300 mL) was added (tert-butoxycarbonyl)((4-(dimethyliminio)pyridin-1(4H)-yl)sulfonyl)amide (3.69 g, 12.28 mmol) and diisopropylethylamine (3.96 mL, 23.02 mmol). Reaction mixture was stirred at room temperature for 16 h and monitored by TLC. The reaction mixture was diluted with dichloromethane (50 mL), washed with water (2 × 30 mL) followed by brine (20 mL), dried over anhydrous sodium sulphate, filtered and evaporated under reduced pressure. The crude residue was purified by combiflash using 25-30% ethyl acetate in hexane to afford tert-butyl 4-(2-((N-(tert-butoxycarbonyl)sulfamoyl)amino)ethyl)piperidine-1-carboxylate as white solid (3.4 g, 54% over 2 steps). LC-MS (ES) m/z = 408.3 [M+H]⁺

### Step 4: Synthesis of N-(2-(Piperidin-4-yl)Ethyl)Sulfamide Hydrochloride

Procedure: To a stirred solution of tert-butyl 4-(2-((N-(tert-butoxycarbonyl) sulfamoyl)amino)ethyl)piperidine-1-carboxylate (2.0 g, 4.91 mmol) in 1,4-dioxane (10 mL) was added 4M HCl in 1,4-dioxane (70 mL). Reaction mixture was stirred at room temperature for 6 h and monitored by TLC. The reaction mixture was evaporated under reduced pressure and co-distilled with toluene (twice) and dried to get N-(2-(piperidin-4-yl)ethyl)sulfamide hydrochloride as off-white solid (1.2 g, crude). LC-MS (ES) m/z = 208.1 [M+H]⁺

### Step 5: Synthesis of N-(2-(1-(2-amino-6,7-Dimethoxyquinazolin-4-yl)Piperidin-4-yl)Ethyl)Sulfamide

Procedure: To a stirred solution of 4-chloro-6,7-dimethoxyquinazolin-2-amine (0.1 g, 0.41 mmol) in DMF (6 mL) was added (2-(piperidin-4-yl)ethyl)sulfamide hydrochloride (0.12 g, 0.50 mmol) and solution of potassium carbonate (0.11 g, 0.82 mmol) in water (0.5 mL) and the resulting reaction mixture was stirred at 90° C. for 12 h. The progress of the reaction was monitored by TLC. Reaction mixture was quenched with water (20 mL) and extracted with ethyl acetate (2 × 30 mL). The combined organic layers were washed with brine (30 mL), dried over sodium sulphate, filtered and evaporated under reduced pressure. The crude obtained was purified by combiflash purifier using 5% methanol in dichloromethane to afford N-(2-(1-(2-amino-6,7-dimethoxyquinazolin-4-yl)piperidin-4-yl)ethyl)sulfamide as an off white solid (0.016 g, 9%). ¹H NMR (400 MHz, DMSO-d₆): δ 6.92 (s, 1 H), 6.72 (s, 1 H), 6.43 (s, 2 H), 6.38-6.42 (m, 1 H), 5.93 (bs, 2 H), 3.97 (d, J = 12.8 Hz, 2 H), 3.81 (s, 3 H), 3.77 (s, 3 H), 2.84-2.95 (m, 4 H), 1.76 (d, J= 12 Hz, 2 H), 1.65-1.75 (m, 1 H), 1.47 (q, J= 7.2 Hz, 2H), 1.29-1.40 (m, 2 H). LC-MS (ES) m/z = 411.2 [M+H]⁺; HPLC purity: 99.33%.

Example 2: N-(2-(1-(6,7-dimethoxy-2-(methylamino)quinazolin-4-yl)piperidin-4-yl)ethyl)sulfamide

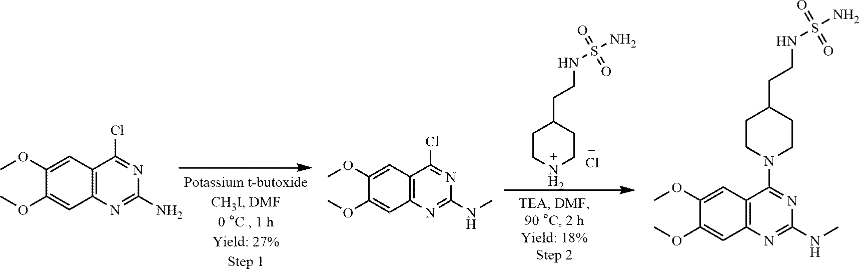

### Step1: Synthesis of 4-Chloro-6,7-Dimethoxy-N-Methylquinazolin-2-Amine

Procedure: To a stirred solution of 4-chloro-6,7-dimethoxyquinazolin-2-amine (0.3 g, 1.25 mmol) in dimethylformamide (5 mL) was added potassium tert-butoxide (0.21 g, 1.87 mmol) at 0° C. and then added methyl iodide (0.1 mL, 1.50 mmol) and stirred the reaction mixture for 1 h. The progress of the reaction was monitored by TLC. The reaction mixture was quenched with ice cold water (100 mL) to the reaction mixture and extracted with ethyl acetate (3 × 100 mL), combined organic layers were washed with brine (50 mL). The organic layer was dried over sodium sulphate, filtered and evaporated under reduced pressure to obtain crude compound. The crude residue was purified by gradient column chromatography using 30% ethyl acetate in hexane to afford 4-chloro-6,7-dimethoxy-N-methylquinazolin-2-amine (0.1 g, 27%) as yellow solid. LC-MS (ES) m/z = 254.1 [M+H]⁺.

### Step 2: Synthesis of N-(2-(1-(6,7-Dimethoxy-2-(Methylamino)Quinazolin-4-yl)Piperidin-4-yl)Ethyl)Sulfamide

Procedure: To a stirred solution of 4-chloro-6,7-dimethoxy-N-methylquinazolin-2-amine (0.10 g, 0.59 mmol) in dimethyl formamide (3 mL) was added triethyl amine (0.23 mL, 1.77 mmol) followed by 4-(2-(sulfamoylamino)ethyl)piperidin-1-ium chloride (0.21 g, 0.88 mmol) and heated to 90° C. for 2 h. The progress of the reaction was monitored by TLC. Then the reaction was diluted with water (100 mL) and extracted with dichloromethane (2 × 50 mL), combined organic layers were washed with brine (50 mL). The organic layer was dried over sodium sulphate, filtered and evaporated under reduced pressure to obtain crude compound. The crude residue was purified by prep-HPLC. Conditions: Column: Intersil ODS 3 V(250 mmx4.6 mmx5 mic), Mobile phase(A): 0.1% Ammonia in water Mobile phase(B): ACN: Flow rate: 1.0 mL/min to afford N-(2-(1-(6,7-dimethoxy-2-(methylamino)quinazolin-4-yl)piperidin-4-yl)ethyl)sulfamide (0.030 g, 18%)as white solid. ¹HNMR (400 MHz, DMSO-d₆): δ 6.93 (s, 1 H), 6.79 (s, 1 H), 6.38-6.46 (m, 4 H), 3.95-3.98 (m, 2 H), 3.83 (s, 3 H), 3.78 (s, 3 H), 2.85-2.95 (m, 4 H), 2.78-2.80 (m, 3 H), 1.75-1.78 (m, 2 H), 1.63 (b, 1 H), 1.46-1.49 (m, 2 H), 1.30-1.38 (m, 2 H); LC-MS (ES) m/z = 425.2 [M+H]⁺; HPLC purity: 99.91% .

Example 3: N-(2-(1-(2-(dimethylamino)-6,7-dimethoxyquinazolin-4-yl)piperidin-4-yl)ethyl)sulfamide

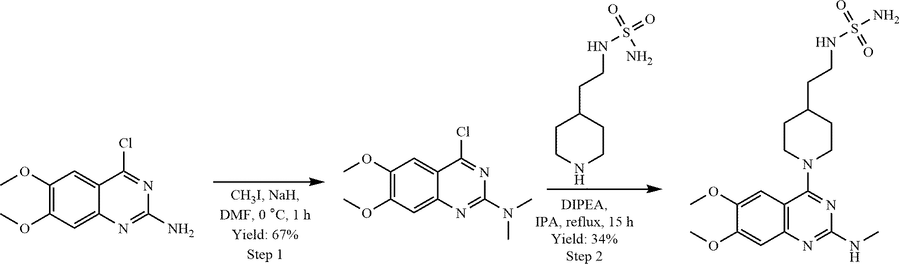

### Step 1: Synthesis of 4-Chloro-6,7-Dimethoxy-N,N-Dimethylquinazolin-2-Amine

Procedure: To a stirred solution of 4-chloro-6,7-dimethoxyquinazolin-2-amine (0.150 g, 0.629 mmol) in DMF (5 mL) was added sodium hydride (0.037 g, 0.93 mmol) portion wise at 0° C. and reaction mixture stirred at same temperature for 30 min. Thereafter iodomethane (0.06 mL, 0.93 mmol) was added and stirring continued at 0° C. for 30 min. The progress of the reaction was monitored by TLC. Reaction mixture was diluted with ice water and extracted using ethyl acetate (3 × 20 mL), dried over sodium sulphate, concentrated. Crude was purified by gradient column chromatography using ethyl acetate in n-hexane to afford 4-chloro-6,7-dimethoxy-N,N-dimethylquinazolin-2-amine as off white solid. (0.150 g, 67%).¹HNMR (400 MHz, DMSO-d₆): δ 7.13 (s, 1 H), 6.94 (s, 1 H), 3.91 (s, 3 H), 3.85 (s, 3 H), 3.14 (s, 6 H). LC-MS (ES) m/z = 268.0 [M+H]⁺

### Step 2: Synthesis of N-(2-(1-(2-(Dimethylamino)-6,7-Dimethoxyquinazolin-4-yl)Piperidin-4-yl)Ethyl)Sulfamide

Procedure: To a stirred solution of 4-chloro-6,7-dimethoxy-N,N-dimethylquinazolin-2-amine (0.1 g, 0.373 mmol) and N-(2-(piperidin-4-yl)ethyl)sulfamide hydrochloride (0.118 g, 0.485 mmol) in isopropyl alcohol (3 mL) was added diisopropyl ethylamine (0.32 mL, 1.864 mmol) and solution heated to reflux for 15 h. The progress of the reaction was monitored by TLC. Solvent was concentrated and residue diluted with water. Organic compound extracted using ethyl acetate (3 × 20 mL), dried over sodium sulphate, concentrated. Crude was purified by gradient column chromatography using methanol in dichloromethane to afford N-(2-(1-(2-(dimethylamino)-6,7-dimethoxyquinazolin-4-yl)piperidin-4-yl)ethyl)sulfamide as an off white solid (0.055 g, 33.74%). ¹HNMR (400 MHz, DMSO-d₆): δ 6.94 (s, 1 H), 6.82 (s, 1 H), 6.43 - 6.41 (m, 3 H), 4.06 - 4.03 (m, 2 H), 3.84 (s, 3 H), 3.78 (s, 3 H), 3.28 (s, 6 H), 2.95 - 2.92 (m, 4 H), 1.78 - 1.75 (m, 2 H), 1.65 (m, 1 H), 1.49 - 1.44 (m, 2 H), 1.38 - 1.29 (m, 2 H). LC-MS (ES) m/z = 439.4 [M+H]⁺. HPLC purity 99.6%.

Example 4: Synthesis of N-(2-(1-(6,7-dimethoxy-2-(pyridin-2-yl)quinazolin-4-yl)piperidin-4-yl)ethyl)sulfamide

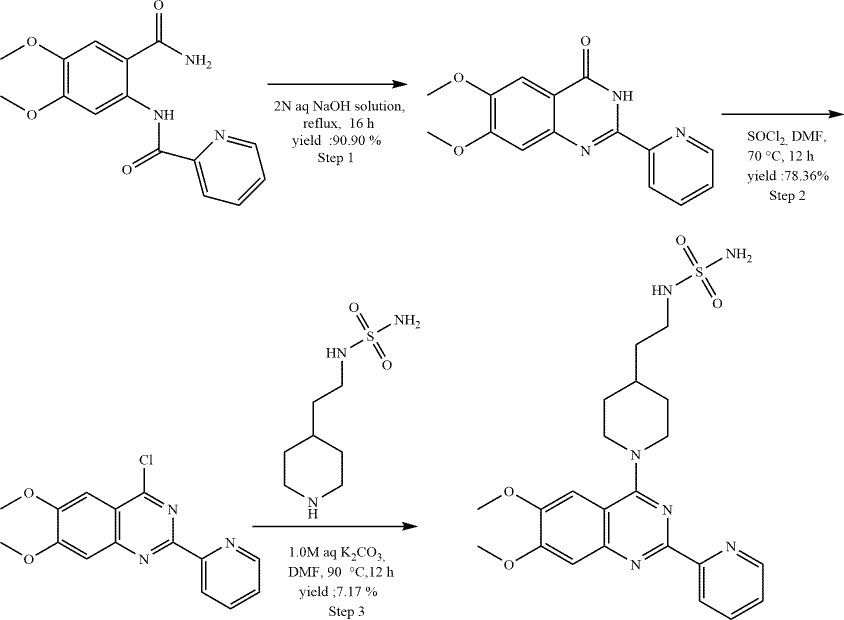

### Step 1: Synthesis of 6,7-Dimethoxy-2-(Pyridin-2-yl)Quinazolin-4(3H)-One

Procedure : To a stirred solution of N-(2-carbamoyl-4,5-dimethoxyphenyl)picolinamide (0.7 g, 2.990 mmol) was added 2N NaOH solution (20 mL, 6.976 mmol). Reaction mixture was stirred for 16 h at 80° C. Progress of the reaction was monitored by TLC. Reaction mixture was diluted with water (20 mL), reaction mixture was acidified with 1N HCl solution pH maintained at 7, extracted with ethyl acetate (2 × 50 mL). The combined organic layer was washed with brine (10 mL), dried over anhydrous sodium sulphate, filtered and evaporated under reduced pressure to afford 6,7-dimethoxy-2-(pyridin-2-yl)quinazolin-4(3H)-one as white solid, (0.6 g, 90.90%). ¹HNMR (400 MHz, DMSO-d₆): δ 11.58(s, 1 H),8.72 (d, J= 5.2 Hz, 1 H),8.40 (d, J= 8.0 Hz, 1 H),8.03-8.07 (m, 1 H),7.60-7.63 (m, 1 H),7.50 (s, 1 H),7.26 (s, 1 H),3.94 (s, 3 H),3.89 (s, 3 H).LC-MS (ES) m/z = 284.1[M+H]⁺

### Step 2: Synthesis of 4-Chloro-6,7-Dimethoxy-2-(Pyridin-2-yl)Quinazoline

Procedure: To a stirred solution of 6,7-dimethoxy-2-(pyridin-2-yl)quinazolin-4(3H)-one (0.6 g, 2.120 mmol) in DMF (10 mL) was added SOCl₂ (6 mL, 75.63 mmol). Reaction mixture was stirred at 80° C. for 2 h, reaction was monitored by TLC. The reaction mixture was diluted with ethyl acetate (30 mL), washed with water (2 × 10 mL) followed by brine (10 mL), dried over anhydrous sodium sulphate, filtered and evaporated under reduced pressure. The crude residue was to afford 4-chloro-6,7-dimethoxy-2-(pyridin-2-yl)quinazoline as white solid (0.5 g, 78.36%). ¹HNMR (400 MHz, CDCl₃)— δ 8.88 (d, J = 4 Hz, 1 H), 8.62 (d, J = 8.0 Hz, 1 H),7.88 (d, J= 7.2 Hz, 1H),7.69 (s, 1 H),7.41-7.45 (m, 2 H), 4.02-4.09 (m, 6 H). LC-MS (ES) m/z = 302.2 [M+H]⁺

### Step 3: Synthesis of N-(2-(1-(6,7-Dimethoxy-2-(Pyridin-2-yl)Quinazolin-4-yl)Piperidin-4-yl)Ethyl)Sulfamide

Procedure: To a stirred solution of 4-chloro-6,7-dimethoxy-2-(pyridin-2-yl)quinazoline (0.4 g, 1.328 mmol) in DMF (10 mL) were added N-(2-(piperidin-4-yl)ethyl)sulfamide hydrochloride (0.3 g, 1.449 mmol), 1 M K2CO3 in H₂O (0.366 g, 2.652 mmol). Reaction mixture was stirred at 90° C. for 16 h, reaction was monitored by TLC. The reaction mixture was diluted with DCM (30 mL), washed with water (2 × 10 mL) followed by brine (10 mL), dried over anhydrous sodium sulphate, filtered and evaporated under reduced pressure. The crude residue was purified by combiflash using 4.5% methanol in dichloromethane to afford N-(2-(1-(6,7-dimethoxy-2-(pyridin-2-yl)quinazolin-4-yl)piperidin-4-yl)ethyl)sulfamide as off-white solid (0.045 g, 7.17%). ¹HNMR (400 MHz, DMSO-d₆): δ 8.89 (d, J = 4.4 Hz, 1 H), 8.58 (d, J = 8.0 Hz, 1 H), 8.17 (t, J = 7.6 Hz, 1 H), 7.83 (s, 1 H), 7.78 (t, J = 5.6 Hz, 1 H), 7.35 (s, 1 H), 6.45 (s, 3 H), 4.82 (d, J = 12.4 Hz, 2 H), 3.97 (d, J = 4.8 Hz, 6 H), 3.51 (d, J = 11.2 Hz, 3 H), 2.95 (d, J = 5.2 Hz, 2 H), 1.94 (d, J = 12.8 Hz, 3 H), 1.38-1.48 (m, 4 H), 1.22 (s, 1H).HPLC purity 98.89%. LC-MS (ES) m/z = 473.2 [M+H]⁺

Example 5: N-(2-(1-(6,7-dimethoxy-2-(pyridin-3-yl)quinazolin-4-yl)piperidin-4-yl)ethyl)sulfamide

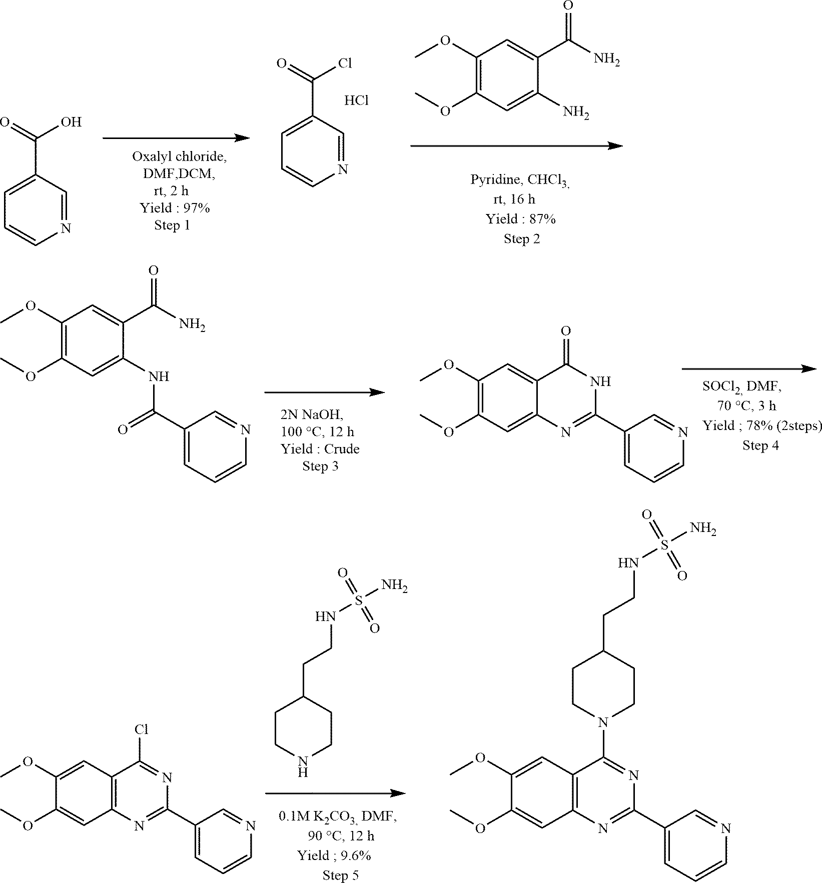

### Step 1: Synthesis of Nicotinoyl Chloride Hydrochloride

Procedure: To a stirred solution of nicotinic acid (1.0 g, 8.12 mmol) in dry dichloromethane (20 mL) was added oxalyl chloride (1.05 mL, 12.18 mmol) and 5 drops of DMF at room temperature and the resulting reaction mixture was stirred for 2 h at room temperature. After completion of the reaction monitored by TLC, reaction mixture was evaporated under reduced pressure. The residue obtained was dissolved in toluene and again concentrated under vacuum and carry forwarded to next step.

### Step 2: Synthesis of N-(2-Carbamoyl-4,5-Dimethoxyphenyl)Nicotinamide

Procedure: To a stirred solution of 2-amino-4,5-dimethoxybenzamide (0.3 g, 1.52 mmol) in CHCl₃ (10 mL), pyridine (0.366 mL, 4.56 mmol) was added at RT and the mixture was stirred for 10 mins, nicotinoyl chloride hydrochloride (0.285 g, 1.6 mmol) was added at room temperature. The reaction mixture was stirred at RT for 16 h. The progress of the reaction was monitored by TLC. Then the reaction was quenched with cold water (10 mL) and extracted with dichloromethane (3 × 50 mL), combined organic layers were washed with aqueous 10% citric acid solution (10 mL) and brine (10 mL). The organic layer was dried over anhydrous sodium sulphate, filtered and evaporated under reduced pressure, purified by combiflash purifier using ethyl acetate in n-hexane to afford N-(2-carbamoyl-4,5-dimethoxyphenyl)nicotinamide (0.4 g, 87%) as an off white solid. LC-MS (ES) m/z = 302.1 [M+H]⁺

### Step 3: Synthesis of 6,7-Dimethoxy-2-(Pyridin-3-yl)Quinazolin-4(3H)-One

Procedure: To a stirred solution of N-(2-carbamoyl-4,5-dimethoxyphenyl)nicotinamide (0.4 g, 1.328 mmol) was added 2N NaOH solution (8 mL). Reaction mixture was stirred for 12 h at 100° C. Progress of the reaction was monitored by TLC. Cooled to room temperature, extracted with ethyl acetate (2 × 50 mL). The combined organic layer was washed with brine (10 mL), dried over anhydrous sodium sulphate, filtered and evaporated under reduced pressure. The crude residue was purified by combiflash using 0.5-10% methanol in dichloromethane to afford 6,7-dimethoxy-2-(pyridin-3-yl)quinazolin-4(3H)-one as white solid, (0.4 g). LC-MS (ES) m/z = 284.1[M+H]⁺

### Step 4: Synthesis of 4-Chloro-6,7-Dimethoxy-2-(Pyridin-3-yl)Quinazoline

Procedure: To a stirred solution of SOCl₂(1.02 mL, 14.11 mmol), 6,7-dimethoxy-2-(pyridin-3-yl)quinazolin-4(3H)-one (0.4 g, 1.41 mmol) was added at 0° C. followed by catalytic amount of DMF (0.02 mL) was added Reaction mixture was stirred at 80° C. for 3 h, reaction was monitored by TLC. The reaction mixture was evaporated under reduced pressure. The crude was neutralized with 1N NaOH up to PH~7 and then extracted with DCM (30 mL), washed with water (2 × 10 mL) followed by brine (10 mL), dried over anhydrous sodium sulphate, filtered and evaporated under reduced pressure. The crude residue was purified by combiflash purified using 5-50% ethyl acetate in n-hexane to afford 4-chloro-6,7-dimethoxy-2-(pyridin-3-yl)quinazoline as an off-white solid (0.335 g, 78%). LC-MS (ES) m/z = 302.1 [M+H]⁺

### Step 5: Synthesis of N-(2-(1-(6,7-Dimethoxy-2-(Pyridin-3-yl)Quinazolin-4-yl)Piperidin-4-yl)Ethyl)Sulfamide

Procedure: To a stirred solution of 4-chloro-6,7-dimethoxy-2-(pyridin-3-yl)quinazoline (0.1 g, 10.33 mmol) in DMF (10 mL) were added N-(2-(piperidin-4-yl)ethyl)sulfamide hydrochloride (0.1 g, 0.364 mmol), 0.1 M K₂CO₃ in H₂O (8.25 mL, 0.825 mmol). Reaction mixture was stirred at 90° C. for 12 h, reaction was monitored by TLC. The reaction mixture was diluted with DCM (30 mL), washed with water (2 × 10 mL) followed by brine (10 mL), dried over anhydrous sodium sulphate, filtered and evaporated under reduced pressure. The crude residue was purified by combiflash using 4.5% methanol in dichloromethane to afford -(2-(1-(6,7-dimethoxy-2-(pyridin-3-yl)quinazolin-4-yl)piperidin-4-yl)ethyl)sulfamide as an off white solid (0.015 g, 9.6%). ¹HNMR (400 MHz, DMSO-d₆): 89.55 (s, 1 H), 8.685 (d, J = 7.8 Hz, 1 H), 8.63 (d, J = 3.6 Hz, 1 H), 7.52-7.49 (m, 1 H), 7.29 (s, 1 H), 7.15 (s, 1 H), 6.44-6.41(m, 3 H), 4.3 (d, J= 12.8 Hz, 2 H), 3.95 (s, 6 H), 3.15 (t, J= 12.0 Hz, 2 H), 2.968 (q, J= 6.0 Hz, 2 H), 1.84 (d, J= 12.4 Hz, 2 H), 1.73(m, 1 H), 1.489 (q, J = 6.8 Hz, 2 H), 1.39 (d, J= 10.8 Hz, 2 H). LC-MS (ES) m/z = 473[M+H]⁺. HPLC purity 99.46%.

Example 6: Synthesis of N-(2-(1-(6,7-dimethoxy-2-(pyridin-4-yl)quinazolin-4-yl)piperidin-4-yl)ethyl)sulfamide

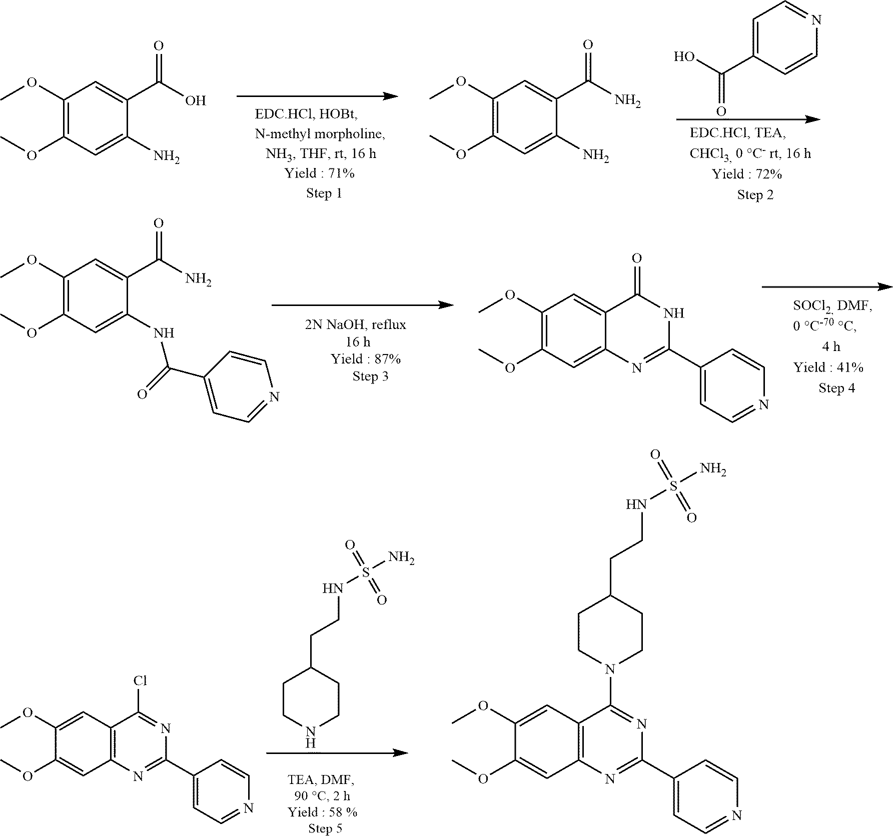

### Step1: Synthesis of 2-Amino-4,5-Dimethoxybenzamide

Procedure: To a stirred solution of 2-amino-4,5-dimethoxybenzoic acid (10 g, 50.71 mmol) in tetrahydrofuran (100 mL) was added EDC.HCl (19.44 g, 101.42 mmol), HOBt (13.70 g, 101.42 mmol) and N-methyl morpholine (10.25 g, 101.42 mmol) in dropwise at 0° C., then added ammonia (15 mL) the reaction mixture was stirred at room temperature for 16 h. The progress of the reaction was monitored by TLC. Then the reaction was quenched with ice cold water (100 mL) and extracted with ethyl acetate (3 × 100 mL), combined organic layers were washed with brine (50 mL). The organic layer was dried over sodium sulphate, filtered and evaporated under reduced pressure to obtain crude compound. The crude residue was purified by gradient column chromatography using 70% ethyl acetate in hexane to afford 2-amino-4,5-dimethoxybenzamide (7.1 g, 71%) as Yellow solid. LC-MS (ES) m/z = 197.1 [M+H]⁺.

### Step 2: Synthesis of N-(2-Carbamoyl-4,5-Dimethoxyphenyl)Isonicotinamide

Procedure: To a stirred solution of isonicotinic acid (0.94 g, 7.64 mmol) in chloroform (20 mL) was added EDC.HCl (1.17 g, 6.11 mmol) in portion wise at 0° C. stirred the reaction mixture for 45 mins and then added 2-amino-4,5-dimethoxybenzamide (1 g, 5.09 mmol) and triethylamine (1.78 mL, 12.74 mmol) and allowed the reaction mixture was stirred at room temperature for 16 h. The progress of the reaction was monitored by TLC. Then the reaction was washed with 5% HCl (60 mL) followed by saturated NaHCO₃solution (60 mL) and water (80 mL). The organic layer was dried over sodium sulphate, filtered and evaporated under reduced pressure to obtain N-(2-carbamoyl-4,5-dimethoxyphenyl)isonicotinamide (1.1 g, 72%) as yellow solid as crude compound. The crude compound was carried out for next step without purification.

### Step 3: Synthesis of 6,7-Dimethoxy-2-(Pyridin-4-yl)Quinazolin-4(3H)-One

Procedure: To a stirred solution of N-(2-carbamoyl-4,5-dimethoxyphenyl)isonicotinamide(1.1 g, 3.65 mmol) in 2N Sodium hydroxide (15 mL) was refluxed into 70° C. for 16 h. The progress of the reaction was monitored by TLC. The reaction mixture was acidified using 1N HCl to adjust the pH~ 2 and extracted with ethyl acetate (60 mL). The organic layer was dried over sodium sulphate, filtered and evaporated under reduced pressure to obtain 6,7-dimethoxy-2-(pyridin-4-yl)quinazolin-4(3H)-one (0.9 g, 87%) as off-white solid as crude compound. LC-MS (ES) m/z = 284.1 [M+H]⁺. The crude compound was carried out for next step without purification.

### Step 4: Synthesis of 4-Chloro-6,7-Dimethoxy-2-(Pyridin-4-yl)Quinazoline

Procedure: To a stirred solution of 6,7-dimethoxy-2-(pyridin-4-yl)quinazolin-4(3H)-one (0.5 g, 1.76 mmol) in dimethylformamide (0.8 mL) was added thionyl chloride (2 mL) at 0° C. and refluxed the reaction mixture for 4 h. The progress of the reaction was monitored by TLC. Evaporated the solvent under reduced pressure and residue was quenched with sat NaHCO₃ and extracted with dichloromethane (100 mL) was dried over sodium sulphate, filtered and evaporated under reduced pressure to obtain crude compound. The crude residue was purified by gradient column chromatography using 30% ethyl acetate in hexane to afford 4-chloro-6,7-dimethoxy-2-(pyridin-4-yl)quinazoline (0.22 g, 41%) as off-white solid. LC-MS (ES) m/z = 302.1 [M+H]⁺.

### Step 5: Synthesis of N-(2-(1-(6,7-Dimethoxy-2-(Pyridin-4-yl)Quinazolin-4-yl)Piperidin-4-yl)Ethyl)Sulfamide

Procedure: To a stirred solution of 4-chloro-6,7-dimethoxy-2-(pyridin-4-yl)quinazoline (0.15 g, 0.49 mmol) in dimethyl formamide (6 mL) was added and 4-(2-(sulfamoylamino)ethyl)piperidin-1-ium chloride (0.18 g, 0.74 mmol) was added 1 M aqueous potassium carbonate (0.2 mL, 1.49 mmol) and heated the reaction mixture to 90° C. for 2 h. Progress of the reaction was monitored by TLC. The reaction mixture was diluted with water (20 mL), extracted with dichloromethane (2 × 40 mL). The combined organic layer ware washed with brine (20 mL), dried over anhydrous sodium sulphate, filtered and evaporated under reduced pressure to obtain crude compound. The crude residue was purified by gradient column chromatography using 90% ethyl acetate in hexane to afford N-(2-(1-(6,7-dimethoxy-2-(pyridin-4-yl)quinazolin-4-yl)piperidin-4-yl)ethyl)sulfamide (0.18 g, 58%) as white solid. ¹HNMR (400 MHz, DMSO-d₆): δ 8.70-8.72 (m, 2 H), 8.29-8.30 (m, 2 H), 7.31 (s, 1 H), 7.16 (s, 1 H), 6.43-6.45 (m, 3 H), 4.30-4.33 (m, 2 H), 3.95 (s, 3 H), 3.92 (s, 3 H), 3.17 (t, J = 12 Hz, 2 H), 2.94-2.96 (m, 2 H), 1.83-1.86 (m, 2 H), 1.74 (b, 1 H), 1.48-1.50 (m, 2 H), 1.32-1.38 (m, 2 H). LC-MS (ES) m/z = 473.2 [M+H]⁺. HPLC purity: 99.73%..

Example 7: N-(4-(4-(2-(sulfamoylamino)ethyl)piperidin-1-yl)pyrimidin-2-yl)nicotinamide

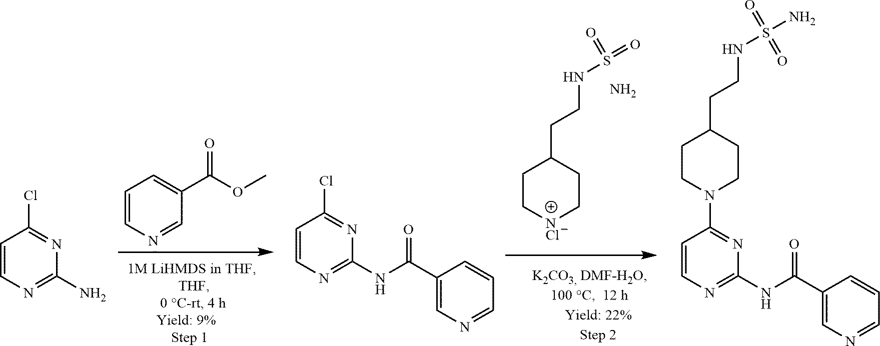

### Step 1: Synthesis of N-(4-Chloropyrimidin-2-yl)Nicotinamide

Procedure: To a stirred solution of 2-amino-4-chloropyrimidine (0.5 g, 0.38 mmol) in THF (25 mL) was added 1M LiHMDS solution in THF (7.7 mL, 0.77 mmol) at 0° C. and the resulting reaction mixture was stirred for 30 minutes at room temperature followed by added solution of methyl nicotinate (0.10 g, 0.77 mmol) in THF and the reaction mixture was stirred for 4 h at room temperature. After the completion of the reaction, monitored by TLC using 5% methanol in DCM as eluent, reaction mixture was quenched with saturated aqueous solution of ammonium chloride and extracted with ethyl acetate (2*50 mL) and separated the organic layer. The combined organic layer was dried over anhydrous dried over anhydrous sodium sulphate, filtered and evaporated under reduced pressure. The crude obtained was purified by combiflash purifier using 5% methanol in DCM as eluent to afford N-(4-chloropyrimidin-2-yl)nicotinamide (0.08 g , 9%). ¹HNMR (400 MHz, DMSO-d₆): 8 11.5 (s, 1 H), 9.05-9.06 (m, 1 H), 8.74-8.75 (m, 1 H), 8.69 (d, J = 5.2 Hz, 1 H), 8.26 (d, J= 8 Hz, 1 H), 7.51-7.54 (m, 1 H), 7.43 (d, J = 5.2 Hz, 1 H). LC-MS (ES) m/z = 235.1 [M+H]⁺.

### Step 2: Synthesis of N-(4-(4-(2-(Sulfamoylamino)Ethyl)Piperidin-1-yl)Pyrimidin-2-yl)Nicotinamide

Procedure: To a stirred solution of N-(4-chloropyrimidin-2-yl)nicotinamide (0.08 g, 0.34 mmol) in DMF (4 mL) was added (2-(piperidin-4-yl)ethyl)sulfamide hydrochloride (0.124 g, 0.51 mmol) and aqueous solution of potassium carbonate (0.14 g, 1.02 mmol) and the resulting reaction mixture was stirred at 100° C. for 12 h. The progress of the reaction was monitored by TLC using 5% MeOH-DCM as eluent. After the completion of the reaction, quenched with water (20 mL) and extracted with ethyl acetate (2 × 30 mL). The combined organic layers were washed with brine (30 mL), dried over sodium sulphate, filtered and evaporated under reduced pressure. The crude obtained was purified by combiflash purifier using 5% MeOH-DCM as eluent to afford N-(4-(4-(2-(sulfamoylamino)ethyl)piperidin-1-yl)pyrimidin-2-yl)nicotinamide as pale yellow solid (0.003 g, 22%). ¹HNMR (400 MHz, DMSO-d₆): δ 10.59 (s, 1 H), 8.95 (s, 1 H), 8.68-8.69 (m, 1 H), 8.15-8.20 (m, 1 H), 8.03-8.05 (m, 1 H), 7.47-7.50 (m, 1 H), 6.53 (d, J = 6 Hz, 1 H), 6.43 (m, 2 H), 6.39 (m, 1 H), 4.25-4.35 (m, 2 H), 2.87-2.92 (m, 2 H), 2.79 (t, J = 12.4 Hz, 2 H), 1.60-1.75 (m, 3 H), 1.35-1.45 (m, 2 H), 0.95-1.05 (m, 2 H). LC-MS (ES) m/z = 406.2 [M+H]⁺.

Example 8: N-(2-(1-(6,7-dimethoxy-2-phenylquinazolin-4-yl)piperidin-4-yl)ethyl)sulfamide

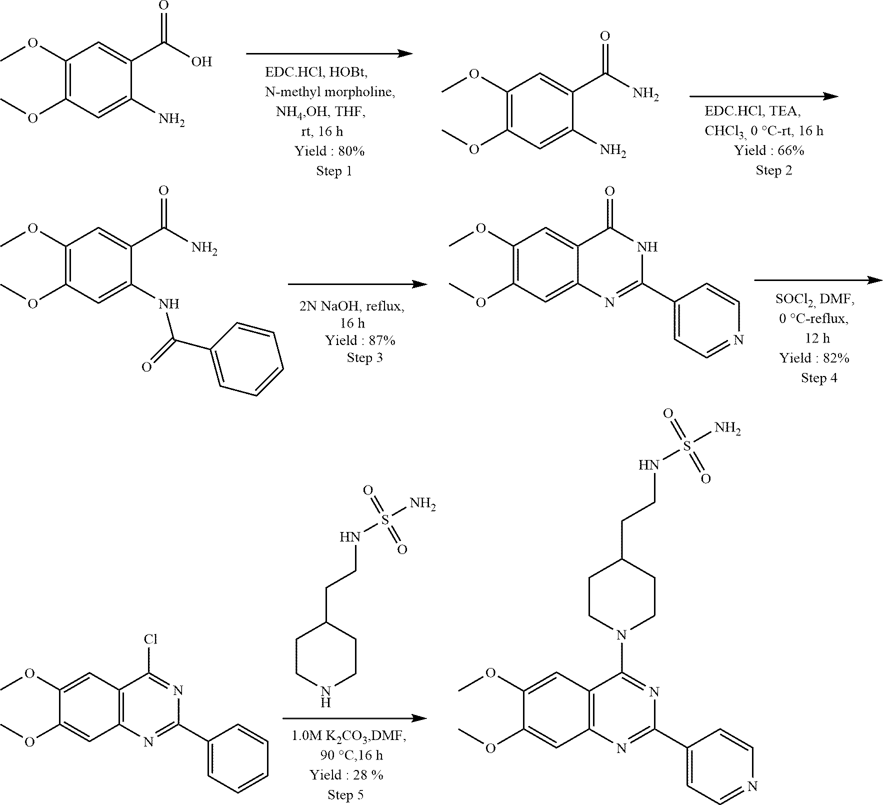

### Step 1: Synthesis of 2-Amino-4,5-Dimethoxybenzamide

Procedure: To a stirred solution of 2-amino-4,5-dimethoxybenzoic acid (5 g, 25.35 mmol) in tetrahydrofuran (100 mL) was added EDC.HCl (9.72 g, 50.71 mmol), HOBt (6.85 g, 50.71 mmol) and N-Methyl morpholine (5.13 g, 50.71 mmol) in dropwise at 0° C., then added ammonium hydroxide (5.58 mL) the reaction mixture was stirred at room temperature for 16 h. The progress of the reaction was monitored by TLC. Then the reaction was quenched with ice cold water (100 mL) and extracted with ethyl acetate (3 × 100 mL), combined organic layers were washed with brine (50 mL). The organic layer was dried over sodium sulphate, filtered and evaporated under reduced pressure to obtain crude compound. The crude residue was purified by gradient column chromatography using 70% ethyl acetate in hexane to afford 2-amino-4,5-dimethoxybenzamide(4 g, 80%) as Yellow solid. LC-MS (ES) m/z = 197.1 [M+H]⁺.

### Step 2: Synthesis of 2-Benzamido-4,5-Dimethoxybenzamide

Procedure: To a stirred solution of benzoic acid (0.93 g, 7.64 mmol) in chloroform (20 mL) was added EDC.HCl (1.17 g, 6.11 mmol) in portion wise at 0° C. stirred the reaction mixture for 45 mins and then added 2-amino-4,5-dimethoxybenzamide (1 g, 5.09 mmol) and triethylamine (1.78 mL, 12.74 mmol) and allowed the reaction mixture was stirred at room temperature for 16 h. The progress of the reaction was monitored by TLC. Then the reaction was washed with 5% HCl (60 mL) followed by sat NaHCO₃ (60 mL) and water (80 mL). The organic layer was dried over sodium sulphate, filtered and evaporated under reduced pressure to obtain2-benzamido-4,5-dimethoxybenzamide (1 g, 66%) as off-white solid as crude compound. The crude compound was carried out for next step without purification.

### Step 3: Synthesis of 6,7-Dimethoxy-2-Phenylquinazolin-4(3H)-One

Procedure: To a stirred solution of 2-benzamido-4,5-dimethoxybenzamide (1 g, 3.32 mmol) in 2N Sodium hydroxide (15 mL) was refluxed into 70° C. for 16 h. The progress of the reaction was monitored by TLC. The reaction mixture was acidified using 1N HCl to adjust the pH~ 2 and extracted with ethyl acetate (60 mL). The organic layer was dried over sodium sulphate, filtered and evaporated under reduced pressure to obtain6,7-dimethoxy-2-phenylquinazolin-4(3H)-one (0.82 g, 87%) as off white solid as crude compound. LC-MS (ES) m/z = 283.1 [M+H]⁺. The crude compound was carried out for next step without purification.

### Step 4: Synthesis of 4-Chloro-6,7-Dimethoxy-2-Phenylquinazoline

Procedure: To a stirred solution of 6,7-dimethoxy-2-phenylquinazolin-4(3H)-one (0.3 g, 1.06 mmol) in dimethylformamide (0.5 mL) was added thionyl chloride (1 mL) at 0° C. and refluxed the reaction mixture for 4 h. The progress of the reaction was monitored by TLC. Evaporated the solvent under reduced pressure and residue was quenched with sat NaHCO₃ and extracted with dichloromethane (100 mL) was dried over sodium sulphate, filtered and evaporated under reduced pressure to obtain the crude compound. The crude residue was purified by gradient column chromatography using 12% ethyl acetate in hexane to afford 4-chloro-6,7-dimethoxy-2-phenylquinazoline (0.26 g, 82%) as off white solid. LC-MS (ES) m/z = 301.2 [M+H]⁺.

### Step 5: Synthesis of N-(2-(1-(6,7-Dimethoxy-2-Phenylquinazolin-4-yl)Piperidin-4-yl)Ethyl)Sulfamide

Procedure: To a stirred solution of 4-chloro-6,7-dimethoxy-2-phenylquinazoline (0.15 g, 0.49 mmol) in dimethyl formamide (4 mL) was added and 4-(2-(sulfamoylamino)ethyl)piperidin-1-ium chloride (0.15 g, 0.74 mmol) was added 1 M aq potassium carbonate (0.13 g, 0.99 mmol) and heated the reaction mixture to 90° C. for 16 h. Progress of the reaction was monitored by TLC. The reaction mixture was diluted with water (20 mL), extracted with dichloromethane (2 × 40 mL). The combined organic layer ware washed with brine (20 mL), dried over anhydrous sodium sulphate, filtered and evaporated under reduced pressure to obtain the crude compound. The crude residue was purified by gradient column chromatography using 80% ethyl acetate in hexane to afford N-(2-(1-(6,7-dimethoxy-2-phenylquinazolin-4-yl)piperidin-4-yl)ethyl)sulfamide (0.065 g, 28%) as white solid. ¹HNMR (400 MHz, DMSO-d₆): δ 8.43-8.45 (m, 2 H), 7.44-7.49 (m, 3 H), 7.26 (s, 1 H), 7.13 (s, 1 H), 6.41-6.44 (m, 3 H), 4.24-4.28 (m, 2 H), 3.94 (s, 3 H), 3.90 (s, 3 H), 3.08-3.15 (m, 2 H), 2.92-2.97 (m, 2 H), 1.82-1.85 (m, 2 H), 1.72 (b, 1 H), 1.46-1.51 (m, 2 H), 1.36-1.41 (m, 2 H).LC-MS (ES) m/z = 472.2 [M+H]⁺. HPLC purity 99.64%.

Example 9: N-(2-(1-(6-(benzo[d]oxazol-6-yl)pyrimidin-4-yl)piperidin-4-yl)ethyl)sulfamide

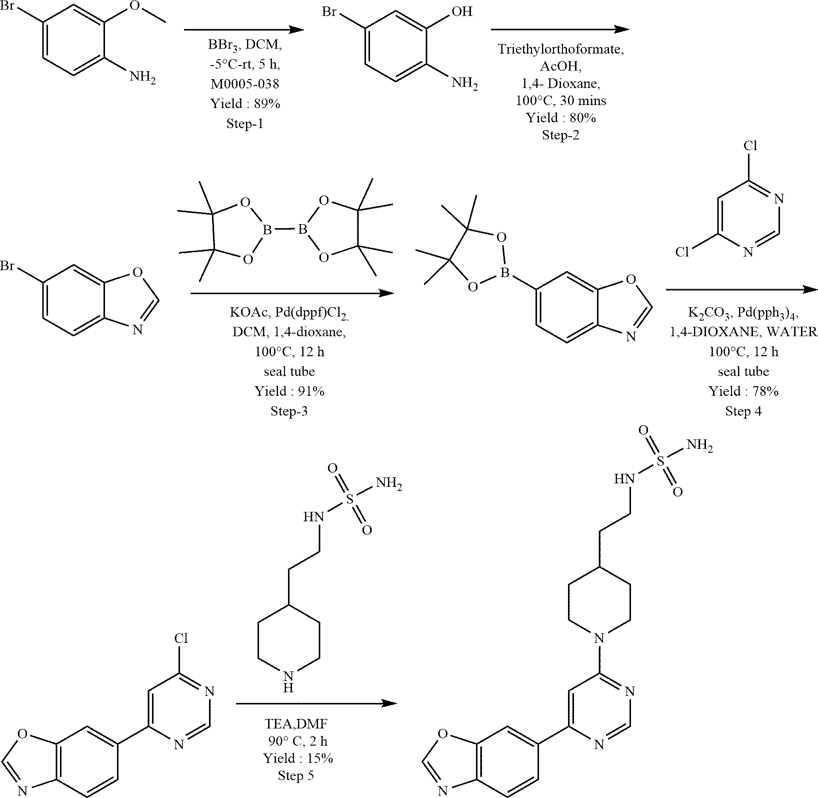

### Step 4: Synthesis of 6-(6-Chloropyrimidin-4-yl)Benzo[d]Oxazole

Procedure: To a stirred solution of 4,6-dichloropyrimidine (0.5 g, 3.35 mmol) in 1,4-dioxane (5 mL) was added 6-(4,4,5,5-tetramethyl-1,3,2-dioxaborolan-2-yl)benzo[d]oxazole (0.82 g, 3.35 mmol) the reaction mixture was purged with argon was added potassium carbonate (1.39 g, 10.06 mmol) followed by Pd(PPh₃)₄ (0.19 g, 0.16 mmol) and heated in a sealed tube at 100° C. for 12 h. The progress of the reaction was monitored by TLC. Added water (100 mL) to the reaction mixture and extracted with ethyl acetate (3 × 100 mL), combined organic layers were washed with brine (50 mL). The organic layer was dried over sodium sulphate, filtered and evaporated under reduced pressure to obtain the crude compound. The crude residue was purified by gradient column chromatography using 15% ethyl acetate in hexane to afford 6-(6-chloropyrimidin-4-yl)benzo[d]oxazole (0.61 g, 78%) as off white solid. LC-MS (ES) m/z = 232.0 [M+H]⁺.

### Step 5: Synthesis of N-(2-(1-(6-(Benzo[d]Oxazol-6-yl)Pyrimidin-4-yl)Piperidin-4-yl)Ethyl)Sulfamide

Procedure: To a stirred solution of 6-(6-chloropyrimidin-4-yl)benzo[d]oxazole (0.15 g, 0.64 mmol) in dimethyl formamide (5 mL) was added triethyl amine (0.27 mL, 1.94 mmol) followed by 4-(2-(sulfamoylamino)ethyl)piperidin-1-ium chloride (0.23 g, 0.97 mmol) and heated to 90 °C2 h. The progress of the reaction was monitored by TLC. Then the reaction was diluted with water (100 mL) and extracted with dichloromethane (2 × 50 mL), combined organic layers were washed with brine (50 mL). The organic layer was dried over sodium sulphate, filtered and evaporated under reduced pressure to obtain crude compound. The crude residue was purified by gradient column chromatography using 5% methanol in dichloromethane to afford N-(2-(1-(6-(benzo[d]oxazol-6-yl)pyrimidin-4-yl)piperidin-4-yl)ethyl)sulfamide (0.04 g, 15%) as white solid. ¹HNMR (400 MHz, DMSO-d₆): δ 8.81 (s, 1 H), 8.55 (s, 2 H), 8.25 (d, J= 8 Hz, 1 H), 7.86 (d, J= 8.4 Hz, 1 H), 7.40 (s, 1 H), 6.39-6.43 (m, 3 H), 4.55-4.58 (m, 2 H), 2.89-2.94 (m, 4 H), 1.73-1.76 (m, 3 H), 1.41-1.42 (m, 2 H), 1.07-1.07 (m, 2 H); LC-MS (ES) m/z =403.1 [M+H]⁺; HPLC purity: 99.73%.

Example 10: Synthesis of N-(2-(1-(6-(benzo[d][1,3]dioxol-5-yl)pyrimidin-4-yl)piperidin-4-yl)ethyl)sulfamide

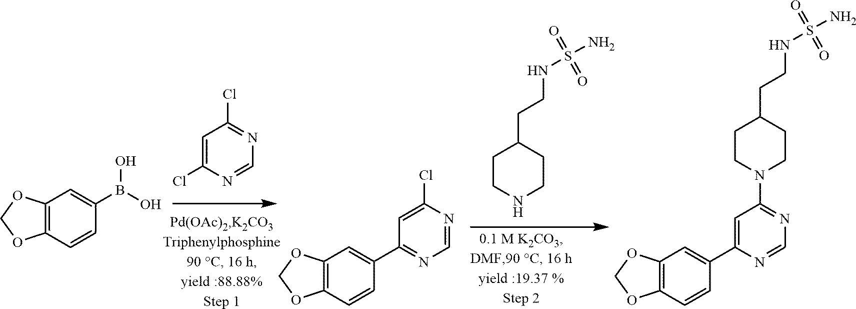

### Step 1: Synthesis of 4-(Benzo[d][1,3]Dioxol-5-yl)-6-Chloropyrimidine

Procedure: To a stirred solution of benzo[d][1,3]dioxol-5-ylboronic acid (1 g, 6.026 mmol) and 4,6-dichloropyrimidine (0.94 g, 6.309 mmol) in ethylene glycol dimethylether/ water (10 mL) were added K₂CO₃ (0.7 g, 5.072 mmol) and Pd(OAc)₂ (0.067 g, 0.299 mmol), followed by triphenylphosphine (1.24 g, 4.732 mmol). Reaction mixture was stirred for 16 h at 90° C. Progress of the reaction was monitored by TLC. Reaction mixture was diluted with water (20 mL), extracted with ethyl acetate (2 × 50 mL). The combined organic layer was washed with brine (10 mL), dried over anhydrous sodium sulphate, filtered and evaporated under reduced pressure. The crude residue was purified by combiflash using ethyl acetate in hexane to afford 4-(benzo[d][1,3]dioxol-5-yl)-6-chloropyrimidine as white solid, (0.8 g, 88.88%). ¹HNMR (400 MHz, CDCl₃): δ 8.95(s, 1H),7.63 (t, J = 5.6 Hz, 3 H), 6.92 (d, J = 8.4 Hz, 1 H),6.06 (s, 2 H). LC-MS (ES) m/z = 235.1 [M+H]⁺

### Step 2: Synthesis of N-(2-(1-(6-(Benzo[d][1,3]Dioxol-5-yl)Pyrimidin-4-yl)Piperidin-4-yl)Ethyl)Sulfamide

Procedure: To a stirred solution of 4-(benzo[d][1,3]dioxol-5-yl)-6-chloropyrimidine (0.15 g, 0.638 mmol) in DMF (10 mL) were added N-(2-(piperidin-4-yl)ethyl)sulfamide hydrochloride (0.158 g, 0.763 mmol), 0.1 M K₂CO₃ in H₂O (12.7 mL, 1.275 mmol). Reaction mixture was stirred at 90° C. for 16 h, reaction was monitored by TLC. The reaction mixture was diluted with ethyl acetate (30mL), washed with water (2 × 10 L) followed by brine (10 mL), dried over anhydrous sodium sulphate, filtered and evaporated under reduced pressure. The crude residue was purified by combiflash using 2.5% methanol in dichloromethane to afford N-(2-(1-(6-(benzo[d][1,3]dioxol-5-yl)pyrimidin-4-yl)piperidin-4-yl)ethyl)sulfamide as off-white solid (0.05 g, 19.37 %). ¹HNMR (400 MHz, DMSO-d₆): δ 8.46 (s, 1 H),7.73 (d, J = 8.4 Hz, 2 H), 7.19 (s, 1 H), 6.98 (d, J = 7.6 Hz, 1 H), 6.38-6.42 (m, 3 H), 6.07 (s, 2 H), 4.51 (d, J = 13.2 Hz, 2 H), 2.84-2.91 (m, 4 H), 1.71 (d, J = 12 Hz, 4 H),1.40 (d, J = 6.8 Hz, 2 H), 1.05 (d, J = 11.6 Hz, 2 H), HPLC purity 98.54%. LC-MS (ES) m/z = 403.2 [M+H]⁺.

Example 11: N-(2-(1-(2-(pyridin-2-ylamino)pyrimidin-4-yl)piperidin-4-yl)ethyl)sulfamide

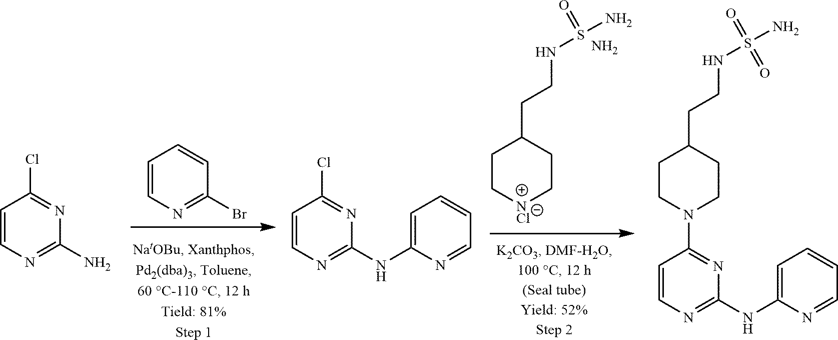

### Step 1: Synthesis of 4-Chloro-N-(Pyridin-2-yl)Pyrimidin-2-Amine

Procedure: To a stirred solution of Xanthphos (0.17 g, 0.30 mmol) inToluene (25 mL) was added Pd₂(dba)₃ (0.14 g, 0.15 mmol) and the reaction mixture was purged with nitrogen gas for 30 minutes. Then heated the reaction mixture at 60° C. Once temperature was attained, added sodium tertiary butoxide (1.1 g, 11.5 mmol) and 2-bromopyridine (1.1 mL, 11.5 mmol) at 60° C. under inert atmosphere. Finally added 2-amino-4-chloropyrimidine (1 g, 7.70 mmol) and resulting reaction mixture was heated at 110° C. for 12 h. After the completion of the reaction, monitored by TLC using 20% ethyl acetate in hexane as eluent, reaction was diluted with ethyl acetate (50 mL) and filtered through diatomaceous earth, filtrate was washed with water (2*30 mL) and brine (30 mL) and separated the organic layer. The combined organic layer was dried over anhydrous sodium sulphate, filtered and evaporated under reduced pressure. The crude obtained was purified by combiflash purifier using 20% ethyl acetate in hexane as eluent to afford 4-chloro-N-(pyridin-2-yl)pyrimidin-2-amine(1.3 g, 81 %). ¹HNMR (400 MHz, DMSO-d₆): δ 10.21 (s, 1 H), 8.48 (d, J= 4.8 Hz, 1 H), 8.26-8.32 (m, 1 H), 8.11 (d, J= 8.4 Hz, 1 H), 7.76 (t, J= 8 Hz, 1 H), 7.0-7.08 (m, 2 H). LC-MS (ES) m/z = 207.1 [M+H]⁺.

### Step 2: Synthesis of N-(2-(1-(2-(Pyridin-2-Ylamino)Pyrimidin-4-yl)Piperidin-4-yl)Ethyl)Sulfamide

Procedure: To a stirred solution of 4-chloro-N-(pyridin-2-yl)pyrimidin-2-amine(0.1 g, 0.48 mmol) inDMF (5 mL) was added(2-(piperidin-4-yl)ethyl)sulfamide hydrochloride (0.14 g, 0.58 mmol) and aqueous solution of potassium carbonate (0.132 g, 0.96 mmol) and the resulting reaction mixture was stirred at 100° C. for 12 h. The progress of the reaction was monitored by TLC using 5% MeOH-DCM as eluent. After the completion of the reaction, quenched with water (20 mL) and extracted with ethyl acetate (2 × 30 mL). The combined organic layers were washed with brine (30 mL), dried over sodium sulphate, filtered and evaporated under reduced pressure. The crude obtained was purified by combiflash purifier using 5% MeOH-DCM as eluent to afford N-(2-(1-(2-(pyridin-2-ylamino)pyrimidin-4-yl)piperidin-4-yl)ethyl)sulfamide (0.096 g, 52%). ¹HNMR (400 MHz, DMSO-d₆): δ 8.82 (s, 1 H), 8.20 (s, 2 H), 7.95 (d, J= 5.6 Hz, 1 H), 7.68 (t, J= 7.6 Hz, 1 H), 6.88-6.92 (m, 1 H), 6.42 (s, 2 H), 6.36-6.40 (m, 1 H), 6.32-6.36 (m, 1 H), 4.30-4.40 (m, 2 H), 2.80-2.95 (m, 4 H), 1.60-1.75 (m, 3 H), 1.35-1.45 (m, 2 H), 1.0-1.10 (m, 2 H). LC-MS (ES) m/z = 378.2 [M+H]⁺.

Example 12: N-(2-(1-(2-(pyridin-3-ylamino)pyrimidin-4-yl)piperidin-4-yl)ethyl)sulfamide

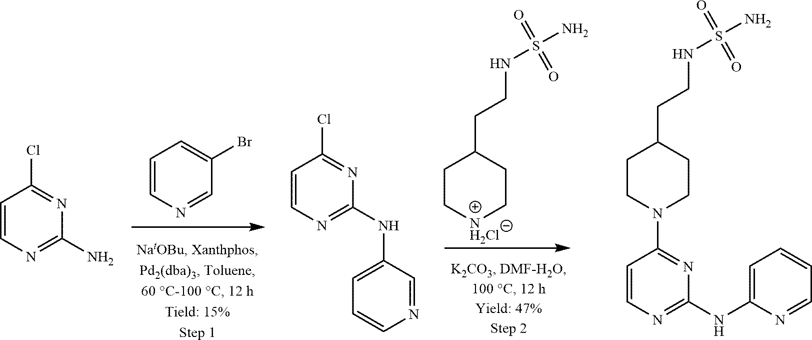

### Step 1: 4-Chloro-N-(Pyridin-3-yl)Pyrimidin-2-Amine

Procedure: To a stirred solution of Xanthphos (0.087 g, 0.15 mmol) in toluene (20 mL) was added Pd₂(dba)₃ (0.069 g, 0.076 mmol) and the reaction mixture was purged with nitrogen gas for 30 minutes. Then heated the reaction mixture at 60° C. Once temperature was attained, added sodium tertiary butoxide (0.36 g, 5.70 mmol) and 3-bromopyridine (0.54 mL, 5.70 mmol) at 60° C. under inert atmosphere. Finally added 2-amino-4-chloropyrimidine (0.5 g, 3.80 mmol) and resulting reaction mixture was heated at 110° C. for 12 h. After the completion of the reaction, monitored by TLC using 30% ethyl acetate in hexane as eluent, reaction was diluted with ethyl acetate (50 mL) and filtered through diatomaceous earth, filtrate was washed with water (2*30 mL) and brine (30 mL) and separated the organic layer. The combined organic layer was dried over anhydrous sodium sulphate, filtered and evaporated under reduced pressure. The crude obtained was purified by combiflash purifier using 30% ethyl acetate in hexane as eluent to afford 4-chloro-N-(pyridin-3-yl)pyrimidin-2-amine (0.12 g, 15%). ¹HNMR (400 MHz, DMSO-d₆): δ 10.17 (s, 1 H), 8.82-8.84 (m, 1 H), 8.45 (d, J= 5.2 Hz, 1H), 8.18 (d, J= 4.4 Hz, 1 H), 8.12 (d, J= 8.4 Hz, 1 H), 7.30-7.36 (m, 1 H), 7.0 (d, J= 5.2 Hz, 1 H). LC-MS (ES) m/z = 207.0 [M+H]⁺.

### Step 2: N-(2-(1-(2-(Pyridin-3-Ylamino)Pyrimidin-4-yl)Piperidin-4-yl)Ethyl)Sulfamide

Procedure: To a stirred solution of 4-chloro-N-(pyridin-3-yl)pyrimidin-2-amine(0.1 g, 0.48 mmol) in DMF (5 mL) was added(2-(piperidin-4-yl)ethyl)sulfamide hydrochloride (0.14 g, 0.58 mmol) and aqueous solution of potassium carbonate (0.132 g, 0.96 mmol) and the resulting reaction mixture was stirred at 100° C. for 12 h. The progress of the reaction was monitored by TLC using 5% MeOH-DCM as eluent. After the completion of the reaction, quenched with water (20 mL) and extracted with ethyl acetate (2 x 30 mL). The combined organic layers were washed with brine (30 mL), dried over sodium sulphate, filtered and evaporated under reduced pressure. The crude obtained was purified by combiflash purifier using 5% MeOH-DCM as eluent to afford N-(2-(1-(2-(pyridin-3-ylamino)pyrimidin-4-yl)piperidin-4-yl)ethyl)sulfonicamide as off-white solid (0.086 g, 47%). ¹HNMR (400 MHz, DMSO-d₆): δ 9.13 (s, 1 H), 8.83 (s, 1 H), 8.11 (d, J= 8 Hz, 1 H), 8.04-8.08 (m, 1 H), 7.93 (d, J= 6 Hz, 1 H), 7.20-7.26 (m, 1 H), 6.42 (s, 2 H), 6.36-6.40 (m, 1 H), 6.28 (d, J = 6 Hz, 1 H), 4.25-4.35 (m, 2 H), 2.80-2.95 (m, 4 H), 1.60-1.75 (m, 3 H), 1.35-1.45 (m, 2 H), 1.0-1.15 (m, 2 H).LC-MS (ES) m/z = 378.2 [M+H]⁺.

Example 13: N-(2-(1-(2-(pyridin-4-ylamino)pyrimidin-4-yl)piperidin-4-yl)ethyl)sulfamide

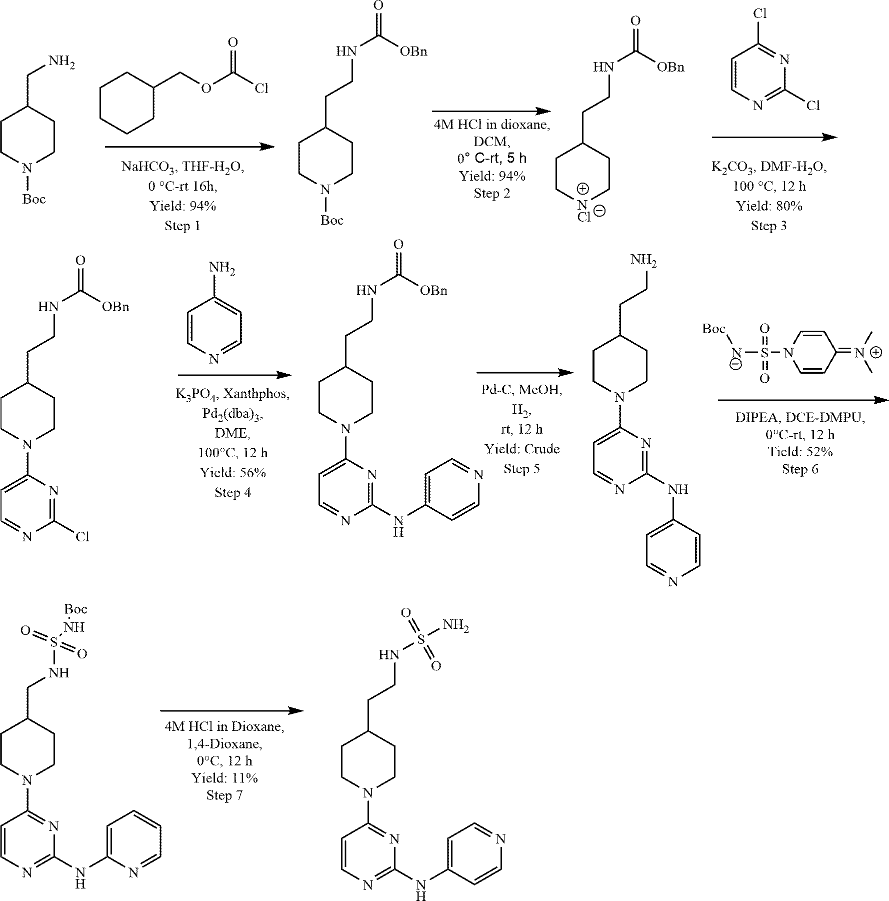

### Step 1: Tert-Butyl 4-(2-(((Benzyloxy)Carbonyl)Amino)Ethyl)Piperidine-1-Carboxylate

Procedure: To a stirred solution of tert-butyl 4-(2-aminoethyl)piperidine-1-carboxylate (4 g, 17.5 mmol) in THF (80 mL) was added sodium bicarbonate (4.4 g, 52.5 mmol) in water (10 mL) at 0° C. followed by added benzyl chloroformate (3.7 mL, 26.2 mmol) and the resulting reaction mixture was stirred for 12 h at room temperature. The progress of the reaction was monitored by TLC using 30% EtOAc in n-hexane as eluent. Then the reaction was quenched with water (30 mL) and extracted with ethyl acetate (2 x 50 mL). The combined organic layers were washed with brine (50 mL), dried over sodium sulphate, filtered and evaporated under reduced pressure. The crude was purified by combiflash purifier using 30% ethyl acetate in n-hexane as eluent to afford tert-butyl 4-(2-(((benzyloxy)carbonyl)amino)ethyl)piperidine-1-carboxylate as off-white solid (6 g, 94%). ¹HNMR (400 MHz, DMSO-d₆): δ 7.29-7.40 (m, 5 H), 7.16-7.40 (m, 1 H), 5.11 (s, 2 H), 3.80-3.95 (m, 2 H), 2.95-3.05 (m, 2 H), 2.60-2.75 (m, 2 H), 1.55-1.65 (m, 2 H), 1.37 (s, 9 H), 1.30-1.36 (m, 3 H), 0.85-0.95 (m, 2 H). LC-MS (ES) m/z = 263.2 [M+H-100]⁺.

### Step 2: Benzyl (2-(Piperidin-4-yl)Ethyl)Carbamate Hydrochloride

Procedure: To a stirred solution of tert-butyl 4-(2-(((benzyloxy)carbonyl)amino)ethyl)piperidine-1-carboxylate(6 g, 16.5 mmol) in DCM (30 mL) was added 4M HCl in dioxane (15 mL) at 0° C. and the resulting reaction mixture was stirred for 5 h at room temperature. The progress of the reaction was monitored by TLC. After completion of the reaction, the reaction mixture was evaporated under reduced pressure. The residue obtained was triturated with toluene (2*50 mL) to afford benzyl (2-(piperidin-4-yl)ethyl)carbamate hydrochloride as off-white solid (4.53 g, 92%). ¹HNMR (400 MHz, DMSO-d₆): δ 8.44 (bs, 2 H), 7.29-7.37 (m, 5 H), 7.18-7.22 (m, 1 H), 4.99 (s, 2 H), 3.20 (d, J= 12.4 Hz, 2H), 3.01 (q, J= 6.8 Hz, 2 H), 2.76 (t, J= 12.8 Hz, 2 H), 1.77 (d,J= 13.2 Hz, 2 H), 1.45-1.55 (m, 1 H), 1.34 (q, J = 6.8 Hz, 2 H), 1.15-1.30 (m, 2 H). LC-MS (ES) m/z = 263.2 [M+H]⁺.

### Step 3: Benzyl (2-(1-(2-Chloropyrimidin-4-yl)Piperidin-4-yl)Ethyl)Carbamate

Procedure: To a stirred solution of benzyl (2-(piperidin-4-yl)ethyl)carbamate hydrochloride (1 g, 3.30 mmol) in DMF was added 2,4-dichloropyrimidine (0.74 g, 5.0 mmol) followed by added aqueous solution of potassium carbonate (1.36 g, 9.90 mmol) in water (1 mL) and the resulting reaction mixture was heated at 100° C. for 12 h. After completion of the reaction monitored by TLC using 30% ethyl acetate in n-hexane as eluent, to the reaction mixture was added water (30 mL) and extracted with ethyl acetate (2*50 mL) and separated the organic layer. The combined organic layer was washed with brine (30 mL), dried over sodium sulphate, filtered and evaporated under reduced pressure. The obtained crude was purified by combiflash purifier using 30% ethyl acetate in n-hexane as eluent to afford benzyl (2-(1-(2-chloropyrimidin-4-yl)piperidin-4-yl)ethyl)carbamate (1 g, 80%). ¹HNMR (400 MHz, DMSO-d₆): δ 7.99 (d, J= 6 Hz, 1 H), 7.29-7.37 (m, 5 H), 7.16-7.22 (m, 1 H), 6.79 (d, J= 6 Hz, 1 H), 4.99 (s, 2 H), 4.25-4.35 (m, 2 H), 2.97-3.12 (m, 2 H), 2.80-2.90 (m, 2 H), 1.65-1.75 (m, 2 H), 1.55-1.65 (m, 1 H), 1.30-1.40 (m, 2 H), 0.99-1.07 (m, 2H). LC-MS (ES) m/z = 375.2 [M+H]⁺.

### Step 4: Benzyl (2-(1-(2-(Pyridin-4-Ylamino)Pyrimidin-4-yl)Piperidin-4-yl)Ethyl)Carbamate

Procedure: To a stirred solution of benzyl (2-(1-(2-chloropyrimidin-4-yl)piperidin-4-yl)ethyl)carbamate (0.8 g, 2.10 mmol) in DME (20 mL) was added 4-aminopyridine (0.4 g, 4.20 mmol) and potassium phosphate tribasic (1.33 g, 6.30 mmol) and xanthphos (0.12 g, 0.21 mmol) and the resulting reaction mixture was purged with nitrogen gas for 30 minutes followed by adding Pd₂(dba)₃ (0.096 g, 0.10 mmol) under inert atmosphere and the reaction mixture was heated at 100° C. for 12 h.. After the completion of the reaction, monitored by TLC using 5% methanol in dichloromethane as eluent, the reaction mixture was filtered through diatomaceous earth; filtrate was diluted with water (30 mL) and extracted with ethyl acetate (2*50 mL) and separated the organic layer. The combined organic layer was washed with brine (30 mL), dried over anhydrous sodium sulphate, filtered and evaporated under reduced pressure. The obtained crude was purified by combiflash purifier using 5% methanol in dichloromethane as eluent to afford benzyl (2-(1-(2-(pyridin-4-ylamino)pyrimidin-4-yl)piperidin-4-yl)ethyl)carbamate as off white solid (0.52 g, 56%). ¹HNMR (400 MHz, DMSO-d₆): δ 9.42 (s, 1 H), 8.26 (d, J= 6.4 Hz, 1 H), 7.98 (d, J= 6 Hz, 1 H), 7.67 (d, J= 6.4 Hz, 1 H), 7.27-7.36 (m, 5 H), 7.18-7.24 (m, 1 H), 6.35 (d, J= 6.4 Hz, 1 H), 4.99 (s, 2 H), 4.30-4.40 (m, 2 H), 3.04 (q, J= 6.8 Hz, 2 H), 2.85 (t, J= 12 Hz, 2 H), 1.73 (d, J= 12.8 Hz, 2 H), 1.55-1.65 (m, 1 H), 1.35 (q, J = 6.8 Hz, 2 H), 1.01-1.09 (m, 2 H). LC-MS (ES) m/z = 433.4 [M+H]⁺.

### Step 5: 4-(4-(2-Aminoethyl)Piperidin-1-yl)-N-(Pyridin-4-yl)Pyrimidin-2-Amine

Procedure: To a stirred solution of benzyl (2-(1-(2-(pyridin-4-ylamino)pyrimidin-4-yl)piperidin-4-yl)ethyl)carbamate (0.2 g, 0.46 mmol) in methanol (10 mL) was added 10% palladium on carbon (0.05 g) and the resulting reaction mixture was stirred for 12 h at room temperature. After the completion of the reaction, monitored by TLC using 5% methanol in dichloromethane as eluent, the reaction mixture was filtered through diatomaceous earth, filtrate was evaporated under reduced pressure to afford 4-(4-(2-aminoethyl)piperidin-1-yl)-N-(pyridin-4-yl)pyrimidin-2-amine as pale yellow semi solid (0.12 g, crude) ¹HNMR (400 MHz, DMSO-d₆): δ 9.42 (s, 1 H), 8.26 (d, J= 6.4 Hz, 1 H), 7.98 (d, J= 6 Hz, 1 H), 7.67 (d, J= 6.4 Hz, 1 H), 7.27-7.36 (m, 5 H), 7.18-7.24 (m, 1 H), 6.35 (d, J= 6.4 Hz, 1 H), 4.99 (s, 2 H), 4.30-4.40 (m, 2 H), 3.04 (q, J= 6.8 Hz, 2 H), 2.85 (t, J= 12 Hz, 2 H), 1.73 (d, J= 12.8 Hz, 2 H), 1.55-1.65 (m, 1 H), 1.35 (q, J= 6.8 Hz, 2 H), 1.01-1.09 (m, 2 H). LC-MS (ES) m/z = 299.0 [M+H]⁺.

### Step 6: Synthesis of Tert-Butyl (N-(2-(1-(2-(Pyridin-4-Ylamino)Pyrimidin-4-yl)Piperidin-4-yl)Ethyl)Sulfamoyl)Carbamate

Procedure: To a stirred solution of4-(4-(2-aminoethyl)piperidin-1-yl)-N-(pyridin-4-yl)pyrimidin-2-amine (0.12 g, 0.40 mmol) in 1,2-dichloroethane (6 mL) and 1,3-Dimethyl-3,4,5,6-tetrahydro-2-pyrimidinone (1 mL) was added N,N-diisopropylethylamine (0.2 mL, 1.20 mmol) at 0° C. and the resulting reaction mixture was stirred for 10 minutes at 0° C. followed by added (tert-butoxycarbonyl)((4-(dimethyliminio)pyridin-l(4H)-yl)sulfonyl)amide (0.13 g, 0.44 mmol) and the reaction mixture was stirred for 12 h at room temperature. After completion of the reaction monitored by TLC using 5% methanol in dichloroethane as eluent, to the reaction mixture was added water (20 mL) and extracted with dichloroethane (2*50 mL) and separated the organic layer. The combined organic layer was washed with brine (30 mL), dried over sodium sulphate, filtered and evaporated under reduced pressure. The obtained crude was purified by combiflash purifier using 5% methanol in dichloroethane as eluent to afford tert-butyl (N-(2-(1-(2-(pyridin-4-ylamino)pyrimidin-4-yl)piperidin-4-yl)ethyl)sulfamoyl)carbamate (0.11 g, 52%). ¹HNMR (400 MHz, DMSO-d₆): δ 10.79 (s, 1 H), 9.45 (s, 1 H), 8.26 (d, J= 5.6 Hz, 1 H), 7.98 (d, J= 6 Hz, 1 H), 7.68 (d, J= 6 Hz, 2 H), 7.42-7.48 (m, 1 H), 6.36 (d, J = 6 Hz, 1 H), 4.30-4.45 (m, 2 H), 2.78-2.93 (m, 4 H), 1.65-1.75 (m, 3 H), 1.41 (s, 9 H), 1.35-1.40 (m, 2 H), 1.0-1.1 (m, 2 H). LC-MS (ES) m/z = 478.2 [M+H]⁺.

### Step 7: N-(2-(1-(2-(Pyridin-4-ylamino)Pyrimidin-4-yl)Piperidin-4-yl)Ethyl)Sulfamide

Procedure: To a stirred solution of tert-butyl (N-(2-(1-(2-(pyridin-4-ylamino)pyrimidin-4-yl)piperidin-4-yl)ethyl)sulfamoyl)carbamate in 1,4-dioxane (6 mL) was added 4M HCl in dioxane (4 mL) at 0° C. and the resulting reaction mixture was stirred for 12 h at room temperature. After completion of the reaction, monitored by TLC using 10% methanol in dichloroethane as eluent, reaction mixture was evaporated under reduced pressure. The residue obtained was dissolved in water (5 mL) and aqueous layer was washed with ethyl acetate (2* 10 mL). Further aqueous layer was basified with saturated aqueous solution of sodium bicarbonate to pH ~ 8 to 9 and extracted with 5% MeOH-DCM (3*50 mL) and separated the organic layer. The combined organic layer was dried over anhydrous sodium sulphate, filtered and evaporated under reduced pressure. The obtained crude was purified by Prep-HPLC using 5% methanol in dichloroethane as eluent to afford N-(2-(1-(2-(pyridin-4-ylamino)pyrimidin-4-yl)piperidin-4-yl)ethyl)sulfamide as off white solid (0.009 g, 11%). ¹HNMR (400 MHz, DMSO-d₆): δ 9.50 (s, 1 H), 8.27 (d, J= 5.2 Hz, 2 H), 7.98 (d, J= 6 Hz, 1 H), 7.70 (d, J= 6.8 Hz, 2 H), 6.43 (s, 2 H), 6.38 (t, J= 7.2 Hz, 2 H), 4.30-4.40 (m, 2 H), 2.85-2.95 (m, 4 H), 1.60-1.75 (m, 3 H), 1.35-1.45 (m, 2 H), 1.0-1.15 (m, 2 H). LC-MS (ES) m/z = 378.2 [M+H]⁺.

Example 14: Synthesis of N-(2-(1-(2-(pyridin-3-yl)pyrimidin-4-yl)piperidin-4-yl)ethyl)sulfamide

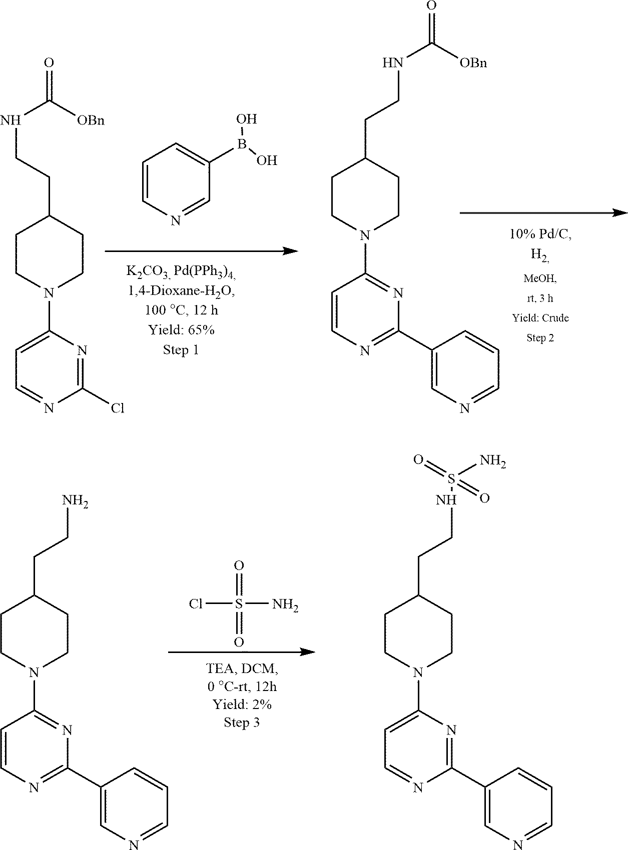

### Step 1: Synthesis of Benzyl (2-(1-(2-(Pyridin-3-yl)Pyrimidin-4-yl)Piperidin-4-yl)Ethyl) Carbamate

Procedure: To a stirred solution of benzyl (2-(1-(2-chloropyrimidin-4-yl)piperidin-4-yl)ethyl)carbamate (0.36 g, 0.96 mmol) in1,4-dioxane (10 mL) was addedpyridin-3-ylboronic acid (0.17 g, 1.40 mmol) and potassium carbonate (0.39 g, 2.80 mmol) and the resulting reaction mixture was purged with nitrogen gas for 30 minutes followed by addition of Pd(PPh₃)₄ (0.11 g, 0.096 mmol) under inert atmosphere and the reaction mixture was heated at 100° C. for 12 h. After the completion of the reaction, the reaction mixture was filtered through diatomaceous earth; filtrate was diluted with water (30 mL) and extracted with ethyl acetate (2x50 mL). The combined organic layer was washed with brine (30 mL), dried over anhydrous sodium sulphate, filtered and evaporated under reduced pressure. The obtained crude was purified by combiflash purifier using 5% methanol in dichloromethane as eluent to afford benzyl (2-(1-(2-(pyridin-3-yl)pyrimidin-4-yl)piperidin-4-yl)ethyl)carbamate as an off white solid (0.26 g, 65 %). ¹HNMR (400 MHz, DMSO-d₆): δ 9.42 (s, 1 H), 8.62-8.68 (m, 1 H), 8.57 (d, J= 8 Hz, 1 H), 8.27 (d, J= 6 Hz, 1 H), 7.46-7.52 (m, 1 H), 7.28-7.38 (m, 5 H), 7.18-7.24 (m, 1 H), 6.79 (d, J= 6.4 Hz, 1 H), 4.99 (s, 2 H), 4.45-4.55 (m, 2 H), 3.05-3.10 (m, 2 H), 2.90 (t, J= 12.4 Hz, 2 H), 1.75-1.85 (m, 2 H), 1.55-1.65 (m, 1 H), 1.36 (q, J= 6.8 Hz, 2 H), 1.05-1.15 (m, 2 H). LC-MS (ES) m/z = 418.2 [M+H]⁺. Step 2: Synthesis of 2-(1-(2-(pyridin-3-yl)pyrimidin-4-yl)piperidin-4-yl)ethan-1-amine

Procedure: To a stirred solution of benzyl (2-(1-(2-(pyridin-3-yl)pyrimidin-4-yl)piperidin-4-yl)ethyl)carbamate (0.26 g, 0.62 mmol) in methanol (10 mL) was added 10% palladium on carbon (0.03 g) and the resulting reaction mixture was stirred for 12 h at room temperature under hydrogen atmosphere. After the completion of the reaction, monitored by TLC using 5% methanol in dichloromethane as eluent, the reaction mixture was filtered through diatomaceous earth, filtrate was evaporated under reduced pressure to afford 2-(1-(2-(pyridin-3-yl)pyrimidin-4-yl)piperidin-4-yl)ethan-1-amine as colorless semi-solid (0.17 g, crude). LC-MS(ES) m/z = 284.2 [M+H]⁺.

### Step 3: Synthesis of N-(2-(1-(2-(pyridin-3-yl)pyrimidin-4-yl)piperidin-4-yl)ethyl)sulfamide

Procedure: To a stirred solution of 2-(1-(2-(pyridin-3-yl)pyrimidin-4-yl)piperidin-4-yl)ethan-1-amine (0.17 g, 0.59 mmol) in DCM (10 mL) was added triethylamine (0.24 mL, 1.77 mmol) at 0° C. and the resulting reaction mixture was stirred for 10 minutes at 0° C. followed by added sulfamoyl chloride (0.10 g, 0.89 mmol) and the reaction mixture was stirred for 12 h at room temperature. After completion of the reaction, the reaction mixture was quenched with water (20 mL) and extracted with dichloroethane (2 x 50 mL). The combined organic layer was washed with brine (30 mL), dried over sodium sulphate, filtered and evaporated under reduced pressure. The obtained crude was purified by combiflash purifier using 5% methanol in dichloroethane as eluent to afford N-(2-(1-(2-(pyridin-3-yl)pyrimidin-4-yl)piperidin-4-yl)ethyl)sulfamide as an off-white solid (0.005 g, 2%). ¹HNMR (400 MHz, DMSO-d₆): δ 9.42 (s, 1 H), 8.62-8.66 (m, 1 H), 8.57 (d, J= 7.6 Hz, 1 H), 8.27 (d, J= 6.4 Hz, 1 H), 7.46-7.50 (m, 1 H), 6.80 (d, J= 6.4 Hz, 1 H), 6.43 (s, 2 H), 6.36-6.42 (m, 1 H), 4.45-4.55 (m, 2 H), 2.85-2.95 (m, 4 H), 1.65-1.80 (m, 3 H), 1.35-1.45 (m, 2 H), 1.05-1.15 (m, 2 H), 1.0-1.1 (m, 2 H). LC-MS (ES) m/z = 363.3 [M+H]⁺; HPLC purity: 99.21%.

Example 15: N-(2-(1-(5,6-dimethylpyrimidin-4-yl)piperidin-4-yl)ethyl)sulfamide

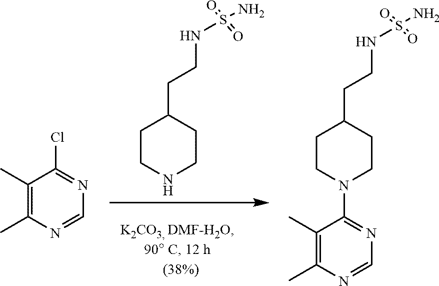

Procedure: To a stirred solution of 4-chloro-5,6-dimethylpyrimidine (0.1 g, 0.70 mmol) in DMF (5 mL) was added(2-(piperidin-4-yl)ethyl)sulfamide hydrochloride (0.2 g, 0.84 mmol) and aqueous solution of potassium carbonate (0.145 g, 1.05 mmol) and the resulting reaction mixture was stirred at 90° C. for 12 h. The progress of the reaction was monitored by TLC. Then the reaction was quenched with water (20 mL) and extracted with ethyl acetate (2 x 30 mL). The combined organic layers were washed with brine (30 mL), dried over sodium sulphate, filtered and evaporated under reduced pressure. The crude obtained was purified by combiflash purifier using methanol in dichloromethane to afford N-(2-(1-(5,6-dimethylpyrimidin-4-yl)piperidin-4-yl)ethyl)sulfamide (0.085 g, 38 %). ¹HNMR (400 MHz, DMSO-d₆): δ 8.36 (s, 1 H), 6.42 (s, 2 H), 6.38 (t, J= 6.4 Hz, 1 H), 3.61-3.65 (m, 2 H), 2.90 (q, J= 6.4 Hz, 2 H), 2.73 (t, J= 12 Hz, 2 H), 2.29 (s, 3 H), 2.07 (s, 3 H), 1.65-1.75 (m, 2 H), 1.45-1.57(m, 1 H), 1.42 (q, J = 6.8 Hz, 2 H), 1.15-1.25 (m, 2 H). LC-MS (ES) m/z = 314.2 [M+H]⁺.

Example 16: N-(2-(1-(6-methylpyrimidin-4-yl)piperidin-4-yl)ethyl)sulfamide

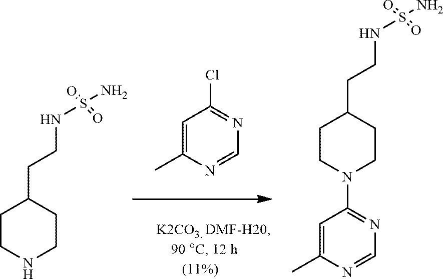

Procedure: To a stirred solution of compound (2-(piperidin-4-yl)ethyl)sulfamide hydrochloride (0.1 g, 0.41 mmol) in DMF (4 mL) was added 4-chloro-6-methylpyrimidine (0.058 g, 0.45 mmol) and aqueous solution of potassium carbonate (0.113 g, 0.82 mmol) and the resulting reaction mixture was stirred at 90° C. for 12 h. The progress of the reaction was monitored by TLC. Then the reaction was quenched with water (20 mL) and extracted with ethyl acetate (2 x 30 mL). The combined organic layers were washed with brine (30 mL), dried over sodium sulphate, filtered and evaporated under reduced pressure. The crude obtained was purified by combiflash purifier using ethyl acetate in n-hexane to afford N-(2-(1-(6-methylpyrimidin-4-yl)piperidin-4-yl)ethyl)sulfamide (0.011 g , 11.4 %). ¹HNMR (400 MHz, DMSO-d₆): δ 8.31 (s, 1H), 6.65 (s, 1 H), 6.42 (s, 2 H), 6.36-6.39 (m, 1 H), 4.30-4.40 (m, 2 H), 2.89 (q, J= 7.2 Hz, 2 H), 2.81 (t, J= 11.2 Hz, 2 H), 2.21(s, 3 H), 1.62-1.70 (m, 3 H), 1.38 (q, J = 7.2 Hz, 2 H), 0.96-1.05 (m, 2 H). LC-MS (ES) m/z = 300.1 [M+H]⁺.

Example 17: N-(2-(1-(5-methylpyrimidin-4-yl)piperidin-4-yl)ethyl)sulfamide

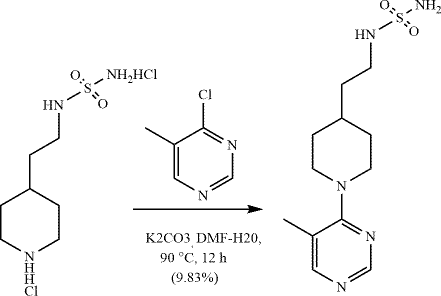

Procedure: To a stirred solution of compound (2-(piperidin-4-yl)ethyl)sulfamide hydrochloride (0.1 g, 0.41 mmol) in DMF (4 mL) was added 4-chloro-5-methylpyrimidine (0.058 g, 0.45 mmol) and aqueous solution of potassium carbonate (0.113 g, 0.82 mmol) and the resulting mixture was stirred at 90° C. for 12 h. The progress of the reaction was monitored by TLC. Then the reaction was quenched with water (20 mL) and extracted with ethyl acetate (2 x 30 mL). The combined organic layers were washed with brine (30 mL), dried over sodium sulphate, filtered and evaporated under reduced pressure. The crude obtained was purified by combiflash purifier using ethyl acetate in n-hexane to afford N-(2-(1-(5-methylpyrimidin-4-yl)piperidin-4-yl)ethyl)sulfamide (0.012 g, 9.83 %). ¹HNMR (400 MHz, DMSO-d₆): δ 8.44 (s, 1 H), 8.09 (s, 1 H), 6.42 (s, 2 H), 6.39 (t, J= 6 Hz, 1 H), 3.93 (d, J= 13.2 Hz, 1 H), 2.90 (q, J= 7.2 Hz, 2 H), 2.80 (t, J= 12.4 Hz, 2 H), 2.15 (s, 3 H), 1.70 (d, J= 12.8 Hz, 2 H), 1.58-1.63 (m, 1 H), 1.41(q, J= 6.8 Hz, 2 H), 1.11-1.21 (m, 2 H). LC-MS (ES) m/z = 300.1 [M+H]⁺.

Example 18: Synthesis of N-(2-(1-(pyrimidin-4-yl)piperidin-4-yl)ethyl)sulfamide

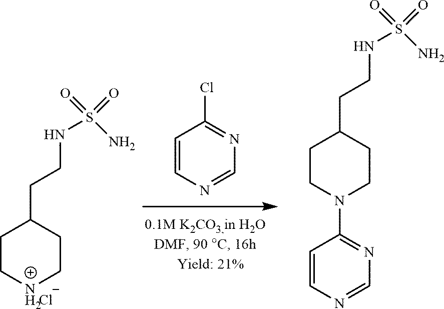

Procedure: To a stirred solution of 4-chloropyrimidine (0.1 g, 0.66 mmol) in DMF (3 mL) were added N-(2-(piperidin-4-yl)ethyl) sulfamide hydrochloride (0.2 g, 0.72 mmol), 0.1 M K₂CO₃ in H₂O (9.9 mL, 0.99 mmol). The reaction mixture was stirred at 90° C. for 16 h and monitored by TLC. The reaction mixture was diluted with ethyl acetate (30mL), washed with water (2 x 10mL) followed by brine (10mL), dried over anhydrous sodium sulphate, filtered and evaporated under reduced pressure. The crude residue was purified by combiflash using 2.5% methanol in dichloromethane to afford N-(2-(1-(pyrimidin-4-yl)piperidin-4-yl)ethyl)sulfamide as white solid (0.04 g, 21%). ¹H NMR (400 MHz, DMSO-d₆): δ 8.43 (s, 1 H), 8.10 (d, J= 6.0 Hz, 1 H), 6.78 (d, J= 6.4 Hz, 1 H), 6.43 (s, 2 H), 6.39 (t, J= 5.6 Hz, 1 H), 6.35 (d, J= 12 Hz, 2 H), 2.92 - 2.81 (m, 4 H), 1.72 - 1.64 (m, 3 H), 1.42 - 1.37 (m, 2 H), 1.07 -0.98 (m, 2 H); LC-MS (ES) m/z = 286.1 [M+H]⁺; HPLC purity: 99.53%.

Example 19: Synthesis of 6,7-dimethoxy-4-(4-(2-(sulfamoylamino)ethyl)piperidin-1-yl)quinoline-3-carboxamide

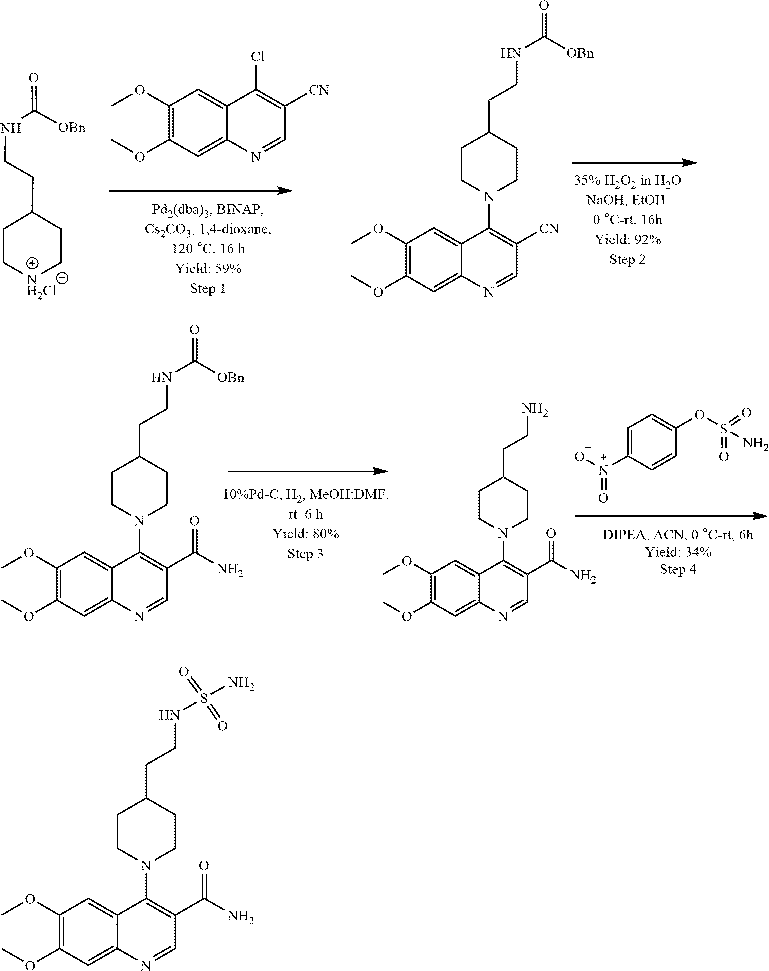

### Step 1: Synthesis of Benzyl (2-(1-(3-Cyano-6,7-Dimethoxyquinolin-4-yl)Piperidin-4-yl)Ethyl)Carbamate

Procedure: To a stirred solution of 4-chloro-6,7-dimethoxyquinoline-3-carbonitrile (0.8 g, 3.22 mmol) in 1,4-dioxane (30 mL) were added benzyl (2-(piperidin-4-yl)ethyl)carbamate hydrochloride (1.0 g, 3.54 mmol), cesium carbonate (3.15 g, 9.67 mmol) and BINAP (0.4 g, 0.64 mmol), resulting mixture was degassed for 15 mins with argon and added Pd₂(dba)₃ (0.29 g, 0.32 mmol) degassed for another 10 mins. The reaction mixture was stirred for 16 h at 120° C. The progress of the reaction was monitored by TLC. Reaction mixture was filtered through celite bed and the filtrate was diluted with ethylacetate (30 mL), washed with water (2 x 10 mL) followed by brine solution (10 mL), dried over anhydrous sodium sulphate, filtered and evaporated under reduced pressure. The crude residue was purified by combiflash using 20-30% ethylacetate in hexane to afford benzyl (2-(1-(3-cyano-6,7-dimethoxyquinolin-4-yl)piperidin-4-yl)ethyl)carb amate as pale yellow solid (0.9 g, 59%). LC-MS (ES) m/z = 475.23 [M+H]⁺.

### Step 2: Synthesis of Benzyl (2-(1-(3-Carbamoyl-6,7-Dimethoxyquinolin-4-yl)Piperidin-4-yl)Ethyl)Carbamate

Procedure: To a stirred solution of benzyl (2-(1-(3-cyano-6,7-dimethoxyquinolin-4-yl)piperidin-4-yl)ethyl)carbamate (0.37 g, 0.78 mmol) in EtOH (10 mL) were added sodium hydroxide (0.062 g, 1.56 mmol) and 35% H₂O₂ in H₂O (5 mL) at 0° C. The resulting mixture was stirred for 16 h at room temperature. The progress of the reaction was monitored by TLC. Reaction mixture was evaporated and resulting residue was diluted with ethyl acetate (30 mL). The organic layer washed with water (2 x 10 mL) followed by brine (10 mL), dried over anhydrous sodium sulphate, filtered and evaporated under reduced pressure to afford benzyl (2-(1-(3-carbamoyl-6,7-dimethoxyquinolin-4-yl)piperidin-4-yl)ethyl)carbamate as colourless liquid (0.35 g, 92%). LC-MS (ES) m/z = 493.2 [M+H]⁺.

### Step 3: Synthesis of 4-(4-(2-Aminoethyl)Piperidin-1-yl)-6,7-Dimethoxyquinoline-3-Carboxamide

Procedure: To a stirred solution of benzyl (2-(1-(3-carbamoyl-6,7-dimethoxyquinolin-4-yl)piperidin-4-yl)ethyl)carbamate (0.35 g, 0.71 mmol) in methanol:DMF (9:1 mL) was added 10% palladium on carbon (0.05 g) and the resulting reaction mixture was stirred for 6 h at room temperature under hydrogen atmosphere. The progress of the reaction was monitored by TLC. The reaction mixture was filtered through diatomaceous earth; organic layer was concentrated under reduced pressure to get 4-(4-(2-aminoethyl)piperidin-1-yl)-6,7-dimethoxyquinoline-3-carboxamide as colourless liquid (0.2 g, 80%). LC-MS (ES) m/z =359.2 [M+H]⁺.

### Step 4: Synthesis of 4-(4-(2-(Hydrosulfonylamino)Ethyl)Piperidin-1-yl)-6,7-Dimethoxy Quinoline-3-Carboxamide

Procedure: To a stirred solution of 4-(4-(2-aminoethyl) piperidin-1-yl)-6,7-dimethoxyquinoline-3-carboxamide (0.16 g, 0.44 mmol) in acetonitrile (10 mL) were added 4-nitrophenyl sulfamate (0.11 g, 0.53 mmol), N,N-Diisopropylethylamine (0.23 mL, 1.34 mmol) at 0° C. The reaction mixture was stirred at room temperature for 6 h, reaction was monitored by TLC. The reaction mixture was evaporated under reduced pressure. The crude residue was purified by preparative HPLC to afford 4-(4-(2-(hydrosulfonylamino)ethyl) piperidin-1-yl)-6,7-dimethoxy quinoline-3-carboxamide as white solid (0.065 g, 34%). ¹H NMR (400 MHz, DMSO-d₆): δ 8.39 (s, 1 H), 7.92 (s, 1 H), 7.50 (s, 1 H), 7.28 (d, J = 7.6 Hz, 2 H), 6.47 - 6. 42 (m, 3 H), 3.90 (s, 6 H), 3.33 - 3.27 (m, 1 H), 3.15 - 3.09 (m, 3 H), 2.95 (d, J = 6.0 Hz, 2 H), 1.78 (d, J = 8.4 Hz, 2 H), 1.63 - 1.50 (m, 3 H), 1.42 (t, J = 9.2 Hz, 2 H); LC-MS (ES) m/z = 438.2 [M+H]⁺; HPLC purity: 98.99%.

Example 20: Synthesis of N-(2-(1-(3-cyano-6,7-dimethoxyquinolin-4-yl)piperidin-4-yl)ethyl)sulfamide

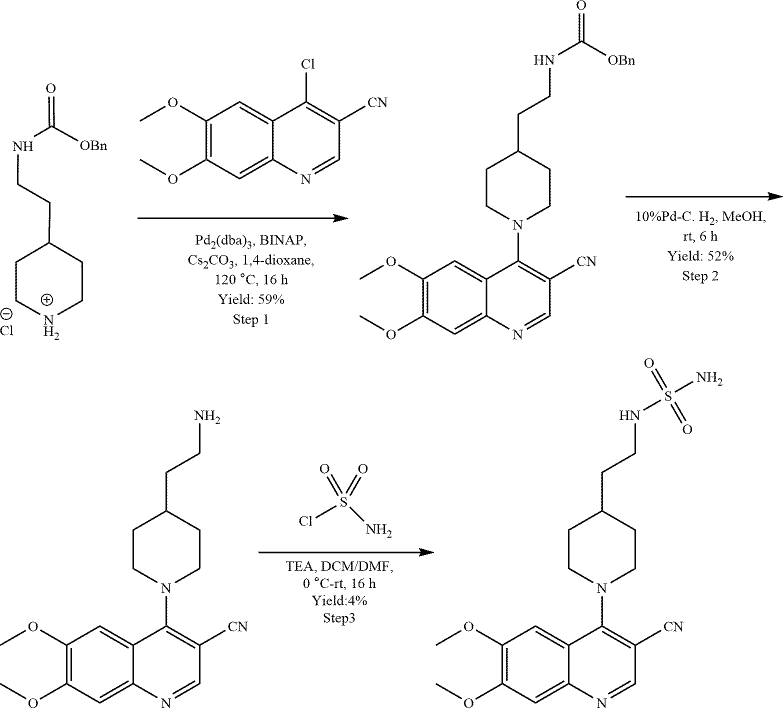

### Step 1: Synthesis of Benzyl (2-(1-(3-cyano-6,7-Dimethoxyquinolin-4-yl)Piperidin-4-yl)Ethyl)Carbamate

Procedure: To a stirred solution of 4-chloro-6,7-dimethoxyquinoline-3-carbonitrile (0.8 g, 3.22 mmol) in 1,4-dioxane (30 mL) were added benzyl (2-(piperidin-4-yl)ethyl)carbamate hydrochloride (1.0 g, 3.54 mmol), cesium carbonate (3.15 g, 9.67 mmol) and BINAP (0.4 g, 0.64 mmol), resulting mixture was degassed for 15 mins with argon and added Pd₂(dba)₃ (0.29 g, 0.32 mmol) degassed for another 10 mins. Resulting mixture was stirred for 16 h at 120° C. Progress of the reaction was monitored by TLC. Reaction mixture was filtered through a celite bed and the filtrate was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 20-30% ethyl acetate in hexane to afford benzyl (2-(1-(3-cyano-6,7-dimethoxyquinolin-4-yl)piperidin-4-yl)ethyl)carbamate as a pale yellow solid (0.9 g, 59%). LC-MS (ES) m/z =475.23 [M+H]⁺.

### Step 2: Synthesis of 4-(4-(2-Aminoethyl)Piperidin-1-yl)-6,7-Dimethoxyquinoline-3-Carbonitrile

Procedure: To a stirred solution of benzyl (2-(1-(3-cyano-6,7-dimethoxyquinolin-4-yl)piperidin-4-yl)ethyl)carbamate (0.5 g, 4.21 mmol) in methanol (10 mL) was added 10% palladium on carbon (0.25 g) and the resulting reaction mixture was stirred for 6 h at room temperature under hydrogen atmosphere. Progress of the reaction was monitored by TLC. The reaction mixture was filtered through diatomaceous earth; organic layer was concentrated under reduced pressure to get 4-(4-(2-aminoethyl)piperidin-1-yl)-6,7-dimethoxyquinoline-3-carbonitrile as colorless liquid (0.185 g, 52%). LC-MS (ES) m/z =341.19 [M+H]⁺.

### Step 3: Synthesis of N-(2-(1-(3-Cyano-6,7-Dimethoxyquinolin-4-yl)Piperidin-4-yl)Ethyl) Sulfamide

Procedure: To a stirred solution of 4-(4-(2-aminoethyl)piperidin-1-yl)-6,7-dimethoxyquinoline-3-carbonitrile (0.2 g, 0.58 mmol) in dichloromethane/DMF (10 mL:5 mL) were added sulfomyl chloride (0.13 g, 1.76 mmol), triethylamine (0.24 mL, 1.76 mmol) at 0° C. Reaction mixture was stirred at room temperature for 16 h, reaction was monitored by TLC. The reaction mixture was diluted with dichloromethane (20 mL), washed with water (2 x 10 mL) followed by brine (10 mL), dried over anhydrous sodium sulphate, filtered and evaporated under reduced pressure. The crude residue was purified by combiflash using 80% ethyl acetate in hexane to afford N-(2-(1-(3-cyano-6,7-dimethoxyquinolin-4-yl)piperidin-4-yl)ethyl)sulfamide as off-white solid (0.01 g, 4%). ¹HNMR (400 MHz, DMSO-d₆): δ 8.57 (s, 1 H), 7.35 (s, 1 H), 7.20 (s, 1 H),6.46 (m, 3 H), 3.93 (s, 3 H), 3.92 (s, 3 H), 3.73 (d, J= 12.4 Hz, 2 H), 3.39 - 3.33 (m, 2 H), 2.97 - 2.94 (m, 2 H), 1.86 (d, J= 11.2 Hz, 2 H), 1.75 - 1.65 (m, 1 H), 1.52 (q, J= 7.2 Hz, 2 H), 1.47 - 1.42 (m, 2 H); LC-MS (ES) m/z =420.16 [M+H]⁺; HPLC purity: 99.8%.

Example 21: Synthesis of N-{2-[1-(6,7-dimethoxyisoquinolin-1-yl)piperidin-4-yl] ethyl}aminosulfonamide

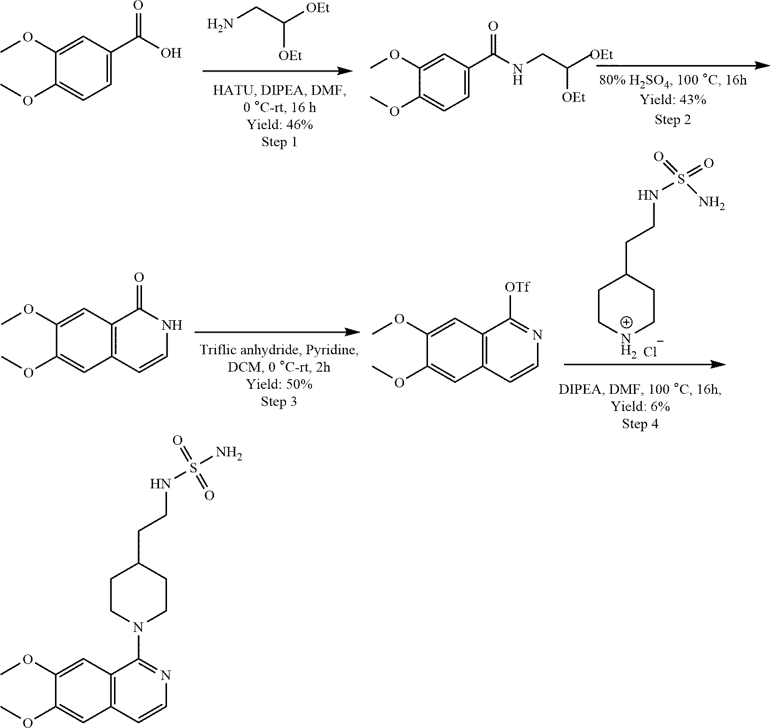

### Step 1: Synthesis of N-(2,2-Diethoxyethyl)-3,4-Dimethoxybenzamide

Procedure: To a stirred solution of 3,4-dimethoxybenzoic acid (2 g, 10.98 mmol) in THF (20 mL) were added 2,2-diethoxyethan-1-amine (1.6 g, 12.08 mmol), HATU (8.35 g, 21.97 mmol) and N,N-Diisopropylethylamine (5.67 mL, 32.96 mmol) at 0° C. and the resulting reaction mixture was stirred for 16 h at room temperature. The progress of the reaction was monitored by TLC using 50% EtOAc in n-hexane as eluent. Then the reaction was quenched with ice-cold water (30 mL) and extracted with ethyl acetate (2 x 30 mL). The combined organic layers were washed with brine (20 mL), dried over sodium sulphate, filtered and evaporated under reduced pressure. The crude residue was purified by combiflash using 30% ethyl acetate in hexane to afford N-(2,2-diethoxyethyl)-3,4-dimethoxybenzamide (1.5 g, 46%). ¹H NMR (400 MHz, DMSO-d₆): δ 8.41 - 8.35 (m, 1 H), 7.46 (d, J= 8.4 Hz, 1 H), 7.44 (s, 1 H), 6.99 (d, J= 8.4 Hz, 1 H), 4.60 (t, J= 5.2 Hz, 1 H), 3.79 (s, 6 H), 3.78 - 3.75 (m, 2 H), 3.67 - 3.59 (m, 2 H), 3.51 - 3.43 (m, 2 H), 1.12 (d, J= 9.6 Hz, 6 H).

### Step 2: Synthesis of 6,7-Dimethoxyisoquinolin-1(2H)-One

Procedure: To a stirred solution of N-(2,2-diethoxyethyl)-3,4-dimethoxybenzamide (0.1 g, 0.33 mmol) in 80% H₂SO₄ (1.0 mL) was stirred for 16 h at 100° C. The progress of the reaction was monitored by TLC. After completion of the reaction, reaction mixture was diluted with ice cold water, neutralized with solid sodium carbonate and extracted with ethyl acetate (2 x 20 mL). The combined organic layers were washed with brine (10 mL), dried over sodium sulphate, filtered and evaporated under reduced pressure. The crude residue was purified by combiflash using 3% methanol in dichloromethane to afford 6,7-dimethoxyisoquinolin-1(2H)-one (0.03 g, 43%). LC-MS (ES) m/z = 205.9 [M+H]⁺.

### Step 3: Synthesis of 6,7-Dimethoxyisoquinolin-1-yl Trifluoromethanesulfonate

Procedure: To a stirred solution of 6,7-dimethoxyisoquinolin-1(2H)-one (0.11 g, 0.53 mmol) in dichloromethane (10 mL) was added triflic anhydride (0.13 mL, 0.80 mmol) and pyridine (0.086 mL, 1.07 mmol) at 0° C. Resulting mixture was stirred for 2 h at room temperature. Progress of the reaction was monitored by TLC. Then the reaction was quenched with water (20 mL) and extracted with ethyl acetate (2 x 20 mL). The combined organic layers were washed with brine (10 mL), dried over sodium sulphate, filtered and evaporated under reduced pressure to afford 6,7-dimethoxyisoquinolin-1-yl trifluoromethanesulfonate as pale yellow solid (0.09 g, 50%). LC-MS (ES) m/z = 338.0 [M+H]⁺.

### Step 4: Synthesis of N-(2-(1-(6,7-Dimethoxyisoquinolin-1-yl)Piperidin-4-yl)Ethyl)Sulfamide

Procedure: To a stirred solution of 6,7-dimethoxyisoquinolin-1-yl trifluoromethanesulfonate (0.09 g, 0.26 mmol) in DMF (5 mL) was added 4-(2-(sulfamoylamino)ethyl)piperidin-1-ium chloride (0.081 g, 0.29 mmol), N,N-diisopropylethylamine (0.11 mL, 0.66 mmol) at room temperature. The reaction mixture was stirred for 16 h at 100° C. and monitored by TLC. After the completion of reaction, reaction mixture was diluted with ice-cold water (20 mL) and extracted with ethyl acetate (2 x 20 mL). The combined organic layers were washed with brine (10 mL), dried over sodium sulphate, filtered and evaporated under reduced pressure. The crude residue was purified by preparative HPLC to afford N-(2-(1-(6,7-dimethoxyisoquinolin-1-yl)piperidin-4-yl)ethyl)sulfamide as off-white solid (0.006 g, 6%). ¹HNMR (400 MHz, DMSO-d₆): δ 7.93 (s, 1 H), 7.27 - 7. 24 (m, 3 H), 6.44 - 6.42 (m, 3 H), 3.90 (s, 6 H), 3.65 - 3.62 (m, 2 H), 2.96 - 2.94 (m, 2 H), 2.85 - 2.75 (m, 2 H), 1.83 - 1.80 (m, 2 H), 1.65 - 1.42 (m, 5 H); LC-MS (ES) m/z = 395.2 [M+H]⁺; HPLC purity: 99.73%.

Example 22: N-(2-(1-(7-methyl-7H-purin-6-yl)piperidin-4-yl)ethyl)sulfamide

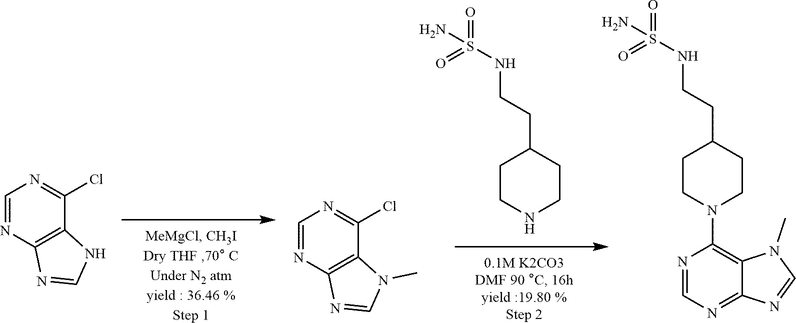

### Step 1: Synthesis of 6-Chloro-7-Methyl-7H-Purine

Procedure: To a stirred solution of 6-chloro-7H-purine (1 g, 6.469 mmol) in Dry THF (20 mL) was added MeMgCl (0.53 g, 7.086 mmol) followed by methyl iodide (1.3 g, 9.158 mmol). Reaction mixture was stirred for 12 h at 70° C. Progress of the reaction was monitored by TLC. Reaction mixture was diluted with water (20 mL), extracted with ethyl acetate (2 x 50 mL). The combined organic layer was washed with brine (10 mL), dried over anhydrous sodium sulphate, filtered and evaporated under reduced pressure. The crude residue was purified by combiflash using methanol in dichloromethane to afford 6-chloro-7-methyl-7H-purine as white solid, (0.4 g, 36.46 %). ¹HNMR (400 MHz, DMSO-d₆): δ 8.75 (s, 1 H),8.71 (s, 1 H), 3.96 (s, 3 H). LC-MS (ES) m/z = 169.1 [M+H]⁺.

### Step 2: Synthesis of N-(2-(1-(9-Methyl-9H-Purin-6-yl)Piperidin-4-yl)Ethyl)Sulfamide

Procedure: To a stirred solution of 6-chloro-7-methyl-7H-purine (0.1 g, 0.598 mmol) in DMF (10 mL) were added N-(2-(piperidin-4-yl)ethyl)sulfamide hydrochloride (0.135 g, 0.65 mmol), 0.1 M K₂CO₃ in H₂O (8.8 mL, 0.88 mmol). Reaction mixture was stirred at 90° C. for 16 h, reaction was monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 2.5% methanol in dichloromethane to afford N-(2-(1-(7-methyl-7H-purin-6-yl)piperidin-4-yl)ethyl)sulfamide as off-white solid (0.04 g, 19.80 %). ¹HNMR (400 MHz, DMSO-d₆): δ 8.42 (s, 1 H), 8.35(s, 1 H), 6.40-6.47 (m, 3 H), 3.95 (s, 3 H), 3.78 (d, J= 12.8 Hz, 2 H), 2.89-2.91(m, 4 H), 1.77(d, J= 11.6 Hz, 2 H), 1.61 (s, 1 H), 1.43-1.48 (m, 2 H), 1.32 (m, 2 H). HPLC purity 100.00%. LC-MS (ES) m/z = 340 [M+H]⁺.

Example 23: Synthesis of N-(2-(1-(9H-purin-6-yl)piperidin-4-yl)ethyl)sulfamide

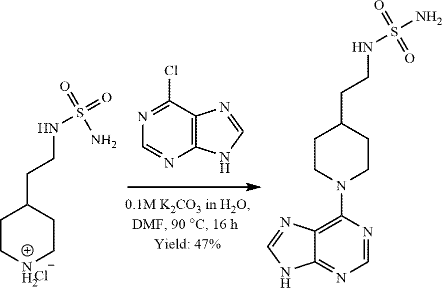

Procedure: To a stirred solution of 6-chloro-9H-purine (0.1 g, 0.649 mmol) in DMF (3 mL) were added N-(2-(piperidin-4-yl)ethyl)sulfamide hydrochloride (0.19 g, 0.714 mmol), 0.1M K₂CO₃ in H₂O (9.74 mL, 0.974 mmol). The reaction mixture was stirred at 90° C. for 16 h and the reaction was monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 70% ethyl acetate in hexane to afford N-(2-(1-(9H-purin-6-yl)piperidin-4-yl)ethyl)sulfamide as off-white solid (0.1 g, 47%). ¹H NMR (400 MHz, DMSO-d₆): δ 12.92 (s, 1 H), 8.15 (s, 1 H), 8.06 (s,1 H), 6.43 (s,2 H), 6.39 (t, J= 6.0 Hz, 1 H), 5.42 - 5.28 (m, 2 H), 3.05 - 2.98 (m, 2 H), 2.90 (q, J= 6.4 Hz, 2 H), 1.78 - 1.62 (m, 3 H), 1.43 - 1.39 (m, 2 H), 1.14 - 1.08 (m, 2 H); LC-MS (ES) m/z = 326.3 [M+H]⁺.

Example 24: N-(2-(1-(9-methyl-9H-purin-6-yl)piperidin-4-yl)ethyl)sulfamide

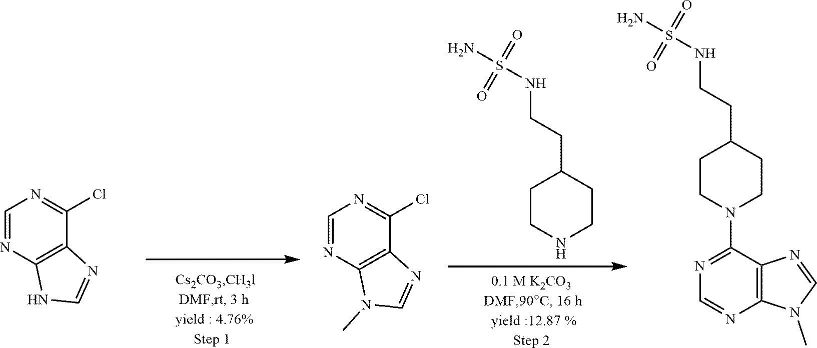

### Step 1: Synthesis of 6-Chloro-9-Methyl-9H-Purine

Procedure: To a stirred solution of 6-chloro-9H-purine (1 g, 6.469 mmol) in DMF (10 mL) was added Cs₂CO₃ (2.31 g, 7.107 mmol) followed by methyl iodide (0.80 mL, 12.93 mmol). Reaction mixture was stirred for 3 h at room temperature. Progress of the reaction was monitored by TLC. Reaction mixture was diluted with water (20 mL), extracted with ethyl acetate (2 x 50mL). The combined organic layer was washed with brine (10mL), dried over anhydrous sodium sulphate, filtered and evaporated under reduced pressure. The crude residue was purified by combiflash using methanol in dichloromethane to afford 6-chloro-9-methyl-9H-purineas off-white solid, (0.1 g, 4.76 %). ¹HNMR (400 MHz, DMSO-d₆): δ 8.76 (s, 1 H),8.62 (s, 1 H), 3.84 (s, 3 H).LC-MS (ES) m/z = 169.1 [M+H]⁺.

### Step 2: Synthesis of N-(2-(1-(9-Methyl-9H-Purin-6-yl)Piperidin-4-yl)Ethyl)Sulfamide

Procedure: To a stirred solution of 6-chloro-9-methyl-9H-purine (0.1 g, 0.595 mmol) in DMF (10 mL) were added N-(2-(piperidin-4-yl)ethyl)sulfamide hydrochloride(0.135 g, 0.65 mmol), 0.1M K₂CO₃ in H₂O (8.8 mL, 0.88 mmol). Reaction mixture was stirred at 90° C. for 16 h, reaction was monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 2.5% methanol in dichloromethane to afford N-(2-(1-(9-methyl-9H-purin-6-yl)piperidin-4-yl)ethyl)sulfamide as off-white solid (0.026 g, 12.87 %).¹HNMR (400 MHz, DMSO-d₆): δ 8.19 (s, 1 H), 8.08 (s, 1 H), 6.37-6.42 (m, 3 H), 5.33 (s, 2 H), 3.69 (s, 3 H), 3.03 (d, J= 11.2 Hz, 2 H), 2.90 (q, J₁= 6.8 Hz, J₂= 7.2 Hz, 2 H), 1.74 (d, J = 12 Hz, 3 H), 1.39 (q, J₁= 6.8 Hz, J₂= 6.8 Hz, 2H), 1.10 (t, J= 10.8 Hz, 2 H).LC-MS (ES) m/z = 340.2 [M+H]⁺.

Example 25: Synthesis of N-(2-(1-(1-methyl-1H-pyrazolo[3,4-d]pyrimidin-4-yl)piperidin-4-yl)ethyl)sulfamide

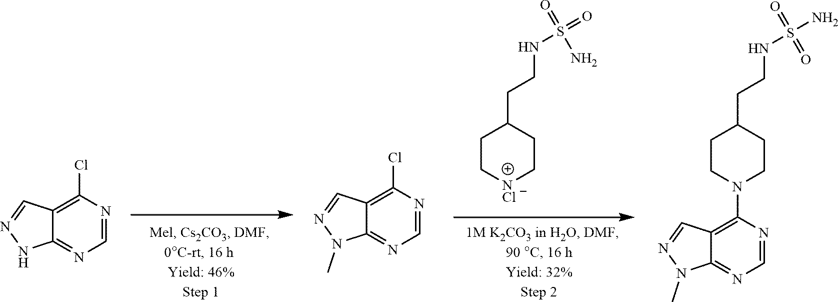

### Step 1 Synthesis of 4-Chloro-1-Methyl-1H-Pyrazolo[3,4-d]Pyrimidine

Procedure: To a stirred solution of 4-chloro-1H-pyrazolo[3,4-d]pyrimidine (1.0 g, 6.49 mmol) in DMF (20 mL) were added Methyl iodide (0.48 mL, 7.79 mmol), cesium carbonate (2.5 g, 7.79 mmol). Resulting mixture was stirred for 16 h at room temperature. The progress of the reaction was monitored by TLC. The reaction mixture was diluted with ice-water (20 mL) and extracted with ethyl acetate (2 x 30 mL). The combined organic layer was washed with brine (10 mL), dried over anhydrous sodium sulphate, filtered and evaporated under reduced pressure. The crude residue was purified by combiflash using 15% ethyl acetate in hexane to afford 4-chloro-1-methyl-1H-pyrazolo[3,4-d]pyrimidine as white solid (0.5 g, 46%). LC-MS (ES) m/z = 169.1 [M+H]⁺.

### Step 2: Synthesis of N-(2-(1-(1-Methyl-1H-Pyrazolo[3,4-d]Pyrimidin-4-yl)Piperidin-4-yl)Ethyl)Sulfamide:

Procedure: To a stirred solution of 4-chloro-1-methyl-1H-pyrazolo[3,4-d]pyrimidine (0.1 g, 0.59 mmol) in DMF (3 mL) were added N-(2-(piperidin-4-yl)ethyl)sulfamide hydrochloride (0.18 g, 0.65 mmol), 0.1M K₂CO₃ in H₂O (8.9 mL, 0.89 mmol). The reaction mixture was stirred at 90° C. for 16 h and the reaction monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 70% ethyl acetate in hexane to afford N-(2-(1-(1-methyl-1H-pyrazolo[3,4-d]pyrimidin-4-yl)piperidin-4-yl)ethyl)sulfamide as white solid (0.065 g, 32%). ¹H NMR (400 MHz, DMSO-d₆): δ 8.24 (s, 1 H), 8.23 (s, 1 H), 6.43 (s, 1 H), 6.40 (t, J= 6.4 Hz, 2 H), 4.75 - 4.62 (m, 2 H), 3.88 (s, 3 H), 3.18 - 3.08 (m, 2 H), 2.91 (q, J = 6.4 Hz, 2 H), 1. 18 - 1.75 (m, 3 H), 1.41 (q, J = 7.2 Hz, 2 H), 1.11 (q, J = 8.4 Hz, 2 H); LC-MS (ES) m/z = 340.2 [M+H]⁺; HPLC purity: 99.46%.

Example 26: Synthesis of N-(2-(1-(1-phenyl-1H-pyrazolo[3,4-d]pyrimidin-4-yl)piperidin-4-yl)ethyl)sulfamide

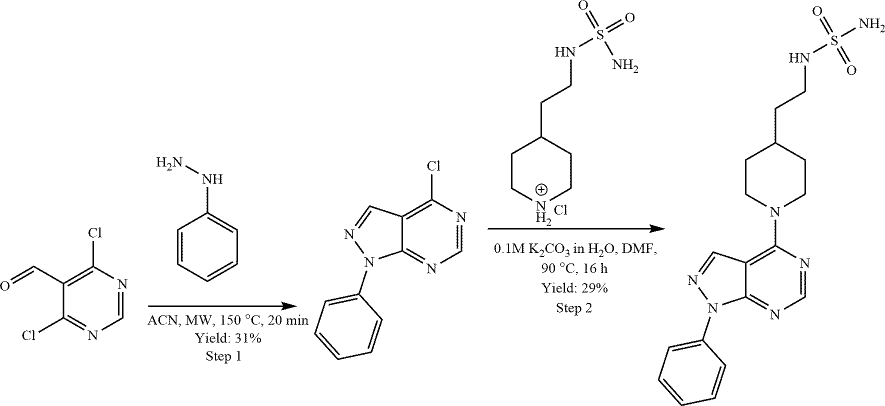

### Step 1: Synthesis of 4-Chloro-l-Phenyl-lH-Pyrazolo[3,4-d]Pyrimidine

Procedure: To a stirred solution of 4,6-dichloropyrimidine-5-carbaldehyde (0.1 g, 0.56 mmol) in ACN (3 mL) was added phenyl hydrazine (0.061 g, 0.56 mmol). Reaction mixture was stirred for 20 mins in microwave at 150° C. Progress of the reaction was monitored by TLC. Reaction mixture was evaporated under reduced pressure. The crude residue was purified by combiflash using 5% ethyl acetate in hexane to afford 4-chloro-1-phenyl-1H-pyrazolo[3,4-d]pyrimidine as white solid (0.04 g, 31%). LC-MS (ES) m/z =231.04 [M+H]⁺.

### Step 2: Synthesis of N-(2-(1-(1-Phenyl-1H-Pyrazolo[3,4-d]Pyrimidin-4-yl)Piperidin-4-yl)Ethyl)Sulfamide

Procedure: To a stirred solution of 4-chloro-1-phenyl-1H-pyrazolo[3,4-d]pyrimidine (0.04 g, 0.17 mmol) in DMF (2 mL) were added N-(2-(piperidin-4-yl)ethyl)sulfamide hydrochloride (0.053 g, 0.19 mmol), 0.1M K₂CO₃ in H₂O (4.3 mL, 0.43 mmol). Reaction mixture was stirred at 90° C. for 16 h and the reaction was monitored by TLC. The reaction mixture was diluted with ethyl acetate (20 mL), washed with water (2 x 10 mL) followed by brine (10 mL), dried over anhydrous sodium sulphate, filtered and evaporated under reduced pressure. The crude residue was purified by combiflash using 70% methanol in dichloromethane to afford N-(2-(1-(1-phenyl-1H-pyrazolo[3,4-d]pyrimidin-4-yl)piperidin-4-yl)ethyl)sulfamide as white solid (0.02 g, 29%). ¹HNMR (400 MHz, DMSO-d₆): δ 8.55 (s, 1 H), 8.34 (s, 1 H), 8.15 (d, J= 8.0 Hz, 2 H), 7.53 (t, J= 7.6 Hz, 2 H), 7.34 (t, J= 6.8 Hz, 1 H),6.44 - 6.40 (m, 3 H), 4.82 - 4.62 (m, 2 H),3.18 - 3.15 (m, 2 H), 2.92 (q, J= 6.8 Hz, 2 H), 1.85 - 1.79 (m, 3 H), 1.43 (q, J= 6.4 Hz, 2 H), 1.22 - 1.16 (m, 2 H); LC-MS (ES) m/z =402.16 [M+H]^{+;} HPLC purity: 98.67%.

Example 27: Synthesis of N-(2-(1-(1H-pyrazolo[4,3-d]pyrimidin-7-yl)piperidin-4-yl)ethyl)sulfamide

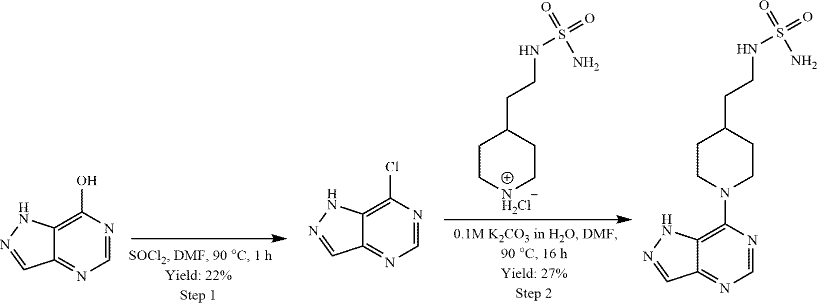

### Step 1: Synthesis of 7-Chloro-1H-Pyrazolo[4,3-d]Pyrimidine

Procedure: To a stirred solution of 1H-pyrazolo[4,3-d]pyrimidin-7-ol (0.2 g, 1.47 mmol) in thionyl chloride (4.2 mL) were added DMF (0.2 mL). Reaction mixture was stirred for 1 h at 90° C. The Progress of the reaction was monitored by TLC. Reaction mixture was diluted with ethyl acetate (50 mL) washed with sat. sodium bicarbonate (30 mL) followed by brine (30 mL), dried over anhydrous sodium sulphate, filtered and evaporated under reduced pressureto afford 7-chloro-1H-pyrazolo[4,3-d]pyrimidine as pale yellow solid (0.05 g, 22%). LC-MS (ES) m/z =168.9 [M+H]⁺.

### Step 2: Synthesis of N-(2-(1-(1H-Pyrazolo[4,3-d]Pyrimidin-7-yl)Piperidin-4-yl)Ethyl)Sulfamide

Procedure: To a stirred solution of 7-chloro-1H-pyrazolo[4,3-d]pyrimidine (0.05 g, 0.32 mmol) in DMF (3 mL) were added N-(2-(piperidin-4-yl)ethyl)sulfamide hydrochloride (0.099 g, 0.35 mmol), 0.1M K₂CO₃ in H₂O (4.8 mL, 0.48 mmol). Reaction mixture was stirred at 90° C. for 16 h and the reaction was monitored by TLC. The reaction mixture was diluted with ethyl acetate (20 mL), washed with water (2 x 10 mL) followed by brine (10 mL), dried over anhydrous sodium sulphate, filtered and evaporated under reduced pressure. The crude residue was purified by combiflash using 15% methanol in dichloromethane to afford N-(2-(1-(1H-pyrazolo[4,3-d]pyrimidin-7-yl)piperidin-4-yl)ethyl)sulfamide as White solid (0.065 g, 32%). ¹HNMR (400 MHz, DMSO-d₆): δ 14.22 (s, 1 H), 8.30 (s, 1 H), 8.14 (s, 1 H), 6.44 - 6.40 (m, 3 H), 3.18 - 3.04 (m, 2 H), 2.91 (q, J = 6.8 Hz, 4 H), 1.80 - 1.77 (m, 3 H), 1.46 - 1.38 (m, 2 H), 1.16 - 1.08 (m, 2 H); LC-MS (ES) m/z = 326.13 [M+H]⁺; HPLC purity: 99.51%.

Example 28: Synthesis of N-(2-(6,7-dimethoxyquinazolin-4-yl)-1,2,3,4-tetrahydroisoquinolin-5-yl)sulfamide

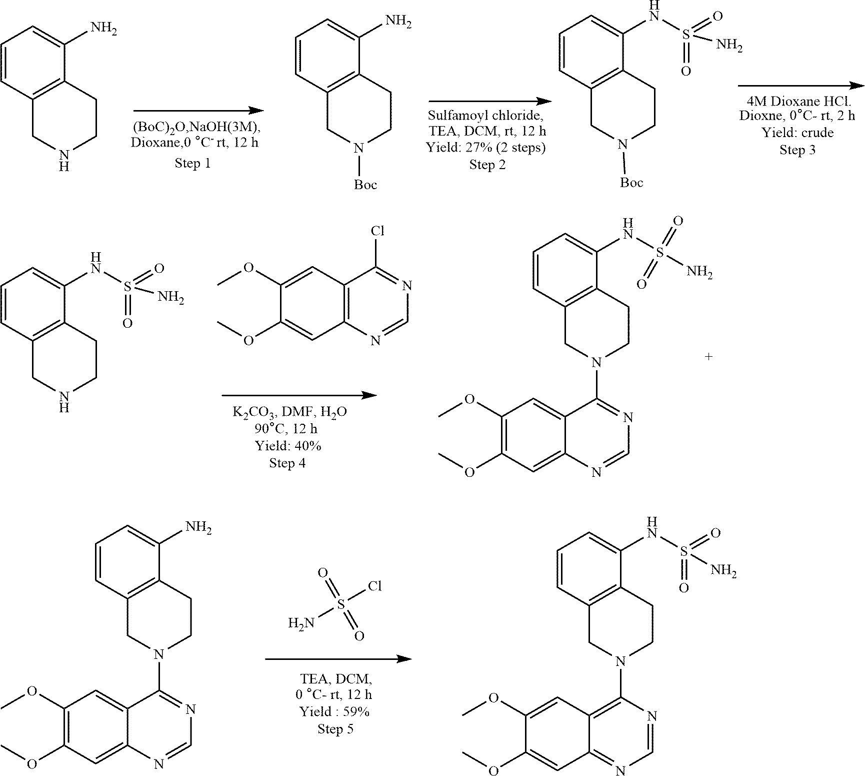

### Step 5: Synthesis of N-(2-(6,7-Dimethoxyquinazolin-4-yl)-1,2,3,4-Tetrahydroisoquinolin-5-yl)Methane Sulfonamide

Procedure: To a stirred solution of 2-(6,7-dimethoxyquinazolin-4-yl)-1,2,3,4-tetrahydroisoquinolin-5-amine (0.06 g, 0.17 mmol) in dichloromethane (10 mL) was added triethyl amine (0.075 mL, 0.53 mmol) followed by sulfamoyl chloride (0.041 g, 0.35 mmol) at 0° C. and allowed the reaction mixture to stir at room temperature for 12 h. The progress of the reaction was monitored by TLC. Then the reaction was diluted with water (100 mL) and extracted with dichloromethane (2 x 50 mL), combined organic layers were washed with brine (50 mL). The organic layer was dried over sodium sulphate, filtered and evaporated under reduced pressure to obtain crude compound. The crude residue was purified by gradient column chromatography using 4% methanol in dichloromethane to afford N-(2-(6,7-dimethoxyquinazolin-4-yl)-1,2,3,4-tetrahydroisoquinolin-5-yl)sulfamide (0.08 g, 59%) as white solid. ¹HNMR (400 MHz, DMSO-d₆): 88.55 (bs, 1 H), 8.52 (s, 1 H),7.29 (d, J = 7.6 Hz, 1 H), 7.21(s, 2 H),7.18 (t, J= 8 Hz, 1 H), 7.18 (d, J= 7.6 Hz, 1H), 6.90 (s, 2 H), 4.77 (s, 2 H), 3.93 (s, 3 H), 3.92 (s, 3 H), 3.87 (t, J= 5.6 Hz, 2 H), 3.13 (t, J= 5.2 Hz, 2 H).LC-MS (ES) m/z = 416.1 [M+H]⁺. HPLC purity 99.66%.

Example 29: 8-(6,7-dimethoxyquinazolin-4-+yl)-2,8-diazaspiro[4.5]decane-2-sulfonamide

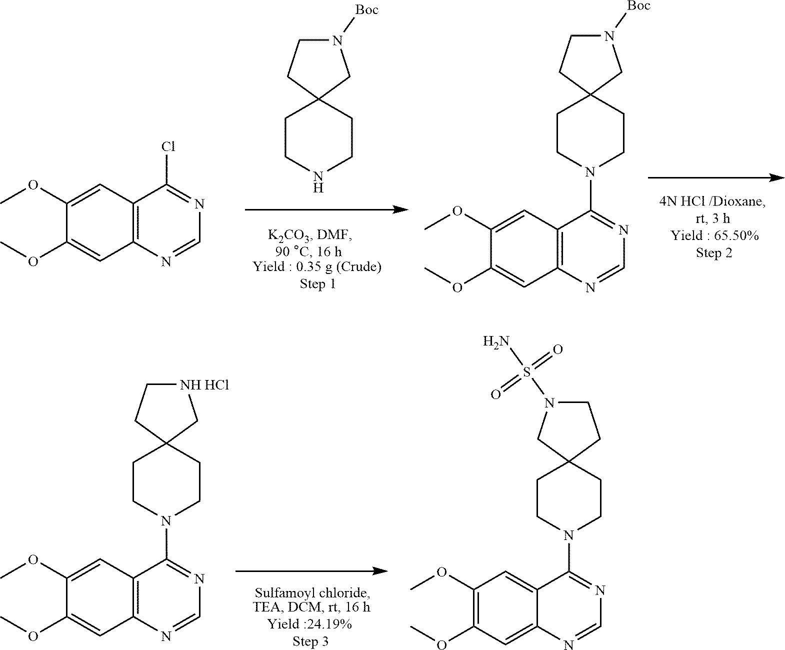

### Step 1: Synthesis of Tert-Butyl 8-(6,7-Dimethoxyquinazolin-4-yl)-2,8-Diazaspiro[4.5]Decane-2-Carboxylate

Procedure: To a stirred solution of 4-chloro-6,7-dimethoxyquinazoline (0.2 g, 0.890 mmol) in DMF (10 mL) was added K₂CO₃ (0.368 g, 2.666 mmol) followed by tert-butyl 2,8-diazaspiro[4.5]decane-2-carboxylate hydrochloride (0.235 g, 0.977 mmol). Reaction mixture was stirred for 16 h at 90° C. Progress of the reaction was monitored by TLC. Reaction mixture was diluted with water (20 mL), extracted with ethyl acetate (2 x 50 mL). The combined organic layer was washed with brine (10 mL), dried over anhydrous sodium sulphate, filtered and evaporated under reduced pressure. The crude residue was washed with diethyl ether to afford tert-butyl 8-(6,7-dimethoxyquinazolin-4-yl)-2,8-diazaspiro[4.5]decane-2-carboxylate as off-white solid, (0.35 g, crude).¹HNMR (400 MHz, DMSO-d₆): 8 8.49 (s, 1 H), 7.93 (s, 2 H), 7.18 (s, 1 H), 7.10 (s, 1 H), 3.98 (d, J= 8.0 Hz, 6 H), 3.57 (s, 3 H), 3.27 (t, J = 11.6 Hz, 2 H), 3.16 (s, 2 H), 1.78 (s, 2 H), 1.68 (t, J = 4.8 Hz, 4 H), 1.38 (s, 9 H). LC-MS (ES) m/z = 430 [M+H]⁺.

### Step 2: Synthesis of 8-(6,7-Dimethoxyquinazolin-4-yl)-2,8-Diazaspiro[4.5]Decane Hydrochloride

Procedure: To a solution of tert-butyl 8-(6,7-dimethoxyquinazolin-4-yl)-2,8-diazaspiro[4.5]decane-2-carboxylate (0.3 g, 6.493 mmol) in dioxane (10.0 ml), then was added 4N HCl in dioxane (5 mL,48.39 mmol). The reaction mixture was stirred at room temperature for 3 h, the reaction mixture concentrated under reduced pressure to get desired crude compound as a white solid (0.15 g, 65.50 %). ¹HNMR (400 MHz, DMSO-d₆): 8 9.34 (bs,2 H),8.74(s1H),7.30(d, J= 8.4 Hz, 2 H), 4.11 (d, J = 13.6 Hz, 2 H), 4.04 (s,2 H), 3.95(d, J = 12.8 Hz, 6 H), 3.26 (t, J = 5.6 Hz, 2 H), 3.07 (s, 2 H), 1.91 (t, J = 7.6 Hz, 2 H), 1.78 (s, 4 H). LC-MS (ES) m/z = 329 [M-H]⁻.

### Step 3: Synthesis of 8-(6,7-Dimethoxyquinazolin-4-yl)-2,8-Diazaspiro[4.5]Decane-2-Sulfonamide

Procedure: To a stirred solution of 8-(6,7-dimethoxyquinazolin-4-yl)-2,8-diazaspiro[4.5]decane hydrochloride (0.15 g, 0.457 mmol) in dichloromethane (20 mL) was added and triethylamine (0.3 mL, 2.970 mmol) followed by sulfamoyl chloride (0.140 g, 1.217 mmol). Reaction mixture was stirred at room temperature for 16 h, reaction was monitored by TLC. The reaction mixture was diluted with water (50 mL), extracted with dichloromethane (2 x 50 mL). The combined organic layer was washed with brine (10 mL), dried over anhydrous sodium sulphate, filtered and evaporated under reduced pressure. The crude residue was purified by combiflash using methanol in dichloromethane to afford 8-(6,7-dimethoxyquinazolin-4-yl)-2,8-diazaspiro[4.5]decane-2-sulfonamide as off white solid (0.065 g, 18.5%). ¹HNMR (400 MHz, DMSO-d₆): 8 8.50 (s, 1 H), 7.18 (s, 1 H), 7.10 (s, 1 H), 6.71 (s, 2 H), 3.89 (d, J = 7.2 Hz, 6 H), 3.58-3.63 (m, 4 H), 3.21 (t, J = 7.2 Hz, 2 H), 3.05 (s, 2 H), 1.71-1.76 (m, 4 H), 1.21 (s, 1 H). HPLC purity 99.90%. LC-MS (ES) m/z = 407[M+H]⁺.

Example 30: 7-(6,7-dimethoxyquinazolin-4-yl)-2,7-diazaspiro[3.5]nonane-2-sulfonamide

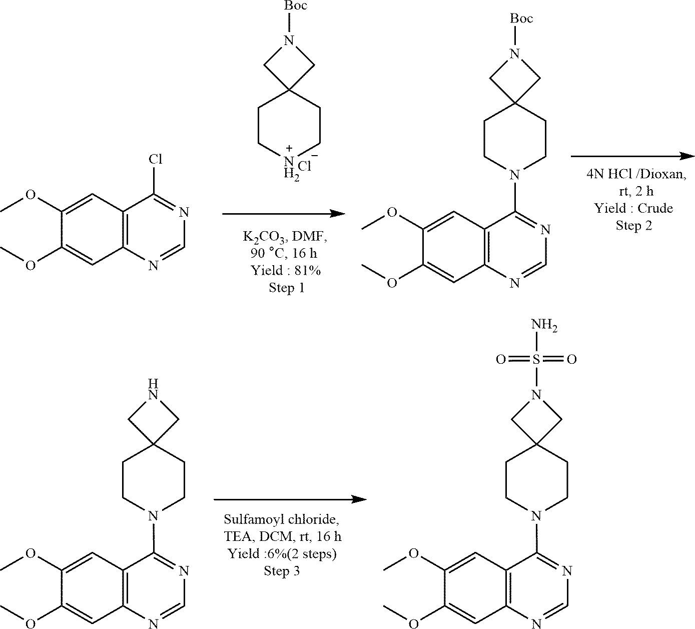

### Step 1: Synthesis of Tert-Butyl 7-(6,7-Dimethoxyquinazolin-4-yl)-2,7-Diazaspiro[3.5]Nonane-2-Carboxylate

Procedure: To a stirred solution of 4-chloro-6,7-dimethoxyquinazoline (0.2 g, 0.89 mmol) in DMF (10 mL) was added K₂CO₃ (0.184 g, 1.33 mmol) followed by 2-(tert-butoxycarbonyl)-2,7-diazaspiro[3.5]nonan-7-ium hydrochloride (0.221 g, 0.979 mmol). Reaction mixture was stirred for 16 h at 90° C. Progress of the reaction was monitored by TLC. Reaction mixture was diluted with water (20 mL), extracted with ethyl acetate (2 x 50 mL). The combined organic layer was washed with brine (10mL), dried over anhydrous sodium sulphate, filtered and evaporated under reduced pressure. The crude residue was purified by combiflash using ethyl acetate in hexaneto afford tert-butyl 7-(6,7-dimethoxyquinazolin-4-yl)-2,7-diazaspiro[3.5]nonane-2-carboxylate (0.3 g, 81%) as an off white solid. LC-MS (ES) m/z =415.2 [M+H]⁺.

### Step 2: Synthesis of 7-(6,7-Dimethoxyquinazolin-4-yl)-2,7-Diazaspiro[3.5]Nonan-2-Ium Chloride

Procedure: A single neck flask was charged with tert-butyl 7-(6,7-dimethoxyquinazolin-4-yl)-2,7-diazaspiro[3.5]nonane-2-carboxylate (0.3 g, 0.724 mmol) and 4N HCl in dioxane (3 mL). The reaction mixture was stirred at room temperature for 2 h, then the reaction mixture concentrated under reduced pressure to afford 7-(6,7-dimethoxyquinazolin-4-yl)-2,7-diazaspiro[3.5]nonan-2-ium chloride (0.25 g crude) as an white solid. LC-MS (ES) m/z = 315.2 [M+H]⁺.

### Step 3: Synthesis of 7-(6,7-Dimethoxyquinazolin-4-yl)-2,7-Diazaspiro[3.5]Nonane-2-Sulfonamide

Procedure: To a stirred solution of 7-(6,7-dimethoxyquinazolin-4-yl)-2,7-diazaspiro[3.5]nonan-2-ium chloride (0.25 g crude, 0.796 mmol) in dichloromethane (20 mL) was added and triethylamine (0.44 mL, 3.18 mmol) followed by sulfamoyl chloride (0.275 g, 2.38 mmol). Reaction mixture was stirred at room temperature for 16 h, reaction was monitored by TLC. The reaction mixture was diluted with water (50 mL), extracted with dichloromethane (2 x 50 mL). The combined organic layer was dried over anhydrous sodium sulphate, filtered and evaporated under reduced pressure. The crude residue was purified by combiflash using methanol in dichloromethane as eluent. The obtained solid was washed with acetonitrile and diethyl ether twice to afford 7-(6,7-dimethoxyquinazolin-4-yl)-2,7-diazaspiro[3.5]nonane-2-sulfonamide [0.02 g, 6% (2 steps)]as an off-white solid. ¹HNMR (400 MHz, DMSO-d₆):8.50 (s, 1 H),7.19 (s, 1 H),7.09 (s, 1 H), 6.87 (s, 2 H), 3.90 (s, 3 H), 3.89 (s, 3 H), 3.54-3.51 (m, 8 H),3.54-3.51(m, 8 H), 1.88 (m, 4 H). LC-MS m/z calcd for [M+H]⁺394.1, HPLC purity 98.23%.

Example 31: Synthesis of 2-(6,7-dimethoxyquinazolin-4-yl)-1,2,3,4-tetrahydroisoquinoline-5-sulfonamide

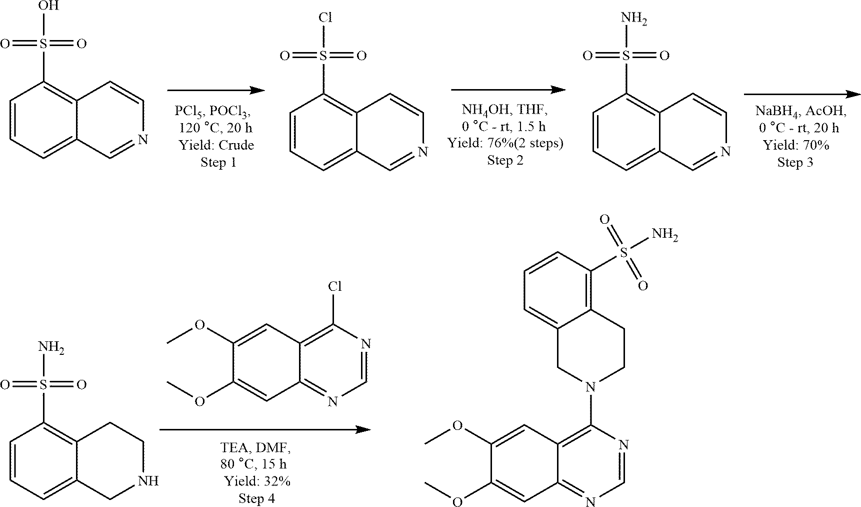

### Step 1: Synthesis of Isoquinoline-5-Sulfonyl Chloride

Procedure: To a stirred solution of compound isoquinoline-5-sulfonic acid (2 g, 9.559 mmol) in POCl₃ (15 mL) was added PC1₅ (2.98 g, 14.338 mmol). The resulting mixture was stirred at 120° C. for 20 h. The progress of the reaction was monitored by TLC. Then the reaction was quenched with dichloromethane (100 mL) and stirred for 15 min. The solid formed was filtered and the residue was washed with dichloromethane, dried under reduced pressure to get title compound as pale yellow solid (1.2 g, Crude). LC-MS m/z = 228.0 [M+H]⁺ .

### Step 2: Isoquinoline-5-Sulfonamide

Procedure: To a stirred solution of compound isoquinoline-5-sulfonyl chloride (0.5 g, 2.192 mmol) in tetrahydrofuran (10 mL) at 0° C. was added ammonium hydroxide solution (0.26 mL, 6.578 mmol) and stirred for 1.5 h at room temperature. The progress of the reaction was monitored by TLC. Then the reaction was quenched with water (10 mL) and extracted with 5% methanol in dichloromethane (3 x 50 mL), combined organic layers were washed with water (10 mL) and brine (10 mL). The organic layer was dried over sodium sulphate, filtered and evaporated under reduced pressure, purified by combiflash purifier using 8% methanol in dichloromethane to afford isoquinoline-5-sulfonamide (0.35 g, 76% (2 Steps)) as off-white solid. ¹HNMR (400 MHz, DMSO-d₆): δ 9.45 (s, 1 H), 8.66 (d, J=5.6 Hz, 1 H), 8.42-8.32 (m, 3 H), 7.82-7.78 (m, 1 H), 7.74 (s, 2 H). LC-MS m/z = 209.1 [M+H]⁺.

### Step 3: 1,2,3,4-Tetrahydroisoquinoline-5-Sulfonamide

Procedure: To a stirred solution of isoquinoline-5-sulfonamide (0.35 g, 1.680 mmol) in glacial acetic acid (3 mL) at 0° C. was added sodium borohydride (0.31 g, 8.403 mmol). The reaction mixture was stirred at room temperature for 20 h. The progress of the reaction was monitored by TLC, then the reaction mixture was quenched with ammonium hydroxide solution (pH =7), diluted with water (10 mL) and extracted with dichloromethane (3 x 50 mL), combined organic layers were washed with water (20 mL) followed by brine (10 mL), organic layers were dried over anhydrous sodium sulfate, filtered and evaporated under reduced pressure and washed with n-Pentane, diethylether to get 1,2,3,4-tetrahydroisoquinoline-5-sulfonamide (0.25 g, 70.2%) as off white solid. ¹HNMR (400 MHz, DMSO-d₆): δ 7.67 (d, J =7.6 Hz, 1 H), 7.30-7.21 (m, 4 H), 3.89 (s, 1 H), 3.03-3.02 (m, 2 H), 2.95-2.92 (m, 2 H), 1.08 (t, J = 6.8 Hz, 1 H). LC-MS m/z = 213.1 [M+H]⁺.

### Step 4: Synthesis of Compound 2-(6,7-Dimethoxyquinazolin-4-yl)-1,2,3,4-Tetrahydroisoquinoline-5-Sulfonamide

Procedure: To a stirred solution of compound 1,2,3,4-tetrahydroisoquinoline-5-sulfonamide (0.15 g, 0.706 mmol) in DMF (3 mL) were added triethylamine (0.29 mL, 2.119 mmol) and 4-chloro-6,7-dimethoxyquinazoline (0.174 g, 0.777 mmol) at room temperature. The reaction mixture was stirred at 80° C. 15 h. The progress of the reaction was monitored by TLC. Then the reaction was quenched with water (10 mL) and extracted with 5% methanol in dichloromethane (3 x 50 mL), combined organic layers were washed with water (10 mL) and brine (10 mL). The organic layer was dried over anhydrous sodium sulphate, filtered and evaporated under reduced pressure, purified by combiflash purifier using 12% methanol in dichloromethane to afford 2-(6,7-dimethoxyquinazolin-4-yl)-1,2,3,4-tetrahydroisoquinoline-5-sulfonamide (0.09 g, 32 %) as off white solid. ¹HNMR (400 MHz, DMSO-d₆): δ 8.53 (s, 1 H), 7.77 (d, J = 7.6 Hz, 1 H), 7.51 (d, J = 7.2 Hz, 1 H), 7.43 (bs, 2 H), 7.39-7.35 (m, 1 H), 7.23 (d, J = 8.0 Hz, 2 H), 4.89 (s, 2 H), 3.94-3.92 (m, 8 H), 3.45-3.44 (m, 2 H). m/z = 401.1[M+H]⁺, HPLC purity = 99.65% at 254 nm.

Example 32: 7-(6,7-dimethoxyquinazolin-4-yl)-3,4-dihydroisoquinoline-2(1H)-sulfonamide

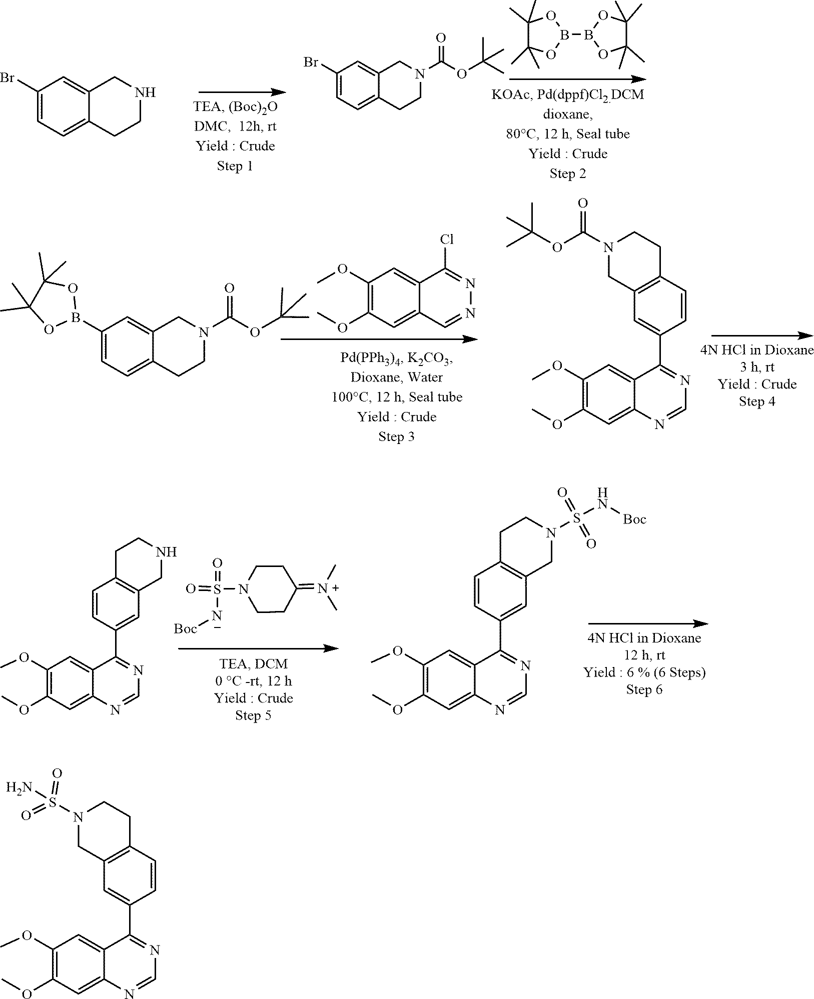

### Step 2: Synthesis of Tert-Butyl 7-(4,4,5,5-Tetramethyl-1,3,2-Dioxaborolan-2-yl)-3,4-Dihydroisoquinoline-2(1H)-Carboxylate

Procedure: A solution of tert-butyl 7-bromo-3,4-dihydroisoquinoline-2(1H)-carboxylate (1.0 g, 3.2029 mmol) in dioxane (30 mL) was added potassium acetate (0.943 g, 9.6089 mmol) and 4,4,4′,4′,5,5,5′,5′-octamethyl-2,2′-bi(1,3,2-dioxaborolane) (0.976 g, 3.8435 mmol). The resulting mixture was purged with argon gas for 15 minutes. [1,1‘-Bis(diphenylphosphino)ferrocene]dichloropalladium(II), complex with dichloromethane (0.13 g, 0.1601 mmol) was added into reaction mixture and again purged for 5 minutes and allowed to stir in sealed tube at 80° C. for 12 h. The progress of reaction was monitored by TLC. The reaction mixture was cooled to room temperature, concentrated under reduced pressure. The crude residue was purified by combiflash using ethyl acetate in n-hexane to afford tert-butyl 7-(4,4,5,5-tetramethyl-1,3,2-dioxaborolan-2-yl)-3,4-dihydroisoquinoline-2(1H)-carboxylate as colourless liquid (1.1 g impure). LC-MS m/z calcd for [M+H]⁺358.23, found 258.2.

### Step 3: Synthesis of Tert-Butyl 7-(6,7-Dimethoxyquinazolin-4-yl)-3,4-Dihydroisoquinoline-2(1H)-Carboxylate

Procedure: To the solution of tert-butyl 7-(4,4,5,5-tetramethyl-1,3,2-dioxaborolan-2-yl)-3,4-dihydroisoquinoline-2(1H)-carboxylate (1.0 g, 2.7834 mmol) in dioxane (25 mL) and water (2.5 mL) was added 4-chloro-6,7-dimethoxyquinazoline (0.5 g, 2.2267 mmol) and potassium carbonate (1.15 g , 8.3502 mmol). The resulting mixture was purged by argon gas for 15 minutes. Tetrakis(triphenylphosphane)palladium(0) (0.16 g, 0.1391 mmol) was added into reaction mixture and again purged for 5 minutes and allowed to stir in sealed tube at 100° C. for 12 h. The progress of reaction was monitored by TLC. The reaction mixture was quenched with water, extracted with ethyl acetate, washed with water and brine. The organic layer was dried over sodium sulphate, filtered and evaporated under reduced pressure. The crude residue was purified by combiflash using ethyl acetate in n-hexane to afford tert-butyl 7-(6,7-dimethoxyquinazolin-4-yl)-3,4-dihydroisoquinoline-2(1H)-carboxylateas white solid (0.89 g impure). LC-MS m/z calcd for [M+H]⁺ 422.20, found 422.4

### Step 4: Synthesis of 6,7-Dimethoxy-4-(1,2,3,4-Tetrahydroisoquinolin-7-yl)Quinazoline

Procedure: The compound tert-butyl 7-(6,7-dimethoxyquinazolin-4-yl)-3,4-dihydroisoquinoline-2(1H)-carboxylate (0.5 g, 1.1862 mmol) in round bottom flask at 0° C. was added 4 M HCl in dioxane (15 mL) and reaction mixture stirred at room temperature for 3 h. The progress of the reaction was monitored by TLC. The reaction mixture evaporated under reduced pressure. The crude residue was purified by combiflash using methanol in dichloromethane to afford 6,7-dimethoxy-4-(1,2,3,4-tetrahydroisoquinolin-7-yl)quinazoline (0.39 g impure) as yellow solid. LC-MS m/z calcd for [M+H] ⁺322.15, found 322.3.

### Step 5: Synthesis of Tert-Butyl ((7-(6,7-Dimethoxyquinazolin-4-yl)-3,4-Dihydroisoquinolin-2(1H)-yl)Sulfonyl)Carbamate

Procedure: To a stirred solution of 6,7-dimethoxy-4-(1,2,3,4-tetrahydroisoquinolin-7-yl)quinazoline (0.25 g, 0.7776 mmol) in dichloromethane (25 mL) was added triethylamine (0.32 mL, 2.3328 mmol )at 0° C. and stirred for 15 minutes.(tert-butoxycarbonyl)((4-(dimethyliminio)pyridin-1(4H)-yl)sulfonyl)amide (0.234 g, 0.7776 mmol) was added into reaction mixture at 0° C. The reaction mixture was stirred at room temperature for 12 h. The progress of the reaction was monitored by TLC. The reaction mixture was diluted with dichloromethane and washed with water followed by brine solution, dried over anhydrous sodium sulphate, evaporated under reduced pressure. The crude residue was purified by combiflash using methanol in dichloromethane to afford tert-butyl ((7-(6,7-dimethoxyquinazolin-4-yl)-3,4-dihydroisoquinolin-2(1H)-yl)sulfonyl)carbamate (0.229 g impure) as white solid.LC-MS m/z calcd for [M+H]⁺501.18, found 499.3.

### Step 6: Synthesis of 7-(6,7-Dimethoxyquinazolin-4-yl)-3,4-Dihydroisoquinoline-2(1H)-Sulfonamide

Procedure: The compound tert-butyl ((7-(6,7-dimethoxyquinazolin-4-yl)-3,4-dihydroisoquinolin-2(1H)-yl)sulfonyl)carbamate(0.15 g, 0.2997 mmol) in round bottom flask at 0° C. was added 4 M HCl in dioxane (25 mL) and reaction mixture stirred at room temperature for 12 h. The progress of the reaction was monitored by TLC. The reaction mixture evaporated under reduced pressure. The crude residue was purified by combiflash using methanol in dichloromethane to afford 7-(6, 7-dimethoxyquinazolin-4-yl)-3,4-dihydroisoquinoline-2(1H)-sulfonamide (0.145 g, 6% (6 steps yield)) as off white solid. ¹HNMR (400 MHz, DMSO-d₆) : 8 9.09 (s, 1 H), 7.63 (d, J= 7.2 Hz, 2 H), 7.42 (s, 1 H), 7.38 (d, J= 7.6 Hz, 1 H), 7.32 (s, 1 H), 6.92 (s, 2 H), 4.32 (s, 2 H),3.99 (s, 3 H),3.83 (s, 3 H), 3.35-3.32 (m, 2 H), 3.03 (d, J= 5.2 Hz, 2 H), LC-MS m/z calcd for [M+H]⁺401.12, found 401.1.

Example 33: N-(3-(2-((6,7-dimethoxyquinazolin-4-yl)amino)ethyl)phenyl)sulfamide

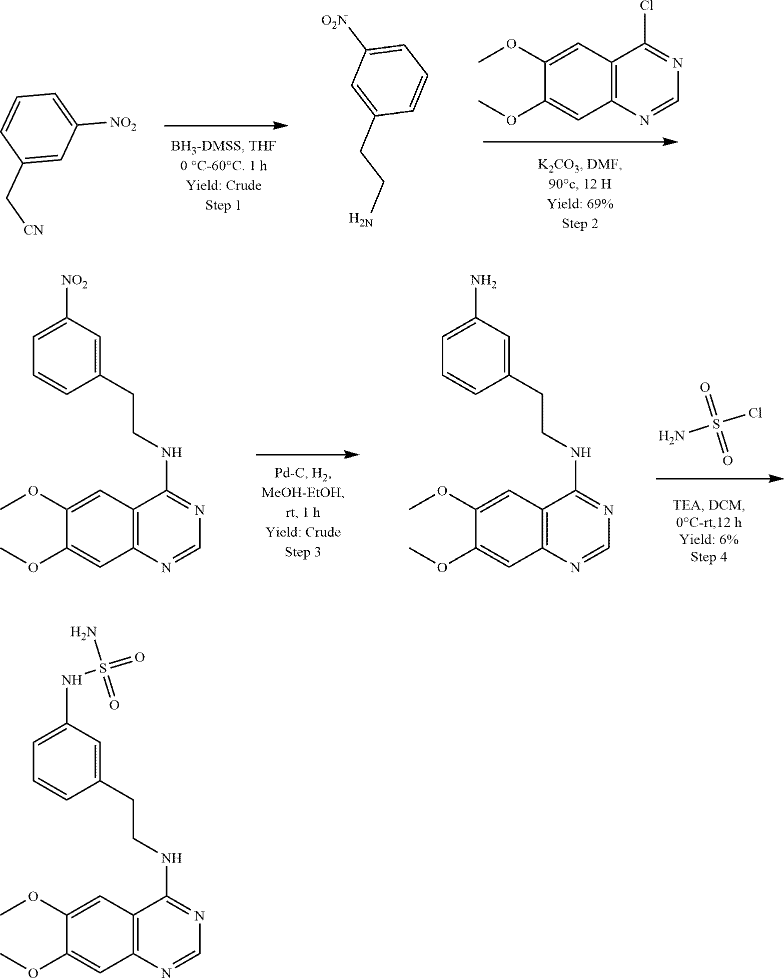

### Step 1: 2-(3-Nitrophenyl)Ethan-1-Amine Hydrochloride

Procedure: To a stirred solution of 2-(3-nitrophenyl)acetonitrile (2 g, 12.3 mmol) in THF (20 mL) was added borane dimethyl sulphide complex (12.3 mL, 24.6 mmol) dropwise at 0° C. and the resulting reaction mixture was heated at 60° C. for 1 h. The completion of the reaction was monitored by TLC using 50% EtOAc in n-hexane as eluent. Then the reaction mixture was quenched with methanol at 0° C. until the evolution of effervescence stops and the reaction mixture was evaporated under reduced pressure. The residue obtained was dissolved in methanol (25 mL) then added 4 M HCl in dioxane (2 mL) and heated at 65° C. for 30 minutes. After reaction mixture was evaporated under reduced pressure. The solid obtained was triturated with diethyl ether (2*20 mL), dried under vacuum pressure to 2-(3-nitrophenyl)ethan-1-amine hydrochloride as off-white solid (2 g, crude). LC-MS (ES) m/z = 167.1 [M+H]⁺.

### Step 2: 6,7-Dimethoxy-N-(3-Nitrophenethyl)Quinazolin-4-Amine

Procedure: To a stirred solution of2-(3-nitrophenyl)ethan-1-amine hydrochloride (0.5 g, 2.40 mmol) in DMF (12 mL) was added potassium carbonate (0.66 g, 4.80 mmol) in water (2.5 mL) at room temperature followed by added 4-chloro-6,7-dimethoxyquinazoline (0.60 g, 2.70 mmol) and the resulting reaction mixture was heated at 90° C. for 12 h. After completion of the reaction monitored by TLC using 5% methanol in DCM, to reaction mixture was added water (20 mL) and extracted with ethyl acetate (2*50 mL) and separated the organic layer. The combined organic layer was washed with brine (30 mL), dried over sodium sulphate, filtered and evaporated under reduced pressure. The crude obtained was purified by combiflash purifier using 5% MeOH in DCM to afford 6,7-dimethoxy-N-(3-nitrophenethyl)quinazolin-4-amine as yellow solid (0.6 g, 68%). ¹HNMR (400 MHz, DMSO-d₆): δ 8.33 (s, 1 H), 8.12 (s, 1 H), 8.05 (d, J= 8 Hz, 1 H), 7.94-8.0(m, 1 H), 7.71(d, J= 7.6 Hz, 1 H), 7.56 (t, J= 8 Hz, 1 H), 7.51 (s, 1 H), 7.06 (s, 1 H), 3.87 (s, 3 H), 3.84 (s, 3 H), 3.79 (q, J= 6.4 Hz, 2 H), 3.10 (t, J= 7.2 Hz, 2 H). LC-MS (ES) m/z = 355.1 [M+H]⁺.

### Step 3: Benzyl N-(3-Aminophenethyl)-6,7-Dimethoxyquinazolin-4-Amine

Procedure: To a stirred solution of 6,7-dimethoxy-N-(3-nitrophenethyl)quinazolin-4-amine (0.2 g, 5.60 mmol) in MeOH (10 mL)-EtOH (10 mL) was added 10% palladium on carbon (0.03 g) and the resulting reaction mixture was stirred for 1 h at room temperature under hydrogen atmosphere. After completion of the reaction was monitored by TLC using 5% methanol in dichloromethane as eluent, the reaction mixture was filtered through diatomaceous earth; organic layer was concentrated under reduced pressure to afford the desired crude compound benzyl N-(3-aminophenethyl)-6,7-dimethoxyquinazolin-4-amine as yellow solid. (0.2 g, crude). LC-MS (ES) m/z = 325.2 [M+H]⁺.

### Step 4: N-(3-(2-((6,7-Dimethoxyquinazolin-4-yl)Amino)Ethyl)Phenyl)Sulfamide

Procedure: To a stirred solution of benzyl N-(3-aminophenethyl)-6,7-dimethoxyquinazolin-4-amine (0.2 g, 0.61 mmol) in DCM (10 mL) was added triethylamine (0.25 mL, 1.83 mmol) at 0° C. and the resulting reaction mixture was stirred for 5 minutes at 0° C. followed by added sulfamoyl chloride (0.14 g, 1.20 mmol) and the resulting reaction mixture was stirred for 12 h at room temperature. The completion of the reaction was monitored by TLC using 5% methanol in dichloromethane as eluent. Then the reaction mixture was quenched with water (10 mL) and extracted with ethyl acetate (2*30 mL). The combined organic layer was washed with brine (20 mL), dried over sodium sulphate, filtered and evaporated under reduced pressure. The obtained crude was purified by combiflash purifier using 5% methanol in dichloromethane as eluent to afford N-(3-(2-((6,7-dimethoxyquinazolin-4-yl)amino)ethyl)phenyl)sulfamide (0.016 g, 6.4%). ¹HNMR (400 MHz, DMSO-d₆): δ 9.38 (s, 1 H), 8.38 (s, 1 H), 8.12 (bs, 1 H), 7.56 (s, 1 H), 7.18 (t, J= 8 Hz, 1 H), 7.07 (s, 2 H), 7.0-7.04 (m, 3 H), 6.87 (d, J= 7.6 Hz, 1 H), 3.88 (s, 3 H), 3.86 (s, 3 H), 3.70 (q, J= 6.4 Hz, 2 H), 2.89 (t, J= 7.6 Hz, 2 H). LC-MS (ES) m/z = 404.1 [M+H]⁺.

Example 34: Synthesis of N-(4-(((6,7-dimethoxyquinazolin-4-yl)amino)methyl)phenyl) sulfamidetrifluoroacetate:

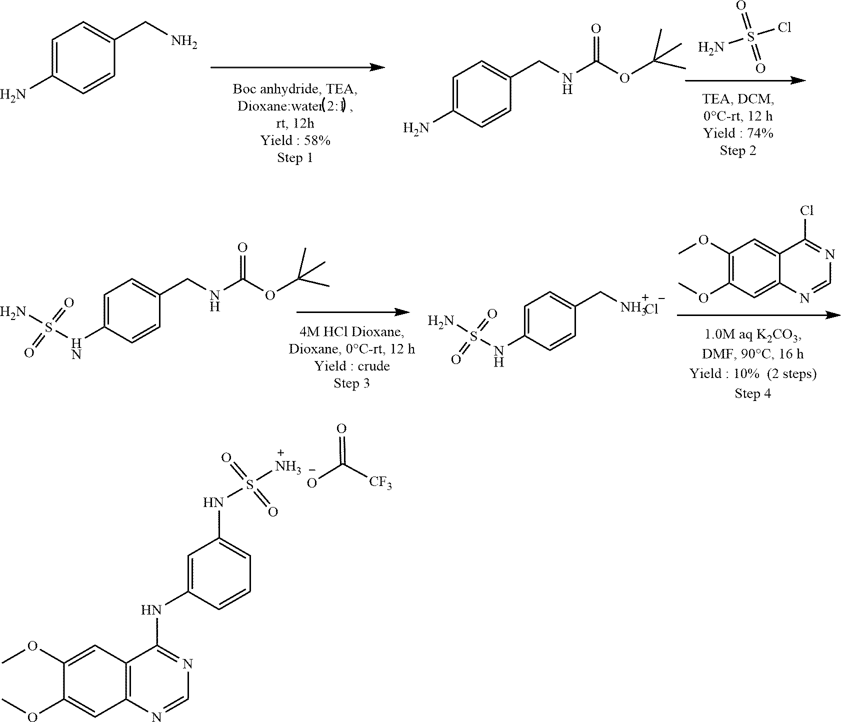

### Step 1: Synthesis of Tert-Butyl (4-Aminobenzyl)Carbamate

Procedure: To a stirred solution of 4-(aminomethyl)aniline (2 g, 16.37 mmol) in 1,4-dioxane(30 mL)and water(15 mL) was added triethyl amine (2.74 mL, 19.64 mmol) followed by Boc anhydride (3.95 g, 18.00 mmol) at 0° C. and allowed the reaction mixture to stir at room temperature for 12 h. The progress of the reaction was monitored by TLC. Evaporated the solvent under reduced pressure and diluted with water (40 mL), extracted with ethyl acetate (2 x 150 mL). The combined organic layer ware washed with brine (40 mL), dried over anhydrous sodium sulphate, filtered and evaporated under reduced pressure to obtain crude compound. The crude residue was purified by combiflash using ethyl acetate in hexane to afford tert-butyl (4-aminobenzyl)carbamate (2.1 g, 58 %) as light yellow solid. LC-MS (ES) m/z = 167.1 [M-55]⁺.

### Step 2: Synthesis of Tert-Butyl (4-(Sulfamoylamino)Benzyl)Carbamate

Procedure: To a stirred solution of tert-butyl (4-aminobenzyl)carbamate (0.5 g, 2.24 mmol) in dichloromethane(15 mL) and was added triethyl amine (0.94 mL, 6.74 mmol) followed by sulfamoyl chloride (0.52 g, 4.49 mmol) at 0° C. and allowed the reaction mixture to stir at room temperature for 12 h. The progress of the reaction was monitored by TLC. Evaporated the solvent under reduced pressure and diluted with water (20 mL), extracted with dichloromethane (2 x 40 mL). The combined organic layer ware washed with brine (20 mL), dried over anhydrous sodium sulphate, filtered and evaporated under reduced pressure to obtain crude compound. The crude residue was purified by combiflash using ethyl acetate in hexane to afford tert-butyl (4-(sulfamoylamino)benzyl)carbamate (0.5 g , 74 %) as off white solid. LC-MS (ES) m/z = 300.1 [M-H]⁺.

### Step 3: Synthesis of N-(4-(Aminomethyl)Phenyl)Sulfamide Hydrochloride

Procedure: To a stirred solution of tert-butyl (4-(sulfamoylamino)benzyl)carbamate(0.5 g, 1.65 mmol) in dioxane (15 mL)and was added 4 M HCl in dioxane(5 mL) at 0° C. and allowed the reaction mixture to stir at room temperature for 12 h. The progress of the reaction was monitored by TLC. Evaporated the solvent under reduced pressure to obtain crude compound as (0.45 g, crude) as brown solid. The crude residue was carried out for next step without purification. LC-MS (ES) m/z = 200.0 [M-H]⁺.

### Step 4: Synthesis of N-(4-(((6,7-Dimethoxyquinazolin-4-yl)Amino)Methyl)Phenyl) Sulfamidetrifluoroacetate

Procedure: To a stirred solution of N-(4-(aminomethyl)phenyl)sulfamide hydrochloride (0.3 g, 1.26 mmol) and 4-chloro-6,7-dimethoxyquinazoline (0.42 g, 1.89 mmol) in dimethyl formamide (8 mL)was added 1 M aqueous potassium carbonate (3.8 mL, 3.78 mmol) and heated the reaction mixture to 90° C. for 16 h. Progress of the reaction was monitored by TLC. The reaction mixture was diluted with water (20 mL), extracted with dichloromethane (2 x 40 mL). The combined organic layer ware washed with brine (20 mL), dried over anhydrous sodium sulphate, filtered and evaporated under reduced pressure to obtain crude compound. The crude residue was purified by prep-HPLC. Conditions: Column: Intersil ODS 3 V(250 mmx4.6 mmx5 mic), Mobile phase(A): 0.1% TFA in water Mobile phase(B): ACN: Flow rate: 1.0 mL/min to afford N-(4-(((6,7-dimethoxyquinazolin-4-yl)amino)methyl)phenyl)sulfamide trifluoroacetate (0.12 g, 10%) as white solid. ¹HNMR (400 MHz, DMSO-d₆): δ 14.2 (bs, 1 H), 9.95 (s, 1 H), 9.45 (s, 1 H), 8.77 (s, 1 H), 7.86 (s, 1 H), 7.29 (d, J = 8.4 Hz, 2H), 7.18 (s, 1H), 7.12 (d, J = 8.4 Hz, 2 H), 7.02 (s, 2 H), 4.84 (d, J = 5.2 Hz, 2 H), 3.94 (s, 3 H), 3.91 (s, 3H). LC-MS (ES) m/z = 390.1 [M+H]⁺. HPLC purity 99.66%.

Example 35: Synthesis of N-(2-(1-(quinazolin-4-yl)piperidin-4-yl)ethyl)sulfamide

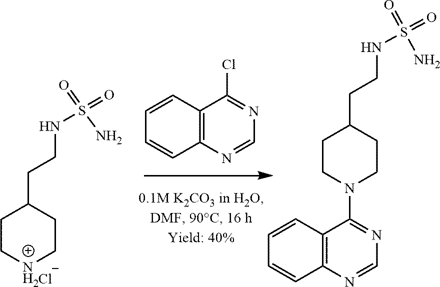

Procedure: To a stirred solution of 4-chloroquinazoline (0.1 g, 0.609 mmol) in DMF (3 mL) were added N-(2-(piperidin-4-yl)ethyl)sulfamide hydrochloride(0.18 g, 0.67 mmol), 0.1M K₂CO₃ in H₂O (9.1 mL, 0.91 mmol). The reaction mixture was stirred at 90° C. for 16 h and the reaction was monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 90% ethyl acetate in hexane to afford N-(2-(1-(quinazolin-4-yl)piperidin-4-yl)ethyl)sulfamide as off-white solid (0.08 g, 40%). ¹H NMR (400 MHz, DMSO-d₆): δ 8.57 (s, 1 H), 7.93 (d, J= 7.6 Hz, 1 H), 7.80 (s, 2 H), 7.53 - 7.49 (s, 1 H), 6.44 - 6.40 (m, 3 H), 4.27 (d, J= 13.2 Hz, 2 H), 3.16 - 3.08 (m, 2 H), 2.93 (q, J= 6.8 Hz, 2 H), 1.81 - 1.72 (m, 3 H), 1.47 (q, J= 6.4 Hz, 2 H), 1.37 - 1.22 (m, 2 H); LC-MS (ES) m/z = 336.2 [M+H]⁺; HPLC purity: 99.96%.

Example 36: Synthesis of N-(1-(6-(3,4-dimethoxyphenyl)pyrimidin-4-yl)piperidin-4-yl)sulfamide

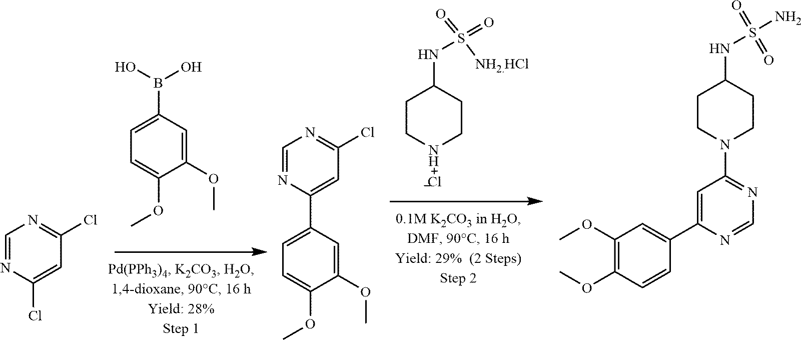

### Step 1: Synthesis of 4-Chloro-6-(3,4-Dimethoxyphenyl)Pyrimidine

Procedure: To a stirred solution of 4,6-dichloropyrimidine (1.0 g, 6.71 mmol) in 1,4-dioxane (10 mL) were added (3,4-dimethoxyphenyl)boronic acid (0.85 g, 4.69 mmol), Potassium carbonate (1.25 g, 9.06 mmol, in 14 mL H₂O) resulting mixture was degassed for 15 mins with argon and added Pd(PPh₃)₄ (0.19 g, 0.16 mmol) degassed for another 10 mins. Resulting mixture was stirred for 16 h at 90° C. The progress of the reaction was monitored by TLC. Reaction mixture was filtered through celite bed and the filtrate was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 18% ethyl acetate in hexane to afford 4-chloro-6-(3,4-dimethoxyphenyl)pyrimidine as off-white solid (0.47 g, 28%). LC-MS (ES) m/z =251.05 [M+H]⁺.

### Step 2: Synthesis of N-(1-(6-(3,4-Dimethoxyphenyl)Pyrimidin-4-yl)Piperidin-4-yl)Sulfamide

Procedure: To a stirred solution of 4-chloro-6-(3,4-dimethoxyphenyl)pyrimidine (0.1 g, 0.39 mmol)in DMF (3 mL) were added N-(piperidin-4-ylmethyl)sulfamide hydrochloride (0.099 g, 0.39 mmol), 0.1M K₂CO₃ in H₂O (9.9 mL, 0.99 mmol). Reaction mixture was stirred at 90° C. for 16 h, reaction was monitored by TLC. The reaction mixture was diluted with ethyl acetate (20 mL), washed with water (2 x 10 mL) followed by brine (10 mL), dried over anhydrous sodium sulphate, filtered and evaporated under reduced pressure. The crude residue was purified by combiflash using 60-70% ethyl acetate in hexane to afford N-(1-(6-(3,4-dimethoxyphenyl)pyrimidin-4-yl)piperidin-4-yl)sulfamide as off-white solid (0.05 g, 29% over 2 steps). ¹H NMR (400 MHz, DMSO-d₆): δ 8.50 (s, 1 H), 7.75 (dd, J= 8.4, 2.0 Hz, 1 H), 7.70 (s, 1 H),7.23 (s, 1 H), 7.02 (d, J= 8.4 Hz, 1 H), 6.60 (d, J= 7.6 Hz, 1 H), 6.52 (s, 2 H), 4.38 (d, J= 13.2 Hz, 2 H), 3.83 (s, 3 H), 3.80 (s, 3 H), 3.40 - 3.39 (m, 1 H), 3.09 (t, J= 11.6 Hz, 2 H), 1.94 (d, J= 10.8 Hz, 2 H), 1.43 - 1.38 (m, 2 H); LC-MS (ES) m/z =394.15 [M+H]⁺; HPLC purity 99.56%.

Example 37: Synthesis of N-((1-(6-(3,4-dimethoxyphenyl)pyrimidin-4-yl)piperidin-4-yl)methyl)sulfamide

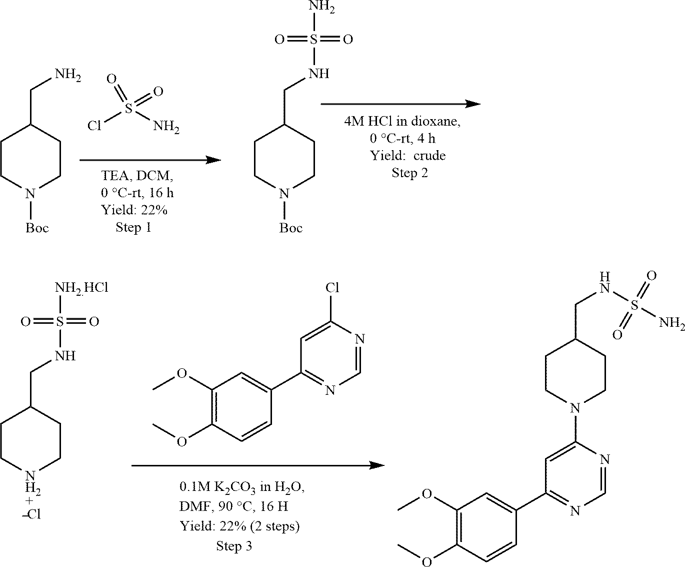

### Step 1: Synthesis of Tert-Butyl 4-((Sulfamoylamino)Methyl)Piperidine-1-Carboxylate

Procedure: To a stirred solution of tert-butyl 4-(aminomethyl)piperidine-1-carboxylate(0.5 g, 2.33 mmol) in dichloromethane (20 mL) were added sulfomyl chloride (0.53 g, 4.67 mmol), triethylamine (0.98 mL, 7.00 mmol). Reaction mixture was stirred at room temperature for 16 h and the reaction was monitored by TLC. The reaction mixture was diluted with dichloromethane (20 mL), washed with water (2 x 10 mL) followed by brine (10 mL), dried over anhydrous sodium sulphate, filtered and evaporated under reduced pressure. The crude residue was purified by combiflash using 50% ethyl acetate in hexaneto afford tert-butyl 4-((sulfamoylamino)methyl)piperidine-1-carboxylateas colorless liquid (0.15 g, 22%). LC-MS (ES) m/z =294.14 [M+H]⁺.

### Step 2: Synthesis of N-(Piperidin-4-Ylmethyl)Sulfamide Hydrochloride

Procedure: To a stirred solution of tert-butyl 4-((sulfamoylamino)methyl)piperidine-1-carboxylate (0.15 g, 0.51 mmol) in 1,4-dioxane (2 mL) was added 4M HCl in 1,4-dioxane (5 mL). Reaction mixture was stirred at room temperature for 4 h and the reaction was monitored by TLC. The reaction mixture was evaporated under reduced pressure and co-distilled with toluene (twice) and dried to get N-(piperidin-4-ylmethyl)sulfamide hydrochloride as pale brown liquid (0.13 g, crude). LC-MS (ES) m/z =194.09 [M+H]⁺.

### Step 3: Synthesis of N-((1-(6-(3,4-Dimethoxyphenyl)Pyrimidin-4-yl)Piperidin-4-yl)Methyl)Sulfamide

Procedure: To a stirred solution of 4-chloro-6-(3,4-dimethoxyphenyl)pyrimidine (0.13 g, 0.49 mmol) in DMF (3 mL) were added N-(piperidin-4-ylmethyl)sulfamide hydrochloride (0.12 g, 0.49 mmol), 0.1M K₂CO₃ in H₂O (12.2 mL, 1.22 mmol). Reaction mixture was stirred at 90° C. for 16 h and the reaction was monitored by TLC. The reaction mixture was diluted with ethyl acetate (20 mL), washed with water (2 x 10 mL) followed by brine (10 mL), dried over anhydrous sodium sulphate, filtered and evaporated under reduced pressure. The crude residue was purified by combiflash using 70% ethyl acetate in hexane to afford N-((1-(6-(3,4-dimethoxyphenyl)pyrimidin-4-yl)piperidin-4-yl)methyl)sulfamide as off-white solid (0.045 g, 22% over 2 steps). LC-MS (ES) m/z = 408.16 [M+H]⁺. ¹H NMR (400 MHz, DMSO-d₆): δ 8.49 (s, 1 H), 7.74 (d, J= 8.4 Hz, 1 H), 7.70 (s, 1 H), 7.21 (s, 1 H), 7.02 (d, J= 8.4 Hz, 1 H), 6.47 - 6.42 (m, 1 H), 6.43 (m, 2 H), 4.53 (d, J= 11.6 Hz, 2 H), 3.83 (s, 3 H), 3.80 (s, 3 H), 2.88 (t, J= 12.4 Hz, 2 H), 2.80 - 2.75 (m, 2 H),1.80 - 1.77 (m, 3 H), 1.09 - 1.07 (m, 2 H); HPLC purity 99.33%.

Example 38: Synthesis of N-(2-(1-(6-(3,4-dimethoxyphenyl)pyrimidin-4-yl)piperidin-4-yl)ethyl)sulfamide

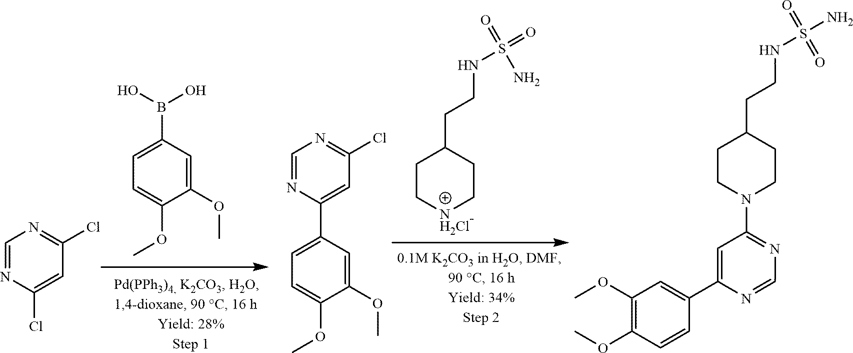

### Step 1 Synthesis of 4-Chloro-6-(3,4-Dimethoxyphenyl)Pyrimidine

Procedure: To a stirred solution of 4,6-dichloropyrimidine (1.0 g, 6.71 mmol) in 1,4-dioxane (10 mL) were added (3,4-dimethoxyphenyl) boronic acid (0.85 g, 4.69 mmol), potassium carbonate (1.25 g, 9.06 mmol, in 14 mL H₂O) resulting mixture was degassed for 15 mins with argon and added Pd(PPh₃)₄ (0.19 g, 0.16 mmol) degassed for another 10 mins. Resulting mixture was stirred for 16h at 90° C. Progress of the reaction was monitored by TLC. Reaction mixture was filtered through celite bed and diluted with ethyl acetate (30 mL), washed with water (2 x 10mL) followed by brine (10mL), dried over anhydrous sodium sulphate, filtered and evaporated under reduced pressure. The crude residue was purified by combiflash using 18% ethyl acetate in hexane to afford 4-chloro-6-(3,4-dimethoxyphenyl)pyrimidine as off-white solid (0.47 g, 28%). LC-MS (ES) m/z = 251.1 [M+H]⁺

### Step 2: Synthesis of N-(2-(1-(6-(3,4-Dimethoxyphenyl)Pyrimidin-4-yl)Piperidin-4-yl) Ethyl) Sulfamide:

Procedure: To a stirred solution of 4-chloro-6-(3,4-dimethoxyphenyl)pyrimidine (0.1 g, 0.4 mmol) in DMF (3 mL) were added N-(2-(piperidin-4-yl)ethyl)sulfamide hydrochloride (0.12 g, 0.44 mmol), 0.1M K₂CO₃ in H₂O (6.0 mL, 0.6 mmol). The reaction mixture was stirred at 90° C. for 16 h. The progress of the reaction was monitored by TLC. The reaction mixture was diluted with ethyl acetate (30 mL), washed with water (2 x 10 mL) followed by brine (10 mL), dried over anhydrous sodium sulphate, filtered and evaporated under reduced pressure. The crude residue was purified by combiflash using 60-70% ethyl acetate in hexane to afford N-(2-(1-(6-(3,4-dimethoxyphenyl)pyrimidin-4-yl)pipen*din-4-yl)ethyl)sulfarnide as white solid (0.055 g, 34%). ¹H NMR (400 MHz, DMSO-d₆): δ 8.48 (s, 1 H), 7.74 (d, J= 8.4 Hz, 1 H), 7.70 (s, 1 H), 7.20 (s, 1 H), 7.02 (d, J= 8.8 Hz,), 6.43 (s, 2 H), 6.40 (t, J = 6.0 Hz, 1 H), 4.43 (d, J= 12.4 Hz, 2H), 3.83 (s, 3H), 3.80 (s, 3 H), 2.94 - 2.85 (m, 4 H), 1.75 - 1.71 (m, 3 H), 1.40 - 1.42 (m, 2H), 1.08 - 1.05 (m, 2H). LC-MS (ES) m/z = 422.2 [M+H]⁺; HPLC purity: 98.78%.

Example 39: Synthesis of N-(1-(5,6-dimethylpyrimidin-4-yl)piperidin-4-yl)sulfamide

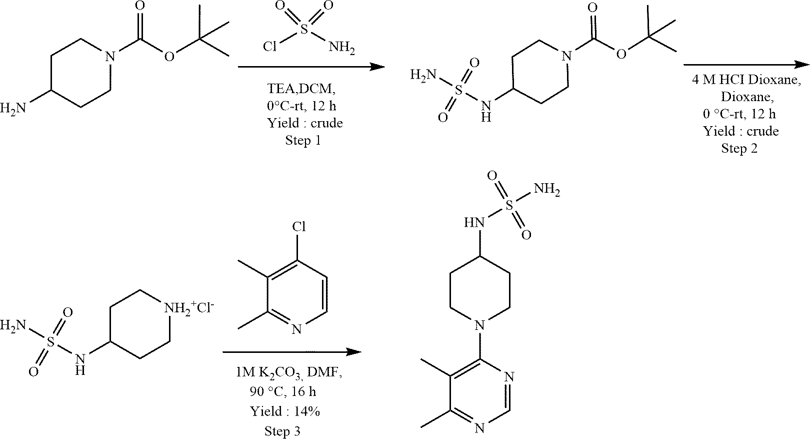

### Step 1: Synthesis of Tert-Butyl 4-(Sulfamoylamino)Piperidine-1-Carboxylate

Procedure: To a stirred solution of tert-butyl 4-aminopiperidine-1-carboxylate (0.5 g, 2.50 mmol) in dichloromethane (15 mL) was added triethyl amine (1.05 mL, 7.52 mmol) followed by sulfamoyl chloride (0.57 g, 5.01 mmol) at 0° C. and allowed the reaction mixture to stir at room temperature for 12 h. The progress of the reaction was monitored by TLC. Then the reaction was diluted with water (100 mL) and extracted with dichloromethane (2 × 50 mL), combined organic layers were washed with brine (50 mL). The organic layer was dried over sodium sulphate, filtered and evaporated under reduced pressure to obtain tert-butyl 4-(sulfamoylamino)piperidine-1-carboxylate (0.4 g, crude) as brown liquid. The crude compound was carried out for next step without purification. LC-MS (ES) m/z = 180.1 [M+H]⁺(deboc).

### Step 2: Synthesis of 4-(Sulfamoylamino)Piperidin-1-Ium Chloride

Procedure: To a stirred solution of tert-butyl 4-(sulfamoylamino)piperidine-1-carboxylate(0.4 g, 1.43 mmol) in dioxane(10 mL)and was added 4 M HCl in dioxane(1 mL) at 0° C. and allowed the reaction mixture to stir at room temperature for 12 h. Progress of the reaction was monitored by TLC. Evaporated the solvent under reduced pressure to obtain 4-(sulfamoylamino)piperidin-1-ium chloride (0.35 g crude). The crude residue was carried out for next step without purification. LCMS (ES) m/z = 180.1 [M+H]⁺.

### Step 3: Synthesis of N-(1-(5,6-Dimethylpyrimidin-4-yl)Piperidin-4-yl)Methane Sulfonamide

Procedure: To a stirred solution of 4-(sulfamoylamino)piperidin-1-ium chloride (0.10 g, 0.49 mmol) and 4-chloro-5,6-dimethylpyrimidine (0.06 g, 0.41 mmol) in dimethyl formamide (4 mL)was added potassium carbonate (0.17 g, 1.24 mmol) and heated the reaction mixture to 90° C. for 16 h. The progress of the reaction was monitored by TLC. The reaction mixture was diluted with water (20 mL), extracted with dichloromethane (2 x 40 mL). The combined organic layer ware washed with brine (20 mL), dried over anhydrous sodium sulphate, filtered and evaporated under reduced pressure to obtain crude compound. The crude residue was purified by prep-HPLC. Column: Intersil ODS 3 V (250 mmx4.6 mmx5 mic), Mobile phase (A): 0.1% TFA in water, Mobile phase(B): ACN, Flow rate: 1.0 mL/min to afford N-(1-(5,6-dimethylpyrimidin-4-yl)piperidin-4-yl)sulfamide (0.03 g, 14%) as white solid. ¹HNMR (400 MHz, DMSO-d₆): 8 8.37 (s, 1 H), 6.59 (d, J= 7.2 Hz, 1 H), 6.46 (s, 2 H), 3.58-3.62 (m, 2 H), 3.27 (s, 1 H), 2.84 (t, J= 11.2 Hz, 2 H), 2.30 (s, 3 H), 2.07 (s, 3 H), 1.92-1.95 (m, 2 H), 1.46-1.55 (m, 2 H). LC-MS (ES) m/z = 286.2 [M+H]⁺. HPLC purity: 99.9 %.

Example 40: N-(2-(4-(6,7-dimethoxyquinazolin-4-yl)piperazin-1-yl)ethyl)sulfamide

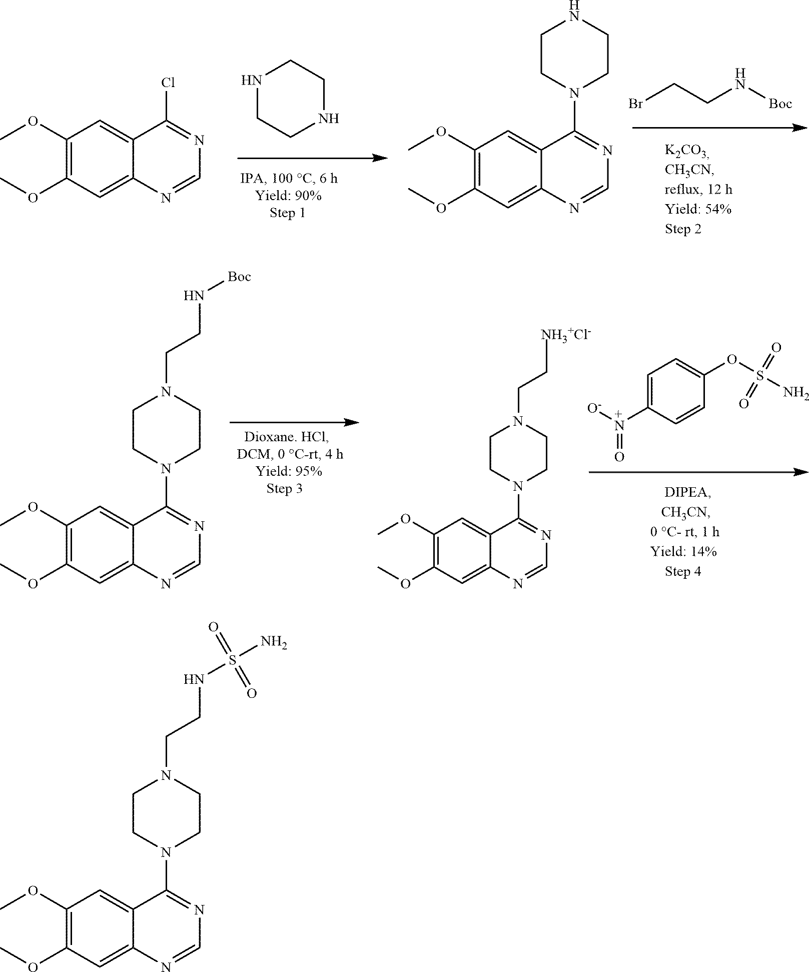

### Step 1: Synthesis of 6,7-Dimethoxy-4-(Piperazin-1-yl)Quinazoline

Procedure: To a stirred solution of 4-chloro-6,7-dimethoxyquinazoline (5 g, 22.261 mmol) in isopropyl alcohol (100 mL) was added piperazine (5.883 g, 66.789 mmol) and solution heated to 100° C. for 6 h. The progress of the reaction was monitored by TLC. Reaction mixture was cooled and solid filtered, washed with ether and dried under vacuum to afford 6,7-dimethoxy-4-(piperazin-1-yl)quinazoline as an off-white solid (5.5 g, 90.09 %). LC-MS (ES) m/z = 275.2 [M+H]⁺.

### Step 2: Synthesis of Tert-Butyl (2-(4-(6,7-Dimethoxyquinazolin-4-yl)Piperazin-1-yl)Ethyl)Carbamate

Procedure: To a stirred solution of 6,7-dimethoxy-4-(piperazin-1-yl)quinazoline (0.3 g, 1.097 mmol) and potassium carbonate (0.379 g, 2.743 mmol) in acetonitrile (9 mL) was added tert-butyl (2-bromoethyl)carbamate (0.368 g, 1.646 mmol) and the mixture was heated to reflux for 12 h. The progress of the reaction was monitored by TLC. Solvent was concentrated and residue diluted with water. Organic compound was extracted with ethyl acetate (3 x 30 mL), dried over sodium sulphate, concentrated. Crude was purified by gradient column chromatography using methanol in dichloromethane to afford tert-butyl (2-(4-(6,7-dimethoxyquinazolin-4-yl)piperazin-1-yl)ethyl)carbamate as colorless liquid (0.250 g, 54.58 %). LC-MS (ES) m/z = 418.1 [M+H]⁺.

### Step 3: Synthesis of 2-(4-(6,7-Dimethoxyquinazolin-4-yl)Piperazin-1-yl)Ethan-1-Aminium Chloride

Procedure: To a stirred solution of tert-butyl (2-(4-(6,7-dimethoxyquinazolin-4-yl)piperazin-1-yl)ethyl)carbamate (0.250 g, 0.598 mmol) in dichloromethane (10 mL) was added dioxane. HC1 (1.5 mL, 4 M solution) at 0° C. and the mixture was stirred at room temperature for 4 h. The progress of the reaction was monitored by TLC. Reaction mixture was filtered and solid washed with dichloromethane, dried under vacuum to afford 2-(4-(6,7-dimethoxyquinazolin-4-yl)piperazin-1-yl)ethan-1-aminium chloride as yellow solid (0.2 g, 95%). LC-MS (ES) m/z = 318.0 [M+H]⁺.

### Step 4: Synthesis of N-(2-(4-(6,7-Dimethoxyquinazolin-4-yl)Piperazin-1-yl)Ethyl)Sulfamide

Procedure: To a stirred solution of 2-(4-(6,7-dimethoxyquinazolin-4-yl)piperazin-1-yl)ethan-1-aminium chloride (0.1 g, 0.282 mmol) and 4-nitrophenyl sulfamate (0.073 g, 0.339 mmol) in acetonitrile (3 mL) was added diisopropyl ethylamine (0.14 mL, 0.843 mmol) at 0° C. and the mixture was stirred at room temperature for 1 h. The progress of the reaction was monitored by TLC. Solvent was concentrated and residue diluted with water. Organic compound was extracted with ethyl acetate (3 x 20 mL), dried over sodium sulphate, concentrated. Crude was purified by gradient column chromatography using methanol in dichloromethane to afford N-(2-(4-(6,7-dimethoxyquinazolin-4-yl)piperazin-1-yl)ethyl)sulfamide as off-white solid (14.28 %). ¹HNMR (400 MHz, DMSO-d₆): 8 8.52 (s, 1 H), 7.20 (s, 1 H), 7.11 (s, 1 H), 6.53 (bs, 2 H), 6.25 (t, J = 6 Hz, 1 H), 3.91 (s, 3 H), 3.89 (s, 3 H), 3.59 (m, 4 H), 3.06 -3.01 (m, 2 H), 2.61 (m, 4 H), 2.52 (m, 2 H). LC-MS (ES) m/z = 397.3 [M+H]⁺. HPLC purity of 98.99 %.

Example 41: Synthesis of 6,7-dimethoxy-4-(5-((4-methylpiperazin-1-yl)sulfonyl)-3,4-dihydroisoquinolin-2(1H)-yl)quinazoline

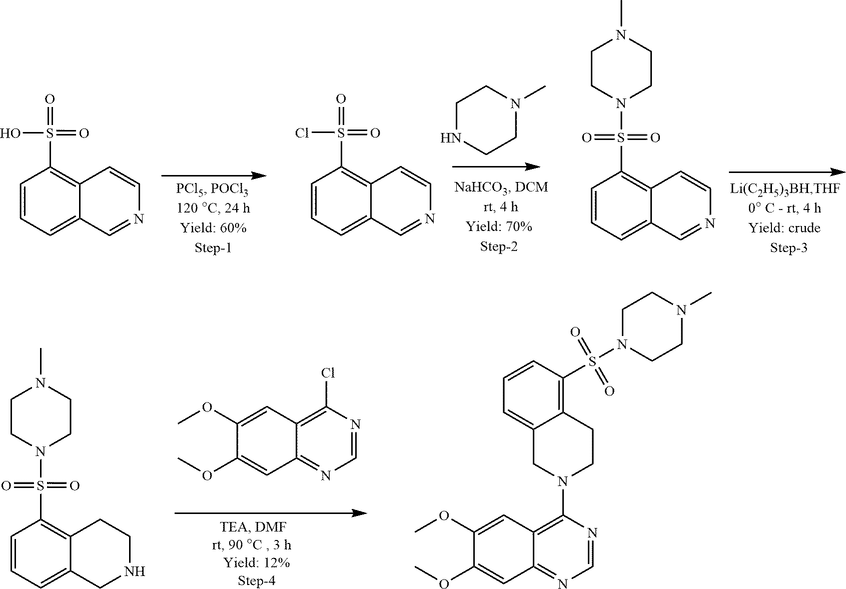

### Step 1 Synthesis of Isoquinoline-5-Sulfonyl Chloride

Procedure: To a stirred solution of isoquinoline-5-sulfonic acid (2.0 g, 9.569 mmol) in POCl₃(30 mL) was added PCl₅ (2.85 mL, 28.708 mmol), at 0° C. Reaction mixture was stirred for 24 h at 120° C. Progress of the reaction was monitored by TLC. Reaction mixture was cooled RT and diluted with DCM (100 mL), Then stirred for 30 minutes, solid was precipitated, filtered the solid and dried under vacuum completely to afford isoquinoline-5-sulfonyl chloride as off white solid, (1.3 g, Y:60%). LC-MS m/z calcd for [M+H]⁺228.1.

### Step 2 Synthesis of 5-((4-Methylpiperazin-1-yl)Sulfonyl)Isoquinoline

Procedure: To a stirred solution ofisoquinoline-5-sulfonyl chloride (0.500 g, 2.202 mmol) in DCM (10 mL), was added 1-methylpiperazine (0.29 mL, 2.643 mmol), and NaHCO₃(0.370 g, 4.405 mmol) at RT. Reaction mixture was stirred at RT for 4 h. reaction was monitored by TLC. The reaction mixture was diluted with water and extracted with DCM (2 x 10 mL). The combined organic layer was washed with brine (10 mL), dried over anhydrous sodium sulphate, filtered and evaporated under reduced pressure. The crude residue was purified by combiflash using methanol in dichloromethane to afford 5-((4-methylpiperazin-1-yl)sulfonyl)isoquinoline as off white solid (0.450 g, Y:70%).LC-MS m/z calcd for [M+H]⁺ 292.2.

### Step 3 Synthesis of 5-((4-Methylpiperazin-1-yl)Sulfonyl)-1,2,3,4-Tetrahydroisoquinoline

Procedure : To a stirred solution of 5-((4-methylpiperazin-1-yl)sulfonyl)isoquinoline (0.3 g, 1.030 mmol) in THF (10 mL) was added Super hydride (2.5 mL, 2.577 mmol) at 0° C. Reaction mixture was stirred for 4 h at RT. Progress of the reaction was monitored by TLC. Reaction mixture was diluted with water (10 mL), extracted with ethyl acetate (2 x 10 mL). The combined organic layer was washed with brine (10 mL), dried over anhydrous sodium sulphate, filtered and evaporated under reduced pressure. The crude residue was purified by combiflash using methanol in dichloromethaneto afford 5-((4-methylpiperazin-1-yl)sulfonyl)-1,2,3,4-tetrahydroisoquinoline colorless liquid, (0.250 g, Crude). LC-MS m/z calcd for [M+H]⁺296.2.

### Step 4: Synthesis of 6,7-Dimethoxy-4-(5-((4-Methylpiperazin-1-yl)Sulfonyl)-3,4-Dihydroisoquinolin-2(1H)-yl)Quinazoline:

Procedure: To a stirred solution of 5-((4-methylpiperazin-1-yl)sulfonyl)-1,2,3,4-tetrahydroisoquinoline (0.200 g, 0.677 mmol) in DMF(5 mL), was added4-chloro-6,7-dimethoxyquinazoline (0.152 g, 0.677 mmol), and TEA (0.27 mL, 2.003 mmol) at RT. Reaction mixture was stirred at 80° C. for 4 h. Reaction was monitored by TLC. The reaction mixture was diluted with water and extracted with ethyl acetate (2 x 10 mL). The combined organic layer was washed with brine (10 mL), dried over anhydrous sodium sulphate, filtered and evaporated under reduced pressure. The crude residue was purified by combiflash using methanol in dichloromethane to afford 6,7-dimethoxy-4-(5-((4-methylpiperazin-1-yl)sulfonyl)-3,4-dihydroisoquinolin-2(1H)-yl)quinazoline as off white solid (0.060 g, Y: 12%).′H NMR (400 MHz, DMSO-d₆ :δ 8.54 (s, 1 H), 7.75 (d, J =7.6 Hz, 1 H), 7.63(s, 1 H), 7.45 (s, 1 H), 7.27 (s, 1 H), 7.23(s, 1 H), 4.92 (s, 2 H), 3.95 (d, J = 7.2 Hz, 7 H), 3.41 (s, 2 H), 3.083 (s, 4 H), 2.337 (s, 4 H), 2.158 (s, 4 H). LC-MS (ES) m/z 484.0 [M+H]⁺ , HPLC purity 99.8 %.

Example 42: 2-(1-(6,7-dimethoxyquinazolin-4-yl)piperidin-4-yl)ethan-1-amino(N-ethyl-N-methyl)sulfonamide

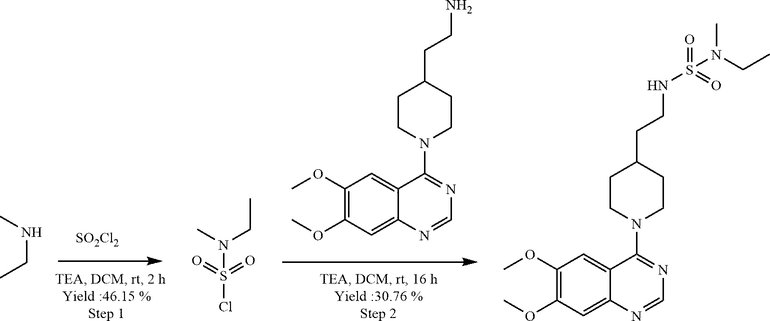

### Step 1: Synthesis of Ethyl(Methyl)Sulfamoyl Chloride

Procedure: To a stirred solution of N-methylethanamine (0.5 g, 8.474 mmol) in DCM (20 mL) was added TEA (1.28 g, 12.67 mmol) followed by sulfuryl dichloride (1.13 g, 8.474 mmol). Reaction mixture was stirred for 2 h at room temperature. Progress of the reaction was monitored by TLC. Reaction mixture was diluted with water (20 mL), extracted with ethyl acetate (2 x 50 mL). The combined organic layer was washed with brine (10 mL), dried over anhydrous sodium sulphate, filtered and evaporated under reduced pressure. The crude residue was washed with diethylether to afford ethyl(methyl)sulfamoyl chloride as a liquid, (0.6 g, 46.15 %).

### Step 2: Synthesis of 2-(1-(6,7-Dimethoxyquinazolin-4-yl)Piperidin-4-yl)Ethan-1-Amino(N-Ethyl-N-Methyl)Sulfonamide

Procedure: To a stirred solution of 2-(1-(6,7-dimethoxyquinazolin-4-yl)piperidin-4-yl)ethan-1-amine (0.1 g, 0.3164 mmol) in dichloromethane (10 mL) was added triethylamine (0.1 g, 099 mmol) followed by ethyl(methyl)sulfamoyl chloride (0.049 g, 0.3164 mmol). Reaction mixture was stirred at room temperature for 16 h, reaction was monitored by TLC. The reaction mixture was diluted with water (50mL), extracted with dichloromethane (2 x 50 mL). The combined organic layer was washed with brine (10mL), dried over anhydrous sodium sulphate, filtered and evaporated under reduced pressure. The crude residue was purified by combiflash using methanol in dichloromethane to afford 2-(1-(6,7-dimethoxyquinazolin-4-yl)piperidin-4-yl)ethan-l-amino(N-ethyl-N-methyl)sulfonamide as white solid (0.04 g, 30.76 %). ¹HNMR (400 MHz, DMSO-d₆): 8 8.48 (s, 1 H),7.17 (s, 1 H), 7.08 (s, 1 H), 7.03 (t, J= 5.6 Hz, 1 H), 4.11 (d, J= 12.8 Hz, 2 H), 3.88-3.90 (m, 6 H), 3.02-3.10 (m, 2 H),2.99 (t, J= 12 Hz, 2 H),2.87-2.92 (m, 2 H),2.65(s, 3 H),1.75 (d, J= 12 Hz, 2 H), 1.66 (s, 1 H), 1.41-1.47 (m, 2 H), 1.29-1.32 (m, 2 H), 1.07 (t, J= 6.8 Hz, 3 H). HPLC purity 99.34 %. LC-MS (ES) m/z = 438.2 [M+H]⁺.

Example 43: Synthesis of N-(2-(1-(6,7-dimethoxyquinolin-4-yl)piperidin-4-yl)ethyl)sulfamide

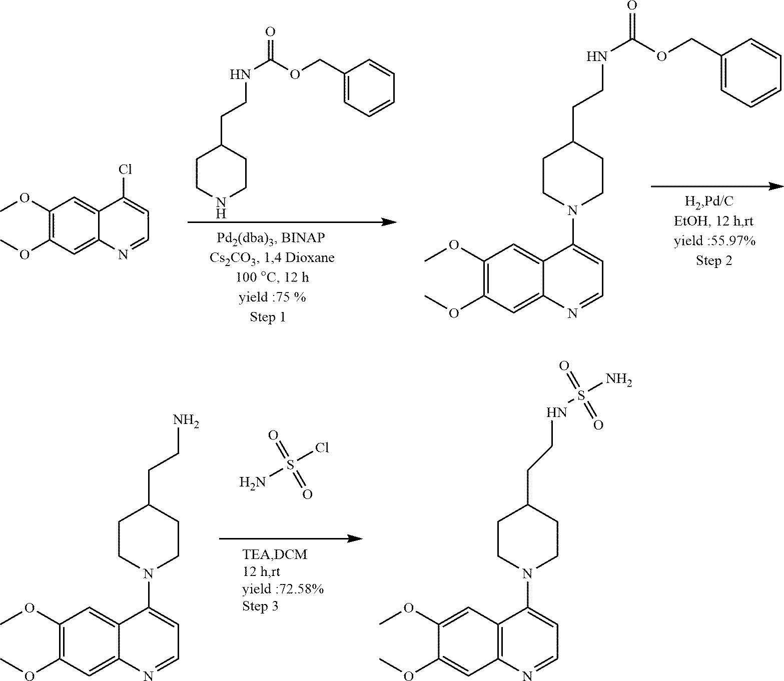

### Step 1: Synthesis of Benzyl (2-(1-(6,7-Dimethoxyquinolin-4-yl)Piperidin-4-yl)Ethyl)Carbamate

Procedure: To a stirred solution of 4-chloro-6,7-dimethoxyquinoline (0.3 g, 1.34 mmol) in dioxane, was added benzyl (2-(piperidin-4-yl)ethyl)carbamate hydrochloride (0.389 g, 1.484 mmol), and Cs₂CO₃(1.3 g, 3.99 mmol). Reaction mixture was stirred for 10 mins degassed. Finally were added Pd₂(dba)₃(0.123 g, 0.1343 mmol) and BINAP (0.167 g, 0.268 mmol).Reaction mixture was stirred for 12 h at 100° C. in seal tube . Progress of the reaction was monitored by TLC. Reaction mixture was diluted with water (20 mL), extracted with ethyl acetate (2 x 50 mL). The combined organic layer was washed with brine (10 mL), dried over anhydrous sodium sulphate, filtered and evaporated under reduced pressure. The crude residue was purified by combiflash using methanol in dichloromethane to afford benzyl (2-(1-(6,7-dimethoxyquinolin-4-yl)piperidin-4-yl)ethyl)carbamate as white solid, (0.45 g, 75 %). ¹HNMR (400 MHz, DMSO-d₆): δ 8.45 (d, J= 4.8 Hz, 1 H),7.23-7.33 (m, 7 H), 7.14 (s, 1 H), 6.75-6.89 (m, 1 H), 5.00(s, 2 H), 3.88 (d, J= 2.4 Hz, 6 H), 3.44 (d, J= 11.2 Hz, 2 H), 3.08 (d, J= 6.0 Hz, 2 H), 2.69 (t, J= 11.2 Hz, 2 H), 1.82(d, J= 10.4 Hz, 2 H), 1.46 (t, J = 4.0 Hz, 5 H).LC-MS (ES) m/z = 450.2[M+H]⁺.

### Step 2: Synthesis of 2-(1-(6,7-Dimethoxyquinolin-4-yl)Piperidin-4-yl)Ethan-1-Amine

Procedure: To a solution of benzyl (2-(1-(6,7-dimethoxyquinolin-4-yl)piperidin-4-yl)ethyl)carbamate (0.4 g, 0.8908 mmol) in ethanol (20 ml) was added Pd/C (10%) under hydrogen atmosphere, then the reaction mixture was stirred at room temperature for 12 h. The reaction mixture was filtered through celite, washed with ethyl acetate, the filtrate was concentrated under reduced pressure to give crude residue which was purified by column chromatography over silica gel using 10% methanol /dichloromethane as eluent to give the title compound as off-white solid (0.15 g, 55.97 %).¹HNMR (400 MHz, CDC1₃): δ 8.54 (d, J= 4.8 Hz, 1 H), 7.39 (s, 1 H), 7.23 (t, J= 9.6 Hz, 1 H),6.75 (d, J= 5.2 Hz, 1 H),4.20 (s, 6 H),3.54 (d, J= 12.4 Hz, 2 H), 2.85 (s, 1 H), 2.76-2.85 (m, 4 H), 1.90 (d, J= 11.2 Hz, 2 H),1.60-1.64 (m, 5 H),1.41-1.44 (m, 1 H). LC-MS (ES) m/z = 316.2 [M+H]⁺.

### Step 3: Synthesis of N-(2-(1-(6,7-Dimethoxyquinolin-4-yl)Piperidin-4-yl)Ethyl)Sulfamide

Procedure: To a stirred solution of 2-(1-(6,7-dimethoxyquinolin-4-yl)piperidin-4-yl)ethan-1-amine(0.05 g, 0.158 mmol) in dichloromethane (10 mL) was added triethylamine (0.1 mL, 0.396 mmol) followed by sulfamoyl chloride (0.02 g, 0.190 mmol). Reaction mixture was stirred at room temperature for 12 h, reaction was monitored by TLC. The reaction mixture was diluted with water (50 mL), extracted with dichloromethane (2 x 50 mL). The combined organic layer was washed with brine (10 mL), dried over anhydrous sodium sulphate, filtered and evaporated under reduced pressure. The crude residue was purified by combiflash using methanol in dichloromethane to afford N-(2-(1-(6,7-dimethoxyquinolin-4-yl)piperidin-4-yl)ethyl)sulfamide white solid (0.045 g, 72.58 %).¹HNMR (400 MHz, DMSO-d₆): 8 8.46 (d, J= 5.2 Hz, 1 H), 7.28 (s, 1 H), 7.16 (s, 1 H), 6.86 (d, J= 5.2 Hz, 1 H), 6.43 (t, J= 5.6 Hz, 2 H), 3.89 (s, 6 H), 3.55 (d, J= 11.6 Hz, 2 H), 2.93-2.98 (m, 2 H), 2.80 (t, J= 12.0 Hz, 2 H), 1.82-1.89 (m, 3 H), 1.62 (bs, 1 H), 1.43-1.54 (m, 4 H), 1.22(s, 1 H) .HPLC purity 99.54 %. LC-MS (ES) m/z =395.2 [M+H]⁺.

Example 44: Synthesis of N-(4,5-dimethoxy-2-(4-(2-(sulfamoylamino)ethyl)piperidine-1-carbonyl)phenyl)acetamide

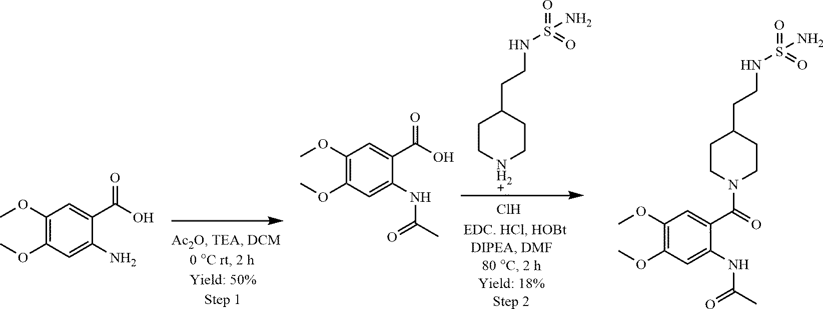

### Step 1: Synthesis of 2-Acetamido-4,5-Dimethoxybenzoic Acid

Procedure: To a stirred solution of 2-amino-4,5-dimethoxybenzoic acid (0.5 g, 2.53 mmol) in dichloromethane (10 mL) was added triethyl amine (0.54 mL, 3.80 mmol) and acetic anhydride (0.28 mL 3.04 mmol) at 0° C. and stirred at room temperature for 2 h. The progress of the reaction was monitored by TLC. Then the reaction was quenched with water (100 mL) and extracted with dichloromethane (2 × 50 mL), combined organic layers were washed with brine (50 mL). The organic layer was dried over sodium sulphate, filtered and evaporated under reduced pressure to obtain crude compound. The crude residue was purified by gradient column chromatography using 3% methanol in dichloromethane to afford 2-acetamido-4,5-dimethoxybenzoic acid (0.30 g, 50%) as Brown solid. ¹HNMR (400 MHz, DMSO-d₆): δ 13.28 (s, 1 H), 11.12 (s, 1 H), 8.24 (s, 1 H), 7.04 (s, 1 H), 3.78-3.72 (m, 6 H), 2.10 (s, 3 H).LC-MS (ES) m/z = 240.1 [M+H]⁺.

### Step 2: Synthesis of N-(4,5-Dimethoxy-2-(4-(2-(Sulfamoylamino)Ethyl)Piperidine-1-Carbonyl)Phenyl)Acetamide

Procedure: To a stirred solution of 2-acetamido-4,5-dimethoxybenzoic acid (0.10 g, 0.41 mmol) in dimethyl formamide (3 mL) was added diisopropyl ethylamine (0.23 mL, 1.25 mmol) and added EDC.HC1 (0.12 g, 0.62 mmol), HOBt (0.084 g, 0.62 mmol) stirred at room temperature for 5 mins and then added N-(2-piperidin-4-ylethyl)sulfamidedihydrochloride (0.12 g, 0.46 mmol) heated into 80° C. for 2 h. The progress of the reaction was monitored by TLC. Then the reaction was quenched with ice cold water (100 mL) and extracted with ethyl acetate (3 × 100 mL), combined organic layers were washed with brine (50 mL). The organic layer was dried over sodium sulphate, filtered and evaporated under reduced pressure to obtain crude compound. The crude residue was purified by gradient column chromatography using 4% methanol in dichloromethane to afford N-(4,5-dimethoxy-2-(4-(2-(sulfamoylamino)ethyl)piperidine-1-carbonyl)phenyl)acetamide(0.030 g, 18%) as White solid. ¹HNMR (400 MHz, DMSO-d₆): δ 9.36 (s, 1 H), 7.12 (s, 1 H), 6.75 (s, 1 H), 6.36-6.41 (m, 3 H), 4.5 (b, 1 H), 3.72 (s, 6 H), 3.4 (b, 1 H), 2.85-2.90 (m, 2 H), 2.65 (b, 1 H), 1.96 (s, 3 H), 1.57-1.60 (m, 3 H), 1.38-1.40 (m, 2 H), 1.04-1.11 (m, 2 H), 0.92-0.93 (m, 1 H).LC-MS (ES) m/z = 429.2 [M+H]⁺. HPLC purity 99.28% at 280 nm.

Example 45: N-(2-(1-(2-(dimethylamino)-4,5-dimethoxybenzoyl)piperidin-4-yl)ethyl)sulfamide

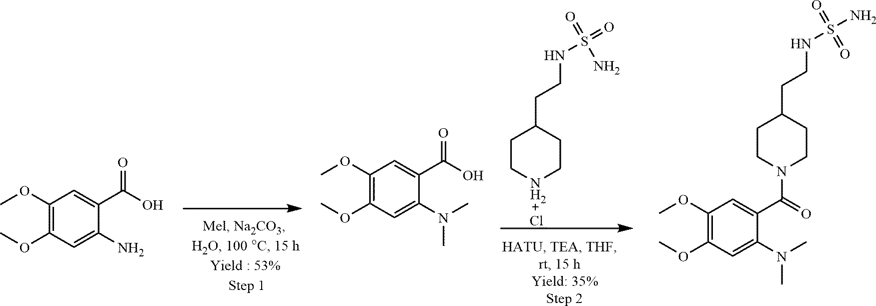

### Step 1: Synthesis of 2-(Dimethylamino)-4,5-Dimethoxybenzoic Acid

Procedure: To a stirred solution of compound 2-amino-4,5-dimethoxybenzoic acid (1 g, 5.071 mmol) in water (3 mL) were added sodium carbonate (0.429 g, 4.057 mmol) and methyl iodide (1.57 mL, 25.356 mmol). The resulting mixture was refluxed for 15 h. The solid formed was filtered and the residue was washed with water and dried under reduced pressure to get title compound as Ash coloured solid (0.6 g, 53%). ¹HNMR (400 MHz, DMSO-d₆): 8 7.46 (s, 2 H), 3.89 (s, 3 H), 3.80 (s, 3 H), 2.99 (s, 6 H).

### Step 2: Synthesis of N-(2-(1-(2-(Dimethylamino)-4,5-Dimethoxybenzoyl)Piperidin-4-yl)Ethyl)Sulfamide

Procedure: To a stirred solution of compound 2-(dimethylamino)-4,5-dimethoxybenzoic acid (0.1 g, 0.443 mmol) in THF (10 mL) were added 4-(2-(sulfamoylamino)ethyl)piperidin-1-ium chloride (0.11 g, 0.488 mmol), Triethylamine (0.31 mL, 2.21 mmol) and HATU (0.25 g, 0.665 mmol). The resulting mixture was stirred at room temperature for 15 h. The progress of the reaction was monitored by TLC. Then the reaction was quenched with water (10 mL) and extracted with ethyl acetate (3 × 50 mL), The combined organic layers were was dried over anhydrous sodium sulphate, filtered and evaporated under reduced pressure, purified by combiflash purifier using 6% methanol in dichloromethane to afford N-(2-(1-(2-(dimethylamino)-4,5-dimethoxybenzoyl)piperidin-4-yl)ethyl)sulfamide (0.06 g, 35 %). ¹HNMR (400 MHz, DMSO-d₆): 8 8.53 (s, 1 H), 7.77 (d, J= 7.6 Hz, 1 H), 7.51 (d, J= 7.2 Hz, 1 H), 7.43 (bs, 2 H), 7.39-7.35 (m, 1 H), 7.23 (d, J= 8.0 Hz, 2 H), 4.89 (s, 2 H), 3.94-3.92 (m, 8 H), 3.45-3.44 (m, 2 H). m/z = 415.2 [M+H]⁺, HPLC = 99.79 at 280 nm..

Example 46: N-(2-(1-(2-amino-4,5-dimethoxybenzoyl)piperidin-4-yl)ethyl)sulfamide

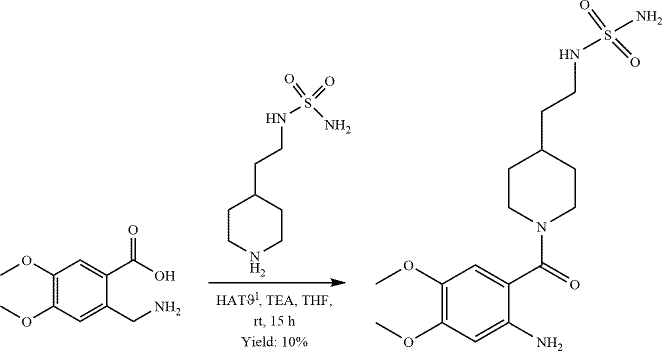

Procedure: To a stirred solution of compound 2-amino-4,5-dimethoxybenzoic acid (0.1 g, 0.507 mmol) in THF (10 mL) were added 4-(2-(sulfamoylamino)ethyl)piperidin-1-ium chloride (0.135 g, 0.557 mmol), Triethylamine (0.35 mL, 2.53 mmol) and HATU (0.289 g, 0.760 mmol). The resulting mixture was stirred at room temperature for 15 h. The progress of the reaction was monitored by TLC. Then the reaction was quenched with water (10 mL) and extracted with ethyl acetate (3 × 50 mL), The combined organic layers were washed with water (5 mL), brine (10 mL) and dried over anhydrous sodium sulphate, filtered and evaporated under reduced pressure, purified by combiflash purifier using 8% methanol in dichloromethane to afford N-(2-(1-(2-amino-4,5-dimethoxybenzoyl)piperidin-4-yl)ethyl)sulfamide (0.02 g , 10%). ¹HNMR (400 MHz, DMSO-d₆): δ 6.55 (s, 1 H), 6.41 (bs, 2 H), 6.38-6.37 (m, 1 H), 6.35 (s, 1 H), 4.86 (s, 2 H), 3.98 (bs, 2 H), 3.68-3.61 (m, 6H), 2.89-2.86 (m, 2H), 2.82-2.78 (m, 2 H), 1.65-1.62 (m, 3 H), 1.40-1.39 (m, 2 H), 1.07-1.04 (m, 2 H). m/z = 387.2 [M+H]⁺, HPLC = 99.34 at 280 nm.

Example 47: N (2-(1-(3,4-dimethoxybenzoyl)piperidin-4-yl)ethyl)sulfamide

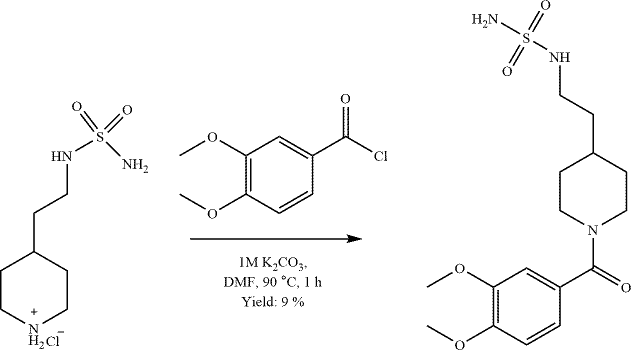

Procedure: To a stirred solution of compound N (2-(piperidin-4-yl)ethyl)sulfamide hydrochloride (0.15 g, 0.615 mmol) in Dimethyl formamide (5 mL) was added 1M K₂CO₃ (1.2 mL, 1.23 mmol) and heated to 90° C. then3,4-dimethoxybenzoyl chloride(0.185 g, 0.923 mmol) was added at 90° C. and stirred for 1.0 h. The progress of the reaction was monitored by TLC. Then the reaction was quenched with water and extracted with ethyl acetate (2 × 50 mL), combined organic layers were washed with water (30 mL) followed by brine (30 mL) and the organic layer was dried over sodium sulphate, filtered and evaporated under reduced pressure.

NOTE:- The crude residue purified by combiflash chromatography using ethyl acetate in n-hexane and the compound was eluted at 6 %ethyl acetate in n-hexane to afford N-(2-(1-(3,4-dimethoxybenzoyl)piperidin-4-yl)ethyl)sulfamide as an off white solid (0.03 g , 8.7 %). ¹HNMR (400 MHz, DMSO-d₆): 8 6.89 - 6.97 (m, 3 H), 6.39 (t, J = 5.6 Hz, 3 H), 3.76 (d, J = 6.8 Hz, 6H), 2.86 -2.91(m, 5 H), 1.63 (s, 3 H), 1.41 (d, J = 6.8 Hz, 2 H), 1.22 (s, 1 H), 1.05 (d, J = 10.4 Hz, 2 H). LC-MS (ES) m/z = 372.3 [M+H]⁺.

Example 48: Synthesis of N-(2-(1-(3-methoxybenzoyl)piperidin-4-yl)ethyl)sulfamide

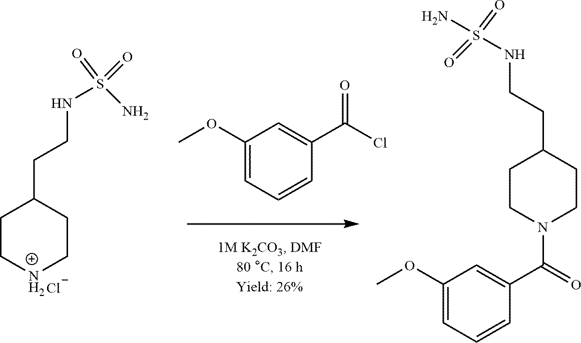

Procedure: To a stirred solution of 4-(2-(sulfamoylamino)ethyl)piperidin-1-ium (0.1 g, 0.410 mmol) in N,N-dimethylformamide (4 mL) was added 1 M potassium carbonate (0.82 mL, 0.82 mmol) and 3-methoxybenzoyl chloride (0.104 g, 0.615 mmol). The resulting mixture was stirred at 80° C. for 16.0 h. The progress of the reaction was monitored by TLC (5 % Methanol in dichloromethane). Then the reaction mixture distilled under vacuum. The crude residue was purified by gradient column chromatography using Methanol in dichloromethane to afford N-(2-(1-(3-methoxybenzoyl)piperidin-4-yl)ethyl)sulfamide as an off-white solid (0.037 g , 26 %). ¹HNMR (400 MHz, DMSO-d₆): 8 7.32 (t, J= 8 Hz, 1 H), 6.99 - 6.96 (m, 1 H), 6.89 - 6.86 (m, 2 H), 6.42 - 6.37 (m, 3 H), 4.43 (bs, 1 H), 3.75 (s, 3 H), 3.49 (bs, 1 H), 2.94 (bs, 1 H), 2.91 - 2.86 (m, 2 H), 2.71(bs, 1 H), 1.69 - 1.6 (m, 3 H), 1.43 - 1.38 (m, 2 H), 1.05 (bs, 2 H).LC-MS (ES) m/z = 342.1 [M+H]⁺.

Example 49: Synthesis of N-(2-(1-(4-methoxybenzoyl)piperidin-4-yl)ethyl)sulfamide

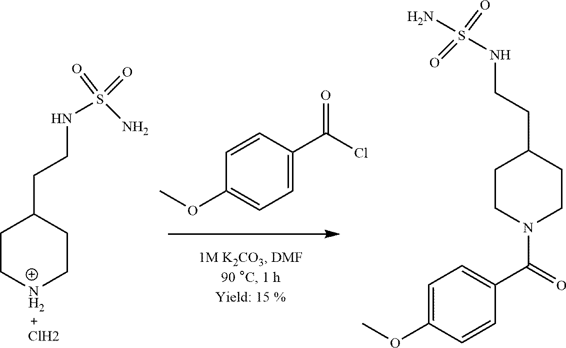

Procedure: To a stirred solution of 2-(piperidin-4-yl)ethan-l-amine (0.1 g, 0.410 mmol) in DMF (5 mL) was added 1 M K₂CO₃ solution (1.0 mL), and 4-methoxybenzoyl chloride(0.08 mL, 0.615 mmol), 0.615 mmol. Reaction mixture was stirred for 1h at 90° C. Progress of the reaction was monitored by TLC. Reaction mixture diluted with water (5 mL), extracted with ethyl acetate (2 x 10 mL). The combined organic layer was washed with brine (7 mL), dried over anhydrous sodium sulphate, filtered and evaporated under reduced pressure. The crude residue was purified by combiflash using methanol in dichloromethane to afford N-(2-(1-(4-methoxybenzoyl)piperidin-4-yl)ethyl)sulfamide as off-white solid, (0.021 g, Yield:15 %). 1HNMR (400 MHz, DMSO-d₆ :δ 7.31 (d, J= 7.2 Hz, 2 H), 6.95 (d, 2 H), 6.42-6.37 (m, 3 H), 4.43 (s, 1 H), 3.72(s, 3 H), 2.91-2.86 (m, 3 H), 1.62 (s, 3 H), 1.43-1.38 (m, 2 H), 1.22 (s, 1 H), 1.12-1.0(s, 2 H).LC-MS (ES) m/z = 342.1[M+H]⁺, HPLC purity 98.07%.

Example 50: Synthesis of N-(2-(1-(benzoyl)piperidin-4-yl)ethyl)sulfamide

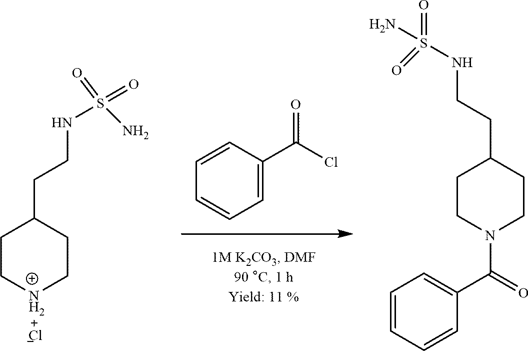

Procedure: To a stirred solution of 2-(piperidin-4-yl)ethan-l-amine (0.1 g, 0.410 mmol) in DMF (5 mL) was added 1M K₂CO₃solution (1.0 mL), and benzoyl chloride (0.086 g, 0.615 mmol). Reaction mixture was stirred for 1 h at 90° C. Progress of the reaction was monitored by TLC. Reaction mixture diluted with water (5 mL), extracted with ethyl acetate (2 x 10 mL). The combined organic layer was washed with brine (7 mL), dried over anhydrous sodium sulphate, filtered and evaporated under reduced pressure. The crude residue was purified by combiflash using methanol in dichloromethaneto afford N-(2-(1-(benzoyl)piperidin-4-yl)ethyl)sulfamide as off white solid, (0.014 g, Yield: 11%). ¹H NMR (400 MHz, DMSO-d₆ :δ 7.41 (d, J= 3.6 Hz, 3 H), 7.33 (d, J =3.6 Hz, 2 H), 6.39(t, J =12, 3 H), 4.43 (s, 1 H), 3.49 (s, 1 H), 2.91-2.86 (m, 3 H), 2.71 (s, 1 H), 1.62(s, 3 H), 1.41(t, J =7.2, 3 H), 1.06 (s, 2 H).LC-MS (ES) m/z= 312.1 [M+H]⁺ , HPLC purity 99.5 %.

Example 51: N-(1-(6,7-dimethoxyquinazolin-4-yl)piperidin-4-yl)sulfamide

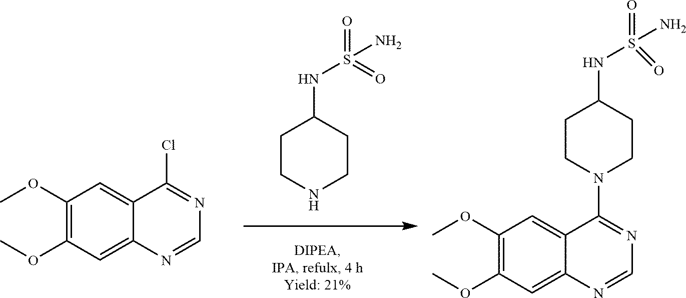

Procedure: To a stirred solution of 4-chloro-6,7-dimethoxyquinazoline (0.1 g, 0.44 mmol) and N-(piperidin-4-yl)sulfamide hydrochloride (0.124 g, 0.578 mmol) in isopropyl alcohol (4 mL) was added diisopropyl ethylamine (0.38 mL, 2.20 mmol) and solution heated to reflux for 4 h. The progress of the reaction was monitored by TLC. Solvent was concentrated and residue was diluted with water. Organic compound extracted using 5% methanol/ dichloromethane solvent (3 x 20 mL), dried over sodium sulphate and concentrated. The crude product was purified by gradient column chromatography using methanol in ethyl acetate to afford N-(1-(6,7-dimethoxyquinazolin-4-yl)piperidin-4-yl)sulfamide as an off white solid (0.035 g, 21%). ¹HNMR (400 MHz, DMSO-d₆): 8 8.50 (s, 1 H), 7.19 (s, 1 H), 7.08 (s, 1 H), 6.64 (d, J = 7.2 Hz, 1 H), 6.51 (bs, 2 H), 4.08 -4.05 (m, 2 H), 3.91 (s, 3 H), 3.89 (s, 3 H), 3.38 - 3.32 (m, 1 H), 3.18 - 3.12 (m, 2 H), 2.04 - 2.01 (m, 2 H), 1.64 -1.61 (m, 2 H). LC-MS (ES) m/z = 368.1 [M+H]⁺ HPLC purity 99.5 %.

Example 52: N-(2-(4-(6,7-dimethoxyquinazolin-4-yl)piperazin-1-yl)-2-oxoethyl)sulfamide

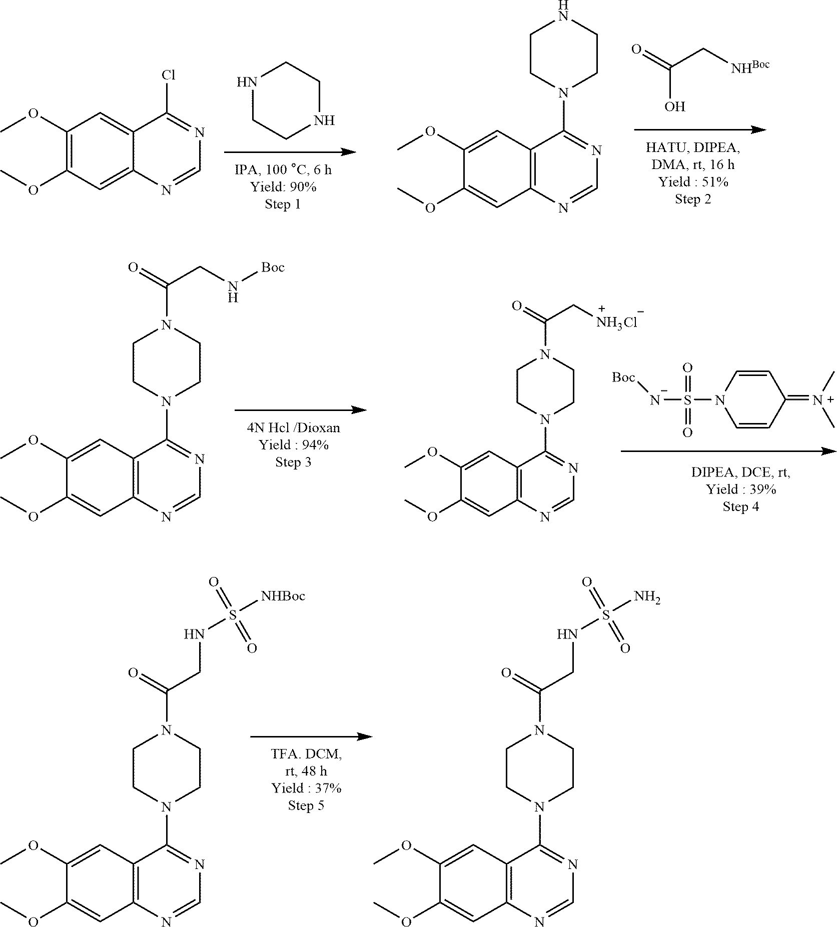

### Step 1: Synthesis of 6,7-Dimethoxy-4-(Piperazin-1-yl)Quinazoline

Procedure: To a stirred solution of 4-chloro-6,7-dimethoxyquinazoline (5 g, 22.261 mmol) in isopropyl alcohol (100 mL) was added piperazine (5.883 g, 66.789 mmol) and solution heated to 100° C. for 6 h. The progress of the reaction was monitored by TLC. Reaction mixture was cooled and solid filtered, washed with ether and dried under vacuum to afford 6,7-dimethoxy-4-(piperazin-1-yl)quinazoline as an off-white solid (5.5 g, 90.1 %). LC-MS (ES) m/z =275.2 [M+H]⁺.

### Step 2: Synthesis of Tert-Butyl (2-(4-(6,7-Dimethoxyquinazolin-4-yl)Piperazin-1-yl)-2-Oxoethyl)Carbamate

Procedure: To a stirred solution of 6,7-dimethoxy-4-(piperazin-1-yl)quinazoline (1 g, 3.64 mmol) in DMA (10 mL) was added (tert-butoxycarbonyl)glycine (0.766 g, 4.37 mmol), DIPEA (1.9 mL,10.92 mmol) and HATU (1.66 g, 4.37 mmol). The resulting mixture was stirred at room temperature for 16 h. The progress of the reaction was monitored by TLC. Then the reaction was quenched with water (10 mL) and extracted with ethyl acetate (3 × 50 mL), The combined organic layers were was dried over anhydrous sodium sulphate, filtered and evaporated under reduced pressure, purified by combiflash using 1-10% methanol in dichloromethane to afford tert-butyl (2-(4-(6,7-dimethoxyquinazolin-4-yl)piperazin-1-yl)-2-oxoethyl)carbamate as an off- white solid (0.8 g , 51%). LC-MS (ES) m/z = 432.2 [M+H]⁺.

### Step 3: Synthesis of 2-Amino-1-(4-(6,7-Dimethoxyquinazolin-4-yl)Piperazin-1-yl)Ethan-1-One Hydrochloride

Procedure: To a stirred solution of tert-butyl (2-(4-(6,7-dimethoxyquinazolin-4-yl)piperazin-1-yl)-2-oxoethyl)carbamate (0.25 g, 0.579 mmol) in dioxane (5 mL) was added dioxane.HCl (20 mL, 4N solution) at 0° C. and solution stirred at room temperature for 2 h. The progress of the reaction was monitored by TLC. Reaction mixture was dried under vacuum. The crude was washed with ethyl acetate and diethyl ether and then dried under vacuum to afford 2-amino-1-(4-(6,7-dimethoxyquinazolin-4-yl)piperazin-1-yl)ethan-1-one hydrochloride as an off-white solid (0.2 g, 94%). ¹HNMR (400 MHz, DMSO-d₆ D2O): 8 8.63 (s, 1 H), 7.29 (s, 1 H), 7.19 (s, 1 H), 4.01 (m, 4 H), 3.93 (s, 3 H), 3.9 (s, 3 H), 3.84 (bs, 2 H), 3.82 (bs, 1 H), 3.63 (m, 3 H); LC-MS (ES) m/z = 332.2 [M+H]⁺; HPLC purity 99.59 %.

### Step 4: Synthesis of Tert-Butyl (N-(2-(4-(6,7-Dimethoxyquinazolin-4-yl)Piperazin-1-yl)-2-Oxoethyl)Sulfamoyl)Carbamate

Procedure: To a stirred solution of 2-(4-(6,7-dimethoxyquinazolin-4-yl)piperazin-1-yl)-2-oxoethan-1-aminium chloride (0.1 g, 0.27 mmol) in dichloroethane (20 mL) was added (tert-butoxycarbonyl)((4-(dimethyliminio)pyridin-l(4H)-yl)sulfonyl)amide (0.089 g, 0.297 mmol) and diisopropylethylenediamine (0.142 mL, 0.81 mmol). Reaction mixture was stirred at room temperature for 48 h, reaction was monitored by TLC. The reaction mixture was diluted with dichloromethane (50 mL), washed with water (2 x 30 mL), dried over anhydrous sodium sulphate, filtered and evaporated under reduced pressure. The crude residue 0.2 g was purified by combiflash using 1-10% methanol in DCM to afford tert-butyl (N-(2-(4-(6,7-dimethoxyquinazolin-4-yl)piperazin-1-yl)-2-oxoethyl)sulfamoyl) carbamate as off-white solid (0.11 g, 39%). LC-MS (ES) m/z = 510.57 [M]⁺.

### Step 5: Synthesis of N-(2-(4-(6,7-Dimethoxyquinazolin-4-yl)Piperazin-1-yl)-2-Oxoethyl)Sulfamide

Procedure: To a stirred solution of tert-butyl (N-(2-(4-(6,7-dimethoxyquinazolin-4-yl)piperazin-1-yl)-2-oxoethyl)sulfamoyl)carbamate (0.08 g, 0.156 mmol) in dry DCM (10 mL) was added TFA (0.12 mL, 1.56 mmol). Reaction mixture was stirred at room temperature for 48 h, reaction was monitored by TLC. The reaction mixture was evaporated under reduced pressure and crude was dissolved in DCM. The organic layer was washed with saturated NaHCO₃ solution, brine solution and dried over anhydrous sodium sulphate. The organic layer was evaporated under reduced pressure. The crude residue was purified by combiflash using 1-15% methanol in DCM to afford N-(2-(4-(6,7-dimethoxyquinazolin-4-yl)piperazin-1-yl)-2-oxoethyl)sulfamide as off-white solid (0.03 g, 37%). ¹HNMR (400 MHz, DMSO-d₆): 8 8.55 (s, 1 H), 7.22 (s, 1 H), 7.16 (s, 1 H), 6.58 (bs, 2 H), 6.21 (t, 1 H), 3.92 (s, 3 H), 3.91 (s, 3 H), 3.85 (d, J=5.2 Hz, 2 H), 3.66-3.64 (m, 8 H); LC-MS (ES) m/z = 410.45 [M]⁺; HPLC purity: 99.38 %.

Example 53: Synthesis of N-(4-((6,7-dimethoxyquinolin-4-yl)oxy)phenyl)cyclopropanesulfonamide:

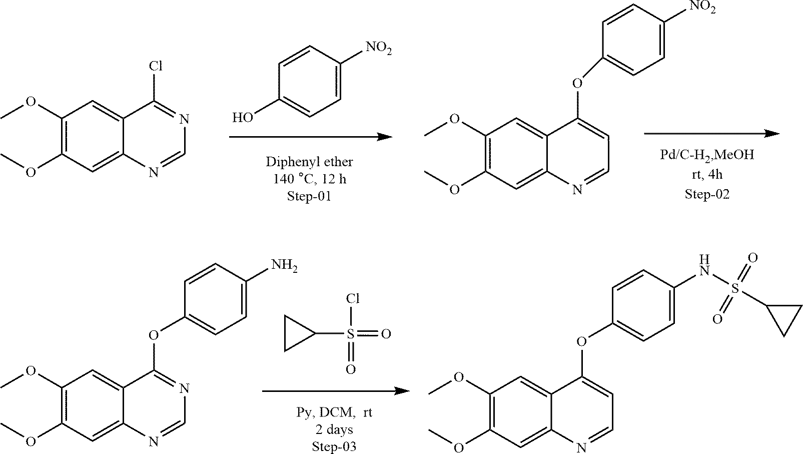

### Step 1: 6,7-Dimethoxy-4-(4-Nitrophenoxy)Quinoline

Procedure: To a stirred solution of 4-chloro-6,7-dimethoxyquinoline (2.5 g, 11.177 mmol) in diphenyl ether (30 mL) was added 4-nitrophenol (1.7 g, 12.29 mmol). The resulting mixture was stirred at 140° C. for 12 h. The progress of the reaction was monitored by TLC. After completion of the reaction, the reaction mixture was cooled to 0° C.; the solid formed was washed with n-hexane and diethyl ether. The precipitate was filtered and dried under vacuum to afford 6,7-dimethoxy-4-(4-nitrophenoxy)quinoline as off- white solid (3.22 g, crude). The crude compound was taken for next step without purification. LC-MS (ES) m/z = 327.0 [M+H]⁺.

### Step 2: 4-((6,7-Dimethoxyquinolin-4-yl)Oxy)Aniline

Procedure: To a stirred solution of 6,7-dimethoxy-4-(4-nitrophenoxy)quinoline (3.22 g, 9.86 mmol) in methanol (20 mL) was added 10 % palladium on carbon. The resulting mixture was stirred at room temperature for 4 h. The progress of the reaction was monitored by TLC. After completion of the reaction, the reaction mixture was filtered through celite, the filtrate was evaporated under reduced pressure to afford 4-((6,7-dimethoxyquinolin-4-yl)oxy)aniline as an off-white solid (1.95 g, crude). The crude compound obtained was taken for next step without purification.LC-MS (ES) m/z = 297.3 [M+H]⁺ .

### Step3: N-(4-((6,7-Dimethoxyquinolin-4-yl)Oxy)Phenyl)Cyclopropanesulfonamide

Procedure: To a stirred solution of 4-((6,7-dimethoxyquinolin-4-yl)oxy)aniline (1.4 g, 4.72 mmol) in dichloromethane (20 mL) was added pyridine (3.04 mL, 37.79 mmol) at room temperature and stirred for 10 min. Cyclopropanesulfonyl chloride (2.4 mL, 23.62 mmol) was added. The resulting mixture was stirred at room temperature for 2 days. The progress of the reaction was monitored by TLC. The reaction mixture was evaporated under reduced pressure, the reddish crude product was diluted with dichloromethane (100 mL) and washed with water (2 × 50 mL), combined organic layers were separated, dried over anhydrous sodium sulphate, filtered and evaporated under reduced pressure to give crude product. The crude product was purified by combiflash chromatography using silica gel column with methanol in DCM as eluents. Desired product was eluted in 8 to 10 % methanol in DCM to afford N-(4-((6,7-dimethoxyquinolin-4-yl)oxy) phenyl) cyclopropanesulfonamide as reddish solid (1 g, 48 %). The solid was washed with diethyl ether (2 × 5 mL) and then dried for 30 minutes at 50° C.1H NMR (400 MHz, DMSO-d₆): 8 9.80 (s, 1 H), 8.53 (bs, 1 H), 7.53 (s, 1 H), 7.40 - 7.35 (m, 3 H), 7.26 (d, J= 8.4 Hz, 2 H), 6.53 (d, 1 H), 3.95 (s, 3 H), 3.93 (s, 3 H), 2.05 (bs, 1 H), 0.95 - 0.93 (m, 4 H).LC-MS (ES) m/z = 401.3 [M+H]⁺, HPLC purity: 99.7 %. at 254 nm.

Example 54: N-(4-((7-methoxyquinolin-4-yl)oxy)phenyl)sulfamide:

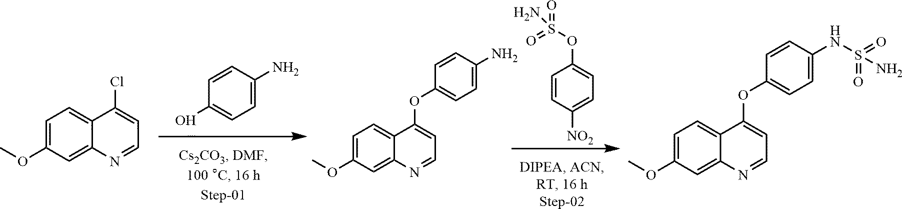

### Step 1 4-((7-Methoxyquinolin-4-yl)Oxy)Aniline

Procedure: To a stirred solution of 4-chloro-7-methoxyquinoline (0.5 g, 2.58 mmol), 4-aminophenol (0.30 g, 2.84 mmol) in dry DMF (5 mL) was added cesium carbonate (2.52 g, 7.74 mmol) and stirred for 16 h at 100° C. The progress of the reaction was monitored by TLC. The reaction mixture was cooled to room temperature and was slowly added drop wise to cold water (~10° C., 4 L) under stirring. Grey color solid precipitated out and the stirring continued for 1h at room temperature. The solid was filtered and was washed with DM water (2 x 50 mL). The solid was dried under vacuum for 2 h. The obtained solid was then dissolved in 10 % MeOH/DCM (20 mL). The organic layer was separated, dried over anhydrous sodium sulphate and evaporated to give crude product as brown solid. The crude product was purified using 100-200 silica gel with methanol/dichloromethane as eluents. Desired product eluted in 2 to 2.5 % methanol/dichloromethane. The combined fractions were evaporated to afford 4-((7-methoxyquinolin-4-yl)oxy)aniline as off-white solid (0.5 g, 72.46%). ¹H NMR (400 MHz, DMSO-d₆): δ 8.53 (d, J= 5.2 Hz, 1 H), 8.17 (d, J= 9.2 Hz, 1 H), 7.35 (d, J= 1.6 Hz, 1 H), 7.24-7.21 (m, 1 H), 6.90 (d, J = 8.8 Hz, 2 H), 6.64 (d, J = 8.4 Hz, 2 H), 6.36 (d, J = 5.2 Hz, 1H), 5.11 (br s, 2 H), 3.90 (s, 3H).LC-MS (ES) m/z = 267.1[M+H]⁺ .

### Step 2 N-((7-Methoxyquinolin-4-yl)Oxy)Phenyl)Sulfamide

Procedure: To a stirred solution of 4-((7-methoxyquinolin-4-yl) oxy) aniline (0.5 g, 1.87 mmol) and 4-nitrophenyl sulfamate (0.61 g, 2.81 mmol) in acetonitrile (10 mL) was added DIPEA (1.63 mL, 0.0093 mmol) at room temperature. The reaction mixture was stirred for 16 h at room temperature. The progress of the reaction was monitored by TLC. The yellow precipitate formed was filtered, washed with acetonitrile (10 mL) followed by diethyl ether (2 x 20 mL). The obtained pale yellow solid was triturated from ethyl acetate. The solid was dried under vacuum for 2 h to afford N-(4-((7-methoxyquinolin-4-yl)oxy)phenyl)sulfamide as a white solid (0.58 g, 89.4 %). ¹H NMR (400 MHz, DMSO-d₆): 8 9.56 (s, 1H), 8.57 (d, J = 5.2 Hz, 1 H), 8.18 (d, J = 9.2 Hz, 1 H), 7.37 (s, 1 H), 7.28-7.25 (m, 2 H), 7.19 (d, J = 8.8 Hz, 2 H), 7.18 (s, 2 H), 6.40 (d, J = 5.2 Hz, 1 H), 3.91 (s, 3 H). ¹H NMR (400 MHz, DMSO-d₆): 8 9.56 (s, 1 H), 8.57 (d, J= 5.2 Hz, 1 H), 8.18 (d, J= 9.2 Hz, 1 H), 7.37 (s, 1 H), 7.28-7.25 (m, 3 H), 7.19 (d, J= 8.8 Hz, 2 H), 7.18 (s, 2 H), 6.40 (d, = 5.2 Hz, 1 H), 3.91 (s, 3 H).LC-MS (ES) m/z = 345.9 [M+H]⁺, HPLC purity 99.00% at 254 nm.

Example 55: N-(4-(7-methoxyquinolin-4-yl)benzyl)sulfamide:

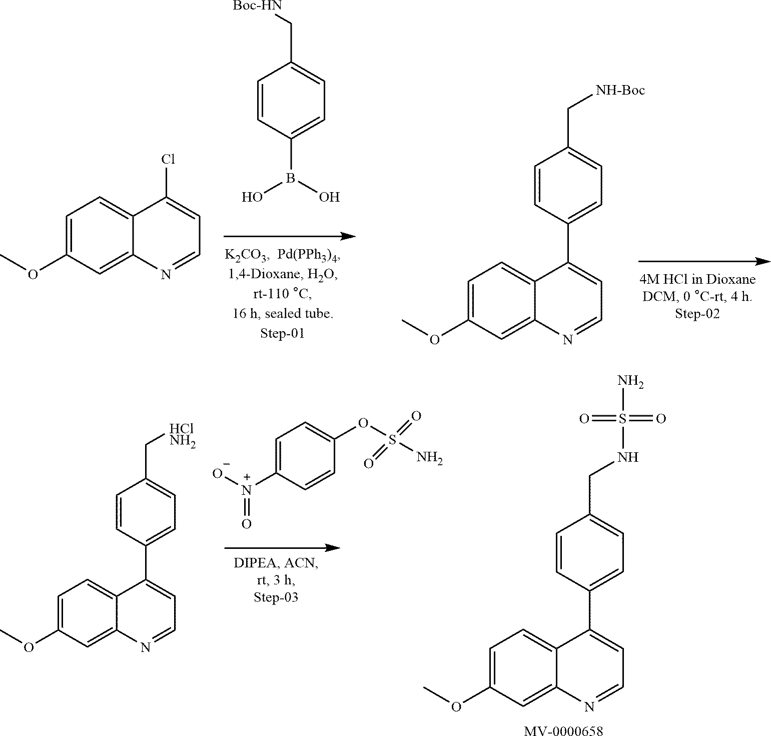

### Step 1 Tert-Butyl (4-(7-Methoxyquinolin-4-yl)Benzyl)Carbamate

Procedure: To a stirred solution of 4-chloro-7-methoxyquinoline (0.1 g, 0.518 mmol), (4-(((tert-butoxycarbonyl)amino)methyl)phenyl)boronic acid (0.130 g, 0.518 mmol) in dioxane and water (5 mL) was added potassium carbonate (0.179 g, 1.295 mmol).The mixture was purged with argon gas for 15 minutes, then added tetrakis(triphenylphosphine)palladium(0) (0.030 g, 0.025 mmol) at room temperature. Reaction mixture was stirred for 16 h at 110° C. Progress of the reaction was monitored by TLC. Reaction mixture was cooled to room temperature, reaction mixture was filtered through celite and filtrate was concentrated under reduced pressure to give crude compound. The crude compound was purified by combiflash purifier using 2% methanol in dichloromethane as eluent to afford tert-butyl (4-(7-methoxyquinolin-4-yl)benzyl)carbamate as off-white solid (0.120 g, 64%). LC-MS (ES) m/z = 365.1 [M+H] ⁺.

### Step 2 (7-Methoxyquinolin-4-yl)Phenyl)Methanamine

Procedure: To a stirred solution of tert-butyl (4-(7-methoxyquinolin-4-yl)benzyl)carbamate (0.150 g, 0.411 mmol) in DCM (10 mL), was added 4M HC1 in Dioxane (2 mL) at 0° C. Reaction mixture was stirred for 4 h at room temperature. Progress of the reaction was monitored by TLC. Reaction mixture was concentrated under reduced pressure to give the crude compound, The crude product was washed with diethyl ether and n-pentane to afford (4-(7-methoxyquinolin-4-yl) phenyl)methanamine.HCl as an off-white solid (0.110 g, 60%). LC-MS (ES) m/z = 265.1 [M+H] ⁺.

### Step 3 N-(7-Methoxyquinolin-4-yl) Benzyl)Sulfamide

Procedure: To a stirred solution of (4-(7-methoxyquinolin-4-yl)phenyl)methanamine hydrochloride (0.1 g, 0.333 mmol) in acetonitrile (10 mL) was added diisopropylethylamine (0.11 mL, 0.66 mmol) and stirred for 5 minutes. Then 4-nitrophenyl sulfamate (0.080 g, 0.366 mmol) was added at room temperature, and stirred for 3 h. The progress of the reaction was monitored by TLC. The reaction was quenched with water and the compound was extracted with ethyl acetate (2 × 10 mL), combined organic layer were dried over sodium sulphate, filtered and evaporated under reduced pressure to give the crude compound. The obtained crude product was purified by combiflash purifier using 4% methanol in dichloromethane as eluent to afford N-(4-(7-methoxyquinolin-4-yl)benzyl)sulfonic amide as an off-white solid (0.050 g, 44%). ¹HNMR (400 MHz, DMSO-d₆): δ 8.83(d, J= 4.0 Hz, 1 H), 7.74(d, J= 9.2 Hz, 1 H), 7.54 - 7.46 (m, 5 H), 7.27 - 7.22 (m, 2 H), 7.15 (s, 1 H), 6.66 (s, 2 H), 4.17 (d, J= 6.0 Hz, 2 H), 3.92 (s, 3 H). LC-MS (ES) m/z = 344.2[M-H]⁺, HPLC purity 99.71% at 220 nm.

Example 56: 4-(((6-fluoro-7-methoxyquinolin-4-,yl)amino)methyl)benzenesulfonamide:

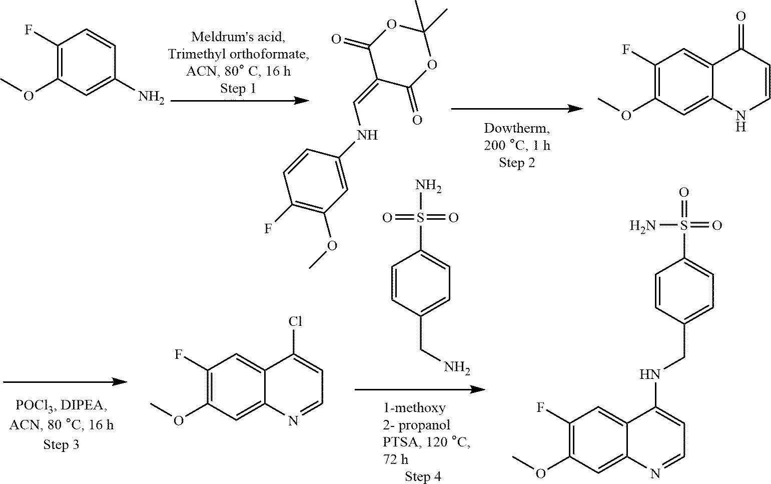

### Step 1: 5-(((4-Fluoro-3-Methoxyphenyl)Amino)Methylene)-2,2-Dimethyl-1,3-Dioxane-4,6-Dione

Procedure: To a stirred solution of 4-fluoro-3-methoxyaniline (3 g, 21.25 mmol) in ACN (30 mL), Meldrum’s acid (3.9 g, 27.63 mmol) and trimethyl orthoformate (3.25 mL, 29.75 mmol) were added at room temperature. The resulting mixture was heated to 80° C. for 16 h. The progress of the reaction was monitored by TLC. Reaction mixture was cooled to room temperature and evaporated the solvent under reduced pressure. The crude product was triturated with n-pentane (3 x 50 mL) and decanted, dried to afford 5-(((4-fluoro-3-methoxyphenyl)amino)methylene)-2,2-dimethyl-1,3-dioxane-4,6-dione as brown solid (6 g , crude). LC-MS (ES) m/z = 293.9 [M-H]⁺.

### Step 2: 6-Fluoro-7-Methoxyquinolin-4(IH)-One

Procedure: To a stirred solution of 5-(((4-fluoro-3-methoxyphenyl)amino)methylene)-2,2-dimethyl-1,3-dioxane-4,6-dione (6.5 g, 22.01 mmol) in Dowtherm, (150 mL) was heated to 200° C. for 1 h. The progress of the reaction was monitored by TLC. Reaction mixture was cooled to room temperature and triturated with n-pentane (3 x 70 mL) and decanted, dried to afford 6-fluoro-7-methoxyquinolin-4(1H)-one (3 g, crude) as brown solid. LC-MS (ES) m/z = 194.1 [M+H]⁺.

### Step 3: 4-Chloro-6-Fluoro-7-Methoxyquinoline

Procedure: To a stirred solution of 6-fluoro-7-methoxyquinolin-4(1H)-one (3 g, 15.54 mmol) in ACN (100 mL), DIPEA (6 mL) at 0° C. was added POC1₃ (15 mL). The resulting reaction mixture was heated to 80° C. for 16 h. The progress of the reaction was monitored by TLC. Evaporated the solvent under reduced pressure and diluted with ice cold water (50 mL) and neutralized with NaHCO₃ and extracted with ethyl acetate (3 × 50 mL). The combined organic layers were dried over anhydrous sodium sulphate, filtered and evaporated under reduced pressure, purified by combiflash purifier using 15-20% EtOAc in hexane to give 4-chloro-6-fluoro-7-methoxyquinoline as off- white solid(1.1 g, 34.37%).¹HNMR (400 MHz, DMSO-d₆): 8 8.67 (d, J= 4.4 Hz, 1H), 7.85 (d, J= 11.2 Hz, 1H), 7.53 (d, J = 8 Hz, 1 H), 7.39 (d, J= 4.8 Hz, 1 H), 4.04 (s, 3H). LC-MS (ES) m/z = 212.1 [M+H]⁺.

### Step 4: 4-(((6-Fluoro-7-Methoxyquinolin-4-yl)Amino)Methyl)Benzenesulfonamide

Procedure: To a stirred solution of 4-chloro-6-fluoro-7-methoxyquinoline (0.6 g, 2.843 mmol), 4-(aminomethyl)benzenesulfonamide (0.68 g, 3.696 mmol) in 1-methoxy 2-propanol (25 mL) was added PTSA (0.24 g, 1.421 mmol) at room temperature. The resulting mixture was heated to 120° C. for 72 h. The progress of the reaction was monitored by TLC. The reaction mixture was evaporated under reduced pressure to give crude product. The crude compound was purified by combiflash purifier using 3-5% methanol in dichloromethane. Fractions containing compound were concentrated and resulting solid was dissolved in 10% methanol in dichloromethane (300 mL), washed with water (50 mL). Organic layer evaporated under reduced pressure and triturated with ACN (2 x 20 mL) to give 4-(((6-fluoro-7-methoxyquinolin-4-yl)amino)methyl)benzenesulfonamide as off-white solid (0.16 g , 16%). ¹HNMR (400 MHz, DMSO-d₆): 8 8.21 (d, J= 5.2 Hz, 1 H), 8.10 (d, J= 13.6 Hz, 1 H), 7.76-7.74 (m, 3 H), 7.52 (d, J= 8.4 Hz, 2 H), 7.33 (d, J= 8.4 Hz, 1 H), 7.25 (s, 2 H), 6.19 (d, J= 5.6 Hz, 1 H), 4.57 (d, J= 5.6 Hz, 2 H), 3.93 (s, 3 H). LC-MS (ES) m/z =362.0 [M+H]⁺. HPLC purity 99.11% at 254 nm.

Example 57: 8-(3-cyano-6-fluoro-7-methoxyquinolin-4-yl)-2,8-diazaspiro[4.5]decane-2-sulfonamide:

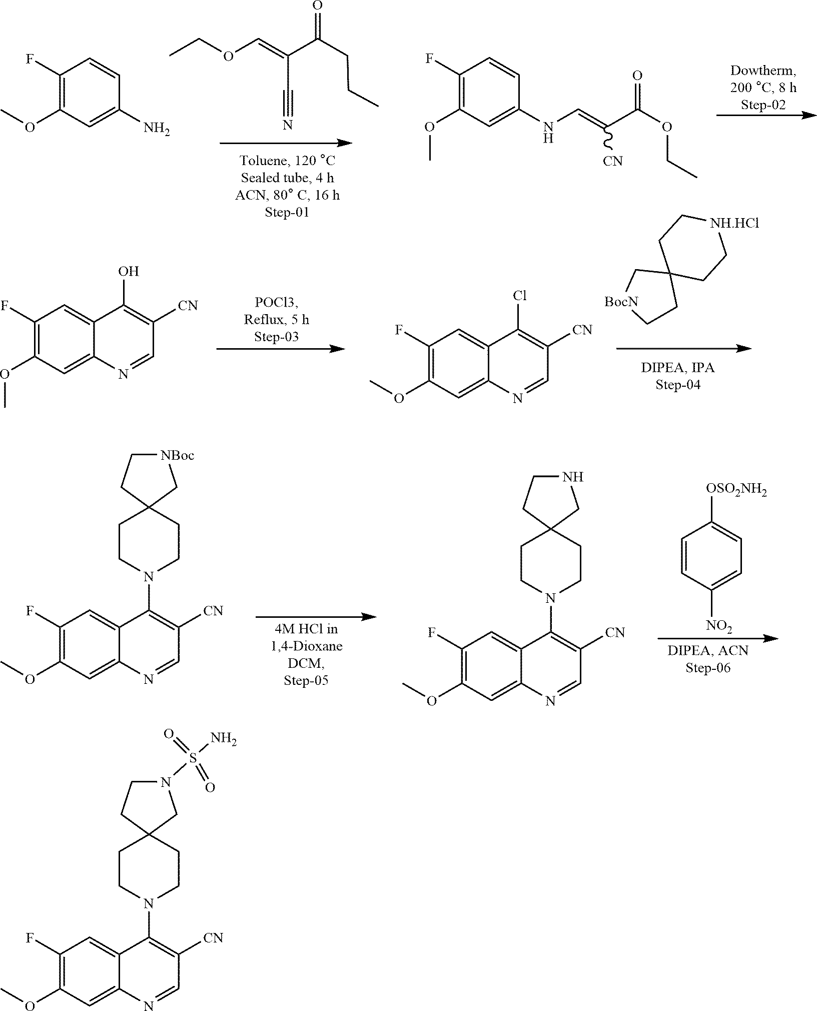

### Step 1: Ethyl 2-Cyano-3-((4-Fluoro-3-Methoxyphenyl) Amino) Acrylate

Procedure: To a stirred solution of 4-fluoro-3-methoxyaniline (1.0 g, 7.09 mmol) in toluene (10 mL) was added ethyl 2-cyano-3-((4-fluoro-3-methoxyphenyl)amino)acrylate (1.2 g, 7.09 mmol) in a sealed tube. The reaction mixture was stirred at 120° C. for 4 h. The reaction mixture was cooled to room temperature and the precipitated solid was filtered under vacuum. The residue was thoroughly washed with toluene and dried under vacuum to obtain ethyl 2-cyano-3-((4-fluoro-3-methoxyphenyl)amino)acrylate as a mixture of E and Z isomers as brown solid (1.8 g, 95 %). ¹HNMR (400 MHz, DMSO-d₆): δ 10.69 - 10.67 (m, 2 H), 8.46 (d, J= 14.4 Hz, 1 H), 8.29 (s, 1 H), 7.37 (d, J= 6.8 Hz, 1 H), 7.21 - 7.18 (m, 3 H), 7.0 - 6.93 (m, 2 H), 4.21 - 4.14 (m, 4 H), 3.84 (s, 3 H), 3.83 (s, 3 H), 1.26 -1.19 (m, 6 H). LC-MS (ES) m/z = 265.1 [M+H]⁺

### Step 2: 6-Fluoro-4-Hydroxy-7-Methoxyquinoline-3-Carbonitrile

Procedure A stirred solution of ethyl 2-cyano-3-((4-fluoro-3-methoxyphenyl)amino)acrylate (1.8 g, 6.81 mmol) in Dowtherm (20.0 ml) was heated to 250° C. for 6 hours. The reaction mixture was cooled to room temperature and n-hexane was added and stirred for 30 minutes. The precipitated solid was filtered under vacuum. The residue was thoroughly washed with n-hexane and dried under vacuum to obtain 6-fluoro-4-hydroxy-7-methoxyquinoline-3-carbonitrile as brown solid (1.0 g crude).LC-MS (ES) m/z = 219.1 [M+H]⁺.

### Step 3: 4-Chloro-6-Fluoro-7-Methoxyquinoline-3-Carbonitrile

Procedure: A stirred solution of 6-fluoro-4-hydroxy-7-methoxyquinoline-3-carbonitrile (1.0 g, 4.58 mmol) in POC1₃ (10.0 ml) was heated to 100° C. for 6 hours. The reaction mixture was cooled to room temperature and completely evaporated to obtain crude compound which was basified with sat. sodium bicarbonate solution and extracted with ethyl acetate. The organic layer was dried over sodium sulphate and evaporated off to obtain 4-chloro-6-fluoro-7-methoxyquinoline-3-carbonitrile (0.5 g, 46 %) as yellow solid. ¹HNMR (400 MHz, DMSO-d₆): 8 9.11 (s, 1 H), 8.07 (d, J= 11.6 Hz, 1 H), 7.76 (d, J= 8.0 Hz, 1 H), 4.14 (s, 3 H).

### Step 4: Tert-Butyl 8-(3-Cyano-6-Fluoro-7-Methoxyquinolin-4-yl)-2,8-Diazaspiro[4.5]Decane-2-Carboxylate

Procedure: To a stirred solution of 4-chloro-6-fluoro-7-methoxyquinoline-3-carbonitrile (0.3 g, 1.27 mmol) in IPA (10.0 ml) was added tert-butyl 2, 8-diazaspiro [4.5] decane-2-carboxylate hydrochloride (0.39 g, 1.40 mmol) followed by DIPEA (0.65 mL, 3.81 mmol). The reaction mixture was heated at 90° C. in a sealed tube for 16 hours. The reaction mixture was cooled to room temperature and evaporated to obtain crude compound which was purified over silica gel flash column chromatography. The compound was eluted in 50 % EtOAc:Hexane. Pure fractions were evaporated to obtain tert-butyl 8-(3-cyano-6-fluoro-7-methoxyquinolin-4-yl)-2,8-diazaspiro[4.5]decane-2-carboxylate (0.3 g, 54 %) as off-white solid. ¹HNMR (400 MHz, DMSO-d₆): δ 8.67 (s, 1 H), 7.73 (d, J= 12.4 Hz, 1 H), 7.54 (d, J= 8.4 Hz, 1 H), 4.0 (s, 3 H), 3.6 - 3.5 (m, 4 H), 3.4 - 3.3 (m, 2 H), 3.20 - 3.15 (m, 2 H), 1.81 - 1.74 (m, 6 H), 1.39 (s, 9HLC-MS (ES) m/z = 441.2 [M+H]⁺

### Step 5: Synthesis of 6-Fluoro-7-Methoxy-4-(2,8-Diazaspiro[4.5]Decan-8-yl)Quinoline-3-Carbonitrile

Procedure: To a stirred solution of tert-butyl 8-(3-cyano-6-fluoro-7-methoxyquinolin-4-yl)-2,8-diazaspiro[4.5]decane-2-carboxylate (0.3 g, 0.681 mmol) in DCM (10.0 ml) was added 4M HC1 in 1,4-Dioxane (10 mL) at 0° C. . The reaction mixture was warmed to room temperature and stirred for 16 hours. Solvents were completely evaporated to obtain crude compound, which was triturated in n-pentane and filtered under vacuum. The residue was thoroughly washed and dried under vacuum to obtain 6-fluoro-7-methoxy-4-(2,8-diazaspiro[4.5]decan-8-yl)quinoline-3-carbonitrile (0.2 g, 87 %) as yellow solid. ¹HNMR (400 MHz, DMSO-d₆): δ 9.22 (s, 1 H), 8.83 (s, 1 H), 7.80 (d, J= 12.8 Hz, 1 H), 7.59 (d, J= 8.4 Hz, 1 H), 4.01 (s, 3 H), 3.68 - 3.65 (m, 4 H), 3.29 - 3.26 (m, 2 H), 3.12 - 3.09 (m, 2 H), 1.96 - 1.81 (m, 6 H). LC-MS (ES) m/z = 341.4 [M+H]⁺ .

### Step 6: 8-(3-Cyano-6-Fluoro-7-Methoxyquinolin-4-yl)-2,8-Diazaspiro[4.5]Decane-2-Sulfonamide

Procedure: To a stirred solution of 6-fluoro-7-methoxy-4-(2,8-diazaspiro[4.5]decan-8-yl)quinoline-3-carbonitrile ( 0.2 g, 0.58 mmol) in acetonitrile (10 mL) was added DIPEA (0.3 mL, 1.76 mmol) and stirred for 5 min at room temperature. 4-nitrophenyl sulfamate (0.13 g, 0.58 mmol) was added and stirred for 16 h at room temperature. The progress of the reaction was monitored by TLC. The reaction mixture was completely evaporated to obtain crude compound which was purified over silica gel flash column chromatography. The compound eluted in 3 % MeOH : DCM. Pure fractions were evaporated to obtain 8-(3-cyano-6-fluoro-7-methoxyquinolin-4-yl)-2,8- diazaspiro[4.5]decane-2-sulfonamide as off-white solid (0.07 g, 24 %). ¹HNMR (400 MHz, DMSO-d₆): δ 8.68 (s, 1 H), 7.72 (d, J = 12.8 Hz, 1 H), 7.55 (d, J= 8.4 Hz, 1 H), 6.73 (s, 2 H), 4.0 (s, 3 H), 3.56 - 3.55 (m, 4 H), 3.22 (t, J= 7.2 Hz, 2 H), 3.10 (s, 2 H), 1.85 - 1.78 (m, 6 H). LC-MS (ES) m/z = 420.3 [M+H]⁺. HPLC purity: 98.62 % at 254 nm.

Example 58: 4-((7-methoxyquinolin-4-yl)oxy)benzenesulfonamide:

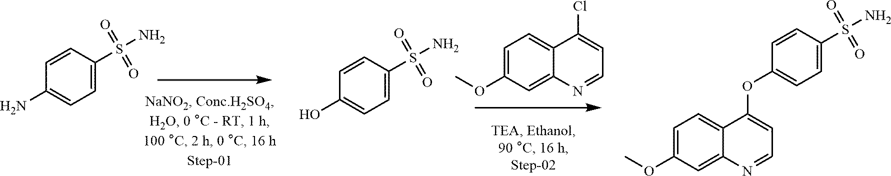

### Step1 4-Hydroxybenzenesulfonamide

Procedure: To a stirred solution of 4-aminobenzenesulfonamide (2.0 g, 0.0116 mmol) in water (16 mL) and Conc. H₂SO₄ (8 mL) at 0° C., sodium nitrite (0.8 g, 0.0116 mmol) was added and stirred at room temperature for 1 h. Then the reaction mixture was heated to 100° C. for 2 h. The reaction mixture was cooled to room temperature and then kept at 0° C. (in refrigerator) for overnight. The crystals formed was filtered and washed with diethyl ether. The filtrate obtained was extracted with diethyl ether (3 × 25 mL) and dried over sodium sulphate, solvent was evaporated under reduced pressure to afford 4-hydroxybenzenesulfonamide (1.0 g) as pale-yellow solid. The solid obtained was acidified with saturated citric acid solution and extracted with ethyl acetate (3 × 25 mL). The organic layers were combined and dried over sodium sulphate. The solvent was evaporated under reduced pressure to afford 4-hydroxybenzenesulfonamide (1.6 g, 80 %) as pale yellow solid.¹HNMR (400 MHz, DMSO-d₆): δ 10.21(bs, 1 H), 7.62 (d, J= 8.4 Hz, 2 H), 7.07(bs, 2 H), 6.85 (d, J= 8.0 Hz, 2 H).

### Step2 4-((7-Methoxyquinolin-4-yl)Oxy)Benzenesulfonamide

Procedure: To a stirred solution of 4-hydroxybenzenesulfonamide (1 g, 0.0057 mmol) in ethanol (15 mL) was added triethylamine (3.2 mL, 0.0231 mmol) and stirred for 5 min. followed by 4-chloro-7-methoxyquinoline (1.1 g, 0.00578 mmol) was added and stirred for 16 h at 90° C. The progress of the reaction was monitored by TLC. The solvent was evaporated under reduced pressure to get the crude compound. The crude residue was purified by combiflash using methanol in dichloromethane (1-3.5%) to afford 4-((7-methoxyquinolin-4-yl)oxy)benzenesulfonamide (48.5 mg , 2.5 %) as off- white solid. ¹HNMR (400 MHz, DMSO-d₆): δ 8.67(d, J= 5.2 Hz, 1 H), 8.1(d, J= 9.2 Hz, 1 H), 7.91(d, J = 8.8 Hz, 2 H), 7.43-7.39(m, 5 H), 7.29-7.26 (m, 1 H), 6.65(d, J= 5.2 Hz, 1 H), 3.92(s, 3H). LC-MS (ES) m/z = 331.0 [M+H]⁺ .HPLC Purity- 99.95% at 254 nm.

Example 59: N-(1-(7-methoxyquinolin-4-yl)indolin-5-yl)sulfamide:

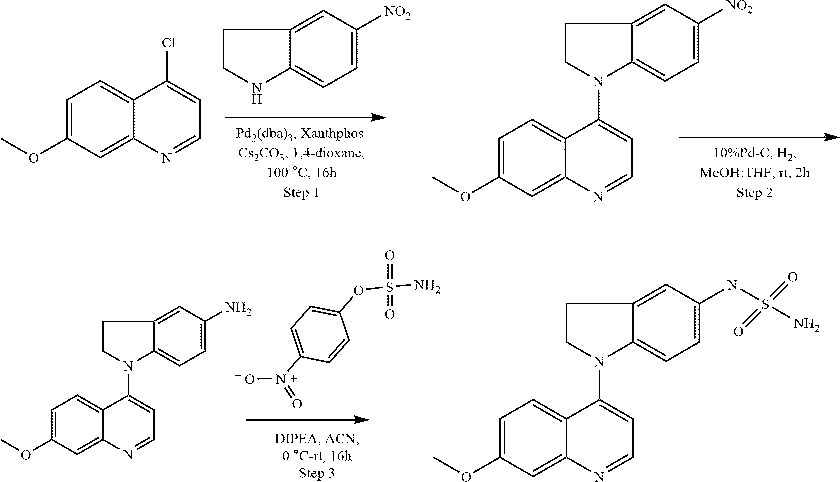

### Step 1 7-Methoxy-4-(5-Nitroindolin-1-yl) Quinoline

Procedure: To a stirred solution of 4-chloro-7-methoxyquinoline (0.05 g, 0.25 mmol) in 1,4-dioxane (5 mL) were added 5-nitroindoline (0.046 g, 0.28 mmol), cesium carbonate (0.25 g, 0.77 mmol) and Xanthphos (0.029 g, 0.051 mmol). The resulting mixture was purged with argon for 15 min and added Pd₂(dba)₃ (0.023 g, 0.025 mmol) was added and the mixture was purged for another 10 min. Resulting mixture was stirred for 16 h at 100° C. Progress of the reaction was monitored by TLC. Reaction mixture was filtered through celite bed and filtrate was diluted with ethyl acetate (20 mL), washed with water (2 x 10 mL) followed by brine (10 mL), dried over anhydrous sodium sulphate, filtered and evaporated under reduced pressure. The crude was purified by combiflash using 55-60% ethyl acetate in hexane to afford 7-methoxy-4-(5-nitroindolin-1-yl)quinoline as yellow solid (0.12 g, 75%). ¹HNMR (400 MHz, DMSO-d₆): δ 8.83 (d, J= 4.4 Hz, 1 H), 8.08 (s, 1 H), 7.91 (d, J= 8.8 Hz, 1 H), 7.73 (d, J= 9.2 Hz, 1 H), 7.46 (s, 1 H), 7.36 (d, J= 4.8 Hz, 1 H), 7.23 (d, J= 9.2 Hz, 1 H), 6.24 (d, J= 8.8 Hz, 1 H), 4.26 (t, J= 8.4 Hz, 2 H), 3.93 (s, 3 H), 3.32 (t, J= 8.4 Hz, 2 H); LCMS (ES) m/z = 322.1 [M+H]⁺.

### Step2: 1-(7-Methoxyquinolin-4-yl)Indolin-5-Amine

Procedure: To a stirred solution of 7-methoxy-4-(5-nitroindolin-1-yl)quinoline (0.12 g, 0.37 mmol) in methanol : THF (5:5 mL) was added 10% palladium on carbon (0.05 g) and the resulting reaction mixture was stirred for 2 h at room temperature under hydrogen atmosphere. Progress of the reaction was monitored by TLC. The reaction mixture was filtered, organic layer was concentrated under reduced pressure to afford 1-(7-methoxyquinolin-4-yl)indolin-5-amine as yellow solid (0.07 g, 64%). ¹HNMR (400 MHz, DMSO-d₆): δ 8.54 (s, 1 H), 7.85 (d, J= 9.2 Hz, 1 H), 7.30 (s, 1 H), 7.10 (d, J= 8.8 Hz, 1 H), 7.01 (s, 1 H), 6.56 (s, 1 H), 6.35 (d, J= 8.0 Hz, 1 H), 6.26 (d, J= 8.0 Hz, 1 H), 4.69 (s, 2 H), 3.97 (t, J= 7.2 Hz, 2 H) 3.89 (s, 3 H), 3.00 (t, J= 6.8 Hz, 2 H); LCMS (ES) m/z = 292.1 [M+H]⁺.

### Step 3: N-(1-(7-Methoxyquinolin-4-yl)INDOLIN-5-Yl)Sulfamide

Procedure: To a stirred solution of 1-(7-methoxyquinolin-4-yl)indolin-5-amine (0.07 g, 0.24 mmol) in acetonitrile (10 mL) was added 4-nitrophenyl sulfamate (0.062 g, 0.28 mmol), and N,N-Diisopropylethylamine (0.12 mL, 0.72 mmol) at 0° C. Reaction mixture was stirred at room temperature for 16 h, reaction was monitored by TLC. The reaction mixture was evaporated under reduced pressure. The crude residue was purified by prep. HPLC using ZORBAX XDB C18 (150 mmX4.6 mmX5 um) column with 0.1% ammonia in water/ACN as mobile phases to afford N-(1-(7-methoxyquinolin-4-yl)indolin-5-yl)sulfamide as pale yellow solid (0.048 g, 54%). ¹HNMR (400 MHz, DMSO-d₆): δ 8.96 (s, 1 H), 8.64 (d, J= 4.8 Hz, 1 H), 7.81 (d, J= 9.2 Hz, 1 H), 7.36 (s, 1 H), 7.14 - 7.12 (m, 3 H), 6.84 (s, 1 H), 6.79 (d, J= 8.4 Hz, 2H), 6.36 (d, J= 8.4, Hz, 1H), 4.03 (t, J= 7.6 Hz, 2 H), 3.91 (s, 3 H), 3.13 (t, J= 8.0 Hz, 2 H); LCMS (ES) m/z = 368.9 [M-H]⁺; HPLC purity: 99.52%.

Example 60: 7-(6,7-dimethoxy-3-methylquinolin-4-yl)-3,4-dihydroisoquinoline-2(1H)-sulfonamide:

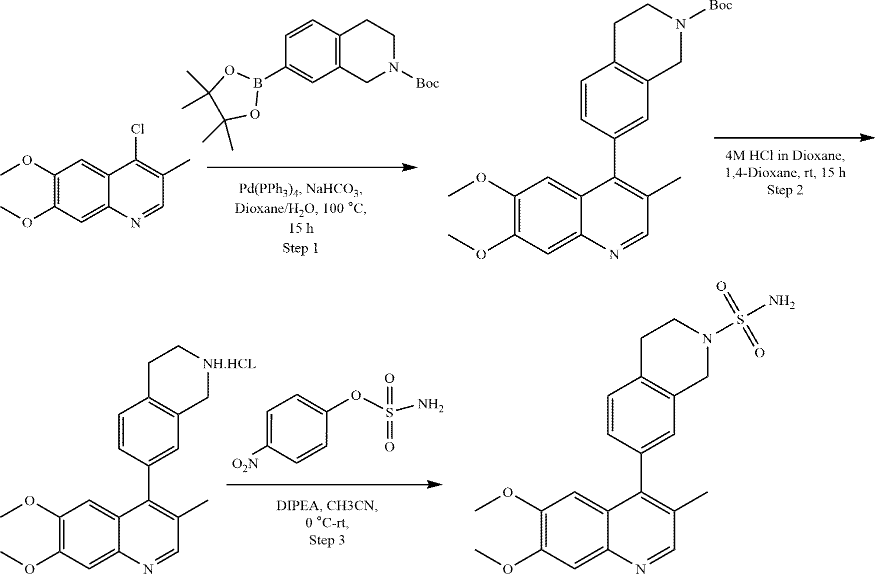

### Step 1: Tert-Butyl 7-(6,7-Dimethoxy-3-Methylquinolin-4-yl)-3,4-Dihydroisoquinoline-2(1H)-Carboxylate

Procedure: To a stirred solution of 4-chloro-6,7-dimethoxy-3-methylquinoline (0.19 g, 0.799 mmol) in 1,4-dioxane (20 mL) and water (4 mL) was added tert-butyl 7-(4,4,5,5-tetramethyl-1,3,2-dioxaborolan-2-yl)-3,4-dihydroisoquinoline-2(1H)-carboxylate (0.31 g, 0.879 mmol), K₂CO₃ (0.33 g, 2.397 mmol) and the reaction mixture was degassed with argon for 10 min in a sealed tube. To this reaction mixture was added Pd(PPh₃)₄ (0.09 mL, 0.079 mmol) and heated to 100° C. for 15 h. The progress of the reaction was monitored by TLC. Reaction mixture was cooled to room temperature, ethyl acetate (100 mL) was added. Organic layer was separated and washed with water, brine and dried over anhydrous sodium sulphate. The filtrate obtained was evaporated under reduced pressure to give the crude residue. The crude product was purified by gradient column chromatography using 1-3% methanol in DCM to afford tert-butyl 7-(6,7-dimethoxy-3-methylquinolin-4-yl)-3,4-dihydroisoquinoline-2(1H)-carboxylate as colorless viscous liquid (0.145 g, crude). LC-MS (ES) m/z = 435.2[M+H]⁺.

### Step 2: Synthesis of 6,7-Dimethoxy-3-Methyl-4-(1,2,3,4-Tetrahydroisoquinolin-7-yl)Quinoline Hydrochloride

Procedure: To a stirred solution of tert-butyl 7-(6,7-dimethoxy-3-methylquinolin-4-yl)-3,4-dihydroisoquinoline-2(1H)-carboxylate (0.145 g, 0.334 mmol) in 1,4-Dioxane (1 mL) was added 4 M HC1 in dioxane (3 mL). The reaction mixture was stirred at room temperature for 15 h. The progress of the reaction was monitored by TLC. The solvent was evaporated and the residue was washed with diethyl ether followed by pentane and dried under reduced pressure to afford 6,7-dimethoxy-3-methyl-4-(1,2,3,4-tetrahydroisoquinolin-7-yl)quinoline hydrochloride as off-white solid (0.1 g, 84%). LC-MS (ES) m/z = 335.1[M+H]⁺.

### Step 3: 7-(6,7-Dimethoxy-3-Methylquinolin-4-yl)-3,4-Dihydroisoquinoline-2(1H)-Sulfonamide

Procedure: To a stirred solution of 6,7-dimethoxy-3-methyl-4-(1,2,3,4-tetrahydroisoquinolin-7-yl)quinoline hydrochloride (0.1 g, 0.2701 mmol) and 4-nitrophenyl sulfamate (0.076 g, 0.351 mmol) in acetonitrile (10 mL) was added N,N-diisopropyl ethylamine (0.14 mL, 0.810 mmol) at 0° C. The reaction mixture was stirred at room temperature for 15 h. The progress of the reaction was monitored by TLC. The Solvent was concentrated, and the obtained residue was diluted with water and extracted with DCM (3 x 30 mL). The organic layer was dried over sodium sulphate and concentrated. The crude product was purified by gradient column chromatography using 12% methanol in dichloromethane. Fractions containing compound were pooled and concentrated and further purified by Prep-HPLC (Inertsil ODS 3 V(150 mm×4.6 mm×5 mic), Mobile phase(A):0.1 % Ammonia in water, Mobile phase(B):ACN, Flow rate: 1.0 mL/min) to afford 7-(6,7-dimethoxy-3-methylquinolin-4-yl)-3,4-dihydroisoquinoline-2(1H)-sulfonamide (0.014 g, 12%) as off-white solid.¹H NMR (400 MHz, DMSO-d₆): δ 8.63(s, 1 H), 7.38(s, 1 H), 7.35(d, J=8.0 Hz, 1 H), 7.14-7.11(m, 2 H), 6.90(s, 2 H), 6.66(s, 1 H), 4.26(s, 2H), 3.90(s, 3H), 3.61(s, 3H), 3.34-3.31(m, 2H), 3.08-2.59 (m, 2H), 2.15 (s, 3 H); LC-MS (ES) m/z =414. 1[M+H]⁺; HPLC purity 99.78 %. at 254 nm.

Example 61: N-(4-(6,7-dimethoxyquinolin-4-yl)benzyl)-N-methylsulfamide:

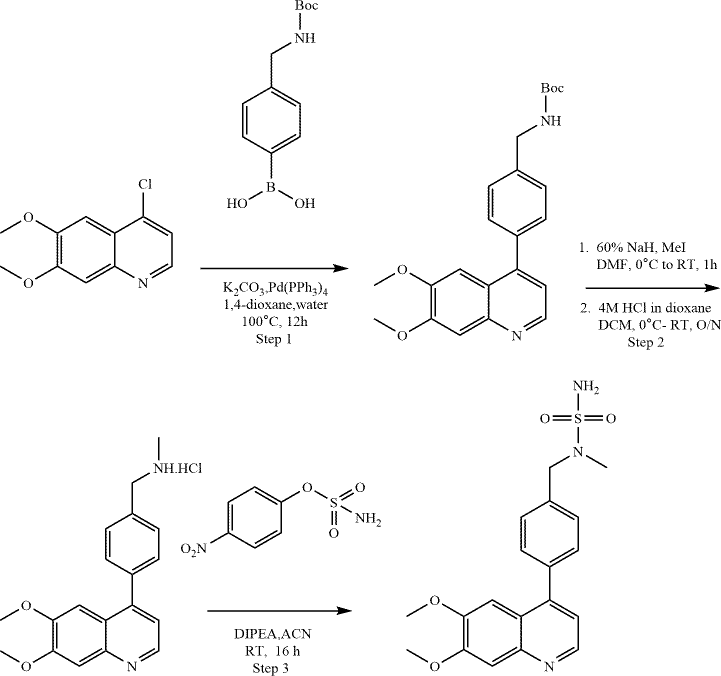

### Step 1: Tert-Butyl (4-(6,7-Dimethoxyquinolin-4-yl)Benzyl)Carbamate

Procedure: To a stirred solution of 4-chloro-6,7-dimethoxyquinoline (0.5 g, 2.242 mmol), (4-((tert-butoxycarbonyl)amino)methyl)phenyl)boronic acid (0.56 g, 2.242 mmol) and potassium carbonate (0.92 g, 6.726 mmol) in 1,4-dioxane ( 10 mL) and water ( 3 mL) was degassed with nitrogen gas for 10 min, then tetrakis(triphenylphosphine)palladium(0) (0.13 g, 0.1121 mmol) was added and heated for 12 h at 100° C. The reaction mixture was cooled to room temperature, filtered through celite bed, the filtrate was dried and concentrated to give crude product. The crude was purified by flash column with silica gel pack and 30% ethyl acetate in hexane as eluent. The pure fractions were collected and concentrated to afford tert-butyl (4-(6,7-dimethoxyquinolin-4-yl)benzyl)carbamate as off white solid (0.62 g, 70%).¹HNMR (400 MHz, DMSO-d₆): δ 8.69 (d, J= 3.6 Hz, 1 H), 7.65 - 7.55 (m, 1 H), 7.55 - 7.50 (m, 2 H), 7.50 - 7.40 (m, 3 H), 7.23 (d, J= 3.6 Hz, 1 H), 7.15 (s, 1 H), 4.23 (d, J= 4.8 Hz, 2 H), 3.94 (s, 3 H), 3.73 (s, 3 H), 1.40 (s, 9 H). LC-MS (ES) m/z = 395.1 [M+H]+ .

### Step 2: 1-(4-(6,7-Dimethoxyquinolin-4-yl)Phenyl)-N-Methylmethanamine Hydrochloride

Procedure: To a stirred solution of tert-butyl (4-(6,7-dimethoxyquinolin-4-yl)benzyl)carbamate (0.42 g, 1.065 mmol) in DMF ( 10 mL) was added 60% sodium hydride (0.051 g, 1.279 mmol) at 0° C. and stirred for 15 min. Methyl iodide (0.1 mL, 1.598 mmol) was added at 0° C. The reaction mixture was stirred for 30 min, and then quenched with ice water, extracted with ethyl acetate. The organic layer was dried over sodium sulfate, filtered and concentrated to afford tert-butyl (4-(6,7-dimethoxyquinolin-4-yl)benzyl)(methyl) carbamate as brown color viscous liquid (0.4 g, 91.97%). LC-MS (ES) m/z = 409.1 [M+H]⁺.

To a stirred solution of the carbamate (0.4 g, 0.980 mmol) in DCM (10 mL) was added 4M HC1 in 1,4-dioxane (20 mL) at 0° C. and stirred for overnight at room temperature. The reaction mixture was concentrated to afford 1-(4-(6,7-dimethoxyquinolin-4-yl)phenyl)-N-methylmethanamine hydrochloride (0.4 g crude). LC-MS (ES) m/z = 309.1 [M+H]⁺.

### Step 3: N-(4-(6,7-Dimethoxyquinolin-4-yl)Benzyl)-N-Methylsulfamide

Procedure: To a stirred solution of compound 1-(4-(6,7-dimethoxyquinolin-4-yl)phenyl)-N-methylmethanamine hydrochloride (0.4 g, 1.1627 mmol) in acetonitrile (10 mL) was added DIPEA (0.62 mL, 3.488 mmol) and stirred for 5 min. 4-nitrophenyl sulfamate (0.33 g, 1.511 mmol) was added at room temperature, stirred for overnight at room temperature. The reaction mixture was concentrated. The residue was diluted with water and ethyl acetate. The organic layer was separated and dried over sodium sulfate, filtered and concentrated to give crude product. The crude product was purified by flash column with silica gel pack and 3% methanol in DCM as eluent. The pure fractions were collected and concentrated to afford N-(4-(6,7-dimethoxyquinolin-4-yl)benzyl)-N-methylsulfamide as off white solid (95 mg, 21%). ¹H NMR (400 MHz, DMSO-d₆): δ 8.69 (d, J= 4.8 Hz, 1 H), 7.58 (d, J= 8.4 Hz, 2 H), 7.52 (d, J= 7.6 Hz, 2 H), 7.44 (s, 1H), 7.25 (d, J= 4.4 Hz, 1H), 7.16 (s, 1 H), 6.90 (s, 2 H), 4.18 (s, 2 H), 3.93 (s, 3 H), 3.74 (s, 3 H), 2.60 (s, 3 H), LC-MS (ES) m/z = 388.1 [M+H]⁺ , HPLC purity 99.87% at 254 nm.

Example 62: N-(2-fluoro-4-(7-methoxyquinolin-4-yl)benzyl)sulfamide:

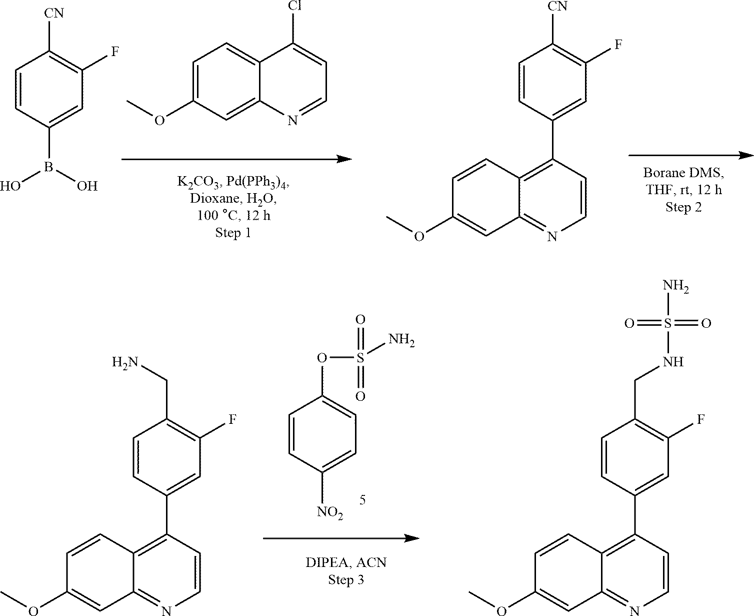

### Step 1 2-Fluoro-4-(7-Methoxyquinolin-4-yl)Benzonitrile

Procedure: To a stirred solution of 4-chloro-7-methoxyquinoline (0.6 g, 3.108 mmol) in dioxane (20 mL) and water (5 mL) was added (4-cyano-3-fluorophenyl)boronic acid (0.51 g, 3.108 mmol) and potassium carbonate (1.25 g, 9.324 mmol), degassed the resulting mixture with nitrogen gas for 10 min. Tetrakis(triphenyl phosphine) palladium (0.18 g, 0.155 mmol) was added and heated at 100° C. for overnight in a sealed tube. The progress of the reaction was monitored by TLC. The reaction mixture was cooled to room temperature, diluted with water and ethyl acetate. Organic layer was separated and dried over sodium sulfate. The solvent was evaporated to give crude product, which was purified by flash chromatography using 40 % ethyl acetate in hexane to afford 2-fluoro-4-(7-methoxyquinolin-4-yl)benzonitrile as yellow solid (0.6 g, 70%). ¹H NMR (400 MHz, CDC13)— δ 8.89 (d, J= 4.4 Hz, 1 H), 7.79 (t, J= 7.2 Hz, 7.6 Hz, 1 H), 7.64 (d, J= 9.2 Hz, 1 H), 7.53 (s, 1 H), 7.41 - 7.36 (m, 2 H), 7.22 - 7.16 (m, 2 H), 3.98 (s, 3 H). LC-MS (ES) m/z = 279.0[M+H]⁺.

### Step 2: (2-Fluoro-4-(7-Methoxyquinolin-4-yl)Phenyl)Methanamine

Procedure: To a stirred solution of 2-fluoro-4-(7-methoxyquinolin-4-yl)benzonitrile (0.3 g, 1.079 mmol) in tetrahydrofuran (30 mL) was added borane dimethylsulfide (0.404 g, 0.5 mL, 5.395 mmol ) slowly drop wise at 0° C. over a period of 10 min. The reaction mixture was stirred overnight at room temperature. The progress of the reaction was monitored by TLC. The reaction mixture was cooled to 0° C. and quenched with methanol slowly, then heated at 70° C. for 0.5 h. The reaction mixture was evaporated under reduce pressure to afford (2-fluoro-4-(7-methoxyquinolin-4-yl)phenyl)methanamine (0.3 g, crude) as black solid. LC-MS (ES) m/z = 283.1 [M+H]⁺.

### Step 3: N-(2-Fluoro-4-(7-Methoxyquinolin-4-yl)Benzyl)Sulfamide

Procedure: To a stirred solution of (2-fluoro-4-(7-methoxyquinolin-4-yl)phenyl) methanamine (0.3 g, 1.063 mmol) in acetonitrile (10 mL) was added DIPEA (0.55 mL, 3.191 mmol) and stirred for 5 min. 4-nitrophenyl sulfamate (0.2 g, 1.59 mmol) was added at room temperature, stirred for 24 h at room temperature. The progress of the reaction was monitored by TLC. The reaction mixture was evaporated under reduced pressure to give crude product, which was purified by flash chromatography using 5-10 % MeOH/DCM as eluent. Pure fractions were collected and evaporated to give impure product. It was again purified by preparative HPLC using Inertsil ODS 3V column, and 0.1 % ammonia in water in acetonitrile as mobile phase to afford N-(2-fluoro-4-(7-methoxyquinolin-4-yl)benzyl)sulfamide as off-white solid (0.015 g, 10 %). ¹H NMR (400 MHz, DMSO-d₆): δ 8.85 (d, J= 4.4 Hz, 1 H), 7.74 (d, J= 9.2 Hz, 1 H), 7.66 (t, J= 8.2 Hz, 1 H), 7.47 (s, 1 H), 7.37 - 7.23 (m, 4 H), 7.16 (bs, 1 H), 6.69 (bs, 2 H), 4.21 (s, 2 H), 3.92 (s, 3 H). LC-MS (ES) m/z = 362.0 [M+H] ^{+,} HPLC purity 99.74% at 254 nm.

Example 63: N-(4-(7-methoxy-1,8-naphthyridin-4-yl)benzyl)sulfamide:

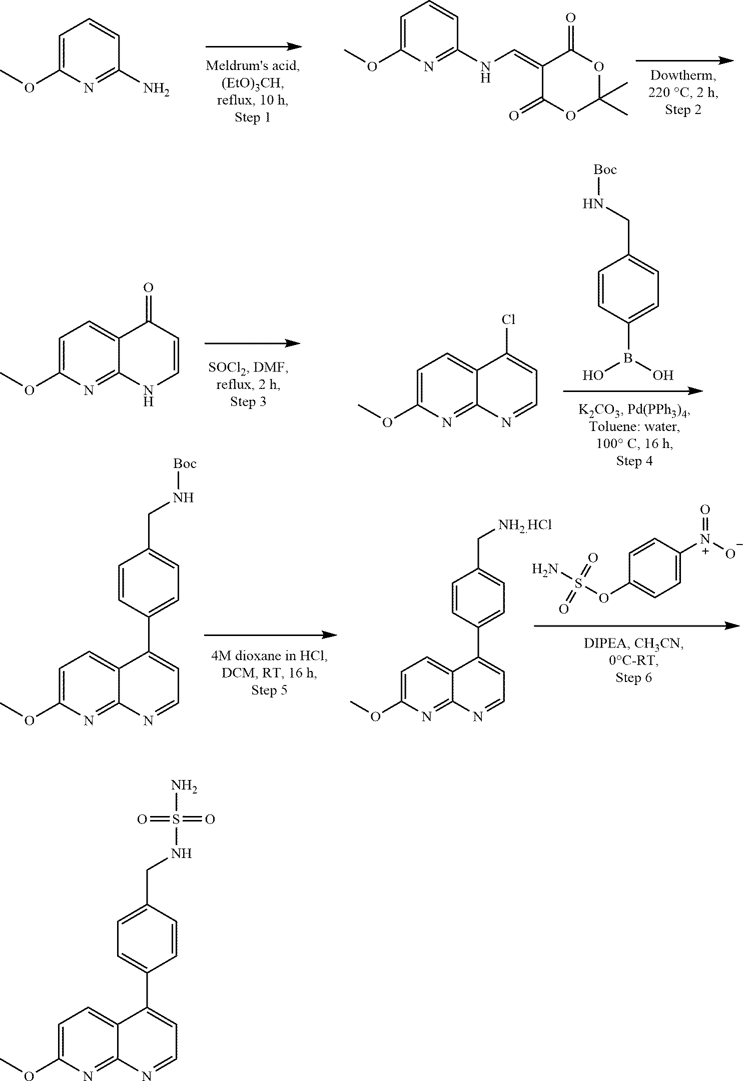

### Step 1: 5-(((6-Methoxypyridin-2-yl)Amino)Methylene)-2,2-Dimethyl-1,3-Dioxane-4,6-Dione

Procedure: A solution of Meldrum’s acid (3.49 g, 24 mmol) in triethyl orthoformate (40 mL) was heated at 105° C. for 2 h. 6-methoxypyridin-2-amine (3.0 g, 24.1 mmol) was added and the resulting reaction mixture was further heated at 105° C. for 10 h. The progress of the reaction was monitored by TLC using 30% ethyl acetate in hexane as eluent. The reaction mixture was cooled to room temperature. The solid formed in the reaction mixture was filtered and dried under vacuum pressure to afford 5-(((6-methoxypyridin-2-yl) amino) methylene)-2,2-dimethyl-1,3-dioxane-4,6-dione as brown solid (3.5 g, 52 %). ¹HNMR (400 MHz, DMSO-d₆): δ 11.30 (d, J= 10.0 Hz, 1 H), 9.16 (d, J= 11.6 Hz, 1 H), 7.76 (t, J= 8.0 Hz, 1 H), 7.18 (d, J= 7.6 Hz, 1 H), 6.68 (d, J= 8 Hz, 1 H), 3.89 (s, 3 H), 1.66 (s, 6 H). LC-MS (ES) m/z = 279 [M+H]⁺.

### Step 2: 7-Methoxy-1,8-Naphthyridin-4(1h)-One

Procedure: A solution of 5-(((6-methoxypyridin-2-yl)amino)methylene)-2,2-dimethyl-1,3-dioxane-4,6-dione (3.5 g, 12 mmol) in Dowtherm (30 mL) was heated at 220° C. for 4 h . The progress of the reaction was monitored by TLC. The reaction mixture was cooled to room temperature and the precipitated solid was filtered. The residue was thoroughly washed with n-hexanes and dried under vacuum to obtain 7-methoxy-1,8-naphthyridin-4(1H)-one (2.0 g, 92 %). ¹HNMR (400 MHz, DMSO-d₆): δ 11.91 (bs, 1 H), 8.27 (d, J= 8.8 Hz, 1 H), 7.74 - 7.71 (m, 1 H), 6.76 (d, J= 8.8 Hz, 1 H), 6.01 (d, J= 7.2 Hz, 1 H), 3.94 (s, 3 H). LC-MS (ES) m/z = 177.1 [M+H]⁺ .

### Step 3: 5-Chloro-2-Methoxy-1,8-Naphthyridine

Procedure: To a suspension of 7-methoxy-1,8-naphthyridin-4(1H)-one (0.4 g, 2.2 mmol) in thionyl chloride (15 mL) was added a drop of DMF and the mixture was heated to reflux for 2 h. The progress of the reaction was monitored by TLC. The mixture was concentrated under reduced pressure. The residue was basified by saturated sodium bicarbonate solution and extracted with ethyl acetate. The organic layer was dried over sodium sulphate and evaporated to obtain 5-chloro-2-methoxy-1,8-naphthyridine as brown solid (0.92 g, 81.8 %). LC-MS (ES) m/z = 195.0 [M+H]⁺.

### Step 4: Tert-Butyl(4-(7-Methoxy-1,8-Naphthyridin-4-yl)Benzyl)Carbamate

Procedure: To a stirred solution of 4-chloro-6,7-dimethoxyquinazoline (0.3 g, 1.5 mmol) in toluene (15 mL) and water (5 mL) was added potassium carbonate (0.635 g, 4.6 mmol) and degassed with argon for 10 min. (4-(((tert-butoxycarbonyl)amino)methyl)phenyl)boronic acid (0.770 g, 3.01 mmol) and tetrakis(triphenylphosphine) palladium(0) (0.09 g, 0.07 mmol) was added to above solution and heated at 100° C. for 16 h. The progress of the reaction was monitored by TLC. Reaction mixture was diluted with water, extracted using ethyl acetate (3 x 20 mL), organic layer was dried over sodium sulphate and evaporated to obtain crude product, which was purified by silica gel flash column chromatography. The compound eluted out in 50 % EtOAc/hexanes. Pure fractions were evaporated to obtain tert-butyl(4-(7-methoxy-1,8-naphthyridin-4-yl)benzyl)carbamate as white solid (0.350 g, 62.01 %). ¹HNMR (400 MHz, DMSO-d₆): δ 8.91 (d, J= 4.4 Hz 1H), 8.12 (d, J= 9.2 Hz, 1 H), 7.59 -7.52 (m, 1 H), 7.49-7.38 (m, 5 H), 7.08 (d, J= 8.4 Hz, 1 H), 4.22 (d, J= 6.4 Hz, 2 H), 4.0 (s, 3 H), 1.39 (s, 9 H). LC-MS (ES) m/z = 366.1 [M+H]⁺.

### Step 4: (4-(7-Methoxy-1,8-Naphthyridin-4-yl)Phenyl)Methanamine Hydrochloride

Procedure: To a stirred solution of tert-butyl(4-(7-methoxy-1,8-naphthyridin-4-yl)benzyl) carbamate (0.350 g, 0.95 mmol) in dichloromethane (15 mL) was added HC1 in 1,4-Dioxane (3.5 mL, 4M solution) at 0° C. The reaction mixture was gradually warmed to room temperature and stirred for 16 h. The progress of the reaction was monitored by TLC. Reaction mixture was concentrated under reduced pressure to afford (4-(7-methoxy-1,8-naphthyridin-4-yl)phenyl)methanamine hydrochloride as white solid (0.25 g, 80%). ¹HNMR (400 MHz, DMSO-d₆): δ 9.05 (d, J= 4.8 Hz 1H), 8.53 (bs, 3H), 8.18 (d, J= 9.2 Hz, 1H), 7.73 (d, J= 8 Hz, 2H), 7.64 (d, J = 7.2 Hz, 2H), 7.28 (d, J = 9.2 Hz, 1H), 4.14 (d, J = 6 Hz, 2H), 4.09 (s, 3H).LC-MS (ES) m/z = 266.1 [M+H]⁺.

### Step 5: N-(4-(7-Methoxy-1,8-Naphthyridin-4-yl)Benzyl)Sulfamide

Procedure: To a stirred solution of (4-(7-methoxy-1,8-naphthyridin-4-yl)phenyl)methanamine (0.250 g, 0.94 mmol) and 4-nitrophenyl sulfamate (0.407 g, 1.84 mmol) in acetonitrile (10 mL) was added N,N-diisopropylethylamine (0.23 mL, 1.41 mmol) at 0° C. and the mixture was stirred at room temperature for 16 h. The progress of the reaction was monitored by TLC. The solvent was evaporated, and the crude was purified by silica gel flash column chromatography. The compound eluted out in 4-5 % MeOH/DCM. Fractions containing pure compound were evaporated to obtain N-(4-(7-methoxy-1,8-naphthyridin-4-yl)benzyl) sulfamide as off-white solid (0.049 g, 14 %) ¹HNMR (400 MHz, DMSO-d₆): δ 8.92 (d, J= 4 Hz, 1H), 8.12 (d, J= 9.2 Hz, 1H), 7.54 (d, J= 8 Hz, 2 H), 7.48 (d, J= 8 Hz, 2 H), 7.39 (d, J = 4.8 Hz, 1 H), 7.15-7.08 (m, 2 H), 6.64 (s, 2 H), 4.18 (d, J= 6.4 Hz, 2 H), 4.03 (s, 3 H). LC-MS (ES) m/z =345.0 [M+H]⁺. HPLC purity 99.03 % at 254 nm.

Examples 64-80: Examples 64-80 were synthesized as described for examples 1-63.

| Ex. | LCMS [M+H] + | ¹H-NMR (400 MHz, DMSO-d₆): δ |
|---|---|---|
| 64 | 432.4 | 8.58 (s, 1 H), 7.35 (s, 1 H), 7.22 (s, 1 H), 6.75 (s, 2 H), 3.94 (s, 6 H), 3.63 - 3.52 (m, 4 H), 3.24 (t, J= 7.6 Hz, 2 H), 3.12 (s, 2 H), 1.87 - 1.76 (m, 6 H); HPLC purity: 99.93% at 254 nm. |
| 65 | 378.4 | 8.52 (s, 1 H), 7.85 (d, J= 8.8 Hz, 1 H), 7.16 - 7.10 (m, 2 H), 6.72 (s, 2 H), 3.89 (s, 3 H), 3.70 - 3.60 (m, 4 H), 3.22 (t, J= 6.8 Hz, 2H), 3.05 (s, 2 H), 1.79 (t, J= 7.2 Hz, 2 H), 1.75 - 1.61 (m, 4 H); HPLC purity 99.89% at 254 nm. |
| 66 | 413.3 | 8.49 (d, J= 4.4 Hz, 1 H), 7.3 (s, 1 H), 7.22 (s, 1 H), 6.44 (bs, 3 H), 3.9 (s, 3H), 3.88 (s, 3 H), 3.37-3.34 (m, 2 H), 3.12 (t, J= 8.4 Hz, 2 H), 2.96 (q, J= 6.4 Hz, 2 H), 1.81-1.78 (m, 2 H), 1.53-1.31 (m, 5 H), HPLC purity: 98.21% at 254 nm. |
| 67 | 392.3 | 8.76 (s, 1 H), 7.57-7.55 (m, 2 H), 7.49-7.46 (m, 3 H), 7.17 (bs, 1 H), 6.89 (s, 1 H), 6.67 (s, 2H), 4.19 (d, J = 6 Hz, 2 H), 3.92 (s, 3 H), 3.69 (s, 3 H), HPLC Purity: 99.35 % at 254 nm. |
| 68 | 415.0 | 8.70 (d, J = 4.4 Hz, 1 H), 7.52 (bs, 4 H), 7.44 (s, 1 H), 7.26 (d, J = 4.4 Hz, 1 H), 7.13 (s, 1 H), 6.98 (bs, 2 H), 4.36 (s, 2 H), 3.94 (s, 3 H), 3.73 (s, 3 H), 2.39 (m, 1 H), 0.57 (m, 4 H). HPLC purity 99.1 %. at 254 nm. |
| 69 | 391.95 | 8.70 (d, J = 4.8 Hz, 1 H), 7.66 (t, J = 8.4 Hz, 1 H), 7.46 - 7.38 (m, 3 H), 7.25 (d, J = 4.4 Hz, 1 H), 7.18 - 7.12 (m, 2 H), 6.69 (s, 2 H), 4.23 (d, J = 6.8 Hz, 2 H),3.94 (s, 3 H) 3.75 (s, 3 H). HPLC purity 98.86 % at 254 nm |
| 70 | 378.1 | 8.53(s, 1 H), 7.74(d, J =9.2 Hz, 1 H), 7.47-7.45(m, 1 H), 7.17(s, 1 H), 6.72(s, 2H), 3.88(s, 3 H), 3.68-3.63(m, 4 H), 3.21(t, J =7.2 Hz, 2 H), 3.06(s, 2 H), 1.82-1.72(m, 6 H). HPLC purity 99.75 % at 254 nm. |
| 71 | 364.0 | 9.88 (bs, 1 H), 8.59 (d, J=5.2 Hz, 1 H), 8.20 (d, J=8.8 Hz, 1 H), 7.41 - 7.37 (m, 2 H), 7.28 (bs, 3 H), 7.21(d, J=12.8 Hz, 1 H), 7.03 (d, J= 8.8 Hz, 1 H), 6.41 (d, J= 4.8 Hz, 1 H), 3.92 (s, 3 H); HPLC purity: 99.54 % at 254 nm. |
| 72 | 389.0 | 8.99 (s, 1 H), 8.65 (d, J = 4.8 Hz, 1 H), 7.59 - 7.54 (m, 2 H), 7.19 (d, J = 5.2 Hz, 1 H), 7.14(s, 1 H), 6.85 - 6.80 (m, 3 H), 6.40 (d, J = 8.4 Hz, 1 H), 4.04 -3.99 (m, 5 H), 3.14 - 3.10 (m, 2 H). HPLC purity 99.16 % at 254 nm. |
| 73 | 346.4 | 9.58 (s, 1 H), 8.66 (d, J = 5.6 Hz, 1 H), 8.57 (d, J = 8.8 Hz, 1 H), 7.28 - 7.20 (m, 4 H), 7.13 - 7.11 (m, 3 H), 6.54 (d, J = 5.6 Hz, 1 H), 3.92 (s, 3 H). HPLC purity 99.51 % at 254 nm. |
| 74 | 349.0 | 8.67 (d, J = 4.8 Hz, 1 H), 7.92 - 7.91 (m, 3H), 7.63 (d, J = 8 Hz, 1 H), 7.42 -7.40 (m, 4 H), 7.41 (d, J = 4.8 Hz, 1 H), 3.95 (s, 3 H). HPLC purity: 99.11 % at 254 nm. |
| 75 | 380.3 | 8.82 (s, 1 H), 8.62 (d, J = 5.2 Hz, 1 H), 8.14 (d, J = 8.8 Hz, 1 H), 7.62 (d, J = 8.8 Hz, 1 H), 7.47 (s, 1 H), 7.40 (s, 1 H),7.28 - 7.26 (m, 2 H),7.15 (s, 2 H), 6.53 (d, J = 4.8 Hz, 1 H), 3.91(s, 3 H). HPLC purity 94.25 % at 254 nm. |
| 76 | 374.0 | 9.12 (s, 1 H), 7.84 - 7.78 (m, 4 H), 7.36 (s, 2 H), 7.26 (d, J = 8.8 Hz, 2 H), 4.07 (s, 3 H). HPLC purity: 98.45 % at 254 nm. |
| 77 | 359.9 | 8.61 (d, J = 4.8 Hz, 1 H), 8.15 (d, J = 8.4 Hz, 1 H), 7.45 (d, J = 8 Hz, 2 H), 7.39 (d, J = 2 Hz, 1 H), 7.28 - 7.24 (m, 3 H), 7.026 (bs, 2 H), 6.51(d, J = 5.2 Hz, 1H), 3.92 (s, 3 H), 3.12 (s, 3 H). HPLC purity: 99.15 % at 254 nm. |
| 78 | 375.9 | 8.59 (d, J = 4.8 Hz, 1 H), 8.18 (d, J = 9.2 Hz, 1 H), 8.08 (br s, 1 H), 7.44 (d, J = 8.8 Hz, 1 H), 7.38 (s, 1 H), 7.26 (dd, J = 9.2, 2.4 Hz, 1 H), 7.00 - 6.99 (m, 3H), 6.80 (dd, J = 8.4, 2.0 Hz, 1 H), 6.49 (d, J = 4.8 Hz, 1 H), 3.92 (s, 3 H), 3.78 (s, 3 H); HPLC purity: 99.09% at 254 nm. |
| 79 | 382.3 | 9.58 (s, 1 H), 8.62 (d, J=5.2 1 H), 8.18 (d, J = 9.2 Hz, 1 H), 7.58-7.49 (m, 2H),7.41 (s, 1 H), 7.30-7.29 (m, 3 H), 6.53 (d, J = 4.8 Hz, 1 H), 3.92(s, 3 H), HPLC purity=99.87% at 254 nm. |
| 80 | 356.0 | 9.13 (s, 1 H), 7.90 (d, J = 9.2 Hz, 1 H), 7.82 (d, J = 8.8 Hz, 2 H), 7.58 (s, 1H), 7.39 - 7.35 (m, 3 H), 7.24 (d, J = 8.4 Hz, 2 H), 3.98 (s, 3 H); HPLC purity: 99.9% at 254 nm. |

Example 81: N-(4-((6,7-dimethoxyquinazolin-4-yl)oxy)benzyl)cyclopropanesulfonamide:

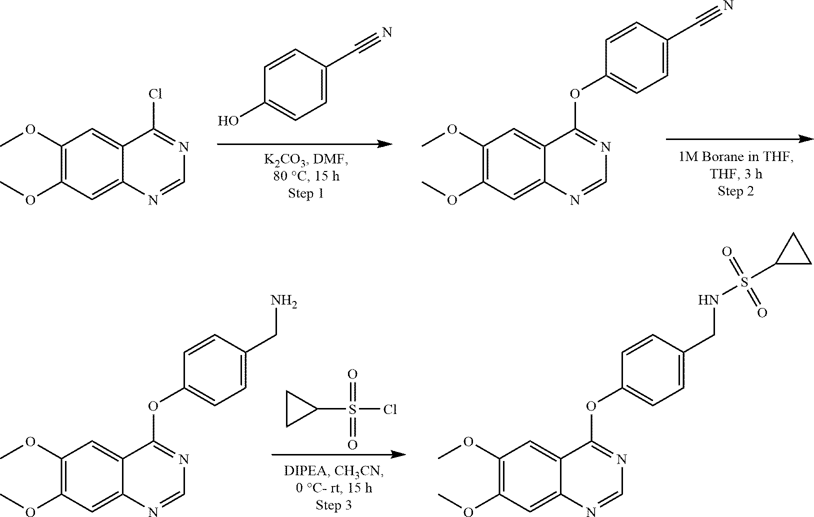

### Step 1: 4-((6,7-Dimethoxyquinazolin-4-yl)Oxy)Benzonitrile

Procedure: To a stirred solution of 4-chloro-6,7-dimethoxyquinazoline (0.5 g, 2.2257 mmol) in DMF (5 mL) was added 4-hydroxybenzonitrile (0.29 g, 2.44 mmol) followed by K₂CO₃ (0.92 g, 6.677 mmol) and heated at 80° C. for 15 h. The progress of the reaction was monitored by TLC. The reaction mixture was quenched with ice-water and filtered. The solid was washed with water followed by hexane to afford 4-((6,7-dimethoxyquinazolin-4-yl)oxy) benzonitrile as off-white solid (0.68 g, 88%). ¹HNMR (400 MHz, DMSO-d₆): δ 8.57(s, 1 H), 7.97(d, J=8.4 Hz, 2 H), 7.58-7.55 (m, 3 H), 7.40(s, 1 H), 3.98(s, 3 H), 3.96(s, 3 H); LC-MS (ES) m/z =308.0 [M+H]⁺.

### Step 2: (4-((6,7-Dimethoxyquinazolin-4-yl)Oxy)Phenyl)Methanamine

Procedure: To a stirred solution of 4-((6,7-dimethoxyquinazolin-4-yl)oxy)benzonitrile (0.2 g, 0.650 mmol) in THF (5 mL) was added 1M borane in THF (1.95 mL, 1.952 mmol). The reaction mixture was allowed to stir at 60° C. for 3 h. The progress of the reaction was monitored by TLC. Then the reaction was quenched with 1.25 M HCl and again refluxed for 2 h. The mixture was cooled to room temperature, solid precipitated was filtered and washed with diethyl ether and dried to give (4-((6,7-dimethoxyquinazolin-4-yl)oxy)phenyl)methanamine as yellow solid (0.08 g, Crude). LC-MS (ES) m/z = 312.1[M+H] ⁺.

### Step 3: N-(4-((6,7-Dimethoxyquinazolin-4-yl)Oxy)Benzyl)Cyclopropanesulfonamide

Procedure: To a stirred solution of (4-((6,7-dimethoxyquinazolin-4-yl)oxy)phenyl)methanamine (0.08 g, 0.256 mmol) in acetonitrile (5 mL) was added cyclopropane sulfonyl chloride (0.04 mL, 0.333 mmol) followed by N,N-diisopropylethylamine (0.13 mL, 0.770 mmol) at 0° C. The reaction mixture was then allowed to stir at room temperature for 15 h. The progress of the reaction was monitored by TLC. The solvent was evaporated to give crude product. The crude residue was purified by gradient column chromatography using 1-6 % methanol in dichloromethane to give N-(4-((6,7-dimethoxyquinazolin-4-yl)oxy)benzyl)cyclopropanesulfonamide as off-white solid (0.015 g, 14%). ¹HNMR (400 MHz, DMSO-d₆): δ 8.51(s, 1 H), 7.70-7061(m, 1 H), 7.54(s, 1 H), 7.44(d, J =8.4 Hz, 2 H), 7.37(s, 1 H), 7.27(d, J=8.8 Hz, 2 H), 4.23(d, J=6.4 Hz, 2 H), 3.97(s, 3 H), 3.96(s, 3 H), 2.48(m, 1 H), 0.91-0.90(m, 4 H); LC-MS (ES) m/z =416.3[M+H]⁺; HPLC purity 98.81%.

Example 82: N-(4-(1-((6,7-dimethoxyquinazolin-4-yl)amino) ethyl)phenyl) cyclopropane sulfonamide:

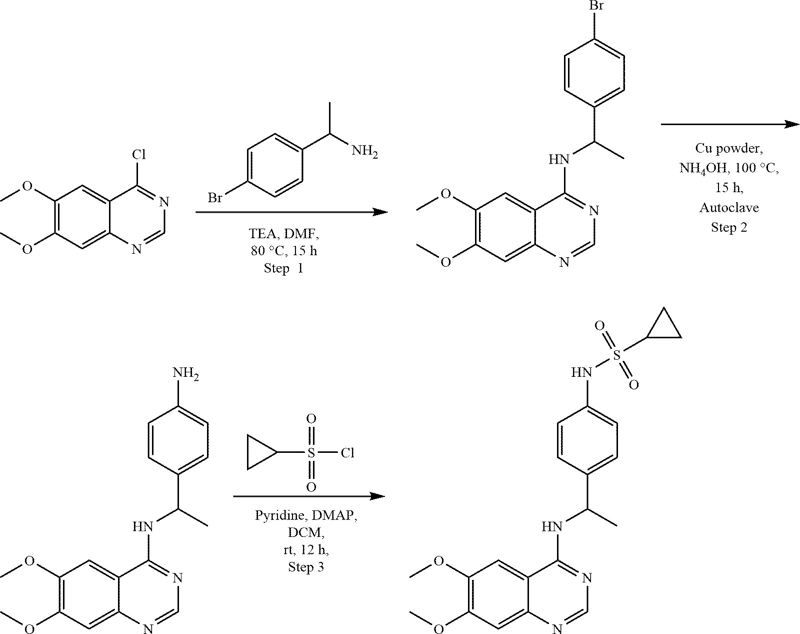

### Step 1: N-(1-(4-Bromophenyl)Ethyl)-6,7-Dimethoxyquinazolin-4-Amine

Procedure: To a stirred solution of 4-chloro-6,7-dimethoxyquinazoline (0.5 g, 2.231 mmol) in DMF (5 mL) was added 1-(4-bromophenyl)ethan-1-amine (0.53 g, 2.67 mmol) followed by triethylamine (0.93 g, 6.695 mmol) and heated at 80° C. for 15 h. The progress of the reaction was monitored by TLC. The reaction mixture was quenched with ice-water and filtered. The solid was washed with water, followed by pentane to afford N-(1-(4-bromophenyl)ethyl)-6,7-dimethoxyquinazolin-4-amine as an off-white solid (0.65 g, 75%). ¹HNMR (400 MHz, DMSO-d₆): δ 8.24(s, 1 H), 8.05(d, J =7.6 Hz, 1 H), 7.72 (s, 1 H), 7.48(d, J=8.4 Hz, 2 H), 7.36(d, J=8.4 Hz, 2 H), 7.06(s, 1 H), 5.54-5.50(m, 1 H), 3.91(s, 3 H), 3.87(s, 3 H), 1.55(t, J=6.8 Hz, 3 H); LC-MS (ES) m/z =389.0 [M+2H]⁺.

### Step 2: N-(1-(4-Aminophenyl)Ethyl)-6,7-Dimethoxyquinazolin-4-Amine

Procedure: To a stirred solution of N-(1-(4-bromophenyl)ethyl)-6,7-dimethoxyquinazolin-4-amine (0.5 g, 1.28 mmol) in ammonium hydroxide (50 mL) in an autoclave was added Cu powder (0.04 g, 0.064 mmol) and heated at 100° C. for 15 h. The progress of the reaction was monitored by TLC. The reaction mixture was cooled to room temperature, diluted with 10% methanol in dichloromethane and filtered through celite bed. The filtrate was collected and the organic layer was separated and dried over anhydrous Na₂SO₄. The filtrate obtained was evaporated under reduced pressure to give N-(1-(4-aminophenyl) ethyl)-6,7-dimethoxyquinazolin-4-amine as an off-white solid (0.4 g, crude). LC-MS (ES) m/z =325.1 [M+H]⁺.

### Step 3: N-(4-(1-((6,7-Dimethoxyquinazolin-4-yl)Amino)Ethyl)Phenyl)Cyclopropanesulfonamide

Procedure: To a stirred solution of N-(1-(4-aminophenyl)ethyl)-6,7-dimethoxyquinazolin-4-amine (0.1 g, 0.308 mmol) in DCM (10 mL) was added pyridine (0.07 mL, 0.924 mmol) and DMAP (0.01 g, 0.154 mmol) followed by cyclopropanesulfonyl chloride (0.05 g, 0.369 mmol) and stirred at room temperature for 15 h. The progress of the reaction was monitored by TLC. The reaction mixture was diluted with 10% methanol in dichloromethane and washed with water. The organic layer was dried over sodium sulphate and concentrated. The crude product was purified by gradient column chromatography using 4-10% methanol in dichloromethane to afford N-(4-(1-((6,7-dimethoxyquinazolin-4-yl)amino)ethyl)phenyl)cyclopropanesulfonamide (0.015 g, 11%) as off-white solid.¹HNMR (400 MHz, DMSO-d₆): δ 9.57(s, 1 H), 8.25(s, 1 H), 8.01(d, J=6.4 Hz, 1 H), 7.72(s, 1 H), 7.35(d, J=8.4 Hz, 2 H), 7.15(d, J=8.4 Hz, 2 H), 7.06(s, 1 H), 5.58-5.55(m, 1 H), 3.90(s, 3 H), 3.87(s, 3 H), 2.50-2.48(m, 1 H), 1.55(d, J=6.8 Hz, 3 H), 0.89-0.88(m, 4 H);LC-MS (ES) m/z =429.3[M+H]⁺; HPLC purity 99.77%.

Example 83: N-(4-(3-cyano-6-methoxy-1,7-naphthyridin-4-yl)benzyl) cyclopropane sulfonamide:

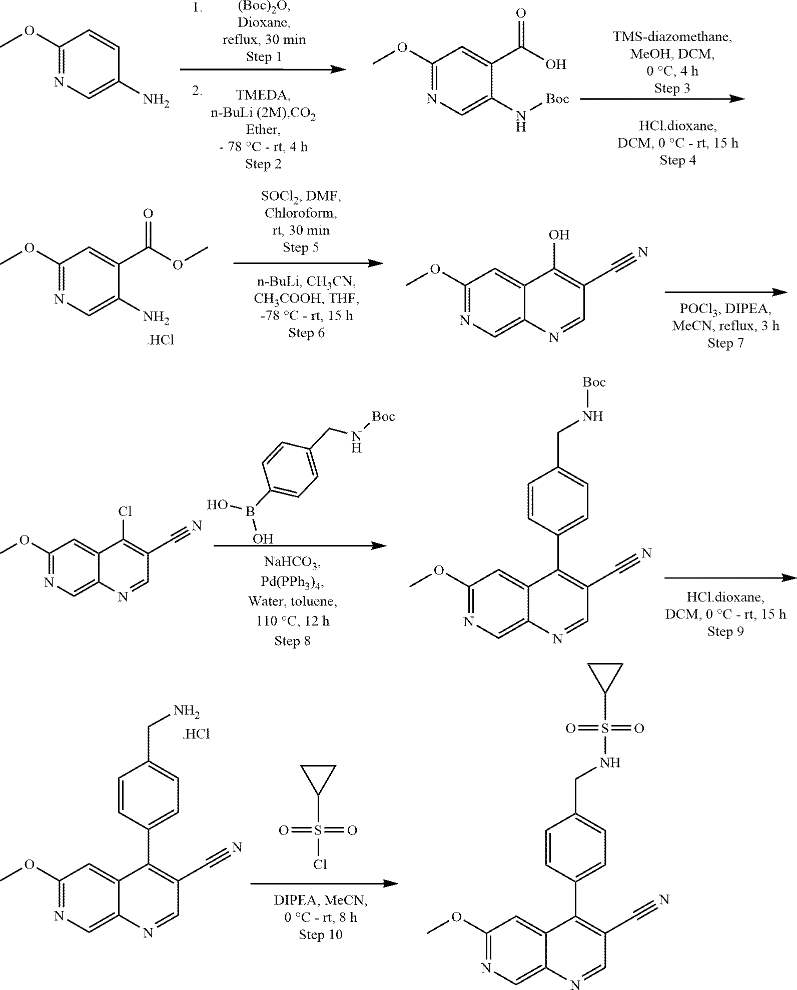

### Step 1: Tert-Butyl (6-Methoxypyridin-3-yl)Carbamate

Procedure: To a stirred solution of 6-methoxypyridin-3-amine (10 g, 80.554 mmol) in 1,4-dioxane (110 mL) was added di-tert-butyl dicarbonate (19.33 g, 88.609 mmol) and solution was heated to reflux for 30 min. The progress of the reaction was monitored by TLC. Reaction mixture was poured onto ice cold water, extracted using ethyl acetate (3 x 40 mL), and dried over sodium sulphate, filtered and concentrated to give crude product. The crude product was purified by gradient column chromatography using ethyl acetate in n-hexane to afford tert-butyl (6-methoxypyridin-3-yl)carbamate as off white solid (14.0 g, 77.49 %). ¹HNMR (400 MHz, CDC13): δ 8.00 (d, J = 2.8 Hz, 1 H), 7.80 (bs, 1 H), 6.70 (d, J = 8.8 Hz, 1 H), 6.35 (m, 1 H), 3.90 (s, 3 H), 1.50 (s, 9 H). LC-MS (ES) m/z = 225.0 [M+H]⁺.

### Step 2: 5-((Tert-Butoxycarbonyl)Amino)-2-Methoxyisonicotinic Acid

Procedure: To a stirred solution of tert-butyl (6-methoxypyridin-3-yl)carbamate (7 g, 31.231 mmol) and tetramethylethylenediamine (14.05 mL , 93.640 mmol) in ether (140 mL) was added n-BuLi (46.82 mL, 3 eq, 2M solution in cyclohexane) at -78° C. and the mixture was stirred at -10° C. for 3 h. After re-cooling to - 78° C., dry carbon dioxide gas was bubbled and stirred for 5 min. The resulting suspension was allowed to room temperature and diluted with water. Organic layer separated and washed with dil. ammonium hydroxide solution. The combined aq. layer was acidified to pH 6 with dil. HCl. The resulting precipitate was filtered, dried under vacuum to afford 5-((tert-butoxycarbonyl)amino)-2-methoxyisonicotinic acid as off white solid (7.2 g, 85.99 %). ¹HNMR (400 MHz, DMSO-d₆): δ 9.37 (bs, 1 H), 8.67 (s, 1 H), 7.11 (s, 1 H), 3.84 (s, 3 H), 1.44 (s, 9 H). LC-MS (ES) m/z = 269.1 [M+H]⁺.

### Step 3: Methyl 5-((Tert-Butoxycarbonyl)Amino)-2-Methoxyisonicotinate

Procedure: To a stirred solution of 5-((tert-butoxycarbonyl)amino)-2-methoxyisonicotinic acid (6.7 g, 24.974 mmol) in methanol / dichloromethane (6.7 mL / 67 mL) was added TMS-diazomethane (31.2 mL, 2.5 eq) at 0° C. and the mixture was stirred at same temperature for 4 h. Reaction progress was monitored by TLC. Reaction mixture was concentrated, and residue was diluted with water, extracted using ethyl acetate (40 mL x 3), organic layer was dried over sodium sulphate, filtered and concentrated to give crude product. The crude product was purified by gradient column chromatography using ethyl acetate in n-hexane to afford methyl 5-((tert-butoxycarbonyl) amino)-2-methoxyisonicotinate as off-white solid (4.5 g, 63.82 %). ¹HNMR (400 MHz, CDC13): δ 9.24 (bs, 1 H), 9.18 (s, 1 H), 7.23 (s, 1 H), 3.93 (s, 3 H), 3.92 (s, 3 H), 1.52 (s, 9 H). LC-MS (ES) m/z = 283.1.

### Step 4: Methyl 5-Amino-2-Methoxyisonicotinate Hydrochloride

Procedure: To a stirred solution methyl 5-((tert-butoxycarbonyl)amino)-2-methoxyisonicotinate (3.7 g,13.106 mmol) in dichloromethane (40 mL ) was added HCl. dioxane (32.76 mL, 4 M solution) at 0° C. and the mixture was stirred at room temperature for 15 h. The progress of the reaction was monitored by TLC. Reaction mixture was concentrated and solid filtered, washed with n-hexane, dried under vacuum to afford methyl 5-amino-2-methoxyisonicotinate hydrochloride as yellow solid (2.8 g, 97.73 %). LC-MS (ES) m/z = 183.1 [M+H]⁺.

### Step 5: Methyl (E)-5-(((Dimethylamino)Methylene)Amino)-2-Methoxyisonicotinate

Procedure: To a stirred solution of methyl 5-amino-2-methoxyisonicotinate hydrochloride (2.8 g, 12.806 mmol) and dimethyl formamide (11.2 mL, 4 volume) in chloroform (56 mL, 20 volume) was added thionyl chloride (10.3 mL, 3.7 volume) drop wise at room temperature. The reaction mixture was stirred at room temperature for 30 min. Reaction progress was monitored by TLC. Reaction mixture was poured onto ice cold water and aq. layer was neutralized with saturated sodium bicarbonate solution. Reaction mixture was extracted with chloroform (30 mL X 3), dried over sodium sulphate and concentrated to give crude product which is used in next step without further purification (3 g, crude). LC-MS (ES) m/z = 238.1.

### Step 6: 4-Hydroxy-6-Methoxy-1,7-Naphthyridine-3-Carbonitrile

Procedure: To a stirred solution of n-butyl lithium (18.9 mL, 3 eq, 2M in cyclohexane) in tetrahydrofuran (20 mL) was added acetonitrile (2.66 mL, 50.572 mmol) in tetrahydrofuran (10 mL) drop wise at -78° C. under argon atmosphere. The resulting reaction mixture was stirred at -78° C. for 30 min. To the resulting white suspension was added a solution of methyl (E)-5-(((dimethylamino)methylene)amino)-2-methoxyisonicotinate (3 g, 12.644 mmol, in 20 mL tetrahydrofuran) at -78° C. and reaction mixture stirred at room temperature for 12 h . The reaction mixture was cooled to - 78° C. and glacial acetic acid (3.63 mL, 63.213 mmol) was added to above solution and resulting mixture was stirred at room temperature for 2 h. The progress of the reaction was monitored by TLC. Reaction mixture was diluted with water and solid was filtered. The crude product was purified by gradient column chromatography using methanol in dichloromethane to afford 4-hydroxy-6-methoxy-1,7-naphthyridine-3-carbonitrile as black solid (0.5 g). ¹HNMR (400 MHz, DMSO-d₆): δ 13.08 (bs, 1 H), 8.75 (s, 1 H), 8.73 (s, 1 H), 7.26 (s, 1 H), 3.84 (s, 3 H). LC-MS (ES) m/z= 202. 1[M+H]⁺.

### Step 7: 4-Chloro-6-Methoxy-1,7-Naphthyridine-3-Carbonitrile

Procedure: To a suspension of 4-hydroxy-6-methoxy-1,7-naphthyridine-3-carbonitrile (0.450 g, 2.236 mmol) in acetonitrile (20 mL) was added phosphorus oxychloride (0.62 mL, 6.704 mmol) and diisopropylethylamine (2.34 mL, 13.415 mmol). Solution heated to reflux for 3 h. The progress of the reaction was monitored by TLC. Reaction mixture was concentrated to give crude. The Crude was purified by gradient column chromatography using ethyl acetate in n-hexane to afford 4-chloro-6-methoxy-1,7-naphthyridine-3-carbonitrile as yellow solid (0.250 g, 50.91 %). ¹HNMR (400 MHz, CDC13): δ 9.31 (s, 1 H), 8.82 (s, 1 H), 7.36 (s, 1 H), 4.12 (s, 3 H). LC-MS (ES) m/z = 220.1 [M+H]⁺.

### Step 8: Tert-Butyl (4-(3-Cyano-6-Methoxy-1,7-Naphthyridin-4-yl)Benzyl)Carbamate

Procedure: To a stirred solution of 4-chloro-6-methoxy-1,7-naphthyridine-3-carbonitrile (0.2 g, 0.910 mmol) in toluene / water (16 mL / 2 mL) was added sodium bicarbonate (0.229 g, 2.731 mmol), (4-(((tert-butoxycarbonyl)amino)methyl)phenyl)boronic acid (0.342 g, 1.365 mmol) and mixture was degassed with argon with 10 min. Tetrakis(triphenylphosphine)palladium(0) (0.105 g, 0.09 mmol) was added to above solution and heated at 110° C. for 12 h. The progress of the reaction was monitored by TLC. Reaction mixture was cooled and diluted with water, extracted using ethyl acetate (3 x 20 mL) dried over sodium sulphate, concentrated. Crude product was purified by gradient column chromatography using ethyl acetate in n-hexane to afford tert-butyl (4-(3-cyano-6-methoxy-1,7-naphthyridin-4-yl)benzyl)carbamate as yellow solid (0.325 g, 91.54 %). LC-MS (ES) m/z = 391.4 [M+H]⁺.

### Step 9: 4-(4-(Aminomethyl)Phenyl)-6-Methoxy-1,7-Naphthyridine-3-Carbonitrile Hydrochloride

Procedure: To a stirred solution tert-butyl (4-(3-cyano-6-methoxy-1,7-naphthyridin-4-yl)benzyl) carbamate (0.250 g, 0.640 mmol) in dichloromethane (25 mL ) was added HCl. dioxane (1.6 mL, 4M solution) at 0° C. and the mixture was stirred at room temperature for 15 h. The progress of the reaction was monitored by TLC. Reaction mixture was filtered and solid washed with dichloromethane, dried under vacuum to afford 4-(4-(aminomethyl)phenyl)-6-methoxy-1,7-naphthyridine-3-carbonitrile hydrochloride as yellow solid (0.150 g, 71.77 %). LC-MS (ES) m/z = 291 [M+H]⁺.

### Step 10: N-(4-(3-Cyano-6-Methoxy-1,7-Naphthyridin-4-yl)Benzyl)Cyclopropanesulfonamide

Procedure: To a stirred solution of 4-(4-(aminomethyl)phenyl)-6-methoxy-1,7-naphthyridine-3-carbonitrile hydrochloride (0.075 g, 0.229 mmol) and N,N-diisopropylethylamine (0.2 mL, 1.145 mmol) in acetonitrile (6 mL) was added cyclopropanesulfonyl chloride (0.035 mL, 0.341 mmol) at 0° C. and the mixture was stirred at room temperature for 8 h. The progress of the reaction was monitored by TLC. Solvent was concentrated, and residue was diluted with water, extracted with ethyl acetate (3 x 10 mL), dried over sodium sulphate, concentrated. The crude was purified by gradient column chromatography using methanol in dichloromethane to afford N-(4-(3-cyano-6-methoxy-1,7-naphthyridin-4-yl)benzyl)cyclopropanesulfonamide as off-white solid (0.028 g, 31.11 %). ¹HNMR (400 MHz, DMSO-d₆): δ 9.33 (s, 1 H), 9.13 (s, 1 H), 7.77 (t, J = 6.8 Hz, 1 H), 7.64 (d, J = 8 Hz, 2 H), 7.59 (d, J = 8 Hz, 2 H), 6.75 (s, 1 H), 4.34 (d, J = 6 Hz, 2 H), 3.93 (s, 3 H), 2.48 (m, 1 H), 0.89 - 0.87 (m, 4 H), LC-MS (ES) m/z = 393.0 [M+H]⁺ HPLC purity 99.88 %.

Example 84: N-(4-((6-fluoro-7-methoxyquinolin-4-yl)amino) phenyl) cyclopropane sulfonamide

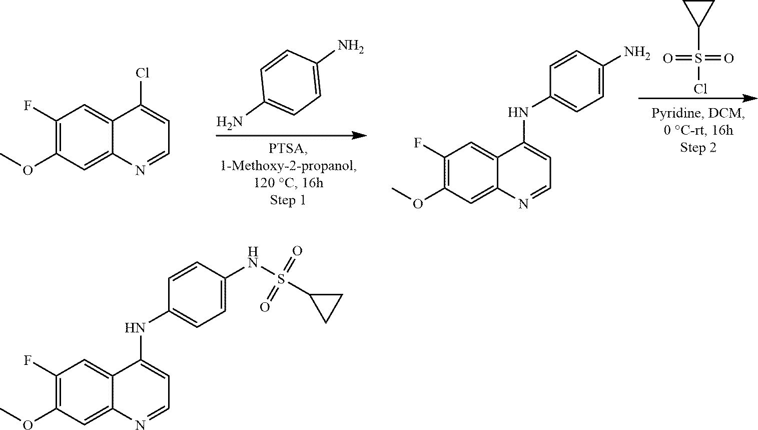

### Step 1 N1-(6-Fluoro-7-Methoxyquinolin-4-yl)Benzene-1,4-Diamine

Procedure: To a stirred solution of 4-chloro-6-fluoro-7-methoxyquinoline (0.08 g, 0.37 mmol) in 1-methoxy-2-propanol (5 mL) was added benzene-1,4-diamine (0.045 g, 0.41 mmol), and p-toluene sulfonic acid (0.036 g, 0.18 mmol). The resulting mixture was stirred for 16 h at 120 C. Progress of the reaction was monitored by TLC. Reaction mixture was evaporated resulting residue was dissolved in 15% methanol in dichloromethane (30 mL), washed with water (5 mL) followed by brine (5 mL), dried over anhydrous sodium sulphate, filtered and evaporated under reduced pressure to afford crude compound N1-(6-fluoro-7-methoxyquinolin-4-yl)benzene-1,4-diamine as yellow solid (0.12 g, 75%). The crude compound was directly used for next step without further purification. LCMS (ES) m/z = 284.0 [M+H]⁺.

### Step 2: N-(4-((6-Fluoro-7-Methoxyquinolin-4-yl)Amino) Phenyl) Cyclopropane Sulfonamide

Procedure: To a stirred solution of N1-(6-fluoro-7-methoxyquinolin-4-yl)benzene-1,4-diamine (0.08 g, 0.28 mmol) in dichloromethane (5 mL) was added cyclopropanesulfonyl chloride (0.043 mL, 0.42 mmol), and pyridine (0.068 mL, 0.85 mmol) at 0 C. Reaction mixture was stirred at room temperature for 16 h, reaction was monitored by TLC. The reaction mixture was evaporated under reduced pressure. The crude residue was purified by combiflash using 3-5% methanol in dichloromethane to afford N-(4-((6-fluoro-7-methoxyquinolin-4-yl)amino)phenyl)cyclopropanesulfonamide as off-white solid (0.02 g, 13% over 2 steps). ¹HNMR (400 MHz, DMSO-d₆): δ 9.61 (s, 1 H), 8.70 (s, 1 H), 8.34 (d, J = 5.2 Hz, 1 H), 8.17 (d, J = 13.2 Hz, 1 H), 7.41 (d, J = 8.0 Hz, 1 H), 7.32 - 7.22 (m, 4 H), 6.73 (d, J = 4.8 Hz, 1 H), 3.97 (s, 3 H), 2.61 - 2.55 (m, 1 H), 0.93 -0.84 (m, 4 H); LCMS (ES) m/z = 388.3 [M+H]⁺; HPLC purity: 98.94%.

Example 85: N-(4-(6,7-dimethoxyquinoxalin-2-yl) phenyl) cyclopropane sulfonamide:

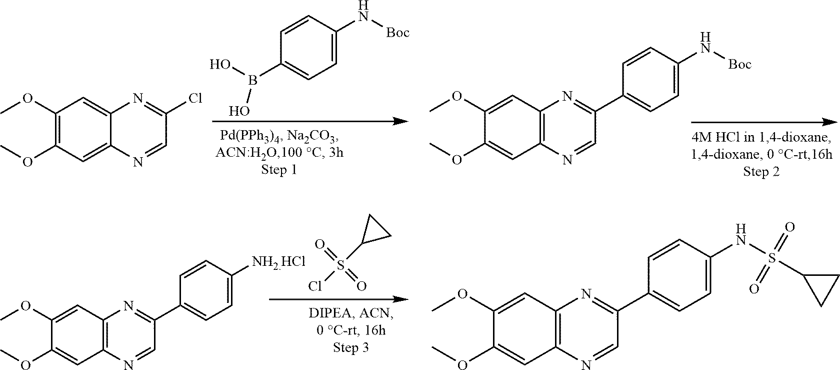

### Step 1 Tert-Butyl (4-(6,7-Dimethoxyquinoxalin-2-yl)Phenyl)Carbamate

Procedure: To a stirred solution of 2-chloro-6,7-dimethoxyquinoxaline (0.2 g, 0.89 mmol) in acetonitrile (9 mL) and water (3 mL) was added (4-((tert-butoxycarbonyl)amino)phenyl)boronic acid (0.23 g, 0.98 mmol), and sodium carbonate (0.28 g, 2.67 mmol). The resulting mixture was degassed for 15 min with argon and added Pd(PPh₃)₄ (0.051 g, 0.044 mmol) degassed for another 10 min. Resulting mixture was stirred for 3 h at 100 C. Progress of the reaction was monitored by TLC. Reaction mixture was filtered through celite bed and filtrate was diluted with ethyl acetate (20mL), washed with water (2 x 10 mL) followed by brine (10 mL), dried over anhydrous sodium sulphate, filtered and evaporated under reduced pressure to give crude product. The crude product was purified by combiflash using 40% ethyl acetate in hexane to afford tert-butyl (4-(6,7-dimethoxyquinoxalin-2-yl)phenyl)carbamate as off-white solid (0.4 g, 73%).¹HNMR (400 MHz, DMSO-d₆): δ 9.58 (s, 1 H), 9.26 (s, 1 H), 8.17 (d, J= 8.8 Hz, 2 H), 7.62 (d, J= 8.8 Hz, 2 H), 7.40 (d, J= 5.2 Hz, 2 H), 4.02 (s, 3 H), 4.00 (s, 3 H), 1.49 (s, 9 H); LCMS (ES) m/z = 382.1 [M+H]⁺ .

### Step 2 Synthesis of 4-(6,7-Dimethoxyquinoxalin-2-yl)Aniline Hydrochloride

Procedure: To a stirred solution of tert-butyl (4-(6,7-dimethoxyquinoxalin-2-yl)phenyl) carbamate (0.3 g, 0.78 mmol) in 1,4-dioxane (5 mL) was added 4M HCl in 1,4-dioxane (10 mL). Reaction mixture was stirred at room temperature for 6 h and monitored by TLC. The reaction mixture was evaporated under reduced pressure and co-distilled with toluene (twice) and dried to give 4-(6,7-dimethoxyquinoxalin-2-yl)aniline hydrochloride as brown solid (0.24 g, Quantitative). LC-MS (ES) m/z = 282.1 [M+H] ⁺.

### Step 3: Synthesis of N-(4-(6,7-Dimethoxyquinoxalin-2-yl)Phenyl)Cyclopropanesulfonamide

Procedure: To a stirred solution of 4-(6,7-dimethoxyquinoxalin-2-yl)aniline hydrochloride (0.12 g, 0.37 mmol) in acetonitrile (10 mL) was added cyclopropane sulfonyl chloride (0.057 mL, 0.56 mmol), and N,N-Diisopropylethylamine (0.091 mL, 1.13 mmol) at 0° C. Reaction mixture was stirred at room temperature for 16 h. The reaction was monitored by TLC. The reaction mixture was evaporated under reduced pressure to give crude product. The crude product was purified by combiflash using 60-65% ethyl acetate in hexane to afford N-(4-(6,7-dimethoxyquinoxalin-2-yl)phenyl)cyclopropanesulfonamide as off-white solid (0.028 g, 20%).¹HNMR (400 MHz, DMSO-d₆): δ 10.01 (s, 1 H), 9.28 (s, 1 H), 8.22 (d, J= 8.4 Hz, 2 H), 7.41 - 7.37 (m, 4 H), 3.98 (s, 3 H), 3.97 (s, 3 H), 2.70 -2.65 (m, 1 H), 0.97 - 0.95 (m, 4 H); LCMS (ES) m/z = 383.9 [M-H]⁺; HPLC purity: 99.01%.

Examples 86-324, 329, 330, and 345-421: Examples 86-324, 329, 330, and 345-421 were synthesized as described for examples 1-63 and 81-85.

| Ex. | LCMS [M+H]⁺ | ¹H-NMR (400 MHz, DMSO-d₆) δ |
|---|---|---|
| 86 | 437.1 | 10.32 (s, 1 H), 8.43 (d, J= 4.8 Hz, 1 H), 7.75 (d, J= 6.8 Hz, 2 H), 7.65-7.54 (m, 3 H), 7.43 (s, 1 H), 7.36 (s, 1 H), 7.20 - 7.10 (m, 4 H), 6.33 (d, J = 5.2 Hz, 1 H), 3.92 (s, 3 H), 3.88 (s, 3 H). |
| 87 | 400.3 | 9.90 - 9.60 (m, 2 H), 8.29 (d, J= 6.4 Hz, 1 H), 7.85 (s, 1 H), 7.35 (s, 4 H), 7.28 (s, 1 H), 6.67 (d, J= 6 Hz, 1 H), 3.94 (s, 6 H), 2.50 (m, 1 H), 0.93 (m, 4 H). |
| 88 | 425.3 | 9.71 (s, 1 H), 9.7-9.6 (bs, 1 H), 8.48 (s, 1 H), 7.79 (s, 1 H), 7.31 - 7.24 (m, 5 H), 3.94 (s, 3 H), 3.91 (s, 3 H), 2.57- 2.56 (m, 1 H), 0.90 - 088 (m, 4 H). |
| 89 | 437.0 | 9.91 (s, 1 H), 8.49 (d, J= 5.2 Hz, 1 H), 7.61 (d, J= 7.2 Hz, 1 H), 7.53 - 7.48 (m, 2 H), 7.40 (s, 1 H), 6.58 (d, J= 5.2 Hz, 1 H), 3.93 (s, 3 H), 3.92 (s, 3H), 2.77 (m, 1 H) 0.99 - 0.93 (m, , 4 H). |
| 90 | 407 | 9.81 (s, 1 H), 8.70 (d, J = 5.2 Hz, 1 H), 8.36 (d, J= 9.2 Hz, 1 H), 7.71 (s, 1 H), |
| | | 7.54 - 7.45 (m, 2 H), 7.40 - 7.32 (m, 2 H), 7.30 - 7.24 (m, 2 H), 6.57 (d, J = 5.2 Hz, 1 H), 2.70 - 2.60 (m, 1 H) 1.00 - 0.90 (m, 4 H). |
| 91 | 418 | 9.48 (s, 1 H), 9.18 (br, 1 H), 8.35 (d, J= 5.6 Hz, 1 H), 7.67 (s, 1 H), 7.41 (t, J = 8.8 Hz, 1 H), 7.28 (s, 1 H), 7.24 (d, J= 11.6 Hz, 1 H), 7.16 (d, J= 8.8 Hz, 1 H), 6.92 (d, J= 5.6 Hz, 1 H), 3.93 (s, 3 H), 3.91 (s, 3 H), 2.63 (m, 1 H), 1.00 -0.90 (m, 2 H), 0.90 - 0.82 (m, 2 H) . |
| 92 | 412.9 | 9.70 (s, 1 H), 8.12 (d, J = 8 Hz, 1 H), 7.51 (s, 1 H), 7.25 - 7.15 (m, 4 H), 6.99 (s, 1 H), 6.11 (d, J = 7.6 Hz, 1 H), 5.48 (s, 2 H), 3.80 (s, 3 H), 3.75 (s, 3 H), 2.58 - 2.48 (m, 1 H), 0.90 - 0.80 (m, 4 H). |
| 93 | 402.3 | 9.75 (s, 1 H), 8.53 (s, 1 H), 7.53 (s, 1 H), 7.36 (s, 1 H), 7.31 (d, J= 9.2 Hz, 2 H), 7.26 (d, J= 8.8 Hz, 2 H), 3.97 (s, 3 H), 3.95 (s, 3 H), 2.65 - 2.60 (m, 1 H), 0.90 - 0.80 (m, 4 H). |
| 94 | 426.3 | 9.68 (bs, 1 H), 8.85 (s, 1 H), 7.51 (s, 1 H), 7.32 (s, 1 H), 7.26 (d, J= 8.8 Hz, 2 H), 7.14 (d, J = 8.8 Hz, 2 H), 3.98 (s, 3 H), 3.83 (s,3 H), 2.57 - 2.54 (m, 1 H), 0.89 - 088 (m, 4 H). |
| 95 | 418.3 | 9.36 (s,1 H), 8.58 (s,1 H), 8.49(d, J= 3.6 Hz, 1 H),7.48 (s,1 H), 7.31(s,1 H), 7.13 (d, J= 8.8 Hz, 2 H), 6.89 (d, J= 7.2 Hz, 2 H), 3.89 (s, 3 H), 3.85 (s, 3 H), 2.058 (s, 1 H), 0.9-0.86 (m, 4 H). |
| 96 | 426.0 | 9.25 (bs, 1 H), 8.56 (d, J = 5.2 Hz, 1 H), 7.36 (s, 1 H), 7.18 - 7.15 (m, 2 H), 7.08 (s, 1 H), 6.90 - 6.87 (m, 1 H), 6.35 (d, J = 8.4, 1 H), 4.05 (t, J = 8 Hz, 2 H), 3.92 (s, 3 H), 3.73 ( s, 3 H), 3.17 (t, J= 8.4 Hz, 2 H), 2.53 - 2.48 (m, 1 H), 0.89 - 085 (m, 4 H). |
| 97 | 450.3 | 9.00 (s, 1 H), 7.55 (s, 1 H), 7.44 - 7.40 (s, 3 H), 6.94 (s, 1 H), 4.53 (q, J= 16.4 Hz, 2 H), 3.99 (s, 3 H), 3.73 (s, 3 H), 3.64 - 3.57 (m, 2 H), 3.11 - 3.05 (m, 2 H), 2.66 - 2.61 (m, 1 H), 0.98 -0.94 (m, 4 H); |
| 98 | 457.4 | 8.56 (s, 1 H), 7.35 (s, 1 H), 7.23 (s, 1 H), 3.93 (s, 6 H), 3.62 - 3.51 (m, 4H), 3.42 (t, J= 6.8 Hz, 2 H), 2.74 - 2.71 (m, 1 H), 1.94 - 1.83 (m, 8 H), 0.97 -0.95 (m, 4 H) |
| 99 | 370.1 | 9.6 (br s, 1 H), 8.81 (s, 1 H), 8.33 (s, 1 H), 8.24 (d, J= 9.2 Hz, 1 H), 7.26 - 7.22 (m, 5 H), 7.13 (d, J = 9.2 Hz, 1 H), 6.68 (s, 1 H), 3.87 (s, 3 H), 2.58 (s, 1 H), 0.911 (s, 4 H). |
| 100 | 419.3 | 10.22 (s, 1 H), 8.75 (d, J= 5.6 Hz, 1 H), 7.69 (s, 1 H), 7.64-7.44 (m, 2 H), 7.33 (d, J= 11.6 Hz, 1 H), 7.22 (d, J= 8.4 Hz, 1 H), 6.88 (br.s., 1 H), 4.02 (s, 3H), 4.01 (s, 3 H), 2.85-2.75 (m, 1 H), 1.00-0.99 (m, 4 H) |
| 101 | 419 | 9.63 (s, 1 H), 8.50 (d, J= 4.8 Hz, 1 H), 7.51-7.32 (m, 4 H), 7.09 (d, J= 8.8 Hz, 1 H), 6.60 (d, J= 5.2 Hz, 1 H), 3.93 (s, 3 H), 3.90 (s, 3 H), 2.71-2.60 (m, 1H), 0.96-0.87 (m, 2 H), 0.76-0.68 (m, 2 H) |
| 102 | 372.1 | δ 9.70 (bs, 1 H), 8.59 (d, J = 5.2 Hz, 1 H), 8.25 (d, J= 8.8 Hz, 1 H), 7.32-7.26 (m, 3 H), 7.17 (d, J = 8.8 Hz, 2 H), 6.91 (d, J = 8.8 Hz, 1 H), 3.89 (s, 3 H), 2.61 - 2.57 (m, 1 H), 0.93-0.92 (m, 4 H). |
| 103 | 371.1 | δ 9.62 (bs, 1 H), 8.62 (bs, 1 H), 8.33 (d, J = 5.2 Hz, 1 H), 8.10 (d, J = 9.2 Hz, 1 H), 7.39 (d, J = 8.8 Hz, 2 H), 7.28 (d, J = 8 Hz, 2 H), 7.19 (d, J = 9.2 Hz, 1 H), 6.98 (d, J= 5.2 Hz, 1 H), 4.08 (s, 3 H), 2.61 - 2.55 (m, 1 H), 0.93-0.91 (m, 4 H). |
| 104 | 370.9 | 9.77 (s, 1 H), 8.59 (d, J = 4.4 Hz, 1 H), 8.17 (d, J= 9.6 Hz, 1 H), 7.39 - 7.34 (m, 3 H), 7.28 - 7.23 (m, 3 H), 6.44 (d, J= 5.2 Hz, 1 H), 3.92 (s, 3 H), 2.65 -2.60 (m, 1 H), 0.95 - 0.93 (m, 4 H) |
| 105 | 343.2 | 9.22 (d, J= 2.4 Hz, 1 H), 8.84 (s, 1 H), 8.80 - 8.78 (m, 1 H), 7.80 - 7.77 (m, 1 H), 7.31(br s, 4 H), 2.65 - 2.59 (m, 1 H), 0.96 - 0.92 (m, 4 H) |
| 106 | 414.0 | 9.59 (s, 1 H), 8.11 (d, J= 5.6 Hz, 1 H), 7.57 (s, 2 H), 7.32 (d, J= 8.4 Hz, 2 H), 7.14-7.17 (m, 3 H), 6.21 (d, J = 5.6 Hz, 1 H), 4.46 (d, J = 5.6 Hz, 2 H), 3.87 (d, J= 16.8 Hz, 6 H), 1.89-2.05 (m, 1 H), 1.22 (s, 2H), 0.82-0.89 (m, 2 H). |
| 107 | 415.3 | 9.86 (s, 1 H), 8.46 (d, J = 5.6 Hz, 1 H), 7.48(s, 1 H), 7.36 (d, J= 11.2 Hz, 3 H), 7.23(d, J = 8.4 Hz, 2 H), 6.42(d, J= 5.2 Hz, 1 H), 3.92 (d, J= 7.2 Hz, 6 |
| Ex. | LCMS [M+H]⁺ | ¹H-NMR (400 MHz, DMSO-d₆) δ |
| | | H), 1.41 (s, 3H), 1.07 (s, 2 H), 0.75 (s, 2 H). |
| 108 | 436.0 | 8.48 (bs, 1 H), 7.17 (s, 1 H), 7.12 (s, 1 H), 7.08 (s, 1 H), 6.85 (bs, 1 H), 4.11 (d, J= 12.4 Hz, 2 H), 3.89 - 3.87 (m, 6 H), 3.02 - 2.96 (m, 2 H) 2.88 - 2.87 (m, 2 H), 2.26 (bs, 1 H), 1.79 - 1.76 (m , 2 H), 1.67 (bs, 1 H), 1.47 - 1.46 (m, 2 H), 1.38 - 1.32 (m, 2 H), 0.50 - 0.46 (m 4 H). |
| 109 | 433.4 | 8.50 (bs, 1 H), 7.19 (s, 1 H), 7.11 (s, 1 H), 3.91 (s, 3 H), 3.89 (s, 3 H), 3.66 -3.55 (m, 4 H) 3.41 - 3.37 (m, 2 H), 3.23 (s, 2 H), 2.72 - 2.68 (m ,1 H), 1.89 -1.85 (m, 2 H), 1.78 - 1.73 (m, 4 H), 0.95 - 0.94 (m 4 H). |
| 110 | 437.0 | 9.49 (bs, 1 H), 8.55 (d, J = 5.6 Hz, 1 H), 7.40 (d, J = 8.8 Hz, 2 H), 7.20 (d, J = 8 Hz, 2 H), 6.77 (d, J= 4.8 Hz, 1 H), 3.94 - 3.89 (m, 6 H), 2.68 - 2.65 (m, 1 H), 0.99 - 0.97 (m 2 H), 0.94 - 0.89 (m, 2 H). |
| 111 | 440.1 | 9.12 (bs, 1 H), 8.28 (d, J = 5.2 Hz, 1 H), 7.73 (s, 1 H), 7.57 (d, J= 8.8 Hz, 1 H), 7.25 (s, 1 H), 7.16 (s, 2 H), 6.76 (d, J= 5.2 Hz, 1 H), 3.92 - 3.91 (m, 6 H), 3.72 (bs, 2 H), 2.84 (bs, 2 H), 2.74 (s, 1 H), 1.99 (bs, 2 H), 0.95 - 0.91 (m, 4 H). |
| 112 | 389.3 | 9.79 (s, 1 H), 8.59 (d, J = 5.2 Hz, 1 H), 7.95 (d, J = 12 Hz, 1 H), 7.59 (d, J= 8.4 Hz, 1 H), 7.35 (d, J = 8.8 Hz, 2 H), 7.24 (d, J = 8.8 Hz, 2 H), 6.50 (d, J = 4.8 Hz, 1 H), 4.01 (s, 3 H), 2.65 - 2.60 (m, 1 H), 0.95 - 0.93 (m, 4 H) |
| 113 | 361.1 | |
| 114 | 376.1 | |
| 115 | 345.2 | |
| 116 | 457.3 | |
| 117 | 360.3 | |
| 118 | 388.2 | |
| 119 | 332.2 | |
| 120 | 363.1 | 8.29 (s, 1 H), 7.63-7.61 (m, 2 H), 7.58-7.56 (m, 2 H), 7.29 (s, 1 H), 7.15 (b, 1 H), 7.04 (s, 1 H), 6.58 (b, 2 H), 4.16 (s, 2 H), 3.80-3.78 (m, 6 H). |
| 121 | 401.0 | 9.09 (s, 1 H), 7.64 - 7.26 (m, 2 H), 7.42 (s, 1 H), 7.38 (d, J= 8 Hz, 1 H), 7.32 (s, 1 H), 6.94 (bs, 2 H), 4.31 (s, 2 H), 3.99 (s, 3 H), 3.82 (s, 3 H), 3.33 (t, J= 6 Hz, 2 H), 3.03 (t, J = 5.2 Hz, 2 H). |
| 122 | 401.1 | 9.08 (s, 1 H), 7.63 (s, 1 H), 7.59 (d, J = 7.6 Hz, 1 H), 7.41 - 7.39 (m, 2 H), 7.33 (s, 1 H), 6.93 (d, J= 7.2 Hz, 1 H), 6.60 (bs, 2 H), 4.15 - 4.13 (m, 1 H), 3.99 (s, 3 H), 3.82 (s, 3 H), 3.33 - 3.31 (m, 2 H), 3.01 - 2.95 (m, 2 H). |
| 123 | 401.2 | 6.63 (s, 1 H), 6.42-6.37 (m, 3 H), 6.22 (s, 1 H), 5.14-5.13 (m, 1 H), 4.05-3.95 (m, 2 H), 3.77 (s, 3 H), 3.62 (s, 3 H), 2.90-2.78(m, 4 H), 2.69 (d, J = 7.2 Hz, 3 H), 1.65-1.62 (m, 3 H), 1.40-1.39 (m, 2 H), 1.09-1.04 (m, 2 H) |
| 124 | 373.3 | |
| 125 | 376.1 | |
| 126 | 390.1 | 9.39 (s, 1 H), 7.81 (d, J= 9.6 Hz, 1 H), 7.24 (s, 1 H), 7.19 (d, J = 7.2 Hz, 2 H), 7.07 (d, J= 8.4 Hz, 2 H), 6.98 (s, 2 H), 6.90 (s, 1 H), 6.52 (d, J = 9.6 Hz, 1 H), 5.46 (s, 2 H), 3.76 (s, 3 H), 3.74 (s, 3 H) |
| 127 | 323.1 | |
| 128 | 319.1 | 8.97 (s, 1 H), 8.65 (m, 1 H), 7.68 (d, J = 8.0 Hz, 2 H), 7.54 (d, J = 8.0 Hz, 2 H), 7.15(t, J= 6.4 Hz, 1 H), 6.65(s, 2 H), 4.19(d, J= 6.4 Hz, 2 H), 3.60(s, 3 H). |
| 129 | 397.2 | 7.43 (s, 1 H), 7.06 (s, 1 H), 6.45-6.41 (m, 3 H), 4.45 (d, J= 12.4 Hz, 1 H), 3.83 (s, 3 H), 3.82 (s, 3 H), 3.01-2.86 (m, 3 H), 2.75-2.48 (m, 2 H), 1.73-1.56 (s, 3 H), 1.40-1.39 (m, 2 H), 1.10-1.08 (m, 2 H) |
| 130 | 343.2 | 8.70 (bs, 1 H), 8.50 (s, 1 H), 7.23 (s, 1 H), 6.42 - 6.45 (m, 3 H), 4.40 (bs, 2 H), 2.88-2.90 (m, 4 H), 2.76 (d, J= 4.4 Hz, 3 H), 1.71-1.75 (m, 3 H), 1.38 - 1.40 (m, 2 H), 1.05 - 1.19 (m, 2 H). |
| 131 | 415.1 | 9.10 (s, 1 H), 7.58-7.56 (m, 2 H), 7.43(s, 1 H), 7.36(s, 1 H), 7.29(d, J= 7.6 Hz, 1 H), 6.72 (d, J = 6.4 Hz, 1 H), 6.58 (s, 2 H), 4.01(s, 3 H), 3.84(s, 3 H), 3.65-3.55(m, 1 H), 3.21-3.16(m, 1 H), 3.04-2.90(m, 2 H), 2.84-2.78(m, 1 H), 2.20-2.10(m, 1 H), 1.80-1.70(m, 1 H). |
| 132 | 466.2 | |
| 133 | 372.2 | 9.61 (s, 1 H), 7.33 (s, 1 H), 7.06 (d, J= 6.8 Hz, 1 H), 6.84 (d, J= 8.4 Hz, 1H), 6.43-6.40 (m, 3 H), 3.70 (s, 3 H), 3.69 (s, 3 H), 2.72-2.66 (m, 2 H), 2.31-2.21 (m, 1 H), 1.91-1.72 (m, 4 H), 1.40-1.37 (m, 3 H), 0.91-0.88 (m, 2 H) |
| 134 | 375 | |
| 135 | 339.2 | 8.14 (s, 1 H), 8.08 (d, J = 5.6 Hz, 1 H), 6.46 - 6.43 (m, 3 H), 6.39 (d, J= 5.2 Hz, 1 H), 4.12 (d, J = 13.2 Hz, 2 H), 3.91 (s, 3 H), 3.07 (t, J = 12.4 Hz, 2H), 2.90 (d, J = 6.4 Hz, 2 H); 1.77 (d, J = 12.8 Hz, 2 H), 1.74 - 1.64 (m, 1 H), 1.41 (d, J = 6.4 Hz, 2 H); 1.23 - 1.15 (m, 2 H); |
| 136 | 370.2 | 9.05 (s, 1 H), 8.05 (d, J= 8.0 Hz, 2 H), 7.81 (d, J= 8.0 Hz, 2 H), 7.57 (s, 3 H), 6.83 (s, 1 H), 4.00 (s, 3 H), 3.72 (s, 3 H) |
| 137 | 416.1 | 9.33 (s, 1 H), 8.51 (s, 1 H), 7.22 - 7.20 (m, 2 H), 7.14 (d,J= 8.4 Hz, 1 H), 7.03 - 6.96 (m, 4 H), 4.73 (s, 2 H), 3.92 (s, 6 H), 3.87 (m, 2 H), 3.04 (s, 2 H) |
| 138 | 417.0 | 8.38 (s, 1 H), 7.82 (s, 1 H), 6.43 - 6.41 (m, 3H), 3.90 (s, 3 H), 3.72 - 3.68 (m, 2 H), 2.94 - 2.83 (m, 4 H), 1.78 - 1.75 (m, 2 H), 1.59 (m, 1 H), 1.48 - 1.45 (m, 2 H), 1.36 - 1.27 (m, 2H). |
| 139 | 424.0 | |
| 140 | 404 | |
| 141 | 355.7 | |
| 142 | 438.1 | 8.48 (s, 1 H), 7.17 (s, 1 H), 7.09 (s, 1 H), 6.66- 6.65 (m, 2 H), 4.12 (d, J= 12.8 Hz, 2 H), 3.90 (s, 3 H), 3.88 (s, 3 H), 3.26 - 3.23 (m, 1 H), 2.99 (t, J= 12 Hz, 2 H), 2.86 (d, J = 6.4 Hz, 2 H); 1.78 (d, J = 12 Hz, 2 H), 1.72 - 1.65 (m, 1 H), 1.47 -1.33 (m, 4 H) 1.08 (s, 3 H), 1.07(s, 3 H); |
| 143 | 424 | 8.45(s, 1 H), 7.17(s, 1H), 7.10-7.08(m, 2 H), 4.11(d, J = 12.8 Hz, 2 H), 3.90(s, 3 H), 3.88(s, 3 H), 3.02-2.94(m, 4 H), 2.64(s, 6 H), 1.79-1.76(m, 2 H), 1.20-1.10(m, 1 H), 1.45-1.43(m, 2 H), 1.38-1.29(m, 2 H). |
| 144 | 375.1 | 9.09 (s, 1 H), 7.78(d, J= 7.6 Hz, 2 H), 7.56(d, J= 8.0 Hz, 2 H), 7.41(s, 1 H), 7.31(s, 1 H), 7.15 (bs, 1 H), 6.65(s, 2 H), 4.19(d, J= 6.0 Hz, 2 H) 3.99(s, 3 H), 3.81(s, 3 H). |
| 145 | 389.1 | 9.09 (s, 1 H), 7.76(d, J= 8.0 Hz, 2H), 7.45(d, J= 8.0 Hz, 2 H), 7.41 (s, 1H), 7.33(s, 1 H), 6.61(bs, 1 H), 6.54(s, 2 H), 3.98(s, 3 H), 3.82(s, 3H), 3.19-3.14(m, 2 H), 2.90(t, J = 7.2 Hz, 2 H). |
| 146 | 449.1 | |
| 147 | 465.1 | |
| 148 | 400.1 | |
| 149 | 425.5 | |
| 150 | 341.1 | |
| 151 | 390 | 9.48 (s, 1 H), 8.05 (d, J= 7.6 Hz, 1 H), 7.50 (s, 1 H), 7.19 (d, J = 8.4 Hz, 2H), 7.09 (d, J = 8.4 Hz, 2 H), 7.04 - 6.95 (m, 3 H), 6.01 (d, J = 7.6 Hz, 1 H), 5.40 (s, 2 H), 3.79 (s, 3H), 3.76 (s, 3 H) |
| 152 | 409.0 | 7.29 (d, J = 8.4 Hz, 1 H), 7.16 (d, J = 8.8 Hz, 1 H), 7.12 (s, 1 H), 6.43(s, 2 H), 6.11 (t, J = 5.2 Hz, 1 H), 3.82 (d, J = 8.4 Hz, 6 H), 2.85-2.91(m, 4 H), 2.30-2.47(m, 5 H), 1.21(s, 1 H). |
| 153 | 436.1 | 10.43 (bs, 1 H), 9.96 (s, 1 H), 8.28 (d, J = 5.2 Hz, 1 H), 7.87 (s, 1 H), 7.77 (d, J= 7.2 Hz, 2 H), 7.61 (d, J =6.4 Hz, 2 H), 7.56 (d, J =6.0 Hz, 1 H), 7.28 (d, J =8 Hz, 3 H), 7.22 (d, J =8 Hz, 2 H), 6.56 (d, J =5.6 Hz, 1 H), 3.93 (d, J =4 Hz, 6 H). |
| 154 | 423.0 | |
| 155 | 458.1 | |
| 156 | 404.1 | 9.31(s, 1 H), 8.34(s, 1 H), 7.98(bs, 1 H), 7.55(s, 1 H), 7.16-7.14(m, 2 H), 7.09-7.06(m, 3 H), 6.97(s, 2 H), 3.87(s, 3 H), 3.86(s, 3 H), 3.70-3.6(m, 2 H), 2.90-2.80(m, 2 H). |
| 157 | 404.2 | 9.10 (s, 1 H), 8.51 (s, 1 H), 7.69 (d, J = 7.6 Hz, 1 H), 7.50 - 7.47 (m, 2 H), 7.20 |
| | | (s, 2 H), 6.36 (m, 1 H), 4.97 (s, 2 H), 4.35 (m, 2 H), 3.93 (s, 3 H), 3.92 (s, 3 H), 3.88 (m, 2 H), 2.83 (m, 2 H). |
| 158 | 348.2 | |
| 159 | 361.2 | |
| 160 | 407.0 | 8.47 (d, J = 4.8 Hz, 1 H), 7.29 (s, 1 H), 7.17 (m, 1 H), 6.86 (d, J= 4.8 Hz, 1H), 6.72 (m, 2 H), 3.89 (s, 6 H), 3.22 (t, J = 6.4 Hz, 2 H), 3.18 - 3.02 (m, 6 H), 1.82 - 1.79 (m, 6H); |
| 161 | 376.1 | 10.03(bs, 1 H), 8.77(s, 1 H), 7.88(s, 1 H), 7.62(s, 1 H), 7.50(d, J= 6.8 Hz, 2H), 7.34-7.27(m, 2 H), 7.15(s, 1 H), 4.92(d, J =5.2 Hz, 2 H), 3.95(s, 3 H), 3.92(m, 3 H). |
| 162 | 429 | |
| 163 | 393.2 | |
| 164 | 381.0 | 8.50 (s, 1 H), 7.18 (s, 1 H), 7.10 (s, 1 H), 6.73 (s, 2 H), 4.13 (d, J = 12.8 Hz, 2 H), 3.91 (s, 3 H), 3.89 (s, 3 H), 3.04 - 2.98 (m, 4 H), 1.81 (d, J = 12.4 Hz, 2 H), 1.72 - 1.68 (m, 3 H), 1.42 - 1.32 (m, 2 H); |
| 165 | 391 | 9.45 (s, 1 H), 8.42 (s, 1 H), 7.45 (s, 1 H), 7.27 (d, J= 8.0 Hz, 2 H), 7.14 - 7.08 (m, 3 H), 7.02 (s, 2 H), 5.08 (s, 2 H), 3.88 (s, 3 H), 3.85 (m, 3 H) |
| 166 | 363.0 | |
| 167 | 372.2 | 8.72 (s, 1 H), 8.06 (d, J = 8.4 Hz, 1 H), 7.95 (d, J = 8.0 Hz, 1 H), 7.82 (t, J = 7.2 Hz, 1 H),7.62 (t, J= 7.2 Hz, 1 H), 6.76 (s, 2 H), 3.67 - 3.62 (m, 4 H), 3.24 (t, J= 7.2 Hz, 2 H), 3.14 (s, 2 H), 1.87 - 1.81 (m, 6 H); |
| 168 | 410.0 | 8.5 (s, 1 H), 7.18 (s, 1 H), 7.09(s, 1 H), 6.78 - 6.75 (m, 1 H), 6.61 - 6.60 (m, 1 H), 4.14 (d, J = 12.8 Hz, 2 H), 3.91 (s, 3 H), 3.88 (s, 3 H), 3.02 (t, J = 12 Hz, 2 H), 2.88 - 2.83 (m, 2 H), 2.43 (d, J= 4.8 Hz, 3 H), 1.80 - 1.77 (m, 2 H), 1.75 - 1.6 (bs, 1 H), 1.49 - 1.45 (m, 2 H) 1.36 - 1.33 (m, , 2 H). |
| 169 | 400.1 | |
| 170 | 380.1 | 8.48 (s, 1 H), 7.18 (s, 1 H), 7.16 (s, 1 H), 6.43 - 6.39 (m, 3 H), 6.20 (s, 2 H), 4.04 - 4.01 (m, 2 H), 3.02 - 2.90 (m, 4 H), 1.78 - 1.74 (m, 2 H), 1.66 (m, 1 H), 1.48 - 1.43 (m, 2 H), 1.37 - 1.31 (m, 2 H). |
| 171 | 346.1 | |
| 172 | 395 | |
| 173 | 392.1 | 9.36 (bs, 1 H), 7.14 (d, J= 8.4 Hz, 2 H), 7.06 (d, J= 8.4 Hz, 2 H), 6.98 (bs, 2 H), 6.81 (s, 1 H), 6.60 (s, 1 H), 5.05 (s, 2 H), 3.67 (s, 3 H), 3.58 (s, 3 H), 2.80 (t, J = 6.8 Hz, 2 H), 2.59 (t, J = 7.6 Hz, 2 H). |
| 174 | 428.1 | 13.42 (bs, 1 H), 9.85 (bs, 1 H), 8.24 (d, J = 6.8 Hz, 1 H), 7.89 (s, 1 H), 7.24 (s, 1 H), 7.08 (s, 4 H), 6.55 (d, J= 6.8 Hz, 1 H), 3.93 (d, J =1.2 Hz, 6 H). |
| 175 | 400.1 | |
| 176 | 377.0 | |
| 177 | 477.2 | |
| 178 | 409.1 | |
| 179 | 391.1 | |
| 180 | 389.3 | |
| 181 | 403.3 | |
| 182 | 375.1 | 8.94(bs, 1 H), 8.42(d, J=6.4 Hz, 1 H), 7.81-7.69(m, 3 H), 7.59-7.49(m, 2H), 7.31-7.26(m, 2 H), 7.12(m, 1 H), 4.86(d, J=5.6 Hz, 2 H), 3.96(s, 3 H), 3.89(m, 3 H). |
| 183 | 472.2 | |
| 184 | 384.1 | |
| 185 | 400.01 | 8.52 (s, 1 H), 7.88 (s, 1 H), 7.26 (s, 1 H), 6.44 - 6.42 (m, 3 H), 4.23 (d, J= 12.8 Hz, 2 H), 3.96 (s, 3 H), 3.08 (t, J= 12.4 Hz, 2 H), 2.95 - 2.90 (m, 2 H), 1.80 -1.71 (m, 3H), 1.49 - 1.38 (m, 2H), 1.37 - 1.29 (m, 2H). |
| 186 | 362.0 | 7.52 (m, 4 H), 7.41 (d, J = 3.2 Hz, 1 H), 7.14 - 7.10 (m, 2 H), 7.02 (s, 1 H), 6.65 (s, 2 H), 6.53 (d, J = 3.2 Hz, 1 H), 4.13 (d, J = 6.8 Hz, 2 H), 3.77 (s, 3 H), |
| | | 3.73 (s, 3 H). |
| 187 | 342.3 | 8.48 (d, J = 4.8 Hz, 1 H), 7.61 (s, 1 H), 7.29 (s, 1 H), 7.17 (s, 1 H), 6.90 (d, J = 4.8 Hz, 1 H), 3.90 (s, 6 H), 3.49 (s, 2 H), 3.20 (t, J= 6.8 Hz, 2 H), 2.95 (s, 2 H), 2.03 (t, J = 7.2 Hz, 4 H), 1.56 (d, J = 13.2 Hz, 2 H). |
| 188 | 401.9 | |
| 189 | 367.3 | 8.90 (s, 1 H), 8.50 (s, 1 H), 7.07 (s, 1 H), 6.45 - 6.42 (m, 3 H), 4.36 - 4.32 (m, 2 H), 3.95 (s, 3 H), 3.17 - 3.11 (m, 2 H), 2.93 - 2.91 (m, 2 H), 1.81 - 1.78 (m, 3 H), 1.46 - 1.45 (m, 2 H), 1.31 - 1.28 (m, 2 H). |
| 190 | 390 | |
| 191 | 390.1 | |
| 192 | 378.3 | |
| 193 | 344.3 | 8.41-8.40(m, 2 H), 7.35-7.29(m, 3 H), 7.09(d, J =4.8 Hz, 1 H), 6.92(s, 2H), 4.27(s, 2 H), 3.46(s, 3 H), 3.32-3.28(m, 2 H), 2.98(t, J=5.2 Hz, 2 H). |
| 194 | 420.30 | 8.62(s, 1 H), 7.90(d, J=9.6 Hz, 1 H), 7.93-7.89(m, 2 H), 6.50-6.40(m, 3 H), 4.28-4.25(m, 2 H), 3.20-3.14(m, 2 H), 2.93-2.92(m, 2 H), 1.81-1.74(m, 3 H), 1.46-1.45(m, 2 H), 1.33-1.30(m, 2 H). |
| 195 | 385.1 | |
| 196 | 355.0 | |
| 197 | 412.0 | |
| 198 | 366.3 | 8.50 (s, 1 H), 7.83 (d, J = 8.8 Hz, 1 H), 7.18 -7.08 (m, 2 H), 6.44-6.40 (m, 3 H), 4.25 - 4.19 (m, 2 H), 3.89 (s, 3 H), 3.11 - 3.05 (m, 2 H), 2.95 - 2.90 (m, 2 H), 1.81 - 1.65 (m, 3 H), 1.50 - 1.43 (m, 2 H), 1.36 - 1.21 (m, 2 H) |
| 199 | 366.0 | |
| 200 | 438.2 | |
| 201 | 394.0 | |
| 202 | 379-3 | |
| 203 | 435.3 | |
| 204 | 366.3 | 9.02 (s, 1 H), 8.62 (d, J = 5.2 Hz, 1 H), 7.04 (s, 1 H), 6.78 (d, J = 4.8 Hz, 1H), 6.45(m, 3 H), 3.95 (s, 3 H), 3.68 - 3.65 (m, 2 H), 2.95 - 2.90 (m, 4 H), 1.83 -1.80 (m, 2 H), 1.64 (m, 1 H), 1.52 - 1.40 (m, 4 H). |
| 205 | 424 | |
| 206 | 384.3 | |
| 207 | 354.0 | |
| 208 | 317.2 | |
| 209 | 420.3 | 8.58(s, 1 H), 8.08(d, J=9.2 Hz, 1 H), 7.63(s, 1 H), 7.46(d, J=8.8 Hz, 1 H), 6.44-6.41(m, 3 H), 4.32(d, J =13.2 Hz, 2 H), 3.16(t, J=12.4 Hz, 2 H), 2.93(q, J =6.4 Hz, 2 H), 1.86-1.74(m, 3 H), 1.49-1.43(m, 2 H), 1.35-1.27(m, 2 H). |
| 210 | 377.3 | |
| 211 | 401 | |
| 212 | 421 | |
| 213 | 368.3 | 8.48(d, J=5.2 Hz, 1 H), 7.29(d, J=10.8 Hz, 1 H), 6.49(d, J =5.2 Hz, 1 H), 6.68(d, J=6.8 Hz, 2 H), 6.52(s, 2 H), 5.15-1.05(m, 1 H), 3.89(s, 3H), 3.88-3.83(m, 4 H), 2.31-1.99(m, 4 H), 1.89-1.74(m, 1 H), 1.66-1.59(m, 1 H). |
| 214 | 365.3 | |
| 215 | 379.3 | 8.45 (s, 1 H), 7.25 (s, 1 H), 7.19 ( s, 1 H), 6.87 (d, J = 5.2 Hz, 1 H), 6.41-6.47 (m, 3 H), 6.16 (s, 2 H), 3.37 (d, J = 12.0 Hz, 2 H), 2.92-3.13 (m, 2 H), 2.65-2.74(m, 2 H), 1.82 (t, 2 H), 1.40-1.56 (m, 5 H). |
| 216 | 391.3 | 9.06 (s, 1 H), 8.61 (s, 1 H), 7.08 (s, 1 H), 6.47 (bs, 3 H), 3.98 (s, 3 H), 3.89 -3.86 (m, 2 H), 3.43 - 3.37 (m, 2 H), 2.96 - 2.93 (m, 2 H), 1.87 - 1.84 (m, 2 H), 1.74 (m, 1 H), 1.53 - 1.48 (m, 2 H), 1.45 - 1.37 (m, 2 H). |
| 217 | 377.3 | |
| 218 | 370.4 | 8.26 (s, 1 H), 6.44 - 6.41 (m, 3 H), 3.61 (d, J= 12.8 Hz, 2 H), 3.42 (s, 3H), 3.28 (s, 3 H), 2.92 (q, J= 6.8 Hz, 2 H), 2.82 (t, J= 12.4 Hz, 2 H), 1.75 (d, J = 12 Hz, 2 H), 1.62 - 1.52 (m, 1 H), 1.45 (q, J= 7.2 Hz, 2 H), 1.32 - 1.22 (m, 2 H); |
| 219 | 361.1 | |
| 220 | 383 | 8.60 (d, J = 4.8 Hz, 1 H), 7.59 (d, J = 12.8 Hz, 1 H), 7.51 (d, J= 8.8 Hz, 1 H), 6.91(d, J = 7.2 Hz, 1 H), 6.47-6.45 (m, 3 H), 3.99 (s, 3 H), 3.45 (d, J = 11.6 Hz, 2 H), 3.0-2.95 (m, 2 H); 2.79 (t, J= 10.8 Hz, 2 H), 1.83 (d, J= 12.0 Hz, 2 H); 1.60-1.55(m, 1 H); 1.54-1.46 (m, 4 H) |
| 221 | 404.3 | 8.57 (s, 1 H), 7.34 (s, 1 H), 7.26 (s, 1 H), 6.47 (bs, 3 H), 6.24 (s, 2 H), 3.62 -3.59 (m, 2 H), 3.36 - 3.33 (m, 2 H), 2.96 - 2.94 (m, 2 H), 1.83 - 1.80 (m, 2H), 1.67 (m, 1 H), 1.52 - 1.50 (m, 2 H), 1.46 - 1.37 (m, 2 H). |
| 222 | 345.4 | |
| 223 | 421.1 | |
| 224 | 348 | |
| 225 | 374.1 | 8.69 (d, J = 4.8 Hz, 1 H), 7.46 (s, 1 H), 7.39 - 7.31(m, 3 H), 6.91 (d, J= 5.8 Hz, 3 H), 4.28 (bs, 2 H), 3.93 (s, 3 H), 3.71 (s, 3 H), 3.33 - 3.28 (m, 2 H), 3.01 (m, 2 H) |
| 226 | 345.3 | |
| 227 | 348.3 | |
| 228 | 415.9 | |
| 229 | 418.3 | 8.75 (s, 1 H), 7.46 (s, 1 H), 7.39 - 7.31(m, 3 H), 6.91 (d, J= 5.8 Hz, 3 H), 4.28 (bs, 2 H), 3.93(s, 3 H), 3.71 (s, 3 H), 3.33 - 3.28 (m, 2 H), 3.01 (m, 2 H) |
| 230 | 345.0 | 8.7 (bs, 1 H), 8.34 (s, 1 H), 8.19 (d, J= 8.4 Hz, 1 H), 7.74 (d, J= 6.8 Hz, 2 H), 7.48 (d, J= 7.2 Hz, 2 H), 7.24 (s, 2 H), 7.13 (d, J = 8.8 Hz, 1 H), 7.08 (s, 1 H), 4.79 (d, J = 5.2 Hz, 2 H), 3.87 (s, 3 H). |
| 231 | 346.0 | 9.45 (bs, 1 H), 8.94 (s, 1 H), 8.66 (d, J= 9.2 Hz, 1 H), 8.34 (d, J= 6 Hz, 1H), 7.24 - 7.19 (m, 4 H), 7.05 - 6.97 (m, 3 H), 6.63 (d, J= 5.2 Hz, 1 H), 3.96 (s, 3 H). |
| 232 | 341.0 | |
| 233 | 431.2 | |
| 234 | 390.0 | 9.43 (bs, 1 H), 8.41 (s, 1 H), 7.82 (s, 1 H), 7.72 - 7.70 (m, 3 H), 7.34 (d, J = 8 Hz, 2 H), 7.16 (s, 1 H), 6.92 (bs, 1 H), 6.59 (s, 2 H), 4.06 (d, J= 6 Hz, 2 H), 3.94 (s, 3 H), 3.91 (s, 3 H) |
| 235 | 370.0 | 9.33 (s, 1 H), 9.13 (s, 1 H), 7.63 (d, J= 8.4 Hz, 2 H), 7.57 (d, J= 8.4 Hz, 2 H), 7.19 (t, J= 6.4 Hz, 1 H), 6.77 (s, 1 H), 6.67 (s, 1 H), 4.22 (d, J= 6 Hz, 2 H), 3.93 (s, 3 H). |
| 236 | 392 | |
| 237 | 395.0 | 9.05 (bs, 1 H), 8.12 - 8.09 (m, 1 H), 7.64 - 7.62 (m, 1 H), 7.43 - 7.40 (m, 3 H), 6.97 - 6.94 (m, 3 H), 4.35 - 4.23 (m, 2 H), 3.74 (s, 3 H), 3.36 - 3.33 (m, 2 H), 3.05 (bs, 2 H). |
| 238 | 402.4 | |
| 239 | 362.3 | 8.84 (d, J= 4.4 Hz, 1 H), 7.66 (d, J= 8.4 Hz, 1 H), 7.54 (d, J= 7.6 Hz, 2 H), 7.50 - 7.46 (m, 3 H), 7.34 (d, J= 4.4 Hz, 1 H), 7.17 - 7.12 (m, 1 H), 6.65 (s, 2 H), 4.18 (d, J= 6.0 Hz, 2 H), 4.02 (s, 3 H); |
| 240 | 369.0 | |
| 241 | 399.0 | |
| 242 | 317.0 | |
| 243 | 402 | |
| 244 | 369 | |
| 245 | 391.1 | 9.07 (s, 1 H), 8.66 (s, 1 H), 7.06 (s, 1 H), 6.46 (bs, 3 H), 4.03 - 4.00 (m, 2 H), 3.97 (s, 3 H), 3.52 (t, J= 12.4 Hz, 2 H), 2.95 - 2.93 (m, 2 H), 1.85 - 1.82 (m, 2 H), 1.75 (m, 1 H), 1.50 - 1.37 (m, 4 H). |
| 246 | 430.0 | |
| 247 | 346.0 | |
| 248 | 402.3 | |
| 249 | 395.3 | |
| 250 | 366.3 | |
| 251 | 419.4 | |
| 252 | 304.2 | |
| 253 | 404.3 | 9.33(s, 1 H), 8.28(s, 1 H), 8.07(bs, 1 H), 7.73(s, 1 H), 7.30(d, J=8.4 Hz, 2H), 7.09-7.05(m, 3 H), 6.96(s, 2 H), 5.45(t, J=7.2 Hz, 1 H), 3.90(s, 3 H), 3.87(s, 3 H), 1.54(d, J =6.8 Hz, 3 H) |
| 254 | 404.3 | 9.32(s, 1 H), 8.25(s, 1 H), 7.99(bs, 1 H), 7.71(s, 1 H), 7.30(d, J=8.0 Hz, 2 H), 7.09-7.05(m, 3 H), 6.96(s, 2 H), 5.53(t, J=7.2 Hz, 1 H), 3.90(s, 3 H), 3.86(s, 3 H), 1.54(d, J =6.8 Hz, 3 H) |
| 255 | 403.1 | 9.8 (bs, 1 H), 8.32 (d, J= 6.8 Hz, 1 H), 7.88 (s, 1 H), 7.47 (d, J= 8.0 Hz, 2H), 7.40 (d, J= 8.0 Hz, 2 H), 7.30 (s, 1 H), 6.88 (s, 2 H), 6.76 (d, J = 6.4 Hz, 1H), 4.12 (s, 2 H), 3.95 (s, 6 H), 2.56 (s, 3 H). |
| 256 | 403.3 | 9.06 (s, 1 H), 8.61 (s, 1 H), 7.11 (s, 1 H), 6.73(bs, 2 H), 3.98 (s, 3 H), 3.65 -3.63(m, 4 H), 3.23 (t, J= 7.2 Hz, 2 H), 3.11 (s, 2 H), 1.86 - 1.74 (m, 6 H). |
| 257 | 393.9 | 9.32 (s, 1 H), 9.13 (s, 1 H), 7.44 -7.42 (m, 3 H), 6.94 (bs, 2 H), 6.82 (s, 1 H), 4.32 - 4.27 (m, 2 H), 3.94 (s, 3 H), 3.34 (m, 2 H), 3.05 (m, 2 H). |
| 258 | 412.0 | 9.60 (bs, 1 H), 8.48 (d, J= 5.2 Hz, 1 H), 7.56 - 7.48 (m, 3 H), 7.39 (s, 1 H), 7.29 (s, 2 H), 6.51 (d, J= 5.2 Hz, 1 H), 3.93 (s, 3 H), 3.92 (s, 3 H) |
| 259 | 363.1 | |
| 260 | 434.0 | 8.75 (s, 1 H), 7.46 (s, 1 H), 7.38 (d, J= 7.6 Hz, 1 H), 7.23 - 7.20 (m, 2 H), 6.92 (s, 2 H), 6.71 (s, 1 H), 4.27 (q, J = 15.6 Hz, 2 H), 3.94 (s, 3 H), 3.65 (s, 3 H), 3.40 - 3.36 (m, 2 H), 3.07 - 2.98 (m, 2 H); |
| 261 | 415.1 | 9.75 (s, 1 H), 8.46 (d, J= 4.8 Hz, 1 H), 7.47 (s, 1 H), 7.37 (s, 1 H), 7.29 (d, J= 8.8 Hz, 2 H), 7.21 (d, J= 8.8 Hz, 2 H), 6.47 (s, 1 H), 3.91 (d, J= 8.0 Hz, 7 H), 2.33 - 2.31 (m, 2 H), 2.18 - 2.16 (m, 2 H), 1.94 - 1.86 (m, 2 H). |
| 262 | 431.3 | |
| 263 | 388.0 | |
| 264 | 345.1 | |
| 265 | 413.3 | 8.67 (d, J= 5.2 Hz, 1 H), 8.02 (d, J= 9.2 Hz, 1 H), 7.61 - 7.60 (m, 1 H), 7.45 (t, J= 74 Hz, 1 H), 7.40 (d, J= 8.0 Hz, 2 H), 7.37 - 7.34 (m, 1 H), 6.96 (d, J= 4.8 Hz, 1 H), 6.72 (s, 2 H), 3.22 (t, J= 7.2 Hz, 2 H), 3.17 - 3.09 (m, 2 H), 3.06 (s, 2 H), 1.82 - 1.74 (m, 6 H). |
| 266 | 363 | |
| 267 | 359.3 | 9.36 (bs, 1 H), 8.20 (d, J= 5.2 Hz, 1 H), 8.16 (d, J= 9.2 Hz, 1 H), 7.73 (m, 1 H), 7.26 (d, J= 8.4 Hz, 2H), 7.14 - 7.04 (m, 4 H), 6.98 (bs, 2 H), 6.21 (d, J = 5.6 Hz, 1 H), 4.42 (d, J = 5.6 Hz, 2 H), 3.84 (s,3 H). |
| 268 | 363.9 | 9.59 (bs, 1 H), 8.63 (d, J= 5.6 Hz, 1 H), 8.09 (d, J= 9.2 Hz, 1 H), 7.64 (t, J = 8.4 Hz, 1 H), 7.27 (d,J= 8.8 Hz, 2 H), 7.21 (d,J= 8.8 Hz, 2 H), 7.11 (bs, 2 H), 6.46 (d, J = 4.8 Hz, 1 H), 4.01 (s,3 H). |
| 269 | 362.9 | 9.46 (s, 1 H), 9.02 (bs, 1 H), 8.33 (d, J = 4.8 Hz, 1 H), 8.18 (d, J= 8.8 Hz, 1H), 7.50(t, J = 7.6 Hz, 1 H), 7.23 - 7.22 (m, 4 H), 7.06 (bs, 2 H), 6.59 (d, J = 5.6 Hz, 1 H), 3.98 (s, 3 H). |
| 270 | 358.9 | 9.82 (s, 1 H), 9.28 (s, 1 H), 8.18 (d, J= 8.8 Hz, 2 H), 7.40 (d, J= 3.2 Hz, 2H), 7.30 (d, J= 8.8 Hz, 2 H), 7.24 (s, 2 H), 3.98 (s, 3 H), 3.96 (s, 3 H); |
| 271 | 412.3 | |
| 272 | 382.3 | 8.30(s, 1 H), 7.58(s, 1 H), 7.50(d, J =6.8 Hz, 2 H), 7.05(s, 1 H), 6.5-6.46(m, 2 H), 6.40(s,lH), 4.15-4.0(m, 1 H), 3.88(s, 3 H) ,3.86(s, 3 H), 3.07(s, 1 H), 2.05-1.978(m, 4 H), 1.44-1.28(m, 4 H). |
| 273 | 377.3 | |
| 274 | 327.3 (M-H) | |
| 275 | 323.1 | |
| 276 | 316.1 | |
| 277 | 290.1 | |
| 278 | 375.3 | 9.32 (s, 1 H), 8.22 (d, J= 8 Hz, 2 H), 7.52 (d, J= 8 Hz, 2 H), 7.43 (d, J= 9.6 |
| | | Hz, 2 H) 7.13 - 7.10 (m, 1 H), 6.64 (s, 2 H), 4.15 (d, J= 6 Hz, 2 H), 3.98 (s, 3 H), 3.97(s, 3 H). |
| 279 | 374.4 | 9.30 (s, 1 H), 8.32 (d, J= 7.6 Hz, 1 H), 7.88 (s, 1 H), 8.19 (d, J= 8.0 Hz, 1H), 7.31- 7.29 (m, 2 H), 7.12 - 7.03 (m, 4 H), 6.95 (s, 2 H), 5.54 - 5.48 (m, 1H), 3.86 (s, 3 H), 1.51 (d, J = 6.8 Hz, 3 H). |
| 280 | 376.9 | |
| 281 | 401.1 | 8.96 (s, 1 H), 8.53 (d, J = 4.8 Hz, 1 H ), 7.35 (s, 1 H), 7.15 - 7.10 (m, 3H), 7.10 (s, 1 H), 6.82 - 6.81 (m, 3 H), 4.03 (t, J = 8 Hz, 2 H), 3.92 (s, 3 H), 3.74 (s, 3 H), 3.14 (t, J= 8.0 Hz, 2 H) |
| 282 | 363.9 | |
| 283 | 440.4 | 8.31(s, 1 H), 7.36-7.34(m, 2 H), 7.18-7.16(m, 2 H), 6.90(s, 2 H), 6.63(s, 1H), 4.31-4.22(m, 2 H), 3.89(s, 3 H), 3.60(s, 3 H), 3.36-3.30(m, 2 H), 3.06-3.01(m, 2 H), 1.68-1.61(m, 1 H), 0.81-0.77(m, 4 H). |
| 284 | 339.3 | |
| 285 | 415.4 | |
| 286 | 375.9 | 9.50 (s, 1 H), 9.07 (bs, 1 H), 8.29 (s, 1 H), 7.81 (d, J= 8.8 Hz, 2 H), 7.21 (s, 1 H), 7.15 (d, J= 8.8 Hz, 2 H), 7.11 (s, 1 H), 6.91 (bs, 2 H), 3.90(s, 3 H), 3.85 (s, 3 H). |
| 287 | 418.0 | 9.25 (s, 1 H), 8.07 (s, 1 H), 7.76 (s, 1 H), 7.55 (s, 1 H), 7.12 (d, J= 8.8 Hz, 2 H), 7.03 - 6.99 (m, 3 H), 6.95 (s, 2 H), 3.94 (s, 3 H), 3.86 (s, 3 H), 1.79 (s, 6 H). |
| 288 | 366.3 | 8.59 (s, 1 H), 7.88 - 7.84 (m, 1 H), 7.74 - 7.69 (m, 1 H), 7.65 - 7.62 (m, 1H), 6.71 (s, 2 H), 3.73 - 3.62 (m, 4 H), 3.21 (t, J= 7.2 Hz, 2 H), 3.05 (s, 2 H), 1.79 (t, J= 7.2, 2 H), 1.75 - 1.65 (m, 4 H). |
| 289 | 384.4 | |
| 290 | 439.1 | 9.0 (s, 1 H), 7.59 - 7.57 (m, 2 H), 7.54 - 7.51 (m, 3 H), 7.0 (s, 2 H), 6.87 (s, 1 H), 4.39 (s, 2 H), 3.99 (s, 3 H), 3.68 (s, 3 H), 2.40 - 2.36 (m, 1 H), 0.56 - 0.51 (m, 4 H). |
| 291 | 415.0 | |
| 292 | 341.0 | |
| 293 | 344.1 | 8.20 - 8.16 (m, 2 H), 7.84 (t, J= 6 Hz, 1 H), 7.75 (d, J= 8.4 Hz, 2 H), 7.52 (d, J= 8.4 Hz, 2 H), 7.24 ( s, 2 H), 7.15 - 7.14 (m, 1 H), 7.10 - 7.07 (m, 1 H), 6.15 (d, J= 5.2 Hz, 1 H), 4.58 (d, J= 5.6 Hz, 2 H), 3.85 (s, 3 H). |
| 294 | 409.1 | ¹HNMR (400 MHz, DMSO-d₆): δ 9.06 (s, 1 H), 8.11 (d, J= 9.2 Hz, 1 H), 7.64 - 7.52 (m, 5 H), 7.01 - 6.91 (m, 3 H), 4.4 (s, 2 H), 3.72 (s, 3 H), 2.48 - 2.38 (m, 1 H), 0.55 - 0.52 (m, 4 H). |
| 295 | 401.4 | |
| 296 | 332.0 | |
| 297 | 364.0 | 9.14 (bs, 1 H), 8.62 (d, J= 4.8 Hz, 1 H), 8.14 (d, J= 9.2 Hz, 1 H), 7.54 (t, J= 8.8 Hz, 1 H), 7.39 (s, 1 H), 7.27 (d, J= 10.4 Hz, 2 H), 7.09 - 7.06 (m, 3 H), 6.54 (d, J = 4.8 Hz, 1 H), 3.92 (s, 3 H). |
| 298 | 334.0 | 9.55(s, 1 H), 8.68(d, J=5.6 Hz, 1 H), 8.40-8.36(m, 2 H), 7.75-7.73(m, 1 H), 7.29-7.21(m, 4 H), 7.12(s, 2 H), 6.53(d, J=5.2 Hz, 1 H). |
| 299 | 374.0 | |
| 300 | 360.0 | 9.19 (s, 1 H), 8.77 (s, 1 H), 7.69 (d,J= 8.8 Hz, 1 H), 7.42 (s, 1 H),7.18 (d, J= 9.2 Hz, 1 H), 7.09 (d, J= 8.8 Hz, 2 H), 6.926 (s, 2 H), 6.74 (d, J= 8.8 Hz, 2 H), 3.89 (s, 3 H), 2.17 (s, 3 H) |
| 301 | 393.0 | |
| 302 | 361.9 | 9.96 (s, 1 H), 8.51 (d, J= 4.8 Hz, 1 H), 8.05 (d, J= 9.2 Hz, 1 H), 7.54 (d, J= 8.8 Hz, 2 H), 7.32 - 7.28 (m, 6 H), 6.54 (d, J= 4.8 Hz, 1 H), 3.91 (s, 3 H); |
| 303 | 361.0 | |
| 304 | 333.0 | 8.97 (s, 1 H), 8.40 (m, J= 13.6 Hz, 2 H), 7.74 (d, J= 8.0 Hz, 2 H), 7.51-7.41 (m, 4 H), 7.25 (s, 2 H), 4.81 (s, 2 H). |
| 305 | 370.0 | 8.82 (d, J= 4.4 Hz, 1 H), 7.77 (d, J= 9.6 Hz, 1 H), 7.45 (d, J= 2.8 Hz, 1 H), |
| | | 7.35 - 7.27 (m, 3 H), 7.26 - 7.21 (m, 2 H), 6.91 (s, 2 H), 4.28(s, 2 H), 3.92(s, 3 H), 3.34 - 3.27 (m, 2 H), 3.15 - 2.99(m, 2 H). |
| 306 | 345.0 | |
| 307 | 381 | |
| 308 | 359.9 | |
| 309 | 436 | 8.59 (d, J= 5.2 Hz, 1 H), 8.11(d, J= 8.8 Hz, 2 H), 7.40 - 7.26 (m, 6 H), 7.24 -7.15 (m, 6 H), 6.38 (d, J = 4.8 Hz, 1 H), 4.75 (s, 2 H), 3.90 (s, 3 H) |
| 310 | 386.1 | 8.62 (d, J = 4.8 Hz, 1 H), 8.16 (d, J= 9.2 Hz, 1 H), 7.42 - 7.40 (m, 3 H), 7.29 -7.23 (m, 3 H), 7.11 (s, 2 H), 6.52 (d, J = 5.2 Hz, 1 H), 3.92 (s, 3 H), 2.97(bs, 1H), 0.77 - 0.76 (m, 2 H), 0.59 (bs, 2 H). |
| 311 | 358.0 | |
| 312 | 345.0 | 8.61 (d, J = 5.2 Hz, 1 H), 8.16 (d, J = 8.8 Hz, 1 H), 7.48 (d, J= 8.0 Hz, 2 H), 7.40 (s, 1 H), 7.26 (d, J = 8.4 Hz, 3 H), 6.82 (s, 2 H), 6.53 (d, J = 5.6 Hz, 1 H), 4.31 (s, 2 H), 3.92 (s, 3 H); |
| 313 | 399.4 | |
| 314 | 375.3 | |
| 315 | 371.0 | 9.53 (s, 1 H), 8.63 (d, J= 4.8 Hz, 1 H), 8.15 (d, J= 9.2 Hz, 1 H), 7.81 (s, 1H), 7.82 - 7.60 (m, 2 H), 7.41 (d, J= 2.8 Hz, 1 H), 7.30 - 7.27 (m, 1 H), 7.22 (s, 2 H), 6.55 (d, J = Hz, 1 H), 3.92 (s, 3 H). |
| 316 | 413.0 | 9.14 (s, 1 H), 7.76 (d, J= 8.4 Hz, 1 H), 7.41 - 7.36 (m, 4 H), 6.92 (s, 2 H), 4.36 - 4.32 (m, 2 H), 4.06 (s, 3 H), 3.33 - 3.27 (m, 2 H), 3.05 - 3.03 (m, 2 H). |
| 317 | 414.1 | 9.94(s, 1 H), 8.60(d, J=5.2 Hz, 1 H), 8.13(d,J=9.2 Hz, 1 H), 7.60(s, 1 H), 7.51-7.40(m, 3 H), 7.31(s, 3 H), 6.46(d, J=4.8 Hz, 1 H), 3.92(s, 3 H). |
| 318 | 321.0 | |
| 319 | 386.4 | |
| 320 | 347.0 | 9.65(s, 1 H), 8.67(d, J=5.2 Hz, 1 H), 8.05(s, 1 H), 8.02(d, J=8.8 Hz, 1 H), 7.74-7.72(m, 1 H), 7.40(s, 1 H), 7.29-7.18(m, 4 H), 6.79(d, J =4.8 Hz, 1 H), 3.91(s, 3 H). |
| 321 | 383.9 | 11.64(s, 1 H), 9.47(s, 1 H), 8.02(d, J=5.2 Hz, 1 H), 7.03(s, 1 H), 7.21(d, J=8.8 Hz, 2 H), 7.11-7.062(m, 4 H), 6.32(d, J=5.6 Hz, 1 H), 6.19(s, 1 H). |
| 322 | 347.0 | |
| 323 | 376.3 | |
| 324 | 385.4 | 9.19 (s, 1 H), 7.97 (d, J = 9.2 Hz, 1 H), 7.70 (d, J= 8.4 Hz, 2H), 7.53 (d, J= 8 Hz, 2 H), 7.43 - 7.42 (m, 1 H), 7.34 - 7.31 (m, 1 H), 6.98 (bs, 2 H), 4.38 (s, 2 H), 3.97 (s, 3 H), 2.42 - 2.39 (m, 1 H), 0.59 - 0.58 (m, 4 H). |
| 329 | 379.9 | 9.83 (s, 1 H), 8.57 (d, J= 5.2 Hz, 1 H), 8.21(d, J= 8.8 Hz, 1 H), 7.42 - 7.35 (m, 3 H), 7.31- 7.23 (m, 3 H), 7.21 (d, J= 8.8 Hz, 1 H), 6.31 (d, J = 4.8 Hz, 1 H), 3.92(s, 3 H). |
| 330 | 346.9 | 9.61 (s, 1 H), 9.40 (s, 1 H), 8.71 (d, J= 5.2 Hz, 1 H), 7.29 - 7.24 (m, 4 H), 7.18 (s, 1 H), 7.12 (s, 2 H), 6.40 (d, J= 5.2 Hz, 1 H), 3.99 (s, 3 H). |
| 345 | 358.1 | 8.84 (d, J= 4.4 Hz, 1 H), 7.45 (d, J= 2.0 Hz, 1 H), 7.36 (s, 1 H), 7.32-7.25(m, 2 H), 7.18-7.14 (m, 3 H), 7.07 (t, J= 6.8 Hz, 1 H), 6.63 (s, 2 H), 4.13 (d, J= 6.8 Hz, 2 H), 3.91 (s, 3 H), 1.94 (s, 3 H) |
| 346 | 346.9 | 9.51 (s, 1 H), 8.55 (d, = 5.2 Hz, 1 H), 8.24 (d, J= 9.2 Hz, 1 H), 7.28 - 7.23 (m, 3 H), 7.15 (d, J= 8.8 Hz, 2 H), 7.08 (s, 2 H), 6.81 (d, J= 4.8 Hz, 1 H), 3.93 (s, 3 H). |
| 347 | 326.0 | 12.58 (bs, 1 H), 8.65 (d, J = 4.8 Hz, 1 H), 8.12 (d, J= 9.2 Hz, 1 H), 7.76 (d, J= 8.4 Hz, 1 H), 7.41 (s, 1 H), 7.27 (d, J = 9.2 Hz, 1 H), 7.05 (s, 1 H), 6.77 (d, J= 8.0 Hz, 1 H), 6.67 (d, J= 4.8 Hz, 1 H), 3.92 (s, 3 H), 3.79 (s, 3 H) |
| 348 | 333.0 | 8.22 (s, 1 H), 7.76 (d, J= 8.8 Hz, 1 H), 7.66 (d, J= 8.4 Hz, 2 H), 7.44 (d, J= 7.6 Hz, 2 H), 7.25 (s, 1 H), 7.06 (brs, 1H), 6.95 (d, J= 8.8 Hz, 1 H), 6.62 (s, 2 H), 4.10 (d, J= 6.0 Hz, 2 H), 3.81 (s, 3 H) |
| 349 | 347.0 | 9.54 (bs, 1H), 8.70 (d, J= 2.4 Hz, 1 H), 8.65 (d, J= 5.6 Hz, 1 H), 7.75 (d, J= 3.2 Hz, 1 H), 7.26 (d, J= 9.2 Hz, 2 H), 7.17 (d, J= 8.8 Hz, 2 H), 7.09 (s, 2 H), |
| | | 6.64 (d, J = 5.2 Hz, 1 H), 3.98 (s, 3 H). |
| 350 | 298.1 | 8.63 (d, J = 5.6 Hz, 1 H), 8.13 (d, J = 9.2 Hz, 1 H), 8.00 - 7.95 (m, 3 H), 7.41 -7.35 (m, 2 H), 7.30 - 7.25 (m, 3 H), 6.58 (d, J= 4.8 Hz, 1 H). |
| 351 | 361.0 | 9.60 (s, 1 H), 8.65 (d, J= 4.8 Hz, 1 H), 8.06 (d, J= 9.2 Hz, 1 H), 7.87 (d, J= 2.8 Hz, 1 H), 7.62 (d, J= 2.4 Hz, 1 H), 7.46 (d, J= 8.8 Hz, 1 H), 7.25 (dd, Jl= 8.8 Hz, J 2= 2.4 Hz, 1 H), 7.17 (s, 2 H), 6.72 (d, J= 4.8 Hz, 1 H), 3.91 (s, 3 H), 2.26 (s, 3 H). |
| 352 | 318.0 | 12.22 (bs, 1H), 8.37(d, J= 5.6 Hz, 1 H), 8.07 (d, J= 9.6 Hz, 1 H), 7.90 (d, J= 8.8 Hz, 2 H), 7.42-7.39 (m, 4 H), 6.57-6.52 (m, 2 H). |
| 353 | 371.2 | 10.02 (s, 1 H), 8.65 (d, J = 5.2 Hz, 1 H), 8.18 (d, J= 9.2 Hz, 1 H), 7.63 (s, 1 H), 7.49 - 7.43 (m, 3 H), 7.35 - 7.30 (m, 3 H), 6.51 (d, J= 4.8 Hz, 1 H), 3.93 (s, 3 H), |
| 354 | 345.2 | 8.87 (d, J= 4.4 Hz, 1 H), 8.62 (d, J= 8.6 Hz, 1 H), 7.99 - 7.96 (m, 1 H), 7.70 -7.67 (m, 2 H), 7.49 (d, J= 2.4 Hz, 1 H), 7.34 (d, J= 4.4 Hz, 1 H), 7.26 - 7.10 (m, 2 H), 6.69 (s, 2 H), 4.30(d, J= 6.4 Hz, 2 H), 3.92 (s, 3 H). |
| 355 | 331.0 | 8.60 (d, J = 5.2 Hz, 1 H), 7.97 - 7.90 (m, 3 H), 7.47 - 7.39 (m, 6 H), 6.77 (d, J= 4.8 Hz, 1 H), 3.99 (s, 3 H). |
| 356 | 332.0 | 8.63 (s, 1 H), 8.26 (d, J= 9.6 Hz, 1 H), 7.91 (d, J= 8.8 Hz, 2 H), 7.52 (d, J= 8.8 Hz, 2 H), 7.39 - 7.37 (m, 4 H), 3.96 (s, 3 H). |
| 357 | 372.1 | 8.82 (d, J= 4.4 Hz, 1 H), 7.78 (d,J= 9.2 Hz, 1 H), 7.67 (d,J= 8 Hz, 2 H), 7.45-7.43 (m, 3 H), 7.27 (d, J= 4.4 Hz, 1 H), 7.22 (dd, J1 = 2 Hz, J2 = 2 Hz, 1 H), 7.04 (s, 1 H), 6.45 (s, 2 H), 3.91 (s, 3 H), 1.65 (s, 6 H). |
| 358 | 370.0 | 8.75 (d, J= 3.2 Hz, 1 H), 7.99 (d, J= 9.2, 1 H), 7.44-7.36 (m, 5 H), 7.18 (s, 1 H), 6.91 (s, 2 H), 4.29 (s, 2 H), 3.75 (s, 3 H), 3.32 (bs, 2H), 3.00 (bs, 2 H). |
| 359 | 345.2 | 8.82 (s, 1 H), 7.75 (d, J= 8.8 Hz, 2 H), 7.64 (d, J= 9.2 Hz, 1 H), 7.44 (s, 1 H), 7.25 (s, 2 H), 7.28 - 7.18 (m, 1 H), 6.96 (d, J= 8.8 Hz, 2 H), 3.89 (s, 3 H), 2.18 (s, 3 H). |
| 360 | 379.1 | 9.58 (s, 1 H), 8.51 (s, 1 H), 8.18 (d, J= 8.8 Hz, 1 H), 7.35 (s, 1 H), 7.25 - 7.08 (m, , 4 H), 7.01 (s, 1 H), 6.81 (d, J= 7.6 Hz, 1 H), 6.25 (d, J= 4 Hz, 1 H),3.66 (s, 3 H). |
| 361 | 332.2 | 8.75(d, J = 5.6 Hz, 1 H), 8.51 (d, J= 9.2 Hz, 1 H), 7.92 (d, J= 8.8 Hz, 2H), 7.44-7.39 (m, 4 H), 7.14 (d, J= 8.8 Hz, 1 H), 6.72 (d, J = 5.2 Hz, 1 H), 4.02 (s, 3 H) |
| 362 | 359.1 (M-H)⁺ | 10.20 (bs, 1H), 8.92 (d, J= 6.4 Hz, 1 H), 8.85 (bs, 1H), 8.46 (d, J= 9.2 Hz, 1 H), 7.67 (s, 1 H), 7.57 (d, J= 9.2 Hz, 1 H), 7.37 - 7.35 (m, 4 H), 6.80 (d, J= 6.4 Hz, 1 H), 4.00(s, 3 H). |
| 363 | 377.1 | 8.56 (d, J = 4.8 Hz, 1 H), 7.85 (d, J = 9.2 Hz, 1 H), 7.28 (s, 1 H), 7.14 (d, J= 9.2 Hz, 1 H), 6.82 (d, J = 4.8 Hz, 1 H), 6.71 (s, 2 H), 3.86 (s, 3 H), 3.21 (t, J = 6.8 Hz 2 H), 3.18-3.00 (m, 6 H), 1.85-1.70 (m, 6 H) |
| 364 | 377.1 | 8.52 (d, J = 5.2 Hz, 1 H), 7.83 (d, J = 9.2 Hz, 1 H), 7.34-7.31 (m, 1 H), 7.21 (d, J= 2.8 Hz, 1 H), 6.95 (d, J= 4.8 Hz, 1 H), 6.71 (s, 2 H), 3.88 (s, 3 H), 3.23 (t, J= 12.8 Hz 2 H), 3.18-3.00 (m, 6 H), 1.85-1.72 (m, 6 H), |
| 365 | 371.3 | 8.91 (d, J = 4.4 Hz, 1 H), 8.16 (d, J = 9.2 Hz, 1 H), 7.39 -7.33 (m, 4 H), 7.08 (d, J= 9.2 Hz, 1 H), 6.91 (s, 2 H), 4.28 (s, 2 H), 4.02 (s, 3 H), 3.32 - 3.30 (m, 2 H), 3.01 - 2.98 (m, 2 H). |
| 366 | 370 | 8.82 (d, J= 4 Hz, 1 H), 7.76 (d,J= 9.2, 1 H), 7.45 (s, 1 H), 7.36-7.31 (m, 3 H), 7.26-7.20 (m, 2 H), 6.92 (s, 2 H), 4.29 (s, 2 H), 3.91 (s, 3 H), 3.31-3.30 (m, 2 H), 2.98 (bs, 2H). |
| 367 | 400.1 | 8.68 (d, J= 4.4 Hz, 1H), 7.43 (s, 1H), 7.40-7.33 (m, 3H), 7.22 (d, J= 4.8 Hz, 1H), 7.16 (s, 1H), 6.92 (s, 2H), 4.29 (s, 2H), 3.93 (s, 3H), 3.73 (s, 3H), 3.32 (t, J= 5.6 Hz, 2H), 3.00 (t, J= 5.2 Hz, 2H). |
| 368 | 358.3 | 8.93 (d, J = 4.4 Hz, 1H), 7.75 (d, J = 9.2 Hz, 1H), 7.51 (s, 4H), 7.46 (d, J= |
| | | 2.8 Hz, 1 H), 7.29 (d, J = 4.4 Hz, 1 H), 7.24 - 7.21 (m, 1 H), 6.89 (s, 2 H), 4.18 (s, 2 H), 3.92 (s, 3 H), 2.59 (m, 3 H). |
| 369 | 345.0 | 8.92 (d, J = 4.4 Hz, 1 H), 8.72 (d, J = 2.4 Hz, 1 H), 7.81 (d, J= 2.8 Hz, 1 H), 7.69 (d, J = 8 Hz, 2 H), 7.59 (d, J= 4.4 Hz, 1 H), 7.47 (d, J = 8 Hz, 2 H) 7.11 (bs, 1H), 6.63 (s, 2 H), 4.15 (bs, 2H), 3.99 (s, 3 H). |
| 370 | 333 | ^{I}HNMR (400 MHz, DMSO-d₆): 8 9.77 (s, 1 H), 8.80 (bs, 1H), 7.79 (d, J= 9.6 Hz, 1 H) 7.43(bs, 3 H), 7.32 - 7.21 (m, 6 H). |
| 371 | 384.3 | 8.75 (s, 1 H), 7.38 (d, J= 2.4 Hz, 1 H), 7.33 (d, J= 8 Hz, 1 H), 7.27 (d, J= 9.2 Hz, 1 H), 7.13 - 7.07 (m, 3 H), 6.90 (s, 2 H), 4.25 (s, 2 H), 3.88 (s, 3 H), 3.33 -3.27 (m, 2 H), 3.00 (t, J= 5.6 Hz, 2 H), 2.17 (s, 3 H). |
| 372 | 332.3 | 11.08 (s, 1 H), 7.54 (d, J = 8.8 Hz, 1 H), 7.63 (s, 1 H), 7.51 - 7.49 (m, 2 H), 7.32 (t, J= 7.6 Hz, 1 H), 7.16 (d, J = 7.6 Hz, 1 H), 7.05 - 7.02 (m, 1 H), 6.91 (s, 1 H), 6.72 (d, J= 6.8 Hz, 1 H), 6.59 (s, 2 H) 4.11 (d, J = 6.4 Hz, 2 H), 3.76 (s, 3 H). |
| 373 | 374.3 | 8.82 (d, J= 4 Hz, 1 H), 7.80 (d, J = 9.2 Hz, 1 H), 7.53 (d, J = 7.6 Hz, 1 H), 7.45 (s, 1 H), 7.30 (s, 1 H), 7.22 (d, J = 8.8 Hz 1 H), 7.06 (d, J = 8 Hz 2 H), 6.90 (bs, 1H), 6.60 (s, 2 H), 4.14 (d, J= 6.4 Hz 2 H), 3.91 (s, 3 H), 3.83 (s, 3 H). |
| 374 | 358.3 | 8.81 (d, J = 3.6 Hz, 1 H), 7.74 (d, J = 9.2 Hz, 1 H), 7.51 (d, J= 7.6 Hz, 1 H), 7.44 (s, 1 H), 7.30-7.21 (m, 4 H), 6.96 (bs, 1H), 6.64 (s, 2 H), 4.12 (d, J = 5.2 Hz, 2 H), 3.91 (s, 3 H), 2.36 (s, 3 H) |
| 375 | 389.2 | 9.44 (s, 1 H), 8.54 (d, J= 4.8 Hz, 1 H), 7.99 (d, J= 9.2 Hz, 1 H), 7.33 (s, 1 H), 7.18 (d, J = 8.8 Hz, 1 H), 7.08 (s, 2 H), 6.96 (s, 1 H), 6.84 (d, J= 13.2 Hz, 1 H), 6.73 (t, J= 4.4 Hz, 1 H), 4.47 (bs, 2H), 3.90 (s, 3 H), 3.08 (bs, 2H). |
| 376 | 389.1 | 9.02 (bs, 1H), 8.68 (d, J = 4.8 Hz, 1 H), 7.72 (d, J = 9.6 Hz, 1 H), 7.50 (t, J = 8 Hz, 1 H), 7.18 (d, J = 5.2 Hz, 1 H), 7.14 (s, 1 H), 6.86 (s, 2 H), 6.80 (d,J= 8.4 Hz, 1 H), 6.43 (d, J = 8.4 Hz, 1 H), 4.06 (t, J = 7.6 Hz, 2 H), 3.98 (s, 3 H), 3.13 (t, J = 7.6 Hz, 2 H). |
| 377 | 398.9 | 9.16 (s, 1 H), 7.76 (d, J = 8.4 Hz, 2H), 7.66 (d, J = 9.2 Hz, 1 H), 7.59 (s, 1 H), 7.33 - 7.30 (m, 3 H), 7.08 (d, J = 8.8 Hz, 2 H), 3.95 (s, 3 H) |
| 378 | 385 | 8.75 (s, 1 H), 8.67 (s, 1 H), 7.74 (d, J= 9.2 Hz, 1 H), 7.40 (d, J= 2.0 Hz, 1 H), 7.16 - 7.13 (m, 1 H), 7.07 (s, 1 H), 6.71 (d, J = 18.0 Hz, 3 H), 5.73 (d, J = 8.4 Hz, 1 H), 3.97 - 3.85 (m, 5 H), 3.27 (s, 2 H), 2.20 (s, 3 H). |
| 379 | 389 | 8.70 (d, J = 5.2 Hz, 1 H), 8.57 (s, 1 H), 7.80 (d, J= 8.8 Hz, 1 H), 7.38 (s, 1 H), 7.18 (t, J= 11.6 Hz, 2 H), 7.0 (t, J= 8.0 Hz, 1 H), 6.78 (s, 2 H), 6.14 (d, J = 8.0 Hz, 1 H), 4.10 (t, J= 7.2 Hz, 2 H), 3.91 (s, 3 H), 3.27 - 3.14 (m, 2 H). |
| 380 | 383.0 | ¹HNMR (400 MHz, DMSO-d₆): δ 9.86 (s, 1 H), 8.57 (d, J= 4.4 Hz, 1 H), 8.21 (d, J= 8.8 Hz, 1 H), 7.40 - 7.18 (m, 7 H), 6.30 (d, J= 4.4 Hz, 1 H). |
| 381 | 362.0 | 8.92 (s, 1 H), 7.56 - 7.48 (m, 4 H), 7.43 (d, J= 7.6 Hz, 2 H), 7.27 (m, 1H), 7.15 (m, 1 H), 6.65 (s, 2 H), 4.18 (d, J= 5.8 Hz, 2 H), 3.91 (s, 3 H) |
| 382 | 349 | 8.65 (d, J= 5.2 Hz, 1 H), 8.16 (d, J = 9.2 Hz, 1 H), 7.86 (d, J= 10 Hz, 1H), 7.74 (d, J = 8.4 Hz, 1 H), 7.61 (t, J= 8 Hz, 1 H), 7.54 (s, 2 H), 7.43 (s, 1H), 7.31 (d, J= 8.8 Hz, 1 H), 6.60 (d, J = 4.8 Hz, 1 H), 3.92 (s, 3 H) |
| 383 | 374.1 | 8.82 (d, J= 4.4 Hz, 1 H), 7.44 (s, 1 H), 7.30 (d, J= 9.2 Hz, 1 H), 7.25 (s, 1 H), 7.20 (d,J=4.4 Hz, 1 H), 7.18 - 7.15 (m, 1 H), 7.11 (d,J=8.4 Hz, 1 H), 6.97-6.90 (m, 2 H), 6.44 (s, 2 H), 3.91 (s, 3 H), 3.84 (s, 3 H), 3.72 - 3.65 (m, 2 H) |
| 384 | 347.3 | 8.82 (d, J= 4.0 Hz, 1 H), 7.73 (d, J= 9.2 Hz, 1 H), 7.60 - 7.46 (m, 5 H), 7.30-7.18 (m, 2 H), 7.12 (bs, 1 H), 6.63 (s, 2 H), 4.17 (d, J= 6.4 Hz, 2 H) |
| 385 | 334.0 | 8.9 1 (d, J= 4.8 Hz, 1 H), 8.05 (s, 1 H), 7.76 (d, J= 9.2 Hz, 1 H), 7.52 (s, 2H), 7.41 (d, J= 4.8 Hz, 1 H), 7.34-7.32 (m, 1 H), 6.83 (bs, 1H), 6.60 (s, 2 H), 4.09 (d, J= 5.2 Hz, 2 H), 3.94 (s, 3 H) |
| 386 | 412.3 | 9.13 (s, 1 H), 7.54 - 7.50 (m, 3 H), 7.30 - 7.17 (m, 5 H), 6.66 (s, 2 H), 4.19 (d, J= 5.6 Hz, 2 H), 3.95 (s, 3 H). |
| 387 | 345.0 | 8.89 (d, J= 4 Hz, 1 H), 8.73 (s, 1 H), 8.07 (d, J= 9.2 Hz, 1 H), 7.96 (d, J= 7.2 Hz, 1 H), |
| | | 7.73 (d, J= 7.2 Hz, 1 H), 7.48-7.42 (m, 2 H), 7.27-7.23 (m, 2 H), 6.72 (bs, 2 H), 4.23 (d, J= 5.6 Hz, 2 H), 3.92 (s, 3 H). |
| 388 | 334.0 | 8.67 (d, J = 5.2 Hz, 1 H), 8.09 (d, J= 9.6 Hz, 1 H), 7.9 (d, J= 8.8 Hz, 2H), 7.42 - 7.38 (m, 5 H), 7.29 - 7.26 (m, 1 H), 6.65 (d, J = 5.2 Hz, 1 H). |
| 389 | 349.9 | 8.79(d, J= 4.4 Hz, 1 H), 8.18 (d, J= 9.6 Hz, 1 H), 7.45 (s, 1 H), 7.35(s, 2 H), 7.30-7.28 (m, 2 H), 7.16(d, J = 2.8 Hz, 1 H), 6.70 (bs, 2 H), 4.32 (s, 2 H), 3.92 (s, 3 H) |
| 390 | 377 | 8.82 (bs, 1H), 7.79 (d, J= 8.8 Hz, 1 H), 7.53-7.52 (m, 1 H) 7.44 (s, 1 H), 7.29 (s, 1 H), 7.21-7.22 (m, 1 H), 7.07 (bs, 2 H), 6.90 (bs, 1 H), 6.60 (s, 2 H), 4.14 (bs, 2H), 3.83 (s, 3 H). |
| 391 | 371.3 | 9.02 (s, 1 H), 8.67 (d, J= 4.8 Hz, 1 H), 7.82 (d, J = 9.2 Hz, 1 H), 7.36 (s, 1 H), 7.20-7.11 (m, 3 H), 6.79 (s, 2 H), 6.66 (d, J= 8 Hz 1 H), 6.33 (s, 1 H), 4.02 (t, J = 7.6 Hz, 2 H), 3.90 (s, 3 H), 3.08 (t, J= 8 Hz, 2 H). |
| 392 | 371.42 | 9.19 (s, 1 H), 8.56 (s, 1 H), 8.02 (d, J= 9.6 Hz, 1 H), 7.35 (d, J= 8.8 Hz, 1 H), 7.20 (s, 1 H), 7.13 (s, 2 H), 6.94 (s, 2 H), 6.90 (s, 1 H), 4.38 (t, J= 7.6 Hz, 2H), 3.11 (s, 3 H), 3.10 (t, J= 8.0 Hz, 2 H). |
| 393 | 331.9 | 9.39 (s, 1 H), 8.79 (d, J = 4.8 Hz, 1 H), 7.95(d, J= 8.8 Hz, 2 H), 7.52 (d, J= 8.4 Hz, 2 H), 7.43 (s, 2 H), 7.23 (s, 1 H), 6.57 (d, J= 4.8 Hz, 1 H), 4.0 (s, 3 H). |
| 394 | 361.9 | 8.83 (d, J = 4.8 Hz, 1 H), 7.43-7.36 (m, 5 H), 7.18 (d, J = 4.4 Hz, 1 H), 7.10-7.03 (m, 2 H), 6.63 (s, 2 H), 4.14 (d, J = 6.4 Hz, 2 H), 3.92 (s, 3 H) |
| 395 | 386.9 | 9.15 (s, 1 H), 7.77 (d, J = 8.4 Hz, 1 H), 7.60 (d, J= 7.6 Hz, 2 H), 7.51 (d,J= 7.6 Hz, 2 H), 7.29 (d, J = 12.4 Hz, 1 H), 7.21 - 7.19 (m, 1 H), 6.67 (s, 2 H), 4.21 (d, J= 6.4 Hz, 2 H), 4.06 (s, 3 H). |
| 396 | 330.0 | 9.76 (bs, 1 H), 8.80 (d, J = 4.4 Hz, 1 H), 7.79 (d, J = 9.2 Hz, 1 H), 7.43 (d, J = 8 Hz, 3 H), 7.31 (d, J = 8.4 Hz, 2 H), 7.25 - 7.19 (m, 4 H), 3.91 (s, 3 H). |
| 397 | 359.9 | 9.55 (s, 1 H), 8.09 (d, J= 9.8 Hz, 1 H), 7.32-7.20 (m, 3 H), 7.16 (d, J= 8.8 Hz, 3 H), 7.08 (s, 2 H), 6.28 (s, 1 H), 3.89 (s, 3 H), 2.44(s, 3 H) |
| 398 | 345.9 | 8.83 (d, J= 4.4 Hz, 1 H), 7.74 (d, J= 9.2 Hz, 1 H), 7.53 (d, J= 8 Hz, 2 H), 7.48 - 7.46 (m, 3 H), 7.27 - 7.21 (m, 2 H), 7.11 (bs, 1 H), 6.63 (bs, 2 H), 3.91 (s, 3 H). |
| 399 | 349.1 | 9.05 (bs, 1 H), 7.16-7.08 (m, 3 H), 6.98 (d, J= 8.8 Hz, 2 H), 6.87 (bs, 2H), 6.48-6.45 (m, 1 H), 6.37 (s, 1 H), 5.30 (s, 1 H), 4.23-4.09 (m, 2 H), 3.69 (s, 3 H), 2.06-1.98 (m, 2 H). |
| 400 | 345.9 | 8.52 (s, 1 H), 8.03 (d, J= 12.0 Hz, 1 H), 7.76 (d, J= 8.0 Hz, 2 H), 7.48 (d, J= 7.6 Hz, 2 H), 7.20 (s, 2 H), 7.12 (d, J= 9.2 Hz, 2 H), 5.21 (s, 2 H), 3.88 (s, 3 H). |
| 401 | 364.1 | 9.40 (s, 1 H), 7.14 (d, J= 8.0 Hz, 2 H), 7.06 (d, J= 8.4 Hz, 2 H), 7.0 (s, 2 H), 6.91 (d, J= 8.8 Hz, 1 H), 6.58 (s, 1 H), 6.50 (d, J= 8.8 Hz, 1 H), 5.01 (s, 2 H), 4.71 (s, 2 H), 3.64 (s, 3 H). |
| 402 | 355.9 | 8.83 (s, 1 H), 7.73 (d, J= 9.2 Hz, 1 H), 7.50 - 7.40 (m, 4 H), 7.27 - 7.20 (m, 2 H), 6.93 (s, 2 H), 4.58 (s, 4 H), 3.91 (s, 3 H) |
| 403 | 358.0 | 8.82 (d, J= 3.6 Hz, 1 H), 7.76 (d, J= 9.2 Hz ,1 H), 7.43 - 7.40 (m, 5 H), 7.27 (d J = 4.4 Hz, 1 H), 7.22 (d, J = 9.6 Hz, 1 H), 6.59 (bs, 1 H), 6.54 (bs, 2 H), 3.91(s, 3 H), 3.17 (m, 2 H), 2.88 (t, J= 7.6 Hz , 2 H). |
| 404 | 372.0 | 8.57 (d, J= 5.2 Hz, 1 H), 8.16 (d, J= 9.2 Hz, 1 H), 7.38-7.24 (m, 5 H), 7.14 (s, 1 H), 7.05 (d, J= 8.4 Hz, 1 H), 6.43 (d, J= 5.2 Hz, 1 H), 3.91 (s, 3 H), 3.86 (t, J= 8.4 Hz, 2 H), 3.08 (t, J= 8.4 Hz 2 H). |
| 405 | 350.1 | 7.25 (d, J= 8 Hz, 2 H), 7.06 (d, J= 8.4 Hz, 2 H), 6.91 (t, J= 6.4 Hz, 1 H), 6.75 (d, J= 8.8 Hz, 1 H), 6.55 (s, 2 H), 6.43 (d, J= 2.4 Hz, 1 H), 6.35 (dd, J1 = 2.8 Hz, J2 = 2.8 Hz, 1 H), 4.13 (t, J= 4 Hz, 2 H), 3.99 (d, J= 6.4 Hz, 2 H), 3.65 (s, 3 H), 3.60 (t, J= 4.4 Hz, 2 H). |
| 406 | 329.0 | 8.83 (d, J= 4.4 Hz, 1 H), 7.77 (d, J= 9.2 Hz, 1 H), 7.55-7.50 (m, 4 H), 7.46 (d, J= 2.0 Hz, 1 H), 7.28-7.21 (m, 2 H), 6.89 (s, 2 H), 4.36 (s, 2 H), 3.92 (s, 3 H) |
| 407 | 414.0 | 9.32 (bs, 1 H), 9.10 (s, 1 H), 7.66 (d, J= 9.2 Hz, 1 H), 7.54 (s, 1 H), 7.27 (d, J = 9.2 Hz, 1 H), 7.09 (d, J= 8.4 Hz, 2 H), 6.99 (bs, 2 H), 6.84 (d, J= 8.4 Hz, |
| | | 2 H), 3.93 (s, 3 H) |
| 408 | 438.0 | 9.13 (s, 1 H), 7.54 (s, 1 H), 7.34 - 7.29 (m, 3 H), 7.17 - 7.13 (m, 2 H), 6.91 (s, 2 H), 4.28 - 4.17 (m, 2 H), 3.95 (s, 3 H), 3.34 - 3.27 (m, 2 H), 3.01 (bs, 2 H) |
| 409 | 388.0 | 8.86 (d, J= 4.4 Hz, 1 H), 7.54-7.46 (m, 2 H), 7.33-7.29 (m, 2 H), 7.23 (d, J= 9.6 Hz, 2 H), 6.92 (s, 2 H), 4.24 (s, 2 H), 3.92 (s, 3 H), 3.31-3.28 (m, 2 H), 3.03-2.98 (m, 2 H). |
| 410 | 388.0 | 8.86 (d, J = 4.4 Hz, 1 H), 7.52 (d, J = 8 Hz, 1 H), 7.46 (d, J = 1.6 Hz, 1H), 7.32-7.28 (m, 2 H), 7.24-7.20 (m, 2 H), 7.00 (s, 2 H), 4.27 (s, 2 H), 3.92 (s, 3 H), 3.37-3.31 (m, 2 H), 3.05-3.01 (m, 2 H). |
| 411 | 347.0 | 9.59 (s, 1 H), 9.13 (s, 1 H), 8.60(d, J=4.8 Hz, 1 H), 7.364(s, 1 H), 7.291-7.212 (q, 4 H), 7.11(s, 2 H), 6.64(d, J=4.8 Hz, 1 H), 3.99(s, 3 H) |
| 412 | 389.0 | 8.96 (s, 1 H), 8.60 (d, J=4.8 Hz, 1 H), 7.22 (s, 1 H), 7.12 (s, 2 H), 6.96 (d, J=13.2 Hz, 1 H), 6.84 (s, 2 H), 6.78(d, J=8.4 Hz, 1 H), 6.41(d, J=8.8 Hz, 1 H), 3.98-3.93(m, 2 H), 3.904 (s, 3 H), 3.07-3.05(m, 2 H) |
| 413 | 403.0 | 9.27 (s, 1 H), 8.95 (s, 1 H), 7.95 (s, 1 H), 7.54 (s, 1 H), 7.49 - 7.46 (m, 3 H), 7.27 (d, J = 9.2 Hz, 1 H), 7.07 - 7.03 (m, 2 H), 6.95 (bs, 2 H), 3.94 (s, 3 H). |
| 414 | 334.0 | 8.88 (d, J= 4.8 Hz, 1H), 8.30 (d, J= 7.2 Hz, 2H), 7.89 (s, 1H), 7.48 - 7.43 (dd, 2H), 7.32 - 7.29 (m, 1H), 6.94 (t, J= 7.2 Hz, 1H), 6.62 (s, 2H), 4.08 (d, J= 5.6 Hz, 2H), 3.75 (s, 3H). |
| 415 | 345.0 | 9.0 (d, J= 4.4 Hz, 1 H) 8.96 (s, 1 H), 7.6 - 7.55 (m, 4 H), 7.35(d, J= 4.4 Hz, 1 H), 7.28 (s, 1 H), 7.15 (t, J= 6.4 Hz, 1 H), 6.64 (s, 2 H), 4.19 (d, J= 6 Hz, 2 H), 3.98 (s, 3 H). |
| 416 | 358.2 | 8.82 (d, J= 4.8 Hz, 1 H), 7.76 (d, J= 9.2 Hz, 1 H), 7.54 (d, J = 8 Hz, 2 H), 7.46 (d, J = 8.4 Hz, 2 H), 7.27 - 7.15 (m, 4 H), 6.53 (s, 2 H), 4.59 - 4.45 (m, 1 H), 3.91 (s, 3 H), 1.45 (d, J= 7.2 Hz, 3 H). |
| 417 | 360.2 | 9.37 (bs, 1 H), 8.59 (d, J= 9.2 Hz, 1 H), 8.27 (d, J= 5.2 Hz, 1 H), 7.94 (bs, 1 H), 7.25 (d, J= 8.4 Hz, 2 H), 7.09 (d, J= 8.4 Hz, 2 H), 6.98 (bs, 2 H), 6.91 (d, J= 8.8 Hz, 1 H), 6.32 (d, J= 5.6 Hz, 1 H), 4.43 (d, J= 5.6 Hz, 2 H), 3.92 (s, 3 H). |
| 418 | 385.2 | 8.69 (d, J = 5.6 Hz, 1 H), 7.79 (d, J= 9.2Hz,1 H), 7.38 (d, J= 2.4 Hz, 1 H), 7.24 (s, 1 H), 7.18 - 7.19 (m, 2 H), 6.93 -6.90 (m, 1 H), 6.85 (bs, 2 H), 6.32 (d, J=8.4 Hz ,1 H), 4.06 (t, J= 7.6 Hz, 2 H), 3.91 ( s, 3 H), 3.15 (t, J = 8.4 Hz, 2 H), 3.02 (s, 3 H) |
| 419 | 377.9 | 8.87(d, J= 4.4 Hz, 1 H), 7.63 (s, 1 H), 7.47 (d, J= 8.0 Hz, 2 H), 7.38 (d, J= 8.0 Hz, 1 H), 7.28 (d, J= 9.2 Hz, 1 H), 7.24-7.18(s, 3 H), 6.69(s, 2 H), 4.18 (d, J= 6.0 Hz, 2 H), 3.91 (s, 3 H) |
| 420 | 380.1 | 8.56 (s, 1 H), 7.86 (d, J= 8.8 Hz, 1 H), 7.27 (s, 1 H), 7.14 (d, J= 8.4 Hz, 1 H), 6.82 (s, 1 H), 6.73 (s, 2 H), 3.21 - 3.05 (m, 8 H), 1.78 (bs, 6 H) |
| 421 | 377.2 | 8.67 (s, 1 H), 8.56 (d, J = 8.4 Hz, 1 H), 8.11(s, 1 H), 7.44(d, J= 8.4 Hz, 1 H), 7.12(d, J = 8.8 Hz , 1 H), 7.10 - 6.95 (m, 3 H), 6.83 (d, J= 8.0 Hz, 1 H), 6.58 (d, J= 4.4 Hz, 1 H), 4.01 (s, 3 H), 3.78 (s, 3 H) |

### Biological Assays

### Example B1: ENPP1 Enzyme Assay With cGAMP Substrate

Ectonucleotide pyrophosphatase/phosphodiesterase 1 (ENPP-1) is a transmembrane glycoprotein that hydrolyzes nucleotides and nucleotide derivatives with the formation of nucleoside-5’-monophosphates. ENPP-1 hydrolyzes 2’3’-cGAMP (cGAMP), breaking it down into 5’-AMP and 5’-GMP. The 5’-AMP formed from the reaction is detected using the AMP-Glo^{®} Kit (Promega). The assay kit contains two reagents. The first reagent terminates the enzymatic reaction, removes ATP (using adenylyl cyclase), and converts 5’-AMP produced into ADP (using polyphosphate: AMP phosphotransferase). The second reagent converts ADP to ATP (using adenylate kinase) and generates light from ATP using the luciferin/luciferase reaction. The amount of light measured is proportional to the amount of 5’-AMP produced by ENPP1.

Different concentrations of ENPP1 inhibitors are pre-incubated with 5 ng/well of human ENPP-1 enzyme (R&D Systems) for 15 minutes at 37° C. The reaction is initiated by adding 20 µM 2’3’-cGAMP and incubating for 30 minutes at 37° C. The final assay reaction mixture contains a buffer of 50 mM Tris pH 8.0, 250 mM NaCl, 0.5 mM CaCl₂, 1 µM ZnCl₂ and 1% DMSO. At the end of the incubation, the reaction is stopped by adding 12 µ1 of AMP-Glo reagent-1 and mixing the reaction uniformly for 5 minutes, followed by incubation at room temperature for one hour. Then 25 µ1 of AMP Glo reagent-2 is added to the reaction, mixed uniformly with a pipette, and incubated at room temperature for one hour to convert the ADP formed from reagent-1 to ATP and light. The generated light is measured in a Perkin Elmer Victor^{®} instrument. Maximal activity control samples (containing enzyme, substrate, and buffer in the absence of ENPP1 inhibitors: MAX) and background control samples (containing enzyme, substrate, and buffer plus a fully inhibitory concentration (3 µM) of the reference ENPP1 Inhibitor, Example 333: MIN) are simultaneously evaluated in order to calculate the percent inhibition at each compound concentration as follows:

$$\begin{matrix}\% \text{inhibition=} \left(\left(\left[\text{MAX-MIN}\right] - \left[\text{COMPOUND-MIN}\right]\right)\right) / \\ \left(\left[\text{MAX-MIN}\right]\right) * 100\end{matrix}$$

The IC₅₀ values for percent inhibition versus compound concentration are determined by fitting the inhibition curves using a four-parameter variable slope model in GraphPad Prism^{®} software. Ki values are derived from the IC₅₀ values using the Cheng-Prusoff equation:

$$\text{Ki} \text{=} {\text{IC}}_{50} / \left(1 + \left[\text{cGAMP}\right] / \text{Km}\right) ,$$

where routinely [cGAMP] = 20 µM and Km = 16 µM

### Example B2: ENPP1 Enzyme Assay With TMP-pNP Substrate

Ectonucleotide pyrophosphatase/phosphodiesterase 1 (ENPP-1) is a transmembrane glycoprotein that hydrolyzes nucleotides and nucleotide derivatives with the formation of nucleotide-5’-monophosphates. ENPP-1 hydrolyzes thymidine 5’monophosphate p-nitrophenyl ester (TMP-pNP) to nucleotide-5’-monophosphate and p-nitrophenol, which is a chromogenic product. The amount of p-nitrophenol product formed is measured using its absorbance at 405 nm, which is directly proportional to enzyme activity. Different concentrations of inhibitors are pre-incubated with 15 ng/well of human ENPP-1 enzyme (R&D Systems) for 15 minutes at 37° C. The reaction is initiated by adding 200 µM TMP-pNP and incubating for 10 minutes at 37° C. The final assay reaction mixture contains a buffer of 50 mM Tris pH 8.0, 250 mM NaCl, 0.5 mM CaCl₂, 1 µM ZnCl₂ and 1% DMSO. The amount of product formed is measured directly in a Tecan^{®} spectrophotometer. Maximal activity control samples (containing enzyme, substrate, and buffer in the absence of ENPP1 inhibitors: MAX) and background control samples (containing enzyme, substrate, and buffer plus a fully inhibitory concentration (3 µM) of the reference ENPP1 Inhibitor, Example 333: MIN) are simultaneously evaluated in order to calculate the percent inhibition at each compound concentration as follows:

$$\begin{matrix}\% \text{inhibition} = ( ( [ \text{MAX} - \text{MIN} ] - [ \text{COMPOUND} - \text{MIN} ] ) \\ / [ \text{MAX} - \text{MIN])} ∗ 100\end{matrix}$$

The IC₅₀ values for percent inhibition versus compound concentration are determined by fitting the inhibition curves (percent inhibition versus inhibitor concentration) using a four-parameter variable slope model in GraphPad Prism^{®} software. Ki values are derived from the IC₅₀ values using the Cheng-Prusoff equation:

$${\text{Ki=IC}}_{50} / \left(1 + \left[\text{TMP-pNP}\right] / \text{Km}\right) ,$$

where routinely [TMP-pNP] = 200 µM and Km = 151 µM

### Example B3: ENPP1 Enzyme Assay With AMP-pNP Substrate

### Hydrolysis of ATP Analog

ENPP1 is an ectonucleotidase that hydrolyzes both the STING activator 2’,3’-cGAMP and 5’ATP (ATP). In some instances, an inhibitor of ENPP-1 is capable of selectively blocking the hydrolysis of 2’,3’-cGAMP while only minimally inhibiting the hydrolysis of ATP. The ATP analog p-Nitrophenyl 5’-Adenosine Monophosphate (AMP-pNP) has been demonstrated to accurately reflect the response of ATP itself to different classes of ENPP1 inhibitors₁ and was synthesized as described in Lee at al₁. The ENPP1 assay with AMP-pNP substrate is conducted in a buffer containing 50 mM Tris-HCl (pH 8.5)/ 250 mM NaCl/0.5 mM CaCl₂/1 µM ZnCl₂/0.1% DMSO. Inhibitors are added at final concentrations ranging between 10 µM and 30 pM depending on the compound. Duplicate wells are run at each inhibitor concentration. The final assay volume is 40 µL and human recombinant ENPP1 is present at 60 ng/well. The assay is initiated by the addition of substrate (300 µM AMP-pNP final concentration), and incubated for 20 minutes at 37° C. The absorbance at 405 nm is then read in a Tecan® plate reader. Each assay plate also includes wells with no enzyme added (MIN OD) and wells with no inhibitor added (MAX OD). The percent inhibition of ENPP1 for each sample is then calculated as:

$$\begin{matrix}\% \text{inhinition=} \left\{\left[\text{Average} \text{of} \left(\text{MAX} \text{OD-MIN OD}\right) -\right)\right) \\ \left(\left(\text{sample} \text{OD} - \text{Average} \text{MIN} \text{OD}\right)\right] / \text{Average of} \left(\text{MAX} \text{} \text{OD} -\right) \\ \left(\left(\text{MIN} \text{OD}\right)\right\} \times 100 \% .\end{matrix}$$

IC50 values of compounds were calculated by entering the percent inhibition values into a sigmoidal variable slope nonlinear regression model in GraphPad Prism® software. IC50 values were converted to Ki values using the Cheng-Prusoff equation₂, where the Kₘ was 151 µM, based on internal determinations

$${K}_{i} = \frac{{\text{IC}}_{50}}{1 + \frac{[ \text{S} ]}{{K}_{m}}}$$

### Indirect Quantitation of 2’,3’-cGAMP Hydrolysis

Hydrolysis of 2‘,3’-cGAMP by ENPP1 generates the products 5‘-GMP and 5’-AMP. In some instances ENPP1 activity with 2’,3’-cGAMP substrate is measured using the AMP-Glo^{Tm} Assay kit₃ to quantitate the production of 5’-AMP. The AMP-Glo^{Tm} Assay Kit contains two reagents that are added sequentially. The first converts the 5’-AMP produced in the reaction to 5’ADP. The second converts the 5’-ADP to 5’ATP and reacts the 5’-ATP with the luciferase/luciferin pair to produce the luminescence signal. The ENPP1 assay with 2’,3’-cGAMP substrate is conducted in a buffer containing 50 mM Tris-HCl (pH 8.5)/ 250 mM NaCl/0.5 mM CaCl₂/1 µM ZnCl₂/0.1% DMSO. Inhibitors are added at final concentrations ranging between 10 µM and 30 pM depending on the compound. Duplicate wells are run at each inhibitor concentration. The final assay volume is 18 µL and human recombinant ENPP1 is present at 5 ng/well. The assay is initiated by the addition of substrate (20 µM AMP-pNP final concentration), and incubated for 30 minutes at 37° C. To stop the reaction, 12 µl of AMP-Glo reagent I is added and the plate is incubated for 60 minutes at room temperature. 25 µl of AMP-detection reagent is then added and the wells are again incubated for 60 minutes at room temperature. The luminescence signal is then measured using a plate-reading luminometer. Each assay plate also includes wells with no enzyme added (MIN OD) and wells with no inhibitor added (MAX OD). The percent inhibition of ENPP1 for each sample is then calculated as:

$$\begin{matrix}\% \text{inhinition=} \left\{\left[\text{Average} \text{of} \left(\text{MAX} \text{OD-MIN OD}\right) -\right)\right) \\ \left(\left(\text{sample} \text{OD} - \text{Average} \text{MIN} \text{OD}\right)\right] / \text{Average of} \left(\text{MAX} \text{} \text{OD} -\right) \\ \left(\left(\text{MIN} \text{OD}\right)\right\} \times 100 \% .\end{matrix}$$

IC₅₀ values of compounds were calculated by entering the percent inhibition values into a sigmoidal variable slope nonlinear regression model in GraphPad Prism® software. IC₅₀ values were converted to Ki values using the Cheng-Prusoff equation₂ where the Kₘ was 15 µM, based on internal determinations.

$${K}_{i} = \frac{{\text{IC}}_{50}}{1 + \frac{[ \text{S} ]}{{K}_{m}}}$$

Calculation of substrate selectivity rations: The inhibition constants (Ki values) of ENPP1 inhibitors toward cGAMP and ATP hydrolysis were determined in the enzyme assays described above in Examples 1 and 2. The selectivity ratios for inhibition of cGAMP hydrolysis versus ATP hydrolysis were then calculated as follows:

$$\text{Selectivity} \text{ratio} \text{for} \text{cGAMP} \text{=} \text{Ki} \left(\text{ATP}\right) / \text{Ki} \left(\text{cGAMP}\right)$$

The data for select compounds is shown in Table 2:

TABLE 2

| Ex. | cGAMP: % Inhibition at 1 µM | cGAMP: % Inhibition at 10 µM | cGAMP: Ki (nM) | TMP-pNP: % inhibition at 10 uM | TMP-pNP: Ki (nM) |
|---|---|---|---|---|---|
| 1 | | | * | | ** |
| 2 | A | | | A | |
| 3 | C | | | B | |
| 5 | B | | ** | B | |
| 6 | C | | ** | | *** |
| 7 | D | | | | |
| 9 | D | | | | |
| 11 | D | | | | |
| 12 | D | | | | |
| 13 | C | | | | |
| 14 | D | | | | |
| 15 | | NA | | | |
| 16 | | NA | | | |
| 17 | | D | | D | |
| 18 | | NA | | D | |
| 19 | A | | | A | |
| 20 | A | | * | | * |
| 21 | C | | | | |
| 22 | | | * | | *** |
| 23 | | D | | D | |
| 24 | | NA | | D | |
| 25 | | A | *** | | *** |
| 26 | D | | | | |
| 28 | | | * | | ** |
| 29 | | A | * | | * |
| 30 | | A | * | A | ** |
| 31 | A | | ** | | *** |
| 32 | A | | | | |
| 33 | | A | ** | | *** |
| 34 | A | | * | | * |
| 35 | | A | ** | | *** |
| 40 | A | | | | |
| 42 | | | ** | | *** |
| 43 | A | | * | | * |
| 45 | D | | | | |
| 46 | D | | | | |
| 48 | C | | | | |
| 49 | C | | | | |
| 51 | A | | ** | | |

% inhibition: A ≥ 75%; 75% > B ≥ 50%; 50% > C ≥ 25%; and 25% > D.
Ki: * < 100 nm; 100 nm < ** < 1 µm; and 1 µm < ***.
NA = Not active.

The data for select compounds is shown in Table 3:

TABLE 3

| Ex. | ENPP1 Inhibition ₋cGAMP: Ki (nM) | ENPP1 Inhibition _AMP-pNP: Ki (nM) | Substrate Selectivity: Ki_AMP-pNP/Ki_cGAMP |
|---|---|---|---|
| 1 | A | B | 41.7 |
| 2 | A | C | >39.0 |
| 3 | B | | |
| 4 | C | | |
| 5 | B | C | >24 |
| 6 | B | | |
| 7 | C | | |
| 8 | C | | |
| 9 | C | | |
| 10 | C | C | |
| 11 | C | C | |
| 12 | C | | |
| 13 | C | | |
| 14 | C | | |
| 15 | C | | |
| 16 | C | | |
| 17 | C | | |
| 18 | C | | |
| 19 | A | C | >95.8 |
| 20 | A | B | 489 |
| 21 | C | | |
| 22 | A | C | >115 |
| 23 | C | | |
| 24 | C | | |
| 25 | C | | |
| 26 | C | | |
| 27 | C | | |
| 28 | A | B | 31.2 |
| 29 | A | A | 19.2 |
| 30 | A | C | 39.4 |
| 31 | B | | |
| 32 | A | A | 24.3 |
| 33 | B | | |
| 34 | A | B | 17.4 |
| 35 | B | | |
| 36 | C | | |
| 37 | C | | |
| 38 | C | | |
| 39 | C | | |
| 40 | A | B | 24.1 |
| 41 | C | | |
| 42 | B | C | >96.1 |
| 43 | A | B | 22.9 |
| 44 | C | | |
| 45 | C | | |
| 46 | C | | |
| 47 | C | | |
| 48 | C | | |
| 49 | C | | |
| 50 | C | | |
| 51 | B | | |
| 52 | A | B | 58.1 |
| 54 | A | B | 36.9 |
| 55 | A | B | 86.5 |
| 56 | A | A | 6.3 |
| 57 | A | A | 24.7 |
| 58 | A | A | 26.9 |
| 59 | A | A | 29.6 |
| 60 | A | A | 16.7 |
| 61 | A | A | 23.7 |
| 62 | A | A | 16.1 |
| 63 | A | A | 28.0 |
| 64 | A | A | 11.9 |
| 65 | A | A | 38.7 |
| 66 | A | A | 35.1 |
| 67 | A | A | 67.2 |
| 68 | A | B | >37.0 |
| 69 | A | A | 8.9 |
| 70 | A | A | 15.5 |
| 71 | A | A | 13.3 |
| 72 | A | A | 11.9 |
| 73 | A | A | 24.2 |
| 74 | A | A | 22.5 |
| 75 | A | B | 41.3 |
| 76 | A | | |
| 77 | A | | |
| 78 | A | | |
| 79 | A | | |
| 80 | A | | |
| 86 | A | B | >43.2 |
| 113 | B | | |
| 114 | A | C | >42.7 |
| 115 | B | | |
| 116 | C | | |
| 117 | B | | |
| 118 | C | | |
| 119 | C | | |
| 120 | A | C | 54.5 |
| 121 | A | C | >61.5 |
| 122 | A | B | 21.9 |
| 123 | C | | |
| 124 | C | | |
| 125 | A | C | >562 |
| 126 | B | C | >134 |
| 127 | C | | |
| 128 | B | | |
| 129 | C | | |
| 130 | C | | |
| 131 | C | | |
| 132 | B | | |
| 133 | C | | |
| 134 | A | C | >69.1 |
| 135 | B | | |
| 136 | A | C | >461 |
| 137 | A | B | 22.4 |
| 138 | B | | |
| 139 | C | | |
| 140 | A | B | 25.1 |
| 141 | A | C | >929 |
| 142 | C | | |
| 143 | C | | |
| 144 | A | B | 189.2 |
| 145 | A | B | 20.7 |
| 146 | B | | |
| 147 | B | C | >117 |
| 148 | A | A | 10.1 |
| 149 | A | A | 12.2 |
| 150 | B | C | >158 |
| 151 | A | | |
| 152 | C | | |
| 153 | A | C | 62.9 |
| 154 | C | | |
| 155 | B | | |
| 156 | A | B | 23.3 |
| 157 | A | | |
| 158 | A | B | 16.3 |
| 159 | A | A | 54.8 |
| 160 | A | A | 13.9 |
| 161 | C | | |
| 162 | A | | |
| 163 | C | | |
| 164 | A | A | 14.8 |
| 165 | C | | |
| 166 | B | | |
| 167 | A | B | 10.6 |
| 168 | C | | |
| 169 | A | B | 6.9 |
| 170 | C | | |
| 171 | B | C | >50.7 |
| 172 | C | | |
| 173 | C | | |
| 174 | B | C | |
| 175 | A | C | >63.3 |
| 176 | C | | |
| 177 | C | | |
| 178 | C | | |
| 179 | C | | |
| 180 | B | C | > 11.9 |
| 181 | C | | |
| 182 | A | B | 24.7 |
| 183 | C | | |
| 184 | B | C | > 18.0 |
| 185 | B | C | > 8.00 |
| 186 | C | | |
| 187 | C | | |
| 188 | B | | |
| 189 | B | C | > 27.0 |
| 190 | C | C | |
| 191 | B | C | > 25.0 |
| 192 | C | | |
| 193 | A | C | > 38.6 |
| 194 | C | | |
| 195 | B | C | > 45.7 |
| 196 | A | C | > 13.4 |
| 197 | A | C | >49.7 |
| 198 | A | B | 39.2 |
| 199 | A | B | 33.6 |
| 200 | B | C | >64.2 |
| 201 | A | B | > 19.9 |
| 202 | A | B | 10.5 |
| 203 | B | C | > 33.7 |
| 204 | B | C | > 39.4 |
| 205 | B | C | > 14.4 |
| 206 | A | B | 39.6 |
| 207 | A | A | 36.8 |
| 208 | A | C | > 16.4 |
| 209 | C | | |
| 210 | C | | |
| 211 | C | | |
| 212 | A | C | > 46.1 |
| 213 | A | C | 33.0 |
| 214 | B | C | > 15.3 |
| 215 | C | | |
| 216 | A | C | > 72.3 |
| 217 | B | C | > 18.2 |
| 218 | B | C | >42.9 |
| 219 | A | B | 26.4 |
| 220 | A | B | 35.9 |
| 221 | C | | |
| 222 | B | C | > 14.6 |
| 223 | C | | |
| 224 | B | B | > 2.40 |
| 225 | A | A | 48.4 |
| 226 | A | B | 29.5 |
| 227 | A | B | 51.9 |
| 228 | C | | |
| 229 | A | A | 27.2 |
| 230 | A | A | 71.2 |
| 231 | A | A | 30.9 |
| 232 | B | C | > 7.1 |
| 233 | C | | |
| 234 | C | | |
| 235 | B | C | >97.7 |
| 236 | A | B | 109 |
| 237 | A | C | >371 |
| 238 | A | B | >99.7 |
| 239 | A | B | 62 |
| 240 | A | C | >81.3 |
| 241 | A | B | 49.0 |
| 242 | B | C | >82.4 |
| 243 | C | | |
| 244 | A | B | 53.0 |
| 245 | B | C | >92.6 |
| 246 | A | C | >87.7 |
| 247 | B | C | 67.3 |
| 248 | A | A | 48.6 |
| 249 | B | C | |
| 250 | A | C | > 89.3 |
| 251 | C | | |
| 252 | C | | |
| 253 | A | B | 79.4 |
| 254 | A | C | > 31.4 |
| 255 | C | | |
| 256 | A | C | >118 |
| 257 | C | | |
| 258 | A | C | >48.1 |
| 259 | A | A | 41.6 |
| 260 | A | A | 21.5 |
| 261 | B | C | >12.9 |
| 262 | B | C | >173 |
| 263 | B | C | >61.9 |
| 264 | B | C | >28.0 |
| 265 | B | C | >13.0 |
| 266 | A | B | >5.9 |
| 267 | A | B | 22.5 |
| 268 | C | | |
| 269 | A | B | >7.1 |
| 270 | C | | |
| 271 | B | C | >16.2 |
| 272 | A | C | >35.2 |
| 273 | B | C | >12.4 |
| 274 | B | C | >6.4 |
| 275 | B | C | >7.7 |
| 276 | C | | |
| 277 | B | C | >90.4 |
| 278 | C | | |
| 279 | A | B | 24.2 |
| 280 | C | | |
| 281 | A | B | 30.7 |
| 282 | A | A | |
| 283 | A | A | 5.4 |
| 284 | A | A | 28.6 |
| 285 | A | B | >28.3 |
| 286 | C | | |
| 287 | C | | |
| 288 | A | B | 27.9 |
| 289 | A | C | > 626 |
| 290 | A | B | 17.1 |
| 291 | B | C | > 126 |
| 292 | A | C | >31.1 |
| 293 | A | A | 17.9 |
| 294 | B | C | >119 |
| 295 | C | | |
| 296 | A | C | > 40.3 |
| 297 | A | B | 19.5 |
| 298 | C | | |
| 299 | A | C | >45.9 |
| 300 | A | B | 13.7 |
| 301 | A | B | 34.2 |
| 302 | A | B | >13.0 |
| 303 | A | B | 41.6 |
| 304 | A | C | >34.0 |
| 305 | A | B | >13.0 |
| 306 | A | B | 22.4 |
| 307 | B | C | >15.5 |
| 308 | A | B | 28.4 |
| 309 | B | | |
| 310 | A | | |
| 311 | A | C | >133 |
| 312 | A | B | 28.2 |
| 313 | A | C | >38.8 |
| 314 | A | C | 35.7 |
| 315 | A | C | >49.7 |
| 316 | A | C | >533 |
| 317 | B | C | >49.9 |
| 318 | C | | |
| 319 | A | C | >35.9 |
| 320 | A | | |
| 321 | C | | |
| 322 | A | | |
| 323 | C | | |
| 324 | B | | |
| 325 | A | | |
| 326 | A | | |
| 327 | A | | |
| 328 | B | | |
| 329 | B | | |
| 330 | A | B | 35 |
| 331 | A | B | 36.3 |
| 332 | C | | |
| 333 | C | | |
| 334 | A | C | >414 |
| 335 | B | | |
| 336 | C | | |
| 337 | C | | |
| 338 | B | | |
| 339 | A | B | 38.5 |
| 340 | C | | |
| 341 | C | | |
| 342 | B | C | >64.9 |
| 344 | C | | |
| 345 | B | | |
| 346 | B | | |
| 347 | B | | |
| 348 | B | | |
| 349 | C | | |
| 351 | A | C | 55 |
| 352 | A | B | 43 |
| 353 | B | C | >74 |
| 354 | A | B | 54 |
| 355 | A | C | >53 |
| 356 | A | B | 40 |
| 357 | A | B | >18 |
| 358 | A | | |
| 359 | A | B | 54 |
| 361 | A | A | 32 |
| 362 | B | C | >216 |
| 363 | A | B | 49 |
| 364 | A | B | 43 |
| 365 | A | B | 18 |
| 366 | C | | |
| 367 | A | C | >35 |
| 368 | A | B | >7 |
| 369 | C | | |
| 371 | A | C | >32 |
| 373 | B | C | >136 |
| 374 | A | C | >138 |
| 375 | A | A | 25 |
| 376 | B | C | >77 |
| 377 | B | C | >89 |
| 378 | A | B | 42 |
| 379 | A | B | >41 |
| 381 | A | B | >38 |
| 382 | A | C | >142 |
| 383 | C | | |
| 384 | A | B | 27 |
| 385 | A | C | >44 |
| 386 | A | B | 39 |
| 387 | A | C | 36 |
| 388 | A | A | 28 |
| 389 | A | A | 34 |
| 391 | C | | |
| 392 | A | C | >133 |
| 393 | A | B | 51 |
| 394 | A | C | >95 |
| 395 | A | B | >31 |
| 396 | B | C | >20 |
| 397 | A | B | >13 |
| 398 | A | B | 33 |
| 399 | C | | |
| 400 | C | | |
| 401 | C | | |
| 402 | A | B | 46 |
| 403 | B | C | >227 |
| 404 | A | B | >103 |
| 405 | C | | |
| 406 | B | C | >65 |
| 407 | B | C | >14 |
| 408 | B | | |
| 409 | C | | |
| 410 | A | B | >42 |
| 411 | C | | |
| 412 | B | C | >271 |
| 413 | B | C | >153 |
| 414 | B | | |
| 415 | B | | |
| 416 | A | B | 50 |
| 417 | A | A | 30 |
| 418 | A | B | >40 |
| 419 | A | C | >70 |
| 421 | A | | |

ENPP1 Inhibition: A ≤ 100 nm; 100 nm < B ≤ 1 µm; and 1 µm < C.

### Example B4: Cell Based Assays

### Peripheral Blood Monocytic Cells (PBMCs)

Blood collection: A healthy male donor between 21-35 years of age was identified (no ongoing or recent infection; no vaccination in the past month; no history of autoimmunity/cancer/transplantation/inflammation; no immune-modulatory medication including NSAIDs/COX inhibitors/allergy medication on the day of the blood draw). Blood was collected in BD Vacutainer® tubes (sodium-heparin) from the healthy volunteer.

Isolation: Blood was diluted with one volume of 1X PBS (phosphate buffered saline from Gibco Cat# 10010-023); it was carefully underlaid with mononuclear cell separation reagent (Histopaque® from Sigma, Cat #10771), brought to room temperature, and centrifuged for 25 min at 2000 rpm and room temperature with brakes off. The interphase layer was collected into a fresh 50 mL tube. Three volumes of PBS were added, and the cells were centrifuged for 10 min at 1600 rpm and room temperature. The supernatant was decanted, and the pellet was resuspended and washed again with one volume of PBS, and centrifuged for 10 min at 1400 rpm and room temperature. The pellet was resuspended in 10 mL complete RPMI -1640 (Thermo, Cat #11875093) and the cells were counted. 400,000 cells were added to each well (96 well round bottom cell culture coming plate, Cat# CLS3799) in a volume of 100 µl and incubated overnight at 37° C., in a 5% CO₂ incubator). ENPP1 inhibitors were added in a volume of 50 µL/well and incubated at 37° C. in a 5% CO₂ incubator for 30 minutes. Activating agents (VACV-70/LyoVec- CDS Agonist, Invivogen Cat # tlrl-vav70c or 2’3’-cGAMP, Invivogen Cat # tlrl-nacga23-5) were added in a volume of 50 µL/well. Plates were incubated at 37° C., in a 5% CO₂ incubator for 3, 6 or 19 hr. The final volume was 200 µL/well. Each condition was tested in triplicate.

Sample processing (Interferonβ mRNA): RNA Isolation was performed as described below according to manufacturer’s instructions using a Qiagen RNA isolation kit (RNeasy® Mini kit, Qiagen Cat # 74106). After incubation, the plate was centrifuged for 10 min at 1500 rpm. The pellet was suspended in 100 µl RLT Lysis Buffer provided in the kit and stored at -80° C. for RNA extraction. RNA was isolated as per the kit protocol. It was quantified using a Nanodrop® spectrophotometer (Model # ND1000, Thermo Fisher). RNA was converted to cDNA using a Bio-Rad cDNA conversion kit (iScript® cDNA synthesis kit from Bio-Rad, Cat #170-8891). Reaction volumes were as follows:

| | |
|---|---|
| Components | Volume per Reaction |
| 5x iScript reaction mix | 2 µl |
| iScript reverse transcriptase | 0.5µl |
| Nuclease-free water | * µl |
| RNA template (200 ng or 300 ng) | * µl |
| Total volume | 10 µl |

* Volume adjusted according to the concentration of RNA

The reaction mixture was incubated in an Eppendorf thermal cycler (Model # Master Cycler®, Eppendorf) under the following conditions:

5 minutes at 25° C.
30 minutes at 46° C.
5 minutes at 85° C.
Hold at 4° C. (optional)

IFNβ gene transcription was quantitated using SYBR reagent from Bio-Rad Cat # 172-5120). The cDNA synthesized was diluted (based on the yield from Nanodrop data) to achieve a working concentration of 10 ng/µl using Nuclease free water, and 2.5 µl (25 ng total) was taken as a template for amplification via Real Time PCR (QuantStudio 6 Flex System®). Interferonβ mRNA levels were normalized to β-actin mRNA.

Reaction volumes were as follows:

| | |
|---|---|
| Components | Volume per Reaction |
| 2x SYBR reaction mix | 5 µl |
| Primer (60 nM) | 0.6 µl |
| Nuclease-free water | 1.9 µl |
| cDNA template 25 ng | 2.5 µl |
| Total volume | 10 µl |

The mixture was incubated in the QuantStudio 6 Flex System® as follows: 30 sec at 95° C. (Denaturation); 30 sec at 60° C. (Annealing); and 45 sec at 72° C. (Extension), for 40 amplification cycles. Melt curve analysis was performed in the range of 60° C.-95° C., 0.05° C. increment.

Data Analysis and interpretation: Data were analyzed using the relative quantification method.

The amplification plots were inspected and the baseline and threshold values were adjusted to determine the threshold cycles (CT) for the amplification curves.

The Delta Ct (ΔCt) value between target/experiment gene and housekeeping/Reference gene was calculated for each sample.

$$Δ \text{Ct=Ct (Target gene) - Ct (Reference gene)}$$

Calculate FOE

$$\text{FOE} = {2}^{( - Δ \text{Ct)}}$$

Average FOE values between samples (replicates).

Calculate Fold change

$$\begin{matrix}\text{Fold} \text{Change} = \\ \text{Average} \text{FOE} ( \text{Target} \text{gene} ) / \text{Average} \text{FOE} \text{(Reference gene)}\end{matrix}$$

Primer details:

| SEQ ID NO. | Primer Name | Primer Sequence 5′ to 3′ |
|---|---|---|
| 1 | IFN-β Forward | CAACTTGCTTGGATTCCTACAAAG |
| 2 | IFN-β Reverse | TATTCAAGCCTCCCATTCAATTG |
| 3 | h- Actin forward | CATTCCAAATATGAGATGCGTTGT |
| 4 | h- Actin reverse | TGTGGACTTGGGAGAGGACT |

### Human Foreskin Fibroblasts

The cell line was sourced from ATCC (Cat# SCRC-1041). Cells were cultured in 15% fetal bovine serum-containing growth medium. They were seeded in a 48 well plate @ 100,000 per well (300 µL), and incubated for 1 hour at 37° C., 5% CO₂. 100 µl of compound (0.05 µM to 5 µM final concentrations) was added 30 min prior to addition of cGAMP (12.5 µM & 25 µM final concentrations). The plate was incubated for 3 hrs at 37° C., 5% CO₂. Cells were washed with PBS and 250 µl of RLT reagent (provided in the RNA isolation kit (RNeasy Mini kit, Qiagen Cat # 74106). was added to each well. RNA was isolated using this kit as per the given protocol. RNA was converted to cDNA using a Bio-Rad cDNA conversion kit (iScript® cDNA synthesis kit, Bio-Rad Cat #170-8891). IFN βgene transcription was quantitated using SYBR green dye in an RT-PCR instrument. The methods and protocol were the same as for PBMCs, except that Interferon mRNA levels were normalized to GAPDH mRNA.

Primer details:

| SEQ ID NO. | Primer Name | Primer Sequence 5′ to 3′ |
|---|---|---|
| 5 | h-GAPDH forward | GCTCAGAACACCTATGGGGAA |
| 6 | h-GAPDH reverse | CATCGCCCCACTTGATTTTGG |

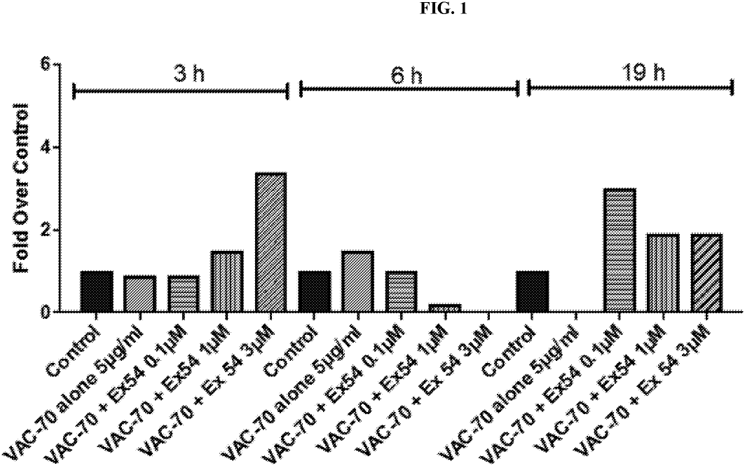

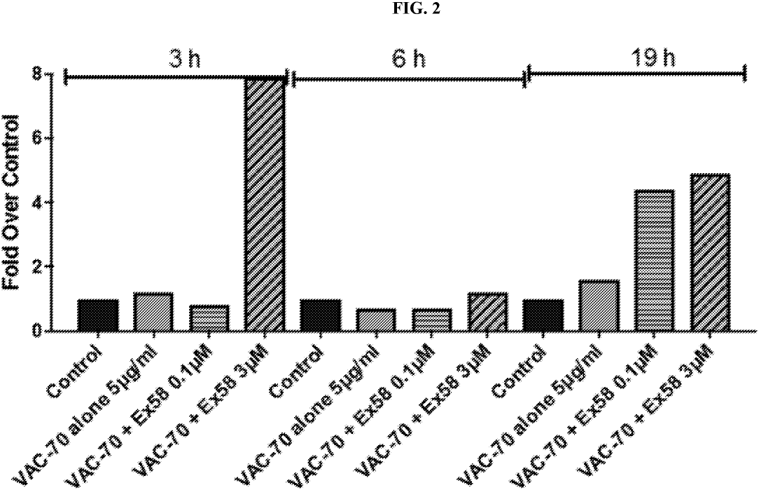

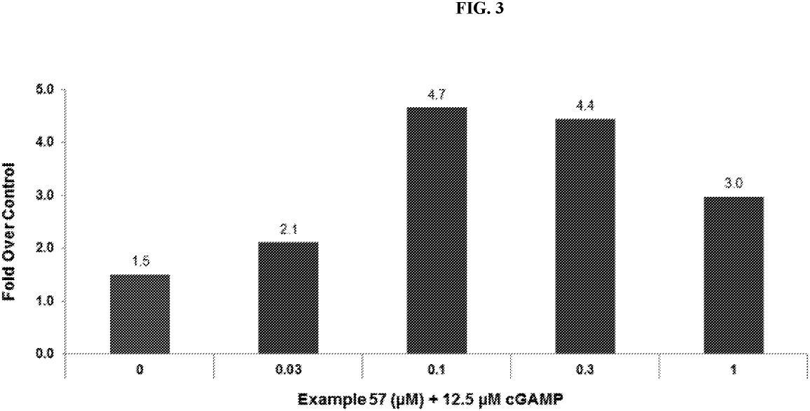

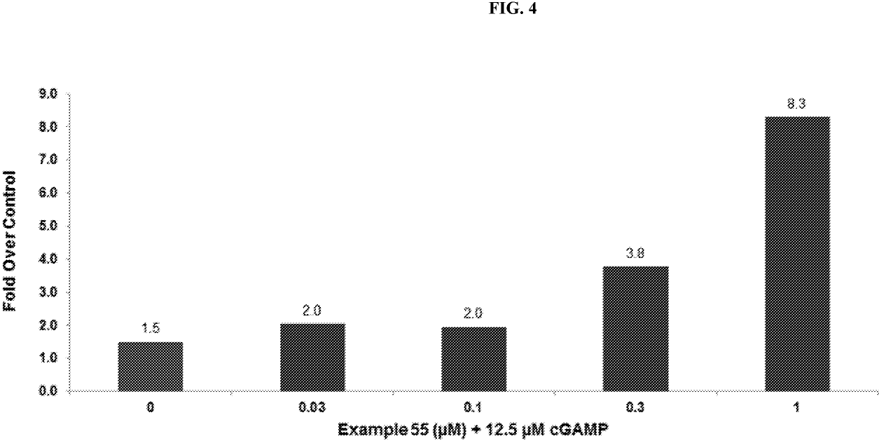

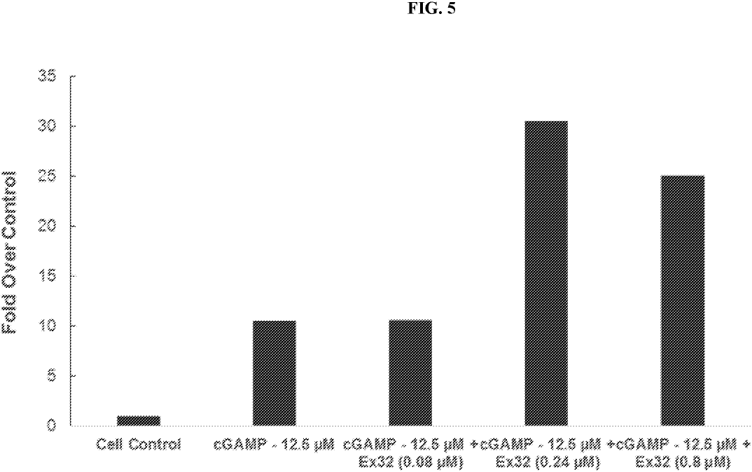

The results of the ENPP1 blockade by examples 32, 54, 55, 57, and 58 are shown in FIG. 1 through FIG. 5.

Abbreviations:
IP-10: C-X-C motif chemokine 10 (CXCL10).
IFN β: Interferon beta.
cGAMP: Guanosine-Adenosine 2′,3′-cyclic monophosphate.
VACV-70: 70 base pair oligonucleotide derived from vaccinia virus.
PBMCs: Peripheral blood mononuclear cells.
HFF-1: Human foreskin fibroblast cell line.

1. A compound of Formula (X), or a pharmaceutically acceptable salt, solvate, or stereoisomer thereof: 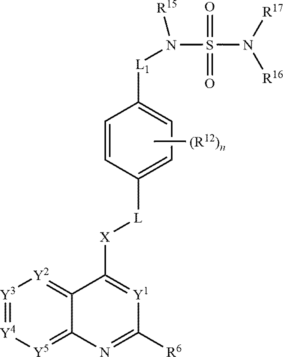
wherein
X is -NR⁷-, -O-, -S-, —S(═O)—, —S(═O)₂—, or -CR⁸R⁹-;
L is a bond or -CR¹⁰R¹¹-;
L₁ is a bond or -CR¹³R¹⁴-;
Y¹ is -N- or -CR¹-;
Y² is -N- or -CR²-;
Y³ is -N- or -CR³-;
Y⁴ is -N- or -CR⁴-;
Y⁵ is -N- or -CR⁵-;
R¹, R², R³, R⁴, R⁵, and R⁶ are independently hydrogen, deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁷ is hydrogen, -CN, -OR^{b}, —C(═O)R^{a}, —C(═O)OR^{b}, —C(═O)NR^{c}R^{d}, —S(═O)R^{a}, —S(═O)₂R^{a}, —S(═O)₂NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
R⁸ and R⁹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, or optionally substituted heterocycloalkyl;
R¹⁰ and R¹¹ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R¹² is independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R⁷ and one R¹² are taken together to form an optionally substituted heterocycloalkyl or an optionally substituted heteroaryl; and the remaining R¹² are independently deuterium, halogen, -CN, -OR^{b}, -NO₂, -NR^{c}R^{d}, —C(═O)R^{a}, —C(═O)OR^{b}, —C(═O)NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl
R¹³ and R¹⁴ are independently hydrogen, deuterium, halogen, -CN, -OR^{b}, -NO₂, -NR^{c}R^{d}, optionally substituted C₁-C₆ alkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
n is 0-4;
R¹⁵ is hydrogen, deuterium, C₁-C₆ alkyl, C₁-C₆ haloalkyl, optionally substituted C₂-C₆ alkenyl, optionally substituted C₂-C₆ alkynyl, optionally substituted cycloalkyl, or optionally substituted heterocycloalkyl;
R¹⁶ and R¹⁷ are independently hydrogen, —C(═O)R^{a}, —C(═O)OR^{b}, —C(═O)NR^{c}R^{d}, C₁-C₆ alkyl, C₁-C₆ haloalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{a} is optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
each R^{b} is hydrogen, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl; and
each R^{c} and R^{d} are each independently hydrogen, deuterium, optionally substituted C₁-C₆ alkyl, optionally substituted C₁-C₆ deuteroalkyl, optionally substituted C₂-C₆ alkenyl, optionally substituted C₂-C₆ alkynyl, optionally substituted cycloalkyl, optionally substituted heterocycloalkyl, optionally substituted aryl, or optionally substituted heteroaryl;
or R^{c} and R^{d} are taken together with the nitrogen atom to which they are attached to form an optionally substituted heterocycloalkyl;
provided that the compound is not: 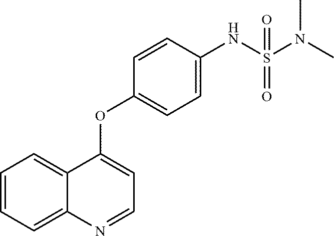 [SMILES: CN(C)S(=O)(=O)Nc1ccc(Oc2ccnc3ccccc23)cc1]
or 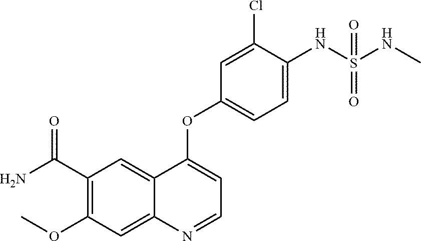 [SMILES: CNS(=O)(=O)Nc1ccc(Oc2ccnc3cc(OC)c(C(N)=O)cc23)cc1Cl]
.

2. The compound of claim 1, or a pharmaceutically acceptable salt, solvate, or stereoisomer thereof, wherein:
X is -NR⁷-.

3. The compound of claim 1, or a pharmaceutically acceptable salt, solvate, or stereoisomer thereof, wherein:
R⁷ is hydrogen.

4. The compound of claim 1, or a pharmaceutically acceptable salt, solvate, or stereoisomer thereof, wherein:
X is -O-.

5. The compound of claim 1, or a pharmaceutically acceptable salt, solvate, or stereoisomer thereof, wherein:
L is a bond.

6. The compound of claim 1, or a pharmaceutically acceptable salt, solvate, or stereoisomer thereof, wherein:
L is -CR⁸R⁹-.

7. The compound of claim 1, or a pharmaceutically acceptable salt, solvate, or stereoisomer thereof, wherein:
R⁸ and R⁹ are independently hydrogen, deuterium, halogen, or optionally substituted C₁-C₆ alkyl.

8. The compound of claim 1, or a pharmaceutically acceptable salt, solvate, or stereoisomer thereof, wherein:
R⁸ and R⁹ are independently hydrogen or C₁-C₆ alkyl.

9. The compound of claim 1, or a pharmaceutically acceptable salt, solvate, or stereoisomer thereof, wherein:
each R¹² is independently deuterium, halogen, -CN, -OR^{b}, -NR^{c}R^{d}, or C₁-C₆ alkyl.

10. The compound of claim 1, or a pharmaceutically acceptable salt, solvate, or stereoisomer thereof, wherein:
each R¹² is independently halogen.

11. The compound of claim 1, or a pharmaceutically acceptable salt, solvate, or stereoisomer thereof, wherein:
n is 0-2.

12. The compound of claim 1, or a pharmaceutically acceptable salt, solvate, or stereoisomer thereof, wherein:
n is 0.

13. The compound of claim 1, or a pharmaceutically acceptable salt, solvate, or stereoisomer thereof, wherein:
R⁷ and one R¹² are taken together to form an optionally substituted heterocycloalkyl.

14. The compound of claim 1, or a pharmaceutically acceptable salt, solvate, or stereoisomer thereof, wherein:
L₁ is a bond.

15. The compound of claim 1, or a pharmaceutically acceptable salt, solvate, or stereoisomer thereof, wherein:
L₁ is -CR¹³R¹⁴-.

16. The compound of claim 1, or a pharmaceutically acceptable salt, solvate, or stereoisomer thereof, wherein:
R¹³ and R¹⁴ are independently hydrogen, deuterium, halogen, or optionally substituted C₁-C₆ alkyl.

17. The compound of claim 1, or a pharmaceutically acceptable salt, solvate, or stereoisomer thereof, wherein:
R¹³ and R¹⁴ are hydrogen.

18. The compound of claim 1, or a pharmaceutically acceptable salt, solvate, or stereoisomer thereof, wherein:
R¹⁵ is hydrogen, C₁-C₆ alkyl, or cycloalkyl.

19. The compound of claim 1, or a pharmaceutically acceptable salt, solvate, or stereoisomer thereof, wherein:
R¹⁶ and R¹⁷ are independently hydrogen, C₁-C₆ alkyl, or cycloalkyl.

20. The compound of claim 1, or a pharmaceutically acceptable salt, solvate, or stereoisomer thereof, wherein:
R¹⁶ and R¹⁷ are hydrogen.

21. The compound of claim 1, or a pharmaceutically acceptable salt, solvate, or stereoisomer thereof, wherein: 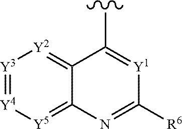
is 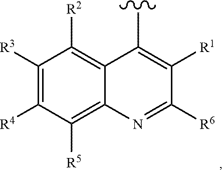 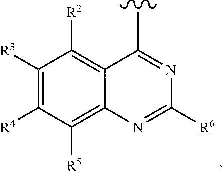 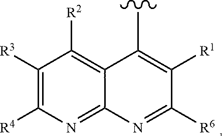 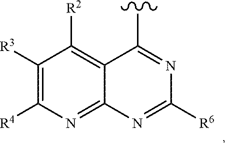 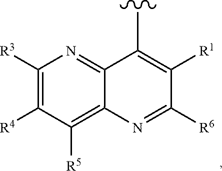
or 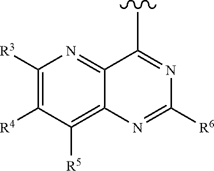
.

22. The compound of claim 1, or a pharmaceutically acceptable salt, solvate, or stereoisomer thereof, wherein: 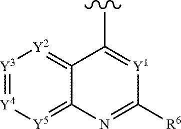
is 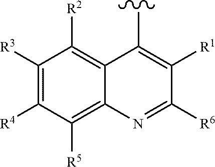
or 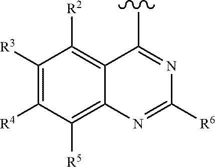
.

23. The compound of claim 1, or a pharmaceutically acceptable salt, solvate, or stereoisomer thereof, wherein:
R¹ is hydrogen, deuterium, halogen, -CN, -OR^{b}, -NR^{c}R^{d}, or C₁-C₆ alkyl.

24. The compound of claim 1, or a pharmaceutically acceptable salt, solvate, or stereoisomer thereof, wherein:
R¹ is hydrogen, halogen, or -CN.

25. The compound of claim 1, or a pharmaceutically acceptable salt, solvate, or stereoisomer thereof, wherein:
R² is hydrogen, deuterium, halogen, -CN, -OR^{b}, -NR^{c}R^{d}, or C₁-C₆ alkyl.

26. The compound of claim 1, or a pharmaceutically acceptable salt, solvate, or stereoisomer thereof, wherein:
R² is hydrogen.

27. The compound of claim 1, or a pharmaceutically acceptable salt, solvate, or stereoisomer thereof, wherein:
R³ is hydrogen, deuterium, halogen, -CN, -OR^{b}, -NR^{c}R^{d}, or C₁-C₆ alkyl.

28. The compound of claim 1, or a pharmaceutically acceptable salt, solvate, or stereoisomer thereof, wherein:
R³ is hydrogen, -OR^{b}, or halogen.

29. The compound of claim 1, or a pharmaceutically acceptable salt, solvate, or stereoisomer thereof, wherein:
R³ is hydrogen or -OR^{b}.

30. The compound of claim 1, or a pharmaceutically acceptable salt, solvate, or stereoisomer thereof, wherein:
R⁴ is hydrogen, deuterium, halogen, -CN, -OR^{b}, -NR^{c}R^{d}, or C₁-C₆ alkyl.

31. The compound of claim 1, or a pharmaceutically acceptable salt, solvate, or stereoisomer thereof, wherein:
R⁴ is hydrogen or -OR^{b}.

32. The compound of claim 1, or a pharmaceutically acceptable salt, solvate, or stereoisomer thereof, wherein:
R⁴ is -OR^{b}.

33. The compound of claim 1, or a pharmaceutically acceptable salt, solvate, or stereoisomer thereof, wherein:
R⁵ is hydrogen, deuterium, halogen, -CN, -OR^{b}, -NR^{c}R^{d}, or C₁-C₆ alkyl.

34. The compound of claim 1, or a pharmaceutically acceptable salt, solvate, or stereoisomer thereof, wherein:
R⁵ is hydrogen.

35. The compound of claim 1, or a pharmaceutically acceptable salt, solvate, or stereoisomer thereof, wherein:
R⁶ is hydrogen, deuterium, halogen, -CN, -OR^{b}, -NR^{c}R^{d}, or C₁-C₆ alkyl.

36. The compound of claim 1, or a pharmaceutically acceptable salt, solvate, or stereoisomer thereof, wherein:
R⁶ is hydrogen.

37-144. (canceled)
